(19) United States
(12) Patent Application Publication
(10) Pub. No.: US 2017/0037086 A1
(43) Pub. Date: Feb. 9, 2017

(54) PEPTIDOMIMETIC MACROCYCLES WITH PTH ACTIVITY

(71) Applicant: AILERON THERAPEUTICS, INC., Cambridge, MA (US)

(72) Inventors: Noriyuki Kawahata, West Roxbury, MA (US); Manoj Samant, Burlington, MA (US); Eric Feyfant, Lexington, MA (US)

(21) Appl. No.: 15/302,984

(22) PCT Filed: Apr. 9, 2015
(86) PCT No.: PCT/US2015/025089

§ 371 Date: Oct. 8, 2016

Related U.S. Application Data
(60) Provisional application No. 61/977,387, filed on Apr. 9, 2014.
(60) Provisional application No. 61/977,391, filed on Apr. 9, 2014.
(60) Provisional application No. 62/048,928, filed on Sep. 11, 2014.

(51) Int. Cl. C07K 14/00; A61K 38/12; A61K 9/00

(52) U.S. Cl. CPC C07K 14/001; A61K 9/0019; A61K 38/12; A61K 38/00

(57) ABSTRACT

The present invention provides peptidomimetic macrocycles capable of modulating parathyroid hormone levels and methods of using such macrocycles for the treatment of disease.

### CROSS-REFERENCE

This application claims priority to U.S. Provisional Application No. 61/977,387, filed Apr. 9, 2014; U.S. Provisional Application No. 61/977,391, filed Apr. 9, 2014; and U.S. Provisional Application No. 62/048,928, filed Sep. 11, 2014, which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Apr. 2, 2015, is named 35224-791.601_SL.txt and is 557,559 bytes in size.

### BACKGROUND OF THE INVENTION

Oversecretion of parathyroid hormone (PTH) is the key disease driver in primary (PHPT) and secondary (SHPT) hyperparathyroidism. Parathyroid glands are part of the endocrine system and produce PTH. PTH regulates the levels of calcium, phosphorus, and magnesium, in the bloodstream, maintaining an appropriate balance of these substances, which is essential for normal bone mineralization.

PTH is a peptide secreted from the parathyroid glands. Its amino acid sequence and the nucleotide sequence of the related gene are known. PTH acts through the PTH/parathyroid-related protein (PTHrP) receptor to promote bone resorption and decrease calcium excretion. Human parathyroid hormone (hPTH) circulates as substantially intact hPTH1-84. Full length hPTH1-84 and fragment hPTH1-34 are believed to be biologically active, while fragment hPTH35-84 is believed to be inactive. Fragments lacking the N-terminus of PTH (hPTH7-84 or hPTH7-34) are not only inactive, but can also inhibit biologically active PTH in vivo.

### SUMMARY OF THE INVENTION

The present invention provides pharmaceutical formulations comprising an effective amount of peptidomimetic macrocycles or pharmaceutically acceptable salts thereof. The peptidomimetic macrocycles provided herein are cross-linked (e.g., stapled) and possess improved pharmaceutical properties relative to their corresponding uncross-linked peptidomimetic macrocycles. These improved properties include improved bioavailability, enhanced chemical and in vivo stability, increased potency, and reduced immunogenicity (i.e. fewer or less severe injection site reactions).

In one aspect, a composition is provided comprising a peptidomimetic macrocycle comprising at least one macrocycle-forming linker and an amino acid sequence with at least about 60%, 65%, 70%, 75%, 80%, 85%, 90%, 95%, 96%, 97%, 98%, 99% or 100% sequence identity to a sequence of Table 1a, 1b, 2a, or 2b, wherein the peptidomimetic macrocycle comprises at least two non-natural amino acids connected by a first macrocycle-forming linker of the at least one macrocycle-forming linker.

In some embodiments, the first macrocycle-forming linker connects amino acids 7 and 11, 7 and 14, 8 and 12, 9 and 13, 10 and 14, 11 and 15, 12 and 16, 13 and 17, 14 and 18, 14 and 21, 15 and 19, 15 and 22, 17 and 24, 18 and 22, 18 and 25, 22 and 26, 22 and 29, 24 and 28, 25 and 32, 26 and 30, 26 and 33, or 27 and 31. In some embodiments, the first macrocycle-forming linker connects amino acids 7 and 11, 8 and 12, 9 and 13, 10 and 14, 13 and 17, 14 and 18, or 18 and 22. In some embodiments, the first macrocycle-forming linker connects amino acids 9 and 13. In some embodiments, the first macrocycle-forming linker connects amino acids 10 and 14 or 11 and 15.

In one aspect, a composition is provided comprising a peptidomimetic macrocycle comprising at least one macrocycle-forming linker and an amino acid sequence with at least about 60%, 65%, 70%, 75%, 80%, 85%, 90%, 95%, 96%, 97%, 98%, 99% or 100% sequence identity to a sequence of Table 1a, wherein the peptidomimetic macrocycle comprises at least two non-natural amino acids connected by a first macrocycle-forming linker of the at least one macrocycle-forming linker, wherein the first macrocycle-forming linker connects amino acids 10 and 14 or 11 and 15.

In some embodiments, the first macrocycle-forming linker connects amino acids 13 and 17. In some embodiments, the first macrocycle-forming linker connects amino acids 14 and 18. In some embodiments, the first macrocycle-forming linker connects amino acids 18 and 22. In some embodiments, the first macrocycle-forming linker connects amino acids 24 and 28 or 27 and 31.

In one aspect, a composition is provided comprising a peptidomimetic macrocycle comprising at least one macrocycle-forming linker and an amino acid sequence with at least about 60%, 65%, 70%, 75%, 80%, 85%, 90%, 95%, 96%, 97%, 98%, 99% or 100% sequence identity to a sequence of Table 1a, wherein the peptidomimetic macrocycle comprises at least two non-natural amino acids connected by a first macrocycle-forming linker of the at least one macrocycle-forming linker, wherein the first macrocycle-forming linker connects amino acids 24 and 28 or 27 and 31.

In some embodiments, the at least one macrocycle-forming linker comprises a second macrocycle-forming linker. In some embodiments, the second macrocycle-forming linker connects amino acids 18 and 22, 22 and 26, 24 and 28, or 26 and 30.

In some embodiments, the second macrocycle-forming linker connects amino acids 22 and 26. In some embodiments, the second macrocycle-forming linker connects amino acids 24 and 28. In some embodiments, the second macrocycle-forming linker connects amino acids 26 and 30.

In some embodiments, the second macrocycle-forming linker connects amino acids 18 and 22 or 24 and 28. In some embodiments, a first macrocycle-forming linker connects amino acids 8 and 12, and the second macrocycle-forming linker connects amino acids 22 and 26. In some embodiments, the first macrocycle-forming linker connects amino acids 13 and 17, and the second macrocycle-forming linker connects amino acids 22 and 26. In some embodiments, the first macrocycle-forming linker connects amino acids 13 and 17, and the second macrocycle-forming linker connects amino acids 24 and 28. In some embodiments, the first macrocycle-forming linker connects amino acids 14 and 18, and the second macrocycle-forming linker connects amino acids 22 and 26. In some embodiments, a first macrocycle-forming linker connects amino acids 7 and 11, and the second macrocycle-forming linker connects amino acids 22 and 26.

In some embodiments, the at least one macrocycle-forming linker comprises a third macrocycle-forming linker. In some embodiments, the third macrocycle-forming linker connects amino acids 27-31.

In one aspect, a composition is provided comprising a peptidomimetic macrocycle having an amino acid sequence with at least about 60%, 65%, 70%, 75%, 80%, 85%, 90%, 95%, 96%, 97%, 98%, 99% or 100% sequence identity to a sequence of Table 7. In some embodiments, the peptidomimetic macrocycle has an amino acid sequence with at least about 75% sequence identity to a sequence of Table 7. In some embodiments, the peptidomimetic macrocycle has an amino acid sequence with at least about 90% sequence identity to a sequence of Table 7. In some embodiments, the peptidomimetic macrocycle has an amino acid sequence with at least about 95% sequence identity to a sequence of Table 7. In some embodiments, the peptidomimetic macrocycle has 100% sequence identity to a sequence of Table 7.

In one aspect, a composition is provided comprising a peptidomimetic macrocycle having an amino acid sequence with at least about 60%, 65%, 70%, 75%, 80%, 85%, 90%, 95%, 96%, 97%, 98%, 99% or 100% sequence identity to a sequence of Table 3b. In some embodiments, the peptidomimetic macrocycle has an amino acid sequence with at least about 75% sequence identity to a sequence of Table 3b. In some embodiments, the peptidomimetic macrocycle has an amino acid sequence with at least about 90% sequence identity to a sequence of Table 3b. In some embodiments, the peptidomimetic macrocycle has an amino acid sequence with at least about 95% sequence identity to a sequence of Table 3b. In some embodiments, the peptidomimetic macrocycle has an amino acid sequence with 100% sequence identity to a sequence of Table 3b.

In one aspect, a composition is provided comprising a peptidomimetic macrocycle having an amino acid sequence with at least about 60%, 65%, 70%, 75%, 80%, 85%, 90%, 95%, 96%, 97%, 98%, 99% or 100% sequence identity to a sequence of Table 6. In some embodiments, the peptidomimetic macrocycle has an amino acid sequence with at least about 75% sequence identity to a sequence of Table 6. In some embodiments, the peptidomimetic macrocycle has an amino acid sequence with at least about 90% sequence identity to a sequence of Table 6. In some embodiments, the peptidomimetic macrocycle has an amino acid sequence with at least about 95% sequence identity to a sequence of Table 6. In some embodiments, the peptidomimetic macrocycle has an amino acid sequence with 100% sequence identity to a sequence of Table 6.

In one aspect, a composition is provided comprising a peptidomimetic macrocycle having an amino acid sequence with at least about 60%, 65%, 70%, 75%, 80%, 85%, 90%, 95%, 96%, 97%, 98%, 99% or 100% sequence identity to a sequence of Table 8. In some embodiments, the peptidomimetic macrocycle has an amino acid sequence with at least about 75% sequence identity to a sequence of Table 8. In some embodiments, the peptidomimetic macrocycle has an amino acid sequence with at least about 90% sequence identity to a sequence of Table 8. In some embodiments, the peptidomimetic macrocycle has an amino acid sequence with at least about 95% sequence identity to a sequence of Table 8. In some embodiments, the peptidomimetic macrocycle has an amino acid sequence with 100% sequence identity to a sequence of Table 8. In some embodiments, the peptidomimetic macrocycle is a peptidomimetic macrocycle of Table 8.

In one aspect, a composition is provided comprising a peptidomimetic macrocycle of Formula (I):

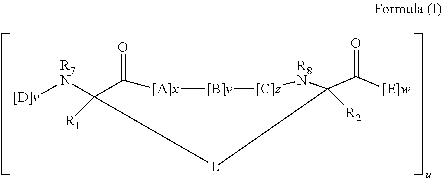

wherein: each A, C, D, and E is independently an amino acid; each B is independently an amino

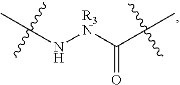

acid, [—NH-L₃-CO—], [—NH-L₃-SO₂—], or [—NH-L₃-]; each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids; each R₃ is independently —H, alkyl, alkenyl, alkynyl, arylalkyl, heteroalkyl, cycloalkyl, heterocycloalkyl, cycloalkylalkyl, aryl, or heteroaryl, optionally substituted with R₅; each L and L′ is independently a macrocycle-forming linker of the formula -L₁-L₂-,

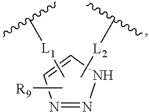

or -L₁-S-L₂-S-L₃-; each L₁, L₂ and L₃ is independently alkylene, alkenylene, alkynylene, heteroalkylene, cycloalkylene, heterocycloalkylene, arylene, heteroarylene, or [—R₄—K—R₄—]ₙ, each being optionally substituted with R₅; when L is not

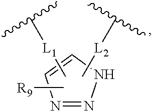

or -L₁-S-L₂-S-L₃-, L₁ and L₂ are alkylene, alkenylene, alkynylene, heteroalkylene, cycloalkylene, heterocycloalkylene, arylene, or heteroarylene; each R₄ is independently alkylene, alkenylene, alkynylene, heteroalkylene, cycloalkylene, heterocycloalkylene, arylene, or heteroarylene; each K is independently O, S, SO, SO₂, CO, CO₂ or CONR₃; each R₅ is independently halogen, alkyl, —OR₆, —N(R₆)₂, —SR₆, —SOR₆, —SO₂R₆, —CO₂R₆, a fluorescent moiety, a radioisotope or a therapeutic agent; each R₆ is independently —H, alkyl, alkenyl, alkynyl, arylalkyl, cycloalkylalkyl, heterocycloalkyl, a fluorescent moiety, a radioisotope or a therapeutic agent; each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue; each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue; each R₉ is independently alkyl, alkenyl, alkynyl, aryl, cycloalkyl, cycloalkenyl, heteroaryl, or heterocyclyl group, unsubstituted or optionally substituted with Rₐ and/or R_{b}; Rₐ and R_{b} are independently alkyl, OCH₃, CF₃, NH₂, CH₂NH₂, F, Br, I,

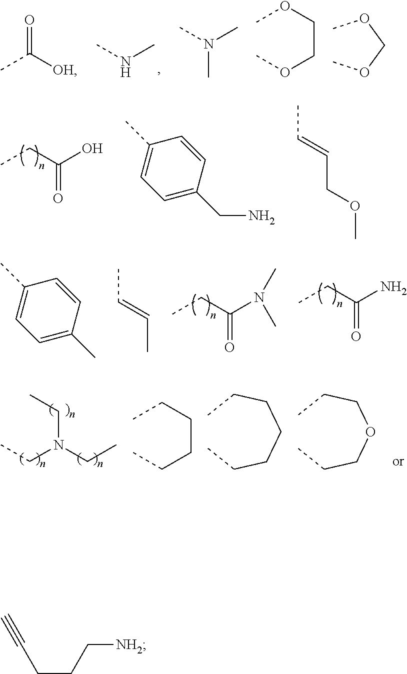

each v and w is independently an integer from 0-1000, for example 0-500, 0-200, 0-100, 0-50, 0-30, 0-20, or 0-10; u is an integer from 1-10, for example 1-5, 1-3 or 1-2; each x, y and z is independently an integer from 0-10, for example the sum of x+y+z is 2, 3, 6 or 10; each n is independently an integer from 1-5; and wherein A, B, C, D, and E, taken together with the crosslinked amino acids connected by the macrocycle-forming linker -L₁-L₂-, form an amino acid sequence of the peptidomimetic macrocycle with at least about 60%, 65%, 70%, 75%, 80%, 85%, 90%, 95%, 96%, 97%, 98%, 99% or 100% sequence identity to a sequence of Table 1a, 1b, 2a, or 2b.

In some embodiments, an amino acid sequence of the peptidomimetic macrocycle has at least about 60%, 65%, 70%, 75%, 80%, 85%, 90%, 95%, 96%, 97%, 98%, 99% or 100% sequence identity to a sequence of Table 1a or 3a. In some embodiments, an amino acid sequence of the peptidomimetic macrocycle has at least about 60%, 65%, 70%, 75%, 80%, 85%, 90%, 95%, 96%, 97%, 98%, 99% or 100% sequence identity to a sequence of Table 6 or Table 7.

In some embodiments, u is 1. In some embodiments, the sum of x+y+z is 2, 3 or 6. In some embodiments, the sum of x+y+z is 3 or 6. In some embodiments, each of v and w is independently an integer from 0-200. In some embodiments, each of v and w is independently an integer from 0-10, 0-15, 0-20, 0-25, or 0-30. In some embodiments, L₁ and L₂ are independently alkylene, alkenylene or alkynylene. In some embodiments, L₁ and L₂ are independently C₃-C₁₀ alkylene or alkenylene. In some embodiments, L₁ and L₂ are independently C₃-C₆ alkylene or alkenylene. In some embodiments, L is

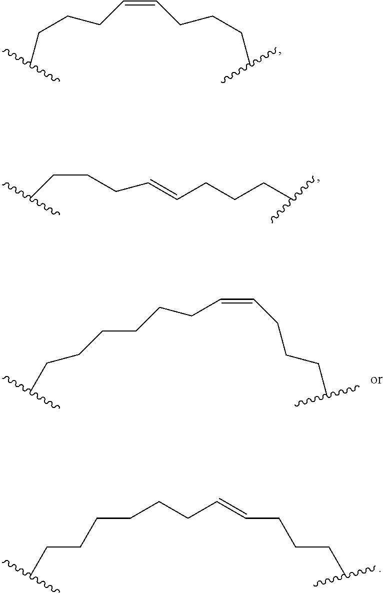 [SMILES: *CCC/C=C\CCC* ; *CCC/C=C/CCC* ; *CCC/C=C\CCCCCC* ; *CCC/C=C/CCCCCC*]

In some embodiments, L is

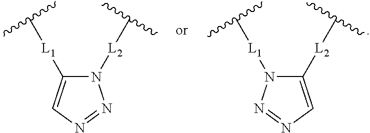

In some embodiments, L is

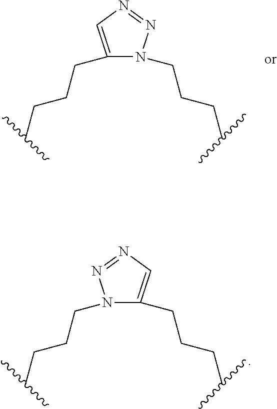 [SMILES: *CCCc1cnnn1CCC* ; *CCCc1cnnn1CCC*]

In some embodiments, R₁ and R₂ are H. In some embodiments, R₁ and R₂ are independently alkyl. In some embodiments, R₁ and R₂ are methyl. In some embodiments, the peptidomimetic macrocycle has Formula (Ia):

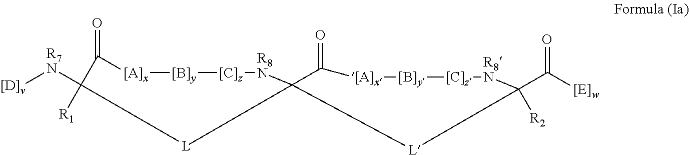

wherein: R₈′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a E residue; and x′, y′ and z′ are independently integers from 0-10.

In some embodiments, u is 2.

In some embodiments, the peptidomimetic macrocycle has the Formula (Ib):

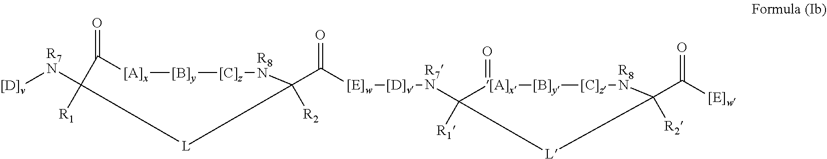

wherein: R₇′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue; R₈′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue; v′ and w′ are independently integers from 0-100; and x′, y′ and z′ are independently integers from 0-10, for example x′+y′+z′ is 2, 3, 6 or 10.

In some embodiments, the sum of x+y+z is 2, 3 or 6, for example 3 or 6. In some embodiments, the sum of x′+y′+z′ is 2, 3 or 6, for example 3 or 6. In some embodiments, each of v and w is independently an integer from 1-10, 1-15, 1-20, or 1-25.

In some embodiments, u is 3.

In some embodiments, the peptidomimetic macrocycle has the Formula (Ic):

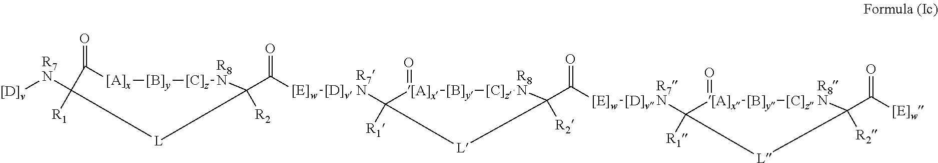

wherein: R₇″ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue; R₈″ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue; v″ and w″ are independently integers from 0-100; and x″, y″ and z″ are independently integers from 0-10, for example x″+y″+z″ is 2, 3, 6 or 10.

In some embodiments, the peptidomimetic macrocycle has the Formula (IIIa) or Formula (IIIb):

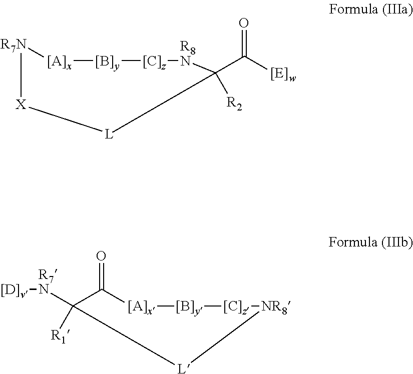

wherein: each A, C, D and E is independently an amino acid; each B is independently an amino acid,

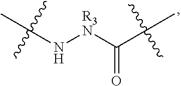

[—NH-L₃-CO—], [—NH-L₃-SO₂—], or [—NH-L₃-]; each R₁′ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or R₂ forms a macrocycle-forming linker L′ connected to the alpha position of one of said E amino acids; each R₃ is independently —H, alkyl, alkenyl, alkynyl, arylalkyl, heteroalkyl, cycloalkyl, heterocycloalkyl, cycloalkylalkyl, aryl, or heteroaryl, optionally substituted with R₅; L and L′ are independently a macrocycle-forming linker of the formula -L₁-L₂-,

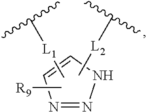

or -L₁-S-L₂-S-L₃-; L₁, L₂ and L₃ are independently alkylene, alkenylene, alkynylene, heteroalkylene, cycloalkylene, heterocycloalkylene, arylene, heteroarylene, or [—R₄—K—R₄—]ₙ, each being optionally substituted with R₅; each R₄ is independently alkylene, alkenylene, alkynylene, heteroalkylene, cycloalkylene, heterocycloalkylene, arylene, or heteroarylene; each K is independently O, S, SO, SO₂, CO, CO₂ or CONR₃; each R₅ is independently halogen, alkyl, —OR₆, —N(R₆)₂, —SR₆, —SOR₆, —SO₂R₆, —CO₂R₆, a fluorescent moiety, a radioisotope or a therapeutic agent; each R₆ is independently —H, alkyl, alkenyl, alkynyl, arylalkyl, cycloalkylalkyl, heterocycloalkyl, a fluorescent moiety, a radioisotope or a therapeutic agent; R₇ or R₇′ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue; R₈ or R₈′ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue; each R₉ is independently alkyl, alkenyl, alkynyl, aryl, cycloalkyl, cycloalkenyl, heteroaryl, or heterocyclyl group, unsubstituted or optionally substituted with Rₐ and/or R_{b}; each Rₐ and R_{b} is independently alkyl, OCH₃, CF₃, NH₂, CH₂NH₂, F, Br, I,

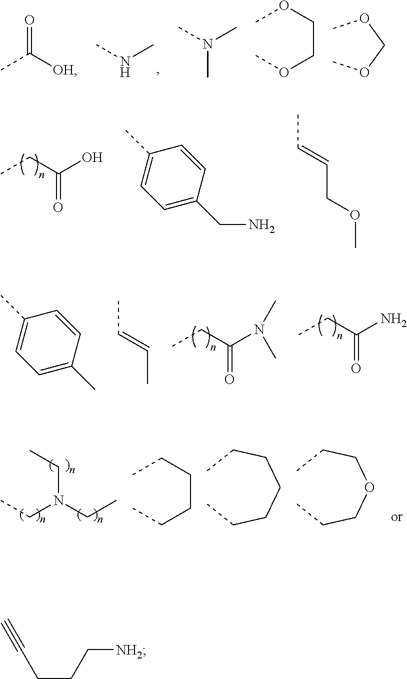

v and w′ are independently integers from 0-1000, for example 0-500, 0-200, 0-100, 0-50, 0-30, 0-20, or 0-10; x, y, z, x′, y′ and z′ are independently integers from 0-10, for example the sum of x+y+z is 2, 3, 6 or 9, or the sum of x′+y′+z′ is 2, 3, 6, or 9; n is an integer from 1-5; X is C═O, CHR_{c}, or C═S; R_{c} is alkyl, alkenyl, alkynyl, arylalkyl, cycloalkyl, cycloalkylalkyl, heteroalkyl, or heterocycloalkyl; and A, B, C, and E, taken together with the crosslinked amino acids connected by the macrocycle-forming linker -L₁-L₂-, form an amino acid sequence of the peptidomimetic macrocycle with at least about 60%, 65%, 70%, 75%, 80%, 85%, 90%, 95%, 96%, 97%, 98%, 99%, or 100% sequence identity to a sequence of Table 1a, 1b, 2a, or 2b.

In some embodiments, the amino acid sequence of the peptidomimetic macrocycle has at least about 60%, 65%, 70%, 75%, 80%, 85%, 90%, 95%, 96%, 97%, 98%, 99%, or 100% sequence identity to a sequence of Table 1a or 3a.

In some embodiments, the peptidomimetic macrocycle has the Formula:

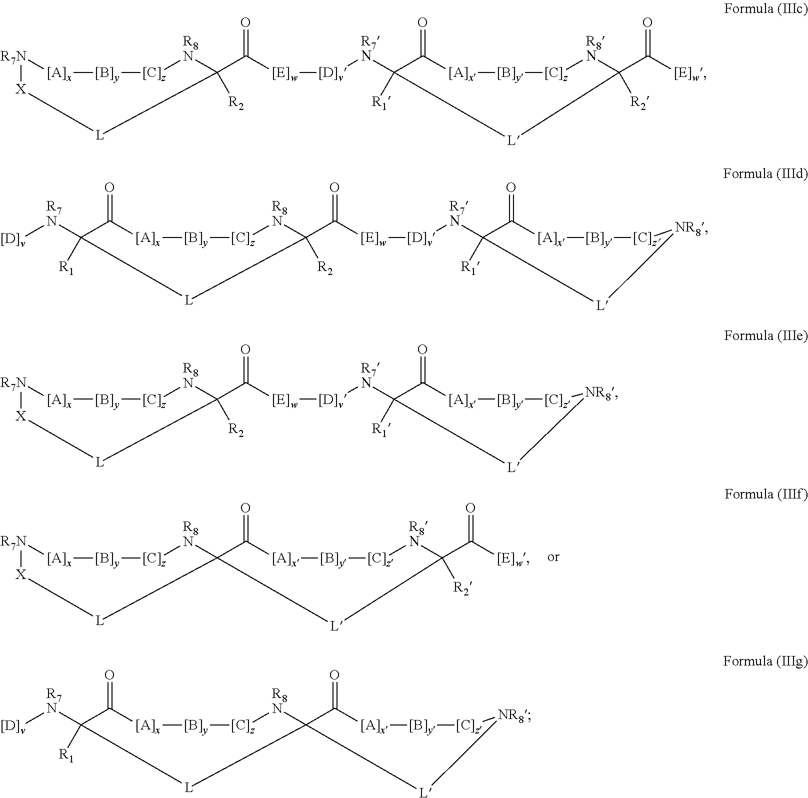

wherein R₁′ and R₂′ are independently —H, alkyl, alkenyl, alkynyl, arylalkyl, cycloalkyl, cycloalkylalkyl, heteroalkyl, or heterocycloalkyl, unsubstituted or substituted with halo-; and v, w, v′ and w′ are independently integers from 0-100.

In some embodiments, L₁ and L₂ are independently alkylene, alkenylene or alkynylene.

In one aspect, a composition is provided comprising a peptidomimetic macrocycle comprising an amino acid sequence of formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH, or a C-terminal capping group; X₁-X₃₆ are absent or are amino acids, such that at least X₇-X₂₂ are not absent; at least three, four, five, six, or seven amino acids from the group consisting of X₂₀, X₂₃, X₂₄, X₂₅, X₂₇, X₂₈, X₃₁, X₃₂, and X₃₄ are selected as follows: X₂₀ is Arg, X₂₃ is Trp or Phe, X₂₄ is Leu, X₂₅ is Arg, X₂₇ is Lys or Leu, X₂₈ is Leu or Ile, X₃₁ is Val or Ile, X₃₂ is His, and X₃₄ is Phe; and wherein the peptidomimetic macrocycle comprises at least one pair of crosslinked amino acids selected from the group consisting of amino acids X₁-X₃₆.

In one aspect, a composition is provided comprising a peptidomimetic macrocycle comprising an amino acid sequence of Formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH, or a C-terminal capping group; X₁-X₃₆ are absent or are amino acids, such that at least X₇-X₂₂ are not absent; at least three, four, five, six, or seven amino acids from the group consisting of X₂₀, X₂₃, X₂₄, X₂₅, X₂₇, X₂₈, X₃₁, X₃₂, and X₃₄ are selected as follows: X₂₀ is Arg or Cit or an analog thereof, X₂₃ is Trp or Phe or Ala or 1Nal or 2Nal, X₂₄ is Leu or Cpg or Cba or Ala or an analog thereof or a crosslinked amino acid, X₂₅ is Arg or His or Aib or Phe or Ser or Glu or Ala or Tyr or Trp or an analog thereof or a crosslinked amino acid, X₂₇ is Lys or Leu or Cit or Nle or hF or Tyr or His or Phe or Gln or an analog thereof or a crosslinked amino acid, X₂₈ is Leu or Ile or Cpg or Cba or Cha or an analog thereof or a crosslinked amino acid, X₃₁ is Val or Ile or Cpg or Cba or Nle or Thr or an analog thereof or a crosslinked amino acid, X₃₂ is His or Tyr or Phe or Ala or 2Pal or an analog thereof or a crosslinked amino acid, and X₃₄ is Phe or Tyr or Ala; and wherein the peptidomimetic macrocycle comprises at least one pair of crosslinked amino acids selected from the group consisting of amino acids X₁-X₃₆.

In one aspect, a composition is provided comprising a peptidomimetic macrocycle comprising an amino acid sequence of Formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH or a C-terminal capping group; X₁-X₃₆ are absent or are amino acids, such that at least X₇-X₂₂ are not absent; A is the amino acid sequence X₀-X₁-X₂-X₃-X₄-X₅-X₆-X₇-X₈-X₉-X₁₀-X₁₁-X₁₂-X₁₃-X₁₄ and comprises at least three amino acids selected from PTH (7-14); B is the amino acid sequence X₁₅-X₁₆-X₁₇-X₁₈-X₁₉-X₂₀-X₂₁ and comprises at least three amino acids selected from PTHrP (15-21); and C is the amino acid sequence X₂₂-X₂₃-X₂₄-X₂₅-X₂₆-X₂₇-X₂₈-X₂₉-X₃₀-X₃₁₋X₃₂-X₃₃-X₃₄-X₃₅-X₃₆-X₃₇ and comprises at least six amino acids selected from PTH (22-34); and wherein the peptidomimetic macrocycle comprises at least one pair of crosslinked amino acids selected from the group consisting of amino acids X₁-X₃₆.

In one aspect, a composition is provided comprising a peptidomimetic macrocycle comprising an amino acid sequence of Formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH or a C-terminal capping group; X₁-X₃₆ are absent or are amino acids, such that at least X₇-X₂₂ are not absent; A is the amino acid sequence X₀-X₁-X₂-X₃-X₄-X₅-X₆-X₇-X₈-X₉-X₁₀-X₁₁-X₁₂-X₁₃-X₁₄ and comprises at least two amino acids selected from PTHrP (7-14); B is the amino acid sequence X₁₅-X₁₆-X₁₇-X₁₈-X₁₉-X₂₀-X₂₁ and comprises at least three amino acids selected from PTHrP (15-21); and C is the amino acid sequence X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇ and comprises at least three amino acids selected from PTH (22-34); and wherein the peptidomimetic macrocycle comprises at least one pair of crosslinked amino acids selected from the group consisting of amino acids X₁-X₃₆.

In one aspect, a composition is provided comprising a peptidomimetic macrocycle comprising an amino acid sequence of Formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH or a C-terminal capping group; X₁-X₃₆ are absent or are amino acids, such that at least X₇-X₂₂ are not absent; A is the amino acid sequence X₀-X₁-X₂-X₃-X₄-X₅-X₆-X₇-X₈-X₉-X₁₀-X₁₁-X₁₂-X₁₃-X₁₄ and comprises at least three amino acids selected from PTHrP (7-14) or at least three amino acids selected from PTHrP (7-14); wherein X₁₀ is not Asn or Asp; X₁₁ is not Asn or Asp, X₁₂ is not Gly, or any combination thereof; B is the amino acid sequence X₁₅-X₁₆-X₁₇-X₁₈-X₁₉-X₂₀-X₂₁ and comprises at least three amino acids selected from PTHrP (15-21); and C is the amino acid sequence X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇ and comprises at least three amino acids selected from PTHrP (22-36) or at least three amino acids selected from PTH (22-34); and wherein the peptidomimetic macrocycle comprises at least one pair of crosslinked amino acids selected from the group consisting of amino acids X₁-X₃₆.

In one aspect, a composition is provided comprising a peptidomimetic macrocycle comprising an amino acid sequence of Formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH or a C-terminal capping group; X₁-X₃₆ are absent or are amino acids, such that at least X₇-X₂₂ are not absent; A is the amino acid sequence X₀-X₁-X₂-X₃-X₄-X₅-X₆-X₇-X₈-X₉-X₁₀-X₁₁-X₁₂-X₁₃-X₁₄ and comprises at least two contiguous amino acids selected from PTHrP (7-14); B is the amino acid sequence X₁₅-X₁₆-X₁₇-X₁₈-X₁₉-X₂₀-X₂₁ and comprises at least three contiguous amino acids selected from PTHrP (15-21); and C is the amino acid sequence X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇ and comprises at least two contiguous amino acids selected from PTHrP (22-36) or at least two contiguous amino acids selected from PTH (22-34); and wherein the peptidomimetic macrocycle comprises at least one pair of crosslinked amino acids selected from the group consisting of amino acids X₁-X₃₆.

In some embodiments, X₀ is —H or an N-terminal capping group, for example acetyl, 1NaAc, 2NaAc, PhAc, a fatty acid, a urea, a sulfonamide, or a polyalkylene oxide linked to the N-terminus of residue X₁. In some embodiments, X₁ is Ser, Ala, Deg, Har, a dialkylated amino acid, Aib, Ac5c, Ac3c, Ac6c, desamino-Ser, desamino-Ac5c, desamino-Aib, Val, an analog thereof, or absent. In some embodiments, X₂ is an aromatic amino acid, Val, Trp, Arg, D-Trp, D-Arg, F4COOH, Bip, F4NH₂, 1Nal, 2Nal, 2Pal, 3Pal, 4Pal, Bpa, Deg, Ile, an analog thereof, or absent. In some embodiments, X₃ is Ser, Deg, Aib, Ac3c, Ac5c, Ac6c, Glu, Lys, Phe, Aib, Gly, Ala, an analog thereof, or absent. In some embodiments, X₄ is Glu, Gln, Phe, His, an analog thereof, or absent. In some embodiments, X₅ is Ile, His, Lys, Glu, Phe, an analog thereof, or absent. In some embodiments, X₆ is Gln, Lys, Glu, Phe, Ala, an analog thereof, or absent. In some embodiments, X₇ is an aromatic amino acid, a hydrophobic amino acid, Leu, Lys, Glu, Ala, Phe, Met, F4Cl, 1Nal, 2Nal, 2Pal, 3Pal, 4Pal, Phe, Nle, an analog thereof, or a crosslinked amino acid. In some embodiments, X₈ is a hydrophobic amino acid, Met, Leu, Nle, an analog thereof, or a crosslinked amino acid. In some embodiments, X₉ is an aromatic amino acid, His, Aib, or an analog thereof. In some embodiments, X₁₀ is Asn, Asp, Gln, Ala, Ser, Val, His, Trp, Aib, an analog thereof, or a crosslinked amino acid. In some embodiments, X₁₁ is a hydrophobic amino acid, a positively charged amino acid, an aromatic amino acid, Leu, Lys, Har, Arg, Ala, Val, Ile, Met, Phe, Trp, D-Trp, Nle, Cit, hK, hL, an analog thereof, or a crosslinked amino acid. In some embodiments, X₁₂ is a D-amino acid, a hydrophobic amino acid, a hydrophilic amino acid, an aromatic amino acid, a positively charged amino acid, a negatively charged amino acid, an uncharged amino acid, Gly, D-Trp, Ala, Aib, Arg, His, Trp, an analog thereof, or a crosslinked amino acid. In some embodiments, X₁₃ is a positively charged amino acid, Lys, Ser, Ala, Aib, Leu, Glu, Gln, Arg, His, Phe, Trp, Pro, Cit, Kfam, Ktam, an analog thereof, or a crosslinked amino acid. In some embodiments, X₁₄ is an aromatic amino acid, His, Ser, Trp, Ala, Leu, Lys, Arg, Phe, Trp, Aib, an analog thereof, or a crosslinked amino acid. In some embodiments, X₁₅ is a hydrophobic amino acid, Leu, Ile, Tyr, Aib, an analog thereof, or a crosslinked amino acid. In some embodiments, X₁₆ is Asn, Gln, Lys, Ala, Glu, an analog thereof, or a crosslinked amino acid. In some embodiments, X₁₇ is Ser, Asp, β-Ala, β-hPhe, Aib, an analog thereof, or a crosslinked amino acid. In some embodiments, X₁₈ is a hydrophobic amino acid, Met, Nle, Leu, β-hIle, hSer(OMe), β-hPhe, Aib, an analog thereof, or a crosslinked amino acid. In some embodiments, X₁₉ is a positively charged amino acid, Glu, Arg, Ser, Aib, Cit, Glu, Ala, an analog thereof, or a crosslinked amino acid. In some embodiments, X₂₀ is a positively charged amino acid, Cit, Arg, Ala, an analog thereof, or a crosslinked amino acid. In some embodiments, X₂₁ is a positively charged amino acid, Cit, Val, Arg, Lys, Gln, Cit, Ala, an analog thereof, or a crosslinked amino acid. In some embodiments, X₂₂ is an aromatic amino acid, Glu, Phe, Ser, Aib, an analog thereof, or a crosslinked amino acid. In some embodiments, X₂₃ is an aromatic amino acid, a hydrophobic amino acid, Trp, Phe, Ala, 9-Aal, 1Nal, 2Nal, an analog thereof, absent, or a crosslinked amino acid. In some embodiments, X₂₄ is an aromatic amino acid, a hydrophobic amino acid, Leu, Ala, Cba, Cpg, Aib, an analog thereof, absent, or a crosslinked amino acid. In some embodiments, X₂₅ is a positively charged amino acid, Cit, Arg, His, Leu, Trp, Tyr, Phe, Ala, Ser, Glu, Aib, an analog thereof, absent, or a crosslinked amino acid. In some embodiments, X₂₆ is a positively charged amino acid, Lys, His, Ala, Phe, Ser, Glu, AmO, AmK, Cit, and Aib an analog thereof, absent, or a crosslinked amino acid. In some embodiments, X₂₇ is a positively charged amino acid, Cit, Lys, Leu, Arg, Nle, Tyr, His, Phe, hF, Leu, Gln, an analog thereof, absent, or a crosslinked amino acid. In some embodiments, X₂₈ is an aromatic amino acid, a hydrophobic amino acid, Leu, Ile, Cba, Cha, Cpg, Aib, an analog thereof, absent, or a crosslinked amino acid. In some embodiments, X₂₉ is Gln, Ala, Glu, Ser, Aib, an analog thereof, absent, or a crosslinked amino acid. In some embodiments, X₃₀ is Asp, Glu, Leu, Arg, hPhe, Asn, His, Ser, Ala, Phe, an analog thereof, absent, or a crosslinked amino acid. In some embodiments, X₃₁ is an aromatic amino acid, a hydrophobic amino acid, Val, Ile, Nle, Thr, Ser, Cba, Cpg, an analog thereof, absent, or a crosslinked amino acid. In some embodiments, X₃₂ is an aromatic amino acid, His, Trp, Arg, Phe, Tyr, Ile, Ala, 2Pal, 3Pal, 4Pal, an analog thereof, absent, or a crosslinked amino acid. In some embodiments, X₃₃ is Asn, Thr, Glu, Asp, Lys, Phe, an analog thereof, absent, or a crosslinked amino acid. In some embodiments, X₃₄ is an aromatic amino acid, a hydrophobic amino acid, Phe, Ala, Tyr, Arg, 2Nal, hF, Glu, Lys, Ser, an analog thereof, absent, or a crosslinked amino acid. In some embodiments, X₃₅ is Glu, Gly, an analog thereof, absent, or a crosslinked amino acid. In some embodiments, X₃₆ is an aromatic amino acid, Tyr, Pra, an analog thereof, absent, or a crosslinked amino acid. In some embodiments, X₃₇ is —OH, or a C-terminal capping group, for example a primary, secondary, or tertiary amino group, an alkyloxy or an aryloxy group.

In some embodiments, X₀ is —H or an N-terminal capping group, for example acetyl, 1NaAc, 2NaAc, PhAc, a fatty acid, a urea, a sulfonamide, or a polyalkylene oxide linked to the N-terminus of residue X₁. In some embodiments, X₁ is Ser, Ala, Deg, Har, a dialkylated amino acid, Aib, Ac5c, Ac3c, Ac6c, desamino-Ser, desamino-Ac5c, desamino-Aib, Val, an analog thereof, or absent. In some embodiments, X₂ is an aromatic amino acid, Val, Trp, Arg, D-Trp, D-Arg, F4COOH, Bip, F4NH₂, 1Nal, 2Nal, 2Pal, 3Pal, 4Pal, Bpa, Deg, Ile, an analog thereof, or absent. In some embodiments, X₃ is Ser, Deg, Aib, Ac3c, Ac5c, Ac6c, Glu, Lys, Phe, Aib, Gly, Ala, an analog thereof, or absent. In some embodiments, X₄ is Glu, Gln, Phe, His, an analog thereof, or absent. In some embodiments, X₅ is Ile, His, Lys, Glu, Phe, an analog thereof, or absent. In some embodiments, X₆ is Gln, Lys, Glu, Phe, Ala, an analog thereof, or absent. In some embodiments, X₇ is an aromatic amino acid, a hydrophobic amino acid, Leu, Lys, Glu, Ala, Phe, F4Cl, 1Nal, 2Nal, 2Pal, 3Pal, 4Pal, Phe, or an analog thereof. In some embodiments, X₈ is a hydrophobic amino acid, Met, Leu, Nle, or an analog thereof. In some embodiments, X₉ is an aromatic amino acid, His, or an analog thereof. In some embodiments, X₁₀ is Asn, Asp, Gln, Ala, Ser, Val, His, Trp, an analog thereof, or a crosslinked amino acid. In some embodiments, X₁₁ is a hydrophobic amino acid, a positively charged amino acid, an aromatic amino acid, Leu, Lys, Har, Arg, Ala, Val, Ile, Met, Phe, Trp, D-Trp or an analog thereof. In some embodiments, X₁₂ is a D-amino acid, a hydrophobic amino acid, a hydrophilic amino acid, an aromatic amino acid, a positively charged amino acid, a negatively charged amino acid, an uncharged amino acid, Gly, D-Trp, Ala, Aib, Arg, His, Trp or an analog thereof. In some embodiments, X₁₃ is a positively charged amino acid, Lys, Ser, Ala, Aib, Leu, Glu, Gln, Arg, His, Phe, Trp, Pro or an analog thereof. In some embodiments, X₁₄ is an aromatic amino acid, His, Ser, Trp, Ala, Leu, Lys, Arg, Phe, Trp, an analog thereof, or a crosslinked amino acid. In some embodiments, X₁₅ is a hydrophobic amino acid, Leu, Ile, Tyr, an analog thereof, or a crosslinked amino acid. In some embodiments, X₁₆ is Asn, Gln, Lys, an analog thereof, or a crosslinked amino acid. In some embodiments, X₁₇ is Ser, Asp, β-Ala, β-hPhe, an analog thereof, or a crosslinked amino acid. In some embodiments, X₁₈ is a hydrophobic amino acid, Met, Nle, Leu, β-hIle, hSer(OMe), β-hPhe, an analog thereof, or a crosslinked amino acid. In some embodiments, X₁₉ is a positively charged amino acid, Cit, Glu, Arg, Ser, an analog thereof, or a crosslinked amino acid. In some embodiments, X₂₀ is a positively charged amino acid, Cit, Arg, an analog thereof, or a crosslinked amino acid. In some embodiments, X₂₁ is a positively charged amino acid, Cit, Val, Arg, Lys, Gln, an analog thereof, or a crosslinked amino acid. In some embodiments, X₂₂ is an aromatic amino acid, Glu, Phe, an analog thereof, or a crosslinked amino acid. In some embodiments, X₂₃ is an aromatic amino acid, a hydrophobic amino acid, Trp, Phe, 9-Aal, 1Nal, 2Nal, an analog thereof, absent, or a crosslinked amino acid. In some embodiments, X₂₄ is an aromatic amino acid, a hydrophobic amino acid, Leu, an analog thereof, absent, or a crosslinked amino acid. In some embodiments, X₂₅ is a positively charged amino acid, Cit, Arg, His, Leu, Trp, Tyr, Phe, an analog thereof, absent, or a crosslinked amino acid. In some embodiments, X₂₆ is a positively charged amino acid, Lys, His, an analog thereof, absent, or a crosslinked amino acid. In some embodiments, X₂₇ is a positively charged amino acid, Cit, Lys, Leu, Arg, Nle, Tyr, His, Phe, hF, Leu, Gln, an analog thereof, absent, or a crosslinked amino acid. In some embodiments, X₂₈ is an aromatic amino acid, a hydrophobic amino acid, Leu, Ile, an analog thereof, absent, or a crosslinked amino acid. In some embodiments, X₂₉ is Gln, Ala, Glu, an analog thereof, absent, or a crosslinked amino acid. In some embodiments, X₃₀ is Asp, Glu, Leu, Arg, hPhe, Asn, His, Ser, an analog thereof, absent, or a crosslinked amino acid. In some embodiments, X₃₁ is an aromatic amino acid, a hydrophobic amino acid, Val, Ile, Nle, Thr, Ser, an analog thereof, absent, or a crosslinked amino acid. In some embodiments, X₃₂ is an aromatic amino acid, His, Trp, Arg, Phe, Tyr, Ile, 2Pal, 3Pal, 4Pal, an analog thereof, absent, or a crosslinked amino acid. In some embodiments, X₃₃ is Asn, Thr, Glu, Asp, Lys, an analog thereof, absent, or a crosslinked amino acid. In some embodiments, X₃₄ is an aromatic amino acid, a hydrophobic amino acid, Phe, Ala, Tyr, Arg, 2Nal, hF, Glu, Lys, an analog thereof, absent, or a crosslinked amino acid. In some embodiments, X₃₅ is Glu, an analog thereof, absent, or a crosslinked amino acid. In some embodiments, X₃₆ is an aromatic amino acid, Tyr, an analog thereof, absent, or a crosslinked amino acid. In some embodiments, X₃₇ is —OH, or a C-terminal capping group, for example a primary, secondary, or tertiary amino group, an alkyloxy or an aryloxy group.

In some embodiments, the peptidomimetic macrocycle comprises at least one macrocycle-forming linker, wherein a macrocycle-forming linker of the at least one macrocycle-forming linker connects the at least one pair of crosslinked amino acids. In some embodiments, the at least one pair of crosslinked amino acids is selected from the group consisting of amino acids X₇-X₃₄. In some embodiments, the at least one macrocycle-forming linker connects amino acids X₉ and X₁₃. In some embodiments, the at least one macrocycle-forming linker connects amino acids X₁₀ and X₁₄. In some embodiments, the at least one macrocycle-forming linker connects amino acids X₁₁ and X₁₅. The peptidomimetic macrocycle of claim wherein the at least one macrocycle-forming linker connects amino acids X₁₂ and X₁₆. The peptidomimetic macrocycle of claim wherein the at least one macrocycle-forming linker connects amino acids X₁₃ and X₁₇. In some embodiments, the at least one macrocycle-forming linker connects amino acids X₁₄ and X₁₈. In some embodiments, the at least one macrocycle-forming linker connects amino acids X₁₈ and X₂₂. In some embodiments, the at least one macrocycle-forming linker connects amino acids X₂₂ and X₂₆. In some embodiments, the at least one macrocycle-forming linker connects amino acids X₂₄ and X₂₈ In some embodiments, the at least one macrocycle-forming linker connects amino acids X₂₆ and X₃₀. In some embodiments, the at least one macrocycle-forming linker connects amino acids X₂₇ and X₃₁.

In some embodiments, the at least one macrocycle-forming linker comprises a first macrocycle-forming linker that connects a first pair of the at least one pair of crosslinked amino acids, and a second macrocycle-forming linker that connects a second pair of the at least one pair of crosslinked amino acids. In some embodiments, X₁₄ and X₁₈ are crosslinked amino acids, and X₂₆ and X₃₀ are crosslinked amino acids. In some embodiments, X₁₄ and X₁₈ are crosslinked amino acids, and X₂₂ and X₂₆ are crosslinked amino acids. In some embodiments, X₁₄ and X₁₈ are crosslinked amino acids, and X₂₄ and X₂₈ are crosslinked amino acids. In some embodiments, X₁₄ and X₁₈ are crosslinked amino acids, and X₂₇ and X₃₁ are crosslinked amino acids. In some embodiments, X₁₃ and X₁₇ are crosslinked amino acids, and X₂₆ and X₃₀ are crosslinked amino acids.

In some embodiments, X₁-X₆ are absent. In some embodiments, X₃₅-X₃₆ are absent.

In some embodiments, each of X₇, X₈, and X₉ is independently a crosslinked amino acid or any amino acid that is a same amino acid at a corresponding position of PTHrP. In some embodiments, each of X₇, X₉, X₁₃, X₂₀, X₂₄, and X₃₂ is independently a crosslinked amino acid or any amino acid that is a same amino acid at a corresponding position of PTH and PTHrP. In some embodiments, X₁₀ is crosslinked or any amino acid except Asn or Asp. In some embodiments, X₁₀ is Gln, Aib, Ala, or Glu. In some embodiments, each of X₁₀, X₁₁, X₁₂, X₁₃, and X₁₄ is independently a crosslinked amino acid or any amino acid that is not a same amino acid at a corresponding position of PTH or PTHrP. In some embodiments, X₁₁ is crosslinked or any amino acid except Leu or Lys. In some embodiments, X₁₁ is Leu. In some embodiments, X₁₁ is Arg or hArg. In some embodiments, X₁₁ is Har. In some embodiments, X₁₂ is crosslinked or any amino acid except Gly. In some embodiments, X₁₂ is Ala or Aib. In some embodiments, X₁₃ is crosslinked or any amino acid except Gly. In some embodiments, X₁₃ is Lys or crosslinked. In some embodiments, X₁₄ is crosslinked or any amino acid except His or Ser. In some embodiments, X₁₄ is a hydrophobic amino acid. In some embodiments, the hydrophobic amino acid is a large hydrophobic amino acid. In some embodiments, X₁₄ is Trp or Phe. In some embodiments, X₁₄ is Phe. In some embodiments, X₁₄ is Tyr. In some embodiments, X₁₄ is crosslinked. In some embodiments, each of X₁₅-X₃₆ is independently a crosslinked amino acid or any amino acid that is a same amino acid at a corresponding position of PTHrP. In some embodiments, each of X₁₃-X₃₆ is independently a crosslinked amino acid or any amino acid that is a same amino acid at a corresponding position of PTHrP. In some embodiments, each of X₁₅, X₁₆, X₁₇, X₁₈, and X₁₉ is independently a crosslinked amino acid or any amino acid that is a same amino acid at a corresponding position of PTHrP. In some embodiments, X₁₈ is a crosslinked amino acid. In some embodiments, X₁₉ is a positively charged amino acid, Cit, Arg. or an analog thereof. In some embodiments, X₁₉ is Arg. In some embodiments, X₂₀ is a positively charged amino acid, Cit, Arg, or an analog thereof. In some embodiments, X₂₀ is Arg. In some embodiments, X₂₁ is a positively charged amino acid, Cit, Arg, Lys, or an analog thereof. In some embodiments, X₂₁ is Arg. In some embodiments, at least two of X₁₉, X₂₀, and X₂₁ comprise a same amino acid at a corresponding position of from PTHrP. In some embodiments, X₁₉-X₂₀-X₂₁ is Arg-Arg-Arg. In some embodiments, an amino acid of the at least one pair of crosslinked amino acids is X₂₂. In some embodiments, X₂₃ is Trp. In some embodiments, X₂₃ is Phe. In some embodiments, X₂₄ is Leu. In some embodiments, X₂₅ is Arg. In some embodiments, X₂₆ is any amino acid except Lys or His. In some embodiments, X₂₆ is Aib. In some embodiments, X₂₆ is Glu. In some embodiments, X₂₇ is Lys. In some embodiments, X₂₇ is Leu. In some embodiments, X₂₈ is Leu. In some embodiments, X₂₈ is Ile. In some embodiments, X₂₉ is Aib. In some embodiments, X₃₁ is Val. In some embodiments, X₃₁ is Ile. In some embodiments, X₃₂ is His. In some embodiments, X₃₃ is Glu. In some embodiments, X₃₃ is Asn. In some embodiments, X₃₃ is Aib or Cit. In some embodiments, X₃₄ is Phe. In some embodiments, X₂₀ is Arg, X₂₃ is Trp, X₂₄ is Leu, X₂₅ is Arg, X₂₇ is Lys, X₂₈ is Leu, X₃₁ is Val, and X₃₄ is Phe. In some embodiments, X₂₀ is Arg, X₂₃ is Phe, X₂₄ is Leu, X₂₇ is Leu, X₂₈ is Ile, X₃₁ is Ile, and X₃₂ is His.

In some embodiments, the macrocycle comprises a contiguous amino acid sequence comprising at least 3 contiguous amino acids that are crosslinked or same amino acids as those at corresponding positions of PTH. In some embodiments, the macrocycle comprises a contiguous amino acid sequence comprising at least 3 contiguous amino acids that are crosslinked or same amino acids as those at corresponding positions of PTHrP. In some embodiments, the macrocycle comprises a contiguous amino acid sequence comprising at most 13 amino acids that are crosslinked or same amino acids as those at corresponding positions of PTH. In some embodiments, the macrocycle comprises a substitution within the contiguous amino acid sequence comprising at most 13 amino acids that are crosslinked or same amino acids as those at corresponding positions of PTH. In some embodiments, the substitution is at X₂₆. In some embodiments, the substitution is at X₂₉. In some embodiments, the substitution is at X₃₃. In some embodiments, the macrocycle comprises at most 10 amino acids that are crosslinked or substitutions, wherein the substitutions are not same amino acids as those at corresponding positions of PTHrP or PTH. In some embodiments, the macrocycle comprises 2 or 4 crosslinked amino acids and at least 3 amino acids that are not same amino acids as those at corresponding positions of PTHrP or PTH. In some embodiments, the macrocycle comprises 3, 4, 5, 6, 7, 8, 9 or 10 amino acids that are crosslinked or substitutions, wherein the substitutions are not same amino acids as those at corresponding positions of PTHrP or PTH.

In one aspect, a composition is provided comprising a peptidomimetic macrocycle selected from Table 3. In one aspect, a composition is provided comprising a peptidomimetic macrocycle selected from Table 7. In one aspect, a composition is provided comprising a peptidomimetic macrocycle selected from Table 6. In one aspect, a composition is provided comprising a peptidomimetic macrocycle selected from Table 8.

In some embodiments, the peptidomimetic macrocycle comprises a helix. In some embodiments, the peptidomimetic macrocycle comprises an α-helix. In some embodiments, the peptidomimetic macrocycle comprises an α,α-disubstituted amino acid. In some embodiments, each amino acid connected by the macrocycle-forming linker is an α,α-disubstituted amino acid.

In some embodiments, the at least one macrocycle-forming linker is a straight chain alkenyl. In some embodiments, the at least one macrocycle-forming linker is a straight chain alkenyl with 6 to 14 carbon atoms. In some embodiments, the at least one macrocycle-forming linker is a straight chain alkenyl with 8 to 12 carbon atoms, for example 8, 9, 10, 11 or 12 carbon atoms. In some embodiments, the at least one macrocycle-forming linker is a C₈ alkenyl with a double bond between C₄ and C₅ of the C₈ alkenyl. In some embodiments, the at least one macrocycle-forming linker is a C₁₂ alkenyl with a double bond between C₄ and C₅ or C₅ and C₆ of the C₁₂ alkenyl.

In some embodiments, the at least one macrocycle-forming linker comprises a first and a second macrocycle-forming linker, wherein the first macrocycle-forming linker connects a first and a second amino acid, wherein the second macrocycle-forming linker connects a third and a fourth amino acid, wherein the first amino acid is upstream of the second amino acid, the second amino acid is upstream of the third amino acid, and the third amino acid is upstream of the fourth amino acid. In some embodiments, 1, 2, 3, 4, 5, 6, or 7, amino acids are between the second and third amino acids. In some embodiments, 4 or 5 amino acids are between the second and third amino acids.

In some embodiments, the at least one macrocycle-forming linker comprises a first and a second macrocycle-forming linker that are separated by 2, 3, 4, 5, 6, or 7 amino acids. In some embodiments, the at least one macrocycle-forming linker comprises a first and a second macrocycle-forming linker that are separated by 4 or 5 amino acids.

In some embodiments, the peptidomimetic macrocycle contains 16-36 amino acids, for example 16, 17, 18, 19, 20, 21, 22, 23, 24, 25, 26, 27, 28, 29, 30, 31, 32, 33, 34, 35 or 36 amino acids. In some embodiments, the peptidomimetic macrocycle contains 24-36 amino acids, for example 24, 25, 26, 27, 28, 29, 30, 31, 32, 33, 34, 35 or 36 amino acids.

In one aspect, a composition is provided comprising a peptidomimetic macrocycle, wherein the peptidomimetic macrocycle is

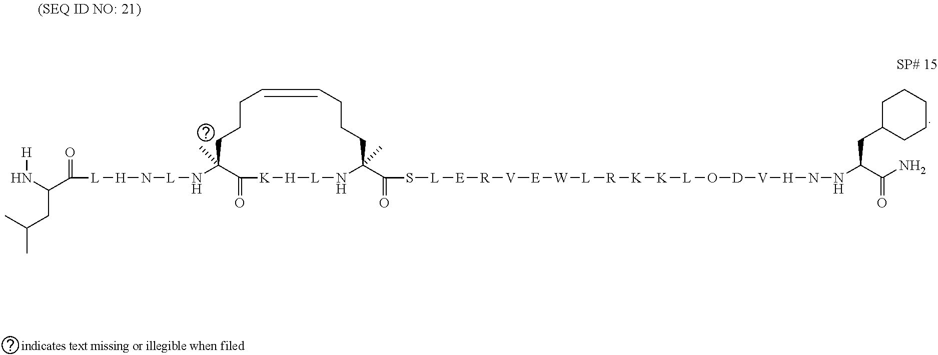

In one aspect, a composition is provided comprising a peptidomimetic macrocycle, wherein the peptidomimetic macrocycle is

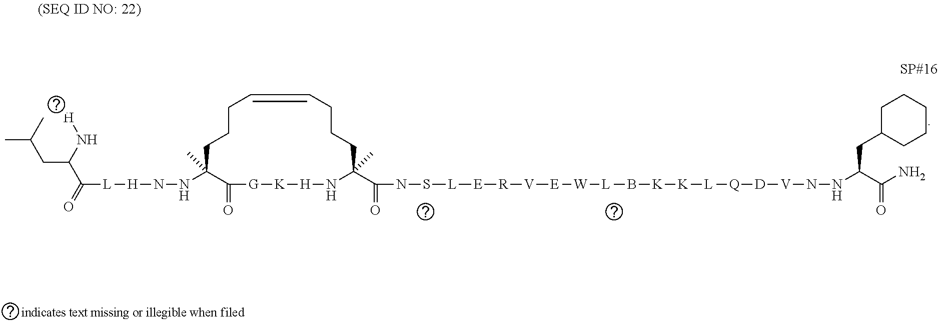

In one aspect, a composition is provided comprising a peptidomimetic macrocycle, wherein the peptidomimetic macrocycle is

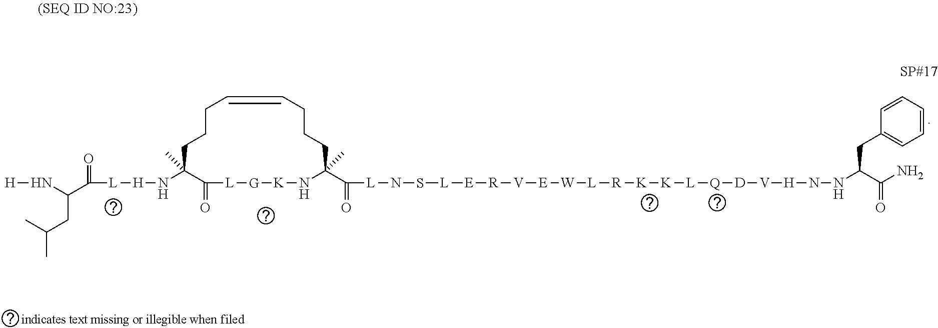

In one aspect, a composition is provided comprising a peptidomimetic macrocycle, wherein the peptidomimetic macrocycle is

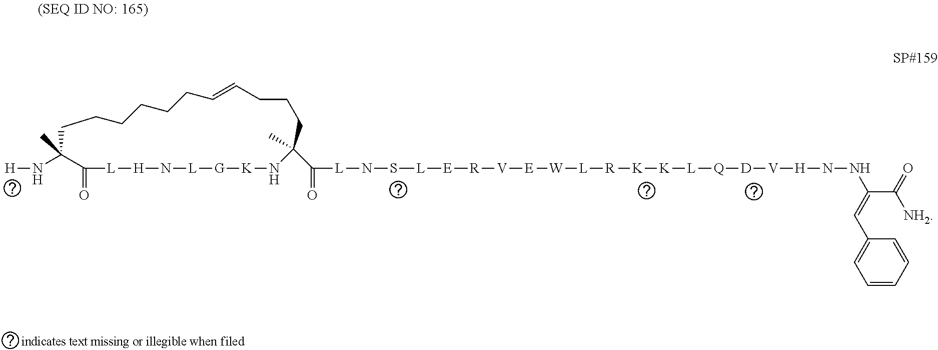

In one aspect, a composition is provided comprising a peptidomimetic macrocycle, wherein the peptidomimetic macrocycle is

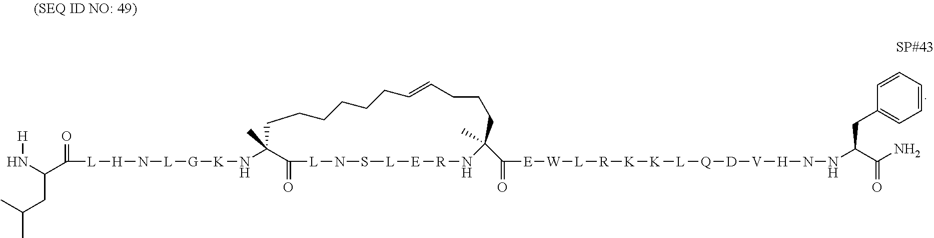

In one aspect, a composition is provided comprising a peptidomimetic macrocycle, wherein the peptidomimetic macrocycle is

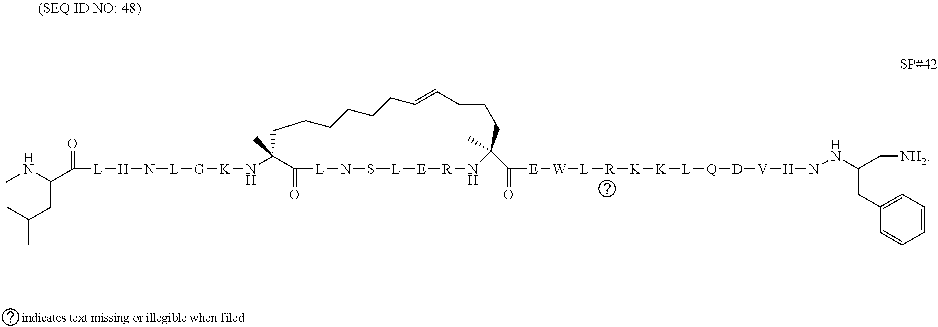

In one aspect, a composition is provided comprising a peptidomimetic macrocycle, wherein the peptidomimetic macrocycle is

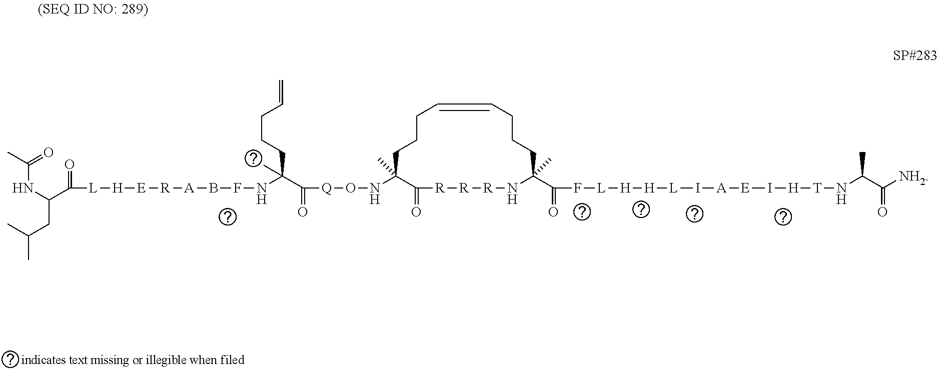

In one aspect, a composition is provided comprising a peptidomimetic macrocycle, wherein the peptidomimetic macrocycle is

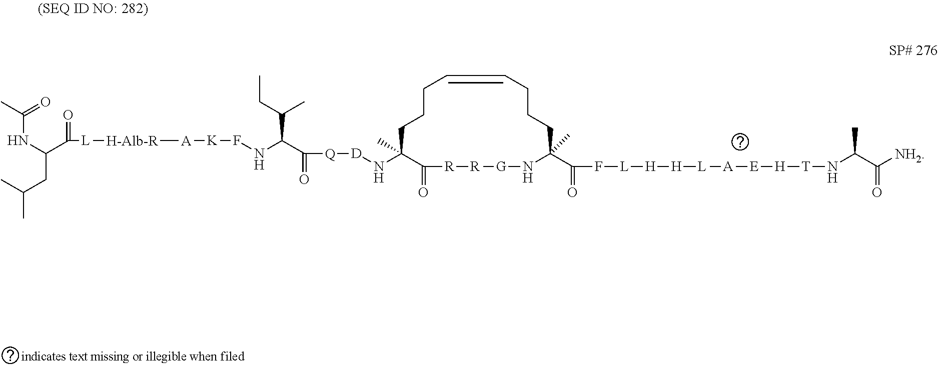

In one aspect, a composition is provided comprising a peptidomimetic macrocycle, wherein the peptidomimetic macrocycle is

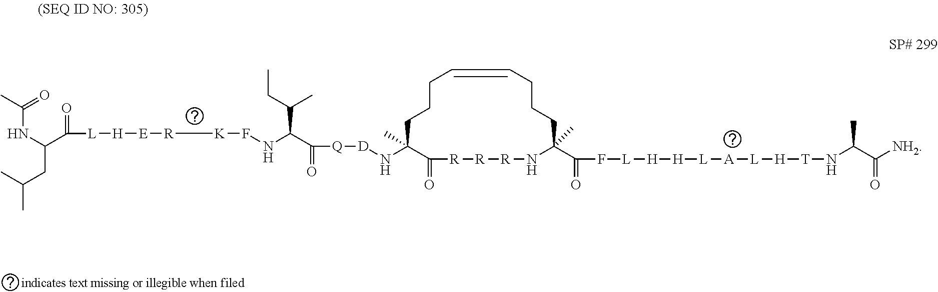

In one aspect, a composition is provided comprising a peptidomimetic macrocycle, wherein the peptidomimetic macrocycle is

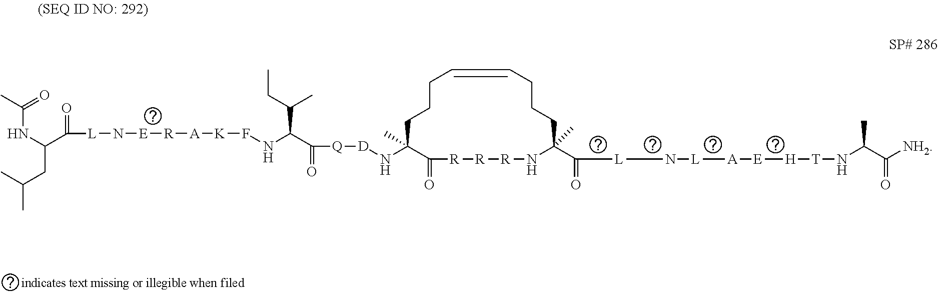

In one aspect, a composition is provided comprising a peptidomimetic macrocycle, wherein the peptidomimetic macrocycle is

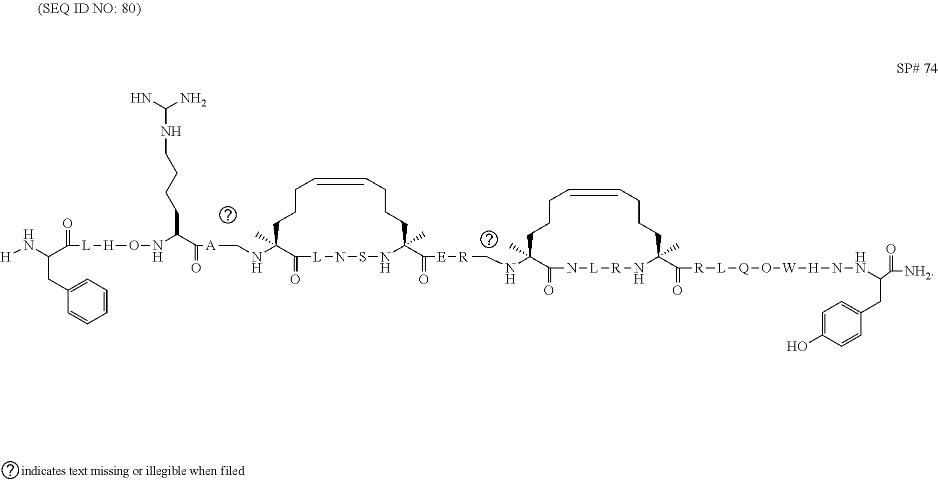

In one aspect, a composition is provided comprising a peptidomimetic macrocycle, wherein the peptidomimetic macrocycle is

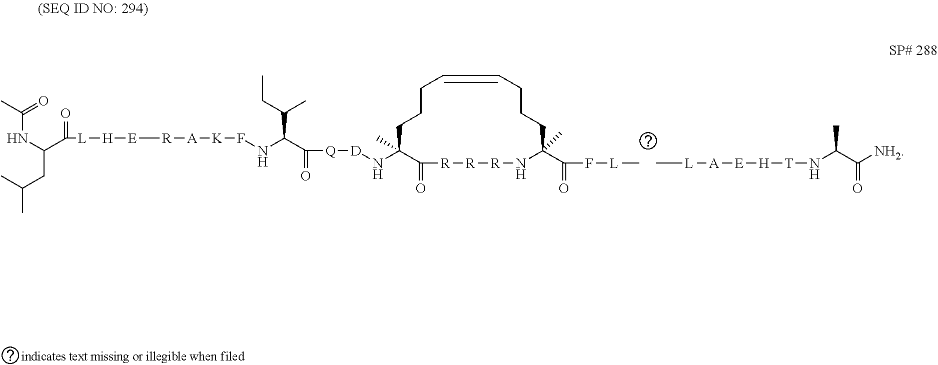

In one aspect, a composition is provided comprising a peptidomimetic macrocycle, wherein the peptidomimetic macrocycle is

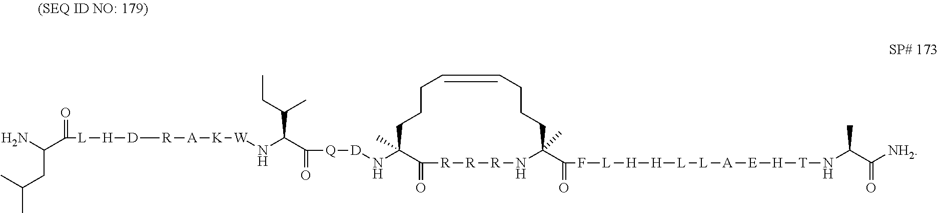

In one aspect, a composition is provided comprising a peptidomimetic macrocycle, wherein the peptidomimetic macrocycle is

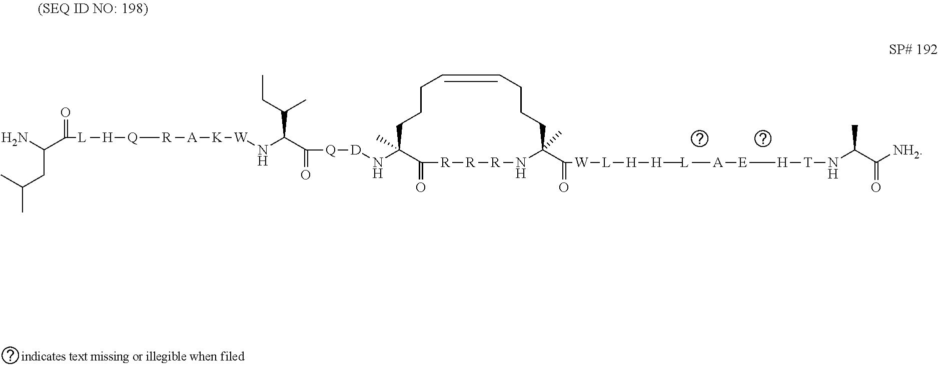

In one aspect, a composition is provided comprising a peptidomimetic macrocycle, wherein the peptidomimetic macrocycle is

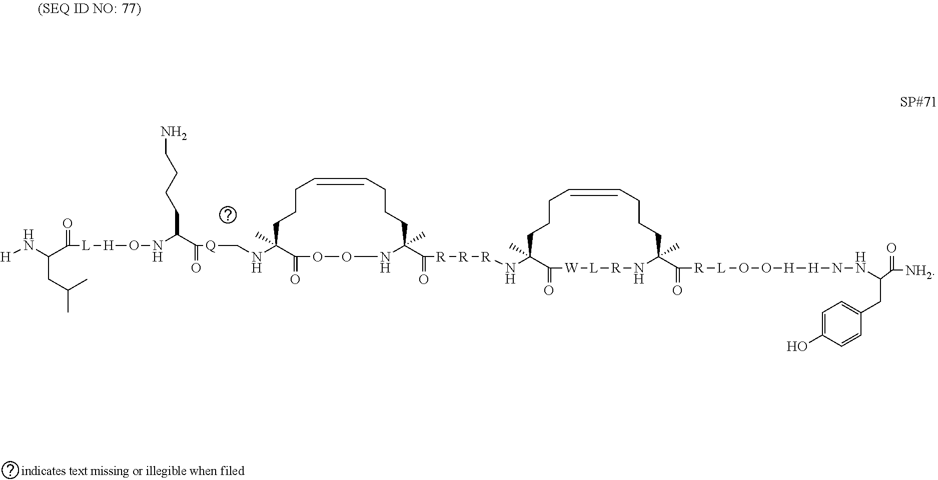

In one aspect, a composition is provided comprising a peptidomimetic macrocycle, wherein the peptidomimetic macrocycle is

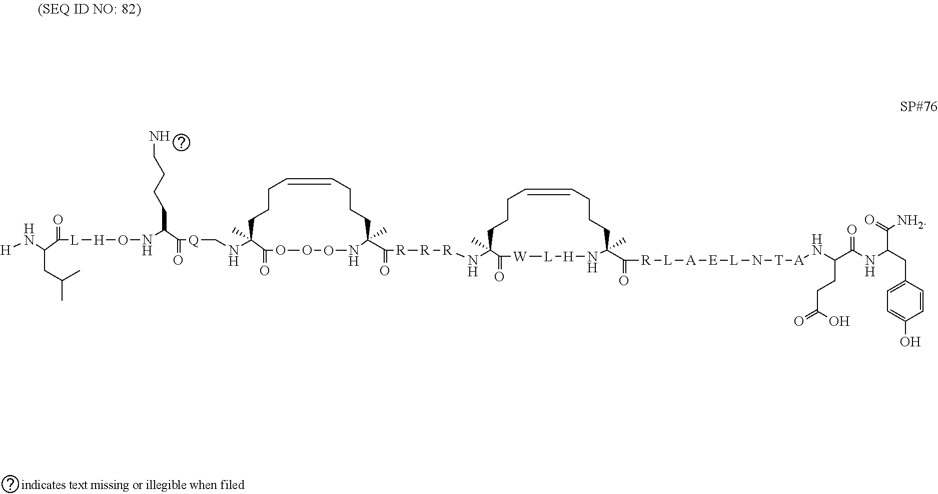

In one aspect, a composition is provided comprising a peptidomimetic macrocycle, wherein the peptidomimetic macrocycle is

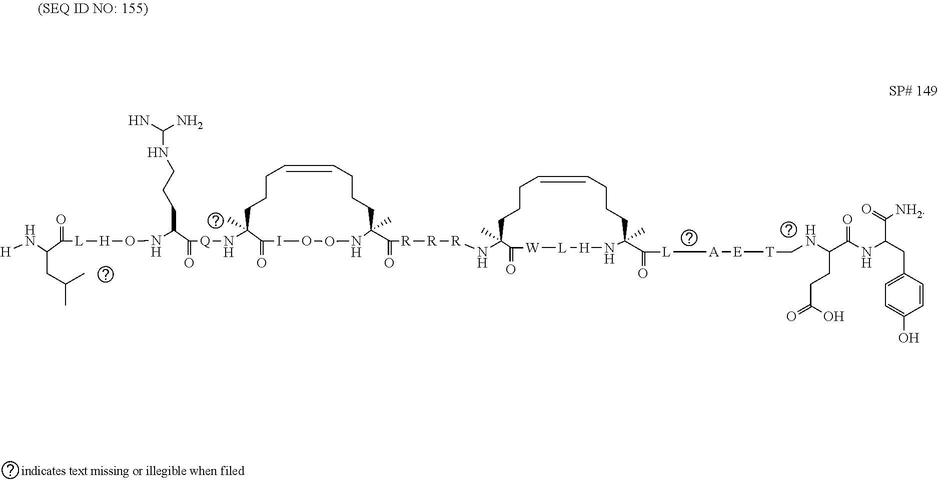

In one aspect, a composition is provided comprising a peptidomimetic macrocycle, wherein the peptidomimetic macrocycle is

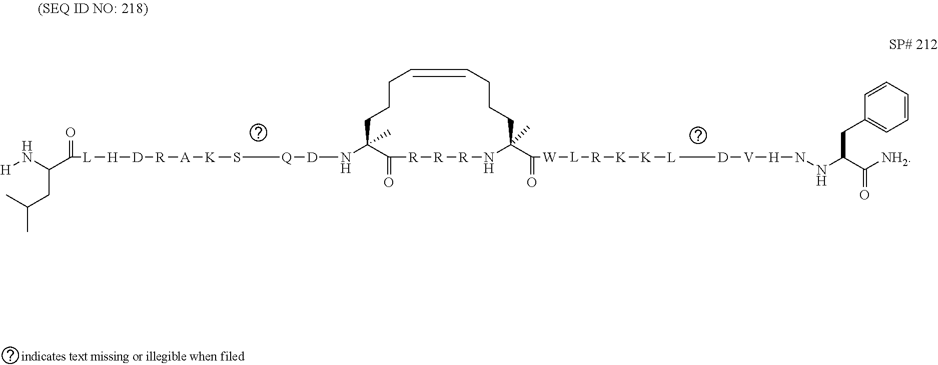

In one aspect, a composition is provided comprising a peptidomimetic macrocycle, wherein the peptidomimetic macrocycle is

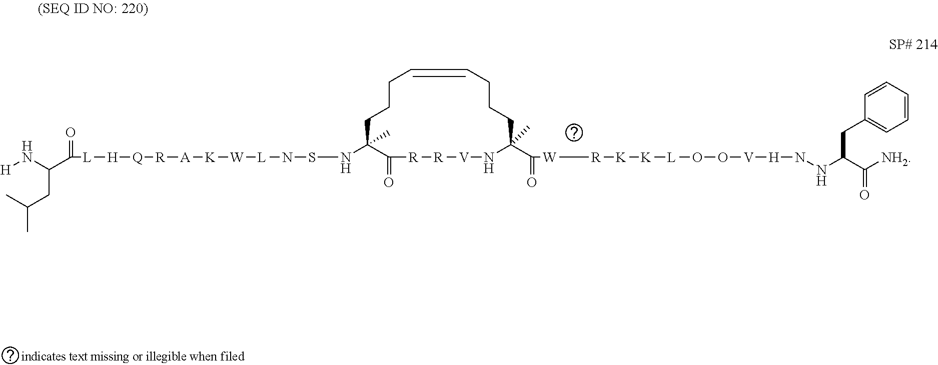

In one aspect, a composition is provided comprising a peptidomimetic macrocycle, wherein the peptidomimetic macrocycle is

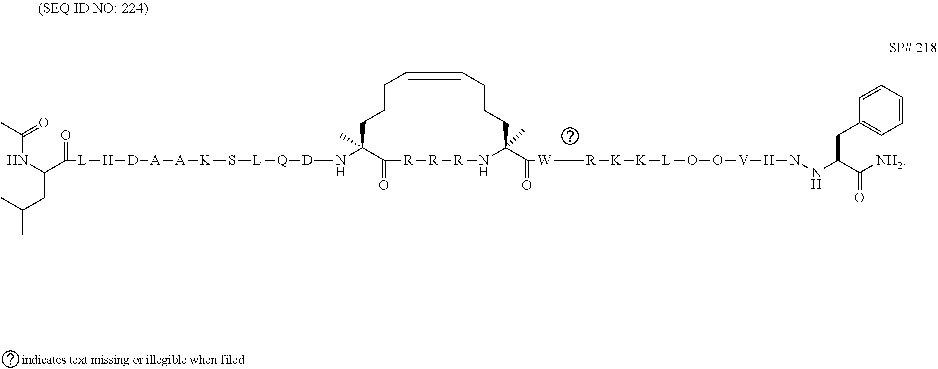

In one aspect, a composition is provided comprising a peptidomimetic macrocycle, wherein the peptidomimetic macrocycle is

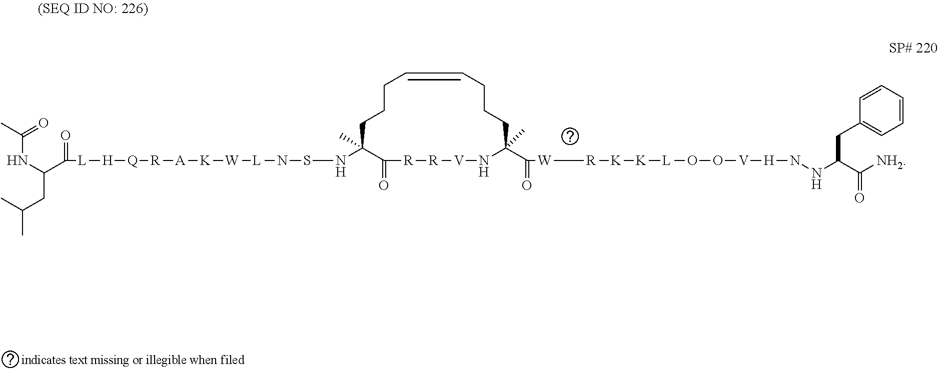

In one aspect, a composition is provided comprising a peptidomimetic macrocycle, wherein the peptidomimetic macrocycle is

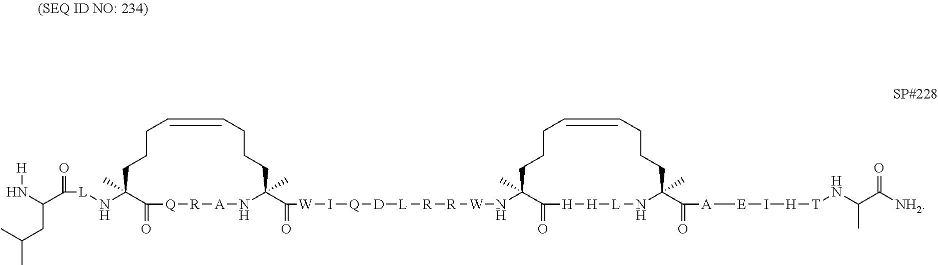

In one aspect, a composition is provided comprising a peptidomimetic macrocycle, wherein the peptidomimetic macrocycle is

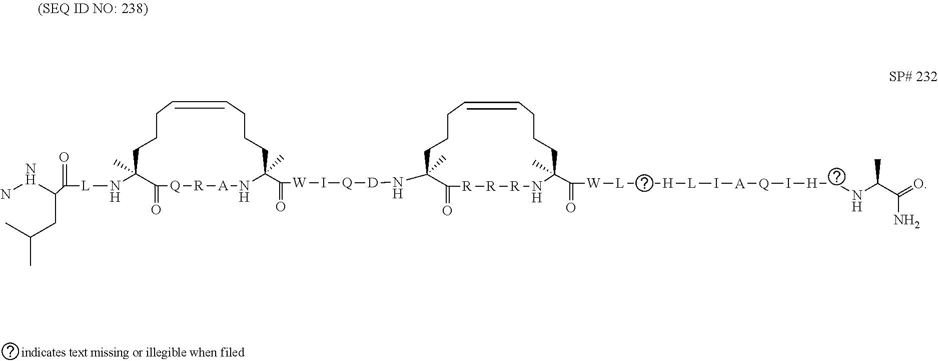

In one aspect, a composition is provided comprising a peptidomimetic macrocycle, wherein the peptidomimetic macrocycle is

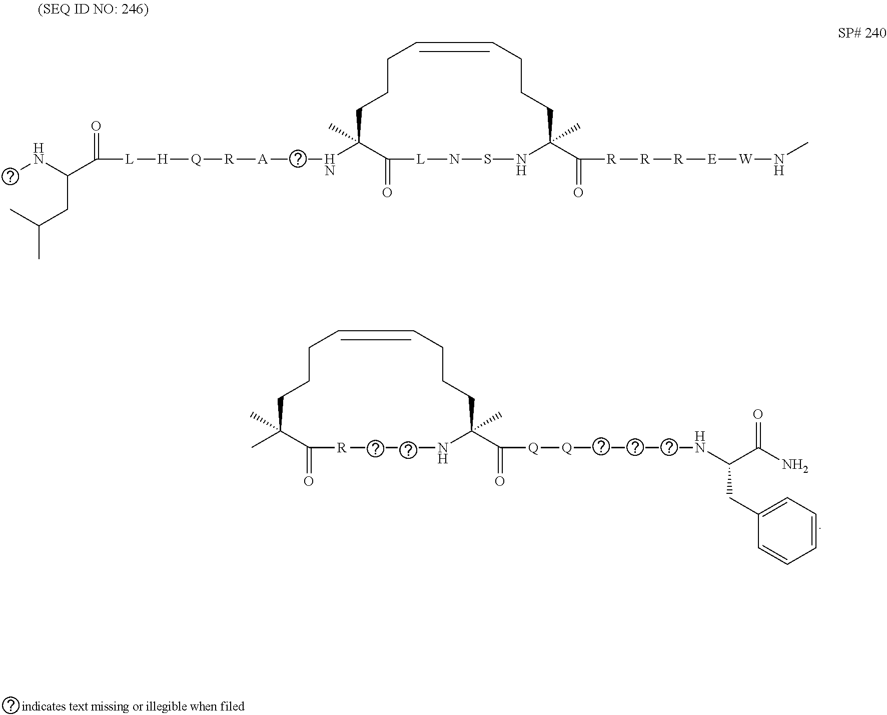

In one aspect, a composition is provided comprising a peptidomimetic macrocycle, wherein the peptidomimetic macrocycle is

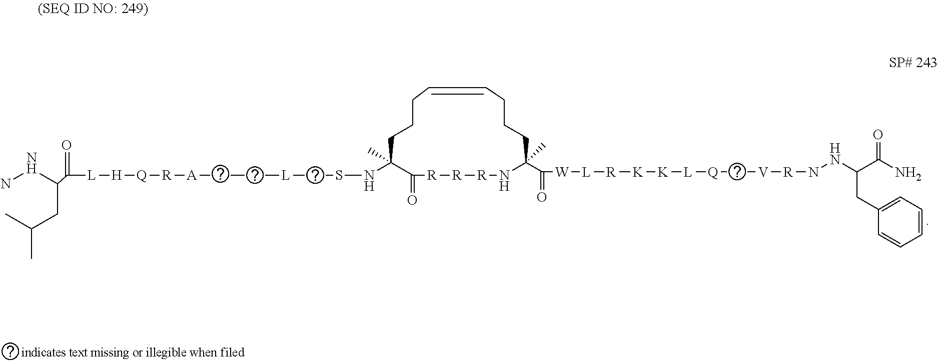

In one aspect, a composition is provided comprising a peptidomimetic macrocycle, wherein the peptidomimetic macrocycle is

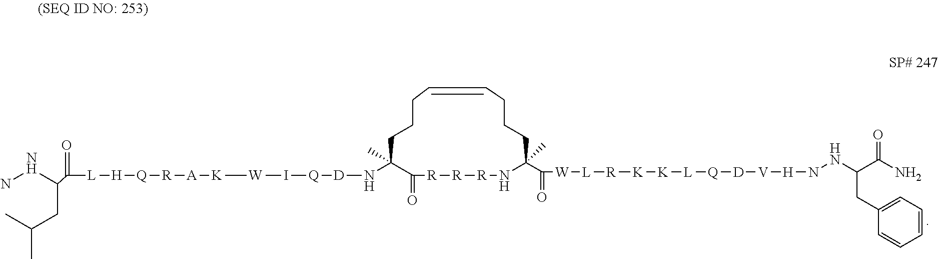

In one aspect, a composition is provided comprising a peptidomimetic macrocycle, wherein the peptidomimetic macrocycle is

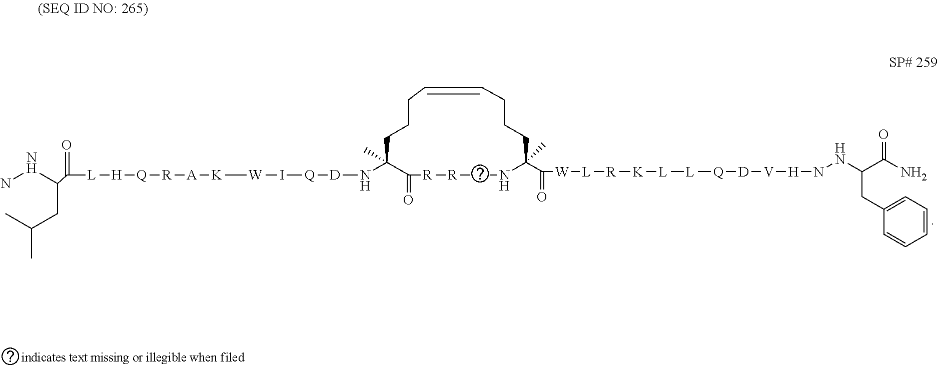

In one aspect, a composition is provided comprising a peptidomimetic macrocycle, wherein the peptidomimetic macrocycle is

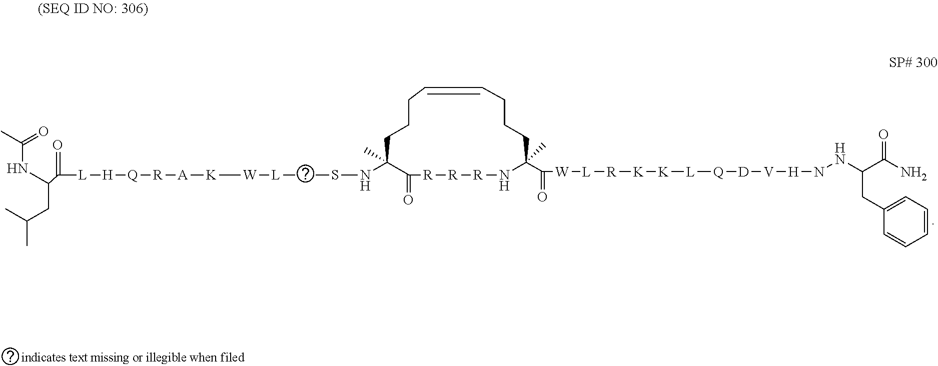

In one aspect, a composition is provided comprising a peptidomimetic macrocycle, wherein the peptidomimetic macrocycle is

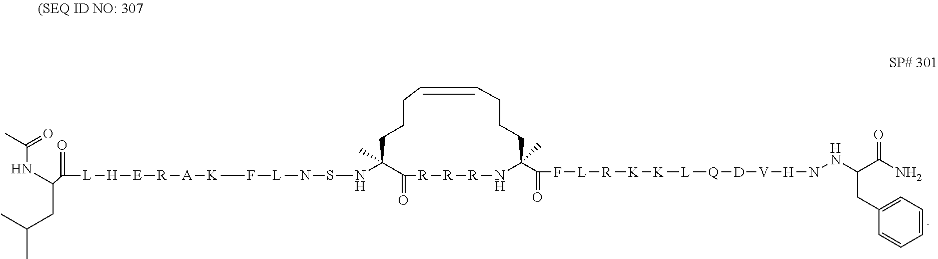

In one aspect, a composition is provided comprising a peptidomimetic macrocycle, wherein the peptidomimetic macrocycle is

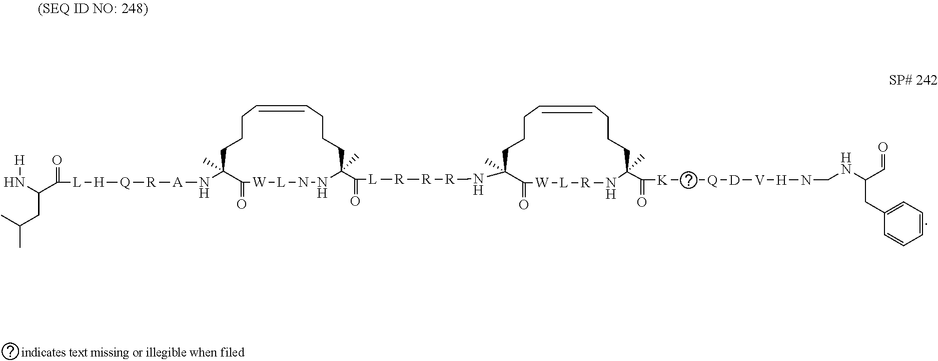

In one aspect, a composition is provided comprising a peptidomimetic macrocycle, wherein the peptidomimetic macrocycle is

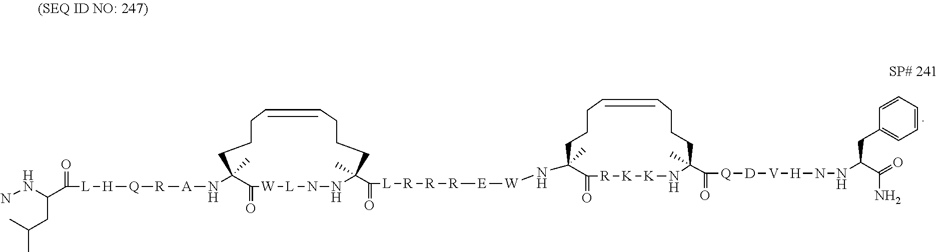

In one aspect, a composition is provided comprising a peptidomimetic macrocycle, wherein the peptidomimetic macrocycle is

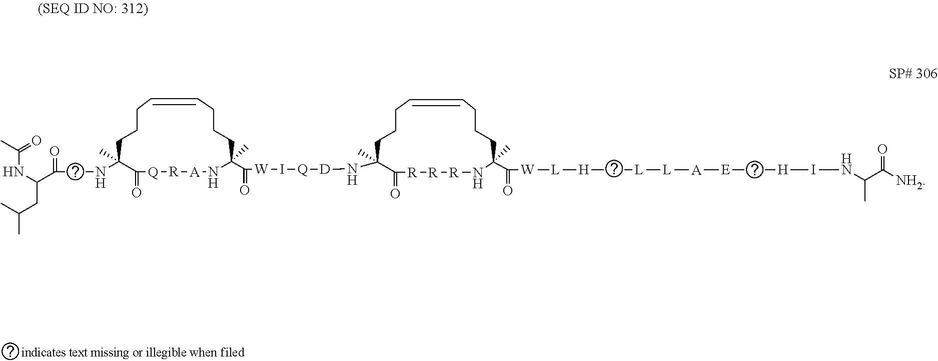

In one aspect, a composition is provided comprising a peptidomimetic macrocycle, wherein the peptidomimetic macrocycle is

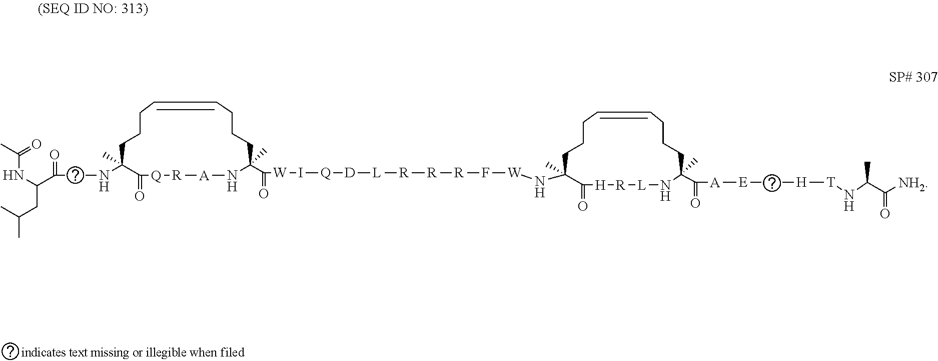

In one aspect, a composition is provided comprising a peptidomimetic macrocycle, wherein the peptidomimetic macrocycle is

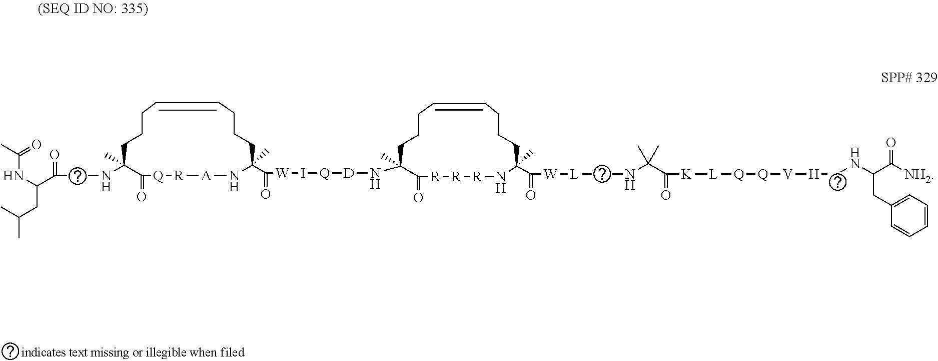

In one aspect, a composition is provided comprising a peptidomimetic macrocycle, wherein the peptidomimetic macrocycle is

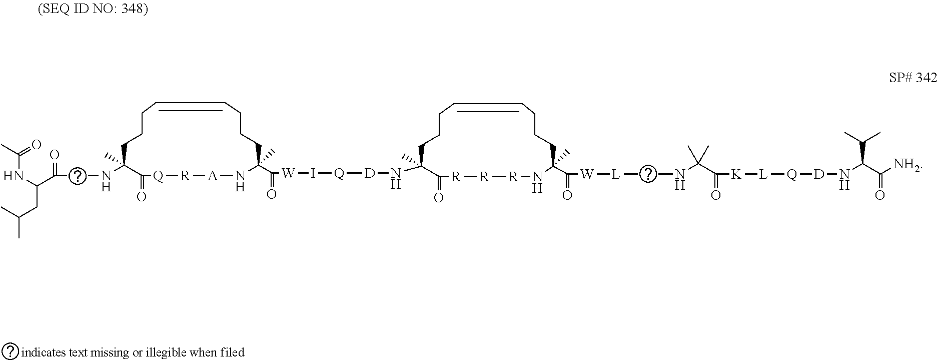

In one aspect, a composition is provided comprising a peptidomimetic macrocycle, wherein the peptidomimetic macrocycle is

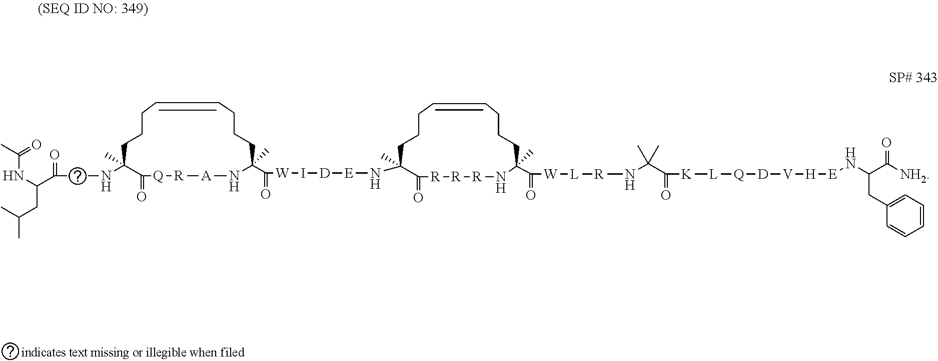

In one aspect, a composition is provided comprising a peptidomimetic macrocycle, wherein the peptidomimetic macrocycle is

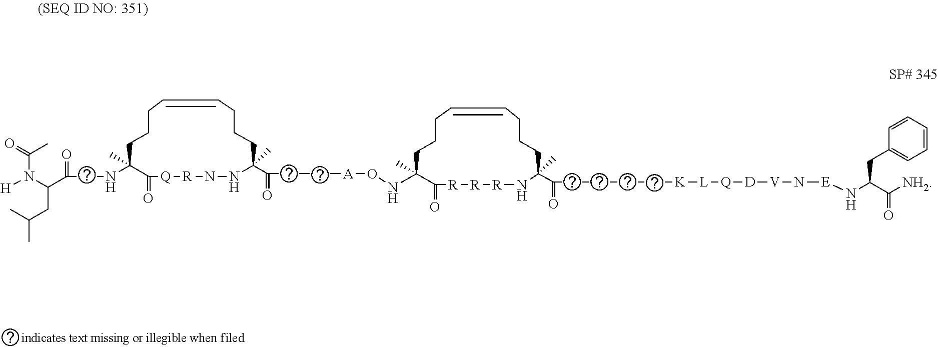

In one aspect, a composition is provided comprising a peptidomimetic macrocycle, wherein the peptidomimetic macrocycle is

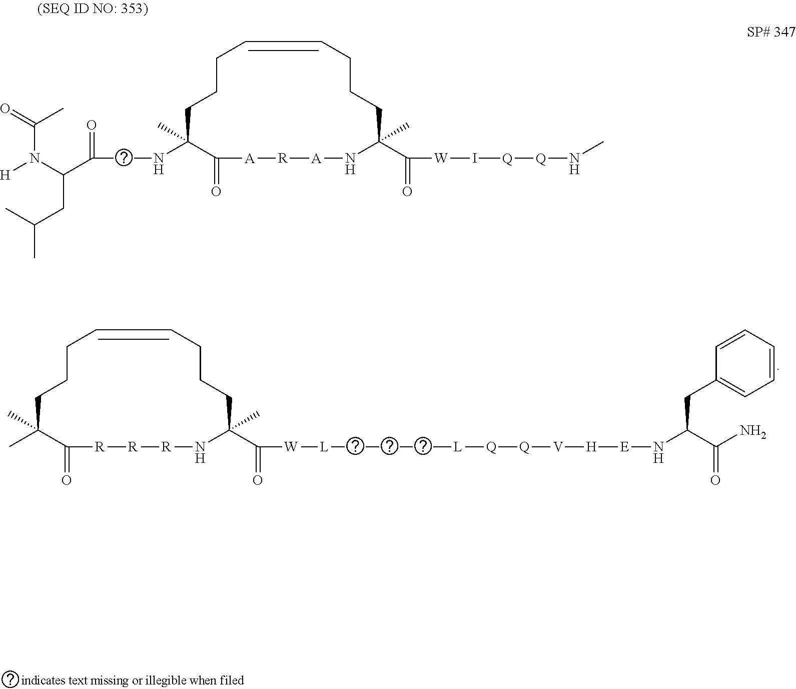

In one aspect, a composition is provided comprising a peptidomimetic macrocycle, wherein the peptidomimetic macrocycle is

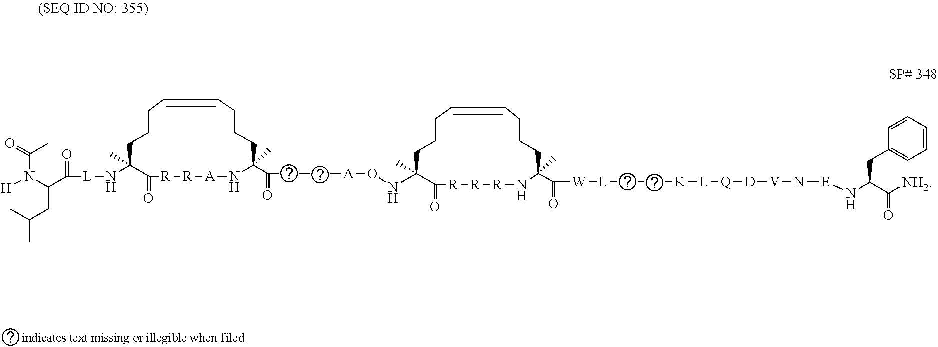

In one aspect, a composition is provided comprising a peptidomimetic macrocycle, wherein the peptidomimetic macrocycle is

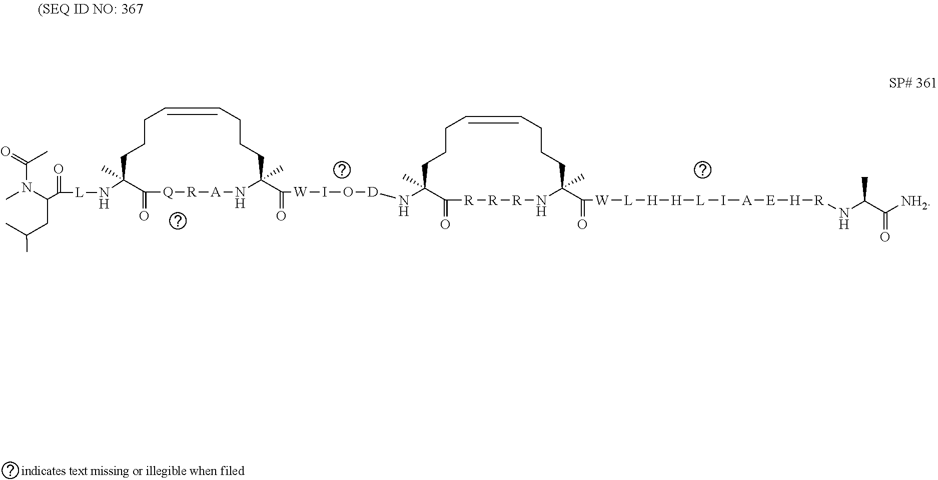

In one aspect, a composition is provided comprising a peptidomimetic macrocycle, wherein the peptidomimetic macrocycle is

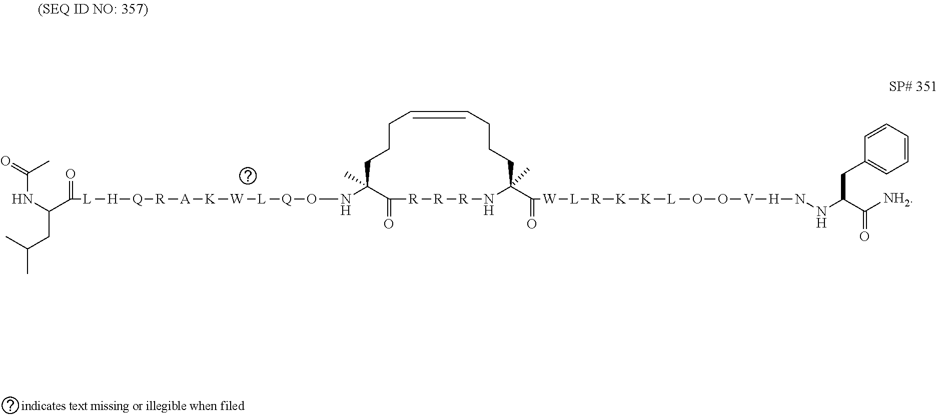

In one aspect, a composition is provided comprising a peptidomimetic macrocycle, wherein the peptidomimetic macrocycle is

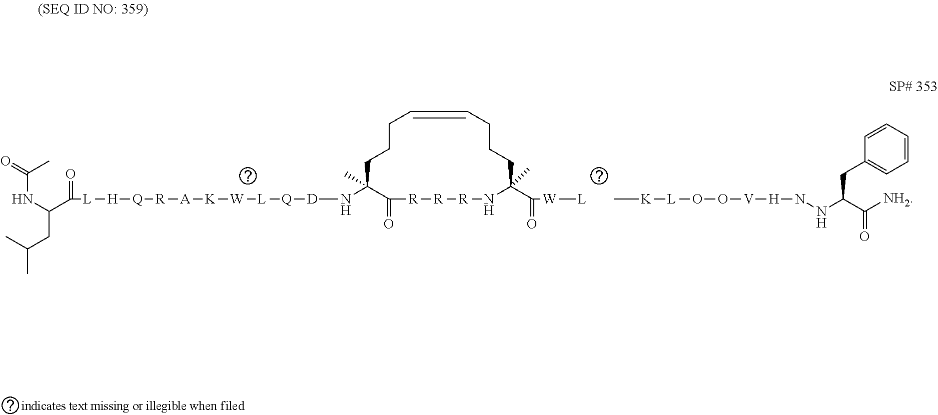

In one aspect, a composition is provided comprising a peptidomimetic macrocycle, wherein the peptidomimetic macrocycle is

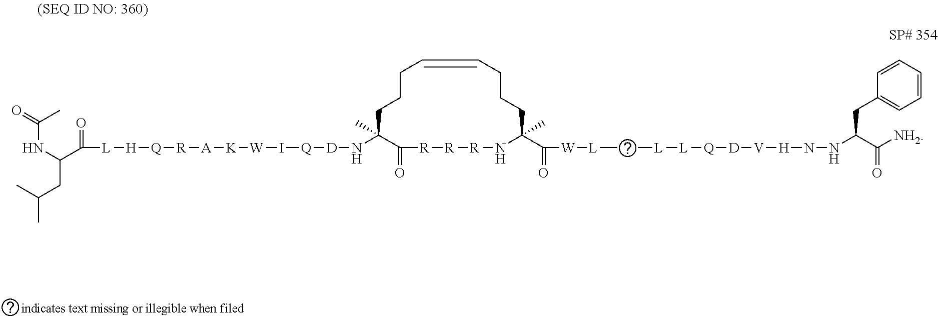

In one aspect, a composition is provided comprising a peptidomimetic macrocycle, wherein the peptidomimetic macrocycle is

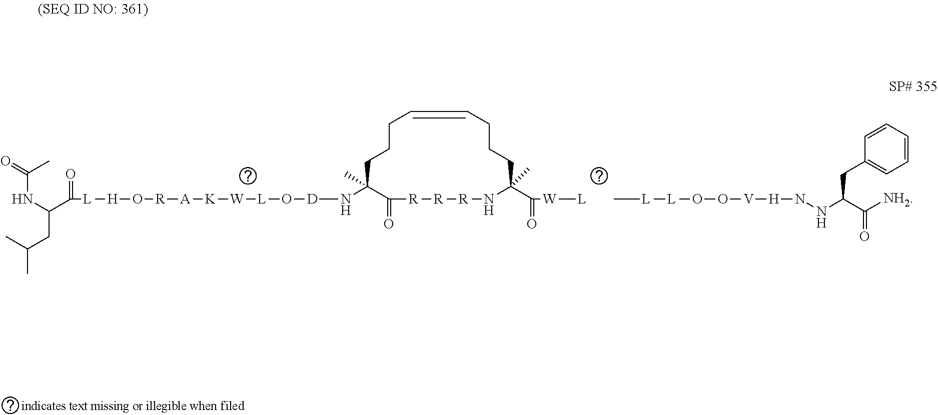

In one aspect, a composition is provided comprising a peptidomimetic macrocycle, wherein the peptidomimetic macrocycle is

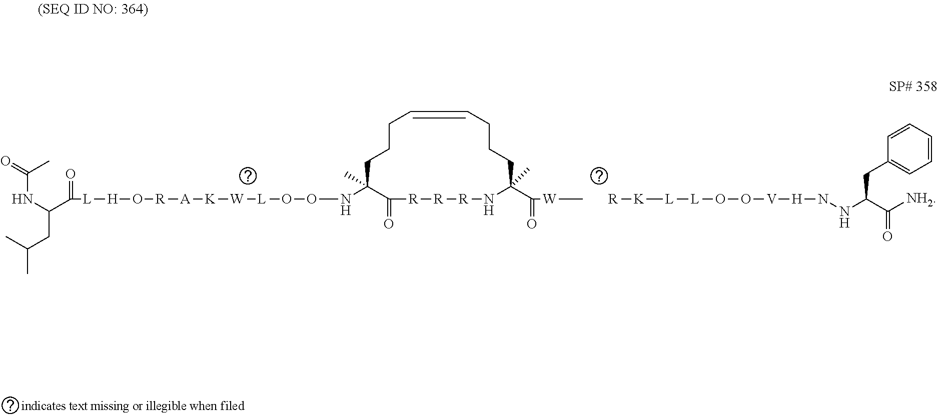

In one aspect, a composition is provided comprising a peptidomimetic macrocycle, wherein the peptidomimetic macrocycle is

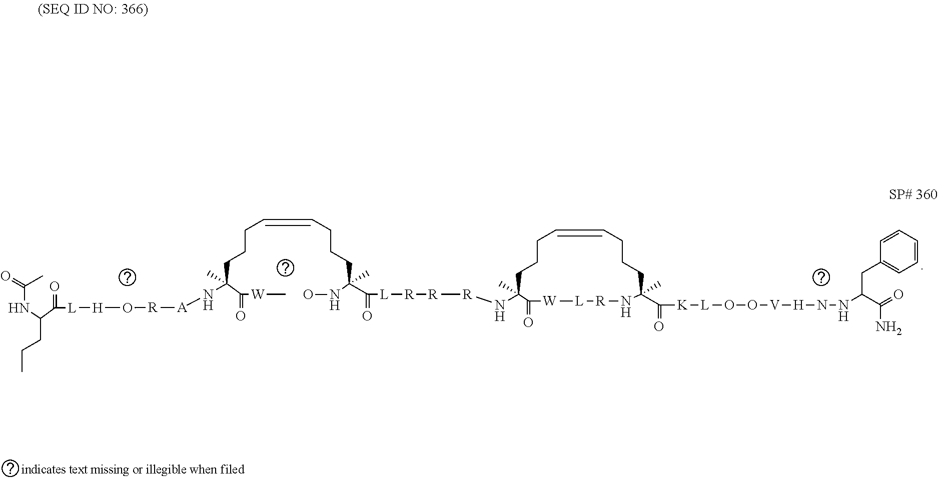

In one aspect, a composition is provided comprising a peptidomimetic macrocycle, wherein the peptidomimetic macrocycle is

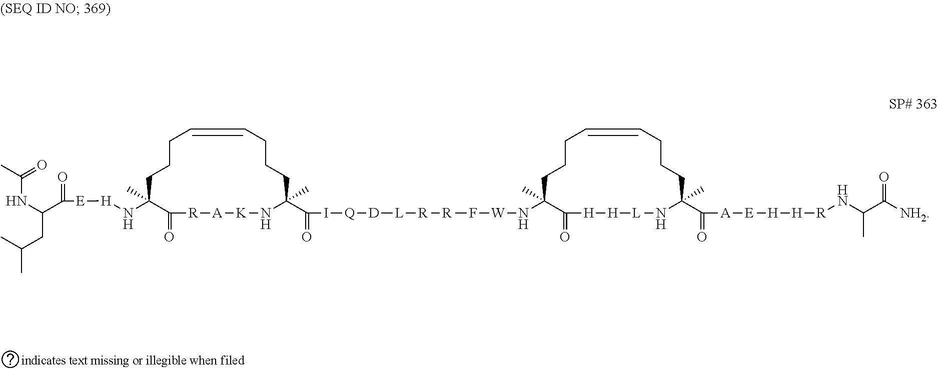

In one aspect, a composition is provided comprising a peptidomimetic macrocycle, wherein the peptidomimetic macrocycle is

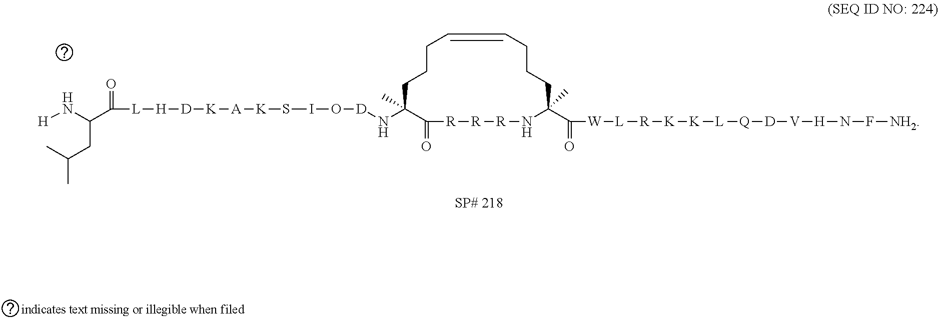

In one aspect, a composition is provided comprising a peptidomimetic macrocycle, wherein the peptidomimetic macrocycle is

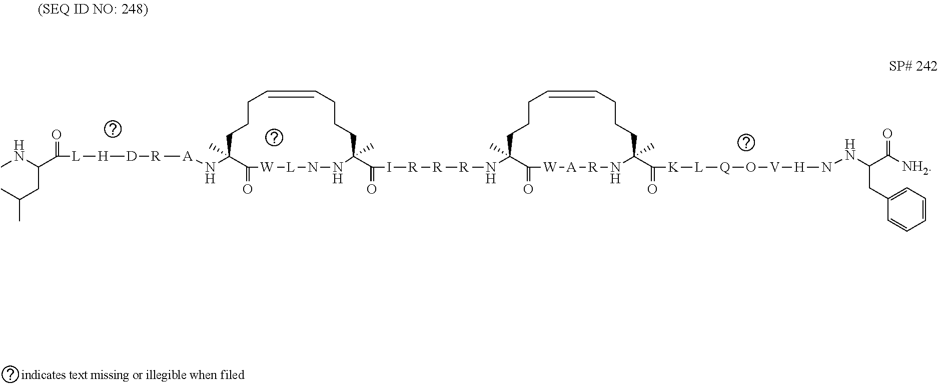

In one aspect, a composition is provided comprising a peptidomimetic macrocycle, wherein the peptidomimetic macrocycle is

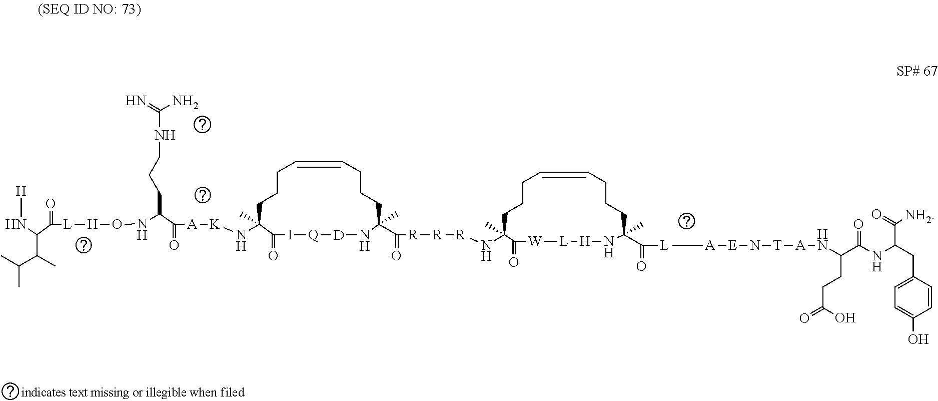

In one aspect, a composition is provided comprising a peptidomimetic macrocycle, wherein the peptidomimetic macrocycle is

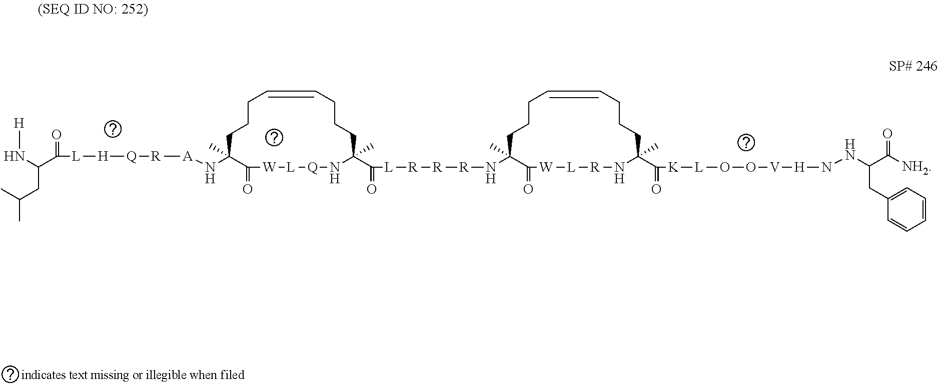

In one aspect, a pharmaceutical composition is provided comprising a peptidomimetic macrocycle described herein and a pharmaceutically acceptable excipient.

In one aspect, a method is disclosed for use of a peptidomimetic macrocycle or pharmaceutical composition provided herein in the treatment of a disease.

In one aspect, a method is disclosed for use of a peptidomimetic macrocycle or pharmaceutical composition provided herein in the manufacture of a medicament for treatment of a disease.

In one aspect, a method is disclosed, wherein the method is a method of preparing a composition comprising a peptidomimetic macrocycle of Formula (IV)

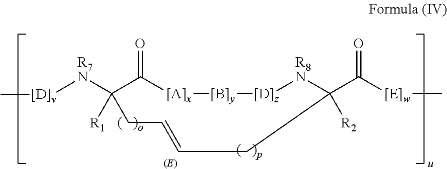

comprising an amino acid sequence that has about 60%, 65%, 70%, 75%, 80%, 85%, 90%, 95%, 96%, 97%, 98%, 99% or 100% sequence identity to a sequence of Table 1a, 1b, 2a, or 2b, wherein the peptidomimetic macrocycle comprises at least two non-natural amino acids connected by a macrocycle-forming linker, the method comprising treating a compound of Formula (V)

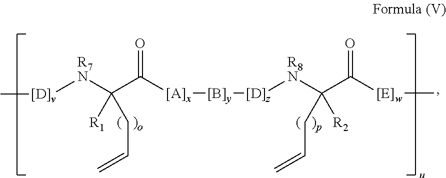

with a catalyst to result in the compound of Formula (IV)
wherein in the compound(s) of Formulae (IV) and (V) each A, C, D, and E is independently an amino acid; each B is independently an amino acid,

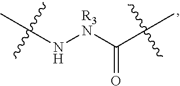

[—NH-L₃-CO—], [—NH-L₃-SO₂—], or [—NH-L₃-]; each R₁ and R₂ are independently —H, alkyl, alkenyl, alkynyl, arylalkyl, cycloalkyl, cycloalkylalkyl, heteroalkyl, or heterocycloalkyl, unsubstituted or substituted with halogen; or at least one of R₁ and R₂ forms a macrocycle-forming linker L′ connected to the alpha position of one of the D or E amino acids; each R₃ is independently —H, alkyl, alkenyl, alkynyl, arylalkyl, heteroalkyl, cycloalkyl, heterocycloalkyl, cycloalkylalkyl, cycloaryl, or heterocycloaryl, optionally substituted with R₅; each L′ is independently a macrocycle-forming linker of the formula -L₁-L₂-; each L₁, L₂ and L₃ are independently alkylene, alkenylene, alkynylene, heteroalkylene, cycloalkylene, heterocycloalkylene, cycloarylene, heterocycloarylene, or [—R₄—K—R₄′—]ₙ, each being optionally substituted with R₅; each R₄ and R_{4′} is independently alkylene, alkenylene, alkynylene, heteroalkylene, cycloalkylene, heterocycloalkylene, arylene, or heteroarylene; each K is independently O, S, SO, SO₂, CO, CO₂ or CONR₃; each R₅ is independently halogen, alkyl, —OR₆, —N(R₆)₂, —SR₆, —SOR₆, —SO₂R₆, —CO₂R₆, a fluorescent moiety, a radioisotope or a therapeutic agent; each R₆ is independently —H, alkyl, alkenyl, alkynyl, arylalkyl, cycloalkylalkyl, heterocycloalkyl, a fluorescent moiety, a radioisotope or a therapeutic agent; each R₇ is independently —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue; each R₈ is independently —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue; each v and w is independently an integer from 1-1000; u is an integer from 1-10; each x, y and z is independently an integer from 0-10; each n is independently an integer from 1-5; each o is independently an integer from 1-15; each p is independently an integer from 1-15; “(E)” indicates a trans double bond; and one or more of the amino acids A, C and/or B when B is an amino acid, present in the compounds of Formulae (IV) and (V), has a side chain bearing a protecting group.

In some embodiments, the protecting group is a nitrogen atom protecting group. In some embodiments, the protecting group is a Boc group. In some embodiments, the side chain of the amino acid bearing the protecting group comprises a protected indole. In some embodiments, the amino acid bearing the protecting group on its side chain is tryptophan (W) that is protected by the protecting group on its indole nitrogen. In some embodiments, the amino acid bearing the protecting group on its side chain is tryptophan (W) that is protected on its indole nitrogen by a Boc group.

In some embodiments, after the step of contacting the compound of Formula (V) with catalyst the compound of Formula (IV) is obtained in equal or higher amounts than a corresponding compound which is a Z isomer. In some embodiments, after the step of contacting the compound of Formula (V) with catalyst the compound of Formula (IV) is obtained in a 2, 3, 4, 5, 6, 7, 8, 9, or 10-fold higher amount than the corresponding compound which is a Z isomer. In some embodiments, the catalyst is a ruthenium catalyst.

In some embodiments, the method further comprises the step of treating the compounds of Formula (IV) with a reducing agent or an oxidizing agent. In some embodiments, the compound of Formula (V) is attached to a solid support. In some embodiments, the compound of Formula (V) is not attached to a solid support. In some embodiments, the method further comprises removing the protecting group(s) from the compounds of Formula (IV). In some embodiments, the ring closing metathesis is conducted at a temperature ranging from about 20° C. to about 80° C.

In one aspect, a method is disclosed for treating a condition characterized by increased or decreased activity or production of PTH or PTHrP in a subject in need thereof, comprising administering to the subject an effective amount of a peptidomimetic macrocycle or pharmaceutical composition provided herein. In one aspect, a method is disclosed for treating a condition characterized by increased or decreased activity or production of PTH or PTHrP in a subject in need thereof, comprising administering to the subject an effective amount of a composition comprising a peptidomimetic macrocycle provided herein. In some embodiments, the condition is hypoparathyroidism. In some embodiments, the condition is hyperparathyroidism or hypercalcemia. In some embodiments, the condition is primary hyperparathyroidism. In some embodiments, the subject suffers from a parathyroid adenoma, parathyroid hyperplasia, or a parathyroid carcinoma. In some embodiments, the parathyroid carcinoma is inoperable parathyroid tumor. In some embodiments, the inoperable parathyroid tumor is metaphyseal chondrodysplasia. In some embodiments, the subject suffers from a multiple endocrine neoplasia or familial hyperparathyroidism. In some embodiments, the condition is secondary hyperparathyroidism. In some embodiments, the subject suffers from a renal disorder or vitamin D deficiency. In some embodiments, the renal disorder is chronic kidney disease. In some embodiments, the chronic kidney disease is in stage 1, 2, 3 or 4. In some embodiments, the subject is undergoing dialysis. In some embodiments, the condition is tertiary hyperparathyroidism.

In one aspect, a method is disclosed for decreasing the activity of PTH or PTHrP in a subject in need thereof, comprising administering to the subject an effective amount of a peptidomimetic macrocycle or pharmaceutical composition provided herein. In one aspect, a method is disclosed for decreasing the activity of PTH or PTHrP in a subject in need thereof, comprising administering to the subject an effective amount of a peptidomimetic macrocycle or pharmaceutical composition provided herein. In one aspect, a method is disclosed for treating a condition characterized by a decrease in adipose tissue or insufficient adipose tissue or a decrease in skeletal muscle tissue or insufficient skeletal muscle tissue comprising administering to the subject an effective amount of a peptidomimetic macrocycle or pharmaceutical composition provided herein. In one aspect, a method is disclosed for treating a condition characterized by a decrease in adipose tissue or insufficient adipose tissue or a decrease in skeletal muscle tissue or insufficient skeletal muscle tissue comprising administering to the subject an effective amount of a peptidomimetic macrocycle or pharmaceutical composition provided herein. In some embodiments, the condition is cachexia. In some embodiments, the condition is cancer cachexia. In some embodiments, the condition is an increased resting energy expenditure level. In some embodiments, the condition is an increased thermogenesis by brown fat.

In one aspect, a method is disclosed for treating a condition of skin or hair, comprising administering to the subject an effective amount of a peptidomimetic macrocycle or pharmaceutical composition provided herein. In one aspect, a method is disclosed for treating a condition of skin or hair, comprising administering to the subject an effective amount of a composition comprising a peptidomimetic macrocycle described herein. In some embodiments, the condition is insufficient hair growth. In some embodiments, the condition is psoriasis.

In one aspect, a method is disclosed for treating a condition characterized by a decrease in bone mass or insufficient bone mass in a subject, comprising administering to the subject an effective amount of a composition comprising a peptidomimetic macrocycle described herein. In one aspect, a method is disclosed for treating a condition characterized by a decrease in bone mass or insufficient bone mass in a subject, comprising administering to the subject an effective amount of a composition comprising a peptidomimetic macrocycle described herein. In some embodiments, the condition is osteoporosis. In some embodiments, the condition is osteopenia.

In some embodiments, the peptidomimetic macrocycle is administered parenterally. In some embodiments, the peptidomimetic macrocycle is administered subcutaneously. In some embodiments, the peptidomimetic macrocycle is administered intravenously.

In some embodiments, the administering is no more frequently than once daily, no more frequently than every other day, no more frequently than three times weekly, no more frequently than twice weekly, no more frequently than weekly, or no more frequently than every other week. In some embodiments, the administering is no more frequently than three times weekly. In some embodiments, the administering is no more frequently than weekly, for example once weekly.

### INCORPORATION BY REFERENCE

All publications, patents, and patent applications mentioned in this specification are herein incorporated by reference in their entirety for all purposes, to the same extent as if each individual publication, patent, or patent application was specifically and individually indicated to be incorporated by reference.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 depicts an exemplary assay comparing the effects of an exemplary compound described herein with previously reported PTH antagonists on cAMP production in UMR-106 rat osteosarcoma cells. The results indicate that the exemplary compound described herein shows better in vitro anti-PTH activity than any published antagonist.

FIG. 2 depicts an exemplary assay demonstrating the effects on serum calcium levels in normal rats after 20 mL/kg IV administration of an exemplary compound described herein at 3 mg/kg or vehicle. The data shows a durable drop in calcium (greater than vehicle-related hemodilution) observed after dosing in normal rats.

FIG. 3A depicts an exemplary graph of the antagonism activity of BIM-44002 on PTH2R.

FIG. 3B depicts an exemplary graph of the antagonism activity of SP-67 macrocycle on PTH2R.

FIG. 4 shows an exemplary assay comparing the effects of several exemplary PTH antagonists described herein on cAMP production in SaOS-2 human osteosarcoma cells treated with 10 nM PTH (1-34).

FIG. 5 shows an exemplary assay comparing the effects of an exemplary PTH antagonist described herein on cAMP production in SaOS-2 human osteosarcoma cells treated with native hormones PTH (1-34) and PTH (1-84) and PTHrP.

FIG. 6 shows an exemplary assay comparing the effects of several exemplary PTH antagonists described herein on cAMP production in SaOS-2 human osteosarcoma cells treated with 10 nM PTH (1-34).

FIG. 7 shows an exemplary binding constant (K_{B}) determination by Schild analysis.

FIG. 8 shows that an exemplary compound described herein reduces PTH-induced calcium levels in a rat hyperparathyroidism model (thyroparathyroidectomized rats). The compound reduced PTH-induced calcium rise over the course of treatment, demonstrating PTH-specific, on-mechanism in vivo activity.

FIG. 9A shows that an exemplary compound described herein reduces total calcium levels over time in the rat hyperparathyroidism model described in FIG. 8.

FIG. 9B shows that an exemplary compound described herein reduces ionized calcium levels over time in the rat hyperparathyroidism model described in FIG. 8.

FIG. 10 shows an exemplary effect of staple position on potency of macrocyclic PTH compounds described herein. Figure discloses SEQ ID NO: 446.

FIG. 11A depicts an exemplary graph showing that cells have negligent differences in cAMP production in the absence or presence of 1 μM of an exemplary compound described herein (SP-67) and the indicated ligands.

FIG. 11B depicts an exemplary graph showing the antagonistic specificity an exemplary compound described herein (SP-67) for the indicated ligands.

FIG. 12A depicts an exemplary graph showing that cells have negligent differences in cAMP production in the absence or presence of 1 μM of an exemplary compound described herein (SP-344) and the indicated ligands.

FIG. 12B depicts a graph showing the antagonistic specificity an exemplary compound described herein (SP-344) for the indicated ligands.

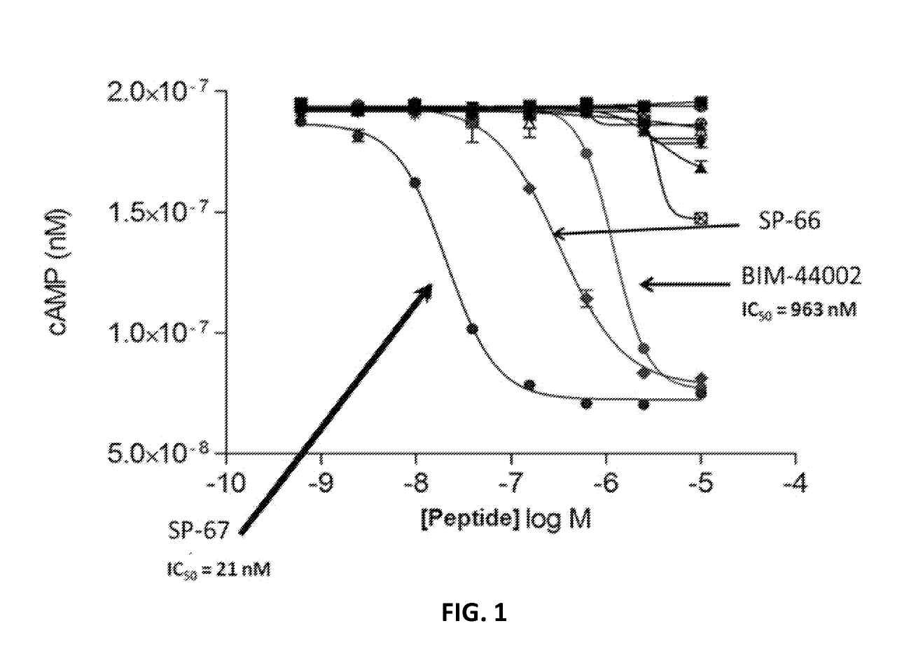

### DETAILED DESCRIPTION OF THE INVENTION

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, i.e. the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed. The term “about” has the meaning as commonly understood by one of ordinary skill in the art. In some embodiments, the term “about” refers to +10%. In some embodiments, the term “about” refers to +5%.

As used herein, the term “macrocycle” refers to a molecule having a chemical structure including a ring or cycle formed by at least 9 covalently bonded atoms.

As used herein, the term “peptidomimetic macrocycle” or “crosslinked polypeptide” refers to a compound comprising a plurality of amino acid residues joined by a plurality of peptide bonds and at least one macrocycle-forming linker which forms a macrocycle between a first naturally-occurring or non-naturally-occurring amino acid residue (or analog) and a second naturally-occurring or non-naturally-occurring amino acid residue (or analog) within the same molecule. Peptidomimetic macrocycles include embodiments where the macrocycle-forming linker connects the α-carbon of the first amino acid residue (or analog) to the α-carbon of the second amino acid residue (or analog). The peptidomimetic macrocycles optionally include one or more non-peptide bonds between one or more amino acid residues and/or amino acid analog residues, and optionally include one or more non-naturally-occurring amino acid residues or amino acid analog residues in addition to any which form the macrocycle. A “corresponding uncrosslinked polypeptide” when referred to in the context of a peptidomimetic macrocycle is understood to relate to a polypeptide of the same length as the macrocycle and comprising the equivalent natural amino acids of the wild-type sequence corresponding to the macrocycle.

As used herein, the term “stability” refers to the maintenance of a defined secondary structure in solution by a peptidomimetic macrocycle provided herein as measured by circular dichroism, NMR or another biophysical measure, or resistance to proteolytic degradation in vitro or in vivo. Non-limiting examples of secondary structures contemplated in this invention are α-helices, 3₁₀ helices, β-turns, and β-pleated sheets.

As used herein, the term “helical stability” refers to the maintenance of α helical structure by a peptidomimetic macrocycle provided herein as measured by circular dichroism or NMR. For example, in some embodiments, the peptidomimetic macrocycles provided herein exhibit at least a 1.25, 1.5, 1.75 or 2-fold increase in α-helicity as determined by circular dichroism compared to a corresponding uncrosslinked macrocycle.

The term “amino acid” refers to a molecule containing both an amino group and a carboxyl group. Suitable amino acids include, without limitation, both the D- and L-isomers of the naturally-occurring amino acids, as well as non-naturally occurring amino acids prepared by organic synthesis or other metabolic routes. The term amino acid, as used herein, includes without limitation, α-amino acids, natural amino acids, non-natural amino acids, and amino acid analogs.

The term “α-amino acid” refers to a molecule containing both an amino group and a carboxyl group bound to a carbon which is designated the α-carbon.

The term “β-amino acid” refers to a molecule containing both an amino group and a carboxyl group in a β configuration. The abbreviation “b-” prior to an amino acid represents a beta configuration for the amino acid.

The term “naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | 3- | 1- | | Side-chain | |
|---|---|---|---|---|---|
| | Letter | Letter | Side-chain | charge | Hydropathy |
| Amino Acid | Code | Code | Polarity | (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive(10%) | −3.2 |
| | | | | neutral(90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acids” are glycine, alanine, proline, and analogs thereof. “Large hydrophobic amino acids” are valine, leucine, isoleucine, phenylalanine, methionine, tryptophan, tyrosine, and analogs thereof. “Polar amino acids” are serine, threonine, asparagine, glutamine, cysteine, and analogs thereof. “Charged amino acids” include positively charged amino acids and negatively charged amino acids. “Positively charged amino acids” include lysine, arginine, histidine, and analogs thereof. “Negatively charged amino acids” include aspartate, glutamate, and analogs thereof.

The term “amino acid analog” refers to a molecule which is structurally similar to an amino acid and which can be substituted for an amino acid in the formation of a peptidomimetic macrocycle. Amino acid analogs include, without limitation, β-amino acids and amino acids where the amino or carboxy group is substituted by a similarly reactive group (e.g., substitution of the primary amine with a secondary or tertiary amine, or substitution of the carboxy group with an ester).

The term “non-natural amino acid” refers to an amino acid which is not one of the twenty amino acids commonly found in peptides synthesized in nature, and known by the one letter abbreviations A, R, N, C, D, Q, E, G, H, I, L, K, M, F, P, S, T, W, Y and V. Non-natural amino acids or amino acid analogs include, without limitation, structures according to the following:

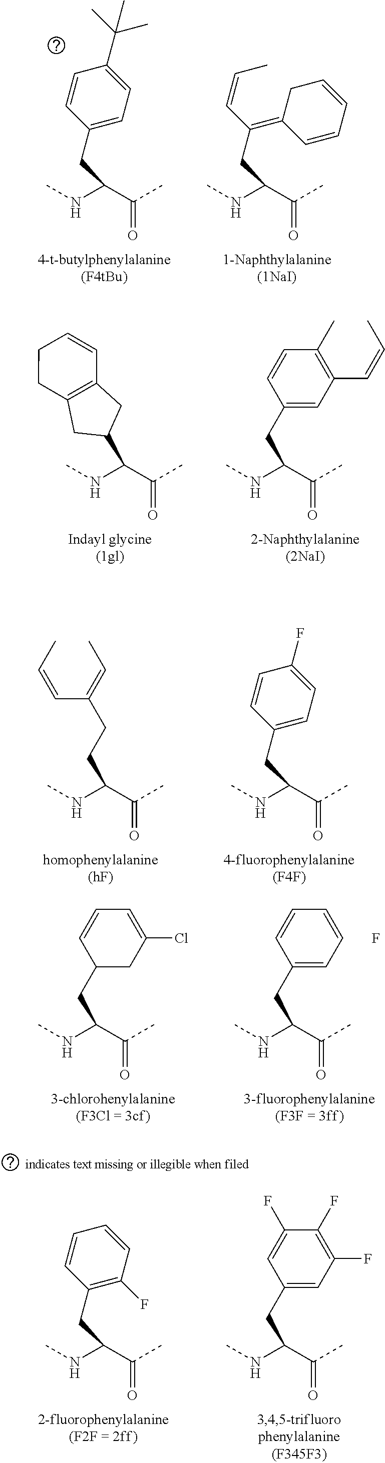

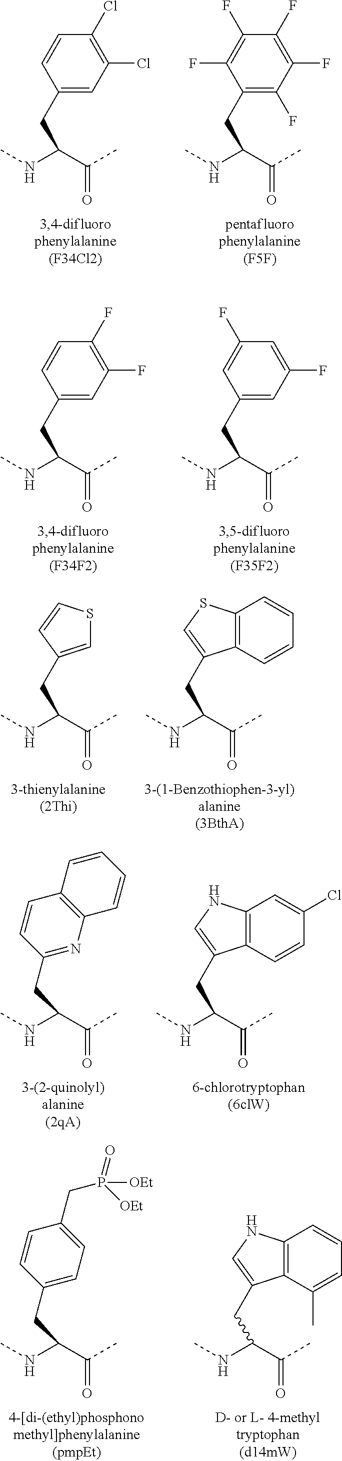

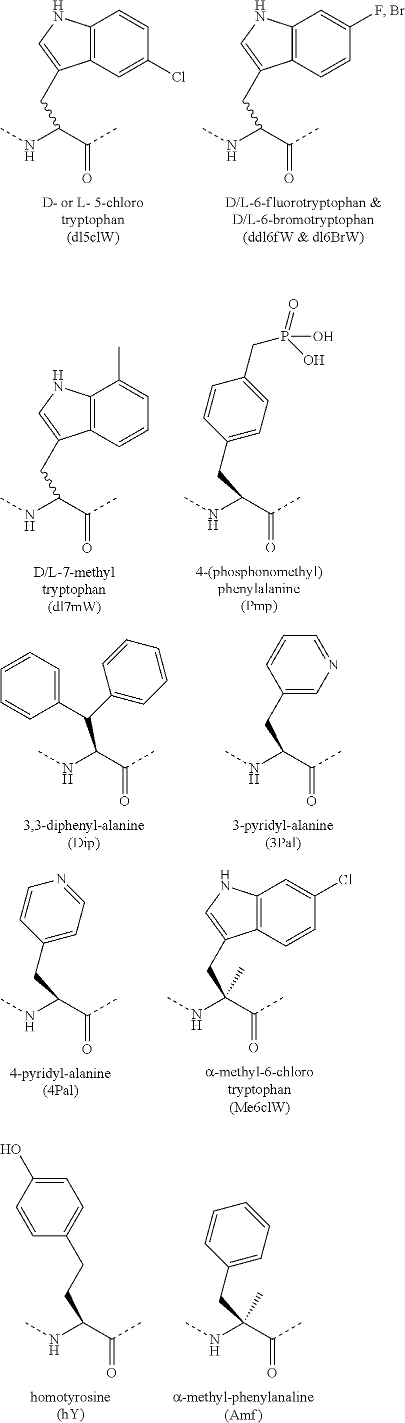

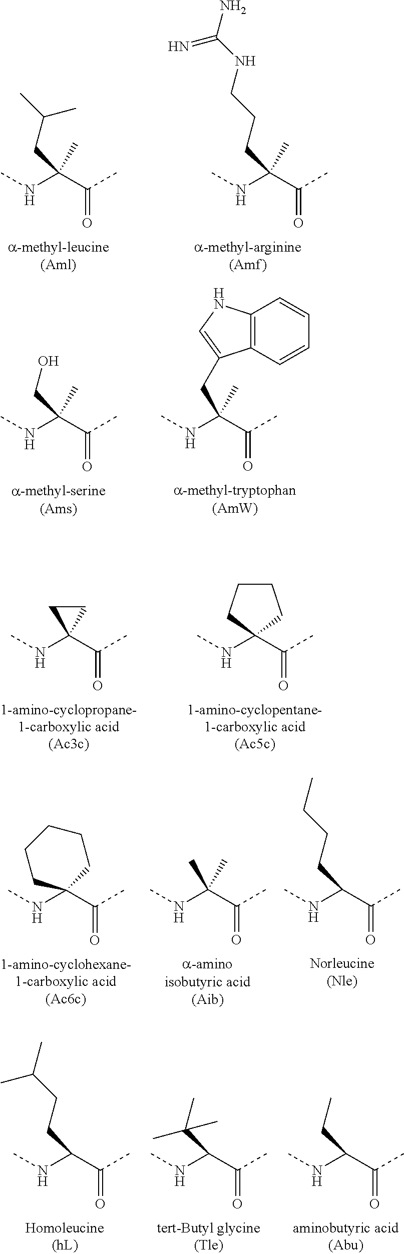

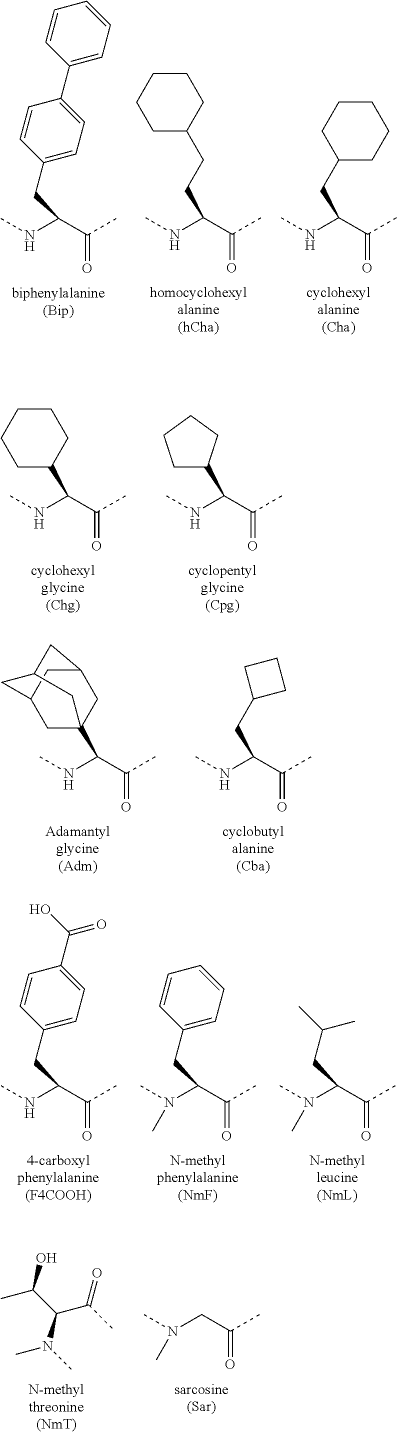

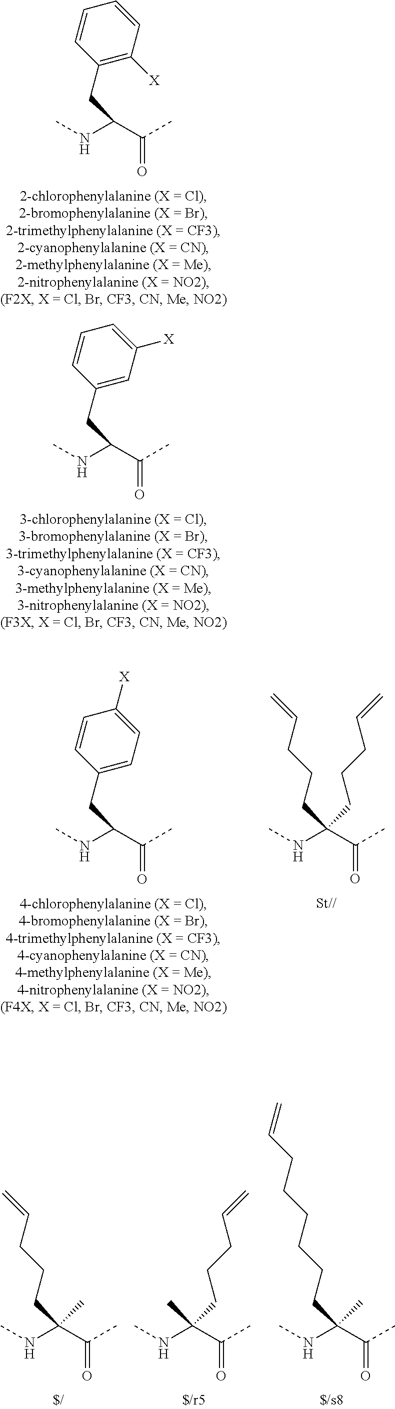

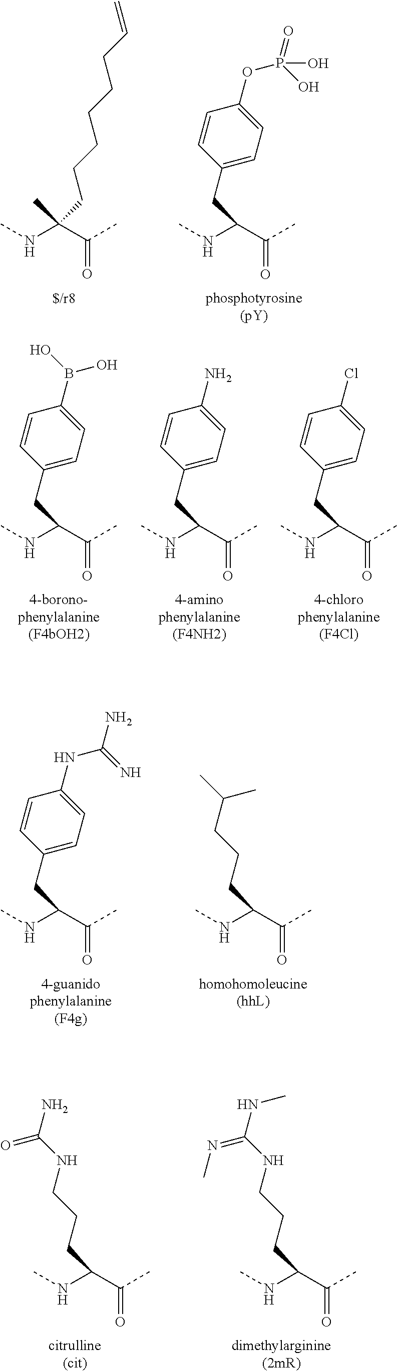

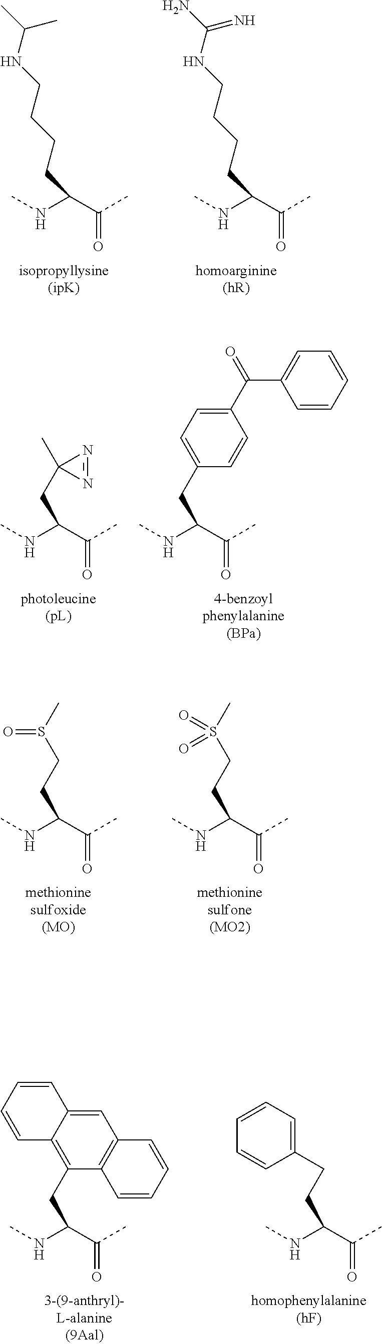

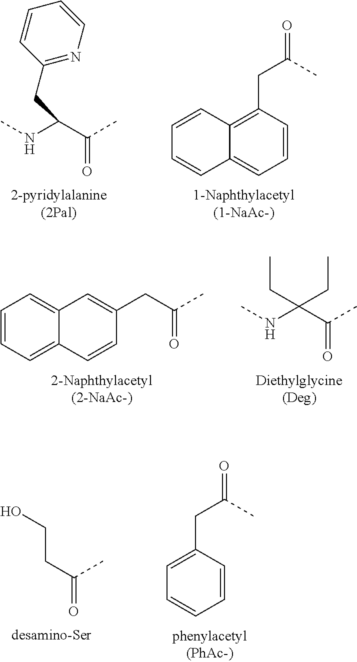

Amino acid analogs include β-amino acid analogs. Examples of β-amino acid analogs include, but are not limited to, the following: cyclic β-amino acid analogs; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl)-butyric acid; (S)-3-amino-4-(4-cyanophenyl)-butyric acid; (S)-3-amino-4-(4-fluorophenyl)-butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogs include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanine; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine.dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine.dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogs include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃)—OH; Nδ-benzyloxycarbonyl-L-ornithine; Nω-nitro-D-arginine; Nω-nitro-L-arginine; α-methyl-ornithine; 2,6-diaminoheptanedioic acid; L-ornithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-ornithine; (Nδ-4-methyltrityl)-D-ornithine; (Nδ-4-methyltrityl)-L-ornithine; D-ornithine; L-ornithine; Arg(Me)(Pbf)-OH; Arg(Me)₂-OH (asymmetrical); Arg(Me)2-OH (symmetrical); Lys(ivDde)-OH; Lys(Me)2-OH.HCl; Lys(Me3)-OH chloride; Nω-nitro-D-arginine; and Nω-nitro-L-arginine.

Amino acid analogs include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid γ,γ-di-t-butyl ester; Glu(OAll)-OH; L-Asu(OtBu)-OH; and pyroglutamic acid.

Amino acid analogs include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Amino acid analogs include analogs of phenylalanine and tyrosine. Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogs include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

Amino acid analogs include analogs of serine and threonine. Examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogs include analogs of tryptophan. Examples of amino acid analogs of tryptophan include, but are not limited to, the following: α-methyl-tryptophan; β-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some embodiments, amino acid analogs are racemic. In some embodiments, the D isomer of the amino acid analog is used. In some embodiments, the L isomer of the amino acid analog is used. In other embodiments, the amino acid analog comprises chiral centers that are in the R or S configuration. In still other embodiments, the amino group(s) of a β-amino acid analog is substituted with a protecting group, e.g., tert-butyloxycarbonyl (BOC group), 9-fluorenylmethyloxycarbonyl (FMOC), tosyl, and the like. In yet other embodiments, the carboxylic acid functional group of a β-amino acid analog is protected, e.g., as its ester derivative. In some embodiments the salt of the amino acid analog is used.

A “non-essential” amino acid residue is a residue that can be altered from the wild-type sequence of a polypeptide without abolishing or substantially abolishing its essential biological or biochemical activity (e.g., receptor binding or activation). An “essential” amino acid residue is a residue that, when altered from the wild-type sequence of the polypeptide, results in abolishing or substantially abolishing the polypeptide's essential biological or biochemical activity.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, e.g., is replaced with another amino acid residue from the same side chain family. Other examples of acceptable substitutions are substitutions based on isosteric considerations (e.g., norleucine for methionine) or other properties (e.g., 2-thienylalanine for phenylalanine).

The term “capping group” refers to the chemical moiety occurring at either the carboxy or amino terminus of the polypeptide chain of the subject peptidomimetic macrocycle. The capping group of a carboxy terminus includes an unmodified carboxylic acid (i.e. —COOH) or a carboxylic acid with a substituent. For example, the carboxy terminus can be substituted with an amino group to yield a carboxamide at the C-terminus. Various substituents include but are not limited to primary, secondary, and tertiary amines, including pegylated secondary amines. Representative secondary amine capping groups for the C-terminus include:

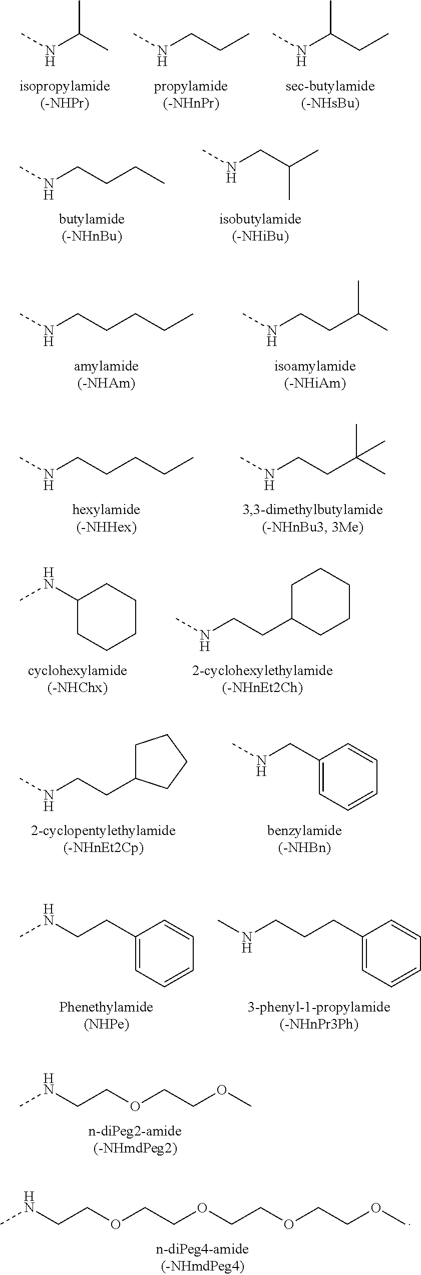

The capping group of an amino terminus includes an unmodified amine (i.e. —NH₂) or an amine with a substituent. For example, the amino terminus can be substituted with an acyl group to yield a carboxamide at the N-terminus. Various substituents include but are not limited to substituted acyl groups, including C₁-C₆ carbonyls, C₇-C₃₀ carbonyls, and pegylated carbamates. Representative capping groups for the N-terminus include:

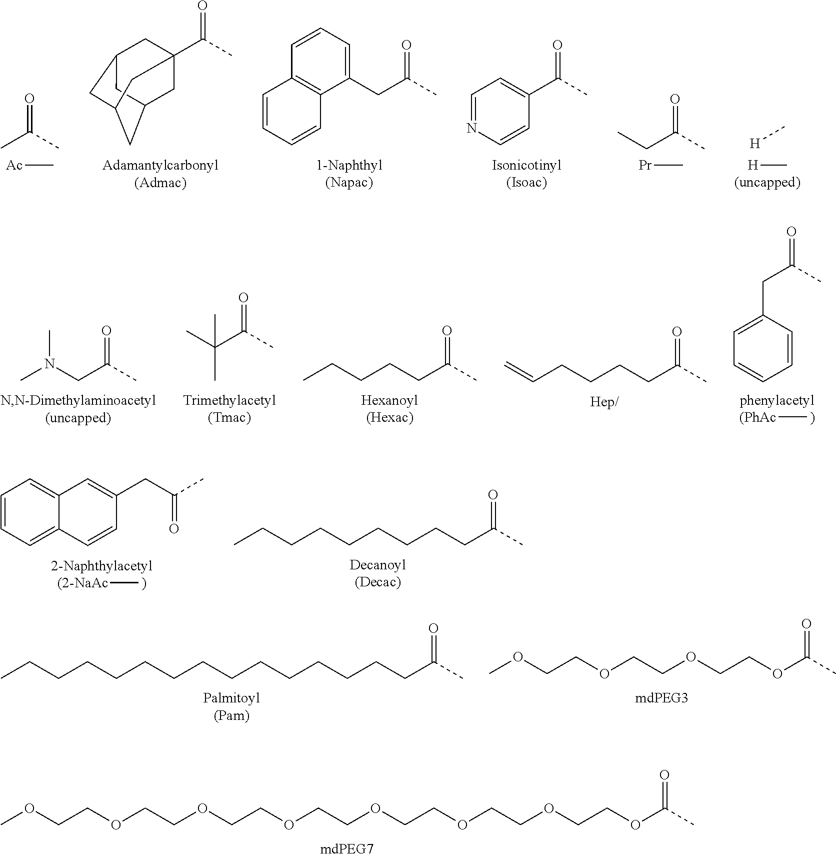

The term “member” as used herein in conjunction with macrocycles or macrocycle-forming linkers refers to the atoms that form or can form the macrocycle, and excludes substituent or side chain atoms. By analogy, cyclodecane, 1,2-difluoro-decane and 1,3-dimethyl cyclodecane are all considered ten-membered macrocycles as the hydrogen (—H) or fluoro substituents or methyl side chains do not participate in forming the macrocycle.

The symbol “

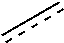

” when used as part of a molecular structure refers to a single bond or a trans or cis double bond.

The term “amino acid side chain” refers to a moiety attached to the α-carbon (or another backbone atom) in an amino acid. For example, the amino acid side chain for alanine is methyl, the amino acid side chain for phenylalanine is phenylmethyl, the amino acid side chain for cysteine is thiomethyl, the amino acid side chain for aspartate is carboxymethyl, the amino acid side chain for tyrosine is 4-hydroxyphenylmethyl, etc. Other non-naturally occurring amino acid side chains are also included, e.g., those that occur in nature (e.g., an amino acid metabolite) or those that are made synthetically (e.g., an α,α di-substituted amino acid).

The term “α,α di-substituted amino” acid refers to a molecule or moiety containing both an amino group and a carboxyl group bound to a carbon (the α-carbon) that is attached to two natural or non-natural amino acid side chains.

The term “polypeptide” encompasses two or more naturally or non-naturally-occurring amino acids joined by a covalent bond (e.g., an amide bond). Polypeptides as described herein include full length proteins (e.g., fully processed proteins) as well as shorter amino acid sequences (e.g., fragments of naturally-occurring proteins or synthetic polypeptide fragments).

The term “macrocyclization reagent” or “macrocycle-forming reagent” as used herein refers to any reagent which may be used to prepare a peptidomimetic macrocycle provided herein by mediating the reaction between two reactive groups. Reactive groups may be, e.g., an azide and alkyne, in which case macrocyclization reagents include, without limitation, Cu reagents such as reagents which provide a reactive Cu(I) species, such as CuBr, CuI or CuOTf, as well as Cu(II) salts such as Cu(CO₂CH₃)₂, CuSO₄, and CuCl₂ that can be converted in situ to an active Cu(I) reagent by the addition of a reducing agent such as ascorbic acid or sodium ascorbate. Macrocyclization reagents may additionally include, e.g., Ru reagents known in the art such as Cp*RuCl(PPh₃)₂, [Cp*RuCl]₄ or other Ru reagents which may provide a reactive Ru(II) species. In other cases, the reactive groups are terminal olefins. In such embodiments, the macrocyclization reagents or macrocycle-forming reagents are metathesis catalysts including, but not limited to, stabilized, late transition metal carbene complex catalysts such as Group VIII transition metal carbene catalysts. For example, such catalysts are Ru and Os metal centers having a +2 oxidation state, an electron count of 16 and pentacoordinated. In other examples, catalysts have W or Mo centers. Various catalysts are disclosed in Grubbs et al., Acc. Chem. Res. 1995, 28, 446-452, and U.S. Pat. No. 5,811,515; U.S. Pat. No. 7,932,397; U.S. Application No. 2011/0065915; U.S. Application No. 2011/0245477; Yu et al., Nature 2011, 479, 88; and Peryshkov et al., J. Am. Chem. Soc. 2011, 133, 20754. In yet other cases, the reactive groups are thiol groups. In such embodiments, the macrocyclization reagent is, e.g., a linker functionalized with two thiol-reactive groups such as halogen groups.

The term “halo” or “halogen” refers to fluorine, chlorine, bromine or iodine or a radical thereof.

The term “alkyl” refers to a hydrocarbon chain that is a straight chain or branched chain, containing the indicated number of carbon atoms. For example, C₁-C₁₀ indicates that the group has from 1-10 (inclusive) carbon atoms in it. In the absence of any numerical designation, “alkyl” is a chain (straight or branched) having 1-20 (inclusive) carbon atoms in it.

The term “alkylene” refers to a divalent alkyl (i.e. —R—).

The term “alkenyl” refers to a hydrocarbon chain that is a straight chain or branched chain having one or more carbon-carbon double bonds. The alkenyl moiety contains the indicated number of carbon atoms. For example, C₂-C₁₀ indicates that the group has from 2-10 (inclusive) carbon atoms in it. The term “lower alkenyl” refers to a C₂-C₆ alkenyl chain. In the absence of any numerical designation, “alkenyl” is a chain (straight or branched) having 2-20 (inclusive) carbon atoms in it.

The term “alkynyl” refers to a hydrocarbon chain that is a straight chain or branched chain having one or more carbon-carbon triple bonds. The alkynyl moiety contains the indicated number of carbon atoms. For example, C₂-C₁₀ indicates that the group has from 2-10 (inclusive) carbon atoms in it. The term “lower alkynyl” refers to a C₂-C₆ alkynyl chain. In the absence of any numerical designation, “alkynyl” is a chain (straight or branched) having 2-20 (inclusive) carbon atoms in it.

The term “aryl” refers to a 6-carbon monocyclic or 10-carbon bicyclic aromatic ring system wherein 0, 1, 2, 3, or 4 atoms of each ring are substituted by a substituent. Examples of aryl groups include phenyl, naphthyl and the like. The term “arylalkoxy” refers to an alkoxy substituted with aryl.

“Arylalkyl” refers to an aryl group, as defined above, wherein one of the aryl group's hydrogen atoms has been replaced with a C₁-C₅ alkyl group, as defined above. Representative examples of an arylalkyl group include, but are not limited to, 2-methylphenyl, 3-methylphenyl, 4-methylphenyl, 2-ethylphenyl, 3-ethylphenyl, 4-ethylphenyl, 2-propylphenyl, 3-propylphenyl, 4-propylphenyl, 2-butylphenyl, 3-butylphenyl, 4-butylphenyl, 2-pentylphenyl, 3-pentylphenyl, 4-pentylphenyl, 2-isopropylphenyl, 3-isopropylphenyl, 4-isopropylphenyl, 2-isobutylphenyl, 3-isobutylphenyl, 4-isobutylphenyl, 2-sec-butylphenyl, 3-sec-butylphenyl, 4-sec-butylphenyl, 2-t-butylphenyl, 3-t-butylphenyl and 4-t-butylphenyl.

“Arylamido” refers to an aryl group, as defined above, wherein one of the aryl group's hydrogen atoms has been replaced with one or more —C(O)NH₂ groups. Representative examples of an arylamido group include 2-C(O)NH2-phenyl, 3-C(O)NH₂-phenyl, 4-C(O)NH₂-phenyl, 2-C(O)NH₂-pyridyl, 3-C(O)NH₂-pyridyl, and 4-C(O)NH₂-pyridyl,

“Alkylheterocycle” refers to a C₁-C₅ alkyl group, as defined above, wherein one of the C₁-C₅ alkyl group's hydrogen atoms has been replaced with a heterocycle. Representative examples of an alkylheterocycle group include, but are not limited to, —CH₂CH₂-morpholine, —CH₂CH₂-piperidine, —CH₂CH₂CH₂-morpholine, and —CH₂CH₂CH₂-imidazole.

“Alkylamido” refers to a C₁-C₅ alkyl group, as defined above, wherein one of the C₁-C₅ alkyl group's hydrogen atoms has been replaced with a —C(O)NH₂ group. Representative examples of an alkylamido group include, but are not limited to, —CH₂—C(O)NH₂, —CH₂CH₂—C(O)NH₂, —CH₂CH₂CH₂C(O)NH₂, —CH₂CH₂CH₂CH₂C(O)NH₂, —CH₂CH₂CH₂CH₂CH₂C(O)NH₂, —CH₂CH(C(O)NH₂)CH₃, —CH₂CH(C(O)NH₂)CH₂CH₃, —CH(C(O)NH₂)CH₂CH₃, —C(CH₃)2CH₂C(O)NH₂, —CH₂—CH₂—NH—C(O)—CH₃, —CH₂—CH₂—NH—C(O)—CH₃—CH3, and —CH₂—CH₂—NH—C(O)—CH═CH₂.

“Alkanol” refers to a C₁-C₅ alkyl group, as defined above, wherein one of the C₁-C₅ alkyl group's hydrogen atoms has been replaced with a hydroxyl group. Representative examples of an alkanol group include, but are not limited to, —CH₂OH, —CH₂CH₂OH, —CH₂CH₂CH₂OH, —CH₂CH₂CH₂CH₂OH, —CH₂CH₂CH₂ CH₂CH₂OH, —CH₂CH(OH)CH₃, —CH₂CH(OH)CH₂CH₃, —CH(OH)CH₃ and —C(CH₃)₂CH₂OH.

“Alkylcarboxy” refers to a C₁-C₅ alkyl group, as defined above, wherein one of the C₁-C₅ alkyl group's hydrogen atoms has been replaced with a —COOH group. Representative examples of an alkylcarboxy group include, but are not limited to, —CH₂COOH, —CH₂CH₂COOH, —CH₂CH₂CH₂COOH, —CH₂CH₂CH₂CH₂COOH, —CH₂CH(COOH)CH₃, —CH₂CH₂CH₂CH₂CH₂COOH, —CH₂CH(COOH)CH₂CH₃, —CH(COOH)CH₂CH₃ and —C(CH₃)2CH₂COOH.

The term “cycloalkyl” as employed herein includes saturated and partially unsaturated cyclic hydrocarbon groups having 3-12 carbons, preferably 3-8 carbons, and more preferably 3-6 carbons, wherein the cycloalkyl group additionally is optionally substituted. Some cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2, 3, or 4 atoms of each ring are substituted by a substituent. Examples of heteroaryl groups include pyridyl, furyl or furanyl, imidazolyl, benzimidazolyl, pyrimidinyl, thiophenyl or thienyl, quinolinyl, indolyl, thiazolyl, and the like.

The term “heteroarylalkyl” or the term “heteroaralkyl” refers to an alkyl substituted with a heteroaryl. The term “heteroarylalkoxy” refers to an alkoxy substituted with heteroaryl.

The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of O, N, or S if monocyclic, bicyclic, or tricyclic, respectively), wherein 0, 1, 2 or 3 atoms of each ring are substituted by a substituent. Examples of heterocyclyl groups include piperazinyl, pyrrolidinyl, dioxanyl, morpholinyl, tetrahydrofuranyl, and the like.

The term “substituent” refers to a group replacing a second atom or group such as a hydrogen atom on any molecule, compound or moiety. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, and cyano groups.

In some embodiments, the compounds of this invention contain one or more asymmetric centers and thus occur as racemates and racemic mixtures, single enantiomers, individual diastereomers and diastereomeric mixtures. All such isomeric forms of these compounds are included in the present invention unless expressly provided otherwise. In some embodiments, the compounds of this invention are also represented in multiple tautomeric forms, in such instances, the invention includes all tautomeric forms of the compounds described herein (e.g., if alkylation of a ring system results in alkylation at multiple sites, the invention includes all such reaction products). All such isomeric forms of such compounds are included in the present invention unless expressly provided otherwise. All crystal forms of the compounds described herein are included in the present invention unless expressly provided otherwise.

As used herein, the terms “increase” and “decrease” mean, respectively, to cause a statistically significantly (i.e. p<0.1) increase or decrease of at least 5%.

As used herein, the recitation of a numerical range for a variable is intended to convey that the variable is equal to any of the values within that range. Thus, for a variable which is inherently discrete, the variable is equal to any integer value within the numerical range, including the end-points of the range. Similarly, for a variable which is inherently continuous, the variable is equal to any real value within the numerical range, including the end-points of the range. As an example, and without limitation, a variable which is described as having values between 0 and 2 takes the values 0, 1 or 2 if the variable is inherently discrete, and takes the values 0.0, 0.1, 0.01, 0.001, or any other real values ≧0 and ≦2 if the variable is inherently continuous.

As used herein, unless specifically indicated otherwise, the word “or” is used in the inclusive sense of “and/or” and not the exclusive sense of “either/or.”

The term “on average” represents the mean value derived from performing at least three independent replicates for each data point.

The term “biological activity” encompasses structural and functional properties of a macrocycle of the invention. Biological activity is, e.g., structural stability, alpha-helicity, affinity for a target, resistance to proteolytic degradation, cell penetrability, intracellular stability, in vivo stability, or any combination thereof.

### Peptidomimetic Macrocycles of the Invention

PTH is a polypeptide consisting of 84 amino acids and its main target organs are bone, cartilage and kidney. It is known that after binding to the receptor of a target cell, PTH initiates various intra- and inter-cellular cascades including the promotion of the production of intracellular cyclic adenosine monophosphate (cAMP), the phosphorylation of intracellular proteins, the flow of calcium into a cell, the stimulation of the metabolic path of membrane phospholipids, the activation of intracellular enzyme and the secretion of lysosome enzyme. Expression of PTH gene is subjected to suppressive control mainly with activated vitamin D₃. Abnormal production of PTH in vivo causes various diseases. Examples of the diseases are hypoparathyroidism, primary hyperparathyroidism and secondary hyperparathyroidism associated with an increase of PTH production. Chronic, excessive production of PTH is known as hyperparathyroidism (HPT). Overproduction of parathyroid hormone leads to an elevated blood calcium level and decreased blood phosphate level. Calcium is removed from bones and calcium absorption from the gastrointestinal (GI) tract increases. The kidneys attempt to compensate for the increased blood calcium level by secreting excess calcium in the urine, which can result in the formation of kidney stones. The effects of increased PTH levels are seen not only in the kidneys, but also in the skeleton, stomach and intestines, the nervous system, and the muscles.

PTH has an anabolic effect on bone that involves a domain for protein kinase C activation (amino acid residues 28-34) as well as a domain for adenylate cyclase activation (amino acid residues 1-7). Various catabolic forms of clipped or fragmented PTH peptides also are found in circulation, most likely formed by intraglandular or peripheral metabolism. For example, whole PTH can be cleaved between amino acids 34 and 35 to produce a (1-34) PTH N-terminal fragment and a (35-84) PTH C-terminal fragment. Likewise, clipping can occur between either amino acids 36 and 37 or 37 and 38.

Primary hyperparathyroidism is a systemic disease caused by the excessive PTH secretion from one or more parathyroid glands and about 90% of the patients are affected by parathyroid tumor. The secondary hyperparathyroidism is a disease developed by the excessive secretion of PTH caused by the metabolic disturbance of activated vitamin D, calcium and phosphorus of a patient of chronic renal failure resulting in the growth of parathyroid gland to exhibit resistance to 1α,25-dihydroxyvitamin D₃ of physiological concentration and further progress hyperplacia. There are many cases accompanying ostealgia and arthralgia owing to the increase of bone resorption by excessive PTH. Further, the disease sometimes develops symptoms other than bone part such as ectopic calcification of soft tissue and arterial wall caused by hypercalcemia and hyperphosphatemia.

Reported PTH modulators such as Sensipar (Cinacalcel), only addresses 30-40% of potential patients and has considerable GI side effects. Thus, provided herein are effective PTH antagonists that minimize side effects. Additionally, reported PTH modulators, such as calcimimetic (AMG-416, aka KAI-4169, Phase 2), are delivered intravenously and thus cannot address non-dialysis SHPT or PHPT because intravenous delivery cannot be used to treat hypercalcemia of malignancy (HOM).

Therefore, there remains a need for agents with PTH activity (e.g., agonist and antagonist activity, including partial agonist or antagonist activity) which have enhanced half-life, reduced side-effect profile, and are convenient to administer.

The present invention provides pharmaceutical formulations comprising an effective amount of peptidomimetic macrocycles or pharmaceutically acceptable salts thereof. The term “peptidomimetic macrocycle” is meant to include pharmaceutically acceptable salts thereof unless otherwise conveyed. The peptidomimetic macrocycles provided herein are cross-linked (e.g., stapled or stitched) and possess improved pharmaceutical properties relative to their corresponding uncross-linked peptidomimetic macrocycles. These improved properties include improved bioavailability, enhanced chemical and in vivo stability, increased potency, and reduced immunogenicity (i.e. fewer or less severe injection site reactions).

The sequence of human PTH (1-34) is SVSEIQLMHNLGKHLNSMERVEWLRKKLQDVHNF (SEQ ID NO: 1). The sequence of human PTH (3-34) is SEIQLMHNLGKHLNSMERVEWLRKKLQDVHNF (SEQ ID NO: 2). The sequence of human PTH (7-34) is LMHNLGKHLNSMERVEWLRKKLQDVHNF (SEQ ID NO: 3). The sequence of human PTHrP (1-36) is AVSEHQLLHDKGKSIQDLRRRFFLHHLIAEIHTAEY (SEQ ID NO: 4). The sequence of human PTHrP (7-36) is LLHDKGKSIQDLRRRFFLHHLIAEIHTAEY (SEQ ID NO: 5).

In some embodiments, the peptide sequence of a peptidomimetic macrocycle is derived from a parathyroid hormone (PTH) peptide. For example, the peptide sequences are derived from human PTH (1-34), human PTH (3-34) or human PTH (7-34).

In some embodiments, the peptidomimetic macrocycle peptide sequences are derived from a PTH peptide and/or a parathyroid hormone-related peptide (PTHrP). For example, the peptidomimetic macrocycle peptide sequences are derived from human PTHrP (1-36) or human PTHrP (7-36) or human PTHrP (7-34).

In some embodiments, the peptidomimetic macrocycle peptide sequences are derived from a PTH peptide and a PTHrP peptide. For example, the peptidomimetic macrocycle peptide sequences are derived from human PTH (1-34) and human PTHrP (1-36). For example, the peptidomimetic macrocycle peptide sequences are derived from human PTH (1-34) and human PTHrP (7-36). For example, the peptidomimetic macrocycle peptide sequences are derived from human PTH (3-34) and human PTHrP (1-36). For example, the peptidomimetic macrocycle peptide sequences are derived from human PTH (3-34) and human PTHrP (7-36). For example, the peptidomimetic macrocycle peptide sequences are derived from human PTH (7-34) and human PTHrP (1-36). For example, the peptidomimetic macrocycle peptide sequences are derived from human PTH (7-34) and human PTHrP (7-36).

In some embodiments, a peptidomimetic macrocycle peptide sequence is derived from human PTH (7-14) and PTHrP (15-34). In other embodiments, a peptidomimetic macrocycle peptide sequence is derived from human PTHrP (7-21) and PTH (22-34). In other embodiments, a peptidomimetic macrocycle peptide sequence is derived from human PTH (7-14), human PTHrP (15-21) and PTH (22-34) or PTH (22-36). In other embodiments, a peptidomimetic macrocycle peptide sequence is derived from human PTH (7-18), human PTHrP (19-21) and PTH (22-34).

In some embodiments, a peptidomimetic macrocycle peptide derived from a human PTH peptide is a peptide comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 amino acids from a human PTH peptide sequence. In some embodiments, a peptidomimetic macrocycle peptide derived from a human PTHrP is a peptide comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids from a human PTHrP peptide sequence. In some embodiments, a peptidomimetic macrocycle peptide derived from a human PTH peptide and a human PTHrP peptide is a peptide comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 amino acids from a human PTH sequence and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids from a human PTHrP peptide sequence.

In some embodiments, a peptidomimetic macrocycle peptide derived from a human PTH peptide and/or a human PTHrP sequence is a peptide comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids that are different from the selected sequences from which the peptide is derived. In some embodiments, a peptidomimetic macrocycle peptide derived from a human PTH peptide and/or a human PTHrP sequence is a peptide comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 mutations. In some embodiments, a peptidomimetic macrocycle peptide derived from a human PTH peptide and/or a human PTHrP sequence is a peptide comprising a mutation at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36. In some embodiments, mutations are mutations of non-essential amino acids. In some embodiments, mutations are mutations of essential amino acids. In some embodiments, mutations are mutations of hydrophobic amino acids. In some embodiments, mutations are mutations of naturally occurring amino acids. In some embodiments, mutations are mutations to a conservative amino acid. In some embodiments, a peptidomimetic macrocycle peptide derived from a human PTH peptide and/or a human PTHrP sequence is a peptide comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acid analogues. In some embodiments, a peptidomimetic macrocycle peptide derived from a human PTH peptide and/or a human PTHrP sequence can be a peptide comprising 1 or 2 capping groups.

A non-limiting list of suitable PTH, PTHrP, and PTH and PTHrP derived peptidomimetic macrocycles for use in the present invention are given in Tables 1a and 1b below. A non-limiting list of suitable PTH, PTHrP, and PTH and PTHrP derived linear peptidomimetics for use in the present invention is given in Tables 2a and 2b. In the tables shown herein, some peptides possess a free amino terminus (shown as H—) and some peptides possess a carboxamide terminus (shown as —NH2). A non-limiting list of suitable PTH, PTHrP, and PTH and PTHrP derived peptidomimetic macrocycles for use in the present invention are given in Tables 3a, 3b, 5, 6, and 7 below. A non-limiting list of suitable amino acid mutations for use in the present invention is given in Table 4. Table 8 shows exemplary peptidomimetic macrocycles.

TABLE 1
Peptidomimetic Macrocycles

| SEQ | | | | | | | | | | | | | | | | | | | | |
|---|---|---|---|---|---|---|---|---|---|---|---|---|---|---|---|---|---|---|---|---|
| ID | SP# | 0 | 1 | 2 | 3 | 4 | 5 | 6 | 7 | 8 | 9 | 10 | 11 | 12 | 13 | 14 | 15 | 16 | 17 | 18 |
| 6 | PTH | H- | S | V | S | E | I | Q | L | M | H | N | L | G | K | H | L | N | S | M |
| 7 | PTHrP | H- | A | V | S | E | H | Q | L | L | H | D | K | G | K | S | I | Q | D | L |

| SEQ | | | | | | | | | | | | | | | | | | | |
|---|---|---|---|---|---|---|---|---|---|---|---|---|---|---|---|---|---|---|---|
| ID | 19 | 20 | 21 | 22 | 23 | 24 | 25 | 26 | 27 | 28 | 29 | 30 | 31 | 32 | 33 | 34 | 35 | 36 | 37 |
| 6 | E | R | V | E | W | L | R | K | K | L | Q | D | V | H | N | F | | | -OH |
| 7 | R | R | R | F | F | L | H | H | L | I | A | E | I | H | T | A | E | Y | -NH₂ |

Table 1a
Peptidomimetic Macrocycles

| SEQ | | | | | | | | | | | | | | | | | | | | |
|---|---|---|---|---|---|---|---|---|---|---|---|---|---|---|---|---|---|---|---|---|
| ID | SP# | 0 | 1 | 2 | 3 | 4 | 5 | 6 | 7 | 8 | 9 | 10 | 11 | 12 | 13 | 14 | 15 | 16 | 17 | 18 |
| 8 | 2 | H- | | | | | | | L | L | H | N | L | G | K | H | L | N | S | L |
| 9 | 3 | H- | | | | | | | L | L | H | N | L | G | K | H | L | N | S | L |
| 10 | 4 | H- | | | | | | | L | L | H | N | L | G | K | H | L | N | S | L |
| 11 | 5 | H- | | | | | | | L | L | H | N | L | G | K | H | L | N | S | L |
| 12 | 6 | H- | | | | | | | L | L | H | N | L | G | K | H | L | N | S | L |
| 13 | 7 | H- | | | | | | | L | L | H | N | L | G | K | H | L | N | S | L |
| 14 | 8 | H- | | | | | | | L | L | H | N | L | G | K | H | L | N | S | L |
| 15 | 9 | H- | | | | | | | L | L | H | N | L | G | K | H | L | N | S | L |
| 16 | 10 | H- | | | | | | | L | L | H | N | L | G | K | H | L | N | S | L |
| 17 | 11 | H- | | | | | | | L | L | H | N | L | G | K | H | L | N | S | L |
| 18 | 12 | H- | | | | | | | L | L | H | N | L | G | K | H | $ | N | S | L |
| 19 | 13 | H- | | | | | | | L | L | H | N | L | G | K | $ | L | N | S | $ |
| 20 | 14 | H- | | | | | | | L | L | H | N | L | G | $ | H | L | N | $ | L |
| 21 | 15 | H- | | | | | | | L | L | H | N | L | $ | K | H | L | $ | S | L |
| 22 | 16 | H- | | | | | | | L | L | H | N | $ | G | K | H | $ | N | S | L |
| 23 | 17 | H- | | | | | | | L | L | H | $ | L | G | K | $ | L | N | S | L |
| 24 | 18 | H- | | | | | | | L | L | $ | N | L | G | $ | H | L | N | S | L |
| 25 | 19 | H- | | | | | | | L | $ | H | N | L | $ | K | H | L | N | S | L |
| 26 | 20 | H- | | | | | | | $ | L | H | N | $ | G | K | H | L | N | S | L |
| 27 | 21 | H- | S | V | S | E | I | $ | L | L | H | $ | L | G | K | H | L | N | S | L |
| 28 | 22 | H- | S | V | S | E | $ | Q | L | L | $ | N | L | G | K | H | L | N | S | L |
| 29 | 23 | H- | S | V | S | $ | I | Q | L | $ | H | N | L | G | K | H | L | N | S | L |
| 30 | 24 | H- | S | V | $ | E | I | Q | $ | L | H | N | L | G | K | H | L | N | S | L |
| 31 | 25 | H- | S | $ | S | E | I | $ | L | L | H | N | L | G | K | H | L | N | S | L |
| 32 | 26 | H- | $ | V | S | E | $ | Q | L | L | H | N | L | G | K | H | L | N | S | L |
| 33 | 27 | HEP- | S | V | S | $ | I | Q | L | L | H | N | L | G | K | H | L | N | S | L |
| 34 | 28 | H- | | | | | | | L | L | H | N | L | G | K | H | L | N | S | L |
| 35 | 29 | H- | | | | | | | L | L | H | N | L | G | K | H | L | N | S | L |
| 36 | 30 | H- | | | | | | | L | L | H | N | L | G | K | H | L | N | S | L |
| 37 | 31 | H- | | | | | | | L | L | H | N | L | G | K | H | L | N | S | L |
| 38 | 32 | H- | | | | | | | L | L | H | N | L | G | K | H | L | N | S | L |
| 39 | 33 | H- | | | | | | | L | L | H | N | L | G | K | H | L | N | S | L |
| 40 | 34 | H- | | | | | | | L | L | H | N | L | G | K | H | L | N | S | L |
| 41 | 35 | H- | | | | | | | L | L | H | N | L | G | K | H | L | N | S | L |
| 42 | 36 | H- | | | | | | | L | L | H | N | L | G | K | H | L | N | S | $r8 |
| 43 | 37 | H- | | | | | | | L | L | H | N | L | G | K | H | L | N | S | $r8 |
| 44 | 38 | H- | | | | | | | L | L | H | N | L | G | K | H | L | N | $r8 | L |
| 45 | 39 | H- | | | | | | | L | L | H | N | L | G | K | H | L | N | $r8 | L |
| 46 | 40 | H- | | | | | | | L | L | H | N | L | G | K | H | $r8 | N | S | L |
| 47 | 41 | H- | | | | | | | L | L | H | N | L | G | K | H | $r8 | N | S | L |
| 48 | 42 | H- | | | | | | | L | L | H | N | L | G | K | $r8 | L | N | S | L |
| 49 | 43 | H- | | | | | | | L | L | H | N | L | G | K | $r8 | L | N | S | L |
| 50 | 44 | H- | | | | | | | L | L | H | N | L | $r8 | K | H | L | N | S | L |
| 51 | 45 | H- | | | | | | | L | L | H | N | $r8 | G | K | H | L | N | S | $ |
| 52 | 46 | H- | | | | | | | L | L | H | $r8 | L | G | K | H | L | N | $ | L |
| 53 | 47 | H- | | | | | | | L | L | $r8 | N | L | G | K | H | L | $ | S | L |
| 54 | 48 | H- | | | | | | | L | $r8 | H | N | L | G | K | H | $ | N | S | L |
| 55 | 49 | H- | | | | | | | L | $r8 | H | N | L | G | K | H | $ | N | S | L |
| 56 | 50 | H- | | | | | | | $r8 | L | H | N | L | G | K | $ | L | N | S | L |
| 57 | 51 | H- | S | V | S | E | I | $r8 | L | L | H | N | L | G | $ | H | L | N | S | L |
| 58 | 52 | H- | S | V | S | E | $r8 | Q | L | L | H | N | L | $ | K | H | L | N | S | L |
| 59 | 53 | H- | S | V | S | $r8 | I | Q | L | L | H | N | $ | G | K | H | L | N | S | L |
| 60 | 54 | H- | S | V | $r8 | E | I | Q | L | L | H | $ | L | G | K | H | L | N | S | L |
| 61 | 55 | H- | S | $r8 | S | E | I | Q | L | L | $ | N | L | G | K | H | L | N | S | L |
| 62 | 56 | H- | $r8 | V | S | E | I | Q | L | $ | H | N | L | G | K | H | L | N | S | L |
| 63 | 57 | H- | | | | | | | L | L | H | N | L | G | $ | H | L | N | $ | L |
| 64 | 58 | H- | | | | | | | L | L | H | N | L | A | $ | H | L | N | $ | L |
| 65 | 59 | H- | | | | | | | L | L | H | N | L | A | K | $ | L | N | S | $ |
| 66 | 60 | H- | | | | | | | L | L | H | N | L | A | K | H | L | N | S | $ |
| 67 | 61 | H- | | | | | | | L | L | H | N | L | A | $ | H | L | N | $ | L |
| 68 | 62 | H- | | | | | | | L | L | H | N | L | A | K | $ | L | N | S | $ |
| 69 | 63 | H- | | | | | | | L | L | H | Q | hR | A | $ | W | I | Q | $ | L |
| 70 | 64 | H- | | | | | | | L | L | H | Q | hR | A | K | $ | I | Q | D | $ |
| 71 | 65 | H- | | | | | | | L | L | H | Q | hR | A | K | W | I | Q | D | $ |
| 72 | 66 | H- | | | | | | | L | L | H | Q | hR | A | $ | W | I | Q | $ | L |
| 73 | 67 | H- | | | | | | | L | L | H | Q | hR | A | K | $ | I | Q | D | $ |
| 74 | 68 | H- | | | | | | | L | L | H | Q | hR | A | K | $ | I | Q | D | $ |
| 75 | 69 | H- | | | | | | | L | L | H | Q | L | G | K | $ | I | Q | D | $ |
| 76 | 70 | H- | | | | | | | L | L | H | Q | L | G | K | $ | L | N | S | $ |
| 77 | 71 | H- | | | | | | | L | L | H | Q | K | G | K | $ | I | Q | D | $ |
| 78 | 72 | H- | | | | | | | L | L | H | Q | hR | A | K | $ | I | Q | D | $ |
| 79 | 73 | H- | | | | | | | L | L | H | Q | hR | A | K | $ | L | N | S | $ |
| 80 | 74 | H- | | | | | | | F | L | H | Q | hR | A | K | $ | L | N | S | $ |
| 81 | 75 | H- | | | | | | | F | L | H | Q | hR | w | K | $ | L | N | S | $ |
| 82 | 76 | H- | | | | | | | L | L | H | D | K | G | K | $ | I | Q | D | $ |
| 83 | 77 | H- | | | | | | | L | L | H | Q | hR | A | K | $ | I | Q | D | $ |
| 84 | 78 | H- | | | | | | | L | L | H | Q | hR | A | K | W | I | Q | D | L |
| 85 | 79 | H- | | | | | | | L | L | H | Q | hR | A | K | $ | I | Q | D | $ |
| 86 | 80 | H- | | | | | | | L | L | H | Q | hR | A | K | $ | I | Q | D | $ |
| 87 | 81 | H- | | | | | | | L | L | H | Q | hR | A | K | $ | I | Q | D | $ |
| 88 | 82 | H- | | | | | | | L | L | H | Q | hR | A | K | $ | I | Q | D | $ |
| 89 | 83 | H- | | | | | | | L | L | H | Q | hR | A | K | $ | I | Q | D | $ |
| 90 | 84 | H- | | | | | | | L | L | H | Q | hR | A | K | $ | I | Q | D | $ |
| 91 | 85 | H- | | | | | | | L | L | H | Q | hR | A | K | $ | I | Q | D | $ |
| 92 | 86 | H- | | | | | | | L | L | H | Q | hR | A | $ | W | I | Q | $ | L |
| 93 | 87 | H- | | | | | | | L | L | H | Q | $ | A | K | W | $ | Q | D | L |
| 94 | 88 | H- | | | | | | | L | L | H | $ | hR | A | K | $ | I | Q | D | L |
| 95 | 89 | H- | | | | | | | L | L | $ | Q | hR | A | $ | W | I | Q | D | L |
| 96 | 90 | H- | | | | | | | L | $ | H | Q | hR | $ | K | W | I | Q | D | L |
| 97 | 91 | H- | | | | | | | $ | L | H | Q | $ | A | K | W | I | Q | D | L |
| 98 | 92 | H- | | | | | | | L | L | H | $ | hR | A | K | W | I | Q | D | L |
| 99 | 93 | H- | | | | | | | L | L | H | Q | hR | $ | K | W | I | $ | D | L |
| 100 | 94 | H- | | | | | | | F | L | H | Q | hR | A | K | $ | I | Q | D | $ |
| 101 | 95 | H- | | | | | | | F4 | L | H | Q | hR | A | K | $ | I | Q | D | $ |
| | | | | | | | | | Cl | | | | | | | | | | | |
| 102 | 96 | H- | | | | | | | L | Nle | H | Q | hR | A | K | $ | I | Q | D | $ |
| 103 | 97 | H- | | | | | | | L | L | H | Q | hR | A | K | $ | I | Q | D | $ |
| 104 | 98 | H- | | | | | | | L | L | H | Q | hR | w | K | $ | I | Q | D | $ |
| 105 | 99 | H- | | | | | | | F | Nle | H | Q | hR | A | K | $ | I | Q | D | $ |
| 106 | 100 | H- | | | | | | | L | L | H | Q | hR | A | S | $ | I | Q | D | $ |
| 107 | 101 | H- | | | | | | | L | L | H | A | hR | A | K | $ | I | Q | D | $ |
| 108 | 102 | H- | | | | | | | L | L | H | D | hR | A | K | $ | I | Q | D | $ |
| 109 | 103 | H- | | | | | | | L | L | H | Q | hR | A | S | $ | I | Q | D | $ |
| 110 | 104 | H- | | | | | | | L | L | H | Q | hR | A | S | $ | I | Q | D | $ |
| 111 | 105 | H- | | | | | | | L | L | H | Q | hR | A | K | $ | I | Q | D | $ |
| 112 | 106 | H- | | | | | | | L | L | H | Q | hR | A | K | $ | I | Q | D | $ |
| 113 | 107 | H- | | | | | | | L | L | H | Q | hR | A | K | $ | I | Q | D | $ |
| 114 | 108 | H- | | | | | | | L | L | H | Q | hR | A | K | $ | I | Q | D | $ |
| 115 | 109 | H- | | | | | | | L | L | H | Q | hR | A | K | $ | I | Q | D | $ |
| 116 | 110 | H- | | | | | | | L | L | H | Q | hR | A | K | $ | I | Q | D | $ |
| 117 | 111 | Ph | S | V | Deg | E | H | Q | L | L | H | Q | hR | A | K | $ | I | Q | D | $ |
| | | Ac- | | | | | | | | | | | | | | | | | | |
| 118 | 112 | H- | | | | | | | L | L | H | N | L | G | K | H | L | N | S | $ |
| 119 | 113 | H- | | | | | | | L | L | H | N | L | G | K | H | L | N | S | $r5 |
| 120 | 114 | H- | | | | | | | L | L | H | Q | hR | A | K | W | I | Q | D | $ |
| 121 | 115 | H- | | | | | | | L | L | H | Q | hR | A | K | W | I | Q | D | $r5 |
| 122 | 116 | H- | | | | | | | L | L | H | Q | hR | A | K | W | I | Q | D | $ |
| 123 | 117 | H- | | | | | | | L | L | H | Q | hR | A | K | $ | I | Q | D | St |
| 124 | 118 | H- | | | | | | | L | L | H | Q | hR | A | K | $r5 | I | Q | D | $ |
| 125 | 119 | H- | | | | | | | L | L | H | Q | hR | A | K | W | Sr8 | Q | D | L |
| 126 | 120 | H- | | | | | | | L | L | H | Q | hR | A | K | $r8 | I | Q | D | L |
| 127 | 121 | H- | | | | | | | L | L | H | Q | hR | A | K | W | I | Q | $r8 | L |
| 128 | 122 | H- | | | | | | | L | L | H | N | $ | G | K | H | $ | N | S | L |
| 129 | 123 | H- | | | | | | | L | L | H | $ | L | G | K | $ | L | N | S | L |
| 130 | 124 | H- | | | | | | | L | L | H | N | L | G | $ | H | L | N | S | L |
| 131 | 125 | H- | | | | | | | L | L | H | N | $ | G | K | H | $ | N | S | L |
| 132 | 126 | H- | | | | | | | L | L | H | $ | L | G | K | $ | L | N | S | L |
| 133 | 127 | H- | | | | | | | L | L | H | N | $ | G | K | H | $ | N | S | L |
| 134 | 128 | H- | | | | | | | L | L | H | N | $ | G | K | H | $ | N | S | L |
| 135 | 129 | H- | | | | | | | L | L | H | $ | L | G | K | $ | L | N | S | L |
| 136 | 130 | H- | | | | | | | L | L | H | N | L | G | $ | $ | L | N | $ | L |
| 137 | 131 | H- | | | | | | | L | L | H | $ | L | G | K | H | L | N | S | L |
| 138 | 132 | H- | S | V | S | E | I | Q | L | L | H | N | L | G | $ | $ | L | N | $ | L |
| 139 | 133 | H- | S | V | S | E | I | Q | L | L | H | N | L | A | $ | H | L | N | $ | L |
| 140 | 134 | H- | S | V | S | E | I | Q | L | L | H | N | L | G | K | H | L | N | S | $ |
| 141 | 135 | H- | S | V | S | E | I | Q | L | L | H | N | L | G | K | $ | L | N | S | $ |
| 142 | 136 | H- | S | V | S | E | I | Q | L | L | H | N | L | G | $ | H | L | N | $ | L |
| 143 | 137 | H- | S | V | S | E | I | Q | L | L | H | N | L | G | K | H | L | N | S | $ |
| 144 | 138 | H- | Aib | V | Aib | E | I | Q | L | L | H | Q | hR | A | K | $ | I | Q | D | $ |
| 145 | 139 | H- | Aib | V | Aib | E | I | Q | L | L | H | Q | hR | A | K | $ | I | Q | D | $ |
| 146 | 140 | H- | Aib | V | Aib | E | I | Q | L | L | H | Q | hR | A | $ | W | I | Q | $ | L |
| 147 | 141 | H- | Aib | V | Aib | E | I | Q | L | L | H | Q | hR | A | K | W | I | Q | D | $ |
| 148 | 142 | H- | S | V | S | E | I | Q | L | L | H | N | L | G | K | H | L | N | S | $ |
| 149 | 143 | H- | S | V | S | E | I | Q | L | L | H | N | L | G | K | H | L | N | S | $r5 |

| SEQ | | | | | | | | | | | | | | | | | | | |
|---|---|---|---|---|---|---|---|---|---|---|---|---|---|---|---|---|---|---|---|
| ID | 19 | 20 | 21 | 22 | 23 | 24 | 25 | 26 | 27 | 28 | 29 | 30 | 31 | 32 | 33 | 34 | 35 | 36 | 37 |
| 8 | E | R | V | E | W | L | R | K | K | L | $ | D | V | H | $ | F | | | -NH₂ |
| 9 | E | R | V | E | W | L | R | K | K | $ | Q | D | V | $ | N | F | | | -NH₂ |
| 10 | E | R | V | E | W | L | R | K | $ | L | Q | D | $ | H | N | F | | | -NH₂ |
| 11 | E | R | V | E | W | L | R | $ | K | L | Q | $ | V | H | N | F | | | -NH₂ |
| 12 | E | R | V | E | W | L | $ | K | K | L | $ | D | V | H | N | F | | | -NH₂ |
| 13 | E | R | V | E | W | $ | R | K | K | $ | Q | D | V | H | N | F | | | -NH₂ |
| 14 | E | R | V | $ | W | L | R | $ | K | L | Q | D | V | H | N | F | | | -NH₂ |
| 15 | E | R | $ | E | W | L | $ | K | K | L | Q | D | V | H | N | F | | | -NH₂ |
| 16 | E | R | V | $ | W | L | R | K | K | L | Q | D | V | H | N | F | | | -NH₂ |
| 17 | E | R | $ | E | W | L | R | K | K | L | Q | D | V | H | N | F | | | -NH₂ |
| 18 | $ | R | V | E | W | L | R | K | K | L | Q | D | V | H | N | F | | | -NH₂ |
| 19 | E | R | V | E | W | L | R | K | K | L | Q | D | V | H | N | F | | | -NH₂ |
| 20 | E | R | V | E | W | L | R | K | K | L | Q | D | V | H | N | F | | | -NH₂ |
| 21 | E | R | V | E | W | L | R | K | K | L | Q | D | V | H | N | F | | | -NH₂ |
| 22 | E | R | V | E | W | L | R | K | K | L | Q | D | V | H | N | F | | | -NH₂ |
| 23 | E | R | V | E | W | L | R | K | K | L | Q | D | V | H | N | F | | | -NH₂ |
| 24 | E | R | V | E | W | L | R | K | K | L | Q | D | V | H | N | F | | | -NH₂ |
| 25 | E | R | V | E | W | L | R | K | K | L | Q | D | V | H | N | F | | | -NH₂ |
| 26 | E | R | V | E | W | L | R | K | K | L | Q | D | V | H | N | F | | | -NH₂ |
| 27 | E | R | V | E | W | L | R | K | K | L | Q | D | V | H | N | F | | | -NH₂ |
| 28 | E | R | V | E | W | L | R | K | K | L | Q | D | V | H | N | F | | | -NH₂ |
| 29 | E | R | V | E | W | L | R | K | K | L | Q | D | V | H | N | F | | | -NH₂ |
| 30 | E | R | V | E | W | L | R | K | K | L | Q | D | V | H | N | F | | | -NH₂ |
| 31 | E | R | V | E | W | L | R | K | K | L | Q | D | V | H | N | F | | | -NH₂ |
| 32 | E | R | V | E | W | L | R | K | K | L | Q | D | V | H | N | F | | | -NH₂ |
| 33 | E | R | V | E | W | L | R | K | K | L | Q | D | V | H | N | F | | | -NH₂ |
| 34 | E | R | V | E | W | L | R | $r8 | K | L | Q | D | V | H | $ | F | | | -NH₂ |
| 35 | E | R | V | E | W | L | $r8 | K | K | L | Q | D | V | $ | N | F | | | -NH₂ |
| 36 | E | R | V | E | W | L | $r8 | K | K | L | Q | D | V | $ | N | F | | | -NH₂ |
| 37 | E | R | V | E | W | $r8 | R | K | K | L | Q | D | $ | H | N | F | | | -NH₂ |
| 38 | E | R | V | E | W | $r8 | R | K | K | L | Q | D | $ | H | N | F | | | -NH₂ |
| 39 | E | R | V | $r8 | W | L | R | K | K | L | $ | D | V | H | N | F | | | -NH₂ |
| 40 | E | R | $r8 | E | W | L | R | K | K | $ | Q | D | V | H | N | F | | | -NH₂ |
| 41 | $r8 | R | V | E | W | L | R | $ | K | L | Q | D | V | H | N | F | | | -NH₂ |
| 42 | E | R | V | E | W | L | $ | K | K | L | Q | D | V | H | N | F | | | -NH₂ |
| 43 | E | R | V | E | W | L | $ | K | K | L | Q | D | V | H | N | F | | | -NH₂ |
| 44 | E | R | V | E | W | $ | R | K | K | L | Q | D | V | H | N | F | | | -NH₂ |
| 45 | E | R | V | E | W | $ | R | K | K | L | Q | D | V | H | N | F | | | -NH₂ |
| 46 | E | R | V | $ | W | L | R | K | K | L | Q | D | V | H | N | F | | | -NH₂ |
| 47 | E | R | V | $ | W | L | R | K | K | L | Q | D | V | H | N | F | | | -NH₂ |
| 48 | E | R | $ | E | W | L | R | K | K | L | Q | D | V | H | N | F | | | -NH₂ |
| 49 | E | R | $ | E | W | L | R | K | K | L | Q | D | V | H | N | F | | | -NH₂ |
| 50 | $ | R | V | E | W | L | R | K | K | L | Q | D | V | H | N | F | | | -NH₂ |
| 51 | E | R | V | E | W | L | R | K | K | L | Q | D | V | H | N | F | | | -NH₂ |
| 52 | E | R | V | E | W | L | R | K | K | L | Q | D | V | H | N | F | | | -NH₂ |
| 53 | E | R | V | E | W | L | R | K | K | L | Q | D | V | H | N | F | | | -NH₂ |
| 54 | E | R | V | E | W | L | R | K | K | L | Q | D | V | H | N | F | | | -NH₂ |
| 55 | E | R | V | E | W | L | R | K | K | L | Q | D | V | H | N | F | | | -NH₂ |
| 56 | E | R | V | E | W | L | R | K | K | L | Q | D | V | H | N | F | | | -NH₂ |
| 57 | E | R | V | E | W | L | R | K | K | L | Q | D | V | H | N | F | | | -NH₂ |
| 58 | E | R | V | E | W | L | R | K | K | L | Q | D | V | H | N | F | | | -NH₂ |
| 59 | E | R | V | E | W | L | R | K | K | L | Q | D | V | H | N | F | | | -NH₂ |
| 60 | E | R | V | E | W | L | R | K | K | L | Q | D | V | H | N | F | | | -NH₂ |
| 61 | E | R | V | E | W | L | R | K | K | L | Q | D | V | H | N | F | | | -NH₂ |
| 62 | E | R | V | E | W | L | R | K | K | L | Q | D | V | H | N | F | | | -NH₂ |
| 63 | E | R | V | E | W | L | R | $ | K | L | Q | $ | V | H | N | F | | | -NH₂ |
| 64 | E | R | V | E | W | L | R | $ | K | L | Q | $ | V | H | N | F | | | -NH₂ |
| 65 | E | R | V | E | W | L | R | $ | K | L | Q | $ | V | H | N | F | | | -NH₂ |
| 66 | E | R | V | $ | W | L | R | $ | K | L | Q | $ | V | H | N | F | | | -NH₂ |
| 67 | E | R | V | $ | W | L | R | $ | K | L | Q | D | V | H | N | F | | | -NH₂ |
| 68 | E | R | V | $ | W | L | R | $ | K | L | Q | D | V | H | N | F | | | -NH₂ |
| 69 | R | R | R | F | W | L | H | $ | L | I | A | $ | I | H | T | A | E | Y | -NH₂ |
| 70 | R | R | R | F | W | L | H | $ | L | I | A | $ | I | H | T | A | E | Y | -NH₂ |
| 71 | R | R | R | $ | W | L | H | $ | L | I | A | $ | I | H | T | A | E | Y | -NH₂ |
| 72 | R | R | R | $ | W | L | H | $ | L | I | A | E | I | H | T | A | E | Y | -NH₂ |
| 73 | R | R | R | $ | W | L | H | $ | L | I | A | E | I | H | T | A | E | Y | -NH₂ |
| 74 | R | R | R | $ | W | L | H | $ | L | I | A | E | I | H | T | A | E | Y | -NH₂ |
| 75 | R | R | R | $ | W | L | H | $ | L | I | A | E | I | H | T | A | E | Y | -NH₂ |
| 76 | E | R | V | $ | W | L | R | $ | K | L | Q | D | V | H | N | Y | | | -NH₂ |
| 77 | R | R | R | $ | W | L | R | $ | K | L | Q | D | V | H | N | Y | | | -NH₂ |
| 78 | R | R | R | $ | W | L | R | $ | K | L | Q | D | V | H | N | Y | | | -NH₂ |
| 79 | E | R | V | $ | W | L | R | $ | K | L | Q | D | V | H | N | Y | | | -NH₂ |
| 80 | E | R | V | $ | W | L | R | $ | K | L | Q | D | V | H | N | Y | | | -NH₂ |
| 81 | E | R | V | $ | W | L | R | $ | K | L | Q | D | V | H | N | Y | | | -NH₂ |
| 82 | R | R | R | $ | W | L | H | $ | L | I | A | E | I | H | T | A | E | Y | -NH₂ |
| 83 | R | R | R | F | W | L | H | H | L | I | A | E | I | H | T | A | E | Y | -NH₂ |
| 84 | R | R | R | $ | W | L | H | $ | L | I | A | E | I | H | T | A | E | Y | -NH₂ |
| 85 | R | R | R | $ | W | L | H | $ | L | I | A | E | I | H | T | A | | | -NH₂ |
| 86 | R | R | R | F | W | $ | H | H | L | $ | A | E | I | H | T | A | E | Y | -NH₂ |
| 87 | R | R | R | F | W | L | $ | H | L | I | $ | E | I | H | T | A | E | Y | -NH₂ |
| 88 | R | R | R | F | W | L | H | $ | L | I | A | $ | I | H | T | A | E | Y | -NH₂ |
| 89 | R | R | R | F | W | L | H | H | $ | I | A | E | $ | H | T | A | E | Y | -NH₂ |
| 90 | R | R | R | F | W | L | $r8 | H | L | I | A | E | I | H | T | A | E | Y | -NH₂ |
| 91 | R | R | R | F | W | L | $r8 | H | L | I | A | E | I | $ | T | A | E | Y | -NH₂ |
| 92 | R | R | R | F | W | $ | H | H | L | $ | A | E | I | $ | T | A | E | Y | -NH₂ |
| 93 | R | R | R | $ | W | L | H | $ | L | I | A | E | I | H | T | A | E | Y | -NH₂ |
| 94 | R | R | R | $ | W | L | H | $ | L | I | A | E | I | H | T | A | E | Y | -NH₂ |
| 95 | R | R | R | $ | W | L | H | $ | L | I | A | E | I | H | T | A | E | Y | -NH₂ |
| 96 | R | R | R | $ | W | L | H | $ | L | I | A | E | I | H | T | A | E | Y | -NH₂ |
| 97 | R | R | R | $ | W | L | H | $ | L | I | A | E | I | H | T | A | E | Y | -NH₂ |
| 98 | R | R | R | $ | W | L | H | $ | L | I | A | E | I | H | T | A | E | Y | -NH₂ |
| 99 | R | R | R | $ | W | L | H | $ | L | I | A | E | I | H | T | A | E | Y | -NH₂ |
| 100 | R | R | R | $ | W | L | H | $ | L | I | A | E | I | H | T | A | E | Y | -NH₂ |
| 101 | R | R | R | $ | W | L | H | $ | L | I | A | E | I | H | T | A | E | Y | -NH₂ |
| 102 | R | R | R | $ | W | L | H | $ | L | I | A | E | I | H | T | A | E | Y | -NH₂ |
| 103 | R | R | R | $ | W | L | H | $ | L | I | A | E | I | H | T | A | E | Y | -NH₂ |
| 104 | R | R | R | $ | W | L | H | $ | L | I | A | E | I | H | T | A | E | Y | -NH₂ |
| 105 | R | R | R | $ | W | L | H | $ | L | I | A | E | I | H | T | A | E | Y | -NH₂ |
| 106 | R | R | R | $ | W | L | H | $ | L | I | A | E | I | H | T | A | E | Y | -NH₂ |
| 107 | R | R | R | $ | W | L | H | $ | L | I | A | E | I | H | T | A | E | Y | -NH₂ |
| 108 | R | R | R | $ | W | L | H | $ | L | I | A | E | I | H | T | A | E | Y | -NH₂ |
| 109 | S | R | Q | $ | W | L | H | $ | Q | I | A | N | I | H | T | A | E | Y | -NH₂ |
| 110 | S | R | Q | $ | W | L | H | $ | L | I | A | E | I | H | T | A | E | Y | -NH₂ |
| 111 | R | R | R | $ | W | L | R | $ | F | I | A | E | I | H | T | A | E | Y | -NH₂ |
| 112 | R | R | R | $ | W | L | R | $ | Y | I | A | E | I | H | T | A | E | Y | -NH₂ |
| 113 | R | R | R | $ | W | L | W | $ | L | I | A | E | I | H | T | A | E | Y | -NH₂ |
| 114 | R | R | R | $ | W | L | Y | $ | L | I | A | E | I | H | T | A | E | Y | -NH₂ |
| 115 | R | R | R | $ | W | L | F | $ | L | I | A | E | I | H | T | A | E | Y | -NH₂ |
| 116 | R | R | R | $ | W | L | H | $ | L | I | A | E | I | 2 | T | A | E | Y | -NH₂ |
| | | | | | | | | | | | | | | Pal | | | | | |
| 117 | R | R | R | $ | W | L | H | $ | L | I | A | E | I | H | T | A | E | Y | -NH₂ |
| 118 | E | R | V | St | W | L | R | $r5 | K | V | Q | D | V | H | N | F | | | -NH₂ |
| 119 | E | R | V | St | W | L | R | $ | K | V | Q | D | V | H | N | F | | | -NH₂ |
| 120 | R | R | R | $ | F | L | H | H | L | I | A | E | I | H | T | A | E | Y | -NH₂ |
| 121 | R | R | R | $ | W | L | H | $ | L | I | A | E | I | H | T | A | E | Y | -NH₂ |
| 122 | R | R | R | St | W | L | H | $r5 | L | I | A | E | I | H | T | A | E | Y | -NH₂ |
| 123 | R | R | R | $r5 | W | L | H | H | L | I | A | E | I | H | T | A | E | Y | -NH₂ |
| 124 | R | R | R | $ | W | L | H | H | L | I | A | E | I | H | T | A | E | Y | -NH₂ |
| 125 | R | R | R | $ | W | L | H | H | L | I | A | E | I | H | T | A | E | Y | -NH₂ |
| 126 | R | R | R | F | W | L | H | H | L | I | A | E | I | H | T | A | E | Y | -NH₂ |
| 127 | R | R | R | F | W | $ | H | H | L | I | A | E | I | H | T | A | E | Y | -NH₂ |
| 128 | E | R | R | E | W | L | R | K | K | L | $ | D | V | H | $ | F | | | -NH₂ |
| 129 | E | R | R | E | W | L | R | K | K | L | $ | D | V | H | $ | F | | | -NH₂ |
| 130 | E | R | R | E | W | L | R | K | K | L | $ | D | V | H | $ | F | | | -NH₂ |
| 131 | E | R | V | E | W | L | R | $ | K | L | Q | $ | V | H | N | F | | | -NH₂ |
| 132 | E | R | V | E | W | L | R | $ | K | L | Q | $ | V | H | N | F | | | -NH₂ |
| 133 | E | R | V | $ | W | L | R | $ | K | L | Q | D | V | H | N | F | | | -NH₂ |
| 134 | E | R | V | E | W | $ | R | K | K | $ | Q | D | V | H | N | F | | | -NH₂ |
| 135 | E | R | V | E | W | $ | R | K | K | $ | Q | D | V | H | N | F | | | -NH₂ |
| 136 | E | R | V | E | W | $ | R | K | K | $ | Q | D | V | H | N | F | | | -NH₂ |
| 137 | E | R | V | $ | W | L | R | $ | K | L | Q | D | V | H | N | F | | | -NH₂ |
| 138 | E | R | V | E | W | L | R | $ | K | L | Q | V | V | H | N | F | | | -NH₂ |
| 139 | E | R | V | E | W | L | R | $ | K | L | Q | $ | V | H | N | F | | | -NH₂ |
| 140 | E | R | V | E | W | L | R | $ | K | L | Q | $ | V | H | N | F | | | -NH₂ |
| 141 | E | R | V | $ | W | L | R | $ | K | L | Q | $ | V | H | N | F | | | -NH₂ |
| 142 | E | R | V | $ | W | L | R | $ | K | L | Q | D | V | H | N | F | | | -NH₂ |
| 143 | E | R | V | $ | W | L | R | $ | K | L | Q | D | V | H | N | F | | | -NH₂ |
| 144 | R | R | R | F | W | L | H | $ | L | I | A | $ | I | H | T | F | | | -NH₂ |
| 145 | R | R | R | $ | W | L | H | $ | L | I | A | $ | I | H | T | A | E | Y | -NH₂ |
| 146 | R | R | R | $ | W | L | H | $ | L | I | A | E | I | H | T | A | E | Y | -NH₂ |
| 147 | R | R | R | $ | W | L | H | $ | L | I | A | E | I | H | T | A | E | Y | -NH₂ |
| 148 | E | R | V | St | W | L | R | $r5 | K | L | Q | D | V | H | T | A | E | Y | -NH₂ |
| 149 | E | R | V | St | W | L | R | $ | K | L | Q | D | V | H | T | A | E | Y | -NH₂ |

Table 1b
Peptidomimetic Macrocyles

| SEQ | | | | | | | | | | | | | | | | | | | | |
|---|---|---|---|---|---|---|---|---|---|---|---|---|---|---|---|---|---|---|---|---|
| ID | SP# | 0 | 1 | 2 | 3 | 4 | 5 | 6 | 7 | 8 | 9 | 10 | 11 | 12 | 13 | 14 | 15 | 16 | 17 | 18 |
| 150 | 144 | H- | | | | | | | L | L | H | N | L | G | K | $ | I | Q | D | $ |
| 151 | 145 | H- | | | | | | | L | L | H | D | K | G | K | $ | I | Q | D | $ |
| 152 | 146 | H- | | | | | | | F | L | H | Q | hR | A | K | $ | I | Q | D | $ |
| 153 | 147 | H- | | | | | | | F4 | L | H | Q | hR | A | K | $ | I | Q | D | $ |
| | | | | | | | | | Cl | | | | | | | | | | | |
| 154 | 148 | H- | | | | | | | L | Nle | H | Q | hR | A | K | $ | I | Q | D | $ |
| 155 | 149 | H- | | | | | | | L | L | H | Q | hR | A | K | $ | I | Q | D | $ |
| 156 | 150 | H- | | | | | | | L | L | H | Q | hR | w | K | $ | I | Q | D | $ |
| 157 | 151 | H- | | | | | | | F | Nle | H | Q | hR | A | K | $ | I | Q | D | $ |
| 158 | 152 | H- | | | | | | | L | L | H | A | hR | A | K | $ | I | Q | D | $ |
| 159 | 153 | H- | | | | | | | L | L | H | D | hR | A | K | $ | I | Q | D | $ |
| 160 | 154 | H- | | | | | | | L | L | H | Q | hR | A | S | $ | I | Q | D | $ |
| 161 | 155 | H- | | | | | | | L | $ | H | Q | hR | $ | K | W | I | Q | D | L |
| 162 | 156 | H- | | | | | | | $ | L | H | Q | $ | A | K | W | I | Q | D | L |
| 163 | 157 | Hep- | | | | | | | L | L | H | $ | V | A | K | W | I | Q | D | L |
| 164 | 158 | H- | | | | | | | L | L | H | Q | hR | A | K | W | I | Q | D | L |
| 165 | 159 | H- | | | | | | | $r8 | L | H | N | L | G | K | $ | L | N | S | L |
| 166 | 160 | H- | | | | | | | L | L | H | Q | R | A | K | W | I | Q | D | $ |
| 167 | 161 | Ac- | | | | | | | L | L | H | Q | R | A | K | W | I | Q | D | $ |
| 168 | 162 | H- | | | | | | | L | L | H | Q | R | A | K | W | I | Q | D | $ |
| 169 | 163 | H- | | | | | | | L | L | H | Q | R | A | K | W | I | Q | D | $ |
| 170 | 164 | H- | | | | | | | L | L | H | Q | R | A | K | W | I | Q | D | $ |
| 171 | 165 | H- | | | | | | | L | L | H | Q | R | A | K | W | I | Q | D | $ |
| 172 | 166 | H- | | | | | | | L | L | H | Q | R | A | K | W | I | Q | D | $ |
| 173 | 167 | H- | | | | | | | L | L | H | Q | R | A | K | W | I | Q | D | $ |
| 174 | 168 | H- | | | | | | | L | L | H | Q | R | A | K | W | I | Q | D | $ |
| 175 | 169 | H- | | | | | | | L | L | H | Q | R | A | K | W | I | Q | D | $ |
| 176 | 170 | H- | | | | | | | L | L | H | Q | R | A | K | W | I | Q | D | $ |
| 177 | 171 | H- | | | | | | | L | L | H | Q | R | A | K | W | I | Q | D | $ |
| 178 | 172 | H- | | | | | | | L | L | H | Q | R | A | K | W | I | Q | D | $ |
| 179 | 173 | H- | | | | | | | L | L | H | Q | R | A | K | W | I | Q | D | $ |
| 180 | 174 | H- | | | | | | | L | L | H | Q | R | A | K | W | I | Q | D | $ |
| 181 | 175 | H- | | | | | | | L | L | H | Q | R | A | K | W | I | Q | D | $ |
| 182 | 176 | H- | | | | | | | L | L | H | Q | R | A | K | W | I | Q | D | $ |
| 183 | 177 | H- | | | | | | | L | L | H | Q | R | A | K | W | I | Q | D | $ |
| 184 | 178 | H- | | | | | | | L | L | H | Q | R | A | K | W | I | Q | D | $ |
| 185 | 179 | H- | | | | | | | L | L | H | Q | R | A | K | W | I | Q | D | $ |
| 186 | 180 | H- | | | | | | | L | L | H | Q | R | A | K | W | I | Q | D | $ |
| 187 | 181 | H- | | | | | | | L | L | H | Q | R | A | K | W | I | Q | D | $ |
| 188 | 182 | H- | | | | | | | L | L | H | Q | R | A | K | W | I | Q | D | $ |
| 189 | 183 | H- | | | | | | | L | L | H | Q | R | A | K | W | I | Q | D | $ |
| 190 | 184 | H- | | | | | | | L | L | H | Q | R | A | K | W | I | Q | D | $ |
| 191 | 185 | H- | | | | | | | L | L | H | Q | R | A | K | W | I | Q | D | $ |
| 192 | 186 | H- | | | | | | | L | L | H | Q | R | A | K | W | I | Q | D | $ |
| 193 | 187 | H- | | | | | | | L | L | H | Q | R | A | K | W | I | Q | D | $ |
| 194 | 188 | H- | | | | | | | L | L | H | Q | R | A | K | W | I | Q | D | $ |
| 195 | 189 | H- | | | | | | | L | L | H | Q | R | A | K | W | I | Q | D | $ |
| 196 | 190 | H- | | | | | | | L | L | H | Q | R | A | K | W | I | Q | D | $ |
| 197 | 191 | H- | | | | | | | L | L | H | Q | R | A | K | W | I | Q | D | $ |
| 198 | 192 | H- | | | | | | | L | L | H | Q | R | A | K | W | I | Q | D | $ |
| 199 | 193 | H- | | | | | | | L | L | H | Q | R | A | K | W | I | Q | D | $ |
| 200 | 194 | H- | | | | | | | L | L | H | Q | R | A | K | W | I | Q | D | $ |
| 201 | 195 | H- | | | | | | | L | L | H | Q | R | A | K | W | I | Q | D | $ |
| 202 | 196 | H- | | | | | | | L | L | H | Q | R | A | K | W | I | Q | D | $ |
| 203 | 197 | H- | | | | | | | L | L | H | Q | R | A | K | W | I | Q | D | $ |
| 204 | 198 | H- | | | | | | | L | L | H | Q | R | A | K | W | I | Q | D | $ |
| 205 | 199 | H- | | | | | | | L | L | H | Q | R | A | K | W | I | A | D | $ |
| 206 | 200 | H- | | | | | | | L | L | H | Q | R | A | K | W | I | E | D | $ |
| 207 | 201 | H- | | | | | | | L | L | H | Q | R | A | K | F | I | Q | D | $ |
| 208 | 202 | H- | | | | | | | L | L | H | Q | R | A | A | W | I | Q | D | $ |
| 209 | 203 | H- | | | | | | | L | L | H | A | R | A | K | W | I | Q | D | $ |
| 210 | 204 | H- | | | | | | | L | L | H | E | R | A | K | W | I | Q | D | $ |
| 211 | 205 | H- | | | | | | | L | L | H | D | R | A | K | W | I | Q | D | $ |
| 212 | 206 | H- | | | | | | | L | L | H | Q | R | A | K | W | I | Q | D | $ |
| 213 | 207 | H- | | | | | | | L | L | H | Q | R | A | K | W | I | Q | D | $ |
| 214 | 208 | H- | | | | | | | L | L | H | Q | R | A | K | W | I | Q | D | $ |
| 215 | 209 | H- | | | | | | | L | L | H | Q | hR | A | K | W | I | Q | D | $r5 |
| 216 | 210 | H- | | | | | | | L | L | H | Q | hR | A | K | W | I | Q | D | $r8 |
| 217 | 211 | H- | | | | | | | L | L | H | Q | Hr | A | K | W | I | Q | $r8 | L |
| 218 | 212 | H- | | | | | | | L | L | H | D | K | A | K | S | I | Q | D | $ |
| 219 | 213 | H- | | | | | | | L | L | H | D | K | A | K | S | I | Q | D | $ |
| 220 | 214 | H- | | | | | | | L | L | H | Q | R | A | K | W | L | N | S | $ |
| 221 | 215 | H- | | | | | | | L | L | H | Q | R | A | K | $ | I | Q | D | $ |
| 222 | 216 | H- | | | | | | | L | L | H | Q | hR | A | K | W | I | Q | D | $ |
| 223 | 217 | H- | | | | | I | Q | L | L | H | Q | R | A | K | W | I | Q | D | $ |
| 224 | 218 | Ac- | | | | | | | L | L | H | D | K | A | K | S | I | Q | D | $ |
| 225 | 219 | Ac- | | | | | | | L | L | H | D | K | A | K | S | I | Q | D | $ |
| 226 | 220 | Ac- | | | | | | | L | L | H | Q | R | A | K | W | L | N | S | $ |
| 227 | 221 | Ac- | | | | | | | L | L | H | Q | R | A | K | $ | I | Q | D | $ |
| 228 | 222 | Ac- | | | | | | | L | L | H | Q | hR | A | K | W | I | Q | D | $ |
| 229 | 223 | H- | | | | | | | L | L | H | Q | R | A | K | $ | I | Q | D | $ |
| 230 | 224 | H- | | | | | | | L | L | H | Q | R | A | $ | W | I | Q | S | L |
| 231 | 225 | H- | | | | | | | L | L | H | Q | R | A | K | $ | I | Q | D | $ |
| 232 | 226 | H- | | | | | | | L | L | H | $ | R | A | K | $ | I | Q | D | L |
| 233 | 227 | H- | | | | | | | L | L | H | $ | R | A | K | $ | I | Q | D | L |
| 234 | 228 | H- | | | | | | | L | L | $ | Q | R | A | $ | W | I | Q | D | L |
| 235 | 229 | H- | | | | | | | L | L | $ | Q | R | A | $ | W | I | Q | D | L |
| 236 | 230 | H- | | | | | | | L | L | H | Q | R | A | K | W | $ | Q | D | L |
| 237 | 231 | H- | | | | | | | L | L | H | Q | R | A | K | W | I | Q | D | $ |
| 238 | 232 | H- | | | | | | | L | L | $ | Q | R | A | $ | W | I | Q | D | $ |
| 239 | 233 | H- | | | | | | | L | L | $ | Q | R | A | $ | W | I | Q | D | $ |
| 240 | 234 | H- | | | | | | | L | L | H | Q | R | A | K | W | I | Q | D | $5a |
| | | | | | | | | | | | | | | | | | | | | 5 |
| 241 | 235 | H- | | | | | | | L | L | H | Q | R | A | K | W | I | Q | D | $5n |
| | | | | | | | | | | | | | | | | | | | | 3 |
| 242 | 236 | H- | | | | | | | L | L | H | Q | R | A | K | $5a | I | Q | D | $5n |
| | | | | | | | | | | | | | | | | 5 | | | | 3 |
| 243 | 237 | H- | | | | | | | L | L | H | Q | R | A | K | $5n | I | Q | D | $5a |
| | | | | | | | | | | | | | | | | 3 | | | | 5 |
| 244 | 238 | H- | | | | | | | L | L | H | Q | R | A | K | $5a | I | Q | D | $5n |
| | | | | | | | | | | | | | | | | 5 | | | | 3 |
| 245 | 239 | H- | | | | | | | L | L | H | Q | R | A | $5a | W | I | Q | $5n | L |
| | | | | | | | | | | | | | | | 5 | | | | 3 | |
| 246 | 240 | H- | | | | | | | L | L | H | Q | R | A | K | $ | L | N | S | $ |
| 247 | 241 | H- | | | | | | | L | L | H | Q | R | A | $ | W | L | N | $ | L |
| 248 | 242 | H- | | | | | | | L | L | H | Q | R | A | $ | W | L | N | $ | L |
| 249 | 243 | H- | | | | | | | L | L | H | Q | R | A | K | W | L | N | S | $ |
| 250 | 244 | H- | | | | | | | L | L | H | Q | R | A | K | $ | I | Q | D | $ |
| 251 | 245 | H- | | | | | | | L | L | H | Q | R | A | $ | W | I | Q | $ | L |
| 252 | 246 | H- | | | | | | | L | L | H | Q | R | A | $ | W | I | Q | $ | L |
| 253 | 247 | H- | | | | | | | L | L | H | Q | R | A | K | W | I | Q | D | $ |
| 254 | 248 | H- | | | | | | | L | L | H | Q | hR | A | K | $ | I | Q | D | $ |
| 255 | 249 | H- | | | | | | | L | L | H | Q | R | A | K | W | I | Q | D | $ |
| 256 | 250 | H- | | | | | | | L | L | H | Q | R | A | K | W | I | Q | D | $ |
| 257 | 251 | H- | | | | | | | L | L | H | Q | R | A | K | W | I | Q | D | $ |
| 258 | 252 | H- | | | | | | | L | L | H | Q | R | A | Cit | W | I | Q | D | $ |
| 259 | 253 | H- | | | | | | | L | L | H | Q | Cit | A | K | W | I | Q | D | $ |
| 260 | 254 | H- | | | | | | | L | L | H | Q | R | A | K | W | I | Q | D | $ |
| 261 | 255 | H- | | | | | | | L | L | H | Q | R | A | K | W | I | Q | D | $ |
| 262 | 256 | H- | | | | | | | L | L | H | Q | R | A | K | W | I | Q | D | $ |
| 263 | 257 | H- | | | | | | | L | L | H | Q | R | A | K | W | I | Q | D | $ |
| 264 | 258 | H- | | | | | | | L | L | H | Q | R | A | K | W | I | Q | D | $ |
| 265 | 259 | H- | | | | | | | L | L | H | Q | R | A | K | W | I | Q | D | $ |
| 266 | 260 | H- | | | | | | | L | L | H | Q | R | A | K | W | I | Q | D | $ |
| 267 | 261 | H- | | | | | | | L | L | H | Q | R | A | K | W | I | Q | D | $ |
| 268 | 262 | H- | | | | | | | L | L | H | Q | R | A | Cit | W | I | Q | D | $ |
| 269 | 263 | H- | | | | | | | L | L | H | Q | Cit | A | K | W | I | Q | D | $ |
| 270 | 264 | H- | | | | | | | L | L | H | Q | R | A | K | W | I | Q | D | $ |
| 271 | 265 | H- | | | | | | | L | L | H | Q | R | A | K | W | I | Q | D | $ |
| 272 | 266 | H- | | | | | | | L | L | H | Q | $/ | A | K | W | I | Q | D | $ |
| 273 | 267 | H- | | | | | | | L | L | H | Q | Nle | A | K | W | I | Q | D | $ |
| 274 | 268 | H- | | | | | | | L | L | H | Q | hR | A | K | W | I | Q | D | $ |
| 275 | 269 | H- | | | | | | | L | L | H | Q | hR | A | Kfa | W | I | Q | D | $ |
| | | | | | | | | | | | | | | | m | | | | | |
| 276 | 270 | H- | | | | | | | L | L | H | Q | hR | A | Kfa | W | I | Q | D | $ |
| | | | | | | | | | | | | | | | m | | | | | |
| 277 | 271 | H- | | | | | | | L | L | H | Q | hR | A | K | W | I | Q | D | $ |
| 278 | 272 | H- | | | | | | | L | L | H | Q | hR | A | K | W | I | Q | D | $ |
| 279 | 273 | Ac- | | | | | | | $/ | L | H | E | R | A | K | F | I | Q | D | $ |
| 280 | 274 | Ac- | | | | | | | L | $/ | H | E | R | A | K | F | I | Q | D | $ |
| 281 | 275 | Ac- | | | | | | | L | L | H | $/ | R | A | K | F | I | Q | D | $ |
| 282 | 276 | Ac- | | | | | | | L | L | H | Aib | R | A | K | F | I | Q | D | $ |
| 283 | 277 | Ac- | | | | | | | L | L | H | E | $/ | A | K | F | I | Q | D | $ |
| 284 | 278 | Ac- | | | | | | | L | L | H | E | Nle | A | K | F | I | Q | D | $ |
| 285 | 279 | Ac- | | | | | | | L | L | H | E | R | $/ | K | F | I | Q | D | $ |
| 286 | 280 | Ac- | | | | | | | L | L | H | E | R | Aib | K | F | I | Q | D | $ |
| 287 | 281 | Ac- | | | | | | | L | L | H | E | R | A | $/ | F | I | Q | D | $ |
| 288 | 282 | Ac- | | | | | | | L | L | H | E | R | A | Aib | F | I | Q | D | $ |
| 289 | 283 | Ac- | | | | | | | L | L | H | E | R | A | K | F | $/ | Q | D | $ |
| 290 | 284 | Ac- | | | | | | | L | L | H | E | R | A | K | F | I | Q | D | $ |
| 291 | 285 | Ac- | | | | | | | L | L | H | E | R | A | K | F | I | Q | D | $ |
| 292 | 286 | Ac- | | | | | | | L | L | H | E | R | A | K | F | I | Q | D | $ |
| 293 | 287 | Ac- | | | | | | | L | L | H | E | R | A | K | F | I | Q | D | $ |
| 294 | 288 | Ac- | | | | | | | L | L | H | E | R | A | K | F | I | Q | D | $ |
| 295 | 289 | Ac- | | | | | | | L | L | H | E | R | A | K | F | I | Q | D | $ |
| 296 | 290 | Ac- | | | | | | | L | L | H | E | R | A | K | F | I | Q | D | $ |
| 297 | 291 | Ac- | | | | | | | L | L | H | E | R | A | K | F | I | Q | D | $ |
| 298 | 292 | Ac- | | | | | | | L | L | H | E | R | A | K | F | I | Q | D | $ |
| 299 | 293 | Ac- | | | | | | | L | L | H | E | R | A | K | F | I | Q | D | $ |
| 300 | 294 | Ac- | | | | | | | L | L | H | E | R | A | K | F | I | Q | D | $ |
| 301 | 295 | Ac- | | | | | | | L | L | H | E | $/ | A | K | F | I | Q | D | $ |
| 302 | 296 | Ac- | | | | | | | L | L | H | E | $/ | A | K | F | I | Q | D | $ |
| 303 | 297 | Ac- | | | | | | | L | L | H | E | R | $/ | K | F | I | Q | D | $ |
| 304 | 297 | Ac- | | | | | | | L | L | H | E | R | $/ | K | F | I | Q | D | $ |
| 305 | 299 | Ac- | | | | | | | L | L | H | E | R | Aib | K | F | I | Q | D | $ |
| 306 | 300 | Ac- | | | | | | | L | L | H | Q | R | A | K | W | L | N | S | $ |
| 307 | 301 | Ac- | | | | | | | L | L | H | E | R | A | K | F | L | N | S | $ |
| 308 | 302 | Ac- | | | | | | | L | L | $ | Q | R | A | $ | W | I | Q | D | $ |
| 309 | 303 | Ac- | | | | | | | L | L | $ | Q | R | A | $ | W | I | Q | D | L |
| 310 | 304 | Ac- | | | | | | | L | L | $ | Q | R | A | $ | W | I | Q | D | $ |
| 311 | 305 | Ac- | | | | | | | L | L | $ | Q | R | A | $ | W | I | Q | D | L |
| 312 | 306 | Ac- | | | | | | | L | L | $ | Q | R | A | $ | W | I | Q | D | $ |
| 313 | 307 | Ac- | | | | | | | L | L | $ | Q | R | A | $ | W | I | Q | D | L |
| 314 | 308 | Ac- | | | | | | | L | L | $ | A | R | A | $ | W | I | Q | D | $ |
| 315 | 309 | Ac- | | | | | | | L | L | $ | A | R | A | $ | W | I | Q | D | L |
| 316 | 310 | Ac- | | | | | | | L | L | $ | Q | R | A | $ | W | I | A | D | $ |
| 317 | 311 | Ac- | | | | | | | L | L | $ | Q | R | A | $ | W | I | A | D | L |
| 318 | 312 | Ac- | | | | | | | L | L | $ | A | R | A | $ | W | I | A | D | $ |
| 319 | 313 | Ac- | | | | | | | L | L | $ | A | R | A | $ | W | I | A | D | L |
| 320 | 314 | Ac- | | | | | | | L | L | $ | Q | R | A | $ | W | I | A | D | $ |
| 321 | 315 | Ac- | | | | | | | L | L | $ | Q | R | A | $ | W | I | A | D | L |
| 322 | 316 | Ac- | | | | | | | L | L | $ | A | R | A | $ | W | I | A | D | $ |
| 323 | 317 | Ac- | | | | | | | L | L | $ | A | R | A | $ | W | I | A | D | L |
| 324 | 318 | Ac- | | | | | | | L | L | $ | A | R | A | $ | W | I | A | D | L |
| 325 | 319 | Ac- | | | | | | | L | L | H | Q | R | A | $ | W | I | Q | $ | L |
| 326 | 320 | Ac- | | | | | | | L | L | H | Q | R | A | $ | W | I | Q | $ | L |
| 327 | 321 | Ac- | | | | | | | L | L | H | A | R | A | $ | W | I | Q | $ | L |
| 328 | 322 | Ac- | | | | | | | L | L | H | A | R | A | $ | W | I | Q | $ | L |
| 329 | 323 | Ac- | | | | | | | L | L | H | Q | R | A | $ | W | I | A | $ | L |
| 330 | 324 | Ac- | | | | | | | L | L | H | Q | R | A | $ | W | I | A | $ | L |
| 331 | 325 | Ac- | | | | | | | L | L | H | A | R | A | $ | W | I | A | $ | L |
| 332 | 326 | Ac- | | | | | | | L | L | H | A | R | A | $ | W | I | A | $ | L |
| 333 | 327 | Ac- | | | | | | | L | L | $ | Q | R | A | $ | W | I | A | D | L |
| 334 | 328 | Ac- | | | | | | | L | L | $ | Q | R | A | $ | W | I | A | D | L |
| 335 | 329 | Ac- | | | | | | | L | L | $ | Q | R | A | $ | W | I | Q | D | $ |
| 336 | 330 | Ac- | | | | | | | L | L | $ | Q | R | A | $ | W | I | Q | D | L |
| 337 | 331 | Ac- | | | | | | | L | L | $ | Q | R | A | $ | W | I | Q | D | L |
| 338 | 332 | Ac- | | | | | | | L | L | $ | Q | R | A | $ | W | I | Q | D | L |
| 339 | 333 | Ac- | | | | | | | L | L | H | $ | R | A | $ | $ | I | Q | D | L |
| 340 | 334 | Ac- | | | | | | | L | L | $ | Q | R | A | $ | W | I | Q | D | L |
| 341 | 335 | Ac- | | | | | | | L | L | $ | Q | R | A | $ | W | I | Q | D | L |
| 342 | 336 | Ac- | | | | | | | L | L | $ | A | R | A | $ | W | I | Q | D | $ |
| 343 | 337 | Ac- | | | | | | | L | L | $ | A | R | A | $ | W | I | Q | D | L |
| 344 | 338 | Ac- | | | | | | | L | L | $ | A | R | A | $ | W | I | Q | D | L |
| 45 | 339 | Ac- | | | | | | | L | L | $ | Q | R | A | $ | W | I | A | D | $ |
| 346 | 340 | Ac- | | | | | | | L | L | $ | A | R | A | $ | W | I | A | D | $ |
| 347 | 341 | Ac- | | | | | | | L | L | $ | A | R | A | $ | W | I | A | D | L |
| 348 | 342 | Ac- | | | | | | | L | L | $ | Q | R | A | $ | W | I | Q | D | $ |
| 349 | 343 | Ac- | | | | | | | L | L | $ | Q | R | A | $ | W | I | Q | D | $ |
| 350 | 344 | Ac- | | | | | | | L | L | H | Q | R | A | $ | W | I | Q | $ | L |
| 351 | 345 | Ac- | | | | | | | L | L | $ | Q | R | A | $ | W | I | A | D | $ |
| 352 | 346 | Ac- | | | | | | | L | L | H | Q | R | A | $ | W | I | A | $ | L |
| 353 | 347 | Ac- | | | | | | | L | L | $ | A | R | A | $ | W | I | Q | D | $ |
| 354 | 348 | Ac- | | | | | | | L | L | H | A | R | A | $ | W | I | Q | $ | L |
| 355 | 349 | Ac- | | | | | | | L | L | $ | A | R | A | $ | W | I | A | D | $ |
| 356 | 350 | Ac- | | | | | | | L | L | H | A | R | A | $ | W | I | A | $ | L |
| 357 | 351 | Ac- | | | | | | | L | L | H | Q | R | A | K | W | I | Q | D | $ |
| 358 | 352 | Ac- | | | | | | | L | L | H | Q | R | A | K | W | I | Q | D | $ |
| 359 | 353 | Ac- | | | | | | | L | L | H | Q | R | A | K | W | I | Q | D | $ |
| 360 | 354 | Ac- | | | | | | | L | L | H | Q | R | A | K | W | I | Q | D | $ |
| 361 | 355 | Ac- | | | | | | | L | L | H | Q | R | A | K | W | I | Q | D | $ |
| 362 | 356 | Ac- | | | | | | | L | L | H | Q | R | A | K | W | I | Q | D | L |
| 363 | 357 | Ac- | | | | | | | L | L | H | Q | R | A | K | W | I | Q | D | $ |
| 364 | 358 | Ac- | | | | | | | L | L | H | Q | R | A | K | W | I | Q | D | $ |
| 365 | 359 | Ac- | | | | | | | L | L | $ | Q | R | A | $ | W | I | Q | D | L |
| 366 | 360 | Ac- | | | | | | | L | L | H | Q | R | A | $ | W | I | Q | $ | L |
| 367 | 361 | Ac- | | | | | | | L | L | $ | Q | R | A | $ | W | I | Q | D | $ |
| 368 | 362 | Ac- | | | | | | | L | L | H | Q | R | A | $ | W | I | Q | $ | L |
| 369 | 363 | Ac- | | | | | | | L | L | H | $ | R | A | K | $ | I | Q | D | L |
| 370 | 367 | Ac- | | | | | | | L | L | H | E | R | Aib | K | F | I | Q | D | $ |
| 371 | 368 | Ac- | | | | | | | L | L | H | E | Nle | Aib | K | F | I | Q | D | $ |
| 372 | 369 | Ac- | | | | | | | L | L | H | E | Nle | Aib | K | F | I | A | D | $ |
| 373 | 370 | Ac- | | | | | | | L | L | H | E | Leu | Aib | K | F | I | A | D | $ |
| 374 | 371 | Ac- | | | | | | | L | L | H | E | Ile | Aib | K | F | I | A | D | $ |
| 375 | 372 | Ac- | | | | | | | L | L | H | E | Lys | Aib | K | F | I | A | D | $ |
| 376 | 373 | Ac- | | | | | | | L | L | H | E | R | Aib | K | W | I | Q | D | $ |
| 377 | 374 | Ac- | | | | | | | L | L | H | E | Nle | Aib | K | W | I | Q | D | $ |
| 378 | 375 | Ac- | | | | | | | L | L | H | E | Nle | Aib | K | W | I | A | D | $ |
| 379 | 376 | Ac- | | | | | | | L | L | H | E | Leu | Aib | K | W | I | A | D | $ |
| 380 | 377 | Ac- | | | | | | | L | L | H | E | Ile | Aib | K | W | I | A | D | $ |
| 381 | 378 | Ac- | | | | | | | L | L | H | E | Lys | Aib | K | W | I | A | D | $ |
| 382 | 364 | Ac- | | | | | | | L | L | H | Q | R | A | K | W | L | N | S | $ |
| 383 | 365 | Ac- | | | | | | | L | L | H | Q | R | A | K | W | $r8 | N | S | L |
| 384 | 366 | Ac- | | | | | | | L | L | H | D | K | A | K | S | $r8 | Q | D | L |
| 385 | 379 | Ac- | | | | | | | L | L | H | D | K | A | K | S | $r8 | Q | D | L |
| 386 | 380 | Ac- | | | | | | | L | L | H | Q | R | A | K | W | $r8 | N | S | L |
| 387 | 381 | Ac- | | | | | | | L | L | H | Q | R | A | K | W | $r8 | N | S | L |
| 388 | 383 | Ac- | | | | | | | L | L | $ | Q | R | A | $ | W | L | N | S | $ |
| 388 | 583 | Ac- | | | | | | | L | L | $ | Q | R | A | $ | W | L | N | S | $ |
| 389 | 384 | Ac- | | | | | | | L | L | $ | D | K | A | $ | S | I | Q | D | $ |
| 389 | 584 | Ac- | | | | | | | L | L | $ | D | K | A | $ | S | I | Q | D | $ |
| 390 | 385 | Ac- | | | | | | | L | L | $ | Q | R | A | $ | W | L | N | S | $ |
| 390 | 585 | Ac- | | | | | | | L | L | $ | Q | R | A | $ | W | L | N | S | $ |
| 391 | 386 | Ac- | | | | | | | L | L | $ | D | K | A | $ | S | I | Q | D | $ |
| 391 | 586 | Ac- | | | | | | | L | L | $ | D | K | A | $ | S | I | Q | D | $ |
| 392 | 387 | Ac- | | | | | | | L | L | $ | Q | R | A | $ | W | I | Q | D | $ |
| 393 | 388 | Ac- | | | | | | | L | L | $ | Q | R | A | $ | W | L | N | S | $ |
| 394 | 389 | Ac- | | | | | | | L | L | H | Q | R | A | K | W | $ | N | S | L |
| 395 | 390 | Ac- | | | | | | | L | L | H | D | K | A | K | S | $ | Q | D | L |
| 396 | 391 | Ac- | | | | | | | L | L | H | D | K | A | K | S | $ | Q | D | L |
| 397 | 392 | Ac- | | | | | | | L | L | H | Q | R | A | K | W | $ | N | S | L |
| 398 | 393 | Ac- | | | | | | | L | L | H | Q | R | A | K | W | $ | N | S | L |
| 399 | 394 | Ac- | | | | | | | L | L | H | Q | R | Aib | K | W | I | Q | D | $ |
| 400 | 395 | Ac- | | | | | | | L | L | H | Q | L | Aib | K | W | I | Q | D | $ |
| 400 | 595 | Ac- | | | | | | | L | L | H | Q | Nle | Aib | K | W | I | Q | D | $ |
| 400 | 695 | Ac- | | | | | | | L | L | H | Q | K | Aib | K | W | I | Q | D | $ |
| 401 | 396 | Ac- | | | | | | | L | L | H | Q | R | A | K | W | $ | Q | D | L |
| 402 | 397 | Ac- | | | | | | | L | L | H | Q | R | A | K | W | $r8 | Q | D | L |
| 403 | 398 | Ac- | | | | | | | L | L | H | Q | R | A | K | W | $r8 | Q | D | L |
| 404 | 400 | Ac- | | | | | | | L | L | H | E | R | Aib | K | F | I | Q | D | $ |
| 405 | 401 | Ac- | | | | | | | L | L | H | E | Cit | Aib | K | F | I | A | D | $ |
| 406 | 402 | Ac- | | | | | | | L | L | H | E | R | Aib | K | W | I | Q | D | $ |
| 407 | 403 | Ac- | | | | | | | L | L | H | E | Cit | Aib | K | W | I | A | D | $ |
| 408 | 404 | Ac- | | | | | | | L | L | H | E | R | Aib | K | F | I | Q | D | $ |
| 409 | 405 | Ac- | | | | | | | L | L | H | Q | R | A | K | $ | I | Q | D | $ |
| 410 | 406 | Ac- | | | | | | | L | L | H | Q | R | A | K | W | $ | Q | D | L |
| 411 | 407 | Ac- | | | | | | | L | L | H | $ | R | A | K | $ | I | Q | D | L |
| 412 | 408 | Ac- | | | | | | | L | L | $ | Q | R | A | $ | W | I | Q | D | $ |
| 413 | 409 | Ac- | | | | | | | L | L | H | $ | L | A | K | $ | I | Q | D | L |
| 413 | 509 | Ac- | | | | | | | L | L | H | $ | Nle | A | K | $ | I | Q | D | L |
| 413 | 609 | Ac- | | | | | | | L | L | H | $ | K | A | K | $ | I | Q | D | L |
| 414 | 410 | Ac- | | | | | | | L | L | $ | Q | L | A | $ | W | I | Q | D | L |
| 414 | 510 | Ac- | | | | | | | L | L | $ | Q | Nle | A | $ | W | I | Q | D | L |
| 414 | 610 | Ac- | | | | | | | L | L | $ | Q | K | A | $ | W | I | Q | D | L |
| 415 | 411 | Ac- | | | | | | | L | L | $ | Q | L | A | $ | W | I | Q | D | $ |
| 415 | 511 | Ac- | | | | | | | L | L | $ | Q | Nle | A | $ | W | I | Q | D | $ |
| 415 | 611 | Ac- | | | | | | | L | L | $ | Q | K | A | $ | W | I | Q | D | $ |
| 416 | 412 | Ac- | | | | | | | L | L | H | Q | R | Aib | K | W | I | Q | D | $ |
| 417 | 413 | Ac- | | | | | | | L | L | H | Q | R | Aib | K | W | I | Q | D | $ |
| 418 | 414 | Ac- | | | | | | | L | L | H | Q | R | Aib | K | W | I | Q | D | $ |
| 419 | 415 | Ac- | | | | | | | L | L | H | Q | R | Aib | K | W | I | Q | D | $ |
| 420 | 416 | Ac- | | | | | | | L | L | H | Q | R | Aib | K | W | I | Q | D | $ |
| 421 | 417 | Ac- | | | | | | | L | L | H | Q | R | Aib | K | W | I | Q | D | $ |
| 422 | 418 | Ac- | | | | | | | L | L | H | Q | R | Aib | K | W | I | Q | D | $ |
| 423 | 419 | Ac- | | | | | | | L | L | H | Q | R | Aib | K | W | I | Q | D | $ |
| 424 | 420 | Ac- | | | | | | | L | L | H | Q | R | Aib | K | W | I | Q | D | $ |
| 425 | 421 | Ac- | | | | | | | L | L | H | Q | R | Aib | K | W | I | Q | D | $ |
| 426 | 422 | Ac- | | | | | | | L | L | H | Q | R | Aib | K | W | I | Q | D | $ |
| 427 | 423 | Ac- | | | | | | | L | L | H | Q | R | D- | K | W | I | Q | D | $ |
| | | | | | | | | | | | | | | Trp | | | | | | |
| 428 | 424 | Ac- | | | | | | | L | L | H | Q | R | Aib | K | W | $r8 | Q | D | L |
| 429 | 425 | Ac- | | | | | | | L | L | H | Q | R | Aib | K | W | $ | Q | D | L |
| 430 | 426 | Ac- | | | | | | | L | L | H | Q | R | Aib | K | W | $ | Q | D | L |
| 431 | 427 | Ac- | | | | | | | L | L | H | Q | R | Aib | K | W | $r8 | Q | D | L |
| 432 | 428 | Ac- | | | | | | | L | L | H | Q | R | A | K | W | I | Q | D | $ |
| 433 | 429 | Ac- | | | | | | | L | L | H | Q | R | A | K | D- | I | Q | D | $ |
| | | | | | | | | | | | | | | | | Trp | | | | |
| 434 | 430 | Ac- | | | | | | | L | L | H | Q | R | A | K | W | $r8 | Q | D | K |
| 435 | 431 | Ac- | | | | | | | L | L | $ | Q | R | A | $ | W | I | Q | D | $ |
| 435 | 531 | Ac- | | | | | | | L | L | $ | Q | R | A | $ | W | I | Q | D | $ |
| 436 | 432 | Ac- | | | | | | | L | L | $ | Q | R | A | $ | W | L | N | S | $ |
| 436 | 532 | Ac- | | | | | | | L | L | $ | Q | R | A | $ | W | L | N | S | $ |
| 437 | 433 | Ac- | | | | | | | L | L | $ | Q | R | A | $ | W | I | Q | D | $ |
| 438 | 434 | Ac- | | | | | | | L | L | H | Q | R | A | $ | W | I | Q | $ | L |
| 439 | 435 | Ac- | | | | | | | L | L | $ | Q | R | A | $ | W | I | Q | D | L |
| 440 | 436 | Ac- | | | | | | | L | L | $ | Q | R | A | $ | W | I | Q | D | L |
| 441 | 437 | Ac- | | | | | | | L | L | H | Q | R | A | $ | W | I | Q | $ | L |
| 442 | 438 | Ac- | | | | | | | L | L | H | Q | R | A | K | $ | I | Q | D | $ |
| 443 | 439 | Ac- | | | | | | | L | L | H | $ | R | A | K | $ | I | Q | D | L |
| 444 | 440 | Ac- | | | | | | | L | L | $ | Q | R | A | $ | W | I | Q | D | $ |
| 445 | 441 | Ac- | | | | | | | L | L | $ | Q | R | A | $ | W | I | Q | D | L |

| SEQ | | | | | | | | | | | | | | | | | | | |
|---|---|---|---|---|---|---|---|---|---|---|---|---|---|---|---|---|---|---|---|
| ID | 19 | 20 | 21 | 22 | 23 | 24 | 25 | 26 | 27 | 28 | 29 | 30 | 31 | 32 | 33 | 34 | 35 | 36 | 37 |
| 150 | R | R | R | $ | W | L | H | $ | L | I | A | E | I | H | T | A | E | Y | -NH₂ |
| 151 | R | R | R | $ | W | L | H | $ | L | I | A | E | I | H | T | A | E | Y | -NH₂ |
| 152 | R | R | R | $ | W | L | H | $ | L | I | A | E | I | H | T | A | E | Y | -NH₂ |
| 153 | R | R | R | $ | W | L | H | $ | L | I | A | E | I | H | T | A | E | Y | -NH₂ |
| 154 | R | R | R | $ | W | L | H | $ | L | I | A | E | I | H | T | A | E | Y | -NH₂ |
| 155 | R | R | R | $ | W | L | H | $ | L | I | A | E | I | H | T | A | E | Y | -NH₂ |
| 156 | R | R | R | $ | W | L | H | $ | L | I | A | E | I | H | T | A | E | Y | -NH₂ |
| 157 | R | R | R | $ | W | L | H | $ | L | I | A | E | I | H | T | A | E | Y | -NH₂ |
| 158 | R | R | R | $ | W | L | H | $ | L | I | A | E | I | H | T | A | E | Y | -NH₂ |
| 159 | R | R | R | $ | W | L | H | $ | L | I | A | E | I | H | T | A | E | Y | -NH₂ |
| 160 | R | R | R | $ | W | L | H | $ | L | I | A | E | I | H | T | A | E | Y | -NH₂ |
| 161 | R | R | R | $ | W | L | H | $ | L | I | A | E | I | H | T | A | E | Y | -NH₂ |
| 162 | R | R | R | $ | W | L | H | $ | L | I | A | E | I | H | T | A | E | Y | -NH₂ |
| 163 | R | R | R | $ | W | L | H | $ | L | I | A | E | I | H | T | A | E | Y | -NH₂ |
| 164 | R | R | R | $ | W | L | H | $ | L | I | A | E | I | H | T | A | E | Y | -NH₂ |
| 165 | E | R | V | E | W | L | R | K | K | L | Q | D | V | H | N | F | | | -NH₂ |
| 166 | R | R | R | $ | F | L | H | H | L | I | A | E | I | H | T | A | | | -NH₂ |
| 167 | R | R | R | $ | F | L | H | H | L | I | A | E | I | H | T | A | | | -NH₂ |
| 168 | R | R | R | $ | F | L | H | H | L | I | A | E | I | H | T | S | | | -NH₂ |
| 169 | R | R | R | $ | F | L | H | H | L | I | A | E | I | H | F | A | | | -NH₂ |
| 170 | R | R | R | $ | F | L | H | H | L | I | A | E | I | F | T | A | | | -NH₂ |
| 171 | R | R | R | $ | F | L | H | H | L | I | A | E | I | A | T | A | | | -NH₂ |
| 172 | R | R | R | $ | F | L | H | H | L | I | A | E | Nle | H | T | A | | | -NH₂ |
| 173 | R | R | R | $ | F | L | H | H | L | I | A | E | T | H | T | A | | | -NH₂ |
| 174 | R | R | R | $ | F | L | H | H | L | I | A | E | Cba | H | T | A | | | -NH₂ |
| 175 | R | R | R | $ | F | L | H | H | L | I | A | E | Cpg | H | T | A | | | -NH₂ |
| 176 | R | R | R | $ | F | L | H | H | L | I | A | A | I | H | T | A | | | -NH₂ |
| 177 | R | R | R | $ | F | L | H | H | L | I | E | E | I | H | T | A | | | -NH₂ |
| 178 | R | R | R | $ | F | L | H | H | L | I | S | E | I | H | T | A | | | -NH₂ |
| 179 | R | R | R | $ | F | L | H | H | L | I | A | E | I | H | T | A | | | -NH₂ |
| 180 | R | R | R | $ | F | L | H | H | L | Cba | A | E | I | H | T | A | | | -NH₂ |
| 181 | R | R | R | $ | F | L | H | H | L | Cha | A | E | I | H | T | A | | | -NH₂ |
| 182 | R | R | R | $ | F | L | H | H | L | Cpg | A | E | I | H | T | A | | | -NH₂ |
| 183 | R | R | R | $ | F | L | H | H | F | I | A | E | I | H | T | A | | | -NH₂ |
| 184 | R | R | R | $ | F | L | H | H | Nle | I | A | E | I | H | T | A | | | -NH₂ |
| 185 | R | R | R | $ | F | L | H | H | Y | I | A | E | I | H | T | A | | | -NH₂ |
| 186 | R | R | R | $ | F | L | H | H | H | I | A | E | I | H | T | A | | | -NH₂ |
| 187 | R | R | R | $ | F | L | H | H | hF | I | A | E | I | H | T | A | | | -NH₂ |
| 188 | R | R | R | $ | F | L | H | A | L | I | A | E | I | H | T | A | | | -NH₂ |
| 189 | R | R | R | $ | F | L | H | F | L | I | A | E | I | H | T | A | | | -NH₂ |
| 190 | R | R | R | $ | F | L | H | S | L | I | A | E | I | H | T | A | | | -NH₂ |
| 191 | R | R | R | $ | F | L | H | E | L | I | A | E | I | H | T | A | | | -NH₂ |
| 192 | R | R | R | $ | F | L | A | H | L | I | A | E | I | H | T | A | | | -NH₂ |
| 193 | R | R | R | $ | F | L | F | H | L | I | A | E | I | H | T | A | | | -NH₂ |
| 194 | R | R | R | $ | F | L | S | H | L | I | A | E | I | H | T | A | | | -NH₂ |
| 195 | R | R | R | $ | F | L | E | H | L | I | A | E | I | H | T | A | | | -NH₂ |
| 196 | R | R | R | $ | F | Cba | H | H | L | I | A | E | I | H | T | A | | | -NH₂ |
| 197 | R | R | R | $ | F | Cpg | H | H | L | I | A | E | I | H | T | A | | | -NH₂ |
| 198 | R | R | R | $ | W | L | H | H | L | I | A | E | I | H | T | A | | | -NH₂ |
| 199 | R | R | R | $ | 1Nal | L | H | H | L | I | A | E | I | H | T | A | | | -NH₂ |
| 200 | R | R | R | $ | 2Nal | L | H | H | L | I | A | E | I | H | T | A | | | -NH₂ |
| 201 | R | R | A | $ | F | L | H | H | L | I | A | E | I | H | T | A | | | -NH₂ |
| 202 | S | R | R | $ | F | L | H | H | L | I | A | E | I | H | T | A | | | -NH₂ |
| 203 | E | R | R | $ | F | L | H | H | L | I | A | E | I | H | T | A | | | -NH₂ |
| 204 | A | R | R | $ | F | L | H | H | L | I | A | E | I | H | T | A | | | -NH₂ |
| 205 | R | R | R | $ | F | L | H | H | L | I | A | E | I | H | T | A | | | -NH₂ |
| 206 | R | R | R | $ | F | L | H | H | L | I | A | E | I | H | T | A | | | -NH₂ |
| 207 | R | R | R | $ | F | L | H | H | L | I | A | E | I | H | T | A | | | -NH₂ |
| 208 | R | R | R | $ | F | L | H | H | L | I | A | E | I | H | T | A | | | -NH₂ |
| 209 | R | R | R | $ | F | L | H | H | L | I | A | E | I | H | T | A | | | -NH₂ |
| 210 | R | R | R | $ | F | L | H | H | L | I | A | E | I | H | T | A | | | -NH₂ |
| 211 | R | R | R | $ | F | L | H | H | L | I | A | E | I | H | T | A | | | -NH₂ |
| 212 | R | R | R | $ | F | L | H | H | L | I | A | E | I | | | | | | -NH₂ |
| 213 | R | R | R | $ | F | L | H | H | L | I | | | | | | | | | -NH₂ |
| 214 | R | R | R | $ | F | L | | | | | | | | | | | | | -NH₂ |
| 215 | R | R | R | St | W | L | H | $ | L | I | A | E | I | H | T | A | E | Y | -NH₂ |
| 216 | R | R | R | F | W | L | $ | H | L | I | A | E | I | H | T | A | E | Y | -NH₂ |
| 217 | R | R | R | F | W | $ | H | H | L | I | A | E | I | H | T | A | E | Y | -NH₂ |
| 218 | R | R | R | $ | W | L | R | K | K | L | Q | D | V | H | N | F | | | -NH₂ |
| 219 | R | R | R | $ | F | L | H | H | L | I | A | E | I | H | T | A | | | -NH₂ |
| 220 | R | R | V | $ | W | L | R | K | K | L | Q | D | V | H | N | F | | | -NH₂ |
| 221 | R | R | R | F | W | $ | H | H | L | $ | A | E | I | H | T | A | | | -NH₂ |
| 222 | R | R | R | $ | F | L | H | H | L | I | A | E | I | H | T | A | | | -NH₂ |
| 223 | R | R | R | $ | F | L | H | H | L | I | A | E | I | H | T | A | | | -NH₂ |
| 224 | R | R | R | $ | W | L | R | K | K | L | Q | D | V | H | N | F | | | -NH₂ |
| 225 | R | R | R | $ | F | L | H | H | L | I | A | E | I | H | T | A | | | -NH₂ |
| 226 | R | R | V | $ | W | L | R | K | K | L | Q | D | V | H | N | F | | | -NH₂ |
| 227 | R | R | R | F | W | $ | H | H | L | $ | A | E | I | H | T | A | | | -NH₂ |
| 228 | R | R | R | $ | F | L | H | H | L | I | A | E | I | H | T | A | | | -NH₂ |
| 229 | R | R | R | F | W | L | H | H | $ | I | A | E | $ | H | T | A | | | -NH₂ |
| 230 | R | R | R | F | W | L | H | H | $ | I | A | E | $ | H | T | A | | | -NH₂ |
| 231 | R | R | $ | F | W | L | $ | H | L | I | A | E | I | H | T | A | | | -NH₂ |
| 232 | R | R | R | F | W | $ | H | H | L | $ | A | E | I | H | T | A | | | -NH₂ |
| 233 | R | R | R | F | W | L | H | H | $ | I | A | E | $ | H | T | A | | | -NH₂ |
| 234 | R | R | R | F | W | $ | H | H | L | $ | A | E | I | H | T | A | | | -NH₂ |
| 235 | R | R | R | F | W | L | H | H | $ | I | A | E | $ | H | T | A | | | -NH₂ |
| 236 | $ | R | R | F | W | $ | H | H | L | $ | A | E | I | H | T | A | | | -NH₂ |
| 237 | R | R | R | $ | W | L | H | H | $ | I | A | E | $ | H | T | A | | | -NH₂ |
| 238 | R | R | R | $ | W | L | H | H | L | I | A | E | I | H | T | A | | | -NH₂ |
| 239 | R | R | R | $ | W | L | H | H | $ | I | A | E | $ | H | T | A | | | -NH₂ |
| 240 | R | R | R | $5n | F | L | H | H | L | I | A | E | I | H | T | A | | | -NH₂ |
| | | | | 3 | | | | | | | | | | | | | | | |
| 241 | R | R | R | $5a | F | L | H | H | L | I | A | E | I | H | T | A | | | -NH₂ |
| | | | | 5 | | | | | | | | | | | | | | | |
| 242 | R | R | R | $5n | W | L | H | $5a | L | I | A | E | I | H | T | A | | | -NH₂ |
| | | | | 3 | | | | 5 | | | | | | | | | | | |
| 243 | R | R | R | $5a | W | L | H | $5n | L | I | A | E | I | H | T | A | | | -NH₂ |
| | | | | 5 | | | | 3 | | | | | | | | | | | |
| 244 | R | R | R | F | W | $5a | H | H | L | $5n | A | E | I | H | T | A | | | -NH₂ |
| | | | | | | 5 | | | | 3 | | | | | | | | | |
| 245 | R | R | R | F | W | $5a | H | H | L | $5n | A | E | I | H | T | A | | | -NH₂ |
| | | | | | | 5 | | | | 3 | | | | | | | | | |
| 246 | R | R | R | E | W | $ | R | K | K | $ | Q | D | V | H | N | F | | | -NH₂ |
| 247 | R | R | R | E | W | $ | R | K | K | $ | Q | D | V | H | N | F | | | -NH₂ |
| 248 | R | R | R | $ | W | L | R | $ | K | L | Q | D | V | H | N | F | | | -NH₂ |
| 249 | R | R | R | $ | W | L | R | K | K | L | Q | D | V | H | N | F | | | -NH₂ |
| 250 | R | R | R | E | W | $ | R | K | K | $ | Q | D | V | H | N | F | | | -NH₂ |
| 251 | R | R | R | E | W | $ | R | K | K | $ | Q | D | V | H | N | F | | | -NH₂ |
| 252 | R | R | R | $ | W | L | R | $ | K | L | Q | D | V | H | N | F | | | -NH₂ |
| 253 | R | R | R | $ | W | L | R | K | K | L | Q | D | V | H | N | F | | | -NH₂ |
| 254 | R | A | R | $ | W | L | R | $ | L | I | A | E | I | H | T | A | | | -NH₂ |
| 255 | R | R | Cit | $ | F | L | H | H | L | I | A | E | I | H | T | A | | | -NH₂ |
| 256 | R | Cit | R | $ | F | L | H | H | L | I | A | E | I | H | T | A | | | -NH₂ |
| 257 | Cit | R | R | $ | F | L | H | H | L | I | A | E | I | H | T | A | | | -NH₂ |
| 258 | R | R | R | $ | F | L | H | H | L | I | A | E | I | H | T | A | | | -NH₂ |
| 259 | R | R | R | $ | F | L | H | H | L | I | A | E | I | H | T | A | | | -NH₂ |
| 260 | R | R | R | $ | F | L | H | H | L | I | A | F | I | H | T | A | | | -NH₂ |
| 261 | R | R | R | $ | F | L | H | H | L | I | A | E | I | Y | T | A | | | -NH₂ |
| 262 | R | R | R | $ | W | L | R | K | Cit | L | Q | D | V | H | N | F | | | -NH₂ |
| 263 | R | R | R | $ | W | L | R | Cit | L | L | Q | D | V | H | N | F | | | -NH₂ |
| 264 | R | R | R | $ | W | L | Cit | K | L | L | Q | D | V | H | N | F | | | -NH₂ |
| 265 | R | R | Cit | $ | W | L | R | K | L | L | Q | D | V | H | N | F | | | -NH₂ |
| 266 | R | Cit | R | $ | W | L | R | K | L | L | Q | D | V | H | N | F | | | -NH₂ |
| 267 | Cit | R | R | $ | W | L | R | K | L | L | Q | D | V | H | N | F | | | -NH₂ |
| 268 | R | R | R | $ | W | L | R | K | L | L | Q | D | V | H | N | F | | | -NH₂ |
| 269 | R | R | R | $ | W | L | R | K | L | L | Q | D | V | H | N | F | | | -NH₂ |
| 270 | R | R | R | $ | W | L | R | K | L | L | Q | F | V | H | N | F | | | -NH₂ |
| 271 | R | R | R | $ | W | L | R | K | L | L | Q | D | V | Y | N | F | | | -NH₂ |
| 272 | R | R | R | $ | F | L | H | H | L | I | A | E | I | H | T | A | | | -NH₂ |
| 273 | R | R | R | $ | F | L | H | H | L | I | A | E | I | H | T | A | | | -NH₂ |
| 274 | R | A | R | $ | A | A | H | H | L | I | A | E | I | H | T | A | E | Y | -NH₂ |
| 275 | R | R | R | $ | F | L | H | H | L | I | A | E | I | H | T | A | E | Y | -NH₂ |
| 276 | R | R | R | $ | F | L | H | H | L | I | A | E | I | H | T | A | E | Y | -NH₂ |
| 277 | R | R | R | $ | F | L | H | H | L | I | A | E | I | H | T | A | E | Y | -NH₂ |
| 278 | R | R | R | $ | F | L | H | H | L | I | A | E | I | H | T | A | E | Y | -NH₂ |
| 279 | R | R | R | $ | F | L | H | H | L | I | A | E | I | H | T | A | | | -NH₂ |
| 280 | R | R | R | $ | F | L | H | H | L | I | A | E | I | H | T | A | | | -NH₂ |
| 281 | R | R | R | $ | F | L | H | H | L | I | A | E | I | H | T | A | | | -NH₂ |
| 282 | R | R | R | $ | F | L | H | H | L | I | A | E | I | H | T | A | | | -NH₂ |
| 283 | R | R | R | $ | F | L | H | H | L | I | A | E | I | H | T | A | | | -NH₂ |
| 284 | R | R | R | $ | F | L | H | H | L | I | A | E | I | H | T | A | | | -NH₂ |
| 285 | R | R | R | $ | F | L | H | H | L | I | A | E | I | H | T | A | | | -NH₂ |
| 286 | R | R | R | $ | F | L | H | H | L | I | A | E | I | H | T | A | | | -NH₂ |
| 287 | R | R | R | $ | F | L | H | H | L | I | A | E | I | H | T | A | | | -NH₂ |
| 288 | R | R | R | $ | F | L | H | H | L | I | A | E | I | H | T | A | | | -NH₂ |
| 289 | R | R | R | $ | F | L | H | H | L | I | A | E | I | H | T | A | | | -NH₂ |
| 290 | R | R | R | $ | F | $/ | H | H | L | I | A | E | I | H | T | A | | | -NH₂ |
| 291 | R | R | R | $ | F | L | $/ | H | L | I | A | E | I | H | T | A | | | -NH₂ |
| 292 | R | R | R | $ | F | L | Aib | H | L | I | A | E | I | H | T | A | | | -NH₂ |
| 293 | R | R | R | $ | F | L | H | $/ | L | I | A | E | I | H | T | A | | | -NH₂ |
| 294 | R | R | R | $ | F | L | H | Aib | L | I | A | E | I | H | T | A | | | -NH₂ |
| 295 | R | R | R | $ | F | L | H | H | $/ | I | A | E | I | H | T | A | | | -NH₂ |
| 296 | R | R | R | $ | F | L | H | H | L | $/ | A | E | I | H | T | A | | | -NH₂ |
| 297 | R | R | R | $ | F | L | H | H | L | I | $/ | E | I | H | T | A | | | -NH₂ |
| 298 | R | R | R | $ | F | L | H | H | L | I | Aib | E | I | H | T | A | | | -NH₂ |
| 299 | R | R | R | $ | F | L | H | H | L | I | A | E | $/ | H | T | A | | | -NH₂ |
| 300 | R | R | R | $ | F | L | H | H | L | I | A | E | I | $/ | T | A | | | -NH₂ |
| 301 | R | R | R | $ | F | L | H | H | L | I | $/ | E | I | H | T | A | | | -NH₂ |
| 302 | R | R | R | $ | F | L | H | H | L | $/ | A | E | I | H | T | A | | | -NH₂ |
| 303 | R | R | R | $ | F | L | H | H | L | $/ | A | E | I | H | T | A | | | -NH₂ |
| 304 | R | R | R | $ | F | L | H | H | L | I | $ | E | I | H | T | A | | | -NH₂ |
| 305 | R | R | R | $ | F | L | H | H | L | I | Aib | E | I | H | T | A | | | -NH₂ |
| 306 | R | R | R | $ | W | L | R | K | K | L | Q | D | V | H | N | F | | | -NH₂ |
| 307 | R | R | R | $ | F | L | R | K | K | L | Q | D | V | H | N | F | | | -NH₂ |
| 308 | R | R | R | $ | W | L | A | H | L | L | A | E | I | H | T | A | | | -NH₂ |
| 309 | R | R | R | F | W | $ | A | H | L | $ | A | E | I | H | T | A | | | -NH₂ |
| 310 | R | R | R | $ | W | L | H | A | L | L | A | E | I | H | T | A | | | -NH₂ |
| 311 | R | R | R | F | W | $ | H | A | L | $ | A | E | I | H | T | A | | | -NH₂ |
| 312 | R | R | R | $ | W | L | H | K | L | L | A | E | I | H | T | A | | | -NH₂ |
| 313 | R | R | R | F | W | $ | H | K | L | $ | A | E | I | H | T | A | | | -NH₂ |
| 314 | R | R | R | $ | W | L | H | H | L | L | A | E | I | H | T | A | | | -NH₂ |
| 315 | R | R | R | F | W | $ | H | H | L | $ | A | E | I | H | T | A | | | -NH₂ |
| 316 | R | R | R | $ | W | L | H | H | L | L | A | E | I | H | T | A | | | -NH₂ |
| 317 | R | R | R | F | W | $ | H | H | L | $ | A | E | I | H | T | A | | | -NH₂ |
| 318 | R | R | R | $ | W | L | H | H | L | L | A | E | I | H | T | A | | | -NH₂ |
| 319 | R | R | R | F | W | $ | H | H | L | $ | A | E | I | H | T | A | | | -NH₂ |
| 320 | R | R | R | $ | W | L | A | H | L | L | A | E | I | H | T | A | | | -NH₂ |
| 321 | R | R | R | F | W | $ | A | H | L | $ | A | E | I | H | T | A | | | -NH₂ |
| 322 | R | R | R | $ | W | L | A | H | L | L | A | E | I | H | T | A | | | -NH₂ |
| 323 | R | R | R | $ | W | L | R | $ | L | L | Q | D | V | H | N | F | | | -NH₂ |
| 324 | R | R | R | E | W | $ | R | K | L | $ | Q | D | V | H | N | F | | | -NH₂ |
| 325 | R | R | R | E | W | $ | R | K | L | $ | Q | D | V | H | N | F | | | -NH₂ |
| 326 | R | R | R | $ | W | L | R | $ | L | L | Q | D | V | H | N | F | | | -NH₂ |
| 327 | R | R | R | E | W | $ | R | K | L | $ | Q | D | V | H | N | F | | | -NH₂ |
| 328 | R | R | R | $ | W | L | R | $ | L | L | Q | D | V | H | N | F | | | -NH₂ |
| 329 | R | R | R | E | W | $ | R | K | L | $ | Q | D | V | H | N | F | | | -NH₂ |
| 330 | R | R | R | $ | W | L | R | $ | L | L | Q | D | V | H | N | F | | | -NH₂ |
| 331 | R | R | R | E | W | $ | R | K | L | $ | Q | D | V | H | N | F | | | -NH₂ |
| 332 | R | R | R | $ | W | L | R | $ | L | L | Q | D | V | H | N | F | | | -NH₂ |
| 333 | R | R | R | $ | W | L | R | $ | L | L | Q | D | V | H | N | F | | | -NH₂ |
| 334 | R | R | R | E | W | $ | R | K | L | $ | Q | D | V | H | N | F | | | -NH₂ |
| 335 | R | R | R | $ | W | L | R | Aib | K | L | Q | D | V | H | N | F | | | -NH₂ |
| 336 | R | R | R | $ | W | L | R | $ | K | L | Q | D | V | H | N | F | | | -NH₂ |
| 337 | R | R | R | E | W | $ | R | K | K | $ | Q | D | V | H | N | F | | | -NH₂ |
| 338 | R | R | R | E | W | L | R | $ | K | L | Q | $ | V | H | N | F | | | -NH₂ |
| 339 | R | R | R | E | W | $ | R | K | K | $ | Q | D | V | H | N | F | | | -NH₂ |
| 340 | R | R | R | $ | W | L | R | $ | L | L | Q | D | V | H | N | F | | | -NH₂ |
| 341 | R | R | R | E | W | $ | R | K | L | $ | Q | D | V | H | N | F | | | -NH₂ |
| 342 | R | R | R | $ | W | L | R | Aib | K | L | Q | D | V | H | N | F | | | -NH₂ |
| 343 | R | R | R | $ | W | L | R | $ | L | L | Q | D | V | H | N | F | | | -NH₂ |
| 344 | R | R | R | E | W | $ | R | K | L | $ | Q | D | V | H | N | F | | | -NH₂ |
| 345 | R | R | R | $ | W | L | R | Aib | K | L | Q | D | V | H | N | F | | | -NH₂ |
| 346 | R | R | R | $ | W | L | R | Aib | K | L | Q | D | V | H | N | F | | | -NH₂ |
| 347 | R | R | R | F | W | $ | A | H | L | $ | A | E | I | H | T | A | | | -NH₂ |
| 348 | R | R | R | $ | W | L | R | Aib | K | L | Q | D | V | | | | | | -NH₂ |
| 349 | R | R | R | $ | W | L | R | Aib | K | L | Q | D | V | H | E | F | | | -NH₂ |
| 350 | R | R | R | E | W | $ | R | K | L | $ | Q | D | V | H | E | F | | | -NH₂ |
| 351 | R | R | R | $ | W | L | R | Aib | K | L | Q | D | V | H | E | F | | | -NH₂ |
| 352 | R | R | R | E | W | $ | R | K | L | $ | Q | D | V | H | E | F | | | -NH₂ |
| 353 | R | R | R | $ | W | L | R | Aib | K | L | Q | D | V | H | E | F | | | -NH₂ |
| 354 | R | R | R | E | W | $ | R | K | L | $ | Q | D | V | H | E | F | | | -NH₂ |
| 355 | R | R | R | $ | W | L | R | Aib | K | L | Q | D | V | H | E | F | | | -NH₂ |
| 356 | R | R | R | E | W | $ | R | K | L | $ | Q | D | V | H | E | F | | | -NH₂ |
| 357 | R | R | R | $ | W | L | R | K | K | L | Q | D | V | H | N | F | | | -NH₂ |
| 358 | R | R | R | $ | W | L | R | K | | L | Q | D | V | H | N | F | | | -NH₂ |
| 359 | R | R | R | $ | W | L | R | Aib | K | L | Q | D | V | H | N | F | | | -NH₂ |
| 360 | R | R | R | $ | W | L | R | Aib | L | L | Q | D | V | H | N | F | | | -NH₂ |
| 361 | R | R | R | $ | W | L | R | Cit | L | L | Q | D | V | H | N | F | | | -NH₂ |
| 362 | R | R | R | E | W | L | R | K | K | L | Q | D | V | H | N | F | | | -NH₂ |
| 363 | R | R | R | $ | W | L | R | Aib | K | L | Q | D | V | H | N | F | G | Pra | -NH₂ |
| 364 | R | R | R | $ | W | L | R | AmO | L | L | Q | D | V | H | N | F | | | -NH₂ |
| 365 | R | R | R | F | W | $ | H | H | L | $ | A | E | I | H | T | A | | | -NH₂ |
| 366 | R | R | R | $ | W | L | R | $ | K | L | Q | D | V | H | N | F | | | -NH₂ |
| 367 | R | R | R | $ | W | L | H | H | L | I | A | E | I | H | T | A | | | -NH₂ |
| 368 | R | R | R | E | W | $ | R | K | K | $ | Q | D | V | H | N | F | | | -NH₂ |
| 369 | R | R | R | F | W | $ | H | H | L | $ | A | E | I | H | T | A | | | -NH₂ |
| 370 | R | R | R | $ | F | L | A | H | L | L | Aib | E | I | H | T | A | | | -NH₂ |
| 371 | R | R | R | $ | F | L | A | H | L | L | Aib | E | I | H | T | A | | | -NH₂ |
| 372 | R | R | R | $ | F | L | A | H | L | L | Aib | E | I | H | T | A | | | -NH₂ |
| 373 | R | R | R | $ | F | L | A | H | L | L | Aib | E | I | H | T | A | | | -NH₂ |
| 374 | R | R | R | $ | F | L | A | H | L | L | Aib | E | I | H | T | A | | | -NH₂ |
| 375 | R | R | R | $ | F | L | A | H | L | L | Aib | E | I | H | T | A | | | -NH₂ |
| 376 | R | R | R | $ | W | L | A | H | L | L | Aib | E | I | H | T | A | | | -NH₂ |
| 377 | R | R | R | $ | W | L | A | H | L | L | Aib | E | I | H | T | A | | | -NH₂ |
| 378 | R | R | R | $ | W | L | A | H | L | L | Aib | E | I | H | T | A | | | -NH₂ |
| 379 | R | R | R | $ | W | L | A | H | L | L | Aib | E | I | H | T | A | | | -NH₂ |
| 380 | R | R | R | $ | W | L | A | H | L | L | Aib | E | I | H | T | A | | | -NH₂ |
| 381 | R | R | R | $ | W | L | A | H | L | L | Aib | E | I | H | T | A | | | -NH₂ |
| 382 | E | R | V | $ | W | L | R | K | K | L | Q | D | V | H | E | F | | | -NH₂ |
| 383 | E | R | V | $ | W | L | R | K | K | L | Q | D | V | H | E | F | | | -NH₂ |
| 384 | R | R | R | $ | F | L | H | H | L | I | A | E | I | H | T | A | | | -NH₂ |
| 385 | R | R | R | $ | W | L | R | K | K | L | Q | D | V | H | E | F | | | -NH₂ |
| 386 | R | R | R | $ | W | L | R | K | K | L | Q | D | V | H | E | F | | | -NH₂ |
| 387 | R | R | V | $ | W | L | R | K | K | L | Q | D | V | H | E | F | | | -NH₂ |
| 388 | E | R | V | $ | W | L | R | Aib | K | L | Q | D | V | H | E | F | | | -NH₂ |
| 388 | E | R | V | $ | W | L | R | K | K | L | Q | D | V | H | E | F | | | -NH₂ |
| 389 | R | R | R | $ | W | L | R | Aib | K | L | Q | D | V | H | E | F | | | -NH₂ |
| 389 | R | R | R | $ | W | L | R | K | K | L | Q | D | V | H | E | F | | | -NH₂ |
| 390 | R | R | R | $ | W | L | R | Aib | K | L | Q | D | V | H | E | F | | | -NH₂ |
| 390 | R | R | R | $ | W | L | R | K | K | L | Q | D | V | H | E | F | | | -NH₂ |
| 391 | R | R | R | $ | F | L | H | H | L | I | A | E | I | H | T | A | | | -NH₂ |
| 391 | R | R | R | $ | F | L | H | K | L | I | A | E | I | H | T | A | | | -NH₂ |
| 392 | R | R | R | $ | W | L | R | K | K | L | Q | D | V | H | E | F | | | -NH₂ |
| 393 | R | R | R | $ | W | L | R | K | K | L | Q | D | V | H | E | F | | | -NH₂ |
| 394 | E | R | V | $r8 | W | L | R | K | K | L | Q | D | V | H | E | F | | | -NH₂ |
| 395 | R | R | R | $r8 | F | L | H | H | L | O | A | E | I | H | T | A | | | -NH₂ |
| 396 | R | R | R | $r8 | W | L | R | K | K | L | Q | D | V | H | E | F | | | -NH₂ |
| 397 | R | R | R | $r8 | W | L | R | K | K | L | Q | D | V | H | E | F | | | -NH₂ |
| 398 | R | R | V | $r8 | W | L | R | K | K | L | Q | D | V | H | E | F | | | -NH₂ |
| 399 | R | R | R | $ | W | L | H | K | L | L | Aib | E | I | H | T | A | | | -NH₂ |
| 400 | R | R | R | $ | W | L | H | K | L | L | Aib | E | I | H | T | A | | | -NH₂ |
| 400 | R | R | R | $ | W | L | H | K | L | L | Aib | E | I | H | T | A | | | -NH₂ |
| 400 | R | R | R | $ | W | L | H | K | L | I | Aib | E | I | H | T | A | | | -NH₂ |
| 401 | R | R | R | $r8 | W | L | H | H | L | I | A | E | I | H | T | A | | | -NH₂ |
| 402 | R | R | R | $ | W | L | H | H | L | I | A | E | I | H | T | A | | | -NH₂ |
| 403 | R | R | R | $ | F | L | H | H | L | L | A | E | I | H | T | A | | | -NH₂ |
| 404 | R | R | R | $ | F | L | H | H | L | L | Aib | E | I | H | T | A | | | -NH₂ |
| 405 | R | R | R | $ | F | L | A | H | L | L | Aib | E | I | H | T | A | | | -NH₂ |
| 406 | R | R | R | $ | W | L | H | H | L | L | Aib | E | I | H | T | A | | | -NH₂ |
| 407 | R | R | R | $ | W | L | A | H | L | L | Aib | E | I | H | T | A | | | -NH₂ |
| 408 | R | R | R | $ | F | L | H | H | L | L | Aib | E | I | H | T | A | | | -NH₂ |
| 409 | R | R | R | F | W | $ | H | K | L | $ | A | E | I | H | T | A | | | -NH₂ |
| 410 | $ | R | R | F | W | $ | H | K | L | $ | A | E | I | H | T | A | | | -NH₂ |
| 411 | R | R | R | F | W | $ | H | K | L | $ | A | E | I | H | T | A | | | -NH₂ |
| 412 | R | R | R | $ | W | L | H | K | L | L | Aib | E | I | H | T | A | | | -NH₂ |
| 413 | R | R | R | F | W | $ | H | K | L | $ | A | E | I | H | T | A | | | -NH₂ |
| 413 | R | R | R | F | W | $ | H | K | L | $ | A | E | I | H | T | A | | | -NH₂ |
| 413 | R | R | R | F | W | $ | H | K | L | $ | A | E | I | H | T | A | | | -NH₂ |
| 414 | R | R | R | F | W | $ | H | K | L | $ | A | E | I | H | T | A | | | -NH₂ |
| 414 | R | R | R | F | W | $ | H | K | L | $ | A | E | I | H | T | A | | | -NH₂ |
| 414 | R | R | R | F | W | $ | H | K | L | $ | A | E | I | H | T | A | | | -NH₂ |
| 415 | R | R | R | $ | W | L | H | K | L | L | Aib | E | I | H | T | A | | | -NH₂ |
| 415 | R | R | R | $ | W | L | H | K | L | L | Aib | E | I | H | T | A | | | -NH₂ |
| 415 | R | R | R | $ | W | L | H | K | L | L | Aib | E | I | H | T | A | | | -NH₂ |
| 416 | R | R | R | $ | W | L | R | Aib | L | L | Q | D | V | H | E | F | | | -NH₂ |
| 417 | R | R | R | $ | W | L | H | H | L | L | Q | D | V | H | E | F | | | -NH₂ |
| 418 | R | R | R | $ | W | L | H | K | L | L | Q | D | V | H | E | F | | | -NH₂ |
| 419 | R | R | R | $ | W | L | R | H | L | L | Q | D | V | H | E | F | | | -NH₂ |
| 420 | R | R | R | $ | W | L | Aib | K | L | L | Q | D | V | H | E | F | | | -NH₂ |
| 421 | R | R | R | $ | W | L | H | Aib | L | L | Q | D | V | H | E | F | | | -NH₂ |
| 422 | R | R | R | $ | W | L | A | H | L | L | Q | D | V | H | E | F | | | -NH₂ |
| 423 | R | R | R | $ | W | L | Aib | H | L | L | Q | D | V | H | E | F | | | -NH₂ |
| 424 | R | R | R | $ | W | L | r | K | L | L | Q | D | V | H | E | F | | | -NH₂ |
| 425 | R | R | R | $ | W | L | R | k | L | L | Q | D | V | H | E | F | | | -NH₂ |
| 426 | R | R | R | $ | W | L | r | k | L | L | Q | D | V | H | E | F | | | -NH₂ |
| 427 | R | R | R | $ | W | L | R | Aib | L | L | Q | D | V | H | E | F | | | -NH₂ |
| 428 | R | R | R | $ | W | L | R | Aib | L | L | Q | D | V | H | E | F | | | -NH₂ |
| 429 | R | R | R | $r8 | W | L | R | Aib | L | L | Q | D | V | H | E | F | | | -NH₂ |
| 430 | R | R | R | $r8 | W | L | R | Aib | K | L | Q | D | V | H | E | F | | | -NH₂ |
| 431 | R | R | R | $ | W | L | R | Aib | K | L | Q | D | V | H | E | F | | | -NH₂ |
| 432 | R | R | R | $ | W | L | R | K | K | L | Q | D | V | H | N | Y | | | -NH₂ |
| 433 | R | R | R | $ | W | L | R | K | K | L | Q | D | V | H | N | Y | | | -NH₂ |
| 434 | R | R | R | $ | W | L | R | K | K | L | Q | D | V | H | E | F | | | -NH₂ |
| 435 | R | R | R | $ | F | L | H | H | L | I | A | E | I | H | T | A | | | -NH₂ |
| 435 | R | R | R | $ | F | L | H | K | L | I | A | E | I | H | T | A | | | -NH₂ |
| 436 | R | R | V | $ | W | L | R | Aib | K | L | Q | E | V | H | E | F | | | -NH₂ |
| 436 | R | R | V | $ | W | L | R | K | K | L | Q | E | V | H | E | F | | | -NH₂ |
| 437 | R | R | R | $ | W | L | R | K | K | L | Q | E | V | H | E | F | | | -NH₂ |
| 438 | R | R | R | $ | W | L | R | $ | K | L | Q | E | V | H | E | F | | | -NH₂ |
| 439 | R | R | R | $ | W | L | R | $ | K | L | Q | E | V | H | E | F | | | -NH₂ |
| 440 | R | R | R | $ | W | $ | R | K | L | $ | Q | E | V | H | E | F | | | -NH₂ |
| 441 | R | R | R | $ | W | $ | R | K | K | $ | Q | E | V | H | E | F | | | -NH₂ |
| 442 | R | R | R | $ | W | $ | R | K | K | $ | Q | E | V | H | E | F | | | -NH₂ |
| 443 | R | R | R | $ | W | $ | R | K | K | $ | Q | E | V | H | E | F | | | -NH₂ |
| 444 | R | R | R | $ | W | L | R | K | K | L | Q | E | V | | | | | | -NH₂ |
| 445 | R | R | R | E | W | L | R | $ | K | L | Q | E | V | H | E | F | | | -NH₂ |

Table 2
Linear Peptidomimetic

| SEQ | | | | | | | | | | | | | | | | | | | | |
|---|---|---|---|---|---|---|---|---|---|---|---|---|---|---|---|---|---|---|---|---|
| ID | SP# | 0 | 1 | 2 | 3 | 4 | 5 | 6 | 7 | 8 | 9 | 10 | 11 | 12 | 13 | 14 | 15 | 16 | 17 | 18 |
| 6 | PTH | H- | S | V | S | E | I | Q | L | M | H | N | L | G | K | H | L | N | S | M |
| 7 | PTHrP | H- | A | V | S | E | H | Q | L | L | H | D | K | G | K | S | I | Q | D | L |

| SEQ | | | | | | | | | | | | | | | | | | | |
|---|---|---|---|---|---|---|---|---|---|---|---|---|---|---|---|---|---|---|---|
| ID | 19 | 20 | 21 | 22 | 23 | 24 | 25 | 26 | 27 | 28 | 29 | 30 | 31 | 32 | 33 | 34 | 35 | 36 | 37 |
| 6 | E | R | V | E | W | L | R | K | K | L | Q | D | V | H | N | F | | | -OH |
| 7 | R | R | R | F | F | L | H | H | L | I | A | E | I | H | T | A | E | Y | -NH₂ |

Table 2a
Linear Peptidomimetic

| SEQ | | | | | | | | | | | | | | | | | | | | |
|---|---|---|---|---|---|---|---|---|---|---|---|---|---|---|---|---|---|---|---|---|
| ID | SP# | 0 | 1 | 2 | 3 | 4 | 5 | 6 | 7 | 8 | 9 | 10 | 11 | 12 | 13 | 14 | 15 | 16 | 17 | 18 |
| 446 | LP96 | H- | | | | | | | L | L | H | N | L | G | K | H | L | N | S | L |
| 447 | LP1 | H- | | | Deg | E | I | Q | L | L | H | N | L | G | K | H | L | N | S | L |
| 448 | LP2 | H- | Deg | V | Deg | E | I | Q | L | L | H | N | L | G | K | H | L | N | S | L |
| 449 | LP3 | PhAc- | S | V | Deg | E | I | Q | L | L | H | N | L | G | K | H | L | N | S | L |
| 450 | LP4 | H- | S | W | Deg | E | I | Q | L | L | H | N | L | G | K | H | L | N | S | L |
| 451 | LP5 | H- | S | R | Deg | E | I | Q | L | L | H | N | L | G | K | H | L | N | S | L |
| 452 | LP6 | H- | S | w | Deg | E | I | Q | L | L | H | N | L | G | K | H | L | N | S | L |
| 453 | LP7 | H- | S | r | Deg | E | I | Q | L | L | H | N | L | G | K | H | L | N | S | L |
| 454 | LP8 | H- | S | F4COOH | Deg | E | I | Q | L | L | H | N | L | G | K | H | L | N | S | L |
| 455 | LP9 | H- | S | Bip | Deg | E | I | Q | L | L | H | N | L | G | K | H | L | N | S | L |
| 456 | LP10 | H- | S | F4NH2 | Deg | E | I | Q | L | L | H | N | L | G | K | H | L | N | S | L |
| 457 | LP11 | H- | 1Nal | V | S | E | I | Q | L | L | H | N | L | G | K | H | L | N | S | $ |
| 458 | LP12 | H- | 2Nal | V | S | E | I | Q | L | L | H | N | L | G | K | H | L | N | S | L |
| 459 | LP13 | 2NaAc- | | | | E | I | Q | L | L | H | N | L | G | K | H | L | N | S | L |
| 460 | LP14 | 2NaAc- | | | Deg | E | I | Q | L | L | H | N | L | G | K | H | L | N | S | L |
| 461 | LP15 | H- | | | E | E | I | Q | L | L | H | N | L | G | K | H | L | N | S | L |
| 462 | LP16 | H- | | | K | E | I | Q | L | L | H | N | L | G | K | H | L | N | S | L |
| 463 | LP17 | H- | | | F | E | I | Q | L | L | H | N | L | G | K | H | L | N | S | L |
| 464 | LP18 | H- | | | S | Q | I | Q | L | L | H | N | L | G | K | H | L | N | S | L |
| 465 | LP19 | H- | | | S | W | I | Q | L | L | H | N | L | G | K | H | L | N | S | L |
| 466 | LP20 | H- | | | S | F | I | Q | L | L | H | N | L | G | K | H | L | N | S | L |
| 467 | LP21 | H- | | | S | H | I | Q | L | L | H | N | L | G | K | H | L | N | S | L |
| 468 | LP22 | H- | | | S | E | K | Q | L | L | H | N | L | G | K | H | L | N | S | L |
| 469 | LP23 | H- | | | S | E | E | Q | L | L | H | N | L | G | K | H | L | N | S | L |
| 470 | LP24 | H- | | | S | E | F | Q | L | L | H | N | L | G | K | H | L | N | S | L |
| 471 | LP25 | H- | | | S | E | I | K | L | L | H | N | L | G | K | H | L | N | S | L |
| 472 | LP6 | H- | | | S | E | I | E | L | L | H | N | L | G | K | H | L | N | S | L |
| 473 | LP27 | H- | | | S | E | I | F | L | L | H | N | L | G | K | H | L | N | S | L |
| 474 | LP28 | H- | | | S | E | I | Q | K | L | H | N | L | G | K | H | L | N | S | L |
| 475 | LP29 | H- | | | S | E | I | Q | E | L | H | N | L | G | K | H | L | N | S | L |
| 476 | LP30 | H- | | | S | E | I | Q | A | L | H | N | L | G | K | H | L | N | S | L |
| 477 | LP31 | H- | | | S | E | I | Q | F | L | H | N | L | G | K | H | L | N | S | L |
| 478 | LP32 | H- | | | A | L | A | D | D | L | H | N | L | G | K | H | L | N | S | L |
| 479 | LP33 | H- | | | | | | | F | L | H | N | L | G | K | H | L | N | S | L |
| 480 | LP34 | H- | | | | | | | L | L | H | N | L | w | K | H | L | N | S | L |
| 481 | LP35 | H- | | | | | | | L | L | H | N | L | G | K | H | L | N | S | L |
| 482 | LP36 | H- | | | | | | | L | L | H | N | L | G | K | H | L | N | S | L |
| 483 | LP37 | H- | | | | | | | L | L | H | N | L | G | K | H | L | N | S | L |
| 484 | LP38 | H- | | | | | | | L | L | H | N | L | G | K | H | L | N | S | L |
| 485 | LP39 | H- | | | | | | | L | L | H | N | L | G | K | H | L | N | S | L |
| 486 | LP40 | H- | | | | | | | L | L | H | N | L | G | K | H | L | N | S | L |
| 487 | LP41 | H- | | | | | | | L | L | H | N | L | G | K | H | L | N | S | L |
| 488 | LP42 | H- | | | | | | | L | L | H | N | L | G | K | H | L | N | S | L |
| 489 | LP43 | H- | | | | | | | L | L | H | N | L | G | K | H | L | N | S | L |
| 490 | LP44 | H- | | | | | | | L | L | H | N | L | G | K | H | L | N | $ | L |
| 491 | LP45 | H- | | | | | | | L | L | H | N | L | G | K | H | L | N | S | L |
| 492 | LP46 | H- | | | | | | | L | L | H | N | L | G | K | H | L | N | S | L |
| 493 | LP47 | H- | | | | | | | L | L | H | N | L | G | K | H | L | N | S | L |
| 494 | LP48 | H- | | | | | | | L | L | H | N | L | G | K | H | L | N | S | L |
| 495 | LP49 | H- | | | | | | | L | L | H | N | L | G | K | H | L | N | S | L |
| 496 | LP50 | H- | | | | | | | L | L | H | N | L | G | K | H | L | N | S | L |
| 497 | LP51 | H- | | | | | | | L | L | H | N | L | G | K | H | L | N | S | L |
| 498 | LP52 | H- | | | | | | | L | L | H | N | L | G | K | H | L | N | S | L |
| 499 | LP53 | H- | | | | | | | L | L | H | N | L | G | K | H | L | N | S | L |
| 500 | LP54 | H- | | | | | | | L | L | H | N | L | G | K | H | L | N | S | L |
| 501 | LP55 | H- | | | | | | | L | L | H | N | L | G | K | H | L | N | S | L |
| 502 | LP56 | H- | | | | | | | L | L | H | N | L | G | K | H | L | N | S | L |
| 503 | LP57 | H- | | | | | | | L | L | H | N | L | G | K | H | L | N | S | L |
| 504 | LP58 | H- | | | | | | | L | L | H | N | L | G | K | H | L | N | S | L |
| 505 | LP59 | H- | | | | | | | L | L | H | N | L | G | K | H | L | N | S | L |
| 506 | LP60 | H- | | | | | | | L | L | H | N | L | G | K | H | L | N | S | L |
| 507 | LP61 | H- | | | | | | | L | L | H | N | L | G | K | H | L | N | S | L |
| 508 | LP62 | H- | | | | | | | L | L | H | N | L | G | K | H | L | N | S | L |
| 509 | LP63 | H- | | | | | | | L | L | H | N | L | G | K | H | L | N | S | L |
| 510 | LP64 | H- | | | | | | | L | L | H | N | L | G | K | H | L | N | S | L |
| 511 | LP65 | H- | | | | | | | L | L | H | N | L | G | K | H | L | N | S | L |
| 512 | LP66 | H- | | | | | | | L | L | H | Q | L | G | K | H | L | N | S | L |
| 513 | LP67 | H- | | | | | | | L | L | H | N | hR | G | K | H | L | N | S | L |
| 514 | LP68 | H- | | | | | | | L | L | H | N | L | A | K | H | L | N | S | L |
| 515 | LP69 | H- | | | | | | | L | L | H | N | L | G | K | W | L | N | S | L |
| 516 | LP70 | H- | | | | | | | L | L | H | Q | hR | A | K | W | L | N | S | L |
| 517 | LP71 | H- | | | | | | | L | M | H | Q | hR | A | K | W | I | Q | D | L |
| 518 | LP72 | H- | | | | | | | L | L | H | Q | hR | A | K | W | I | Q | D | L |
| 519 | LP73 | H- | | | | | | | L | L | H | Q | hR | w | K | W | I | Q | D | L |
| 520 | LP74 | H- | | | | | | | L | L | H | Q | hR | A | K | W | I | Q | D | L |
| 521 | LP75 | H- | | | | | | | L | Nle | H | Q | hR | A | S | W | L | N | S | Nle |
| 522 | LP76 | H- | | | | | | | L | Nle | H | Q | hR | A | S | W | L | N | S | Nle |

| SEQ | | | | | | | | | | | | | | | | | | | |
|---|---|---|---|---|---|---|---|---|---|---|---|---|---|---|---|---|---|---|---|
| ID | 19 | 20 | 21 | 22 | 23 | 24 | 25 | 26 | 27 | 28 | 29 | 30 | 31 | 32 | 33 | 34 | 35 | 36 | 37 |
| 446 | E | R | V | E | W | L | R | K | K | L | $ | D | V | H | $ | F | | | -NH₂ |
| 447 | E | R | V | E | W | L | R | K | K | L | Q | D | V | H | N | F | | | -NH₂ |
| 448 | E | R | V | E | W | L | R | K | K | L | Q | D | V | H | N | F | | | -NH₂ |
| 449 | E | R | V | E | W | L | R | K | K | L | Q | D | V | H | N | F | | | -NH₂ |
| 450 | E | R | V | E | W | L | R | K | K | L | Q | D | V | H | N | F | | | -NH₂ |
| 451 | E | R | V | E | W | L | R | K | K | L | Q | D | V | H | N | F | | | -NH₂ |
| 452 | E | R | V | E | W | L | R | K | K | L | Q | D | V | H | N | F | | | -NH₂ |
| 453 | E | R | V | E | W | L | R | K | K | L | Q | D | V | H | N | F | | | -NH₂ |
| 454 | E | R | V | E | W | L | R | K | K | L | Q | D | V | H | N | F | | | -NH₂ |
| 455 | E | R | V | E | W | L | R | K | K | L | Q | D | V | H | N | F | | | -NH₂ |
| 456 | $ | R | V | E | W | L | R | K | K | L | Q | D | V | H | N | F | | | -NH₂ |
| 457 | E | R | V | E | W | L | R | K | K | L | Q | D | V | H | N | F | | | -NH₂ |
| 458 | E | R | V | E | W | L | R | K | K | L | Q | D | V | H | N | F | | | -NH₂ |
| 459 | E | R | V | E | W | L | R | K | K | L | Q | D | V | H | N | F | | | -NH₂ |
| 460 | E | R | V | E | W | L | R | K | K | L | Q | D | V | H | N | F | | | -NH₂ |
| 461 | E | R | V | E | W | L | R | K | K | L | Q | D | V | H | N | F | | | -NH₂ |
| 462 | E | R | V | E | W | L | R | K | K | L | Q | D | V | H | N | F | | | -NH₂ |
| 463 | E | R | V | E | W | L | R | K | K | L | Q | D | V | H | N | F | | | -NH₂ |
| 464 | E | R | V | E | W | L | R | K | K | L | Q | D | V | H | N | F | | | -NH₂ |
| 465 | E | R | V | E | W | L | R | K | K | L | Q | D | V | H | N | F | | | -NH₂ |
| 466 | E | R | V | E | W | L | R | K | K | L | Q | D | V | H | N | F | | | -NH₂ |
| 467 | E | R | V | E | W | L | R | K | K | L | Q | D | V | H | N | F | | | -NH₂ |
| 468 | E | R | V | E | W | L | R | K | K | L | Q | D | V | H | N | F | | | -NH₂ |
| 469 | E | R | V | E | W | L | R | K | K | L | Q | D | V | H | N | F | | | -NH₂ |
| 470 | E | R | V | E | W | L | R | K | K | L | Q | D | V | H | N | F | | | -NH₂ |
| 471 | E | R | V | E | W | L | R | K | K | L | Q | D | V | H | N | F | | | -NH₂ |
| 472 | E | R | V | E | W | L | R | K | K | L | Q | D | V | H | N | F | | | -NH₂ |
| 473 | E | R | V | E | W | L | R | K | K | L | Q | D | V | H | N | F | | | -NH₂ |
| 474 | E | R | V | E | W | L | R | K | K | L | Q | D | V | H | N | F | | | -NH₂ |
| 475 | E | R | V | E | W | L | R | K | K | L | Q | D | V | H | N | F | | | -NH₂ |
| 476 | E | R | V | E | W | L | R | K | K | L | Q | D | V | H | N | F | | | -NH₂ |
| 477 | E | R | V | E | W | L | R | K | K | L | Q | D | V | H | N | F | | | -NH₂ |
| 478 | E | R | V | E | W | L | R | K | K | L | Q | D | V | H | N | F | | | -NH₂ |
| 479 | E | R | V | E | W | L | R | K | K | L | Q | D | V | H | N | F | | | -NH₂ |
| 480 | E | R | V | E | W | L | R | K | K | L | Q | D | V | H | N | F | | | -NH₂ |
| 481 | E | R | R | E | W | L | R | K | K | L | Q | D | V | H | N | F | | | -NH₂ |
| 482 | E | R | K | E | W | L | R | K | K | L | Q | D | V | H | N | F | | | -NH₂ |
| 483 | E | R | V | E | 1Nal | L | R | K | K | L | Q | D | V | H | N | F | | | -NH₂ |
| 484 | E | R | V | E | 2Nal | L | R | K | K | L | Q | D | V | H | N | F | | | -NH₂ |
| 485 | E | R | V | E | 9- | L | R | K | K | L | Q | D | V | H | N | F | | | -NH₂ |
| | | | | | Aal | | | | | | | | | | | | | | |
| 486 | E | R | V | E | W | L | R | K | K | L | Q | D | V | H | N | F | | | -NH₂ |
| 487 | E | R | V | E | W | L | R | K | hF | L | Q | D | V | H | N | F | | | -NH₂ |
| 488 | E | R | V | E | W | L | R | K | R | L | Q | D | V | H | N | F | | | -NH₂ |
| 489 | E | R | V | E | W | L | R | K | Nle | L | Q | D | V | H | N | F | | | -NH₂ |
| 490 | E | R | V | E | W | L | R | K | Y | L | Q | D | V | H | N | F | | | -NH₂ |
| 491 | E | R | V | E | W | L | R | K | H | L | Q | D | V | H | N | F | | | -NH₂ |
| 492 | E | R | V | E | W | L | R | K | F | L | Q | D | V | H | N | F | | | -NH₂ |
| 493 | E | R | V | E | W | L | R | K | K | L | Q | R | V | H | N | F | | | -NH₂ |
| 494 | E | R | V | E | W | L | R | K | Y | L | Q | R | V | H | N | F | | | -NH₂ |
| 495 | E | R | V | E | W | L | R | K | L | L | Q | hF | V | H | N | F | | | -NH₂ |
| 496 | E | R | V | E | W | L | R | K | L | L | Q | R | V | H | N | F | | | -NH₂ |
| 497 | E | R | V | E | W | L | R | K | K | L | Q | L | V | H | N | F | | | -NH₂ |
| 498 | E | R | V | E | W | L | R | K | K | L | Q | D | Nle | H | N | F | | | -NH₂ |
| 499 | E | R | V | E | W | L | R | K | K | L | Q | D | T | H | N | F | | | -NH₂ |
| 500 | E | R | V | E | W | L | R | K | K | L | Q | D | S | H | N | F | | | -NH₂ |
| 501 | E | R | V | E | W | L | R | K | K | L | Q | D | V | W | N | F | | | -NH₂ |
| 502 | E | R | V | E | W | L | R | K | K | L | Q | D | V | R | N | F | | | -NH₂ |
| 503 | E | R | V | E | W | L | R | K | K | L | Q | D | V | F | N | F | | | -NH₂ |
| 504 | E | R | V | E | W | L | R | K | K | L | Q | D | V | Y | N | F | | | -NH₂ |
| 505 | E | R | V | E | W | L | R | K | K | L | Q | D | V | I | N | F | | | -NH₂ |
| 506 | E | R | V | E | W | L | R | K | K | L | Q | D | V | H | E | F | | | -NH₂ |
| 507 | E | R | V | E | W | L | H | K | K | L | Q | D | V | H | D | F | | | -NH₂ |
| 508 | E | R | V | E | W | L | R | K | K | L | Q | D | V | H | N | Y | | | -NH₂ |
| 509 | E | R | V | E | W | L | R | K | K | L | Q | D | V | H | N | R | | | -NH₂ |
| 510 | E | R | V | E | W | L | R | K | K | L | Q | D | V | H | N | 2Nal | | | -NH₂ |
| 511 | E | R | V | E | W | L | R | K | K | L | Q | D | V | H | N | hF | | | -NH₂ |
| 512 | E | R | V | E | W | L | R | K | K | L | Q | D | V | H | N | F | | | -NH₂ |
| 513 | E | R | V | E | W | L | R | K | K | L | Q | D | V | H | N | F | | | -NH₂ |
| 514 | E | R | V | E | W | L | R | K | K | L | Q | D | V | H | N | F | | | -NH₂ |
| 515 | E | R | V | E | W | L | R | K | K | L | Q | D | V | H | N | F | | | -NH₂ |
| 516 | E | R | V | E | W | L | R | K | K | L | Q | D | V | H | N | F | | | -NH₂ |
| 517 | R | R | R | F | F | L | H | H | H | I | A | E | I | H | T | A | E | Y | -NH₂ |
| 518 | R | R | R | F | W | L | H | H | H | I | A | E | I | H | T | A | E | Y | -NH₂ |
| 519 | R | R | R | F | W | L | H | H | H | I | A | E | I | H | T | A | E | Y | -NH₂ |
| 520 | R | R | R | F | F | L | H | H | H | I | A | E | I | H | T | A | E | Y | -NH₂ |
| 521 | S | R | Q | S | W | L | R | K | Q | L | Q | N | V | H | N | F | | | -NH₂ |
| 522 | R | R | Q | S | W | L | R | K | Q | L | Q | N | V | H | N | F | | | -NH₂ |

Table 2b
Linear Peptidomimetic

| SEQ | | | | | | | | | | | | | | | | | | | | |
|---|---|---|---|---|---|---|---|---|---|---|---|---|---|---|---|---|---|---|---|---|
| ID | SP# | 0 | 1 | 2 | 3 | 4 | 5 | 6 | 7 | 8 | 9 | 10 | 11 | 12 | 13 | 14 | 15 | 16 | 17 | 18 |
| 523 | LP77 | Ac- | | | | | | | L | L | H | Q | R | A | K | W | L | N | S | L |
| 524 | LP78 | Ac- | | | | | | | L | L | H | D | K | A | K | S | I | Q | D | L |
| 525 | LP79 | Ac- | | | | | | | L | L | H | Q | R | A | K | W | I | Q | D | L |
| 526 | LP80 | Ac- | | | | | | | L | L | H | D | K | A | K | S | I | Q | D | L |
| 527 | LP81 | Ac- | | | | | | | L | L | H | Q | R | A | K | W | L | N | S | L |
| 528 | LP82 | Ac- | | | | | | | L | L | H | Q | R | A | K | W | L | N | S | L |
| 529 | LP83 | Ac- | | | | | | | L | L | H | Q | R | A | K | W | I | Q | D | L |
| 530 | LP84 | Ac- | | | | | | | L | L | H | Q | R | A | K | W | L | N | S | L |
| 531 | LP85 | Ac- | | | | | | | L | L | H | Q | R | Aib | K | W | L | N | S | Aib |
| 532 | LP86 | Ac- | | | | | | | L | L | H | D | K | Aib | K | S | I | Q | D | Aib |
| 533 | LP87 | Ac- | | | | | | | L | L | H | Q | R | Aib | K | W | I | Q | D | Aib |
| 534 | LP88 | Ac- | | | | | | | L | L | H | D | K | Aib | K | S | I | Q | D | Aib |
| 535 | LP89 | Ac- | | | | | | | L | L | H | Q | R | Aib | K | W | L | N | S | Aib |
| 536 | LP90 | Ac- | | | | | | | L | L | H | Q | R | Aib | K | W | L | N | S | Aib |
| 537 | LP91 | Ac- | | | | | | | L | L | H | Q | R | Aib | K | W | I | Q | D | Aib |
| 538 | LP92 | Ac- | | | | | | | L | L | H | Q | R | Aib | K | W | L | N | S | Aib |
| 539 | LP93 | Ac- | | | | | | | L | L | H | Q | R | A | K | W | I | Q | D | L |
| 540 | LP94 | Ac- | | | | | | | L | L | H | Q | R | A | K | D- | I | Q | D | L |
| | | | | | | | | | | | | | | | | Trp | | | | |
| 541 | LP95 | Ac- | | | | | | | L | L | H | Q | R | Aib | K | W | I | Q | D | Aib |

| SEQ | | | | | | | | | | | | | | | | | | | |
|---|---|---|---|---|---|---|---|---|---|---|---|---|---|---|---|---|---|---|---|
| ID | 19 | 20 | 21 | 22 | 23 | 24 | 25 | 26 | 27 | 28 | 29 | 30 | 31 | 32 | 33 | 34 | 35 | 36 | 37 |
| 523 | E | R | V | E | W | L | R | K | K | L | Q | D | V | H | E | F | | | NH₂ |
| 524 | R | R | R | F | F | L | H | H | L | I | A | E | I | H | T | A | | | NH₂ |
| 525 | R | R | R | F | F | L | H | H | L | I | A | E | I | H | T | A | | | NH₂ |
| 526 | R | R | R | E | W | L | R | K | K | L | Q | D | V | H | E | F | | | NH₂ |
| 527 | R | R | R | E | W | L | R | K | K | L | Q | D | V | H | E | F | | | NH₂ |
| 528 | R | R | V | E | W | L | R | K | K | L | Q | D | V | H | E | F | | | NH₂ |
| 529 | R | R | R | F | W | L | R | K | K | L | Q | D | V | H | E | F | | | NH₂ |
| 530 | R | R | R | F | W | L | R | K | K | L | Q | D | V | H | E | F | | | NH₂ |
| 531 | E | R | V | Aib | W | L | R | Aib | K | L | Q | D | V | H | E | F | | | NH₂ |
| 532 | R | R | R | Aib | F | L | H | Aib | L | I | A | E | I | H | T | A | | | NH₂ |
| 533 | R | R | R | Aib | F | L | H | Aib | L | I | A | E | I | H | T | A | | | NH₂ |
| 534 | R | R | R | Aib | W | L | R | Aib | K | L | Q | D | V | H | E | F | | | NH₂ |
| 535 | R | R | R | Aib | W | L | R | Aib | K | L | Q | D | V | H | E | F | | | NH₂ |

| | | | | | | | | | | | | | | | | | |
|---|---|---|---|---|---|---|---|---|---|---|---|---|---|---|---|---|---|
| 536 | R | R | V | Aib | W | L | R | Aib | K | L | Q | D | V | H | E | F | NH₂ |

| | | | | | | | | | | | | | | | | | |
|---|---|---|---|---|---|---|---|---|---|---|---|---|---|---|---|---|---|
| 537 | R | R | R | Aib | W | L | R | Aib | K | L | Q | D | V | H | E | F | NH₂ |
| 538 | E | R | R | Aib | W | L | R | Aib | K | L | Q | D | V | H | E | F | NH₂ |
| 539 | R | R | R | E | W | L | R | K | K | L | Q | D | V | H | N | Y | NH₂ |
| 540 | R | R | R | E | W | L | R | K | K | L | Q | D | V | H | N | Y | NH₂ |
| 541 | R | R | R | Aib | W | L | R | Aib | L | L | Q | D | V | H | E | F | NH₂ |

Table 3
Exemplary Peptidomimetic Macrocycles

| SEQ | | | | | | | | | | | | | | | | | | | | |
|---|---|---|---|---|---|---|---|---|---|---|---|---|---|---|---|---|---|---|---|---|
| ID | SP# | 0 | 1 | 2 | 3 | 4 | 5 | 6 | 7 | 8 | 9 | 10 | 11 | 12 | 13 | 14 | 15 | 16 | 17 | 18 |
| 6 | PTH | H- | S | V | S | E | I | Q | L | M | H | N | L | G | K | H | L | N | S | M |
| 7 | PTHrP | H- | A | V | S | E | H | Q | L | L | H | D | K | G | K | S | I | Q | D | L |
| SEQ | | | | | | | | | | | | | | | | | | | | |
| ID | SP# | 19 | 20 | 21 | 22 | 23 | 24 | 25 | 26 | 27 | 28 | 29 | 30 | 31 | 32 | 33 | 34 | 35 | 36 | 37 |
| 6 | PTH | E | R | V | E | W | L | R | K | K | L | Q | D | V | H | N | F | | | -OH |
| 7 | PTHrP | R | R | R | F | F | L | H | H | L | I | A | E | I | H | T | A | E | Y | -NH₂ |

Table 3a
Exemplary Peptidomimetic Macrocycles

| SEQ | | | | | | | | | | | | | | | | | | | | |
|---|---|---|---|---|---|---|---|---|---|---|---|---|---|---|---|---|---|---|---|---|
| ID | SP# | 0 | 1 | 2 | 3 | 4 | 5 | 6 | 7 | 8 | 9 | 10 | 11 | 12 | 13 | 14 | 15 | 16 | 17 | 18 |
| 63 | 57 | H- | | | | | | | L | L | H | N | L | G | $ | H | L | N | $ | L |
| 120 | 114 | H- | | | | | | | L | L | H | Q | hR | A | K | W | I | Q | D | $ |
| 20 | 14 | H- | | | | | | | L | L | H | N | L | G | $ | H | L | N | $ | L |
| 21 | 15 | H- | | | | | | | L | L | H | N | L | $ | K | H | L | $ | S | L |
| 22 | 16 | H- | | | | | | | L | L | H | N | $ | G | K | H | $ | N | S | L |
| 23 | 17 | H- | | | | | | | L | L | H | $ | L | G | K | $ | L | N | S | L |
| 24 | 18 | H- | | | | | | | L | L | $ | N | L | G | $ | H | L | N | S | L |
| 25 | 19 | H- | | | | | | | L | L | H | N | L | $ | K | H | L | N | S | L |
| 46 | 40 | H- | | | | | | | L | L | H | N | L | G | K | H | $r8 | N | S | L |
| 48 | 42 | H- | | | | | | | L | L | H | N | L | G | K | $r8 | L | N | S | L |
| 64 | 58 | H- | | | | | | | L | L | H | N | L | A | $ | H | L | N | $ | L |
| 67 | 61 | H- | | | | | | | L | L | H | N | L | G | $ | H | L | N | $ | $ |
| 68 | 62 | H- | | | | | | | L | L | H | N | L | G | K | $ | L | N | S | $ |
| 72 | 66 | H- | | | | | | | L | L | H | Q | hR | A | $ | W | I | Q | $ | L |
| 73 | 67 | H- | | | | | | | L | L | H | Q | hR | A | K | $ | I | Q | D | $ |
| 78 | 72 | H- | | | | | | | L | L | H | Q | hR | A | K | $ | I | Q | D | $ |
| 49 | 43 | H- | | | | | | | L | L | H | N | L | G | K | $r8 | L | N | S | L |
| 77 | 71 | H- | | | | | | | L | L | H | D | K | G | K | $ | I | Q | D | $ |
| 85 | 79 | H- | | | | | | | L | L | H | Q | hR | A | K | $ | I | Q | D | $ |
| 89 | 83 | H- | | | | | | | L | L | H | Q | hR | A | K | $ | I | Q | D | $ |
| 86 | 80 | H- | | | | | | | L | L | H | Q | hR | A | K | $ | I | Q | D | $ |
| 74 | 68 | H- | | | | | | | L | L | H | Q | hR | A | K | $ | I | Q | D | $ |
| 123 | 117 | H- | | | | | | | L | L | H | Q | hR | A | K | $ | I | Q | D | St |
| 124 | 118 | H- | | | | | | | L | L | H | Q | hR | A | K | $r5 | I | Q | D | $ |
| 80 | 74 | H- | | | | | | | F | L | H | Q | hR | A | K | $ | L | N | S | $ |
| 79 | 73 | H- | | | | | | | L | L | H | Q | hR | A | K | $ | L | N | S | $ |
| 82 | 76 | H- | | | | | | | L | L | H | D | K | G | K | $ | I | Q | D | $ |
| SEQ | | | | | | | | | | | | | | | | | | | | |
| ID | SP# | 19 | 20 | 21 | 22 | 23 | 24 | 25 | 26 | 27 | 28 | 29 | 30 | 31 | 32 | 33 | 34 | 35 | 36 | 37 |
| 63 | 57 | E | R | V | E | W | L | R | $ | K | L | Q | $ | V | H | N | F | | | -NH₂ |
| 120 | 114 | R | R | R | $ | F | L | H | H | L | I | A | E | I | H | T | A | E | Y | -NH₂ |
| 20 | 14 | E | R | V | E | W | L | R | K | K | L | Q | D | V | H | N | F | | | -NH₂ |
| 21 | 15 | E | R | V | E | W | L | R | K | K | L | Q | D | V | H | N | F | | | -NH₂ |
| 22 | 16 | E | R | V | E | W | L | R | K | K | L | Q | D | V | H | N | F | | | -NH₂ |
| 23 | 17 | E | R | V | E | W | L | R | K | K | L | Q | D | V | H | N | F | | | -NH₂ |
| 24 | 18 | E | R | V | E | W | L | R | K | K | L | Q | D | V | H | N | F | | | -NH₂ |
| 25 | 19 | E | R | V | E | W | L | R | K | K | L | Q | D | V | H | N | F | | | -NH₂ |
| 46 | 40 | E | R | V | $ | W | L | R | K | K | L | Q | D | V | H | N | F | | | -NH₂ |
| 48 | 42 | E | R | $ | E | W | L | R | K | K | L | Q | D | V | H | N | F | | | -NH₂ |
| 64 | 58 | E | R | V | E | W | L | R | $ | K | L | Q | $ | V | H | N | F | | | -NH₂ |
| 67 | 61 | E | R | V | $ | W | L | R | $ | K | L | Q | D | V | H | N | F | | | -NH₂ |
| 68 | 62 | E | R | V | $ | W | L | R | $ | K | L | Q | D | V | H | N | F | | | -NH₂ |
| 72 | 66 | R | R | R | $ | W | L | H | $ | L | I | A | E | I | H | T | A | E | Y | -NH₂ |
| 73 | 67 | R | R | R | $ | W | L | H | $ | L | I | A | E | I | H | T | A | E | Y | -NH₂ |
| 78 | 72 | R | R | R | $ | W | L | R | $ | K | L | Q | D | V | H | N | Y | | | -NH₂ |
| 49 | 43 | E | R | $ | E | W | L | R | K | K | L | Q | D | V | H | N | F | | | -NH₂ |
| 77 | 71 | R | R | R | $ | W | L | R | $ | K | L | Q | D | V | H | N | Y | | | -NH₂ |
| 85 | 79 | R | R | R | $ | W | L | H | $ | L | I | A | E | I | H | T | A | | | -NH₂ |
| 89 | 83 | R | R | R | F | W | L | H | H | $ | I | A | E | $ | H | T | A | E | Y | -NH₂ |
| 86 | 80 | R | R | R | F | W | $ | H | H | A | $ | Q | E | I | H | T | A | E | Y | -NH₂ |
| 74 | 68 | R | R | R | $ | W | L | H | $ | A | I | Q | E | I | H | T | A | E | Y | -NH₂ |
| 123 | 117 | E | R | V | E | W | L | R | K | K | L | Q | D | V | H | N | F | | | -NH₂ |
| 124 | 118 | 74 | E | R | V | E | W | L | R | K | K | L | Q | D | V | H | N | F | | -NH₂ |
| 80 | 73 | E | R | V | E | W | L | R | K | K | L | Q | D | V | H | N | F | | | -NH₂ |
| 79 | 73 | E | R | V | E | W | L | R | K | K | L | Q | D | V | H | N | F | | | -NH₂ |
| 82 | 76 | E | R | V | E | W | L | R | K | K | L | Q | D | V | H | N | F | | | -NH₂ |

Table 2b
Linear Peptidomimetic

| SEQ | | | | | | | | | | | | | | | | | | | | |
|---|---|---|---|---|---|---|---|---|---|---|---|---|---|---|---|---|---|---|---|---|
| ID | SP# | 0 | 1 | 2 | 3 | 4 | 5 | 6 | 7 | 8 | 9 | 10 | 11 | 12 | 13 | 14 | 15 | 16 | 17 | 18 |
| 151 | 145 | H- | | | | | | | L | L | H | D | K | G | K | $ | I | Q | D | $ |
| 218 | 212 | H- | | | | | | | L | L | H | D | K | A | K | S | I | Q | D | $ |
| 219 | 213 | H- | | | | | | | L | L | H | D | K | A | K | S | I | Q | D | $ |
| 220 | 214 | H- | | | | | | | L | L | H | Q | R | A | K | W | L | N | S | $ |
| 224 | 218 | Ac- | | | | | | | L | L | H | D | K | A | K | S | I | Q | D | $ |
| 225 | 219 | Ac- | | | | | | | L | L | H | D | K | A | K | S | I | Q | D | $ |
| 226 | 220 | Ac- | | | | | | | L | L | H | Q | R | A | K | W | L | N | S | $ |
| 382 | 364 | Ac- | | | | | | | L | L | H | Q | R | A | K | W | L | N | S | $ |
| 383 | 365 | Ac- | | | | | | | L | L | H | Q | R | A | K | W | $r8 | N | S | L |
| 384 | 366 | Ac- | | | | | | | L | L | H | D | K | A | K | S | $r8 | Q | D | L |
| 385 | 379 | Ac- | | | | | | | L | L | H | D | K | A | K | S | $r8 | Q | D | L |
| 386 | 380 | Ac- | | | | | | | L | L | H | Q | R | A | K | W | $r8 | N | S | L |
| 387 | 381 | Ac- | | | | | | | L | L | H | Q | R | A | K | W | $r8 | N | S | L |
| 388 | 383 | Ac- | | | | | | | L | L | H | Q | R | A | $ | W | L | N | S | $ |
| 388 | 583 | Ac- | | | | | | | L | L | $ | Q | R | A | $ | W | I | N | S | $ |
| 390 | 385 | Ac- | | | | | | | L | L | $ | Q | R | A | $ | W | L | N | S | $ |
| 390 | 585 | Ac- | | | | | | | L | L | $ | Q | R | A | $ | W | L | N | S | $ |
| 392 | 387 | Ac- | | | | | | | L | L | $ | Q | R | A | $ | W | I | Q | D | $ |
| 391 | 386 | Ac- | | | | | | | L | L | $ | D | K | A | $ | S | I | Q | D | $ |
| 391 | 586 | Ac- | | | | | | | L | L | $ | D | K | A | $ | S | I | Q | D | $ |
| 393 | 388 | Ac- | | | | | | | L | L | $ | Q | R | A | $ | W | L | N | S | $ |
| 394 | 389 | Ac- | | | | | | | L | L | H | Q | R | A | K | W | $ | N | S | L |
| 395 | 390 | Ac- | | | | | | | L | L | H | D | K | A | K | S | $ | Q | D | L |
| 396 | 391 | Ac- | | | | | | | L | L | H | D | K | A | K | S | $ | Q | D | L |
| 397 | 392 | Ac- | | | | | | | L | L | H | Q | R | A | K | W | $ | N | S | L |
| 398 | 393 | Ac- | | | | | | | L | L | H | Q | R | A | K | W | $ | N | S | L |

| SEQ | | | | | | | | | | | | | | | | | | | | |
|---|---|---|---|---|---|---|---|---|---|---|---|---|---|---|---|---|---|---|---|---|
| ID | SP# | 19 | 20 | 21 | 22 | 23 | 24 | 25 | 26 | 27 | 28 | 29 | 30 | 31 | 32 | 33 | 34 | 35 | 36 | 37 |
| 151 | 145 | R | R | R | $ | W | L | H | $ | L | I | A | E | I | H | T | A | E | Y | NH₂ |
| 218 | 212 | R | R | R | $ | W | L | R | K | K | L | Q | D | V | H | N | F | | | NH₂ |
| 219 | 213 | R | R | R | $ | F | L | H | H | L | I | A | E | I | H | T | A | | | NH₂ |
| 220 | 214 | R | R | V | $ | W | L | R | K | K | L | Q | D | V | H | N | F | | | NH₂ |
| 224 | 218 | R | R | R | $ | W | L | R | K | K | L | Q | D | V | H | N | F | | | NH₂ |
| 225 | 219 | R | R | R | $ | F | L | H | H | L | I | A | E | I | H | T | A | | | NH₂ |
| 226 | 220 | R | R | V | $ | W | L | R | K | K | L | Q | D | V | H | N | F | | | NH₂ |
| 382 | 364 | E | R | V | $ | W | L | R | K | K | L | Q | D | V | H | E | F | | | NH₂ |
| 383 | 365 | E | R | V | $ | W | L | R | K | K | L | Q | D | V | H | E | F | | | NH₂ |
| 384 | 366 | R | R | R | $ | F | L | H | H | L | I | A | E | I | H | T | A | | | NH₂ |
| 385 | 379 | R | R | R | $ | W | L | R | K | K | L | Q | D | V | H | E | F | | | NH₂ |
| 386 | 380 | R | R | R | $ | W | L | R | K | K | L | Q | D | V | H | E | F | | | NH₂ |
| 387 | 381 | R | R | V | $ | W | L | R | K | K | L | Q | D | V | H | E | F | | | NH₂ |
| 388 | 383 | E | R | V | $ | W | L | R | Aib | K | L | Q | D | V | H | E | F | | | NH₂ |
| 388 | 583 | E | R | V | $ | W | L | R | K | K | L | Q | D | V | H | E | F | | | NH₂ |
| 390 | 385 | R | R | R | $ | W | L | R | Aib | K | L | Q | D | V | H | E | F | | | NH₂ |
| 390 | 585 | R | R | R | $ | W | L | R | K | K | L | Q | D | V | H | E | F | | | NH₂ |
| 391 | 386 | R | R | R | $ | F | L | H | H | L | I | A | E | I | H | T | A | | | NH₂ |
| 391 | 586 | R | R | R | $ | F | L | H | K | L | I | A | E | I | H | T | A | | | NH₂ |
| 392 | 387 | R | R | R | $ | W | L | R | K | K | L | Q | D | V | H | E | F | | | |
| 393 | 388 | R | R | R | $ | W | L | R | K | K | L | Q | D | V | H | E | F | | | |
| 394 | 389 | E | R | V | $r8 | W | L | R | K | K | L | Q | D | V | H | E | F | | | |
| 395 | 390 | R | R | R | $r8 | F | L | H | H | L | I | A | E | I | H | T | A | | | |
| 396 | 391 | R | R | R | $r8 | W | L | R | K | K | L | Q | D | V | H | E | F | | | |
| 397 | 392 | R | R | R | $r8 | W | L | R | K | K | L | Q | D | V | H | E | F | | | |
| 398 | 393 | R | R | V | $r8 | W | L | R | K | K | L | Q | D | V | H | E | F | | | |

TABLE 4
Exemplary Peptidomimetic Macrocycle Amino Acid Mutations

| SEQ | | | | | | | | | | | | | | | | | | | | |
|---|---|---|---|---|---|---|---|---|---|---|---|---|---|---|---|---|---|---|---|---|
| ID | SP# | 0 | 1 | 2 | 3 | 4 | 5 | 6 | 7 | 8 | 9 | 10 | 11 | 12 | 13 | 14 | 15 | 16 | 17 | 18 |
| 6 | PTH | H- | S | V | S | E | I | Q | L | M | H | N | L | G | K | H | L | N | S | M |
| 7 | PTHrP | H- | A | V | S | E | H | Q | L | L | H | D | K | G | K | S | I | Q | D | L |
| | | Ac- | Deg | W | Deg | Q | K | K | K | Nle | Ar | Q | hR | D- | S | W | hpb | K | E | Nle |
| | | | | | | | | | | | | | | Trp | | | | | | |
| | | 1NaAc- | Aib | R | Aib | W | E | E | E | L | Aib | A | R | A | +ch | Ar | Y | A | b- | hpb |
| | | | | | | | | | | | | | | | | | | | Ala | |
| | | 2NaAc- | Ac5c | w | Ac5c | F | F | F | A | hpb | | D | hpb | D- | K | A | $/ | E | B- | h- |
| | | | | | | | | | | | | | | AAs | | | | | hPhe | hIle |
| | | PhAc- | des- | r | E | H | I | A | F | $/ | | E | +ch | hpb | A | L | Aib | | Aib | hSER |
| | | | Ser | D-Arg | | | | | | | | | | | | | | | | OMe |
| | | FA | Ac3C | F4COOH | K | | | | F4Cl | | | K | Ar | Ar | L | K | | | | b- |
| | | | | | | | | | | | | | | | | | | | | hPhe |
| | | PEG | Ac6c | Bip | F | | | | 1Nal | | | A | L | R | E | R | | | | Aib |
| | | Ar | des- | F4NH₂ | Ac3c | | | | 2Nal | | | S | A | H | Q | F | | | | |
| | | | Ac5c | | | | | | | | | | | | | | | | | |
| | | | des- | 1Nal | Ac6c | | | | 2Pal | | | V | V | W | R | Aib | | | | |
| | | | Aib | | | | | | | | | | | | | | | | | |
| | | | V | 2Nal | G | | | | 3Pal | | | H | I | Aib | H | | | | | |
| | | | | 2Pal | A | | | | hpb | | | W | M | +ch | F | | | | | |
| | | | | 3Pal | | | | | Ar | | | $/ | K | $/ | W | | | | | |
| | | | | Ar | | | | | 4Pal | | | Aib | F | | P | | | | | |
| | | | | 4Pal | | | | | Phe | | | | W | | Aib | | | | | |
| | | | | Bpa | | | | | $/ | | | | w | | $/ | | | | | |
| | | | | Deg | | | | | Nle | | | | $/ | | Kfam | | | | | |
| | | | | I | | | | | M | | | | Nle | | Ktam | | | | | |
| | | | | | | | | | L | | | | Cit | | Cit | | | | | |
| | | | | | | | | | L | | | | hK | | | | | | | |
| | | | | | | | | | L | | | | hl | | | | | | | |

| SEQ | | | | | | | | | | | | | | | | | | | |
|---|---|---|---|---|---|---|---|---|---|---|---|---|---|---|---|---|---|---|---|
| ID | 19 | 20 | 21 | 22 | 23 | 24 | 25 | 26 | 27 | 28 | 29 | 30 | 31 | 32 | 33 | 34 | 35 | 36 | 37 |
| 6 | E | R | V | E | W | L | R | K | K | L | Q | D | V | H | N | F | | | -NH₂ |
| 7 | R | R | R | F | F | L | H | H | L | I | A | E | I | H | T | A | E | Y | OH |
| | S | Cit | R | Ar | 9- | Ar | L | +ch | R | Ar | E | L | Nle | W | E | Y | G | Ar | OH |
| | | | | | Aa1 | | | | | | | | | | | | | | |
| | +ch | +ch | K | E | 1Nal | hpb | W | K | Nle | hpb | S | R | T | R | D | R | | Pra | -NH₂ |
| | b- | A | Q | S | 2Nal | Ala | Y | S | Y | $/ | Aib | hF | S | F | K | 2NAl | | | -FA |
| | hLys | | | | | | | | | | | | | | | | | | |
| | Cit | | Cit | Aib | W | $/ | F | A | H | Cba | N | D | Ar | Y | F | hF | | | PEG |
| | Aib | | +ch | | Ar | Cba | Cit | E | F | Cpg | E | | hpb | I | | Ar | | | NHR |
| | A | | A | | hpb | Cpg | +ch | F | hF | Aib | H | | $/ | 2Pal | | hpb | | | NR′R″ |
| | | | | | A | Aib | A | $/ | M | Cha | S | | Cba | 3Pal | | E | | | OR |
| | | | | | | | S | Aib | Q | | A | | Cpg | Ar | | K | | | |
| | | | | | | | E | Cit | Cit | | F | | | 4Pal | | S | | | |
| | | | | | | | Aib | AmO | +ch | | | | | $/ | | | | | |
| | | | | | | | $/ | AmK | L | | | | | A | | | | | |
| | | | | | | | | | $/ | | | | | | | | | | |

des = desamino; Ar = Aromatic amino acids; hpb = Hydrophobic amino acids; +ch = Positively charged amino acids. OR = ester, aliphatic aromatic; NR′R″ = tertiary amide, aliphatic, aromatic; NHR = secondary amide, aliphatic, aromatic; FA = extesnion with fatty acid; PEG = extension with PEG,?

TABLE 5

| SEQ | | | | | | | | | | | | | | | | | | | | |
|---|---|---|---|---|---|---|---|---|---|---|---|---|---|---|---|---|---|---|---|---|
| ID | SP# | 0 | 1 | 2 | 3 | 4 | 5 | 6 | 7 | 8 | 9 | 10 | 11 | 12 | 13 | 14 | 15 | 16 | 17 | 18 |
| 6 | PTH | H- | S | V | S | E | I | Q | L | M | H | N | L | G | K | H | L | N | S | M |
| 7 | PTHrP | H- | A | V | S | E | H | Q | L | L | H | D | K | G | K | S | I | Q | D | L |
| 246 | 240 | Ac- | | | | | | | L | L | H | Q | R | A | K | $ | L | N | S | $ |
| 247 | 241 | Ac- | | | | | | | L | L | H | Q | R | A | $ | W | L | N | $ | L |
| 248 | 242 | Ac- | | | | | | | L | L | H | Q | R | A | $ | W | L | N | $ | L |
| 249 | 243 | Ac- | | | | | | | L | L | H | Q | R | A | K | W | L | N | S | $ |
| 250 | 244 | Ac- | | | | | | | L | L | H | Q | R | A | K | $ | I | Q | D | $ |
| 251 | 245 | Ac- | | | | | | | L | L | H | Q | R | A | $ | W | I | Q | $ | L |
| 252 | 246 | Ac- | | | | | | | L | L | H | Q | R | A | $ | W | I | Q | $ | L |
| 253 | 247 | Ac- | | | | | | | L | L | H | Q | R | A | K | W | I | Q | D | $ |
| 262 | 256 | Ac- | | | | | | | L | L | H | Q | R | A | K | W | I | Q | D | $ |
| 263 | 257 | Ac- | | | | | | | L | L | H | Q | R | A | K | W | I | Q | D | $ |
| 264 | 258 | Ac- | | | | | | | L | L | H | Q | R | A | K | W | I | Q | D | $ |
| 265 | 259 | Ac- | | | | | | | L | L | H | Q | R | A | K | W | I | Q | D | $ |
| 266 | 260 | Ac- | | | | | | | L | L | H | Q | R | A | K | W | I | Q | D | $ |
| 267 | 261 | Ac- | | | | | | | L | L | H | Q | R | A | K | W | I | Q | D | $ |
| 268 | 262 | Ac- | | | | | | | L | L | H | Q | R | A | Cit | W | I | Q | D | $ |
| 269 | 263 | Ac- | | | | | | | L | L | H | Q | Cit | A | K | W | I | Q | D | $ |
| 270 | 264 | Ac- | | | | | | | L | L | H | Q | R | A | K | W | I | Q | D | $ |
| 271 | 265 | Ac- | | | | | | | L | L | H | Q | R | A | K | W | I | Q | D | $ |
| 306 | 300 | Ac- | | | | | | | L | L | H | Q | R | A | K | W | L | N | S | $ |
| 307 | 301 | Ac- | | | | | | | L | L | H | E | R | A | K | F | L | N | S | $ |
| 323 | 317 | Ac- | | | | | | | L | L | $ | A | R | A | $ | W | I | A | D | L |
| 324 | 318 | Ac- | | | | | | | L | L | $ | A | R | A | $ | W | I | A | D | L |
| 325 | 319 | Ac- | | | | | | | L | L | H | Q | R | A | $ | W | I | Q | $ | L |
| 326 | 320 | Ac- | | | | | | | L | L | H | Q | R | A | $ | W | I | Q | $ | L |
| 327 | 321 | Ac- | | | | | | | L | L | H | A | R | A | $ | W | I | Q | $ | L |
| 328 | 322 | Ac- | | | | | | | L | L | H | A | R | A | $ | W | I | Q | $ | L |
| 329 | 323 | Ac- | | | | | | | L | L | H | Q | R | A | $ | W | I | A | $ | L |
| 330 | 324 | Ac- | | | | | | | L | L | H | Q | R | A | $ | W | I | A | $ | L |
| 331 | 325 | Ac- | | | | | | | L | L | H | A | R | A | $ | W | I | A | $ | L |
| 332 | 326 | Ac- | | | | | | | L | L | H | A | R | A | $ | W | I | A | $ | L |
| 333 | 327 | Ac- | | | | | | | L | L | $ | Q | R | A | $ | W | I | A | D | L |
| 334 | 328 | Ac- | | | | | | | L | L | $ | Q | R | A | $ | W | I | A | D | L |
| 335 | 329 | Ac- | | | | | | | L | L | $ | Q | R | A | $ | W | I | Q | D | $ |
| 336 | 330 | Ac- | | | | | | | L | L | $ | Q | R | A | $ | W | I | Q | D | L |
| 337 | 331 | Ac- | | | | | | | L | L | $ | Q | R | A | $ | W | I | Q | D | L |
| 338 | 332 | Ac- | | | | | | | L | L | $ | Q | R | A | $ | W | I | Q | D | L |
| 339 | 333 | Ac- | | | | | | | L | L | H | $ | R | A | K | $ | I | Q | D | L |
| 340 | 334 | Ac- | | | | | | | L | L | $ | Q | R | A | $ | W | I | Q | D | L |
| 341 | 335 | Ac- | | | | | | | L | L | $ | Q | R | A | $ | W | I | Q | D | L |
| 342 | 336 | Ac- | | | | | | | L | L | $ | A | R | A | $ | W | I | Q | D | $ |
| 343 | 337 | Ac- | | | | | | | L | L | $ | A | R | A | $ | W | I | Q | D | L |
| 344 | 338 | Ac- | | | | | | | L | L | $ | A | R | A | $ | W | I | Q | D | L |
| 345 | 339 | Ac- | | | | | | | L | L | $ | Q | R | A | $ | W | I | A | D | $ |
| 346 | 340 | Ac- | | | | | | | L | L | $ | A | R | A | $ | W | I | A | D | $ |
| 542 | 341 | Ac- | | | | | | | L | L | $ | Q | R | A | $ | W | I | Q | D | $ |
| 543 | 342 | Ac- | | | | | | | L | L | $ | Q | R | A | $ | W | I | Q | D | $ |
| 350 | 344 | Ac- | | | | | | | L | L | H | Q | R | A | $ | W | I | Q | $ | L |
| 351 | 345 | Ac- | | | | | | | L | L | $ | Q | R | A | $ | W | I | A | D | $ |
| 352 | 346 | Ac- | | | | | | | L | L | H | Q | R | A | $ | W | I | A | $ | L |
| 353 | 347 | Ac- | | | | | | | L | L | $ | A | R | A | $ | W | I | Q | D | $ |
| 354 | 348 | Ac- | | | | | | | L | L | H | A | R | A | $ | W | I | Q | $ | L |
| 355 | 349 | Ac- | | | | | | | L | L | $ | A | R | A | $ | W | I | A | D | $ |
| 356 | 350 | Ac- | | | | | | | L | L | H | A | R | A | $ | W | I | A | $ | L |
| 357 | 351 | Ac- | | | | | | | L | L | H | Q | R | A | K | W | I | Q | D | $ |
| 358 | 352 | Ac- | | | | | | | L | L | H | Q | R | A | K | W | I | Q | D | $ |
| 359 | 353 | Ac- | | | | | | | L | L | H | Q | R | A | K | W | I | Q | D | $ |
| 360 | 354 | Ac- | | | | | | | L | L | H | Q | R | A | K | W | I | Q | D | $ |
| 361 | 355 | Ac- | | | | | | | L | L | H | Q | R | A | K | W | I | Q | D | $ |
| 362 | 356 | Ac- | | | | | | | L | L | H | Q | R | A | K | W | I | Q | D | L |
| 363 | 357 | Ac- | | | | | | | L | L | H | Q | R | A | K | W | I | Q | D | $ |
| 364 | 358 | Ac- | | | | | | | L | L | H | Q | R | A | K | W | I | Q | D | $ |
| 366 | 360 | Ac- | | | | | | | L | L | H | Q | R | A | $ | W | I | Q | $ | L |
| 368 | 362 | Ac- | | | | | | | L | L | H | Q | R | A | $ | W | I | Q | $ | L |
| 389 | 384 | Ac- | | | | | | | L | L | $ | D | K | A | $ | S | I | Q | D | $ |
| 389 | 584 | Ac- | | | | | | | L | L | $ | D | K | A | $ | S | I | Q | D | $ |
| 392 | 387 | Ac- | | | | | | | L | L | $ | Q | R | A | $ | W | I | Q | D | $ |
| 416 | 412 | Ac- | | | | | | | L | L | H | Q | R | Aib | K | W | I | Q | D | $ |
| 417 | 413 | Ac- | | | | | | | L | L | H | Q | R | Aib | K | W | I | Q | D | $ |
| 418 | 414 | Ac- | | | | | | | L | L | H | Q | R | Aib | K | W | I | Q | D | $ |
| 419 | 415 | Ac- | | | | | | | L | L | H | Q | R | Aib | K | W | I | Q | D | $ |
| 420 | 416 | Ac- | | | | | | | L | L | H | Q | R | Aib | K | W | I | Q | D | $ |
| 421 | 417 | Ac- | | | | | | | L | L | H | Q | R | Aib | K | W | I | Q | D | $ |
| 422 | 418 | Ac- | | | | | | | L | L | H | Q | R | Aib | K | W | I | Q | D | $ |
| 423 | 419 | Ac- | | | | | | | L | L | H | Q | R | Aib | K | W | I | Q | D | $ |
| 424 | 420 | Ac- | | | | | | | L | L | H | Q | R | Aib | K | W | I | Q | D | $ |
| 425 | 421 | Ac- | | | | | | | L | L | H | Q | R | Aib | K | W | I | Q | D | $ |
| 426 | 422 | Ac- | | | | | | | L | L | H | Q | R | Aib | K | W | I | Q | D | $ |
| 427 | 423 | Ac- | | | | | | | L | L | H | Q | R | D- | K | W | I | Q | D | $ |
| | | | | | | | | | | | | | | Trp | | | | | | |
| 428 | 424 | Ac- | | | | | | | L | L | H | Q | R | Aib | K | W | $r8 | Q | D | L |
| 429 | 425 | Ac- | | | | | | | L | L | H | Q | R | Aib | K | W | $ | Q | D | L |
| 430 | 426 | Ac- | | | | | | | L | L | W | Q | R | Aib | K | W | $ | Q | D | L |
| 431 | 427 | Ac- | | | | | | | L | L | H | Q | R | Aib | K | W | $r8 | Q | D | L |
| 432 | 428 | Ac- | | | | | | | L | L | H | Q | R | A | K | W | I | Q | D | $ |
| 433 | 429 | Ac- | | | | | | | L | L | H | Q | R | A | K | D- | I | Q | D | $ |
| | | | | | | | | | | | | | | | | Trp | | | | |
| 434 | 430 | Ac- | | | | | | | L | L | H | Q | R | A | K | W | $r8 | Q | D | L |
| 435 | 431 | Ac- | | | | | | | L | L | $ | Q | R | A | $ | W | L | N | S | $ |
| 436 | 432 | Ac- | | | | | | | L | L | $ | Q | R | A | $ | W | L | N | S | $ |
| 437 | 433 | Ac- | | | | | | | L | L | $ | Q | R | A | $ | W | I | Q | D | $ |
| 438 | 434 | Ac- | | | | | | | L | L | H | Q | R | A | $ | W | I | Q | $ | L |
| 439 | 435 | Ac- | | | | | | | L | L | $ | Q | R | A | $ | W | I | Q | D | L |
| 440 | 436 | Ac- | | | | | | | L | L | $ | Q | R | A | $ | W | I | Q | D | L |
| 441 | 437 | Ac- | | | | | | | L | L | H | Q | R | A | $ | W | I | Q | $ | L |
| 442 | 438 | Ac- | | | | | | | L | L | H | Q | R | A | K | $ | I | Q | D | $ |
| 443 | 439 | Ac- | | | | | | | L | L | H | $ | R | A | K | $ | I | Q | D | L |
| 444 | 440 | Ac- | | | | | | | L | L | $ | Q | R | A | $ | W | I | Q | D | $ |
| 445 | 441 | Ac- | | | | | | | L | L | $ | Q | R | A | $ | W | I | Q | D | L |
| SEQ | | | | | | | | | | | | | | | | | | | | |
| ID | SP# | 19 | 20 | 21 | 22 | 23 | 24 | 25 | 26 | 27 | 28 | 29 | 30 | 31 | 32 | 33 | 34 | 35 | 36 | 37 |
| 6 | PTH | E | R | V | E | W | L | R | K | K | L | Q | D | V | H | N | F | | | -NH₂ |
| 7 | PTHRP | R | R | R | F | F | L | H | H | L | I | A | E | I | H | T | A | E | Y | -NH₂ |
| 246 | 240 | R | R | R | E | W | $ | R | K | K | $ | Q | D | V | H | N | F | | | -NH₂ |
| 247 | 241 | R | R | R | E | W | $ | R | K | K | $ | Q | D | V | H | N | F | | | -NH₂ |
| 248 | 242 | R | R | R | $ | W | L | R | $ | K | L | Q | D | V | H | N | F | | | -NH₂ |
| 249 | 243 | R | R | R | $ | W | L | R | K | K | L | Q | D | V | H | N | F | | | -NH₂ |
| 250 | 244 | R | R | R | E | W | $ | R | K | K | $ | Q | D | V | H | N | F | | | -NH₂ |
| 251 | 245 | R | R | R | E | W | $ | R | K | K | $ | Q | D | V | H | N | F | | | -NH₂ |
| 252 | 246 | R | R | R | $ | W | L | R | $ | K | L | Q | D | V | H | N | F | | | -NH₂ |
| 253 | 247 | R | R | R | $ | W | L | R | K | K | L | Q | D | V | H | N | F | | | -NH₂ |
| 262 | 256 | R | R | R | $ | W | L | R | K | Cit | L | Q | D | V | H | N | F | | | -NH₂ |
| 263 | 257 | R | R | R | $ | W | L | R | Cit | L | L | Q | D | V | H | N | F | | | -NH₂ |
| 264 | 258 | R | R | R | $ | W | L | Cit | K | L | L | Q | D | V | H | N | F | | | -NH₂ |
| 265 | 259 | R | R | Cit | $ | W | L | R | K | L | L | Q | D | V | H | N | F | | | -NH₂ |
| 266 | 260 | R | Cit | R | $ | W | L | R | K | L | L | Q | D | V | H | N | F | | | -NH₂ |
| 267 | 261 | Cit | R | R | V | W | L | R | K | L | L | Q | D | V | H | N | F | | | -NH₂ |
| 268 | 262 | R | R | R | $ | W | L | R | K | L | L | Q | D | V | H | N | F | | | -NH₂ |
| 269 | 263 | R | R | R | $ | W | L | R | K | L | L | Q | D | V | H | N | F | | | -NH₂ |
| 270 | 264 | R | R | R | $ | W | L | R | K | L | L | Q | F | V | H | N | F | | | -NH₂ |
| 271 | 265 | R | R | R | $ | W | L | R | K | L | L | Q | D | V | Y | N | F | | | -NH₂ |
| 306 | 300 | R | R | R | $ | W | L | R | K | K | L | Q | D | V | H | N | F | | | -NH₂ |
| 307 | 301 | R | R | R | $ | F | L | R | K | K | L | Q | D | V | H | N | F | | | -NH₂ |
| 323 | 317 | R | R | R | $ | W | L | R | $ | L | L | Q | D | V | H | N | F | | | -NH₂ |
| 324 | 318 | R | R | R | E | W | $ | R | K | L | $ | Q | D | V | H | N | F | | | -NH₂ |
| 325 | 319 | R | R | R | E | W | $ | R | K | L | $ | Q | D | V | H | N | F | | | -NH₂ |
| 326 | 320 | R | R | R | $ | W | L | R | $ | L | L | Q | D | V | H | N | F | | | -NH₂ |
| 327 | 321 | R | R | R | E | W | $ | R | K | L | $ | Q | D | V | H | N | F | | | -NH₂ |
| 328 | 322 | R | R | R | $ | W | L | R | $ | L | L | Q | D | V | H | N | F | | | -NH₂ |
| 329 | 323 | R | R | R | E | W | $ | R | K | L | $ | Q | D | V | H | N | F | | | -NH₂ |
| 330 | 324 | R | R | R | $ | W | L | R | $ | L | L | Q | D | V | H | N | F | | | -NH₂ |
| 331 | 325 | R | R | R | E | W | $ | R | K | L | $ | Q | D | V | H | N | F | | | -NH₂ |
| 332 | 326 | R | R | R | $ | W | L | R | $ | L | L | Q | D | V | H | N | F | | | -NH₂ |
| 333 | 327 | R | R | R | $ | W | L | R | $ | L | L | Q | D | V | H | N | F | | | -NH₂ |
| 334 | 328 | R | R | R | E | W | $ | R | K | L | $ | Q | D | V | H | N | F | | | -NH₂ |
| 335 | 329 | R | R | R | $ | W | L | R | Aib | K | L | Q | D | V | H | N | F | | | -NH₂ |
| 336 | 330 | R | R | R | $ | W | L | R | $ | K | L | Q | D | V | H | N | F | | | -NH₂ |
| 337 | 331 | R | R | R | E | W | $ | R | K | K | $ | Q | D | V | H | N | F | | | -NH₂ |
| 338 | 332 | R | R | R | E | W | L | R | $ | K | L | Q | $ | V | H | N | F | | | -NH₂ |
| 339 | 333 | R | R | R | E | W | $ | R | K | K | $ | Q | D | V | H | N | F | | | -NH₂ |
| 340 | 334 | R | R | R | $ | W | L | R | $ | L | L | Q | D | V | H | N | F | | | -NH₂ |
| 335 | 341 | R | R | R | E | W | $ | R | K | L | $ | Q | D | V | H | N | F | | | -NH₂ |
| 342 | 336 | R | R | R | $ | W | L | R | Aib | K | L | Q | D | V | H | N | F | | | -NH₂ |
| 343 | 337 | R | R | R | $ | W | L | R | $ | L | L | Q | D | V | H | N | F | | | -NH₂ |
| 344 | 338 | R | R | R | E | W | $ | R | K | L | $ | Q | D | V | H | N | F | | | -NH₂ |
| 345 | 339 | R | R | R | $ | W | L | R | Aib | K | L | Q | D | V | H | N | F | | | -NH₂ |
| 346 | 340 | R | R | R | $ | W | L | R | Aib | K | L | Q | D | V | H | N | F | | | -NH₂ |
| 542 | 341 | R | R | R | $ | W | L | R | Aib | K | L | Q | D | V | | | | | | -NH₂ |
| 543 | 342 | R | R | R | $ | W | L | R | Aib | K | L | Q | D | V | H | E | F | | | -NH₂ |
| 350 | 344 | R | R | R | E | W | $ | R | K | L | $ | Q | D | V | H | E | F | | | -NH₂ |
| 351 | 345 | R | R | R | $ | W | L | R | Aib | K | L | Q | D | V | H | E | F | | | -NH₂ |
| 352 | 346 | R | R | R | E | W | $ | R | K | L | $ | Q | D | V | H | E | F | | | -NH₂ |
| 353 | 347 | R | R | R | $ | W | L | R | Aib | K | L | Q | D | V | H | E | F | | | -NH₂ |
| 354 | 348 | R | R | R | E | W | $ | R | K | L | $ | Q | D | V | H | E | F | | | -NH₂ |
| 355 | 349 | R | R | R | $ | W | L | R | Aib | K | L | Q | D | V | H | E | F | | | -NH₂ |
| 356 | 350 | R | R | R | E | W | $ | R | K | L | $ | Q | D | V | H | E | F | | | -NH₂ |
| 357 | 351 | R | R | R | $ | W | L | R | K | K | L | Q | D | V | H | N | F | | | -NH₂ |
| 358 | 352 | R | R | R | $ | W | L | R | K | L | L | Q | D | V | H | N | F | | | -NH₂ |
| 359 | 353 | R | R | R | $ | W | L | R | Aib | K | L | Q | D | V | H | N | F | | | -NH₂ |
| 360 | 354 | R | R | R | $ | W | L | R | Aib | L | L | Q | D | V | H | N | F | | | -NH₂ |
| 361 | 355 | R | R | R | $ | W | L | R | Cit | L | L | Q | D | V | H | N | F | | | -NH₂ |
| 362 | 356 | R | R | R | E | W | L | R | K | K | L | Q | D | V | H | N | F | | | -NH₂ |
| 363 | 357 | R | R | R | $ | W | L | R | Aib | K | L | Q | D | V | H | N | F | G | Pra | -NH₂ |
| 364 | 358 | R | R | R | $ | W | L | R | AmO | L | L | Q | D | V | H | N | F | | | -NH₂ |
| 366 | 360 | R | R | R | $ | W | L | R | $ | K | L | Q | D | V | H | N | F | | | -NH₂ |
| 368 | 362 | R | R | R | E | W | $ | R | K | K | $ | Q | D | V | H | N | F | | | -NH₂ |
| 389 | 384 | R | R | R | $ | W | L | R | Aib | K | L | Q | D | V | H | E | F | | | -NH₂ |
| 389 | 584 | R | R | R | $ | W | L | R | K | K | L | Q | D | V | H | E | F | | | -NH₂ |
| 392 | 387 | R | R | R | $ | W | L | R | K | K | L | Q | D | V | H | E | F | | | -NH₂ |
| 416 | 412 | R | R | R | $ | W | L | R | Aib | L | L | Q | D | V | H | E | F | | | -NH₂ |
| 417 | 413 | R | R | R | $ | W | L | H | H | L | L | Q | D | V | H | E | F | | | -NH₂ |
| 418 | 414 | R | R | R | $ | W | L | H | K | L | L | Q | D | V | H | E | F | | | -NH₂ |
| 419 | 415 | R | R | R | $ | W | L | R | H | L | L | Q | D | V | H | E | F | | | -NH₂ |
| 420 | 416 | R | R | R | $ | W | L | Aib | K | L | L | Q | D | V | H | E | F | | | -NH₂ |
| 421 | 417 | R | R | R | $ | W | L | H | Aib | L | L | Q | D | V | H | E | F | | | -NH₂ |
| 422 | 418 | R | R | R | $ | W | L | A | H | L | L | Q | D | V | H | E | F | | | -NH₂ |
| 423 | 419 | R | R | R | $ | W | L | Aib | H | L | L | Q | D | V | H | E | F | | | -NH₂ |
| 424 | 420 | R | R | R | $ | W | L | R | K | L | L | Q | D | V | H | E | F | | | -NH₂ |
| 425 | 421 | R | R | R | $ | W | L | R | K | L | L | Q | D | V | H | E | F | | | -NH₂ |
| 426 | 422 | R | R | R | $ | W | L | R | K | L | L | Q | D | V | H | E | F | | | -NH₂ |
| 427 | 423 | R | R | R | $ | W | L | R | Aib | L | L | Q | D | V | H | E | F | | | -NH₂ |
| 428 | 424 | R | R | R | $ | W | L | R | Aib | L | L | Q | D | V | H | E | F | | | -NH₂ |
| 429 | 425 | R | R | R | $r8 | W | L | R | Aib | L | L | Q | D | V | H | E | F | | | -NH₂ |
| 430 | 426 | R | R | R | $r8 | W | L | R | Aib | K | L | Q | D | V | H | E | F | | | -NH₂ |
| 431 | 427 | R | R | R | $ | W | L | R | Aib | K | L | Q | D | V | H | E | F | | | -NH₂ |
| 432 | 428 | R | R | R | $ | W | L | R | K | K | L | Q | D | V | H | N | Y | | | -NH₂ |
| 433 | 429 | R | R | R | $ | W | L | R | K | K | L | Q | D | V | H | N | Y | | | -NH₂ |
| 434 | 430 | R | R | R | $ | W | L | R | K | K | L | Q | D | V | H | E | F | | | -NH₂ |
| 436 | 431 | R | R | V | $ | W | L | R | Aib | K | L | Q | D | V | H | E | F | | | -NH₂ |
| 436 | 432 | R | R | V | $ | W | L | R | K | K | L | Q | D | V | H | E | F | | | -NH₂ |
| 437 | 433 | R | R | R | $ | W | L | R | K | K | L | Q | D | V | H | E | F | | | -NH₂ |
| 438 | 434 | R | R | R | $ | W | L | R | $ | K | L | Q | D | V | H | E | F | | | -NH₂ |
| 439 | 435 | R | R | R | $ | W | L | R | $ | K | L | Q | D | V | H | E | F | | | -NH₂ |
| 440 | 436 | R | R | R | E | W | $ | R | K | L | $ | Q | D | V | H | E | F | | | -NH₂ |
| 441 | 437 | R | R | R | E | W | $ | R | K | K | $ | Q | D | V | H | E | F | | | -NH₂ |
| 442 | 438 | R | R | R | E | W | $ | R | K | K | $ | Q | D | V | H | E | F | | | -NH₂ |
| 443 | 439 | R | R | R | E | W | $ | R | K | K | $ | Q | D | V | H | E | F | | | -NH₂ |
| 444 | 440 | R | R | R | $ | W | L | R | K | K | L | Q | D | V | | | | | | -NH₂ |
| 445 | 441 | R | R | R | E | W | L | R | $ | K | L | Q | $ | V | H | E | F | | | -NH₂ |

TABLE 6

| SEQ | | | | | | | | | | | | | | | | | | | | |
|---|---|---|---|---|---|---|---|---|---|---|---|---|---|---|---|---|---|---|---|---|
| ID | SP# | 0 | 1 | 2 | 3 | 4 | 5 | 6 | 7 | 8 | 9 | 10 | 11 | 12 | 13 | 14 | 15 | 16 | 17 | 18 |
| 6 | PTH | H- | S | V | S | E | I | Q | L | M | H | N | L | G | K | H | L | N | S | M |
| 7 | PTHrP | H- | A | V | S | E | H | Q | L | L | H | D | K | G | K | S | I | Q | D | L |
| 357 | 351 | Ac- | | | | | | | L | L | H | Q | R | A | K | W | I | Q | D | $ |
| 359 | 353 | Ac- | | | | | | | L | L | H | Q | R | A | K | W | I | Q | D | $ |
| 360 | 354 | Ac- | | | | | | | L | L | H | Q | R | A | K | W | I | Q | D | $ |
| 544 | 355 | H | | | | | | | L | L | H | Q | R | A | K | W | I | Q | D | $ |
| 253 | 247 | H | | | | | | | L | L | H | Q | R | A | K | W | I | Q | D | $ |
| 265 | 259 | H | | | | | | | L | L | H | Q | R | A | K | W | I | Q | D | $ |
| 306 | 300 | H | | | | | | | L | L | H | Q | R | A | K | W | L | N | S | $ |
| 263 | 257 | H | | | | | | | L | L | H | Q | R | A | K | W | I | Q | D | $ |
| 268 | 262 | H | | | | | | | L | L | H | Q | R | A | Cit | W | I | Q | D | $ |
| 271 | 265 | H | | | | | | | L | L | H | Q | R | A | K | W | I | Q | D | $ |
| 307 | 301 | H | | | | | | | L | L | H | E | R | A | K | F | L | N | S | $ |
| 269 | 263 | H | | | | | | | L | L | H | Q | Cit | A | K | W | I | Q | D | $ |
| 264 | 258 | H | | | | | | | L | L | H | Q | R | A | K | W | I | Q | D | $ |
| 267 | 261 | H | | | | | | | L | L | H | Q | R | A | K | W | I | Q | D | $ |
| 270 | 264 | H | | | | | | | L | L | H | Q | R | A | K | W | I | Q | D | $ |
| 266 | 260 | H | | | | | | | L | L | H | Q | R | A | K | W | I | Q | D | $ |
| 545 | 256 | Ac- | | | | | | | L | L | H | Q | R | A | K | W | I | Q | D | $ |
| 358 | 352 | Ac- | | | | | | | L | L | H | Q | R | A | K | W | I | Q | D | $ |
| 546 | 357 | Ac- | | | | | | | L | L | H | Q | R | A | K | W | I | Q | D | $ |
| 364 | 358 | Ac- | | | | | | | L | L | H | Q | R | A | K | W | I | Q | D | $ |
| 416 | 412 | Ac- | | | | | | | L | L | H | Q | R | Aib | K | W | I | Q | D | $ |
| 417 | 413 | Ac- | | | | | | | L | L | H | Q | R | Aib | K | W | I | Q | D | $ |
| 418 | 414 | Ac- | | | | | | | L | L | H | Q | R | Aib | K | W | I | Q | D | $ |
| 419 | 415 | Ac- | | | | | | | L | L | H | Q | R | Aib | K | W | I | Q | D | $ |
| 420 | 416 | Ac- | | | | | | | L | L | H | Q | R | Aib | K | W | I | Q | D | $ |
| 421 | 417 | Ac- | | | | | | | L | L | H | Q | R | Aib | K | W | I | Q | D | $ |
| 422 | 418 | Ac- | | | | | | | L | L | H | Q | R | Aib | K | W | I | Q | D | $ |
| 423 | 419 | Ac- | | | | | | | L | L | H | Q | R | Aib | K | W | I | Q | D | $ |
| 424 | 420 | Ac- | | | | | | | L | L | H | Q | R | Aib | K | W | I | Q | D | $ |
| 425 | 421 | Ac- | | | | | | | L | L | H | Q | R | Aib | K | W | I | Q | D | $ |
| 426 | 422 | Ac- | | | | | | | L | L | H | Q | R | Aib | K | W | I | Q | D | $ |
| 427 | 423 | Ac- | | | | | | | L | L | H | Q | R | D- | K | W | I | Q | D | $ |
| | | | | | | | | | | | | | | Trp | | | | | | |
| 428 | 424 | Ac- | | | | | | | L | L | H | Q | R | Aib | K | W | $r8 | Q | D | L |
| 429 | 425 | Ac- | | | | | | | L | L | H | Q | R | Aib | K | W | $ | Q | D | L |
| 430 | 426 | Ac- | | | | | | | L | L | H | Q | R | Aib | K | W | $ | Q | D | L |
| 431 | 427 | Ac- | | | | | | | L | L | H | Q | R | Aib | K | W | $r8 | Q | D | L |
| 432 | 428 | Ac- | | | | | | | L | L | H | Q | R | A | K | W | I | Q | D | $ |
| 433 | 429 | Ac- | | | | | | | L | L | H | Q | R | A | K | D- | I | Q | D | $ |
| | | | | | | | | | | | | | | | | Trp | | | | |
| 434 | 430 | Ac- | | | | | | | L | L | H | Q | R | A | K | W | $r8 | Q | D | L |
| 547 | 432 | Ac- | | | | | | | L | L | $ | Q | R | A | $ | W | L | N | S | $ |
| 547 | 532 | Ac- | | | | | | | L | L | $ | Q | R | A | $ | W | L | N | S | $ |
| 349 | 343 | Ac- | | | | | | | L | L | $ | Q | R | A | $ | W | I | Q | D | $ |
| 351 | 345 | Ac- | | | | | | | L | L | $ | Q | R | A | $ | W | I | A | D | $ |
| 353 | 347 | Ac- | | | | | | | L | L | $ | A | R | A | $ | W | I | Q | D | $ |
| 355 | 349 | Ac- | | | | | | | L | L | $ | A | R | A | $ | W | I | A | D | $ |
| 366 | 360 | Ac- | | | | | | | L | L | H | Q | R | A | $ | W | I | Q | $ | L |
| 335 | 329 | Ac- | | | | | | | L | L | $ | Q | R | A | $ | W | I | Q | D | $ |
| 342 | 336 | Ac- | | | | | | | L | L | $ | A | R | A | $ | W | I | Q | D | $ |
| 346 | 340 | Ac- | | | | | | | L | L | $ | A | R | A | $ | W | I | A | D | $ |
| 345 | 339 | Ac- | | | | | | | L | L | $ | Q | R | A | $ | W | I | A | D | $ |
| 336 | 330 | Ac- | | | | | | | L | L | $ | Q | R | A | $ | W | I | Q | D | L |
| 338 | 332 | Ac- | | | | | | | L | L | $ | Q | R | A | $ | W | I | Q | D | L |
| 252 | 246 | H- | | | | | | | L | L | H | Q | R | A | $ | W | I | Q | $ | L |
| 341 | 335 | Ac- | | | | | | | L | L | $ | Q | R | A | $ | W | I | Q | D | L |
| 340 | 334 | Ac- | | | | | | | L | L | $ | Q | R | A | $ | W | I | Q | D | L |
| 368 | 362 | Ac- | | | | | | | L | L | H | Q | R | A | $ | W | I | Q | $ | L |
| 250 | 244 | H- | | | | | | | L | L | H | Q | R | A | K | $ | I | Q | D | $ |
| 337 | 331 | Ac- | | | | | | | L | L | $ | Q | R | A | $ | W | I | Q | D | L |
| 339 | 333 | Ac- | | | | | | | L | L | H | $ | R | A | K | $ | I | Q | D | L |
| 356 | 350 | Ac- | | | | | | | L | L | H | A | R | A | $ | W | I | A | $ | L |
| 344 | 338 | Ac- | | | | | | | L | L | $ | A | R | A | $ | W | I | Q | D | L |
| 251 | 245 | H- | | | | | | | L | L | H | Q | R | A | $ | W | I | Q | $ | L |
| 333 | 327 | Ac- | | | | | | | L | L | $ | Q | R | A | $ | W | I | A | D | L |
| 334 | 328 | Ac- | | | | | | | L | L | $ | Q | R | A | $ | W | I | A | D | L |
| 324 | 318 | Ac- | | | | | | | L | L | $ | A | R | A | $ | W | I | A | D | L |
| 327 | 321 | Ac- | | | | | | | L | L | H | A | R | A | $ | W | I | Q | $ | L |
| 354 | 348 | Ac- | | | | | | | L | L | H | A | R | A | $ | W | I | Q | $ | L |
| 325 | 319 | Ac- | | | | | | | L | L | H | Q | R | A | $ | W | I | Q | $ | L |
| 350 | 344 | Ac- | | | | | | | L | L | H | Q | R | A | $ | W | I | Q | $ | L |
| 328 | 322 | Ac- | | | | | | | L | L | H | A | R | A | $ | W | I | Q | $ | L |
| 331 | 325 | Ac- | | | | | | | L | L | H | S | T | S | $ | W | I | A | $ | L |
| 330 | 324 | Ac- | | | | | | | L | L | H | Q | R | A | $ | W | I | A | $ | L |
| 329 | 323 | Ac- | | | | | | | L | L | H | Q | R | A | $ | W | I | A | $ | L |
| 352 | 346 | Ac- | | | | | | | L | L | H | Q | R | A | $ | W | I | A | $ | L |
| 332 | 326 | Ac- | | | | | | | L | L | H | A | R | A | $ | W | I | A | $ | L |
| 323 | 317 | Ac- | | | | | | | L | L | $ | A | R | A | $ | W | I | A | D | L |
| 326 | 320 | Ac- | | | | | | | L | L | H | Q | R | A | $ | W | I | Q | $ | L |
| 343 | 337 | Ac- | | | | | | | L | L | $ | A | R | A | $ | W | I | Q | D | L |
| 389 | 384 | Ac- | | | | | | | L | L | $ | D | K | A | $ | S | I | Q | D | $ |
| 389 | 584 | Ac- | | | | | | | L | L | $ | D | K | A | $ | S | I | Q | D | $ |
| 437 | 433 | Ac- | | | | | | | L | L | $ | Q | R | A | $ | W | I | Q | D | $ |
| 438 | 434 | Ac- | | | | | | | L | L | H | Q | R | A | $ | W | I | Q | $ | L |
| 439 | 435 | Ac- | | | | | | | L | L | $ | Q | R | A | $ | W | I | Q | D | L |
| 440 | 436 | Ac- | | | | | | | L | L | $ | Q | R | A | $ | W | I | Q | D | L |
| 441 | 437 | Ac- | | | | | | | L | L | H | Q | R | A | $ | W | I | Q | $ | L |
| 442 | 438 | Ac- | | | | | | | L | L | H | Q | R | A | K | $ | I | Q | D | $ |
| 443 | 439 | Ac- | | | | | | | L | L | H | $ | R | A | K | $ | I | Q | D | L |
| 444 | 440 | Ac- | | | | | | | L | L | $ | Q | R | A | $ | W | I | Q | D | $ |
| 445 | 441 | Ac- | | | | | | | L | L | $ | Q | R | A | $ | W | I | Q | D | L |
| SEQ | | | | | | | | | | | | | | | | | | | | |
| ID | SP# | 19 | 20 | 21 | 22 | 23 | 24 | 25 | 26 | 27 | 28 | 29 | 30 | 31 | 32 | 33 | 34 | 35 | 36 | 37 |
| 6 | PTH | E | R | V | E | W | L | R | K | K | L | Q | D | V | H | N | F | | | -NH₂ |
| 7 | PTHRP | R | R | R | F | F | L | H | H | L | I | A | E | I | H | T | A | E | Y | -NH₂ |
| 357 | 351 | E | R | V | E | W | L | R | K | K | L | Q | D | V | H | N | F | | | -NH₂ |
| 359 | 353 | R | R | R | F | F | L | H | Aib | K | L | Q | D | V | H | N | F | | | -NH₂ |
| 360 | 354 | R | R | R | $ | W | L | R | Aib | L | L | Q | D | V | H | N | F | | | -NH₂ |
| 544 | 355 | R | R | R | $ | W | L | R | Cit | L | L | Q | D | V | H | N | F | | | -NH₂ |
| 253 | 247 | R | R | R | $ | W | L | R | K | K | L | Q | D | V | H | N | F | | | -NH₂ |
| 265 | 259 | R | R | R | $ | W | L | R | K | L | L | Q | D | V | H | N | F | | | -NH₂ |
| 306 | 300 | R | R | R | $ | W | L | R | K | K | L | Q | D | V | H | N | F | | | -NH₂ |
| 263 | 257 | R | R | Cit | $ | W | L | R | Cit | L | L | Q | D | V | H | N | F | | | -NH₂ |
| 268 | 262 | R | R | R | $ | W | L | R | K | L | L | Q | D | V | H | N | F | | | -NH₂ |
| 271 | 265 | R | R | R | $ | W | L | R | K | L | L | Q | D | V | Y | N | F | | | -NH₂ |
| 307 | 301 | R | R | R | $ | W | L | R | K | K | L | Q | D | V | H | N | F | | | -NH₂ |
| 269 | 263 | R | R | R | $ | W | L | R | K | L | L | Q | D | V | H | N | F | | | -NH₂ |
| 264 | 258 | R | R | R | $ | F | L | R | K | L | L | Q | D | V | H | N | F | | | -NH₂ |
| 267 | 261 | Cit | R | R | $ | W | L | R | K | L | L | Q | D | V | H | N | F | | | -NH₂ |
| 270 | 264 | R | R | R | $ | W | L | R | K | L | L | Q | F | V | H | N | F | | | -NH₂ |
| 266 | 260 | R | Cit | R | $ | W | L | R | K | L | L | Q | D | V | H | N | F | | | -NH₂ |
| 545 | 256 | R | R | R | $ | W | L | R | K | Cit | L | Q | D | V | H | N | F | | | -NH₂ |
| 358 | 352 | R | R | R | $ | W | L | R | K | L | L | Q | D | V | Y | N | F | | | -NH₂ |
| 546 | 357 | R | R | R | $ | W | L | R | Aib | K | L | Q | D | V | H | N | F | | | -NH₂ |
| 364 | 358 | R | R | R | $ | F | L | R | AmO | L | L | Q | D | V | H | N | F | | | -NH₂ |
| 416 | 412 | R | R | R | $ | W | L | R | Aib | L | L | Q | D | V | H | E | F | | | -NH₂ |
| 417 | 413 | R | R | R | $ | W | L | H | H | L | L | Q | D | V | H | E | F | | | -NH₂ |
| 418 | 414 | R | R | R | $ | W | L | H | K | L | L | Q | D | V | H | E | F | | | -NH₂ |
| 419 | 415 | R | R | R | $ | W | L | R | H | L | L | Q | D | V | H | E | F | | | -NH₂ |
| 420 | 416 | R | R | R | $ | W | L | Aib | K | L | L | Q | D | V | H | E | F | | | -NH₂ |
| 421 | 417 | R | R | R | $ | W | L | H | Aib | L | L | Q | D | V | H | E | F | | | -NH₂ |
| 422 | 418 | R | R | R | $ | W | L | A | H | L | L | Q | D | V | H | E | F | | | -NH₂ |
| 423 | 419 | R | R | R | $ | W | L | Aib | H | L | L | Q | D | V | H | E | F | | | -NH₂ |
| 424 | 420 | R | R | R | $ | W | L | r | K | L | L | Q | D | V | H | E | F | | | -NH₂ |
| 425 | 421 | R | R | R | $ | W | L | R | k | L | L | Q | D | V | H | E | F | | | -NH₂ |
| 426 | 422 | R | R | R | $ | W | L | r | k | L | L | Q | D | V | H | E | F | | | -NH₂ |
| 427 | 423 | R | R | R | $ | W | L | R | Aib | L | L | Q | D | V | H | E | F | | | -NH₂ |
| 428 | 424 | R | R | R | $ | W | L | R | Aib | L | L | Q | D | V | H | E | F | | | -NH₂ |
| 429 | 425 | R | R | R | $r8 | W | L | R | Aib | L | L | Q | D | V | H | E | F | | | -NH₂ |
| 430 | 426 | R | R | R | $r8 | W | L | R | Aib | K | L | Q | D | V | H | E | F | | | -NH₂ |
| 431 | 427 | R | R | R | $ | W | L | R | Aib | K | L | Q | D | V | H | E | F | | | -NH₂ |
| 432 | 428 | R | R | R | $ | W | L | R | K | K | L | Q | D | V | H | N | Y | | | -NH₂ |
| 433 | 429 | R | R | R | $ | W | L | R | K | K | L | Q | D | V | H | N | Y | | | -NH₂ |
| 434 | 430 | R | R | R | $ | W | L | R | K | K | L | Q | D | V | H | E | F | | | -NH₂ |
| 547 | 432 | R | R | V | $ | W | L | R | Aib | K | L | Q | D | V | H | E | F | | | -NH₂ |
| 547 | 532 | R | R | V | $ | W | L | R | K | K | L | Q | D | V | H | E | F | | | -NH₂ |
| 349 | 343 | R | R | R | $ | W | L | R | Aib | K | L | Q | D | V | H | E | F | | | -NH₂ |
| 351 | 345 | R | R | R | $ | W | L | R | Aib | K | L | Q | D | V | H | E | F | | | -NH₂ |
| 353 | 347 | R | R | R | $ | W | L | R | Aib | K | L | Q | D | V | H | E | F | | | -NH₂ |
| 355 | 349 | R | R | R | $ | W | L | R | Aib | K | L | Q | D | V | H | E | F | | | -NH₂ |
| 366 | 360 | R | R | R | $ | W | L | R | $ | K | L | Q | D | V | H | N | F | | | -NH₂ |
| 335 | 329 | R | R | R | $ | W | L | R | Aib | K | L | Q | D | V | H | N | F | | | -NH₂ |
| 342 | 336 | R | R | R | $ | W | L | R | Aib | K | L | Q | D | V | H | N | F | | | -NH₂ |
| 346 | 340 | R | R | R | $ | W | L | R | Aib | K | L | Q | D | V | H | N | F | | | -NH₂ |
| 345 | 339 | R | R | R | $ | W | L | R | Aib | K | L | Q | D | V | H | N | F | | | -NH₂ |
| 336 | 330 | R | R | R | $ | W | L | R | $ | K | $ | Q | D | V | H | N | F | | | -NH₂ |
| 338 | 332 | R | R | R | E | W | L | R | $ | K | L | Q | D | V | H | N | F | | | -NH₂ |
| 252 | 246 | R | R | R | $ | W | L | R | $ | K | $ | Q | D | V | H | N | F | | | -NH₂ |
| 341 | 335 | R | R | R | E | W | $ | R | K | L | L | Q | D | V | H | N | F | | | -NH₂ |
| 340 | 334 | R | R | R | $ | W | L | R | $ | L | L | Q | D | V | H | N | F | | | -NH₂ |
| 368 | 362 | R | R | R | E | W | $ | R | K | K | L | Q | D | V | H | N | F | | | -NH₂ |
| 250 | 244 | R | R | R | E | W | $ | R | K | K | L | Q | D | V | H | N | F | | | -NH₂ |
| 337 | 331 | R | R | R | E | W | $ | R | K | K | L | Q | D | V | H | N | F | | | -NH₂ |
| 339 | 333 | R | R | R | E | W | $ | R | K | K | L | Q | D | V | H | N | F | | | -NH₂ |
| 356 | 350 | R | R | R | E | W | $ | R | K | L | L | Q | D | V | H | E | F | | | -NH₂ |
| 344 | 338 | R | R | R | E | W | $ | R | K | L | L | Q | D | V | H | N | F | | | -NH₂ |
| 251 | 245 | R | R | R | E | W | $ | R | K | K | L | Q | D | V | H | N | F | | | -NH₂ |
| 333 | 327 | R | R | R | $ | W | L | R | $ | L | $ | Q | D | V | H | N | F | | | -NH₂ |
| 334 | 328 | R | R | R | E | W | $ | R | K | L | L | Q | D | V | H | N | F | | | -NH₂ |
| 324 | 318 | R | R | R | E | W | $ | R | K | L | L | Q | D | V | H | N | F | | | -NH₂ |
| 327 | 321 | R | R | R | E | W | $ | R | K | L | L | Q | D | V | H | N | F | | | -NH₂ |
| 354 | 348 | R | R | R | E | W | $ | R | K | L | L | Q | D | V | H | E | F | | | -NH₂ |
| 325 | 319 | R | R | R | E | W | $ | H | K | L | L | Q | D | V | H | N | F | | | -NH₂ |
| 350 | 344 | R | R | R | E | W | $ | H | K | L | L | Q | D | V | H | E | F | | | -NH₂ |
| 328 | 322 | R | R | R | $ | W | L | R | $ | L | L | Q | D | V | H | N | F | | | -NH₂ |
| 331 | 325 | R | R | R | E | W | $ | R | K | L | L | Q | D | V | H | N | F | | | -NH₂ |
| 330 | 324 | R | R | R | $ | W | L | R | $ | L | L | Q | D | V | H | N | F | | | -NH₂ |
| 329 | 323 | R | R | R | E | W | $ | A | K | L | L | Q | D | V | H | N | F | | | -NH₂ |
| 352 | 346 | R | R | R | E | W | $ | R | K | L | L | Q | D | V | H | E | F | | | -NH₂ |
| 332 | 326 | R | R | R | $ | W | L | R | $ | L | L | Q | D | V | H | N | F | | | -NH₂ |
| 323 | 317 | R | R | R | $ | W | L | R | $ | L | L | Q | D | V | H | N | F | | | -NH₂ |
| 326 | 320 | R | R | R | $ | W | L | R | $ | L | L | Q | D | V | H | N | F | | | -NH₂ |
| 343 | 337 | R | R | R | $ | W | L | R | $ | L | L | Q | D | V | H | N | F | | | -NH₂ |
| 389 | 384 | R | R | R | $ | W | L | R | K | K | L | Q | D | V | H | E | F | | | -NH₂ |
| 389 | 584 | R | R | R | $ | W | L | R | K | K | L | Q | D | V | H | E | F | | | -NH₂ |
| 437 | 433 | R | R | R | $ | W | L | R | $ | K | L | Q | D | V | H | E | F | | | -NH₂ |
| 438 | 434 | R | R | R | $ | W | L | R | $ | K | L | Q | D | V | H | E | F | | | -NH₂ |
| 439 | 435 | R | R | R | $ | W | L | R | K | K | L | Q | D | V | H | E | F | | | -NH₂ |
| 440 | 436 | R | R | R | E | W | $ | R | K | L | $ | Q | D | V | H | E | F | | | -NH₂ |
| 441 | 437 | R | R | R | E | W | $ | R | K | K | $ | Q | D | V | H | E | F | | | -NH₂ |
| 442 | 438 | R | R | R | E | W | $ | R | K | K | $ | Q | D | V | H | E | F | | | -NH₂ |
| 443 | 439 | R | R | R | E | W | $ | R | K | K | $ | Q | D | V | H | E | F | | | -NH₂ |
| 444 | 440 | R | R | R | $ | W | L | R | K | K | L | Q | D | V | | | | | | -NH₂ |
| 445 | 441 | R | R | R | E | W | L | R | $ | K | L | Q | D | V | H | E | F | | | -NH₂ |

TABLE 7

| SEQ | | | | | | | | | | | | | | | | | | | | |
|---|---|---|---|---|---|---|---|---|---|---|---|---|---|---|---|---|---|---|---|---|
| ID | SP# | 0 | 1 | 2 | 3 | 4 | 5 | 6 | 7 | 8 | 9 | 10 | 11 | 12 | 13 | 14 | 15 | 16 | 17 | 18 |
| 6 | PTH | H- | S | V | S | E | I | Q | L | M | H | N | L | G | K | H | L | N | S | M |
| 7 | PTHrP | H- | A | V | S | E | H | Q | L | L | H | D | K | G | K | S | I | Q | D | L |
| 83 | 77 | H- | | | | | | | L | L | H | Q | hR | A | K | $ | I | Q | D | $ |
| 98 | 92 | Hep- | | | | | | | L | L | H | Q | hR | A | K | W | I | Q | D | L |
| 84 | 78 | H- | | | | | | | L | L | H | Q | hR | A | K | W | I | Q | D | L |
| 91 | 85 | H- | | | | | | | L | L | H | Q | hR | A | K | $ | I | Q | D | $ |
| 90 | 84 | H- | | | | | | | L | L | H | Q | hR | A | K | $ | I | Q | D | $ |
| 124 | 118 | H- | | | | | | | L | L | H | Q | hR | A | K | $r5 | I | Q | D | $ |
| 163 | 157 | Hep- | | | | | | | L | L | H | Q | hR | A | K | W | L | Q | D | L |
| 164 | 158 | H- | | | | | | | L | L | H | Q | hR | A | K | W | I | Q | D | L |
| 166 | 160 | H- | | | | | | | L | L | H | Q | R | A | K | W | I | Q | D | $ |
| 167 | 161 | Ac- | | | | | | | L | L | H | Q | R | A | K | W | I | Q | D | $ |
| 169 | 163 | H- | | | | | | | L | L | H | Q | R | A | K | W | I | Q | D | $ |
| 170 | 164 | H- | | | | | | | L | L | H | Q | R | A | K | W | I | Q | D | $ |
| 171 | 165 | H- | | | | | | | L | L | H | Q | R | A | K | W | I | Q | D | $ |
| 172 | 166 | H- | | | | | | | L | L | H | Q | R | A | K | W | I | Q | D | $ |
| 173 | 167 | H- | | | | | | | L | L | H | Q | R | A | K | W | I | Q | D | $ |
| 174 | 168 | H- | | | | | | | L | L | H | Q | R | A | K | W | I | Q | D | $ |
| 175 | 169 | H- | | | | | | | L | L | H | Q | R | A | K | W | I | Q | D | $ |
| 176 | 170 | H- | | | | | | | L | L | H | Q | R | A | K | W | I | Q | D | $ |
| 177 | 171 | H- | | | | | | | L | L | H | Q | R | A | K | W | I | Q | D | $ |
| 178 | 172 | H- | | | | | | | L | L | H | Q | R | A | K | W | I | Q | D | $ |
| 179 | 173 | H- | | | | | | | L | L | H | Q | R | A | K | W | I | Q | D | $ |
| 180 | 174 | H- | | | | | | | L | L | H | Q | R | A | K | W | I | Q | D | $ |
| 181 | 175 | H- | | | | | | | L | L | H | Q | R | A | K | W | I | Q | D | $ |
| 182 | 176 | H- | | | | | | | L | L | H | Q | R | A | K | W | I | Q | D | $ |
| 183 | 177 | H- | | | | | | | L | L | H | Q | R | A | K | W | I | Q | D | $ |
| 184 | 178 | H- | | | | | | | L | L | H | Q | R | A | K | W | I | Q | D | $ |
| 185 | 179 | H- | | | | | | | L | L | H | Q | R | A | K | W | I | Q | D | $ |
| 186 | 180 | H- | | | | | | | L | L | H | Q | R | A | K | W | I | Q | D | $ |
| 187 | 181 | H- | | | | | | | L | L | H | Q | R | A | K | W | I | Q | D | $ |
| 188 | 182 | H- | | | | | | | L | L | H | Q | R | A | K | W | I | Q | D | $ |
| 189 | 183 | H- | | | | | | | L | L | H | Q | R | A | K | W | I | Q | D | $ |
| 190 | 184 | H- | | | | | | | L | L | H | Q | R | A | K | W | I | Q | D | $ |
| 191 | 185 | H- | | | | | | | L | L | H | Q | R | A | K | W | I | Q | D | $ |
| 192 | 186 | H- | | | | | | | L | L | H | Q | R | A | K | W | I | Q | D | $ |
| 193 | 187 | H- | | | | | | | L | L | H | Q | R | A | K | W | I | Q | D | $ |
| 194 | 188 | H- | | | | | | | L | L | H | Q | R | A | K | W | I | Q | D | $ |
| 195 | 189 | H- | | | | | | | L | L | H | Q | R | A | K | W | I | Q | D | $ |
| 196 | 190 | H- | | | | | | | L | L | H | Q | R | A | K | W | I | Q | D | $ |
| 197 | 191 | H- | | | | | | | L | L | H | Q | R | A | K | W | I | Q | D | $ |
| 198 | 192 | H- | | | | | | | L | L | H | Q | R | A | K | W | I | Q | D | $ |
| 199 | 193 | H- | | | | | | | L | L | H | Q | R | A | K | W | I | Q | D | $ |
| 200 | 194 | H- | | | | | | | L | L | H | Q | R | A | K | W | I | Q | D | $ |
| 201 | 195 | H- | | | | | | | L | L | H | Q | R | A | K | W | I | Q | D | $ |
| 202 | 196 | H- | | | | | | | L | L | H | Q | R | A | K | W | I | Q | D | $ |
| 203 | 197 | H- | | | | | | | L | L | H | Q | R | A | K | W | I | Q | D | $ |
| 204 | 198 | H- | | | | | | | L | L | H | Q | R | A | K | W | I | Q | $ | $ |
| 205 | 199 | H- | | | | | | | L | L | $ | Q | R | A | K | W | I | Q | D | $ |
| 206 | 200 | H- | | | | | | | L | L | $ | A | R | A | K | W | I | E | D | $ |
| 207 | 201 | H- | | | | | | | L | L | $ | A | R | A | K | F | I | Q | D | $ |
| 208 | 202 | H- | | | | | | | L | L | $ | Q | R | A | A | W | I | Q | D | $ |
| 209 | 203 | H- | | | | | | | L | L | $ | Q | R | A | K | W | I | Q | D | $ |
| 210 | 204 | H- | | | | | | | L | L | H | E | R | A | K | W | I | Q | D | $ |
| 211 | 205 | H- | | | | | | | L | L | H | D | R | A | K | W | I | Q | D | $ |
| 222 | 216 | H- | | | | | | | L | L | H | Q | hR | A | K | W | I | Q | D | $ |
| 223 | 217 | H- | | | I | Q | | | L | L | H | Q | R | A | K | W | I | Q | D | $ |
| 228 | 222 | Ac- | | | | | | | L | L | H | Q | hR | A | K | W | I | Q | D | $ |
| 255 | 249 | H- | | | | | | | L | L | H | Q | R | A | K | W | I | Q | D | $ |
| 256 | 250 | H- | | | | | | | L | L | H | Q | R | A | K | W | I | Q | D | $ |
| 257 | 251 | H- | | | | | | | L | L | H | Q | R | A | K | W | I | Q | D | $ |
| 258 | 252 | H- | | | | | | | L | L | H | Q | R | A | Cit | W | I | Q | D | $ |
| 259 | 253 | H- | | | | | | | L | L | H | Q | Cit | A | K | W | I | Q | D | $ |
| 260 | 254 | H- | | | | | | | L | L | H | Q | R | A | K | W | I | Q | D | $ |
| 261 | 255 | H- | | | | | | | L | L | H | Q | R | A | K | W | I | Q | D | $ |
| 272 | 266 | H- | | | | | | | L | L | H | Q | $/ | A | K | W | I | Q | D | $ |
| 273 | 267 | H- | | | | | | | L | L | H | Q | Nle | A | K | W | I | Q | D | $ |
| 274 | 268 | H- | | | | | | | L | L | H | Q | hR | A | K | W | I | Q | D | $ |
| 275 | 269 | H- | | | | | | | L | L | H | Q | hR | A | Kfam | W | I | Q | D | $ |
| 276 | 270 | H- | | | | | | | L | L | H | Q | hR | A | Ktam | W | I | Q | D | $ |
| 277 | 271 | H- | | | | | | | L | L | H | Q | hR | A | K | W | I | Q | D | $ |
| 278 | 272 | H- | | | | | | | L | L | H | Q | hR | A | K | W | I | Q | D | $ |
| 279 | 273 | Ac- | | | | | | | $/ | L | H | E | R | S | K | F | I | Q | D | $ |
| 280 | 274 | Ac- | | | | | | | L | $/ | H | E | R | A | K | F | I | Q | D | $ |
| 281 | 275 | Ac- | | | | | | | L | L | H | $/ | R | A | K | F | I | Q | D | $ |
| 282 | 276 | Ac- | | | | | | | L | L | H | Aib | R | A | K | F | I | Q | D | $ |
| 283 | 277 | Ac- | | | | | | | L | L | H | E | $/ | A | K | F | I | Q | D | $ |
| 284 | 278 | Ac- | | | | | | | L | L | $ | E | Nle | A | K | F | I | Q | D | $ |
| 286 | 280 | Ac- | | | | | | | L | L | H | E | R | A | K | F | I | Q | D | L |
| 287 | 281 | Ac- | | | | | | | L | L | $ | E | R | A | $/ | F | I | Q | D | L |
| 288 | 282 | Ac- | | | | | | | L | L | H | E | R | A | Aib | F | I | Q | D | $ |
| 289 | 283 | Ac- | | | | | | | L | L | H | E | R | A | K | F | $/ | Q | D | $ |
| 290 | 284 | Ac- | | | | | | | L | L | H | E | R | A | K | F | I | Q | D | $ |
| 291 | 285 | Ac- | | | | | | | L | L | H | E | R | A | K | F | I | Q | D | $ |
| 292 | 286 | Ac- | | | | | | | L | L | H | E | R | A | K | F | I | Q | D | $ |
| 293 | 287 | Ac- | | | | | | | L | L | H | E | R | A | K | F | I | Q | D | $ |
| 294 | 288 | Ac- | | | | | | | L | L | H | E | R | A | K | F | I | Q | D | $ |
| 295 | 289 | Ac- | | | | | | | L | L | H | E | R | A | K | F | I | Q | D | $ |
| 296 | 290 | Ac- | | | | | | | L | L | H | E | R | A | K | F | I | Q | D | $ |
| 297 | 291 | Ac- | | | | | | | L | L | H | E | R | A | K | F | I | Q | D | $ |
| 298 | 292 | Ac- | | | | | | | L | L | H | E | R | A | K | F | I | Q | D | $ |
| 299 | 293 | Ac- | | | | | | | L | L | H | E | R | A | K | F | I | Q | D | $ |
| 300 | 294 | Ac- | | | | | | | L | L | H | E | R | A | K | F | I | Q | D | $ |
| 301 | 295 | Ac- | | | | | | | L | L | H | E | $/ | A | K | F | I | Q | D | $ |
| 302 | 296 | Ac- | | | | | | | L | L | H | E | $/ | A | K | F | I | Q | D | $ |
| 305 | 299 | Ac- | | | | | | | L | L | H | E | R | Aib | K | F | I | Q | D | $ |
| 399 | 394 | Ac- | | | | | | | L | L | H | Q | R | Aib | K | W | I | Q | D | $ |
| 400 | 395 | Ac- | | | | | | | L | L | H | Q | L | Aib | K | W | I | Q | D | $ |
| 400 | 595 | Ac- | | | | | | | L | L | H | Q | Nle | Aib | K | W | I | Q | D | $ |
| 400 | 695 | Ac- | | | | | | | L | L | H | Q | K | Aib | K | W | I | Q | D | $ |
| 401 | 396 | Ac- | | | | | | | L | L | H | Q | R | A | K | W | $ | Q | D | L |
| 402 | 397 | Ac- | | | | | | | L | L | H | Q | R | A | K | W | $r8 | Q | D | L |
| 403 | 398 | Ac- | | | | | | | L | L | H | Q | R | A | K | W | $r8 | Q | D | L |
| 404 | 400 | Ac- | | | | | | | L | L | H | E | R | Aib | K | F | I | Q | D | $ |
| 370 | 367 | Ac- | | | | | | | L | L | H | E | R | Aib | K | F | I | Q | D | $ |
| 371 | 368 | Ac- | | | | | | | L | L | H | E | Nle | Aib | K | F | I | Q | D | $ |
| 372 | 369 | Ac- | | | | | | | L | L | H | E | Nle | Aib | K | F | I | A | D | $ |
| 373 | 370 | Ac- | | | | | | | L | L | H | E | Leu | Aib | K | F | I | A | D | $ |
| 374 | 371 | Ac- | | | | | | | L | L | H | E | Ile | Aib | K | F | I | A | D | $ |
| 375 | 372 | Ac- | | | | | | | L | L | H | E | Lys | Aib | K | F | I | A | D | $ |
| 405 | 401 | Ac- | | | | | | | L | L | H | E | Cit | Aib | K | F | I | A | D | $ |
| 406 | 402 | Ac- | | | | | | | L | L | H | E | R | Aib | K | W | I | Q | D | $ |
| 376 | 373 | Ac- | | | | | | | L | L | H | E | R | Aib | K | W | I | Q | D | $ |
| 377 | 374 | Ac- | | | | | | | L | L | H | E | Nle | Aib | K | W | I | Q | D | $ |
| 378 | 375 | Ac- | | | | | | | L | L | H | E | Nle | Aib | K | W | I | A | D | $ |
| 379 | 376 | Ac- | | | | | | | L | L | H | E | Leu | Aib | K | W | I | A | D | $ |
| 380 | 377 | Ac- | | | | | | | L | L | H | E | Ile | Aib | K | W | I | A | D | $ |
| 381 | 378 | Ac- | | | | | | | L | L | H | E | Lys | Aib | K | W | I | A | D | $ |
| 407 | 403 | Ac- | | | | | | | L | L | H | E | Cit | Aib | K | W | I | A | D | $ |
| 408 | 404 | Ac- | | | | | | | L | L | H | E | R | Aib | K | F | I | Q | D | $ |
| 69 | 63 | H- | | | | | | | L | L | H | Q | hR | A | $ | W | I | Q | $ | L |
| 70 | 64 | H- | | | | | | | L | L | H | Q | hR | A | K | $ | I | Q | D | $ |
| 71 | 65 | H- | | | | | | | L | L | H | Q | hR | A | K | W | I | Q | D | $ |
| 72 | 66 | H- | | | | | | | L | L | H | Q | hR | A | $ | W | I | Q | $ | L |
| 73 | 67 | H- | | | | | | | L | L | H | Q | hR | A | K | $ | I | Q | D | $ |
| 109 | 103 | H- | | | | | | | L | L | H | Q | hR | A | S | $ | I | Q | D | $ |
| 110 | 104 | H- | | | | | | | L | L | H | Q | hR | A | S | $ | I | Q | D | $ |
| 111 | 105 | H- | | | | | | | L | L | H | Q | hR | A | K | $ | I | Q | D | $ |
| 112 | 106 | H- | | | | | | | L | L | H | Q | hR | A | K | $ | I | Q | D | $ |
| 113 | 107 | H- | | | | | | | L | L | H | Q | hR | A | K | $ | I | Q | D | $ |
| 114 | 108 | H- | | | | | | | L | L | H | Q | hR | A | K | $ | I | Q | D | $ |
| 115 | 109 | H- | | | | | | | L | L | H | Q | hR | A | K | $ | I | Q | D | $ |
| 116 | 110 | H- | | | | | | | L | L | H | Q | hR | A | K | $ | I | Q | D | $ |
| 99 | 93 | H- | | | | | | | L | L | H | Q | hR | A | K | W | I | $ | D | L |
| 100 | 94 | H- | | | | | | | L | L | H | Q | hR | A | K | $ | I | Q | D | $ |
| 101 | 95 | H- | | | | | | | F4Cl | L | H | Q | hR | A | K | $ | I | Q | D | $ |
| 102 | 96 | H- | | | | | | | L | Nle | H | Q | hR | A | K | $ | I | Q | D | $ |
| 103 | 97 | H- | | | | | | | L | K | H | Q | R | A | K | $ | I | Q | D | $ |
| 105 | 99 | H- | | | | | | | F | Nle | H | Q | hR | A | K | $ | I | Q | D | $ |
| 107 | 101 | H- | | | | | | | L | L | H | A | hR | A | K | $ | I | Q | D | $ |
| 108 | 102 | H- | | | | | | | L | L | H | D | hR | A | K | $ | I | Q | D | $ |
| 106 | 100 | H- | | | | | | | L | L | H | Q | hR | A | K | $ | I | Q | D | $ |
| 94 | 88 | H- | | | | | | | L | L | H | $ | hR | A | K | $ | I | Q | D | L |
| 95 | 89 | H- | | | | | | | L | L | $ | Q | hR | A | $ | W | I | Q | D | L |
| 96 | 90 | H- | | | | | | | L | $ | H | Q | hR | $ | K | W | I | Q | D | L |
| 97 | 91 | H- | | | | | | | $ | L | H | Q | $ | A | K | W | I | Q | D | L |
| 85 | 79 | H- | | | | | | | L | L | H | Q | hR | A | K | $ | I | Q | D | $ |
| 89 | 83 | H- | | | | | | | L | L | H | Q | hR | A | K | $ | I | Q | D | $ |
| 87 | 81 | H- | | | | | | | L | L | H | Q | hR | A | K | $ | I | Q | D | $ |
| 86 | 80 | H- | | | | | | | L | L | H | Q | hR | A | K | $ | I | Q | D | $ |
| 74 | 68 | H- | | | | | | | L | L | H | Q | hR | A | K | $ | I | Q | D | $ |
| 92 | 86 | H- | | | | | | | L | L | H | Q | hR | A | $ | W | I | Q | $ | L |
| 93 | 87 | H- | | | | | | | L | L | H | Q | $ | A | K | W | $ | Q | D | L |
| 152 | 146 | H- | | | | | | | F | L | H | Q | hR | A | K | $ | I | Q | D | $ |
| 153 | 147 | H- | | | | | | | F4Cl | L | H | Q | hR | A | K | $ | I | Q | D | $ |
| 154 | 148 | H- | | | | | | | L | Nle | H | Q | hR | A | K | $ | I | Q | D | $ |
| 155 | 149 | H- | | | | | | | L | L | H | Q | R | A | K | $ | I | Q | D | $ |
| 157 | 151 | H- | | | | | | | F | Nle | H | Q | hR | A | K | $ | I | Q | D | $ |
| 158 | 152 | H- | | | | | | | L | L | H | A | hR | A | K | $ | I | Q | D | $ |
| 159 | 153 | H- | | | | | | | L | L | H | D | hR | A | K | $ | I | Q | D | $ |
| 160 | 154 | H- | | | | | | | L | L | H | Q | hR | A | $ | $ | I | Q | D | $ |
| 161 | 155 | H- | | | | | | | L | $ | H | Q | hR | $ | K | W | I | Q | D | L |
| 162 | 156 | H- | | | | | | | $ | L | H | Q | $ | A | K | W | I | Q | D | L |
| 221 | 215 | H- | | | | | | | L | L | H | Q | R | A | K | $ | I | Q | D | $ |
| 227 | 221 | Ac- | | | | | | | L | L | H | Q | R | A | K | $ | I | Q | D | $ |
| 229 | 223 | H- | | | | | | | L | L | H | Q | R | A | K | $ | I | Q | D | $ |
| 230 | 224 | H- | | | | | | | L | L | H | Q | R | A | $ | W | I | Q | $ | L |
| 231 | 225 | H- | | | | | | | L | L | H | Q | R | A | K | $ | I | Q | D | $ |
| 232 | 226 | H- | | | | | | | L | L | H | $ | R | A | K | $ | I | Q | D | L |
| 233 | 227 | H- | | | | | | | L | L | H | $ | R | A | K | $ | I | Q | D | L |
| 234 | 228 | H- | | | | | | | L | L | $ | Q | R | A | $ | W | I | Q | D | L |
| 235 | 229 | H- | | | | | | | L | L | $ | Q | R | A | $ | W | I | Q | D | L |
| 236 | 230 | H- | | | | | | | L | L | H | Q | R | A | K | W | $ | Q | D | L |
| 237 | 231 | H- | | | | | | | L | L | H | Q | R | A | K | W | I | Q | D | $ |
| 238 | 232 | H- | | | | | | | L | L | $ | Q | R | A | $ | W | I | Q | D | $ |
| 239 | 233 | H- | | | | | | | L | L | $ | Q | R | A | $ | W | I | Q | D | $ |
| 254 | 248 | H- | | | | | | | L | L | H | Q | hR | A | K | $ | I | Q | D | $ |
| 308 | 302 | Ac- | | | | | | | L | L | $ | Q | R | A | $ | W | I | Q | D | $ |
| 309 | 303 | Ac- | | | | | | | L | L | $ | Q | R | A | $ | W | I | Q | D | L |
| 310 | 304 | Ac- | | | | | | | L | L | $ | Q | R | A | $ | W | I | Q | D | $ |
| 311 | 305 | Ac- | | | | | | | L | L | $ | Q | R | A | $ | W | I | Q | D | L |
| 312 | 306 | Ac- | | | | | | | L | L | $ | Q | R | A | $ | W | I | Q | D | $ |
| 313 | 307 | Ac- | | | | | | | L | L | $ | Q | R | A | $ | W | I | Q | D | L |
| 314 | 308 | Ac- | | | | | | | L | L | $ | A | R | A | $ | W | I | Q | D | $ |
| 315 | 309 | Ac- | | | | | | | L | L | $ | A | R | A | $ | W | I | Q | D | L |
| 316 | 310 | Ac- | | | | | | | L | L | $ | Q | R | A | $ | W | I | A | D | $ |
| 317 | 311 | Ac- | | | | | | | L | L | $ | Q | R | A | $ | W | I | A | D | L |
| 318 | 312 | Ac- | | | | | | | L | L | $ | A | R | A | $ | W | I | A | D | $ |
| 319 | 313 | Ac- | | | | | | | L | L | $ | A | R | A | $ | Q | I | A | D | L |
| 320 | 314 | Ac- | | | | | | | L | L | $ | Q | R | A | $ | Q | I | A | D | $ |
| 321 | 315 | Ac- | | | | | | | L | L | $ | Q | R | A | $ | Q | I | A | D | L |
| 322 | 316 | Ac- | | | | | | | L | L | $ | A | R | A | $ | Q | I | A | D | $ |
| 347 | 341 | Ac- | | | | | | | L | L | $ | A | R | A | $ | Q | I | A | D | L |
| 365 | 359 | Ac- | | | | | | | L | L | $ | Q | R | A | $ | Q | I | Q | D | L |
| 367 | 361 | Ac- | | | | | | | L | L | $ | Q | R | A | $ | Q | I | Q | D | $ |
| 369 | 363 | Ac- | | | | | | | L | L | H | $ | R | A | K | $ | I | Q | D | L |
| 435 | 431 | Ac- | | | | | | | L | L | $ | Q | R | A | $ | W | I | Q | D | $ |
| 435 | 531 | Ac- | | | | | | | L | L | $ | Q | R | A | $ | W | I | Q | D | $ |
| 409 | 405 | Ac- | | | | | | | L | L | H | Q | R | A | K | $ | I | Q | D | $ |
| 410 | 406 | Ac- | | | | | | | L | L | H | Q | R | A | K | W | I | Q | D | L |
| 411 | 407 | Ac- | | | | | | | L | L | H | $ | R | A | K | $ | I | Q | D | L |
| 412 | 408 | Ac- | | | | | | | L | L | $ | Q | R | A | $ | W | I | Q | D | $ |
| 413 | 409 | Ac- | | | | | | | L | L | H | $ | L | A | K | $ | I | Q | D | L |
| 413 | 509 | Ac- | | | | | | | L | L | H | $ | Nle | A | K | $ | I | Q | D | L |
| 413 | 609 | Ac- | | | | | | | L | L | H | $ | K | A | K | $ | I | Q | D | L |
| 414 | 410 | Ac- | | | | | | | L | L | $ | Q | L | A | $ | W | I | Q | D | L |
| 414 | 510 | Ac- | | | | | | | L | L | $ | Q | Nle | A | $ | W | I | Q | D | L |
| 414 | 610 | Ac- | | | | | | | L | L | $ | Q | K | A | $ | W | I | Q | D | L |
| 415 | 411 | Ac- | | | | | | | L | L | $ | Q | L | A | $ | W | I | Q | D | $ |
| 415 | 511 | Ac- | | | | | | | L | L | $ | Q | Nle | A | $ | W | I | Q | D | $ |
| 415 | 611 | Ac- | | | | | | | L | L | $ | Q | K | A | $ | W | I | Q | D | $ |
| 240 | 234 | H- | | | | | | | L | L | H | Q | R | A | K | W | I | Q | D | $5a5 |
| 241 | 235 | H- | | | | | | | L | L | H | Q | R | A | K | W | I | Q | D | $5n3 |
| 242 | 236 | H- | | | | | | | L | L | H | Q | R | A | K | $5a5 | I | Q | D | $5n3 |
| 243 | 237 | H- | | | | | | | L | L | H | Q | R | A | K | $5n3 | I | Q | D | $5a5 |
| 244 | 238 | H- | | | | | | | L | L | H | Q | R | A | K | $5a5 | I | Q | D | $5n3 |
| 245 | 239 | H- | | | | | | | L | L | H | Q | R | A | $5a5 | W | I | Q | $5n3 | L |
| 216 | 210 | H- | | | | | | | L | L | H | Q | hR | A | K | W | I | Q | D | $r8 |
| 125 | 119 | H- | | | | | | | L | L | H | Q | hR | A | K | W | $r8 | Q | D | L |
| 217 | 211 | H- | | | | | | | L | L | H | Q | hR | A | K | W | I | Q | $r8 | L |
| 127 | 121 | H- | | | | | | | L | L | H | Q | hR | A | K | W | I | Q | $r8 | L |
| 126 | 120 | H- | | | | | | | L | L | H | Q | hR | A | K | $r8 | I | Q | D | L |
| 123 | 117 | H- | | | | | | | L | L | H | Q | hR | A | K | $ | I | Q | D | St |
| 122 | 116 | H- | | | | | | | L | L | H | Q | hR | A | K | W | I | Q | D | $ |
| 215 | 209 | H- | | | | | | | L | L | H | Q | hR | A | K | W | I | Q | D | $r5 |
| SEQ | | | | | | | | | | | | | | | | | | | | |
| ID | SP# | 19 | 20 | 21 | 22 | 23 | 24 | 25 | 26 | 27 | 28 | 29 | 30 | 31 | 32 | 33 | 34 | 35 | 36 | 37 |
| 6 | PTH | E | R | V | E | W | L | R | K | L | L | Q | D | V | H | N | F | | | -NH₂ |
| 7 | PTHRP | R | R | R | F | F | L | H | H | L | I | A | E | I | H | T | A | E | Y | -NH₂ |
| 83 | 77 | R | R | R | F | W | L | H | H | L | I | A | E | I | H | T | A | E | Y | -NH₂ |
| 98 | 92 | R | R | R | $ | W | L | H | $ | L | I | A | E | I | H | T | A | E | Y | -NH₂ |
| 84 | 78 | R | R | R | $ | W | L | H | $ | L | I | A | E | I | H | T | A | E | Y | -NH₂ |
| 91 | 85 | R | R | R | F | W | L | $r8 | H | L | I | A | E | I | $ | T | A | E | Y | -NH₂ |
| 90 | 84 | R | R | R | F | W | L | $r8 | H | L | I | A | E | I | $ | T | A | E | Y | -NH₂ |
| 124 | 118 | R | R | R | $ | W | L | H | H | L | I | A | E | I | H | T | A | E | Y | -NH₂ |
| 163 | 157 | R | R | R | $ | W | L | H | $ | L | I | A | E | I | H | T | A | E | Y | -NH₂ |
| 167 | 158 | R | R | R | $ | W | L | H | $ | L | I | A | E | I | H | T | A | E | Y | -NH₂ |
| 166 | 160 | R | R | R | $ | F | L | H | H | L | I | A | E | I | H | T | A | | | -NH₂ |
| 167 | 161 | R | R | R | $ | F | L | H | H | L | I | A | E | I | H | T | A | | | -NH₂ |
| 169 | 163 | R | R | R | $ | F | L | H | H | L | I | A | E | I | H | F | A | | | -NH₂ |
| 170 | 164 | R | R | R | $ | F | L | H | H | L | I | A | E | I | F | T | A | | | -NH₂ |
| 171 | 165 | R | R | R | $ | F | L | H | H | L | I | A | E | I | A | T | A | | | -NH₂ |
| 172 | 166 | R | R | R | $ | F | L | H | H | L | I | A | E | Nle | H | T | A | | | -NH₂ |
| 173 | 167 | R | R | R | $ | F | L | H | H | L | I | A | E | T | H | T | A | | | -NH₂ |
| 174 | 168 | R | R | R | $ | F | L | H | H | L | I | A | E | Cba | H | T | A | | | -NH₂ |
| 175 | 169 | R | R | R | $ | F | L | H | H | L | I | A | E | Cpg | H | T | A | | | -NH₂ |
| 176 | 170 | R | R | R | $ | F | L | H | HK | L | I | A | A | I | H | T | A | | | -NH₂ |
| 177 | 171 | R | R | R | $ | F | L | H | H | L | I | E | E | I | H | T | A | | | -NH₂ |
| 178 | 172 | R | R | R | $ | F | L | H | H | L | I | S | E | I | H | T | A | | | -NH₂ |
| 179 | 173 | R | R | R | $ | F | L | H | H | L | L | A | E | I | H | T | A | | | -NH₂ |
| 180 | 174 | R | R | R | $ | F | L | H | H | L | Cba | A | E | I | H | T | A | | | -NH₂ |
| 181 | 175 | R | R | R | $ | F | L | H | H | L | Cha | A | E | I | H | T | A | | | -NH₂ |
| 182 | 176 | R | R | R | $ | F | L | H | H | L | CpG | A | E | I | H | T | A | | | -NH₂ |
| 183 | 177 | R | R | R | $ | F | L | H | H | F | I | A | E | I | H | T | A | | | -NH₂ |
| 184 | 178 | R | R | R | $ | F | L | H | H | Nle | I | A | E | I | H | T | A | | | -NH₂ |
| 185 | 179 | R | R | R | $ | F | L | H | H | Y | I | A | E | I | H | T | A | | | -NH₂ |
| 186 | 180 | R | R | R | $ | F | L | H | H | H | I | A | E | I | H | T | A | | | -NH₂ |
| 187 | 181 | R | R | R | $ | F | L | H | H | hF | I | A | E | I | H | T | A | | | -NH₂ |
| 188 | 182 | R | R | R | $ | F | L | H | A | L | I | A | E | I | H | T | A | | | -NH₂ |
| 189 | 183 | R | R | R | $ | F | L | H | F | L | I | A | E | I | H | T | A | | | -NH₂ |
| 190 | 184 | R | R | R | $ | F | L | H | S | L | I | A | E | I | H | T | A | | | -NH₂ |
| 191 | 185 | R | R | R | $ | F | L | H | E | L | I | A | E | I | H | T | A | | | -NH₂ |
| 192 | 186 | R | R | R | $ | F | L | A | H | L | I | A | E | I | H | T | A | | | -NH₂ |
| 193 | 187 | R | R | R | $ | F | L | F | H | L | I | A | E | I | H | T | A | | | -NH₂ |
| 194 | 188 | R | R | R | $ | F | L | S | H | L | I | A | E | I | H | T | A | | | -NH₂ |
| 195 | 189 | R | R | R | $ | F | L | E | H | L | I | A | D | V | H | T | A | | | -NH₂ |
| 196 | 190 | R | R | R | $ | F | Cba | H | H | L | I | A | D | V | H | T | A | | | -NH₂ |
| 197 | 191 | R | R | R | $ | F | Cpg | H | H | L | I | A | D | V | H | T | A | | | -NH₂ |
| 198 | 192 | R | R | R | $ | W | L | H | H | L | I | A | E | I | H | T | A | | | -NH₂ |
| 199 | 193 | R | R | R | $ | 1Nal | L | H | H | L | I | A | E | I | H | T | A | | | -NH₂ |
| 200 | 194 | R | R | R | $ | 2Nal | L | H | H | L | I | A | E | I | H | T | A | | | -NH₂ |
| 201 | 195 | R | R | R | $ | F | L | H | H | L | I | A | E | I | H | T | A | | | -NH₂ |
| 202 | 196 | S | R | R | $ | F | L | H | H | L | I | A | E | I | H | T | A | | | -NH₂ |
| 203 | 197 | E | R | R | $ | F | L | H | H | L | I | A | E | I | H | T | A | | | -NH₂ |
| 204 | 198 | A | R | R | $ | F | L | H | H | L | I | A | E | I | H | T | A | | | -NH₂ |
| 205 | 199 | R | R | R | $ | F | L | H | H | L | I | A | E | I | H | T | A | | | -NH₂ |
| 206 | 200 | R | R | R | $ | F | L | H | H | L | I | A | E | I | H | T | A | | | -NH₂ |
| 207 | 201 | R | R | R | $ | F | L | H | H | L | I | A | E | I | H | T | A | | | -NH₂ |
| 208 | 202 | R | R | R | $ | F | L | H | H | L | I | A | E | I | H | T | A | | | -NH₂ |
| 209 | 203 | R | R | R | $ | F | L | H | H | L | I | A | E | I | H | T | A | | | -NH₂ |
| 210 | 204 | R | R | R | $ | F | L | H | H | L | I | A | E | I | H | T | A | | | -NH₂ |
| 211 | 205 | R | R | R | $ | F | L | H | H | L | I | A | E | I | H | T | A | | | -NH₂ |
| 222 | 216 | R | R | R | $ | F | L | H | H | L | I | A | E | I | H | T | A | | | -NH₂ |
| 223 | 217 | R | R | R | $ | F | L | H | H | L | I | A | E | I | H | T | A | | | -NH₂ |
| 228 | 222 | R | R | R | $ | F | L | H | H | L | I | A | E | I | H | T | A | | | -NH₂ |
| 255 | 249 | R | R | Cit | $ | F | L | H | H | L | I | A | E | I | H | T | A | | | -NH₂ |
| 256 | 250 | R | Cit | R | $ | F | L | H | H | L | I | A | E | I | H | T | A | | | -NH₂ |
| 257 | 251 | Cit | R | R | $ | F | L | H | H | L | I | A | E | I | H | T | A | | | -NH₂ |
| 258 | 252 | R | R | R | $ | F | L | H | H | L | I | A | E | I | H | T | A | | | -NH₂ |
| 259 | 253 | R | R | R | $ | F | L | H | H | L | I | A | E | I | H | T | A | | | -NH₂ |
| 260 | 254 | R | R | R | $ | F | L | H | H | L | I | A | E | I | H | T | A | | | -NH₂ |
| 261 | 255 | R | R | R | $ | F | L | H | H | L | I | A | E | I | H | T | A | | | -NH₂ |
| 272 | 266 | R | R | R | $ | F | L | H | H | L | I | A | E | I | H | T | A | | | -NH₂ |
| 273 | 267 | R | R | R | $ | F | L | H | H | L | I | A | E | I | H | T | A | | | -NH₂ |
| 274 | 268 | R | A | R | $ | A | A | H | H | L | I | A | E | I | H | T | A | E | Y | -NH₂ |
| 275 | 269 | R | R | R | $ | F | L | H | H | L | I | A | E | I | H | T | A | E | Y | -NH₂ |
| 276 | 270 | R | R | R | $ | F | L | H | H | L | I | A | E | I | H | T | A | E | Y | -NH₂ |
| 277 | 271 | R | R | R | $ | F | L | H | H | L | I | A | E | I | H | T | A | E | Y | -NH₂ |
| 278 | 272 | R | R | R | $ | F | L | H | H | L | I | A | E | I | H | T | A | E | Y | -NH₂ |
| 279 | 273 | R | R | R | $ | F | L | H | H | L | I | A | E | I | H | T | A | | | -NH₂ |
| 280 | 274 | R | R | R | $ | F | L | H | H | L | I | A | E | I | H | T | A | | | -NH₂ |
| 281 | 275 | R | R | R | $ | F | L | H | H | L | I | A | E | I | H | T | A | | | -NH₂ |
| 282 | 276 | R | R | R | $ | F | L | H | H | L | I | A | E | I | H | T | A | | | -NH₂ |
| 283 | 277 | R | R | R | $ | F | L | H | H | L | I | A | E | I | H | T | A | | | -NH₂ |
| 284 | 278 | R | R | R | $ | F | L | H | H | L | I | A | E | I | H | T | A | | | -NH₂ |
| 286 | 280 | R | R | R | $ | F | L | H | H | L | I | A | E | I | H | T | A | | | -NH₂ |
| 287 | 281 | R | R | R | $ | F | L | H | H | L | I | A | E | I | H | T | A | | | -NH₂ |
| 288 | 282 | R | R | R | $ | F | L | H | H | L | I | A | E | I | H | T | A | | | -NH₂ |
| 289 | 283 | R | R | R | $ | F | L | H | H | L | I | A | E | I | H | T | A | | | -NH₂ |
| 290 | 284 | R | R | R | $ | F | / | H | H | L | I | A | E | I | H | T | A | | | -NH₂ |
| 291 | 285 | R | R | R | $ | F | L | $/ | H | L | I | A | E | I | H | T | A | | | -NH₂ |
| 292 | 286 | R | R | R | $ | F | L | Aib | H | L | I | A | E | I | H | T | A | | | -NH₂ |
| 293 | 287 | R | R | R | $ | F | L | H | $/ | L | I | A | E | I | H | T | A | | | -NH₂ |
| 294 | 288 | R | R | R | $ | F | L | H | Aib | L | I | A | E | I | H | T | A | | | -NH₂ |
| 295 | 289 | R | R | R | $ | F | L | H | H | $/ | I | A | E | I | H | T | A | | | -NH₂ |
| 296 | 290 | R | R | R | $ | F | L | H | H | L | $/ | A | E | I | H | T | A | | | -NH₂ |
| 297 | 291 | R | R | R | $ | F | L | H | H | L | I | $/ | E | I | H | T | A | | | -NH₂ |
| 298 | 292 | R | R | R | $ | F | L | H | H | L | I | Aib | E | I | H | T | A | | | -NH₂ |
| 299 | 293 | R | R | R | $ | F | L | H | H | L | I | A | E | $/ | T | A | | | | |
| 300 | 294 | R | R | R | $ | F | L | H | H | L | I | A | E | H | T | A | | | | |
| 301 | 295 | R | R | R | $ | F | L | H | H | L | I | $/ | E | H | T | A | | | | |
| 302 | 296 | R | R | R | $ | F | L | H | H | L | $/ | A | E | H | T | A | | | | |
| 305 | 299 | R | R | R | $ | F | L | H | H | L | I | Aib | E | H | T | A | | | | |
| 399 | 394 | R | R | R | $ | W | L | H | K | L | L | Aib | E | H | T | A | | | | |
| 400 | 395 | R | R | R | $ | W | L | H | K | L | L | Aib | E | H | T | A | | | | |
| 400 | 595 | R | R | R | $ | W | L | H | K | L | L | Aib | E | H | T | A | | | | |
| 400 | 695 | R | R | R | $ | W | L | H | K | L | L | Aib | E | H | T | A | | | | |
| 401 | 396 | R | R | R | $r8 | W | L | H | H | L | I | A | E | H | T | A | | | | |
| 402 | 397 | R | R | R | $ | W | L | H | H | L | I | A | E | H | T | A | | | | |
| 403 | 398 | R | R | R | $ | F | L | H | H | L | I | A | E | H | T | A | | | | |
| 404 | 400 | R | R | R | $ | F | L | H | H | L | L | Aib | E | H | T | A | | | | |
| 370 | 367 | R | R | R | $ | F | L | A | H | L | L | Aib | E | H | T | A | | | | |
| 371 | 368 | R | R | R | $ | F | L | A | H | L | L | Aib | E | H | T | A | | | | |
| 372 | 369 | R | R | R | $ | F | L | A | H | L | L | Aib | E | H | T | A | | | | |
| 373 | 370 | R | R | R | $ | F | L | A | H | L | L | Aib | E | I | H | T | A | | | |
| 374 | 371 | R | R | R | $ | F | L | A | H | L | L | Aib | E | I | H | T | A | | | |
| 375 | 372 | R | R | R | $ | F | L | A | H | L | L | Aib | E | I | H | T | A | | | |
| 405 | 401 | R | R | R | $ | F | L | A | H | L | L | Aib | E | I | H | T | A | | | |
| 406 | 402 | R | R | R | $ | W | L | H | H | L | L | Aib | E | I | H | T | A | | | |
| 376 | 373 | R | R | R | $ | W | L | A | H | L | L | Aib | E | I | H | T | A | | | |
| 377 | 374 | R | R | R | $ | W | L | A | H | L | L | Aib | E | I | H | T | A | | | |
| 378 | 375 | R | R | R | $ | W | L | A | H | L | L | Aib | E | I | H | T | A | | | |
| 379 | 376 | R | R | R | $ | W | L | A | H | L | L | Aib | E | I | H | T | A | | | |
| 380 | 377 | R | R | R | $ | W | L | A | H | L | L | Aib | E | I | H | T | A | | | |
| 381 | 378 | R | R | R | $ | W | L | A | H | L | L | Aib | E | I | H | T | A | | | |
| 407 | 403 | R | R | R | $ | W | L | A | H | L | L | Aib | E | I | H | T | A | | | |
| 408 | 404 | R | R | R | $ | F | L | H | H | L | L | Aib | E | I | H | T | A | | | |
| 69 | 63 | R | R | R | F | W | L | H | $ | L | I | A | $ | I | H | T | A | E | Y | |
| 70 | 64 | R | R | R | F | W | L | H | $ | L | I | A | $ | I | H | T | A | E | Y | |
| 71 | 65 | R | R | R | $ | W | L | H | $ | L | I | A | $ | I | H | T | A | E | Y | |
| 72 | 66 | R | R | R | $ | W | L | H | $ | L | I | A | E | I | H | T | A | E | Y | |
| 73 | 67 | R | R | R | $ | W | L | H | $ | L | I | A | E | I | H | T | A | E | Y | |
| 109 | 103 | S | R | Q | $ | W | L | H | $ | Q | I | A | N | I | H | T | A | E | Y | |
| 110 | 104 | S | R | Q | $ | W | L | H | $ | L | I | A | E | I | H | T | A | E | Y | |
| 111 | 105 | R | R | R | $ | W | L | R | $ | F | I | A | E | I | H | T | A | E | Y | |
| 112 | 106 | R | R | R | $ | W | L | R | $ | Y | I | A | E | I | H | T | A | E | Y | |
| 113 | 107 | R | R | R | $ | W | L | W | $ | L | I | A | E | I | H | T | A | E | Y | |
| 114 | 108 | R | R | R | $ | W | L | Y | $ | L | I | A | E | I | H | T | A | E | Y | |
| 115 | 109 | R | R | R | $ | W | L | F | $ | L | I | A | E | I | H | T | A | E | Y | |
| 116 | 110 | R | R | R | $ | W | L | H | $ | L | I | A | E | I | 2Pal | T | A | E | Y | |
| 99 | 93 | R | R | R | $ | W | L | H | $ | L | I | A | E | I | H | T | A | E | Y | |
| 100 | 94 | R | R | R | $ | W | L | H | $ | L | I | A | E | I | H | T | A | E | Y | |
| 101 | 95 | R | R | R | $ | W | L | H | $ | L | I | A | E | I | H | T | A | E | Y | |
| 102 | 96 | R | R | R | $ | W | L | H | $ | L | I | A | E | I | H | T | A | E | Y | |
| 103 | 97 | R | R | R | $ | W | L | H | $ | L | I | A | E | I | H | T | A | E | Y | |
| 105 | 99 | R | R | R | $ | W | L | H | $ | L | I | A | E | I | H | T | A | E | Y | |
| 107 | 101 | R | R | R | $ | W | L | H | $ | L | I | A | E | I | H | T | A | E | Y | |
| 108 | 102 | R | R | R | $ | W | L | H | $ | L | I | A | E | I | H | T | A | E | Y | |
| 106 | 100 | R | R | R | $ | W | L | H | $ | L | I | A | E | I | H | T | A | E | Y | |
| 94 | 88 | R | R | R | $ | W | L | H | $ | L | I | A | E | I | H | T | A | E | Y | |
| 95 | 89 | R | R | R | $ | W | L | H | $ | L | I | A | E | I | H | T | A | E | Y | |
| 96 | 90 | R | R | R | $ | W | L | H | $ | L | I | A | E | I | H | T | A | E | Y | |
| 97 | 91 | R | R | R | $ | W | L | H | $ | L | I | A | E | I | H | T | A | E | Y | |
| 85 | 79 | R | R | R | $ | W | L | H | $ | L | I | A | E | I | H | T | A | | | |
| 89 | 83 | R | R | R | F | W | L | H | H | $ | I | A | E | $ | H | T | A | E | Y | |
| 87 | 81 | R | R | R | F | W | L | $ | H | L | I | $ | E | I | H | T | A | E | Y | |
| 86 | 80 | R | R | R | F | W | $ | H | H | L | $ | A | E | I | H | T | A | E | Y | |
| 74 | 68 | R | R | R | $ | W | L | H | $ | L | I | A | E | I | H | T | A | E | Y | |
| 92 | 86 | R | R | R | F | W | $ | H | H | L | $ | A | E | I | H | T | A | E | Y | |
| 93 | 87 | R | R | R | $ | W | L | H | $ | L | I | A | E | I | H | T | A | E | Y | |
| 152 | 146 | R | R | R | $ | W | L | H | $ | L | I | A | E | I | H | T | A | E | Y | |
| 153 | 147 | R | R | R | $ | W | L | H | $ | L | I | A | E | I | H | T | A | E | Y | |
| 154 | 148 | R | R | R | $ | W | L | H | $ | L | I | A | E | I | H | T | A | E | Y | |
| 155 | 149 | R | R | R | $ | W | L | H | $ | L | I | A | E | I | H | T | A | E | Y | |
| 157 | 151 | R | R | R | $ | W | L | H | $ | L | I | A | E | I | H | T | A | E | Y | |
| 158 | 152 | R | R | R | $ | W | L | H | $ | L | I | A | E | I | H | T | A | E | Y | |
| 159 | 153 | R | R | R | $ | W | L | H | $ | L | I | A | E | I | H | T | A | E | Y | |
| 160 | 154 | R | R | R | $ | W | L | H | $ | L | I | A | E | I | H | T | A | E | Y | |
| 161 | 155 | R | R | R | $ | W | L | H | $ | L | I | A | E | I | H | T | A | E | Y | |
| 162 | 156 | R | R | R | $ | W | L | H | $ | L | I | A | E | I | H | T | A | E | Y | |
| 221 | 215 | R | R | R | F | W | $ | H | H | L | $ | A | E | I | H | T | A | | | |
| 227 | 221 | R | R | R | F | W | $ | H | H | L | $ | A | E | I | H | T | A | | | |
| 229 | 223 | R | R | R | F | W | L | H | H | $ | I | A | E | $ | H | T | A | | | |
| 230 | 224 | R | R | R | F | W | L | H | H | $ | I | A | E | $ | H | T | A | | | |
| 231 | 225 | R | R | $ | F | W | L | $ | H | L | I | A | E | I | H | T | A | | | |
| 232 | 226 | R | R | R | F | W | $ | H | H | L | $ | A | E | I | H | T | A | | | |
| 233 | 227 | R | R | R | F | W | L | H | H | $ | I | A | E | $ | H | T | A | | | |
| 234 | 228 | R | R | R | F | W | $ | H | H | L | $ | A | E | I | H | T | A | | | |
| 235 | 229 | R | R | R | F | W | L | H | H | $ | I | A | E | $ | H | T | A | | | |
| 236 | 230 | $ | R | R | F | W | $ | H | H | L | $ | A | E | I | H | T | A | | | |
| 237 | 231 | R | R | R | $ | W | L | H | H | $ | I | A | E | $ | H | T | A | | | |
| 238 | 232 | R | R | R | $ | W | L | H | H | L | I | A | E | I | H | T | A | | | |
| 239 | 233 | R | R | R | $ | W | L | H | H | $ | I | A | E | $ | H | T | A | | | |
| 254 | 248 | R | A | R | $ | W | L | R | $ | L | I | A | E | I | H | T | A | | | |
| 308 | 302 | R | R | R | $ | W | L | A | H | L | L | A | E | I | H | T | A | | | |
| 309 | 303 | R | R | R | F | W | $ | A | H | L | $ | A | E | I | H | T | A | | | |
| 310 | 304 | R | R | R | $ | W | L | H | A | L | L | A | E | I | H | T | A | | | |
| 311 | 305 | R | R | R | F | W | $ | H | A | L | $ | A | E | I | H | T | A | | | |
| 312 | 306 | R | R | R | $ | W | L | H | K | L | L | A | E | I | H | T | A | | | |
| 313 | 307 | R | R | R | F | W | $ | H | K | L | $ | A | E | I | H | T | A | | | |
| 314 | 308 | R | R | R | $ | W | L | H | H | L | L | A | E | I | H | T | A | | | |
| 315 | 309 | R | R | R | F | W | $ | H | H | L | $ | A | E | I | H | T | A | | | |
| 316 | 310 | R | R | R | $ | W | L | H | H | L | L | A | E | I | H | T | A | | | |
| 317 | 311 | R | R | R | F | W | $ | H | H | L | $ | A | E | I | H | T | A | | | |
| 318 | 312 | R | R | R | $ | W | L | H | H | L | L | A | E | I | H | T | A | | | |
| 319 | 313 | R | R | R | F | W | $ | H | H | L | $ | A | E | I | H | T | A | | | |
| 320 | 314 | R | R | R | $ | W | L | A | H | L | L | A | E | I | H | T | A | | | |
| 321 | 315 | R | R | R | F | W | $ | A | H | L | $ | A | E | I | H | T | A | | | |
| 322 | 316 | R | R | R | $ | W | L | A | H | L | L | A | E | I | H | T | A | | | |
| 347 | 341 | R | R | R | F | W | $ | A | H | L | $ | A | E | I | H | T | A | | | |
| 365 | 359 | R | R | R | F | W | $ | H | H | L | $ | A | E | I | H | T | A | | | |
| 367 | 361 | R | R | R | $ | W | L | H | H | L | I | A | E | I | H | T | A | | | |
| 369 | 363 | R | R | R | F | W | $ | H | H | L | $ | A | E | I | H | T | A | | | |
| 435 | 431 | R | R | R | $ | F | L | H | H | L | I | A | E | I | H | T | A | | | |
| 435 | 531 | R | R | R | $ | F | L | H | K | L | I | A | E | I | H | T | A | | | |
| 409 | 405 | R | R | R | F | W | $ | H | K | L | $ | A | E | I | H | T | A | | | |
| 410 | 406 | $ | R | R | F | W | $ | H | K | L | $ | A | E | I | H | T | A | | | |
| 411 | 407 | R | R | R | F | W | $ | H | K | L | $ | A | E | I | H | T | A | | | |
| 412 | 408 | R | R | R | $ | W | L | H | K | L | L | Aib | E | I | H | T | A | | | |
| 413 | 409 | R | R | R | F | W | $ | H | K | L | $ | A | E | I | H | T | A | | | |
| 413 | 509 | R | R | R | F | W | $ | H | K | L | $ | A | E | I | H | T | A | | | |
| 413 | 609 | R | R | R | F | W | $ | H | K | L | $ | A | E | I | H | T | A | | | |
| 414 | 410 | R | R | R | F | W | $ | H | K | L | $ | A | E | I | H | T | A | | | |
| 414 | 510 | R | R | R | F | W | $ | H | K | L | $ | A | E | I | H | T | A | | | |
| 414 | 610 | R | R | R | F | W | $ | H | K | L | $ | A | E | I | H | T | A | | | |
| 415 | 411 | R | R | R | $ | W | L | H | K | L | L | Aib | E | I | H | T | A | | | |
| 415 | 511 | R | R | R | $ | W | L | H | K | L | L | Aib | E | I | H | T | A | | | |
| 415 | 611 | R | R | R | $ | W | L | H | K | L | L | Aib | E | I | H | T | A | | | |
| 240 | 234 | R | R | R | $5n3 | F | L | H | K | L | I | A | E | I | H | T | A | | | |
| 241 | 235 | R | R | R | $5a5 | F | L | H | K | L | I | A | E | I | H | T | A | | | |
| 242 | 236 | R | R | R | $5n3 | W | L | H | $5a5 | L | I | A | E | I | H | T | A | | | |
| 243 | 237 | R | R | R | $5a5 | W | L | H | $5n3 | L | I | A | E | I | H | T | A | | | |
| 244 | 238 | R | R | R | F | W | $5a5 | H | H | L | $5n3 | A | E | I | H | T | A | | | |
| 245 | 239 | R | R | R | F | W | $5n3 | H | H | L | $5n3 | A | E | I | H | T | A | | | |
| 246 | 210 | R | R | R | F | W | L | $ | H | L | I | A | E | I | H | T | A | E | Y | |
| 125 | 119 | R | R | R | $ | W | L | H | H | L | I | A | E | I | H | T | A | E | Y | |
| 217 | 211 | R | R | R | F | W | $ | H | H | L | I | A | E | I | H | T | A | E | Y | |
| 127 | 121 | R | R | R | F | W | $ | H | H | L | I | A | E | I | H | T | A | E | Y | |
| 126 | 120 | R | R | R | F | W | L | H | H | L | I | A | E | I | H | T | A | E | Y | |
| 123 | 117 | R | R | R | $r5 | W | L | H | H | L | I | A | E | I | H | T | A | E | Y | |
| 122 | 116 | R | R | R | St | W | L | H | $r5 | L | I | A | E | I | H | T | A | E | Y | |
| 215 | 209 | R | R | R | St | W | L | H | $ | L | I | A | E | I | H | T | A | E | Y | |

TABLE 8

| SEQ ID NO | Structures |
|---|---|
| 21 | 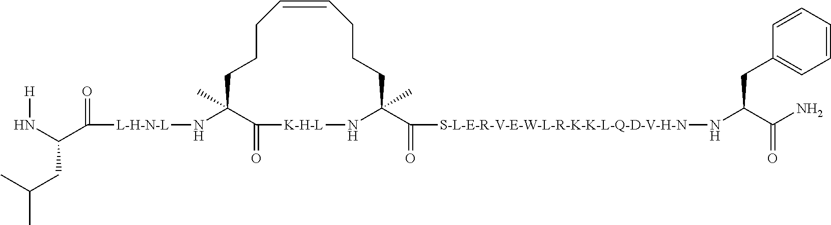 SP# 15 |
| 22 | 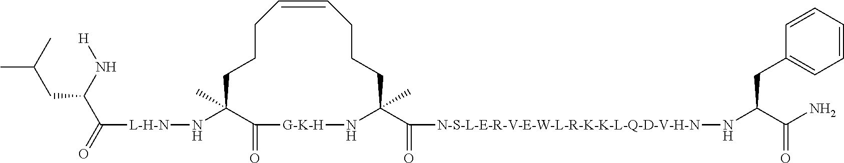 SP# 16 |
| 23 | 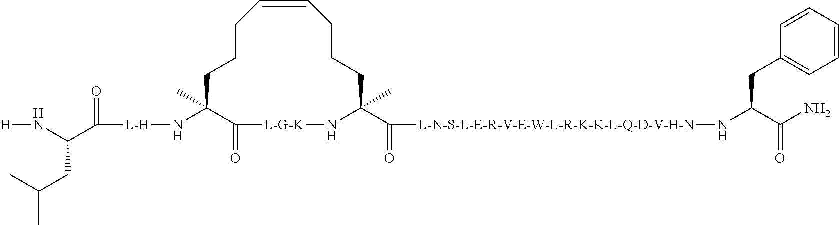 SP# 17 |
| 49 | 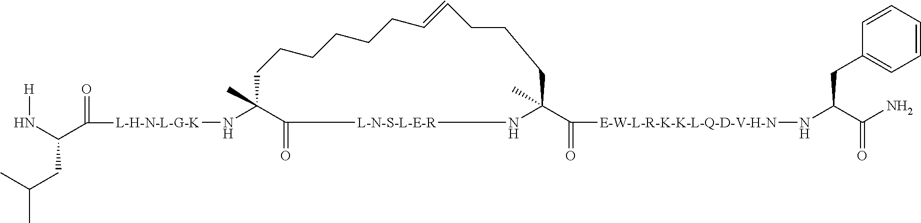 SP# 43 |
| 48 | 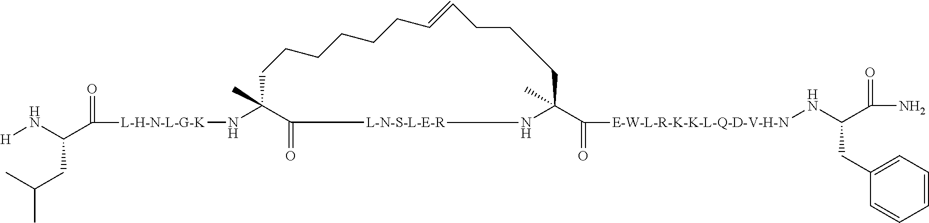 SP# 42 |
| 165 | 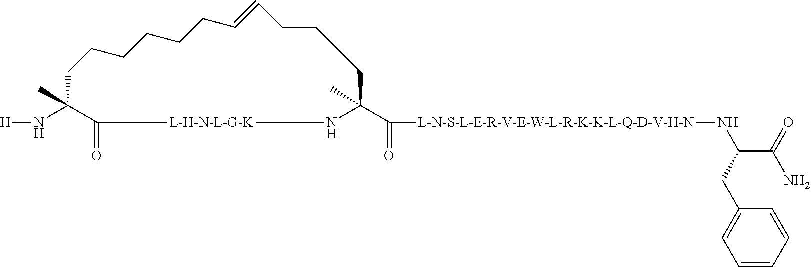 SP# 159 |
| 289 | 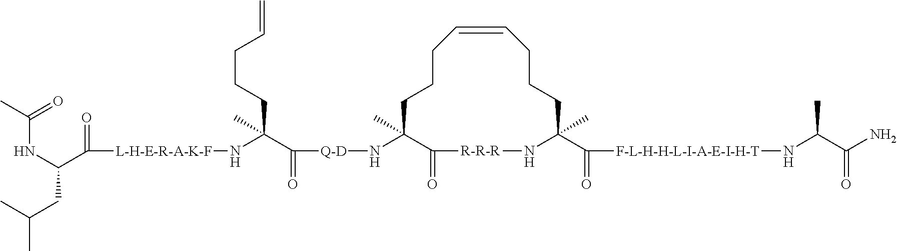 SP# 283 |
| 282 | 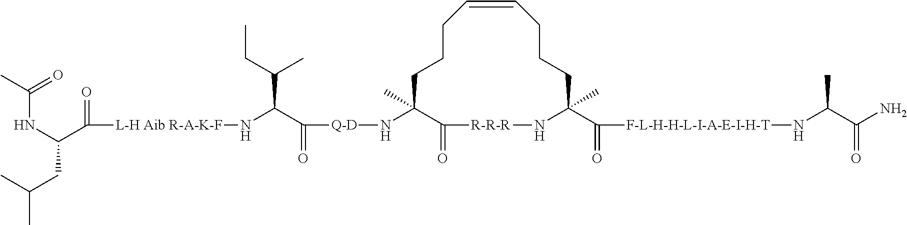 SP# 276 |
| 305 | 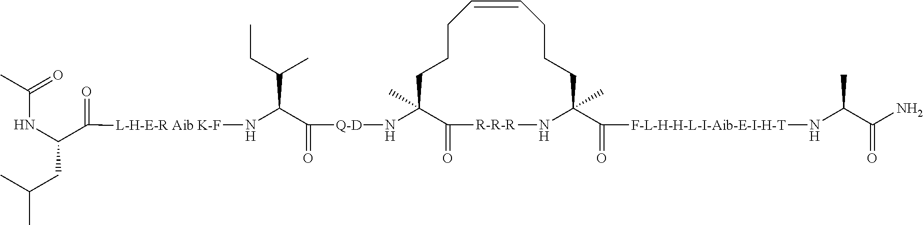 SP# 299 |
| 179 | 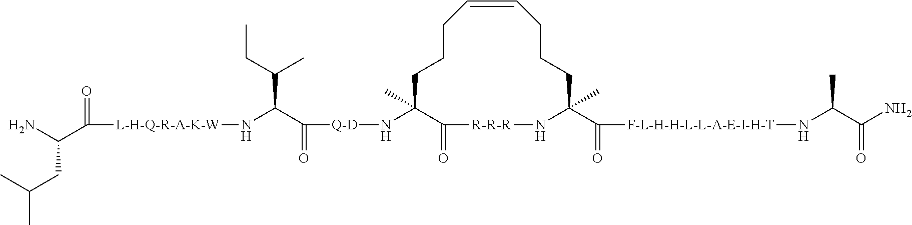 SP# 173 |
| 294 | 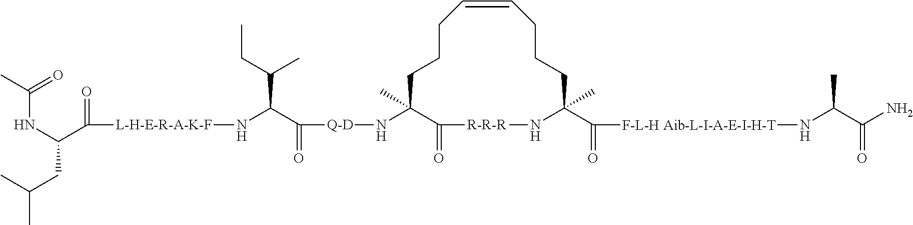 SP# 288 |
| 292 | 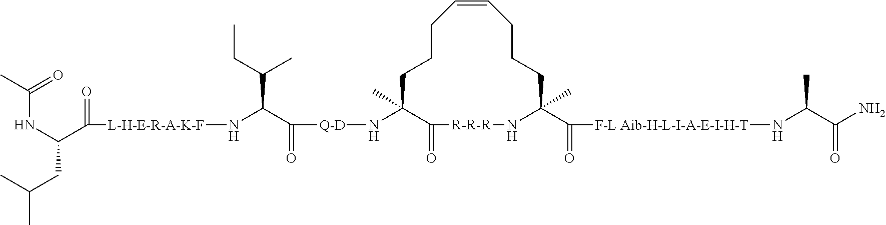 SP# 286 |
| 198 | 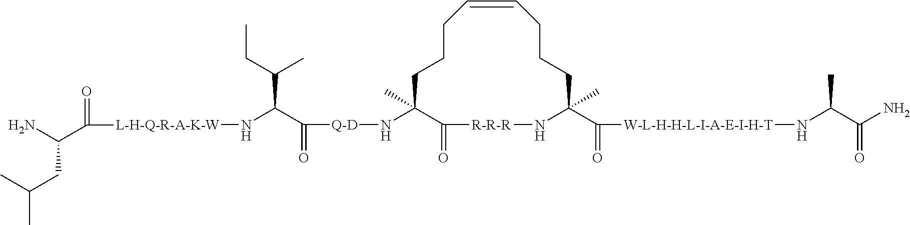 SP# 192 |
| 80 | 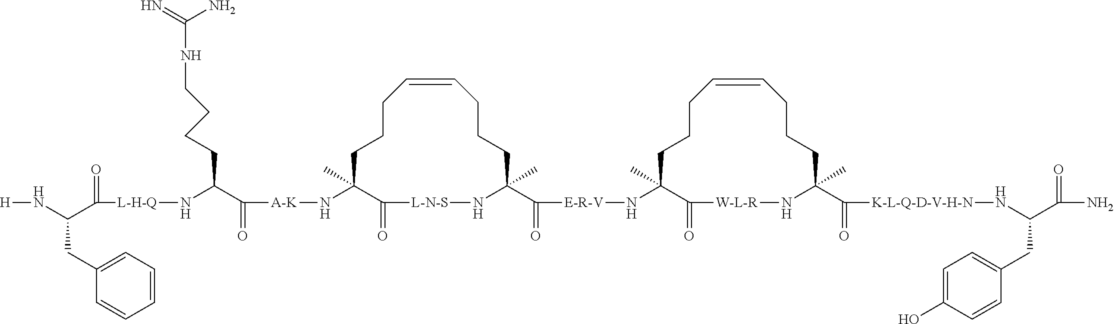 SP# 74 |
| 77 | 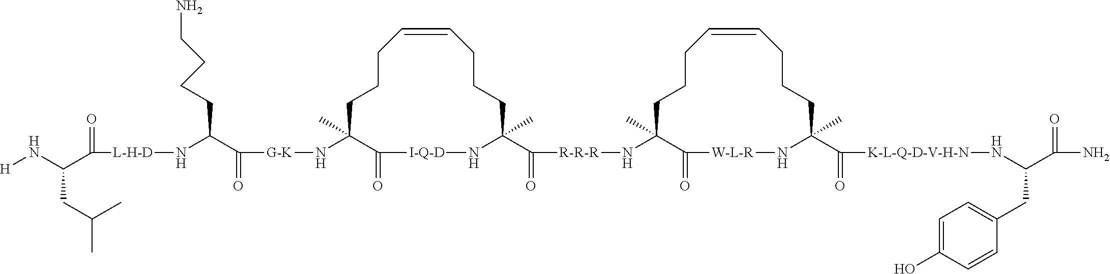 SP# 71 |
| 82 | 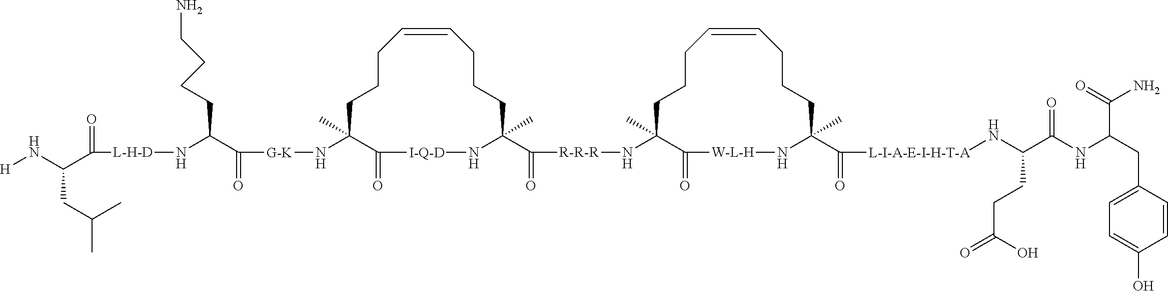 SP# 76 |
| 155 | 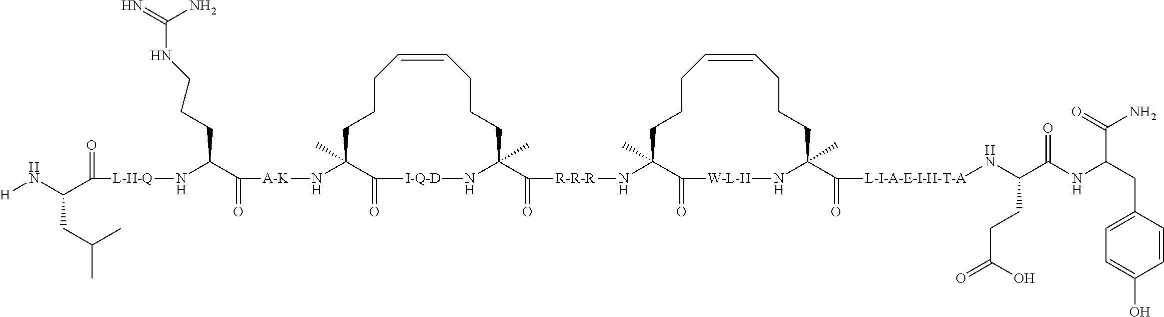 SP# 149 |
| 218 | 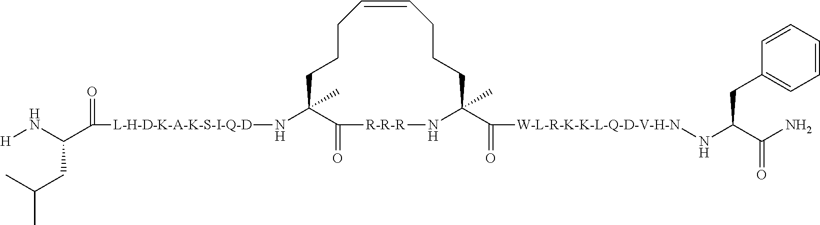 SP# 212 |
| 220 | 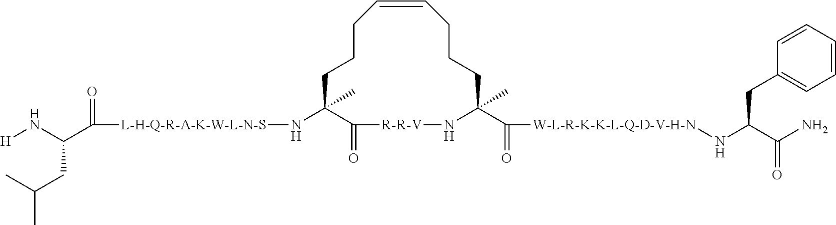 SP# 214 |
| 224 | 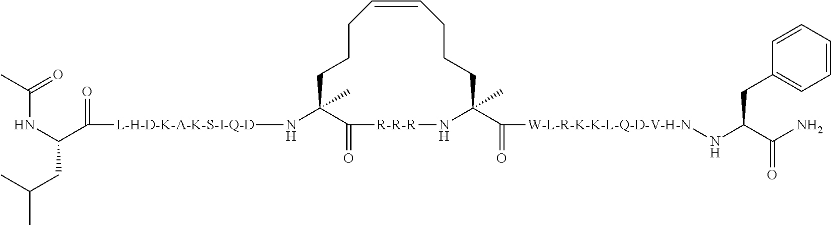 SP# 218 |
| 226 | 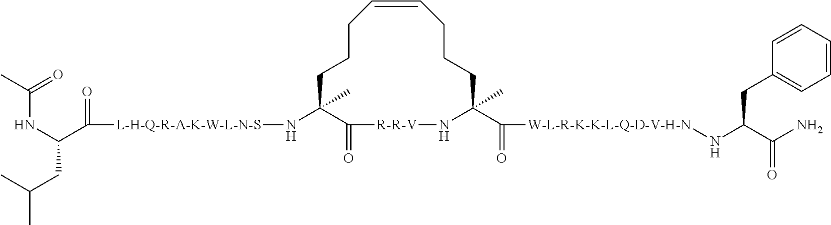 SP# 220 |
| 234 | 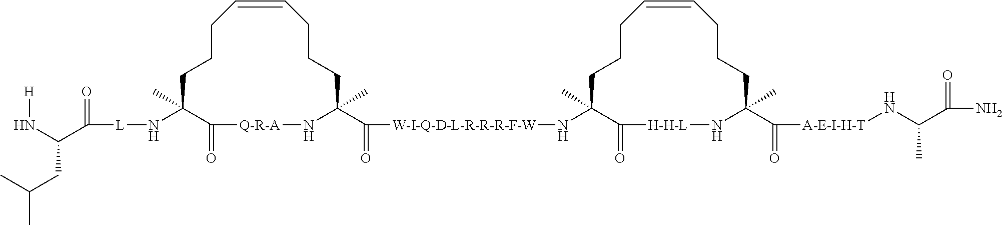 SP# 228 |
| 238 | 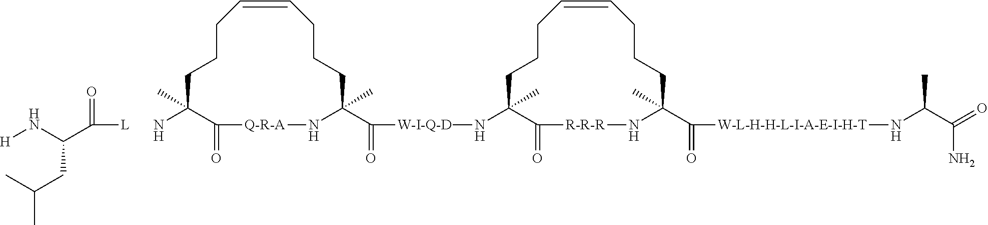 SP# 232 |
| 246 | 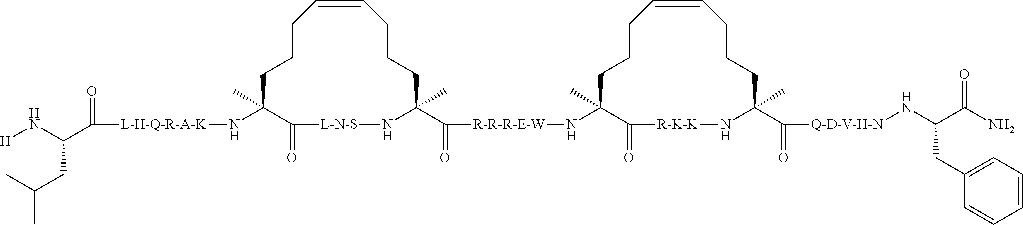 SP# 240 |
| 249 | 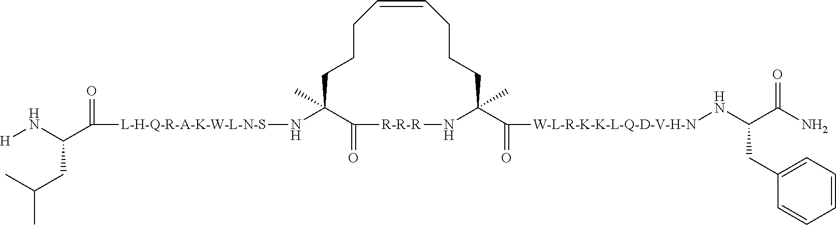 SP# 243 |
| 253 | 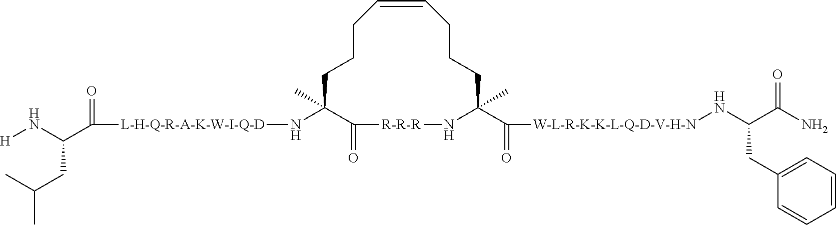 SP# 247 |
| 265 | 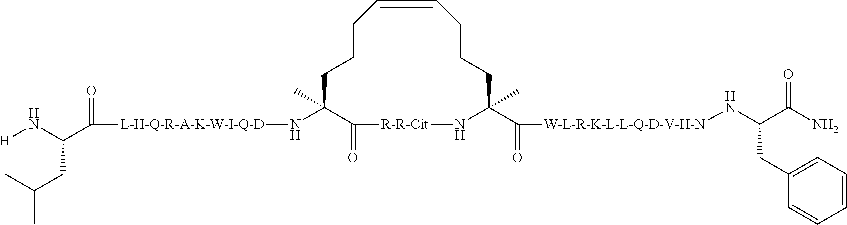 SP# 259 |
| 306 | 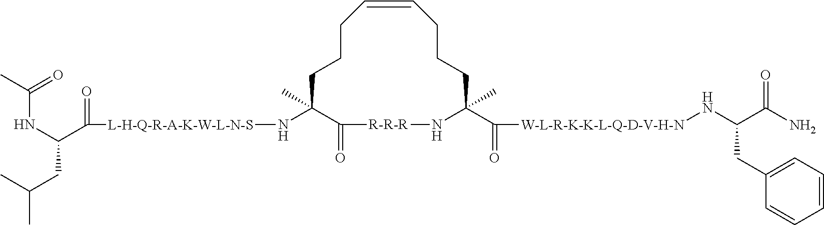 SP# 300 |
| 307 | 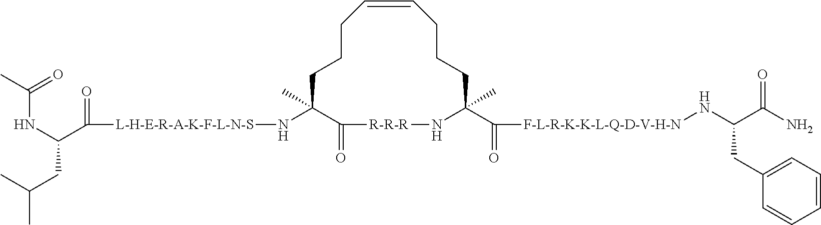 SP# 301 |
| 247 | 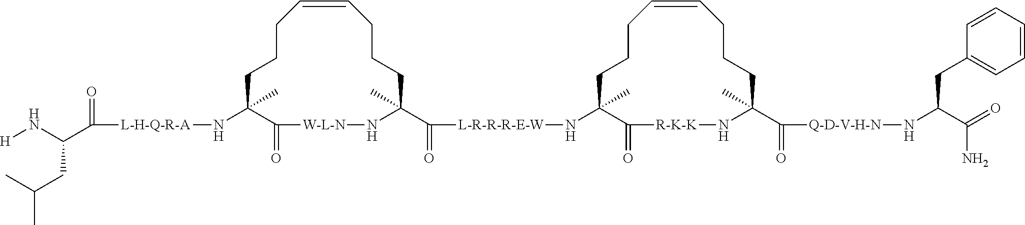 SP# 241 |
| 248 | 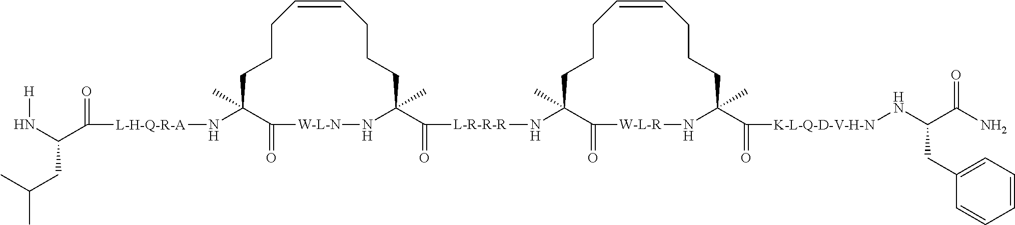 SP# 242 |
| 312 | 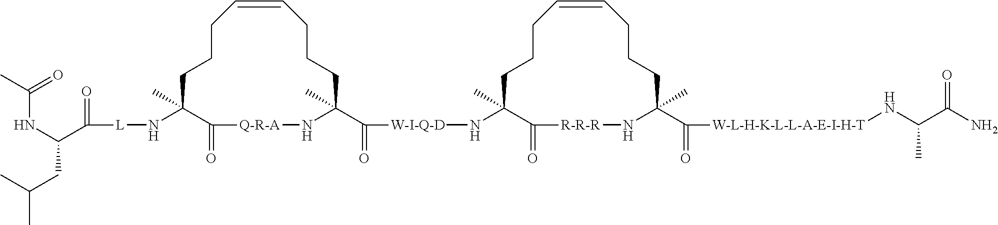 SP# 306 |
| 313 | 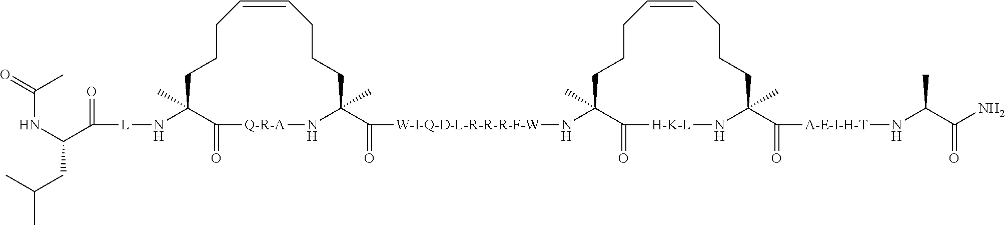 SP# 307 |
| 335 | 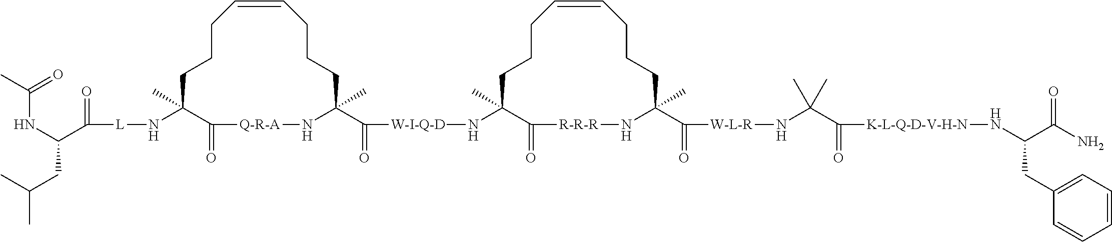 SP# 329 |
| 348 | 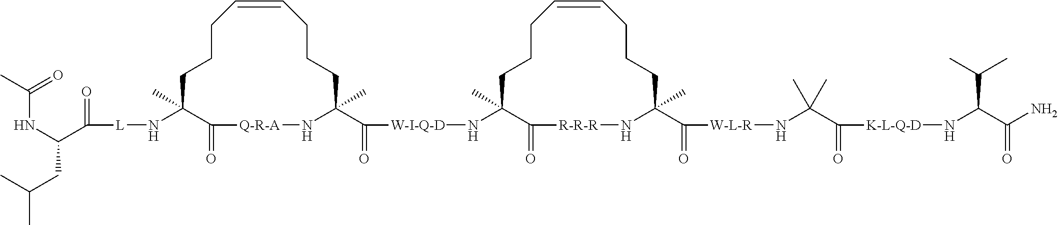 SP# 342 |
| 349 | 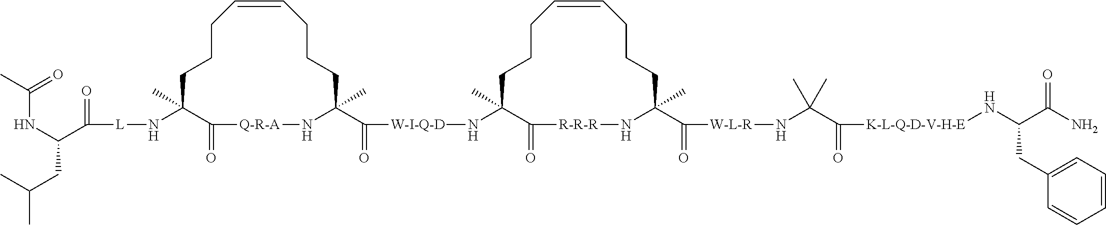 SP# 343 |
| 351 | 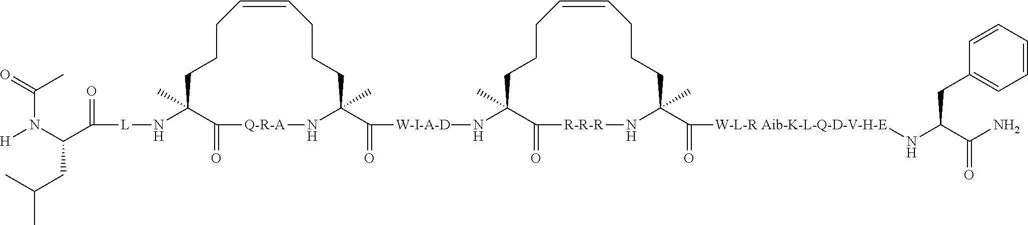 SP# 345 |
| 353 | 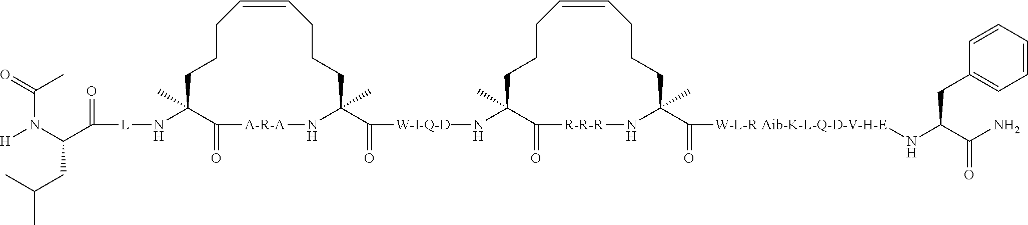 SP# 347 |
| 355 | 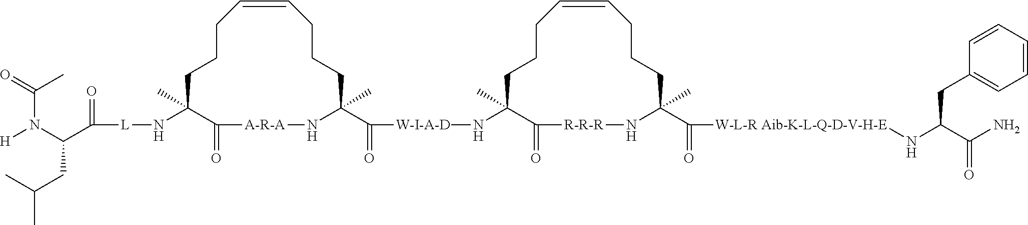 SP# 349 |
| 367 | 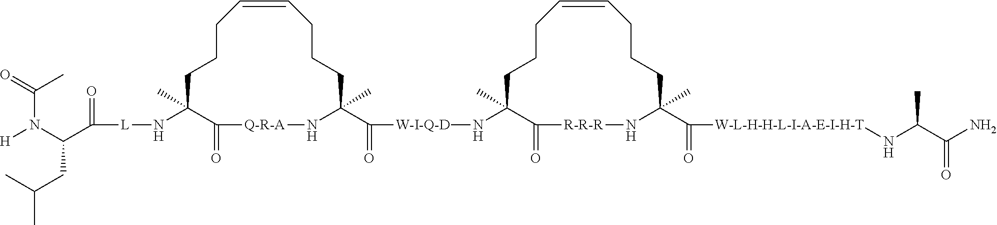 SP# 361 |
| 357 | 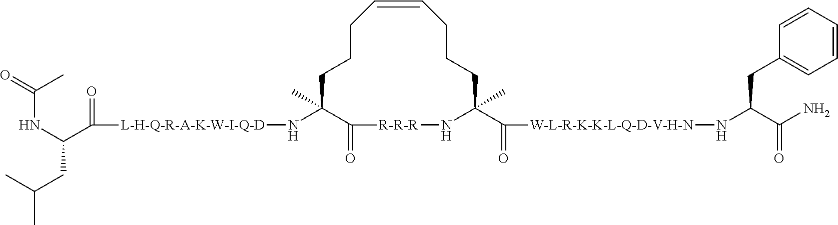 SP# 351 |
| 359 | 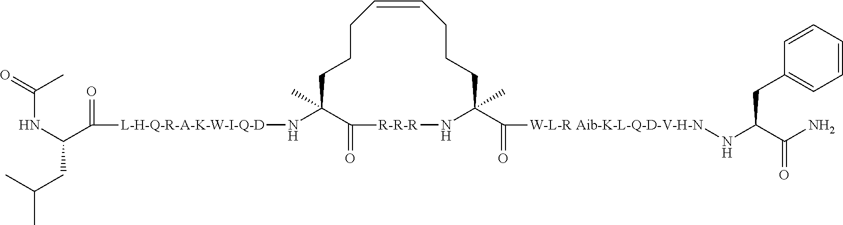 SP# 353 |
| 360 | 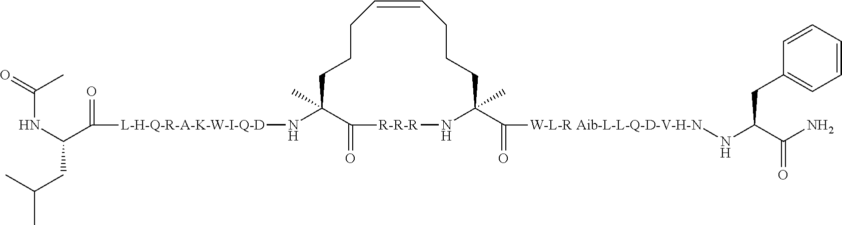 SP# 354 |
| 361 | 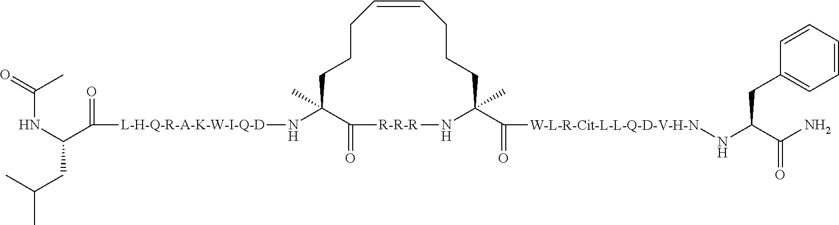 SP# 355 |
| 364 | 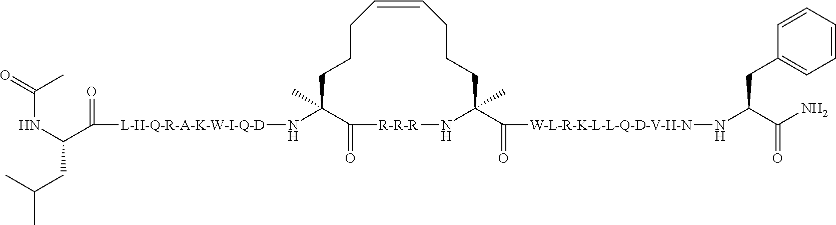 SP# 358 |
| 366 | 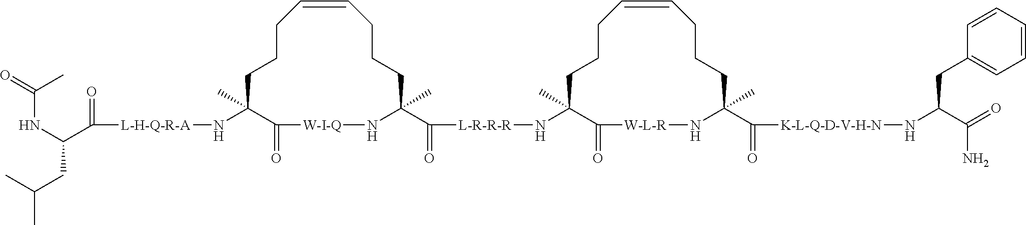 SP# 360 |
| 369 | 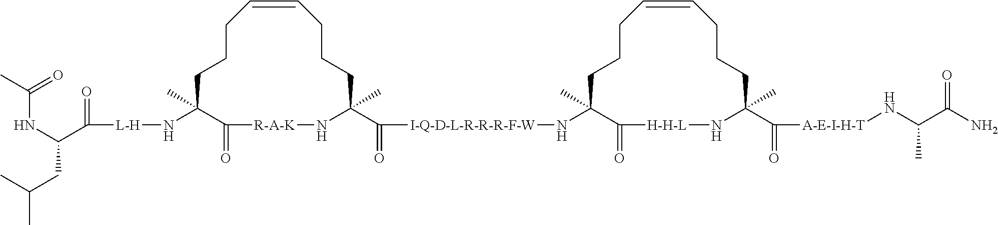 SP# 363 |
| 306 | 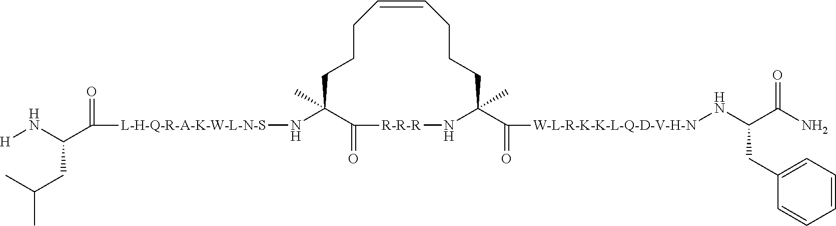 SP# 300 |
| 224 | 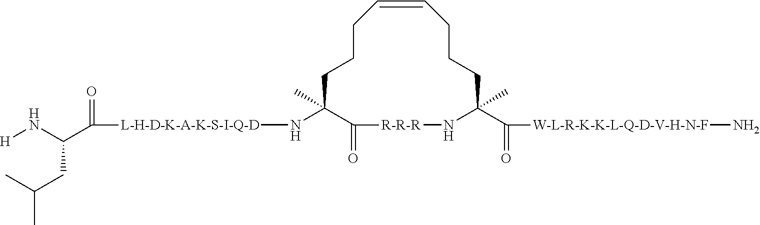 SP# 218 |
| 248 | 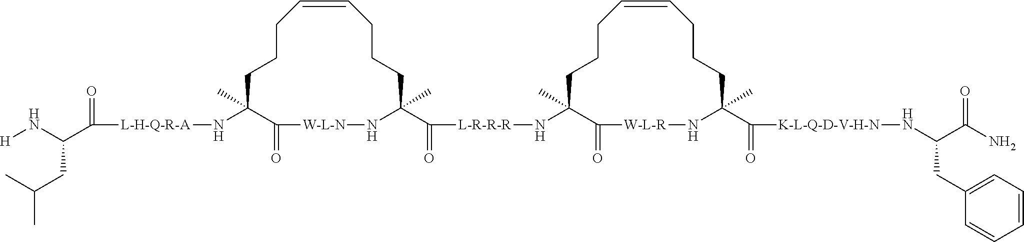 SP# 242 |
| 73 | 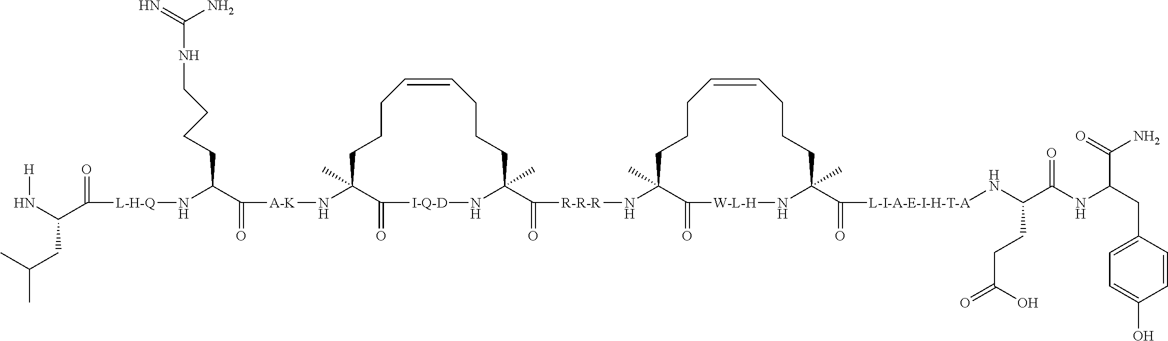 SP# 67 |
| 252 | 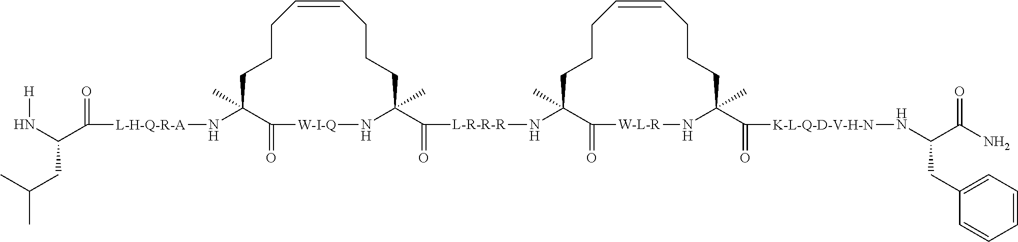 SP# 246 |

In the sequences shown above and elsewhere, the following abbreviations are used: amino acids represented as “$” are alpha-Me S5-pentenyl-alanine olefin amino acids connected by an all-carbon i to i+4 crosslinker comprising one double bond. Amino acids represented as “$r8” are alpha-Me R₈-octenyl-alanine olefin amino acids connected by an all-carbon i to i+7 crosslinker comprising one double bond. “Nle” represents norleucine. “Aib” represents 2-aminoisobutyric acid. “Ac” represents acetyl. Amino acids represented as “Ba” are beta-alanine. Amino acids designated as “Cba” represent cyclobutyl alanine. Amino acids designated as “F4cooh” represent 4-carboxy phenylalanine. Amino acids represented as “$/” are alpha-Me S5-pentenyl-alanine olefin amino acids that are not connected by any crosslinker. “$r5” are alpha-Me R₅-pentenyl-alanine olefin amino acids connected by an all-carbon comprising one double bond. Amino acids represented as “$/r5” are alpha-Me R₅-pentenyl-alanine olefin amino acids that are not connected by any crosslinker. Amino acids represented as “St” are amino acids comprising two pentenyl-alanine olefin side chains, each of which is crosslinked to another amino acid as indicated. Amino acids represented as “StaS” are amino acids comprising two R₅-pentenyl-alanine olefin side chains, each of which is crosslinked to another amino acid as indicated. “hF” represents homophenylalanine. “hR” represents homoarginine. “Pal” represents pyridyl-alanine. “Nal” represents naphtalanine. “Bip” represents 3-biphenyl-4-yl-1-alanine. “Ac5c” represents 1-aminocyclopentane-1-carboxylic acid. “PhAc” represents phenyl acetate. “F4NH₂” represents 4-amino phenylalanine. “F4Cl” represents 4-chloro phenylalanine. The abbreviation “b-” prior to an amino acid represent a beta configuration for the amino acid (e.g., “b-hF” or “b-hPhe” represent beta-phenylalanine, “b-hIle” is beta-homoisoleucine, “b-Ala” is beta-alanine).

“Bpa” represents 4-benzyoyl-phenylalanine; it is a photoreactive amino acid analog useful in making photoreactive stapled peptides that covalently capture their physiologic targets, for example Braun et al. Chem Biol. 2010 Dec. 22; 17(12):1325-33 and Leshchiner et al. Proc Natl Acad Sci USA. 2013 Feb. 12.

Amino acids which are used in the formation of triazole cross-linkers are represented according to the legend indicated below. Stereochemistry at the alpha position of each amino acid is S unless otherwise indicated. For azide amino acids, the number of carbon atoms indicated refers to the number of methylene units between the alpha carbon and the terminal azide. For alkyne amino acids, the number of carbon atoms indicated is the number of methylene units between the alpha position and the triazole moiety plus the two carbon atoms within the triazole group derived from the alkyne.
$5rn3 Alpha-Me R-azide 1,5 triazole (3 carbon)
$5a5Alpha-Me alkyne 1,5 triazole (5 carbon)
$5n3 Alpha-Me azide 1,5 triazole (3 carbon)
$4rn6Alpha-Me R-azide 1,4 triazole (6 carbon)
$4a5Alpha-Me alkyne 1,4 triazole (5 carbon)
   Peptidomimetic Macrocycles Derived from PTH and/or PTHrP

In some embodiments peptidomimetic macrocycles are provided which are derived from PTH. In some embodiments peptidomimetic macrocycles are provided which are derived from PTHrP. In some embodiments peptidomimetic macrocycles are provided which are derived from PTH and PTHrP. In some embodiments, a peptidomimetic macrocycle is provided comprising an amino acid sequence that has at least about 60% sequence identity to an amino acid sequence selected from the group consisting of the amino acid sequences in 1a, 1b, 2a, 2b, 3a, 3b, 5, 6 or 7, wherein the peptidomimetic macrocycle comprises at least one macrocycle-forming linker, wherein the macrocycle-forming linker connects a first amino acid to a second amino acid. In some embodiments, the macrocycle-forming linker does not comprise an amide group. In some embodiments, the peptidomimetic macrocycle comprises an amino acid sequence that has at least about 65%, 70%, 75%, 80%, 85%, 90% 95%, 97%, 98%, 99% or 100% sequence identity to an amino acid sequence selected from the group consisting of the amino acid sequences in 1a, 1b, 2a, 2b, 3a, 3b, 5, 6 or 7. In some embodiments, the peptidomimetic macrocycle comprises an amino acid sequence selected from the group consisting of the amino acid sequences in Table 1a, 1b, 2a, 2b, 3a, 3b, 5, 6 or 7, wherein the peptidomimetic macrocycle comprises at least one macrocycle-forming linker, wherein the macrocycle-forming linker connects a first amino acid to a second amino acid. In some embodiments, the peptidomimetic macrocycle comprises a C-terminal truncation of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids from an amino acid sequence in Table 1a, 1b, 2a, 2b, 3a, 3b, 5, 6 or 7. In some embodiments, the peptidomimetic macrocycle comprises a N-terminal truncation of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids from an amino acid sequence in Table 1a, 1b, 2a, 2b, 3a, 3b, 5, 6 or 7.

In some embodiments, a macrocycle-forming linker of the peptidomimetic macrocycle connects one of the following pairs of amino acids: 1 and 5, 2 and 6, 3 and 7, 4 and 8, 5 and 9, 6 and 10, 7 and 11, 8 and 12, 9 and 13, 10 and 14, 11 and 15, 12 and 16, 13 and 17, 14 and 18, 15 and 19, 17 and 21, 18 and 22, 21 and 25, 22 and 26, 24 and 28, 25 and 29, 26 and 30, 27 and 31, 28 and 32 or 29 and 33.

In some embodiments, a macrocycle-forming linker of the peptidomimetic macrocycle connects one of the following pairs of amino acids: 1 and 8, 2 and 9, 3 and 10, 4 and 11, 5 and 12, 6 and 13, 7 and 14, 8 and 15, 9 and 16, 10 and 17, 11 and 18, 12 and 19, 14 and 21, 15 and 22, 17 and 24, 18 and 25, 19 and 26, 21 and 28, 22 and 29, 24 and 31, 25 and 32, or 26 and 33.

In some embodiments, the macrocycle-forming linker connects amino acids 7 and 11, 7 and 14, 8 and 12, 9 and 13, 10 and 14, 11 and 15, 12 and 16, 13 and 17, 14 and 18, 14 and 21, 15 and 19, 15 and 22, 17 and 24, 18 and 22, 18 and 25, 22 and 26, 22 and 29, 24 and 28, 25 and 32, 26 and 30, 26 and 33, or 27 and 31. For example, the macrocycle-forming linker connects amino acids 7 and 11, 8 and 12, 9 and 13, 10 and 14, 13 and 17, 14 and 18, or 18 and 22.

In some embodiments, a macrocycle-forming linker of the peptidomimetic macrocycle connects one of the following pairs of amino acids: 9 and 13, 10 and 14, 15 and 19, 15 and 22, 16 and 20, 16, and 23, 17 and 21, 17 and 24, 18 and 22, 18 and 25, 19 and 23, 19 and 26, 20 and 24, 20 and 27, 21 and 25, 21, and 28, 22 and 26, 22 and 29, 23 and 27, 23 and 30, 24 and 28, 24 and 31, 25 and 29, 25 and 32, 26 and 30, 26 and 33, 27 and 31, 27 and 34, 28 and 32, 28 and 35, 29 and 33, 29 and 36, 30 and 34, 31 and 35, or 32 and 36.

In some embodiments, the macrocycle-forming linker connects amino acids 14 and 18. In some embodiments, the macrocycle-forming linker connects amino acids 22 and 26. In some embodiments, the macrocycle-forming linker connects amino acids 26 and 30. In some embodiments the peptidomimetic macrocycle comprises two pairs of crosslinked amino acids. In some embodiments, the macrocycle-forming linker connects amino acids 14 and 18 and amino acids 26 and 30. In some embodiments, the macrocycle-forming linker connects amino acids 13 and 17 and amino acids 26 and 30.

In some embodiments, the peptidomimetic macrocycle comprises two pairs of crosslinked amino acids. In some embodiments, a first and second macrocycle-forming linker of the peptidomimetic macrocycle connects two of the following pairs of amino acids: 1 and 5, 2 and 6, 3 and 7, 4 and 8, 5 and 9, 6 and 10, 7 and 11, 8 and 12, 9 and 13, 10 and 14, 11 and 15, 12 and 16, 13 and 17, 14 and 18, 15 and 19, 17 and 21, 18 and 22, 21 and 25, 22 and 26, 24 and 28, 25 and 29, 26 and 30, 27 and 31, 28 and 32, or 29 and 33. In some embodiments, a first and second macrocycle-forming linker of the peptidomimetic macrocycle connects two of the following pairs of amino acids: 1 and 8, 2 and 9, 3 and 10, 4 and 11, 5 and 12, 6 and 13, 7 and 14, 8 and 15, 9 and 16, 10 and 17, 11 and 18, 12 and 19, 14 and 21, 15 and 22, 17 and 24, 18 and 25, 19 and 26, 21 and 28, 22 and 29, 24 and 31, 25 and 32, or 26 and 33.

For example, the first macrocycle-forming linker connects amino acids 7 and 11, 7 and 14, 8 and 12, 9 and 13, 10 and 14, 11 and 15, 12 and 16, 13 and 17, 14 and 18, 14 and 21, 15 and 19, 15 and 22, 17 and 24, 18 and 22, 18 and 25, 22 and 26, 22 and 29, 24 and 28, 25 and 32, 26 and 30, 26 and 33, or 27 and 31, and the second macrocycle-forming linker connects amino acids 18 and 22, 22 and 26, 24 and 28, or 26 and 30.

In some embodiments, the second macrocycle-forming linker connects amino acids 22 and 26. In some embodiments, the second macrocycle-forming linker connects amino acids 24 and 28. In some embodiments, the second macrocycle-forming linker connects amino acids 26 and 30. In some embodiments, the first macrocycle-forming linker connects amino acids 7 and 11 and the second macrocycle-forming linker connects amino acids 22 and 26. In some embodiments, the first macrocycle-forming linker connects amino acids 8 and 12 and the second macrocycle-forming linker connects amino acids 22 and 26. In some embodiments, the first macrocycle-forming linker connects amino acids 13 and 17 and the second macrocycle-forming linker connects amino acids 26 and 30. In some embodiments, the first macrocycle-forming linker connects amino acids 13 and 17, the second macrocycle-forming linker connects amino acids 26 and 30, and the peptidomimetic macrocycle comprises an amino acid substitution at X₁₂. In some embodiments, the first macrocycle-forming linker connects amino acids 14 and 18 and the second macrocycle-forming linker connects amino acids 26 and 30. In some embodiments, the first macrocycle-forming linker connects amino acids 18 and 22 and the second macrocycle-forming linker connects amino acids 26 and 30. In some embodiments, the first macrocycle-forming linker connects amino acids 13 and 17 and the second macrocycle-forming linker connects amino acids 22 and 26. In some embodiments, the first macrocycle-forming linker connects amino acids 14 and 18 and the second macrocycle-forming linker connects amino acids 22 and 26. In some embodiments, the first macrocycle-forming linker connects amino acids 14 and 18 and the second macrocycle-forming linker connects amino acids 24 and 28. In some embodiments, the first macrocycle-forming linker connects amino acids 14 and 18 and the second macrocycle-forming linker connects amino acids 27 and 31.

In some embodiments, the peptidomimetic macrocycle comprises three pairs of crosslinked amino acids. In some embodiments, the first and second macrocycle-forming linkers are as described above and the third macrocycle-forming linker connects amino acids 27 and 31.

In some embodiments, a peptidomimetic macrocycle comprises a helix, for example an α-helix. In some embodiments, a peptidomimetic macrocycle comprises an α,α-disubstituted amino acid. In some embodiments, each amino acid connected by the macrocycle-forming linker is an α,α-disubstituted amino acid.

In some embodiments, the at least one macrocycle-forming linker is a straight chain alkenyl. In some embodiments, the at least one macrocycle-forming linker is a straight chain alkenyl with 6 to 14 carbon atoms. In some embodiments, the at least one macrocycle-forming linker is a straight chain alkenyl with 8 to 12 carbon atoms, for example 8, 9, 10, 11 or 12 carbon atoms. In some embodiments, the at least one macrocycle-forming linker is a C₈ alkenyl with a double bond between C₄ and C₅ of the C₈ alkenyl. In some embodiments, the at least one macrocycle-forming linker is a C₁₂ alkenyl with a double bond between C₄ and C₅ or C₅ and C₆ of the C₁₂ alkenyl.

In some embodiments, the at least one macrocycle-forming linker comprises a first and a second macrocycle-forming linker, wherein the first macrocycle-forming linker connects a first and a second amino acid, wherein the second macrocycle-forming linker connects a third and a fourth amino acid, wherein the first amino acid is upstream of the second amino acid, the second amino acid is upstream of the third amino acid, and the third amino acid is upstream of the fourth amino acid. In some embodiments, 1, 2, 3, 4, 5, 6, or 7, amino acids are between the second and third amino acids. In some embodiments, 4 or 5 amino acids are between the second and third amino acids.

In some embodiments, the at least one macrocycle-forming linker comprises a first and a second macrocycle-forming linker that are separated by 2, 3, 4, 5, 6, or 7 amino acids. In some embodiments, the at least one macrocycle-forming linker comprises a first and a second macrocycle-forming linker that are separated by 4 or 5 amino acids.

In some embodiments, the peptidomimetic macrocycle contains 16-36 amino acids, for example 16, 17, 18, 19, 20, 21, 22, 23, 24, 25, 26, 27, 28, 29, 30, 31, 32, 33, 34, 35 or 36 amino acids. In some embodiments, the peptidomimetic macrocycle contains 24-36 amino acids, for example 24, 25, 26, 27, 28, 29, 30, 31, 32, 33, 34, 35 or 36 amino acids.

Exemplary amino acid substitutions of a peptidomimetic macrocycle provided herein can be seen in Table 4.

In some embodiments, a peptidomimetic macrocycle is provided having the Formula (I):

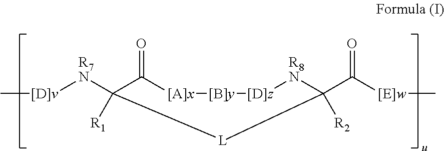

wherein:
each A, C, D, and E is independently an amino acid (including natural or non-natural amino acids and amino acid analogs) and the terminal D and E independently optionally include a capping group,
each B is independently an amino acid (including natural or non-natural amino acids and amino acid analogs),

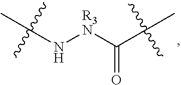

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each R₃ is independently —H, alkyl, alkenyl, alkynyl, arylalkyl, heteroalkyl, cycloalkyl, heterocycloalkyl, cycloalkylalkyl, aryl, or heteroaryl, optionally substituted with R₅; each L and L′ is independently
a macrocycle-forming linker of the formula -L₁-L₂-

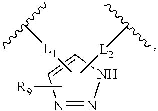

or -L₁-S-L₂-S-L₃-;
each L₁, L₂ and L₃ is independently alkylene, alkenylene, alkynylene, heteroalkylene, cycloalkylene, heterocycloalkylene, arylene, heteroarylene, or [—R₄—K—R₄—]ₙ, each being optionally substituted with R₅; when L is not

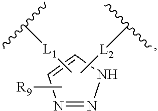

or -L₁-S-L₂-S-L₃-, L₁ and L₂ are alkylene, alkenylene, alkynylene, heteroalkylene, cycloalkylene, heterocycloalkylene, arylene or heteroarylene;
each R₄ is independently alkylene, alkenylene, alkynylene, heteroalkylene, cycloalkylene, heterocycloalkylene, arylene or heteroarylene;
each K is independently O, S, SO, SO₂, CO, CO₂ or CONR₃;
   each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
   each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
   each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
   each R₉ is independently alkyl, alkenyl, alkynyl, aryl, cycloalkyl, cycloalkenyl, heteroaryl, or heterocyclyl group, unsubstituted or optionally substituted with Rₐ and/or R_{b};
   each Rₐ and R_{b} is independently alkyl. OCH₃, CF₃, NH₂, CH₂NH₂, F, Br, I,

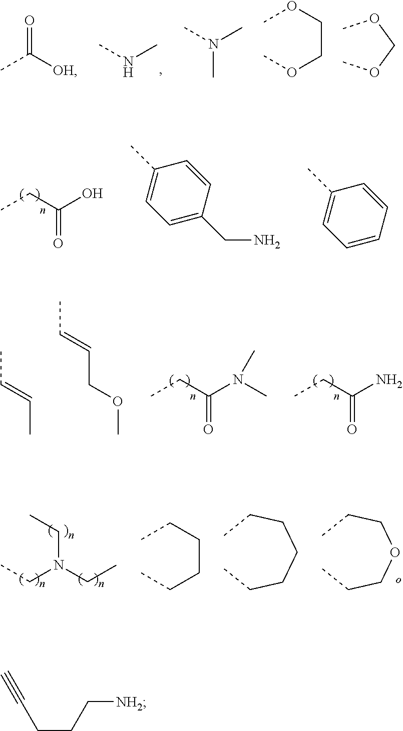

each v and w is independently an integer from 0-1000, for example 0-500, 0-200, 0-100, 0-50, 0-30, 0-20, or 0-10;
u is an integer from 1-10, for example 1-5, 1-3 or 1-2;
each x, y and z is independently an integer from 0-10, for example the sum of x+y+z is 2, 3, or 6;
each n is independently an integer from 1-5; and
and wherein A, B, C, D, and E, taken together with the crosslinked amino acids connected by the macrocycle-forming linker -L₁-L₂-, form an amino acid sequence of the peptidomimetic macrocycle that has at least about 60%, 65%, 70%, 75%, 80%, 85%, 90%, 95%, 96%, 97%, 98%, or 99% sequence identity to a sequence of Table 1a, 1b, 2a, 2b, 3a, 3b, 5, 6 or 7.

In some embodiments, u is 1.

In some embodiments, the sum of x+y+z is 2, 3, 6, or 10, for example 2, 3 or 6, for example 3 or 6. In some embodiments, the sum of x+y+z is 3.

In some embodiments, each of v and w is independently an integer from 1-10, 1-15, 1-20, or 1-25.

In some embodiments, each of v and w is independently an integer from 1-15.

In some embodiments, L₁ and L₂ are independently alkylene, alkenylene or alkynylene. In some embodiments, L₁ and L₂ are independently C₃-C₁₀ alkylene or alkenylene. In some embodiments, L₁ and L₂ are independently C₃-C₆ alkylene or alkenylene.

In some embodiments, L or L′ is:

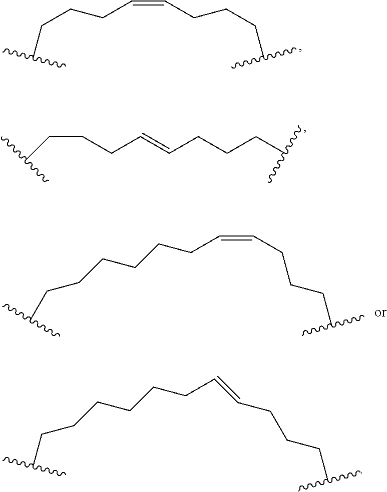 [SMILES: *CCC/C=C\CCC* ; *CCC/C=C/CCC* ; *CCC/C=C\CCCCCC* ; *CCC/C=C/CCCCCC*]

In some embodiments, L or L′ is

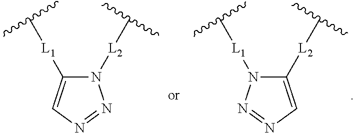

For example, L or L′ is

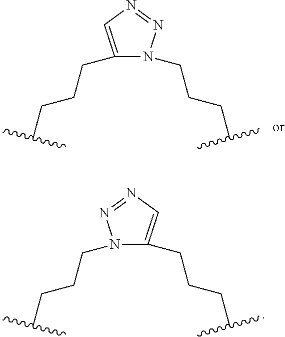 [SMILES: *CCCc1cnnn1CCC* ; *CCCc1cnnn1CCC*]

In some embodiments, R₁ and R₂ are H.

In some embodiments, R₁ and R₂ are independently alkyl.

In some embodiments, R₁ and R₂ are methyl.

In some embodiments, a peptidomimetic macrocycle is provided having the Formula (Ia):

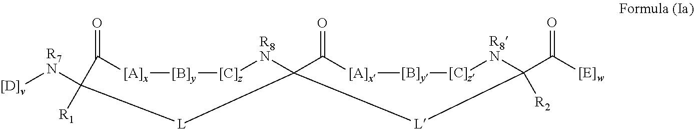

wherein: R₈′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a E residue;
v′ and w′ are independently integers from 0-100; and
x′, y′ and z′ are independently integers from 0-10, e.g., x′+y′+z′ is 2, 3, 6 or 10.

In some embodiments, u is 2.

In some embodiments, a peptidomimetic macrocycle is provided having the Formula (Ib):

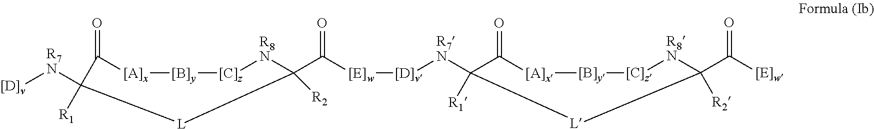

wherein R₇′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
v′ and w′ are independently integers from 0-100; and
x′, y′ and z′ are independently integers from 0-10.

In some embodiments, the sum of x+y+z is 2, 3 or 6, for example 3 or 6.

In some embodiments, the sum of x′+y′+z′ is 2, 3 or 6, for example 3 or 6.

In some embodiments, each of v and w is independently an integer from 1-10, 1-15, 1-20, or 1-25.

In some embodiments, a peptidomimetic macrocycle comprises an amino acid sequence with at least about 60%, 65%, 70%, 75%, 80%, 85%, 90%, 95%, 96%, 97%, 98%, or 99% sequence identity to a sequence of Table 1 or 2, wherein the peptidomimetic macrocycle comprises at least one macrocycle-forming linker, wherein the macrocycle-forming linker connects amino acids 14 and 18.

In some embodiments, a peptidomimetic macrocycle is provided having the Formula (I):

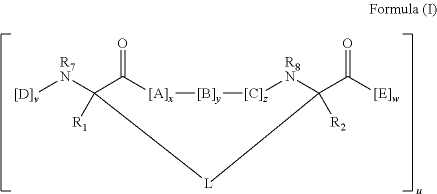

wherein:
each A, C, D, and E is independently an amino acid;
each B is independently an amino acid,

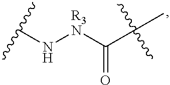

[—NH-L₃-CO—], [—NH-L₃-SO₂—], or [—NH-L₃-];
each R₁ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each R₃ is independently —H, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-,

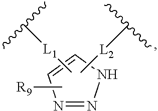

or -L₁-S-L₂-S-L₃-;
each L₁, L₂ and L₃ is independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R₄ is alkylene, alkenylene, alkynylene, heteroalkylene, cycloalkylene, heterocycloalkylene, arylene or heteroarylene;
each K is independently O, S, SO, SO₂, CO, CO₂ or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each R₉ is independently alkyl, alkenyl, alkynyl, aryl, cycloalkyl, cycloalkenyl, heteroaryl, or heterocyclyl group, unsubstituted or optionally substituted with Rₐ and/or Rₐ;
each Rₐ and Rₐ is independently alkyl, OCH₃, CF₃, NH₂, CH₂NH₂, F, Br, I,

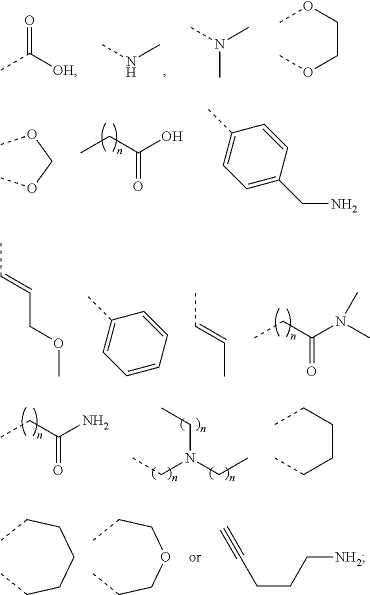

each v and w is independently an integer from 0-1000, for example 0-500, 0-200, 0-100, 0-50, 0-30, 0-20, or 0-10;
u is an integer from 1-10, for example 1-5, 1-3 or 1-2;
each x, y and z is independently an integer from 0-10, for example the sum of x+y+z is 2, 3, or 6; and n is an integer from 1-5.

In other embodiments, a peptidomimetic macrocycle is provided having the Formula (II) or Formula (IIa):

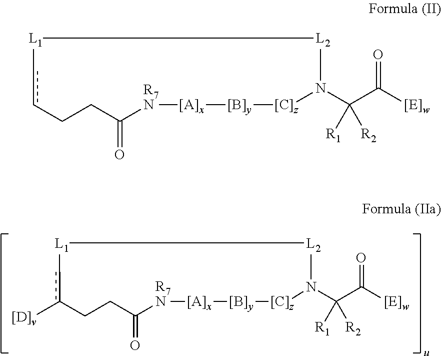

wherein:
each A, C, D, and E is independently a natural or non-natural amino acid, and the terminal D and E independently optionally include a capping group;
each B is independently a natural or non-natural amino acid, amino acid analog,

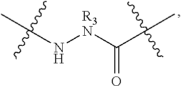

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each R₃ is independently —H, alkyl, alkenyl, alkynyl, arylalkyl, heteroalkyl, cycloalkyl, heterocycloalkyl, cycloalkylalkyl, aryl, or heteroaryl, optionally substituted with R₅;
L is a macrocycle-forming linker of the formula -L₁-L₂-;
each L₁ and L₂ is independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂, or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅;
each v and w is independently an integer from 0-100;
u is an integer from 1-10;
each x, y and z is independently an integer from 0-10;
each n is independently an integer from 1-5; and
A, B, C, and E, taken together with the crosslinked amino acids connected by the macrocycle-forming linker -L₁-L₂-, form an amino acid sequence of the peptidomimetic macrocycle that has at least about 60%, 65%, 70%, 75%, 80%, 85%, 90%, 95%, 96%, 97%, 98%, 99% or 100% sequence identity to a sequence of Table 1a, 1b, 2a, 2b, 3a, 3b, 5, 6 or 7.

In some embodiments, a peptidomimetic macrocycle comprises Formula (IIIa) or Formula (IIIb):

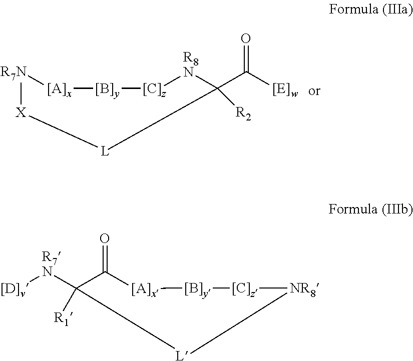

wherein:
each A, C, D and E is independently an amino acid, and the terminal D and E independently optionally include a capping group;
each B is independently an amino acid,

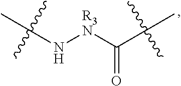

[—NH-L₃-CO—], [—NH-L₃-SO₂—], or [—NH-L₃-]; each R₁′ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or R₂ forms a macrocycle-forming linker L′ connected to the alpha position of one of said E amino acids;
R₃ is —H, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-,

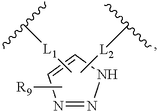

or -L₁-S-L₂-S-L₃-;
each L₁, L₂ and L₃ is independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R₄ is independently alkylene, alkenylene, alkynylene, heteroalkylene, cycloalkylene, heterocycloalkylene, arylene, or heteroarylene;
each K independently is O, S, SO, SO₂, CO, CO₂ or CONR₃;
each R₅ independently is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ or R₇′ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈ or R₈′ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
R₉ is alkyl, alkenyl, alkynyl, aryl, cycloalkyl, cycloalkenyl, heteroaryl, or heterocyclyl group, unsubstituted or optionally substituted with Rₐ and/or R_{b};
Rₐ and R_{b} are independently alkyl, OCH₃, CF₃, NH₂, CH₂NH₂, F, Br, I,

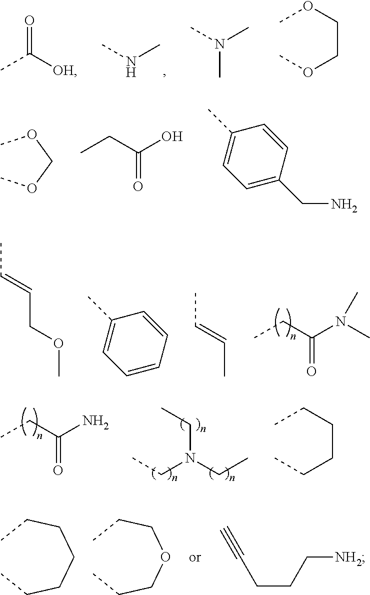

v and w′ are independently integers from 0-1000, for example 0-500, 0-200, 0-100, 0-50, 0-30, 0-20, or 0-10;
x, y, z, x′, y′ and z′ are independently integers from 0-10, for example the sum of x+y+z is 2, 3, 6 or 10, or the sum of x′+y′+z′ is 2, 3, 6, or 10;
n is an integer from 1-5;

### X is C═O, CHR_{c}, or C═S;

R_{c} is alkyl, alkenyl, alkynyl, arylalkyl, cycloalkyl, cycloalkylalkyl, heteroalkyl, or heterocycloalkyl; and A, B, C, and E, taken together with the crosslinked amino acids connected by the macrocycle-forming linker -L₁-L₂-, form an amino acid sequence of the peptidomimetic macrocycle that has at least about 60%, 65%, 70%, 75%, 80%, 85%, 90%, 95%, 96%, 97%, 98%, 99%, or 100% sequence identity to a sequence of Table 1a, 1b, 2a, 2b, 3a, 3b, 5, 6 or 7.

In some embodiments, the peptidomimetic macrocycle has the Formula:

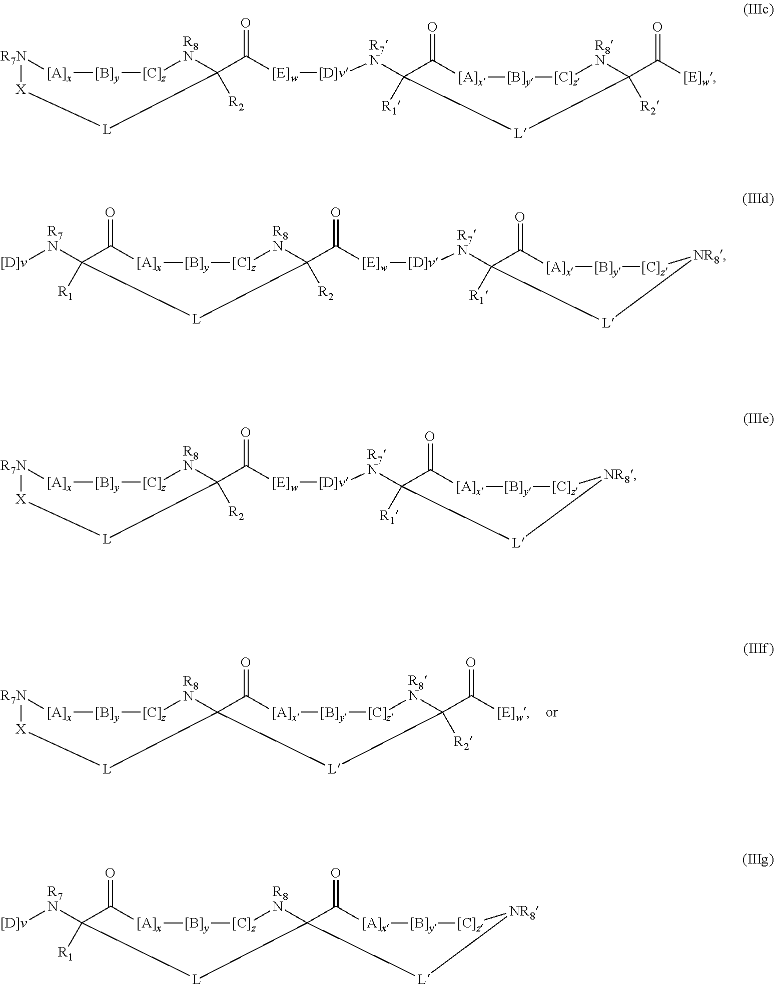

wherein
each R₁′ or R₂′ is independently —H, alkyl, alkenyl, alkynyl, arylalkyl, cycloalkyl, cycloalkylalkyl, heteroalkyl, or heterocycloalkyl, unsubstituted or substituted with halo-; and
v, w, v′ or w′ is independently an integer from 0-100.

In some embodiments, the notation “Hep” is used for a macrocycle of Formula (IIIa), which represents an N-terminal heptenoic capping group of the following formula:

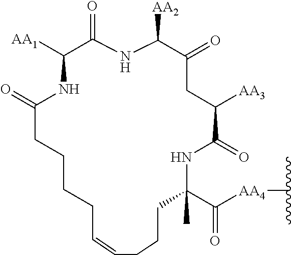

wherein AA₁, AA, AA₃ and AA₄ are amino acids.

In other embodiments, a C-terminal macrocycle of Formula (IIIb) forms the structure:

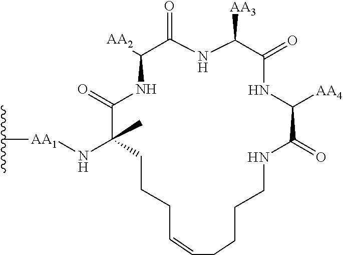

In some embodiments, a peptidomimetic macrocycle is provided comprising an amino acid sequence of formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH, or a C-terminal capping group; X₁-X₃₆ are absent or are amino acids, such that at least X₇-X₂₂ are not absent. In some embodiments, at least three, four, five, six, or seven amino acids from the group consisting of X₂₀, X₂₃, X₂₄, X₂₅, X₂₇, X₂₈, X₃₁, X₃₂, and X₃₄ are selected as follows: X₂₀ is Arg, X₂₃ is Trp or Phe, X₂₄ is Leu, X₂₅ is Arg, X₂₇ is Lys or Leu, X₂₈ is Leu or Ile, X₃₁ is Val or Ile, X₃₂ is His, and X₃₄ is Phe. In some embodiments, the peptidomimetic macrocycle comprises at least one pair of crosslinked amino acids selected from the group consisting of amino acids X₁-X₃₆.

In some embodiments, a peptidomimetic macrocycle is provided comprising an amino acid sequence of formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH, or a C-terminal capping group; X₁-X₃₆ are absent or are amino acids, such that at least X₇-X₂₂ are not absent. In some embodiments, at least three, four, five, six, or seven amino acids from the group consisting of X₂₀, X₂₃, X₂₄, X₂₅, X₂₇, X₂₈, X₃₁, X₃₂, and X₃₄ are selected as follows: X₂₀ is Arg or Cit or an analog thereof, X₂₃ is Trp or Phe or Ala or 1Nal or 2Nal, X₂₄ is Leu or Cpg or Cba or Ala or an analog thereof or a crosslinked amino acid, X₂₅ is Arg or His or Aib or Phe or Ser or Glu or Ala or Tyr or Trp or an analog thereof or a crosslinked amino acid, X₂₇ is Lys or Leu or Cit or Nle or hF or Tyr or His or Phe or Gln or an analog thereof or a crosslinked amino acid, X₂₈ is Leu or Ile or Cpg or Cba or Cha or an analog thereof or a crosslinked amino acid, X₃₁ is Val or Ile or Cpg or Cba or Nle or Thr or an analog thereof or a crosslinked amino acid, X₃₂ is His or Tyr or Phe or Ala or 2Pal or an analog thereof or a crosslinked amino acid, and X₃₄ is Phe or Tyr or Ala. In some embodiments, the peptidomimetic macrocycle comprises at least one pair of crosslinked amino acids selected from the group consisting of amino acids X₁-X₃₆.

In some embodiments, a peptidomimetic macrocycle is provided comprising an amino acid sequence of formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH or a C-terminal capping group; X₁-X₃₆ are absent or are amino acids, such that at least X₇-X₂₂ are not absent. In some embodiments, A is the amino acid sequence X₀-X₁-X₂-X₃-X₄-X₅-X₆-X₇-X₈-X₉-X₁₀-X₁₁-X₁₂-X₁₃-X₁₄ and comprises at least three amino acids selected from PTH (7-14). In some embodiments, B is the amino acid sequence X₁₅-X₁₆-X₁₇-X₁₈-X₁₉-X₂₀-X₂₁ and comprises at least three amino acids selected from PTHrP (15-21). In some embodiments, C is the amino acid sequence X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇ and comprises at least six amino acids selected from PTH (22-34). In some embodiments, the peptidomimetic macrocycle comprises at least one pair of crosslinked amino acids selected from the group consisting of amino acids X₁-X₃₆.

In some embodiments, a peptidomimetic macrocycle is provided comprising an amino acid sequence of formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH or a C-terminal capping group; X₁-X₃₆ are absent or are amino acids, such that at least X₇-X₂₂ are not absent. In some embodiments, A is the amino acid sequence X₀-X₁-X₂-X₃-X₄-X₅-X₆-X₇-X₈-X₉-X₁₀-X₁₁-X₁₂-X₁₃-X₁₄ and comprises at least two amino acids selected from PTHrP (7-14). In some embodiments, B is the amino acid sequence X₁₅-X₁₆-X₁₇-X₁₈-X₁₉-X₂₀-X₂₁ and comprises at least three amino acids selected from PTHrP (15-21). In some embodiments, C is the amino acid sequence X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇ and comprises at least three amino acids selected from PTH (22-34); and wherein the peptidomimetic macrocycle comprises at least one pair of crosslinked amino acids selected from the group consisting of amino acids X₁-X₃₆.

In some embodiments, a peptidomimetic macrocycle is provided comprising an amino acid sequence of formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH or a C-terminal capping group; X₁-X₃₆ are absent or are amino acids, such that at least X₇-X₂₂ are not absent; A is the amino acid sequence X₀-X₁-X₂-X₃-X₄-X₅-X₆-X₇-X₈-X₉-X₁₀-X₁₁-X₁₂-X₁₃-X₁₄ and comprises at least three amino acids selected from PTHrP (7-14) or at least three amino acids selected from PTHrP (7-14); wherein X₁₀ is not Asn or Asp; X₁₁ is not Asn or Asp, X₁₂ is not Gly, or any combination thereof; B is the amino acid sequence X₁₅-X₁₆-X₁₇-X₁₈-X₁₉-X₂₀-X₂₁ and comprises at least three amino acids selected from PTHrP (15-21); and C is the amino acid sequence X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇ and comprises at least three amino acids selected from PTHrP (22-36) or at least three amino acids selected from PTH (22-34); and wherein the peptidomimetic macrocycle comprises at least one pair of crosslinked amino acids selected from the group consisting of amino acids X₁-X₃₆.

In some embodiments, a peptidomimetic macrocycle is provided comprising an amino acid sequence of formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH or a C-terminal capping group; X₁—X₃₆ are absent or are amino acids, such that at least X₇-X₂₂ are not absent; A is the amino acid sequence X₀-X₁-X₂-X₃-X₄-X₅-X₆-X₇-X₈-X₉-X₁₀-X₁₁-X₁₂-X₁₃-X₁₄ and comprises at least two contiguous amino acids selected from PTHrP (7-14); B is the amino acid sequence X₁₅-X₁₆-X₁₇-X₁₈-X₁₉-X₂₀-X₂₁ and comprises at least three contiguous amino acids selected from PTHrP (15-21); and C is the amino acid sequence X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇ and comprises at least two contiguous amino acids selected from PTHrP (22-36) or at least two contiguous amino acids selected from PTH (22-34); and wherein the peptidomimetic macrocycle comprises at least one pair of crosslinked amino acids selected from the group consisting of amino acids X₁-X₃₆.

In some embodiments, X₀ is —H or an N-terminal capping group. In some embodiments, X₁-X₆ are absent or are amino acids. In some embodiments, X₃₇ is —OH, or a C-terminal capping group. In some embodiments, X₃₅-X₃₆ are absent or are amino acids. In some embodiments, the peptidomimetic macrocycle comprises at least one macrocycle-forming linker connecting a pair of amino acids selected from the group consisting of amino acids X₇-X₃₄. In some embodiments, X₁₃ and X₁₇ are crosslinked. In some embodiments, X₉ and X₁₃ are crosslinked. In some embodiments, X₁₈ and X₂₂ are crosslinked. In some embodiments, X₂₄ and X₂₈ are crosslinked.

In some embodiments, X₀ is —H or an N-terminal capping group, for example acetyl, 1NaAc, 2NaAc, PhAc, a fatty acid, a urea, a sulfonamide, or a polyalkylene oxide linked to the N-terminus of residue X₁.

In some embodiments, X₁ is Ser, Ala, Deg, Har, a dialkylated amino acid, Aib, Ac5c, Ac3c, Ac6c, desamino-Ser, desamino-Ac5c, desamino-Aib, Val, an analog thereof, or absent. In some embodiments, X₂ is an aromatic amino acid, Val, Trp, Arg, D-Trp, D-Arg, F4COOH, Bip, F4NH₂, 1Nal, 2Nal, 2Pal, 3Pal, 4Pal, Bpa, Deg, Ile, an analog thereof, or absent. In some embodiments, X₃ is Ser, Deg, Aib, Ac3c, Ac5c, Ac6c, Glu, Lys, Phe, Aib, Gly, Ala, an analog thereof, or absent. In some embodiments, X₄ is Glu, Gln, Phe, His, an analog thereof, or absent. In some embodiments, X₅ is Ile, His, Lys, Glu, Phe, an analog thereof, or absent. In some embodiments, X₆ is Gln, Lys, Glu, Phe, Ala, an analog thereof, or absent. In some embodiments, X₇ is an aromatic amino acid, a hydrophobic amino acid, Leu, Lys, Glu, Ala, Phe, Met, F4Cl, 1NaI, 2Nal, 2Pal, 3Pal, 4Pal, Phe, Nle, an analog thereof, or a crosslinked amino acid. In some embodiments, X₈ is a hydrophobic amino acid, Met, Leu, Nle, an analog thereof, or a crosslinked amino acid. In some embodiments, X₉ is an aromatic amino acid, His, Aib, or an analog thereof. In some embodiments, X₁₀ is Asn, Asp, Gln, Ala, Ser, Val, His, Trp, Aib, an analog thereof, or a crosslinked amino acid. In some embodiments, X₁₁ is a hydrophobic amino acid, a positively charged amino acid, an aromatic amino acid, Leu, Lys, Har, Arg, Ala, Val, Ile, Met, Phe, Trp, D-Trp, Nle, Cit, hK, hL, an analog thereof, or a crosslinked amino acid. In some embodiments, X₁₂ is a D-amino acid, a hydrophobic amino acid, a hydrophilic amino acid, an aromatic amino acid, a positively charged amino acid, a negatively charged amino acid, an uncharged amino acid, Gly, D-Trp, Ala, Aib, Arg, His, Trp, an analog thereof, or a crosslinked amino acid. In some embodiments, X₁₃ is a positively charged amino acid, Lys, Ser, Ala, Aib, Leu, Glu, Gln, Arg, His, Phe, Trp, Pro, Cit, Kfam, Ktam, an analog thereof, or a crosslinked amino acid. In some embodiments, X₁₄ is an aromatic amino acid, His, Ser, Trp, Ala, Leu, Lys, Arg, Phe, Trp, Aib, an analog thereof, or a crosslinked amino acid. In some embodiments, X₁₅ is a hydrophobic amino acid, Leu, Ile, Tyr, Aib, an analog thereof, or a crosslinked amino acid. In some embodiments, X₁₆ is Asn, Gln, Lys, Ala, Glu, an analog thereof, or a crosslinked amino acid. In some embodiments, X₁₇ is Ser, Asp, β-Ala, β-hPhe, Aib, an analog thereof, or a crosslinked amino acid. In some embodiments, X₁₈ is a hydrophobic amino acid, Met, Nle, Leu, β-hIle, hSer(OMe), β-hPhe, Aib, an analog thereof, or a crosslinked amino acid. In some embodiments, X₁₉ is a positively charged amino acid, Glu, Arg, Ser, Aib, Cit, Glu, Ala, an analog thereof, or a crosslinked amino acid. In some embodiments, X₂₀ is a positively charged amino acid, Cit, Arg, Ala, an analog thereof, or a crosslinked amino acid. In some embodiments, X₂₁ is a positively charged amino acid, Cit, Val, Arg, Lys, Gln, Cit, Ala, an analog thereof, or a crosslinked amino acid. In some embodiments, X₂₂ is an aromatic amino acid, Glu, Phe, Ser, Aib, an analog thereof, or a crosslinked amino acid. In some embodiments, X₂₃ is an aromatic amino acid, a hydrophobic amino acid, Trp, Phe, Ala, 9-Aal, 1Nal, 2Nal, an analog thereof, absent, or a crosslinked amino acid. In some embodiments, X₂₄ is an aromatic amino acid, a hydrophobic amino acid, Leu, Ala, Cba, Cpg, Aib, an analog thereof, absent, or a crosslinked amino acid. In some embodiments, X₂₅ is a positively charged amino acid, Cit, Arg, His, Leu, Trp, Tyr, Phe, Ala, Ser, Glu, Aib, an analog thereof, absent, or a crosslinked amino acid. In some embodiments, X₂₆ is a positively charged amino acid, Lys, His, Ala, Phe, Ser, Glu, AmO, AmK, Cit, and Aib an analog thereof, absent, or a crosslinked amino acid. In some embodiments, X₂₇ is a positively charged amino acid, Cit, Lys, Leu, Arg, Nle, Tyr, His, Phe, hF, Leu, Gln, an analog thereof, absent, or a crosslinked amino acid. In some embodiments, X₂₈ is an aromatic amino acid, a hydrophobic amino acid, Leu, Ile, Cba, Cha, Cpg, Aib, an analog thereof, absent, or a crosslinked amino acid. In some embodiments, X₂₉ is Gln, Ala, Glu, Ser, Aib, an analog thereof, absent, or a crosslinked amino acid. In some embodiments, X₃₀ is Asp, Glu, Leu, Arg, hPhe, Asn, His, Ser, Ala, Phe, an analog thereof, absent, or a crosslinked amino acid. In some embodiments, X₃₁ is an aromatic amino acid, a hydrophobic amino acid, Val, Ile, Nle, Thr, Ser, Cba, Cpg, an analog thereof, absent, or a crosslinked amino acid. In some embodiments, X₃₂ is an aromatic amino acid, His, Trp, Arg, Phe, Tyr, Ile, Ala, 2Pal, 3Pal, 4Pal, an analog thereof, absent, or a crosslinked amino acid. In some embodiments, X₃₃ is Asn, Thr, Glu, Asp, Lys, Phe, an analog thereof, absent, or a crosslinked amino acid. In some embodiments, X₃₄ is an aromatic amino acid, a hydrophobic amino acid, Phe, Ala, Tyr, Arg, 2Nal, hF, Glu, Lys, Ser, an analog thereof, absent, or a crosslinked amino acid. In some embodiments, X₃₅ is Glu, Gly, an analog thereof, absent, or a crosslinked amino acid. In some embodiments, X₃₆ is an aromatic amino acid, Tyr, Pra, an analog thereof, absent, or a crosslinked amino acid. In some embodiments, X₃₇ is —OH, or a C-terminal capping group, for example a primary, secondary, or tertiary amino group, an alkyloxy or an aryloxy group.

In some embodiments, X₁₉ is Cit or Arg, X₂₀ is Cit or Arg, and X₂₁ is Cit or Arg.

In some embodiments, X₉ and X₁₃ are crosslinked amino acids. In some embodiments, X₁₀ and X₁₄ are crosslinked amino acids. In some embodiments, X₁₁ and X₁₅ are crosslinked amino acids. In some embodiments, X₁₂ and X₁₆ are crosslinked amino acids. In some embodiments, X₁₃ and X₁₇ are crosslinked amino acids. In some embodiments, X₁₄ and X₁₈ are crosslinked amino acids. In some embodiments, X₁₈ and X₂₂ are crosslinked amino acids. In some embodiments, X₂₂ and X₂₆ are crosslinked amino acids. In some embodiments, X₂₄ and X₂₈ are crosslinked amino acids. In some embodiments, X₂₆ and X₃₀ are crosslinked amino acids. In some embodiments, X₂₇ and X₃₁ are crosslinked amino acids.

In some embodiments, the peptidomimetic macrocycle comprises two pairs of crosslinked amino acids. In some embodiments, X₁₃ and X₁₇ are crosslinked amino acids, and X₂₆ and X₃₀ are crosslinked amino acids. In some embodiments, X₁₄ and X₁₈ are crosslinked amino acids, and X₂₆ and X₃₀ are crosslinked amino acids. In some embodiments, X₁₄ and X₁₈ are crosslinked amino acids, and X₂₂ and X₂₆ are crosslinked amino acids. In some embodiments, X₁₄ and X₁₈ are crosslinked amino acids, and X₂₄ and X₂₈ are crosslinked amino acids. In some embodiments, X₁₄ and X₁₈ are crosslinked amino acids, and X₂₇ and X₃₁ are crosslinked amino acids.

In some embodiments, X₁-X₆ are absent. In some embodiments, X₃₅-X₃₆ are absent.

In some embodiments, X₁₁ is Har. In some embodiments, X₁₁ is Leu. In Some embodiments, X₁₉ is a positively charged amino acid, Cit, Arg. or an analog thereof. In some embodiments, X₁₉ is Arg. In some embodiments, X₂₀ is a positively charged amino acid, Cit, Arg, or an analog thereof. In some embodiments, X₂₀ is Arg. In some embodiments, X₂₁ is a positively charged amino acid, Cit, Arg, or an analog thereof. In some embodiments, X₂₁ is Arg. In some embodiments, X₂₃ is Trp. In some embodiments, X₂₃ is Phe. In some embodiments, X₂₄ is Leu. In some embodiments, X₂₅ is Arg. In some embodiments, X₂₇ is Lys. In some embodiments, X₂₇ is Leu. In some embodiments, X₂₈ is Leu. In some embodiments, X₂₈ is Ile. In some embodiments, X₃₁ is Val. In some embodiments, X₃₁ is Ile. In some embodiments, X₃₂ is His. In some embodiments, X₃₄ is Phe.

In some embodiments, X₂₀ is Arg, X₂₃ is Trp, X₂₄ is Leu, X₂₅ is Arg, X₂₇ is Lys, X₂₈ is Leu, X₃₁ is Val, and X₃₄ is Phe. In some embodiments, X₂₀ is Arg, X₂₃ is Phe, X₂₄ is Leu, X₂₇ is Leu, X₂₈ is Ile, X₃₁ is Ile, and X₃₂ is His.

In some embodiments, a peptidomimetic macrocycle is provided comprising an amino acid sequence of formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein A is an amino acid sequence comprising at least three amino acids selected from PTH (7-14);
   B is an amino acid sequence comprising at least three amino acids selected from PTHrP (15-21); and C is an amino acid sequence comprising at least six amino acids selected from PTH (22-34); wherein the peptidomimetic macrocycle comprises at least one macrocycle-forming linker.

In some embodiments, A is X₀-X₁-X₂-X₃-X₄-X₅-X₆-X₇-X₈-X₉-X₁₀-X₁₁-X₁₂-X₁₃-X₁₄. In some embodiments, A is X₀-X₇-X₈-X₉-X₁₀-X₁₁-X₁₂-X₁₃-X₁₄. In some embodiments, B is X₁₅-X₁₆-X₁₇-X₁₈-X₁₉-X₂₀-X₂₁. In some embodiments, C is X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇. In some embodiments, C is X₂₂-X₂₃-X₂₄-X₂₅-X₂₆-X₂₇-X₂₈-X₂₉-X₃₀-X₃₁-X₃₂-X₃₃-X₃₄-X₃₇.

In some embodiments, the peptidomimetic macrocycle comprises a helix. In some embodiments, the peptidomimetic macrocycle comprises an α-helix. In some embodiments, the peptidomimetic macrocycle comprises an α,α-disubstituted amino acid. In some embodiments, each amino acid connected by the macrocycle-forming linker is an α,α-disubstituted amino acid.

In some embodiments, the peptidomimetic macrocycle comprises at least one α-helix motif. For example, A, B and/or C in the compound can include one or more α-helices. As a general matter, α-helices include between 3 and 4 amino acid residues per turn. In some embodiments, the α-helix of the peptidomimetic macrocycle includes 1-5 turns and, therefore, 3-20 amino acid residues. In specific embodiments, the α-helix includes 1 turn, 2 turns, 3 turns, 4 turns, or 5 turns. In some embodiments, the macrocycle-forming linker stabilizes an α-helix motif included within the peptidomimetic macrocycle. Thus, in some embodiments, the length of the macrocycle-forming linker L from a first Cα to a second Cα is selected to increase the stability of an α-helix. In some embodiments, the macrocycle-forming linker spans from 1-5 turns of the α-helix. In some embodiments, the macrocycle-forming linker spans approximately 1 turn, 2 turns, 3 turns, 4 turns, or 5 turns of the α-helix. In some embodiments, the length of the macrocycle-forming linker is approximately 5-9 Å per turn of the α-helix, or approximately 6-8 Å per turn of the α-helix. Where the macrocycle-forming linker spans approximately 1 turn of an α-helix, the length is equal to approximately 5-13 carbon-carbon bonds, approximately 7-11 carbon-carbon bonds, or approximately 9 carbon-carbon bonds. Where the macrocycle-forming linker spans approximately 2 turns of an α-helix, the length is equal to approximately 8-16 carbon-carbon bonds, approximately 10-14 carbon-carbon bonds, or approximately 12 carbon-carbon bonds. Where the macrocycle-forming linker spans approximately 3 turns of an α-helix, the length is equal to approximately 14-22 carbon-carbon bonds, approximately 16-20 carbon-carbon bonds, or approximately 18 carbon-carbon bonds. Where the macrocycle-forming linker spans approximately 4 turns of an α-helix, the length is equal to approximately 20-28 carbon-carbon bonds, approximately 22-26 carbon-carbon bonds, or approximately 24 carbon-carbon bonds. Where the macrocycle-forming linker spans approximately 5 turns of an α-helix, the length is equal to approximately 26-34 carbon-carbon bonds, approximately 28-32 carbon-carbon bonds, or approximately 30 carbon-carbon bonds. Where the macrocycle-forming linker spans approximately 1 turn of an α-helix, the linkage contains approximately 4-12 atoms, approximately 6-10 atoms, or approximately 8 atoms. Where the macrocycle-forming linker spans approximately 2 turns of the α-helix, the linkage contains approximately 7-15 atoms, approximately 9-13 atoms, or approximately 11 atoms. Where the macrocycle-forming linker spans approximately 3 turns of the α-helix, the linkage contains approximately β-21 atoms, approximately 15-19 atoms, or approximately 17 atoms. Where the macrocycle-forming linker spans approximately 4 turns of the α-helix, the linkage contains approximately 19-27 atoms, approximately 21-25 atoms, or approximately 23 atoms. Where the macrocycle-forming linker spans approximately 5 turns of the α-helix, the linkage contains approximately 25-33 atoms, approximately 27-31 atoms, or approximately 29 atoms. Where the macrocycle-forming linker spans approximately 1 turn of the α-helix, the resulting macrocycle forms a ring containing approximately 17-25 members, approximately 19-23 members, or approximately 21 members. Where the macrocycle-forming linker spans approximately 2 turns of the α-helix, the resulting macrocycle forms a ring containing approximately 29-37 members, approximately 31-35 members, or approximately 33 members. Where the macrocycle-forming linker spans approximately 3 turns of the α-helix, the resulting macrocycle forms a ring containing approximately 44-52 members, approximately 46-50 members, or approximately 48 members. Where the macrocycle-forming linker spans approximately 4 turns of the α-helix, the resulting macrocycle forms a ring containing approximately 59-67 members, approximately 61-65 members, or approximately 63 members. Where the macrocycle-forming linker spans approximately 5 turns of the α-helix, the resulting macrocycle forms a ring containing approximately 74-82 members, approximately 76-80 members, or approximately 78 members.

In other embodiments, the length of the macrocycle-forming linker -L₁-L₂-as measured from a first Cα to a second Cα is selected to stabilize a desired secondary peptide structure, such as an α-helix formed by residues of the peptidomimetic macrocycle including, but not necessarily limited to, those between the first Cα to a second Cα.

In some embodiments, a peptidomimetic macrocycle comprises a macrocycle-forming linker connecting a backbone amino group of a first amino acid to a second amino acid within the peptidomimetic macrocycle.

Exemplary macrocycle-forming linkers -L₁-L₂-are shown below.

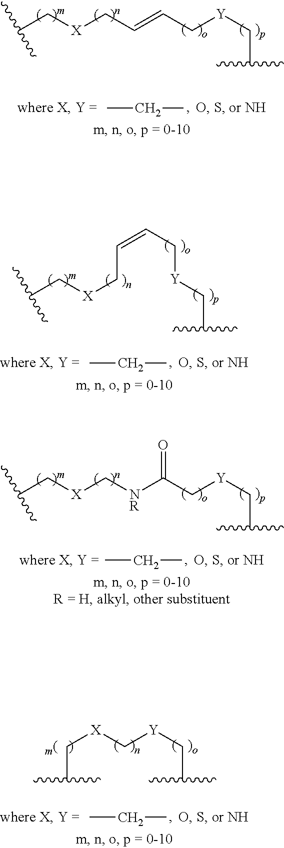

In some embodiments, L is a macrocycle-forming linker of the formula

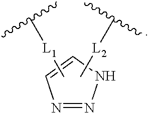

Exemplary embodiments of such macrocycle-forming linkers L are shown below.

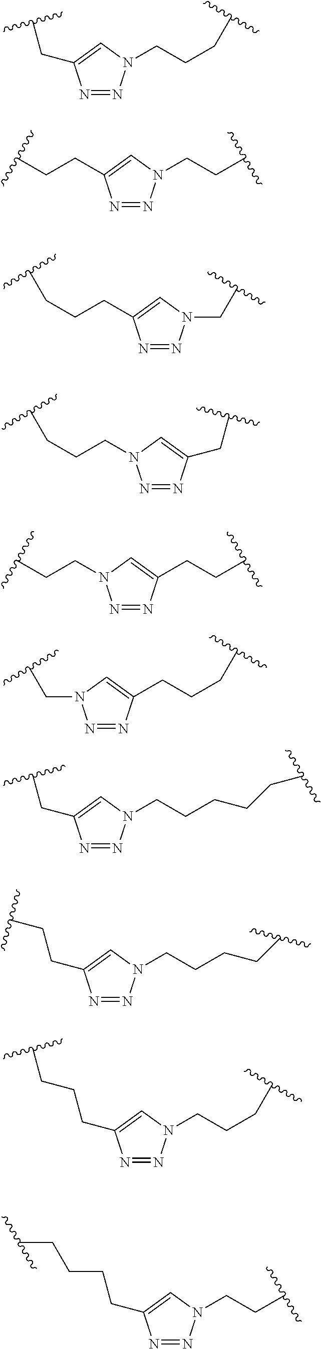

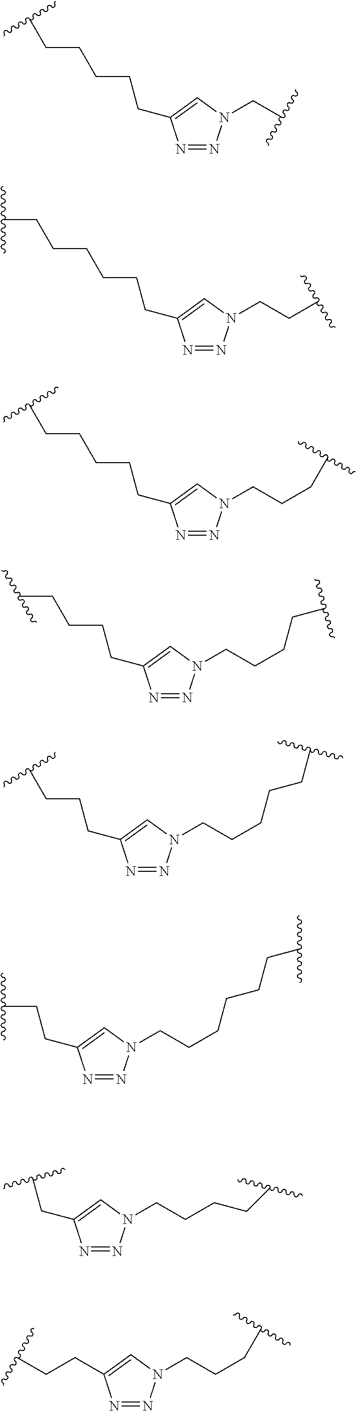

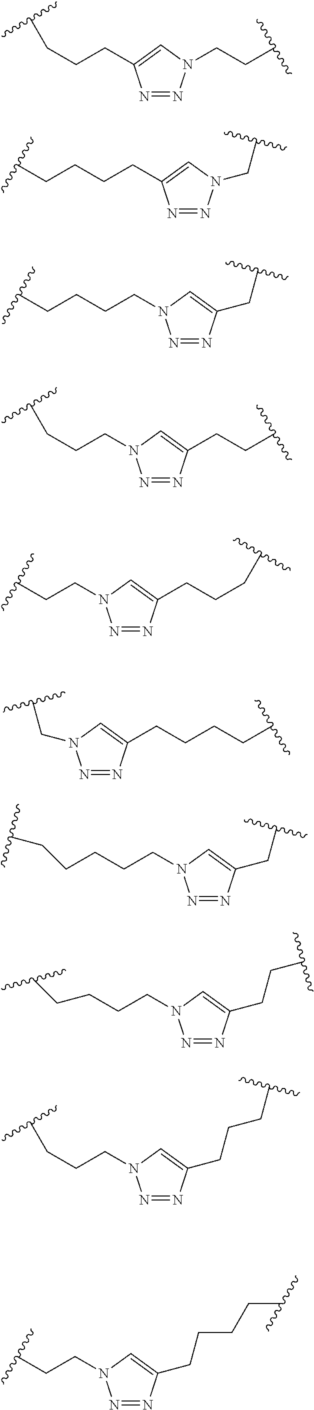

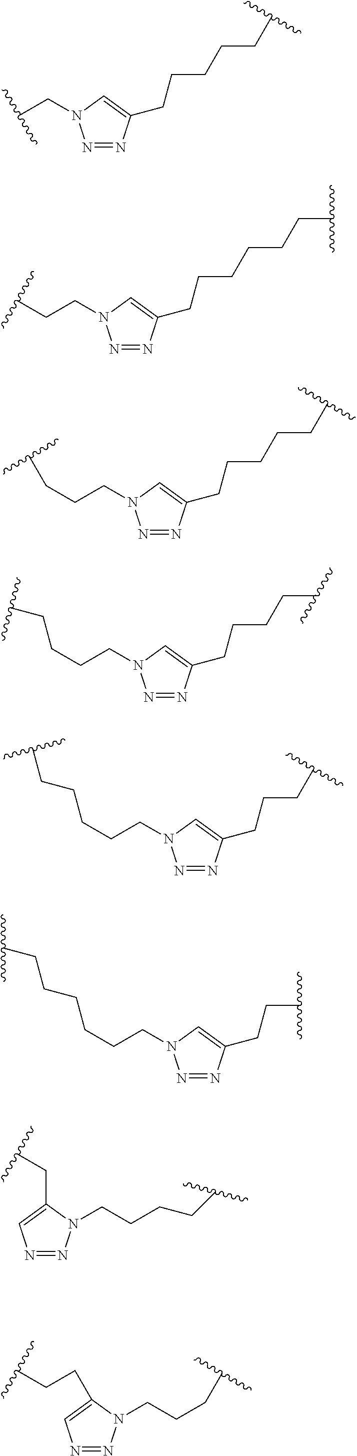

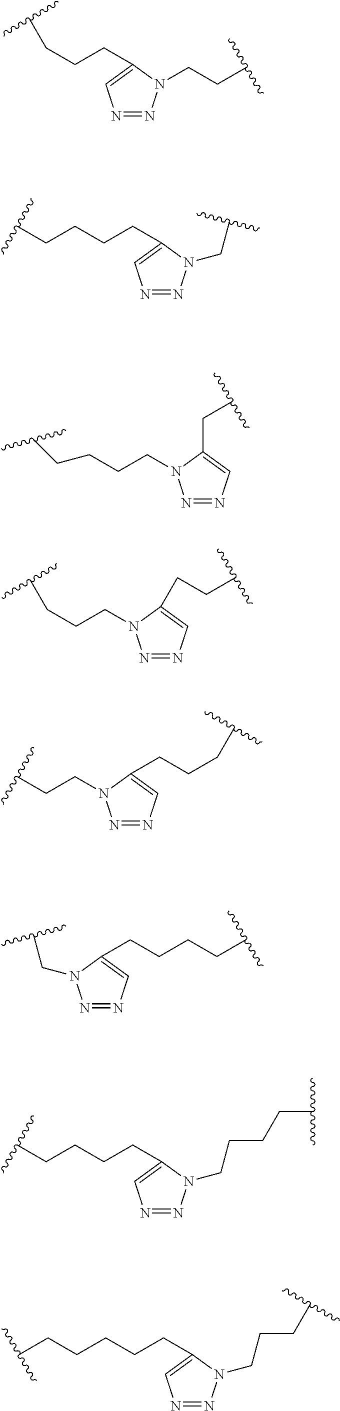

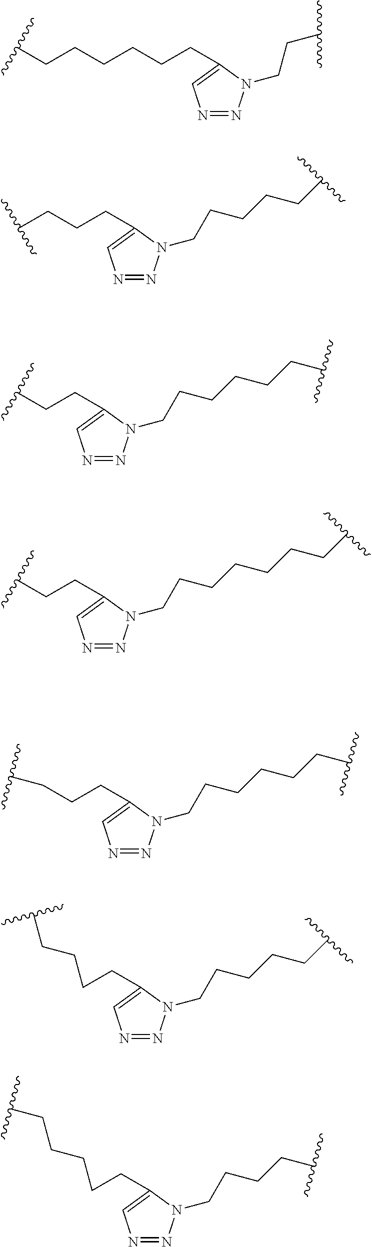

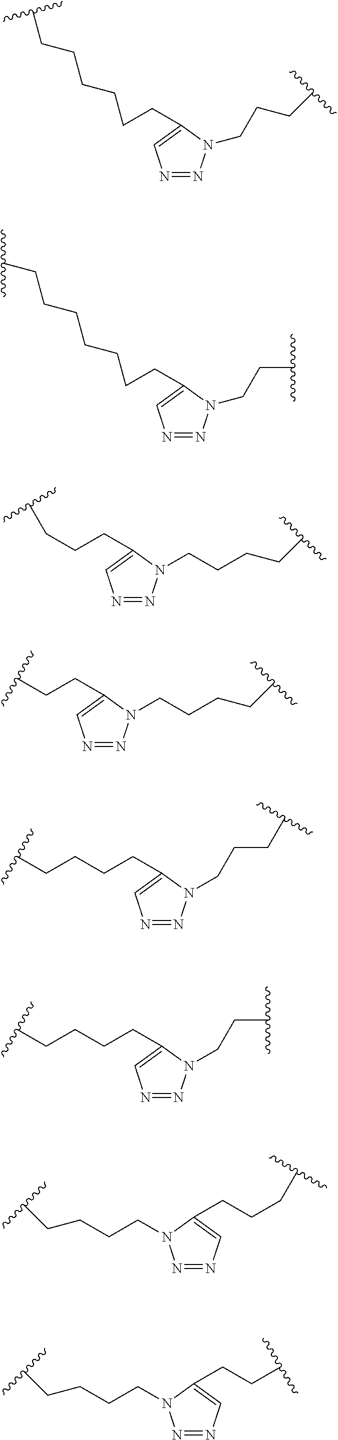

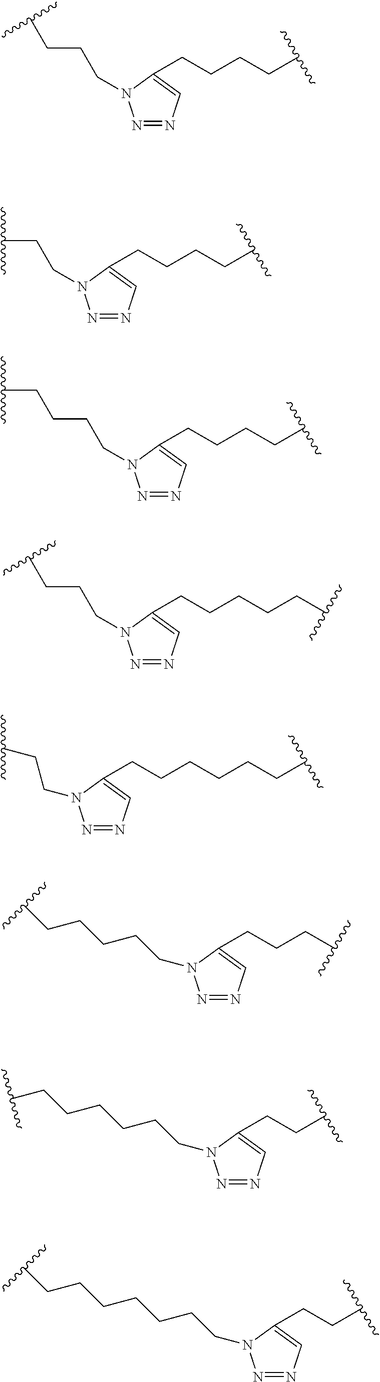

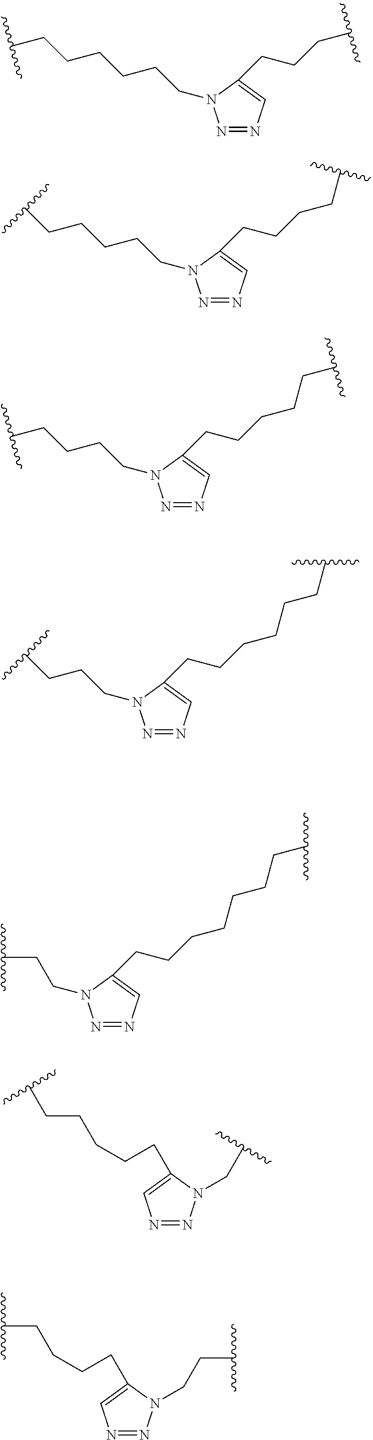

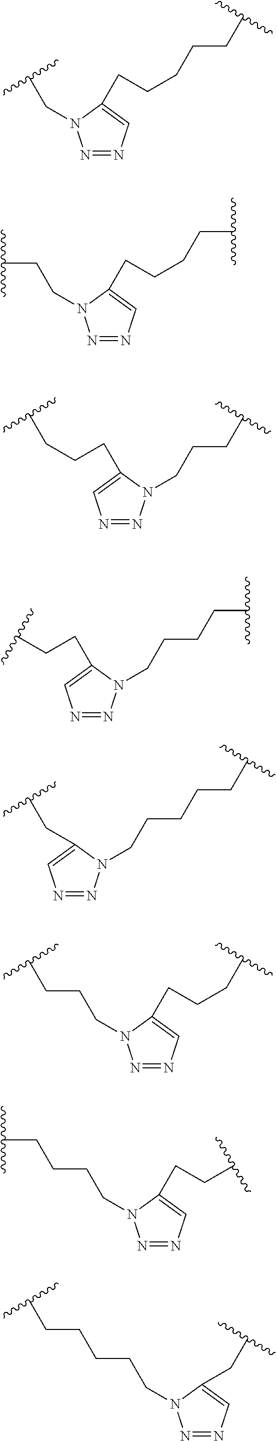

Pharmaceutical formulations are provided comprising an effective amount of a peptidomimetic macrocycle described herein. The peptidomimetic macrocycles provided herein are cross-linked (e.g., stapled) and possess improved pharmaceutical properties relative to their corresponding uncross-linked peptidomimetic macrocycles. These improved properties include improved bioavailability, enhanced chemical and in vivo stability, increased potency, and reduced immunogenicity (i.e. fewer or less severe injection site reactions). Also provided herein is a composition comprising a peptidomimetic macrocycle comprising an amino acid sequence that has at least about 60%, 65%, 70%, 75%, 80%, 85%, 90%, 95%, 96%, 97%, 98%, 99% or 100% sequence identity to a sequence of Table 1a, wherein the peptidomimetic macrocycle comprises a macrocycle-forming linker, wherein the macrocycle-forming linker connects amino acids 24 and 28 or 27 and 31. Also provided herein is a composition comprising a peptidomimetic macrocycle comprising an amino acid sequence that has at least about 60%, 65%, 70%, 75%, 80%, 85%, 90%, 95%, 96%, 97%, 98%, 99% or 100% sequence identity to a sequence of Table 6, wherein the peptidomimetic macrocycle comprises at least two amino acids connected by a macrocycle-forming linker.

Also provided herein is a composition comprising a peptidomimetic macrocycle comprising an amino acid sequence that has at least about 60%, 65%, 70%, 75%, 80%, 85%, 90%, 95%, 96%, 97%, 98%, 99% or 100% sequence identity to a sequence of Table 1a, 1b, 2a, or 2b, wherein the peptidomimetic macrocycle comprises at least two non-natural amino acids connected by a macrocycle-forming linker. Also provided herein is a composition comprising a peptidomimetic macrocycle comprising an amino acid sequence that has at least about 60%, 65%, 70%, 75%, 80%, 85%, 90%, 95%, 96%, 97%, 98%, 99% or 100% sequence identity to a sequence of Table 1a, wherein the peptidomimetic macrocycle comprises at least one macrocycle-forming linker, wherein the macrocycle-forming linker connects amino acids 10 and 14 or 11 and 15.

In some embodiments, the at least one macrocycle-forming linker connects amino acids 7 and 11, 7 and 14, 8 and 12, 9 and 13, 10 and 14, 11 and 15, 12 and 16, 13 and 17, 14 and 18, 14 and 21, 15 and 19, 15 and 22, 17 and 24, 18 and 22, 18 and 25, 22 and 26, 22 and 29, 24 and 28, 25 and 32, 26 and 30, 26 and 33, or 27 and 31. In some embodiments, the at least one macrocycle-forming linker connects amino acids 7 and 11, 8 and 12, 9 and 13, 10 and 14, 13 and 17, 14 and 18, or 18 and 22. In some embodiments, the at least one macrocycle-forming linker connects amino acids 9 and 13. In some embodiments, the macrocycle-forming linker connects amino acids 10 and 14 or 11 and 15. In some embodiments, the at least one macrocycle-forming linker connects amino acids 13 and 17. In some embodiments, the at least one macrocycle-forming linker connects amino acids 14 and 18. In some embodiments, the at least one macrocycle-forming linker connects amino acids 18 and 22. In some embodiments, the macrocycle-forming linker connects amino acids 24 and 28 or 27 and 31. In some embodiments, the peptidomimetic macrocycle comprises a second macrocycle-forming linker. In some embodiments, the second macrocycle-forming linker connects amino acids 18 and 22, 22 and 26, 24 and 28, or 26 and 30. In some embodiments, the second macrocycle-forming linker connects amino acids 22 and 26. In some embodiments, the second macrocycle-forming linker connects amino acids 24 and 28. In some embodiments, the second macrocycle-forming linker connects amino acids 26 and 30. In some embodiments, the first macrocycle-forming linker connects amino acids 7 and 11, and the second macrocycle-forming linker connects amino acids 22 and 26. In some embodiments, the first macrocycle-forming linker connects amino acids 8 and 12, and the second macrocycle-forming linker connects amino acids 22 and 26. In some embodiments, the peptidomimetic macrocycle comprises a second macrocycle-forming linker connecting amino acids 18 and 22 or 24 and 28. In some embodiments, the peptidomimetic macrocycle comprises a third macrocycle-forming linker. In some embodiments, the third macrocycle-forming linker connects amino acids 27-31. In some embodiments, the first macrocycle-forming linker connects amino acids 13 and 17, and the second macrocycle-forming linker connects amino acids 22 and 26. In some embodiments, the first macrocycle-forming linker connects amino acids 13 and 17, and the second macrocycle-forming linker connects amino acids 24 and 28. In some embodiments, the first macrocycle-forming linker connects amino acids 14 and 18, and the second macrocycle-forming linker connects amino acids 22 and 26.

In some embodiments, the at least one macrocycle-forming linker is a straight chain alkenyl. In some embodiments, the at least one macrocycle-forming linker is a straight chain alkenyl with 6 to 14 carbon atoms. In some embodiments, the at least one macrocycle-forming linker is a straight chain alkenyl with 8 to 12 carbon atoms, for example 8, 9, 10, 11 or 12 carbon atoms. In some embodiments, the at least one macrocycle-forming linker is a C₈ alkenyl with a double bond between C₄ and C₅ of the C₈ alkenyl. In some embodiments, the at least one macrocycle-forming linker is a C₁₂ alkenyl with a double bond between C₄ and C₅ or C₅ and C₆ of the C₁₂ alkenyl.

In some embodiments, the at least one macrocycle-forming linker comprises a first and a second macrocycle-forming linker, wherein the first macrocycle-forming linker connects a first and a second amino acid, wherein the second macrocycle-forming linker connects a third and a fourth amino acid, wherein the first amino acid is upstream of the second amino acid, the second amino acid is upstream of the third amino acid, and the third amino acid is upstream of the fourth amino acid. In some embodiments, 1, 2, 3, 4, 5, 6, or 7, amino acids are between the second and third amino acids. In some embodiments, 4 or 5 amino acids are between the second and third amino acids.

In some embodiments, the at least one macrocycle-forming linker comprises a first and a second macrocycle-forming linker that are separated by 2, 3, 4, 5, 6, or 7 amino acids. In some embodiments, the at least one macrocycle-forming linker comprises a first and a second macrocycle-forming linker that are separated by 4 or 5 amino acids.

In some embodiments, the peptidomimetic macrocycle contains 16-36 amino acids, for example 16, 17, 18, 19, 20, 21, 22, 23, 24, 25, 26, 27, 28, 29, 30, 31, 32, 33, 34, 35 or 36 amino acids. In some embodiments, the peptidomimetic macrocycle contains 24-36 amino acids, for example 24, 25, 26, 27, 28, 29, 30, 31, 32, 33, 34, 35 or 36 amino acids.

In some embodiments, the peptidomimetic macrocycle has an amino acid sequence with at least about 60%, 65%, 70%, 75%, 80%, 85%, 90%, 95%, 96%, 97%, 98%, 99% or 100% sequence identity to a sequence of Table 7. In some embodiments, the peptidomimetic macrocycle has an amino acid sequence with at least about 75% sequence identity to a sequence of Table 7. In some embodiments, the peptidomimetic macrocycle has an amino acid sequence with at least about 90% sequence identity to a sequence of Table 7. In some embodiments, the peptidomimetic macrocycle has an amino acid sequence with at least about 95% sequence identity to a sequence of Table 7. In some embodiments, the peptidomimetic macrocycle has an amino acid sequence with 100% sequence identity to a sequence of Table 7.

In some embodiments, the peptidomimetic macrocycle has an amino acid sequence with at least about 60%, 65%, 70%, 75%, 80%, 85%, 90%, 95%, 96%, 97%, 98%, 99% or 100% sequence identity to a sequence of Table 3b. In some embodiments, the peptidomimetic macrocycle has an amino acid sequence with at least about 75% sequence identity to a sequence of Table 3b. In some embodiments, the peptidomimetic macrocycle has an amino acid sequence with at least about 90% sequence identity to a sequence of Table 3b. In some embodiments, the peptidomimetic macrocycle has an amino acid sequence with at least about 95% sequence identity to a sequence of Table 3b. In some embodiments, the peptidomimetic macrocycle has an amino acid sequence with 100% sequence identity to a sequence of Table 3b.

In some embodiments, the peptidomimetic macrocycle has an amino acid sequence with at least about 60%, 65%, 70%, 75%, 80%, 85%, 90%, 95%, 96%, 97%, 98%, 99% or 100% sequence identity to a sequence of Table 6. In some embodiments, the peptidomimetic macrocycle has an amino acid sequence with at least about 75% sequence identity to a sequence of Table 6. In some embodiments, the peptidomimetic macrocycle has an amino acid sequence with at least about 90% sequence identity to a sequence of Table 6. In some embodiments, the peptidomimetic macrocycle has an amino acid sequence with at least about 95% sequence identity to a sequence of Table 6. In some embodiments, the peptidomimetic macrocycle has an amino acid sequence with 100% sequence identity to a sequence of Table 6.

In some embodiments, the peptidomimetic macrocycle has an amino acid sequence with at least about 75% sequence identity to a sequence of Table 8. In some embodiments, the peptidomimetic macrocycle has an amino acid sequence with at least about 90% sequence identity to a sequence of Table 8. In some embodiments, the peptidomimetic macrocycle has an amino acid sequence with at least about 95% sequence identity to a sequence of Table 8. In some embodiments, the peptidomimetic macrocycle has an amino acid sequence with 100% sequence identity to a sequence of Table 8. In some embodiments, the peptidomimetic macrocycle has a structure of a peptidomimetic macrocycle of Table 8.

In some embodiments, the peptidomimetic macrocycle has the Formula:

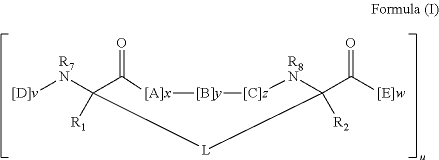

wherein: each A, C, D, and E is independently an amino acid;
each B is independently an amino acid,

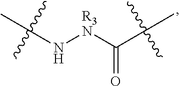

[—NH-L₃-CO—], [—NH-L₃-SO₂—], or [—NH-L₃-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or at least one of R₁ and R₂ forms a macrocycle-forming linker L′ connected to the alpha position of one of said D or E amino acids;
each R₃ is independently —H, alkyl, alkenyl, alkynyl, arylalkyl, heteroalkyl, cycloalkyl, heterocycloalkyl, cycloalkylalkyl, aryl, or heteroaryl, optionally substituted with R₅;
each L or L′ is independently a macrocycle-forming linker of the formula -L₁-L₂-,

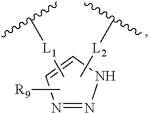

or -L₁-S-L₂-S-L₃-;
each L₁, L₂ and L₃ is independently alkylene, alkenylene, alkynylene, heteroalkylene, cycloalkylene, heterocycloalkylene, arylene, heteroarylene, or [—R₄—K—R₄—]ₙ, each being optionally substituted with R₅; when L is not

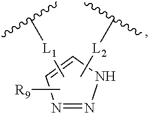

or -L₁-S-L₂-S-L₃-,
each L₁ and L₂ is independently alkylene, alkenylene, alkynylene, heteroalkylene, cycloalkylene, heterocycloalkylene, arylene, or heteroarylene;
each R₄ is independently alkylene, alkenylene, alkynylene, heteroalkylene, cycloalkylene, heterocycloalkylene, arylene, or heteroarylene;
each K is O, S, SO, SO₂, CO, CO₂ or CONR₃;
   each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
   each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
   each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue; R₉ is alkyl, alkenyl, alkynyl, aryl, cycloalkyl, cycloalkenyl, heteroaryl, or heterocyclyl group, unsubstituted or optionally substituted with Rₐ and/or R_{b}; Rₐ and R_{b} are independently alkyl, OCH₃, CF₃, NH₂, CH₂NH₂, F, Br, I,

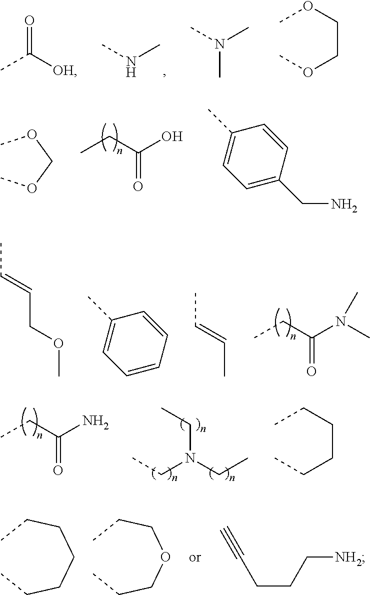

each v and w is independently an integer from 0-1000, for example 0-500, 0-200, 0-100, 0-50, 0-30, 0-20, or 0-10;
u is an integer from 1-10, for example 1-5, 1-3 or 1-2;
each x, y and z is independently an integer from 0-10, for example the sum of x+y+z is 2, 3, 6 or 10; n is an integer from 1-5; and
wherein A, B, C, D, and E, taken together with the crosslinked amino acids connected by the macrocycle-forming linker -L₁-L₂-, form an amino acid sequence of the peptidomimetic macrocycle with at least about 60%, 65%, 70%, 75%, 80%, 85%, 90%, 95%, 96%, 97%, 98%, 99%, or 100% sequence identity to a sequence of Table 1a, 1b, 2a, or 2b. In some embodiments, the peptidomimetic macrocycle comprises an amino acid sequence with at least about 60%, 65%, 70%, 75%, 80%, 85%, 90%, 95%, 96%, 97%, 98%, 99%, or 100% sequence identity to a sequence of Table 1a or 3a.

In some embodiments, the peptidomimetic macrocycle comprises an amino acid sequence with at least about 60%, 65%, 70%, 75%, 80%, 85%, 90%, 95%, 96%, 97%, 98%, 99%, or 100% sequence identity to a sequence of Table 6 or Table 7. In some embodiments, u is 1. In some embodiments, the sum of x+y+z is 2, 3 or 6. In some embodiments, the sum of x+y+z is 3 or 6. In some embodiments, each of v and w is independently an integer from 0 to 10, 0 to 15, 0 to 20, 0 to 25, or 0-30. In some embodiments, each of v and w is independently an integer from 0 to 20. In some embodiments, L₁ and L₂ are independently alkylene, alkenylene or alkynylene. In some embodiments, L₁ and L₂ are independently C₃-C₁₀ alkylene or alkenylene. In some embodiments, L₁ and L₂ are independently C₃-C₆ alkylene or alkenylene. In some embodiments, L is

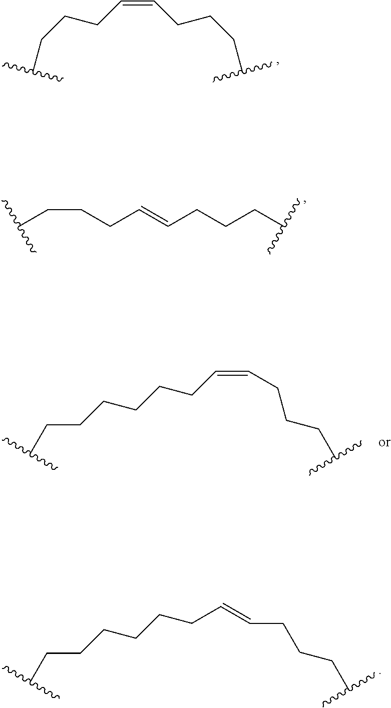 [SMILES: *CCC/C=C\CCC* ; *CCC/C=C/CCC* ; *CCC/C=C\CCCCCC* ; *CCC/C=C/CCCCCC*]

In some embodiments, L is

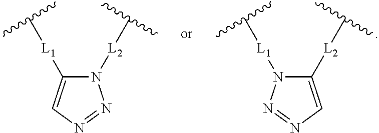

In some embodiments, L is

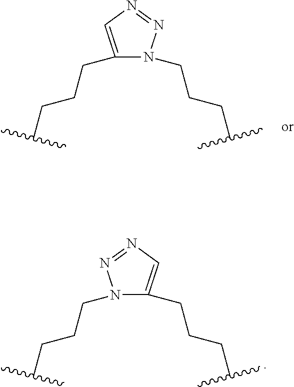 [SMILES: *CCCc1cnnn1CCC* ; *CCCc1cnnn1CCC*]

In some embodiments, R₁ and R₂ are H. In some embodiments, R₁ and R₂ are independently alkyl. In some embodiments, R₁ and R₂ are methyl. In some embodiments, the peptidomimetic macrocycle has the Formula (Ia):

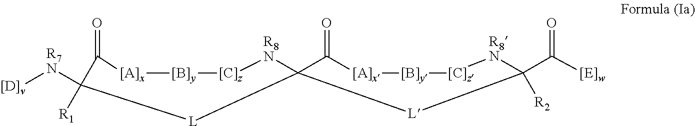

wherein: R₈′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a E residue; and x′, y′ and z′ are independently integers from 0-10.

In some embodiments, u is 2. In some embodiments, the peptidomimetic macrocycle has the Formula (Ib):

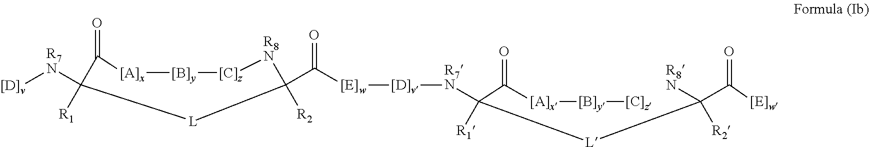

wherein: R₇′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue; R₈′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue; v′ and w′ are independently integers from 0-100; and x′, y′ and z′ are independently integers from 0-10, for example x′+y′+z′ is 2, 3, 6 or 10. In some embodiments, the sum of x+y+z is 2, 3 or 6, for example 3 or 6. In some embodiments, the sum of x′+y′+z′ is 2, 3 or 6, for example 3 or 6. In some embodiments, each of v and w is independently an integer from 1-10, 1-15, 1-20, or 1-25.

In some embodiments, u is 3. In some embodiments, the peptidomimetic macrocycle has the Formula (Ic):

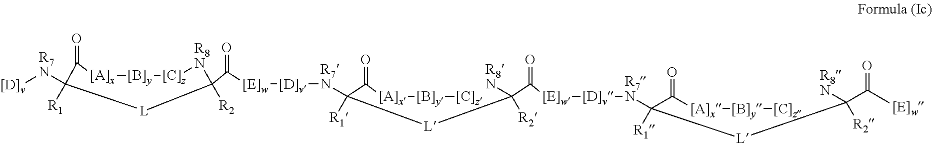

R₇″ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue; R₈″ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue; v″ and w″ are independently integers from 0-100; and x″, y″ and z″ are independently integers from 0-10, for example x″+y″+z″ is 2, 3, 6 or 10. In some embodiments, the peptidomimetic macrocycle has the Formula (IIIa) or Formula (IIIb):

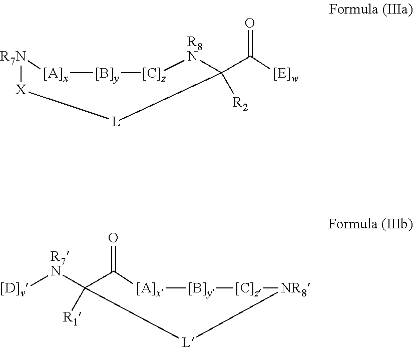

wherein: each A, C, D and E is independently an amino acid;
each B is independently an amino acid,

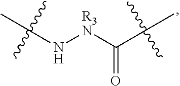

[—NH-L₃-CO—], [—NH-L₃-SO₂—], or [—NH-L₃-]; R₁′ and R₂ are independently —H, alkyl, alkenyl, alkynyl, arylalkyl, cycloalkyl, cycloalkylalkyl, heteroalkyl, or heterocycloalkyl, unsubstituted or substituted with halo-; or R₂ forms a macrocycle-forming linker L′ connected to the alpha position of one of said E amino acids;
R₃ is —H, alkyl, alkenyl, alkynyl, arylalkyl, heteroalkyl, cycloalkyl, heterocycloalkyl, cycloalkylalkyl, aryl, or heteroaryl, optionally substituted with R₅;
L and L′ are independently a macrocycle forming linker of the formula -L₁-L₂-,

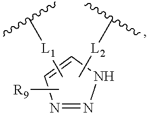

### or -L1-S-L2-S-L3-;

L₁, L₂ and L₃ are independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R₄ is independently alkylene, alkenylene, alkynylene, heteroalkylene, cycloalkylene, heterocycloalkylene, arylene, or heteroarylene;
each K is O, S, SO, SO₂, CO, CO₂, or CONR₃;
   each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
   R₇ or R₇′ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
   R₈ or R₈′ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
   R₉ is alkyl, alkenyl, alkynyl, aryl, cycloalkyl, cycloalkenyl, heteroaryl, or heterocyclyl group, unsubstituted or optionally substituted with Rₐ and/or R_{b};
   Rₐ and R_{b} are independently alkyl, OCH₃, CF₃, NH₂, CH₂NH₂, F, Br, I,

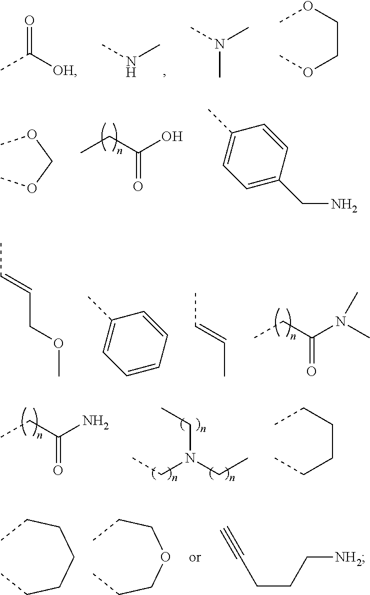

v and w′ are independently integers from 0-1000, for example 0-500, 0-200, 0-100, 0-50, 0-30, 0-20, or 0-10;
x, y, z, x′, y′ and z′ are independently integers from 0-10, for example the sum of x+y+z is 2, 3, 6 or 9, or the sum of x′+y′+z′ is 2, 3, 6, or 9;
n is an integer from 1-5;

### X is C═O, CHR_{c}, or C═S;

R_{c} is alkyl, alkenyl, alkynyl, arylalkyl, cycloalkyl, cycloalkylalkyl, heteroalkyl, or heterocycloalkyl; and
A, B, C, and E, taken together with the crosslinked amino acids connected by the macrocycle-forming linker -L₁-L₂-, form an amino acid sequence of the peptidomimetic macrocycle with at least about 60%, 65%, 70%, 75%, 80%, 85%, 90%, 95%, 96%, 97%, 98%, 99%, or 100% sequence identity to a sequence of Table 1a, 1b, 2a, or 2b. In some embodiments, the amino acid sequence of the peptidomimetic macrocycle has at least about 60%, 65%, 70%, 75%, 80%, 85%, 90%, 95%, 96%, 97%, 98%, 99%, or 100% sequence identity to a sequence of Table 1a or 3a. In some embodiments, the peptidomimetic macrocycle has the Formula (IIIc), (IIId), (IIIe), (IIIf) or (IIIg):

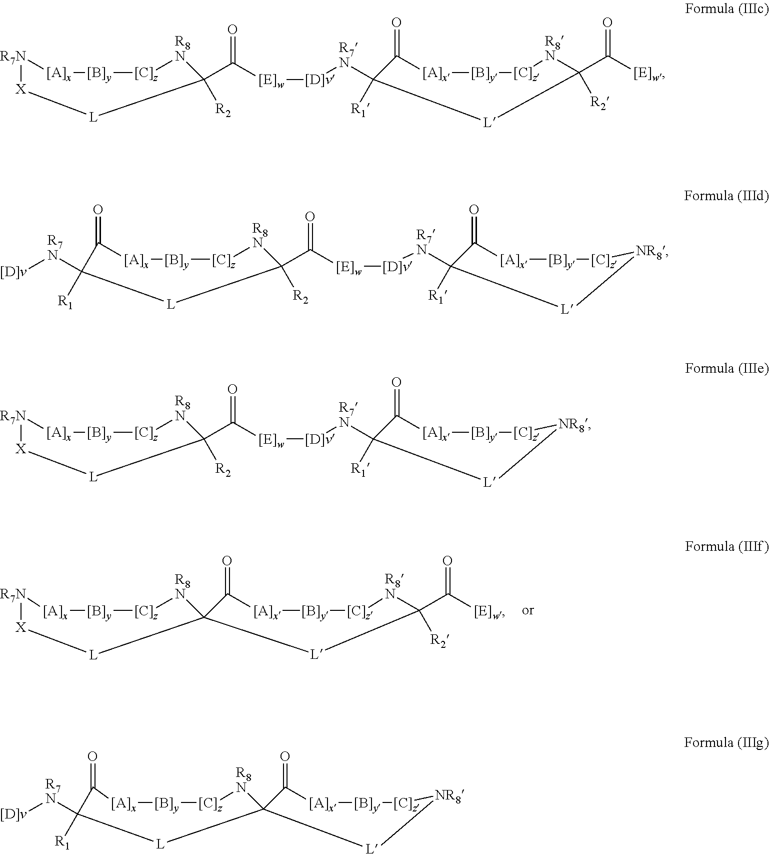

wherein R₁′ and R₂′ are independently —H, alkyl, alkenyl, alkynyl, arylalkyl, cycloalkyl, cycloalkylalkyl, heteroalkyl, or heterocycloalkyl, unsubstituted or substituted with halo-; and v, w, v′ and w′ are independently an integer from 0-100. In some embodiments, L₁ and L₂ are independently alkylene, alkenylene or alkynylene.

In one aspect, a composition is provided comprising a peptidomimetic macrocycle comprising an amino acid sequence of formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH, or a C-terminal capping group; X₁-X₃₆ are absent or are amino acids, such that at least X₇-X₂₂ are not absent; at least three, four, five, six, or seven amino acids from the group consisting of X₂₀, X₂₃, X₂₄, X₂₅, X₂₇, X₂₈, X₃₁, X₃₂, and X₃₄ are selected as follows: X₂₀ is Arg, X₂₃ is Trp or Phe, X₂₄ is Leu, X₂₅ is Arg, X₂₇ is Lys or Leu, X₂₈ is Leu or Ile, X₃₁ is Val or Ile, X₃₂ is His, and X₃₄ is Phe; and wherein the peptidomimetic macrocycle comprises at least one pair of crosslinked amino acids selected from the group consisting of amino acids X₁-X₃₆.

In one aspect, a composition is provided comprising a peptidomimetic macrocycle comprising an amino acid sequence of formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
   wherein: X₀ is —H or an N-terminal capping group; X₃₇ is —OH, or a C-terminal capping group; X₁-X₃₆ are absent or are amino acids, such that at least X₇-X₂₂ are not absent; at least three, four, five, six, or seven amino acids from the group consisting of X₂₀, X₂₃, X₂₄, X₂₅, X₂₇, X₂₈, X₃₁, X₃₂, and X₃₄ are selected as follows: X₂₀ is Arg or Cit or an analog thereof, X₂₃ is Trp or Phe or Ala or 1Nal or 2Nal, X₂₄ is Leu or Cpg or Cba or Ala or an analog thereof or a crosslinked amino acid, X₂₅ is Arg or His or Aib or Phe or Ser or Glu or Ala or Tyr or Trp or an analog thereof or a crosslinked amino acid, X₂₇ is Lys or Leu or Cit or Nle or hF or Tyr or His or Phe or Gln or an analog thereof or a crosslinked amino acid, X₂₈ is Leu or Ile or Cpg or Cba or Cha or an analog thereof or a crosslinked amino acid, X₃₁ is Val or Ile or Cpg or Cba or Nle or Thr or an analog thereof or a crosslinked amino acid, X₃₂ is His or Tyr or Phe or Ala or 2Pal or an analog thereof or a crosslinked amino acid, and X₃₄ is Phe or Tyr or Ala; and the peptidomimetic macrocycle comprises at least one pair of crosslinked amino acids selected from the group consisting of amino acids X₁-X₃₆.

In some embodiments, X₀ is —H or an N-terminal capping group, for example acetyl, 1NaAc, 2NaAc, PhAc, a fatty acid, a urea, a sulfonamide, or a polyalkylene oxide linked to the N-terminus of residue X₁; X₁ is Ser, Ala, Deg, Har, a dialkylated amino acid, Aib, Ac5c, Ac3c, Ac6c, desamino-Ser, desamino-Ac5c, desamino-Aib, Val, an analog thereof, or absent; X₂ is an aromatic amino acid, Val, Trp, Arg, D-Trp, D-Arg, F4COOH, Bip, F4NH₂, 1Nal, 2Nal, 2Pal, 3Pal, 4Pal, Bpa, Deg, Ile, an analog thereof, or absent; X₃ is Ser, Deg, Aib, Ac3c, Ac5c, Ac6c, Glu, Lys, Phe, Aib, Gly, Ala, an analog thereof, or absent; X₄ is Glu, Gln, Phe, His, an analog thereof, or absent; X₅ is Ile, His, Lys, Glu, Phe, an analog thereof, or absent; X₆ is Gln, Lys, Glu, Phe, Ala, an analog thereof, or absent; X₇ is an aromatic amino acid, a hydrophobic amino acid, Leu, Lys, Glu, Ala, Phe, Met, F4Cl, 1Nal, 2Nal, 2Pal, 3Pal, 4Pal, Phe, Nle, an analog thereof, or a crosslinked amino acid; X₈ is a hydrophobic amino acid, Met, Leu, Nle, an analog thereof, or a crosslinked amino acid; X₉ is an aromatic amino acid, His, Aib, or an analog thereof; X₁₀ is Asn, Asp, Gln, Ala, Ser, Val, His, Trp, Aib, an analog thereof, or a crosslinked amino acid; X₁₁ is a hydrophobic amino acid, a positively charged amino acid, an aromatic amino acid, Leu, Lys, Har, Arg, Ala, Val, Ile, Met, Phe, Trp, D-Trp, Nle, Cit, hK, hL, an analog thereof, or a crosslinked amino acid; X₁₂ is a D-amino acid, a hydrophobic amino acid, a hydrophilic amino acid, an aromatic amino acid, a positively charged amino acid, a negatively charged amino acid, an uncharged amino acid, Gly, D-Trp, Ala, Aib, Arg, His, Trp, an analog thereof, or a crosslinked amino acid; X₁₃ is a positively charged amino acid, Lys, Ser, Ala, Aib, Leu, Glu, Gln, Arg, His, Phe, Trp, Pro, Cit, Kfam, Ktam, an analog thereof, or a crosslinked amino acid; X₁₄ is an aromatic amino acid, His, Ser, Trp, Ala, Leu, Lys, Arg, Phe, Trp, Aib, an analog thereof, or a crosslinked amino acid; X₁₅ is a hydrophobic amino acid, Leu, Ile, Tyr, Aib, an analog thereof, or a crosslinked amino acid; X₁₆ is Asn, Gln, Lys, Ala, Glu, an analog thereof, or a crosslinked amino acid; X₁₇ is Ser, Asp, β-Ala, β-hPhe, Aib, an analog thereof, or a crosslinked amino acid; X₁₈ is a hydrophobic amino acid, Met, Nle, Leu, β-hIle, hSer(OMe), β-hPhe, Aib, an analog thereof, or a crosslinked amino acid; X₁₉ is a positively charged amino acid, Glu, Arg, Ser, Aib, Cit, Glu, Ala, an analog thereof, or a crosslinked amino acid; X₂₀ is a positively charged amino acid, Cit, Arg, Ala, an analog thereof, or a crosslinked amino acid; X₂₁ is a positively charged amino acid, Cit, Val, Arg, Lys, Gln, Cit, Ala, an analog thereof, or a crosslinked amino acid; X₂₂ is an aromatic amino acid, Glu, Phe, Ser, Aib, an analog thereof, or a crosslinked amino acid; X₂₃ is an aromatic amino acid, a hydrophobic amino acid, Trp, Phe, Ala, 9-Aal, 1Nal, 2Nal, an analog thereof, absent, or a crosslinked amino acid; X₂₄ is an aromatic amino acid, a hydrophobic amino acid, Leu, Ala, Cba, Cpg, Aib, an analog thereof, absent, or a crosslinked amino acid; X₂₅ is a positively charged amino acid, Cit, Arg, His, Leu, Trp, Tyr, Phe, Ala, Ser, Glu, Aib, an analog thereof, absent, or a crosslinked amino acid; X₂₆ is a positively charged amino acid, Lys, His, Ala, Phe, Ser, Glu, AmO, AmK, Cit, and Aib an analog thereof, absent, or a crosslinked amino acid; X₂₇ is a positively charged amino acid, Cit, Lys, Leu, Arg, Nle, Tyr, His, Phe, hF, Leu, Gln, an analog thereof, absent, or a crosslinked amino acid; X₂₈ is an aromatic amino acid, a hydrophobic amino acid, Leu, Ile, Cba, Cha, Cpg, Aib, an analog thereof, absent, or a crosslinked amino acid; X₂₉ is Gln, Ala, Glu, Ser, Aib, an analog thereof, absent, or a crosslinked amino acid; X₃₀ is Asp, Glu, Leu, Arg, hPhe, Asn, His, Ser, Ala, Phe, an analog thereof, absent, or a crosslinked amino acid; X₃₁ is an aromatic amino acid, a hydrophobic amino acid, Val, Ile, Nle, Thr, Ser, Cba, Cpg, an analog thereof, absent, or a crosslinked amino acid; X₃₂ is an aromatic amino acid, His, Trp, Arg, Phe, Tyr, Ile, Ala, 2Pal, 3Pal, 4Pal, an analog thereof, absent, or a crosslinked amino acid; X₃₃ is Asn, Thr, Glu, Asp, Lys, Phe, an analog thereof, absent, or a crosslinked amino acid; X₃₄ is an aromatic amino acid, a hydrophobic amino acid, Phe, Ala, Tyr, Arg, 2Nal, hF, Glu, Lys, Ser, an analog thereof, absent, or a crosslinked amino acid; X₃₅ is Glu, Gly, an analog thereof, absent, or a crosslinked amino acid; X₃₆ is an aromatic amino acid, Tyr, Pra, an analog thereof, absent, or a crosslinked amino acid; and X₃₇ is —OH, or a C-terminal capping group, for example a primary, secondary, or tertiary amino group, an alkyloxy or an aryloxy group.

In some embodiments, X₀ is —H or an N-terminal capping group, for example acetyl, 1NaAc, 2NaAc, PhAc, a fatty acid, a urea, a sulfonamide, or a polyalkylene oxide linked to the N-terminus of residue X₁; X₁ is Ser, Ala, Deg, Har, a dialkylated amino acid, Aib, Ac5c, Ac3c, Ac6c, desamino-Ser, desamino-Ac5c, desamino-Aib, Val, an analog thereof, or absent; X₂ is an aromatic amino acid, Val, Trp, Arg, D-Trp, D-Arg, F4COOH, Bip, F4NH₂, 1Nal, 2Nal, 2Pal, 3Pal, 4Pal, Bpa, Deg, Ile, an analog thereof, or absent; X₃ is Ser, Deg, Aib, Ac3c, Ac5c, Ac6c, Glu, Lys, Phe, Aib, Gly, Ala, an analog thereof, or absent; X₄ is Glu, Gln, Phe, His, an analog thereof, or absent; X₅ is Ile, His, Lys, Glu, Phe, an analog thereof, or absent; X₆ is Gln, Lys, Glu, Phe, Ala, an analog thereof, or absent; X₇ is an aromatic amino acid, a hydrophobic amino acid, Leu, Lys, Glu, Ala, Phe, F4Cl, 1Nal, 2Nal, 2Pal, 3Pal, 4Pal, Phe, or an analog thereof; X₈ is a hydrophobic amino acid, Met, Leu, Nle, or an analog thereof; X₉ is an aromatic amino acid, His, or an analog thereof; X₁₀ is Asn, Asp, Gln, Ala, Ser, Val, His, Trp, an analog thereof, or a crosslinked amino acid; X₁₁ is a hydrophobic amino acid, a positively charged amino acid, an aromatic amino acid, Leu, Lys, Har, Arg, Ala, Val, Ile, Met, Phe, Trp, D-Trp or an analog thereof; X₁₂ is a D-amino acid, a hydrophobic amino acid, a hydrophilic amino acid, an aromatic amino acid, a positively charged amino acid, a negatively charged amino acid, an uncharged amino acid, Gly, D-Trp, Ala, Aib, Arg, His, Trp or an analog thereof; X₁₃ is a positively charged amino acid, Lys, Ser, Ala, Aib, Leu, Glu, Gln, Arg, His, Phe, Trp, Pro or an analog thereof; X₁₄ is an aromatic amino acid, His, Ser, Trp, Ala, Leu, Lys, Arg, Phe, Trp, an analog thereof, or a crosslinked amino acid; X₁₅ is a hydrophobic amino acid, Leu, Ile, Tyr, an analog thereof, or a crosslinked amino acid; X₁₆ is Asn, Gln, Lys, an analog thereof, or a crosslinked amino acid; X₁₇ is Ser, Asp, β-Ala, β-hPhe, an analog thereof, or a crosslinked amino acid; X₁₈ is a hydrophobic amino acid, Met, Nle, Leu, β-hIle, hSer(OMe), β-hPhe, an analog thereof, or a crosslinked amino acid; X₁₉ is a positively charged amino acid, Cit, Glu, Arg, Ser, an analog thereof, or a crosslinked amino acid; X₂₀ is a positively charged amino acid, Cit, Arg, an analog thereof, or a crosslinked amino acid; X₂₁ is a positively charged amino acid, Cit, Val, Arg, Lys, Gln, an analog thereof, or a crosslinked amino acid; X₂₂ is an aromatic amino acid, Glu, Phe, an analog thereof, or a crosslinked amino acid; X₂₃ is an aromatic amino acid, a hydrophobic amino acid, Trp, Phe, 9-Aal, 1Nal, 2Nal, an analog thereof, absent, or a crosslinked amino acid; X₂₄ is an aromatic amino acid, a hydrophobic amino acid, Leu, an analog thereof, absent, or a crosslinked amino acid; X₂₅ is a positively charged amino acid, Cit, Arg, His, Leu, Trp, Tyr, Phe, an analog thereof, absent, or a crosslinked amino acid; X₂₆ is a positively charged amino acid, Lys, His, an analog thereof, absent, or a crosslinked amino acid; X₂₇ is a positively charged amino acid, Cit, Lys, Leu, Arg, Nle, Tyr, His, Phe, hF, Leu, Gln, an analog thereof, absent, or a crosslinked amino acid; X₂₈ is an aromatic amino acid, a hydrophobic amino acid, Leu, Ile, an analog thereof, absent, or a crosslinked amino acid; X₂₉ is Gln, Ala, Glu, an analog thereof, absent, or a crosslinked amino acid; X₃₀ is Asp, Glu, Leu, Arg, hPhe, Asn, His, Ser, an analog thereof, absent, or a crosslinked amino acid; X₃₁ is an aromatic amino acid, a hydrophobic amino acid, Val, Ile, Nle, Thr, Ser, an analog thereof, absent, or a crosslinked amino acid; X₃₂ is an aromatic amino acid, His, Trp, Arg, Phe, Tyr, Ile, 2Pal, 3Pal, 4Pal, an analog thereof, absent, or a crosslinked amino acid; X₃₃ is Asn, Thr, Glu, Asp, Lys, an analog thereof, absent, or a crosslinked amino acid; X₃₄ is an aromatic amino acid, a hydrophobic amino acid, Phe, Ala, Tyr, Arg, 2Nal, hF, Glu, Lys, an analog thereof, absent, or a crosslinked amino acid; X₃₅ is Glu, an analog thereof, absent, or a crosslinked amino acid; X₃₆ is an aromatic amino acid, Tyr, an analog thereof, absent, or a crosslinked amino acid; and X₃₇ is —OH, or a C-terminal capping group, for example a primary, secondary, or tertiary amino group, an alkyloxy or an aryloxy group.

In some embodiments, X₉ and X₁₃ are crosslinked amino acids. In some embodiments, X₁₀ and X₁₄ are crosslinked amino acids. In some embodiments, X₁₁ and X₁₅ are crosslinked amino acids. In some embodiments, X₁₂ and X₁₆ are crosslinked amino acids. In some embodiments, X₁₃ and X₁₇ are crosslinked amino acids. In some embodiments, X₁₄ and X₁₈ are crosslinked amino acids. In some embodiments, X₁₈ and X₂₂ are crosslinked amino acids. In some embodiments, X₂₂ and X₂₆ are crosslinked amino acids. In some embodiments, X₂₄ and X₂₈ are crosslinked amino acids. In some embodiments, X₂₆ and X₃₀ are crosslinked amino acids. In some embodiments, X₂₇ and X₃₁ are crosslinked amino acids. In some embodiments, the peptidomimetic macrocycle comprises two pairs of crosslinked amino acids. In some embodiments, X₁₄ and X₁₈ are crosslinked amino acids, and X₂₆ and X₃₀ are crosslinked amino acids. In some embodiments, X₁₄ and X₁₈ are crosslinked amino acids, and X₂₂ and X₂₆ are crosslinked amino acids. In some embodiments, X₁₄ and X₁₈ are crosslinked amino acids, and X₂₄ and X₂₈ are crosslinked amino acids. In some embodiments, X₁₄ and X₁₈ are crosslinked amino acids, and X₂₇ and X₃₁ are crosslinked amino acids. In some embodiments, X₁₃ and X₁₇ are crosslinked amino acids, and X₂₆ and X₃₀ are crosslinked amino acids.

In some embodiments, X₁-X₆ are absent. In some embodiments, X₃₅-X₃₆ are absent.

In some embodiments, X₁₁ is Har. In some embodiments, X₁₁ is Leu. In some embodiments, X₁₉ is a positively charged amino acid, Cit, Arg. or an analog thereof. In some embodiments, X₁₉ is Arg. In some embodiments, X₂₃ is Trp. In some embodiments, X₂₃ is Phe. In some embodiments, X₂₄ is Leu. In some embodiments, X₂₅ is Arg. In some embodiments, X₂₇ is Lys. In some embodiments, X₂₇ is Leu. In some embodiments, X₂₈ is Leu. In some embodiments, X₂₈ is Ile. In some embodiments, X₃₁ is Val. In some embodiments, X₃₁ is Ile. In some embodiments, X₃₂ is His. In some embodiments, X₃₄ is Phe. In some embodiments, X₂₀ is a positively charged amino acid, Cit, Arg, or an analog thereof. In some embodiments, X₂₀ is Arg. In some embodiments, X₂₁ is a positively charged amino acid, Cit, Arg, Lys, or an analog thereof. In some embodiments, X₂₁ is Arg. In some embodiments, X₂₀ is Arg, X₂₃ is Trp, X₂₄ is Leu, X₂₅ is Arg, X₂₇ is Lys, X₂₈ is Leu, X₃₁ is Val, and X₃₄ is Phe. In some embodiments, X₂₀ is Arg, X₂₃ is Phe, X₂₄ is Leu, X₂₇ is Leu, X₂₈ is Ile, X₃₁ is Ile, and X₃₂ is His.

In one aspect, a composition is provided comprising a peptidomimetic macrocycle having the Formula: [A-B-C] wherein: A is an amino acid sequence comprising at least three amino acids selected from PTH (7-14); B is an amino acid sequence comprising at least three amino acids selected from PTHrP (15-21); and C is an amino acid sequence comprising at least six amino acids selected from PTH (22-34); wherein the peptidomimetic macrocycle comprises at least one macrocycle-forming linker.

In some embodiments, A is X₇-X₈-X₉-X₁₀-X₁₁-X₁₂-X₁₃-X₁₄; B is X₁₅-X₁₆-X₁₇-X₁₈-X₁₉-X₂₀-X₂₁; C is X₂₂-X₂₃-X₂₄-X₂₅-X₂₆-X₂₇-X₂₈-X₂₉-X₃₀-X₃₁-X₃₂-X₃₃-X₃₄-; X₀ is —H or an N-terminal capping group; X₁-X₆ are absent or are amino acids; X₃₇ is —OH, or a C-terminal capping group; and X₃₅-X₃₆ are absent or are amino acids.

In some embodiments, the peptidomimetic macrocycle comprises at least one macrocycle-forming linker connecting a pair of amino acids selected from the group consisting of amino acids X₇-X₃₄. In some embodiments, the macrocycle-forming linker connects amino acids X₉ and X₁₃. In some embodiments, the macrocycle-forming linker connects amino acids X₁₃ and X₁₇. In some embodiments, the macrocycle-forming linker connects amino acids X₁₈ and X₂₂. In some embodiments, the macrocycle-forming linker connects amino acids X₂₄ and X₂₈.

In some embodiments, X₁₉ is a positively charged amino acid, Cit, Arg. or an analog thereof. In some embodiments, X₁₉ is Arg. In some embodiments, X₂₀ is a positively charged amino acid, Cit, Arg, or an analog thereof. In some embodiments, X₂₀ is Arg. In some embodiments, X₂₁ is a positively charged amino acid, Cit, Arg, Lys, or an analog thereof. In some embodiments, X₂₁ is Arg.

A composition is provided comprising a peptidomimetic macrocycle selected from Table 3. A composition is provided comprising a peptidomimetic macrocycle selected from Table 7. A composition is provided comprising a peptidomimetic macrocycle selected from Table 6. A composition is provided comprising a peptidomimetic macrocycle selected from Table 8.

In some embodiments, a peptidomimetic macrocycle comprises a helix. In some embodiments, a peptidomimetic macrocycle comprises an α-helix. In some embodiments, a peptidomimetic macrocycle comprises an α,α-disubstituted amino acid. In some embodiments, each amino acid connected by the macrocycle-forming linker is an α,α-disubstituted amino acid.

### Preparation of Peptidomimetic Macrocycles

Peptidomimetic macrocycles provided herein may be prepared by any of a variety of methods known in the art. For example, any of the cross-linked amino acids in Tables 1, 2, and 3 may be substituted with a residue capable of forming a crosslinker with a second residue in the same molecule or a precursor of such a residue.

Various methods to effect formation of peptidomimetic macrocycles are known in the art. For example, the preparation of peptidomimetic macrocycles of Formula (I) is described in Schafmeister et al., J. Am. Chem. Soc. 122:5891-5892 (2000); Schafmeister & Verdine, J. Am. Chem. Soc. 122:5891 (2005); Walensky et al., Science 305:1466-1470 (2004); U.S. Pat. No. 7,192,713 and PCT application WO 2008/121767. The α,α-disubstituted amino acids and amino acid precursors disclosed in the cited references may be employed in synthesis of the peptidomimetic macrocycle precursor polypeptides. For example, the “S5-olefin amino acid” is (S)-α-(2′-pentenyl) alanine and the “R8 olefin amino acid” is (R)-α-(2′-octenyl) alanine. Following incorporation of such amino acids into precursor polypeptides, the terminal olefins are reacted with a metathesis catalyst, leading to the formation of the peptidomimetic macrocycle. In various embodiments, the following amino acids may be employed in the synthesis of the peptidomimetic macrocycle:

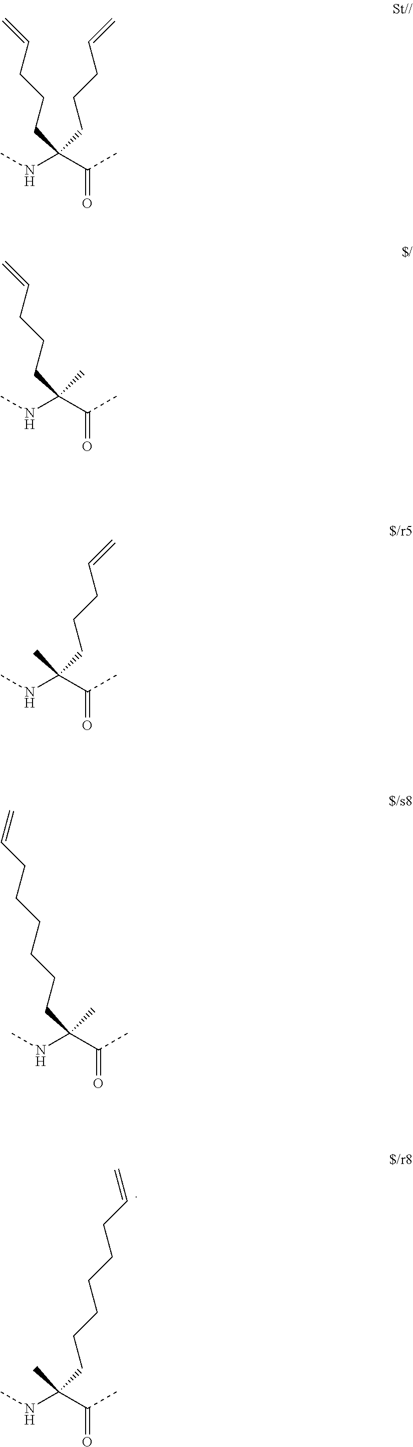

In some embodiments, x+y+z is 3, and A, B and C are independently natural or non-natural amino acids. In other embodiments, x+y+z is 6, and A, B and C are independently natural or non-natural amino acids.

In some embodiments, the contacting step is performed in a solvent selected from the group consisting of protic solvent, aqueous solvent, organic solvent, and mixtures thereof. For example, the solvent may be chosen from the group consisting of H₂O, THF, THF/H₂O, tBuOH/H₂O, DMF, DIPEA, CH₃CN or CH₂Cl₂, ClCH₂CH₂Cl or a mixture thereof. The solvent may be a solvent which favors helix formation.

Alternative but equivalent protecting groups, leaving groups or reagents are substituted, and certain of the synthetic steps are performed in alternative sequences or orders to produce the desired compounds. Synthetic chemistry transformations and protecting group methodologies (protection and deprotection) useful in synthesizing the compounds described herein include, e.g., those such as described in Larock, “Comprehensive Organic Transformations”, VCH Publishers (1989); Greene and Wuts, “Protective Groups in Organic Synthesis,” 2d. Ed., John Wiley and Sons (1991); Fieser and Fieser, Fieser and Fieser's Reagents for Organic Synthesis,” John Wiley and Sons (1994); Paquette, ed., Encyclopedia of Reagents for Organic Synthesis,” John Wiley and Sons (1995), and subsequent editions thereof.

The peptidomimetic macrocycles provided herein are made, e.g., by chemical synthesis methods, such as described in Fields et al., Chapter 3 in “Synthetic Peptides: A User's Guide,” ed. Grant, W. H. Freeman & Co., New York, N.Y., 1992, p. 77. Hence, e.g., peptides are synthesized using the automated Merrifield techniques of solid phase synthesis with the amine protected by either tBoc or Fmoc chemistry using side chain protected amino acids on, e.g., an automated peptide synthesizer (e.g., Applied Biosystems (Foster City, Calif.), Model 430A, 431, or 433).

One manner of producing the peptidomimetic precursors and peptidomimetic macrocycles described herein uses solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Side chain functional groups are protected as necessary with base stable, acid labile groups.

Longer peptidomimetic precursors are produced, e.g., by conjoining individual synthetic peptides using native chemical ligation. Alternatively, the longer synthetic peptides are biosynthesized by well-known recombinant DNA and protein expression techniques. Such techniques are provided in well-known standard manuals with detailed protocols. To construct a gene encoding a peptidomimetic precursor of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptidomimetic precursors are made, e.g., in a high-throughput, combinatorial fashion using, e.g., a high-throughput polychannel combinatorial synthesizer (e.g., Thuramed TETRAS multichannel peptide synthesizer from CreoSalus, Louisville, Ky. or Model Apex 396 multichannel peptide synthesizer from amino acidPPTEC, Inc., Louisville, Ky.).

In some embodiments, the peptidomimetic macrocycles comprise triazole macrocycle-forming linkers. For example, the synthesis of such peptidomimetic macrocycles involves a multi-step process that features the synthesis of a peptidomimetic precursor containing an azide moiety and an alkyne moiety; followed by contacting the peptidomimetic precursor with a macrocyclization reagent to generate a triazole-linked peptidomimetic macrocycle. Such a process is described, e.g., in U.S. application Ser. No. 12/037,041, filed on Feb. 25, 2008. Macrocycles or macrocycle precursors are synthesized, e.g., by solution phase or solid-phase methods, and can contain both naturally-occurring and non-naturally-occurring amino acids. See, e.g., Hunt, “The Non-Protein Amino Acids” in “Chemistry and Biochemistry of the Amino Acids,” edited by G. C. Barrett, Chapman and Hall, 1985.

In some embodiments, an azide is linked to the α-carbon of a residue and an alkyne is attached to the α-carbon of another residue. In some embodiments, the azide moieties are azido-analogs of amino acids L-lysine, D-lysine, alpha-methyl-L-lysine, alpha-methyl-D-lysine, L-ornithine, D-ornithine, alpha-methyl-L-ornithine or alpha-methyl-D-ornithine. In another embodiment, the alkyne moiety is L-propargylglycine. In yet other embodiments, the alkyne moiety is an amino acid selected from the group consisting of L-propargylglycine, D-propargylglycine, (S)-2-amino-2-methyl-4-pentynoic acid, (R)-2-amino-2-methyl-4-pentynoic acid, (S)-2-amino-2-methyl-5-hexynoic acid, (R)-2-amino-2-methyl-5-hexynoic acid, (S)-2-amino-2-methyl-6-heptynoic acid, (R)-2-amino-2-methyl-6-heptynoic acid, (S)-2-amino-2-methyl-7-octynoic acid, (R)-2-amino-2-methyl-7-octynoic acid, (S)-2-amino-2-methyl-8-nonynoic acid and (R)-2-amino-2-methyl-8-nonynoic acid.

The following synthetic schemes are provided solely to illustrate the present invention and are not intended to limit the scope of the invention, as described herein. To simplify the drawings, the illustrative schemes depict azido amino acid analogs □-azido-α-methyl-L-lysine and □-azido-α-methyl-D-lysine, and alkyne amino acid analogs L-propargylglycine, (S)-2-amino-2-methyl-4-pentynoic acid, and (S)-2-amino-2-methyl-6-heptynoic acid. Thus, in the following synthetic schemes, each R₁, R₂, R₇ and R₈ is —H; each L₁ is —(CH₂)₄—; and each L₂ is —(CH₂)—. However, as noted throughout the detailed description above, many other amino acid analogs can be employed in which R₁, R₂, R₇, R₈, L₁ and L₂ can be independently selected from the various structures disclosed herein.

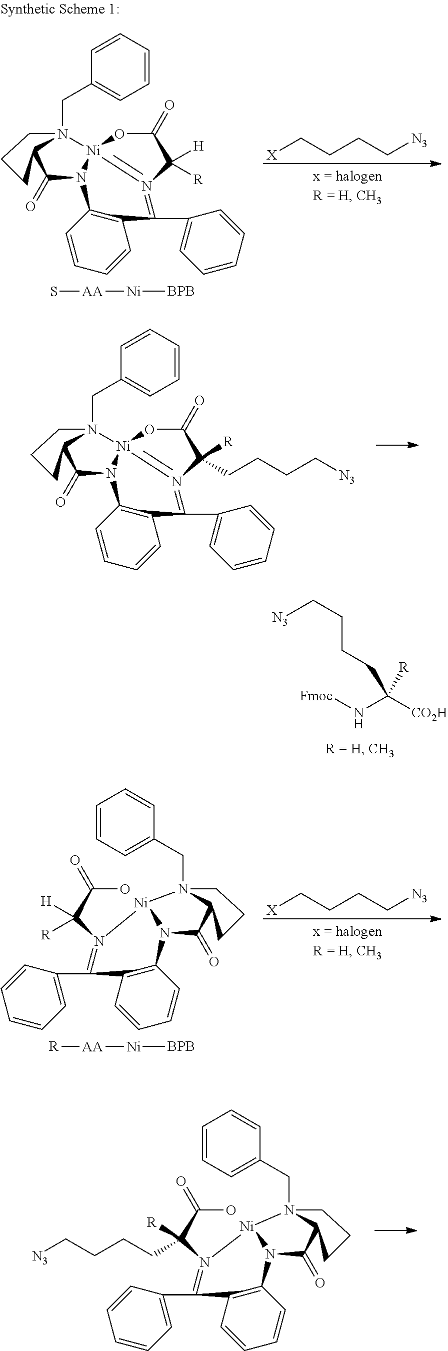

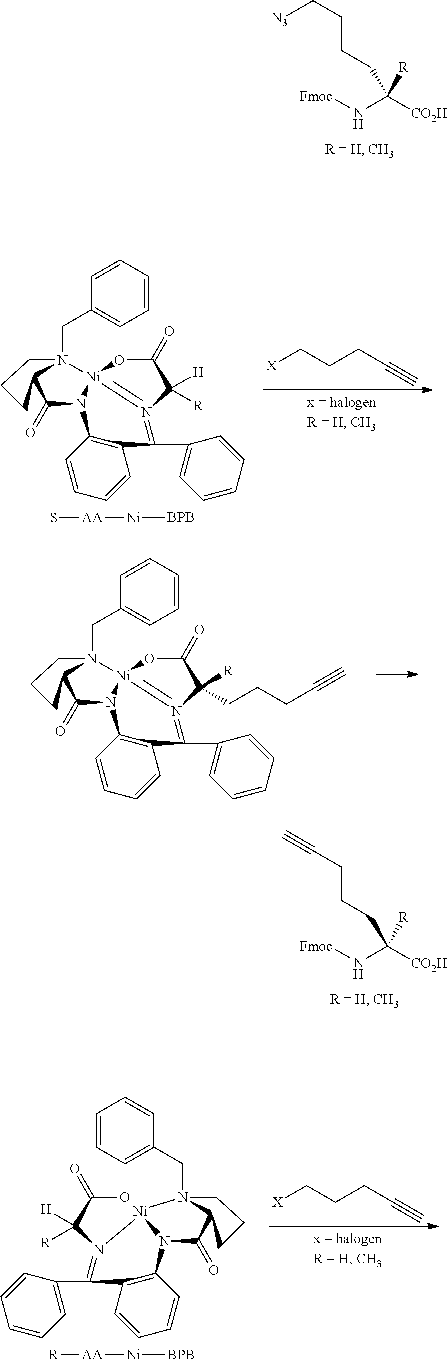

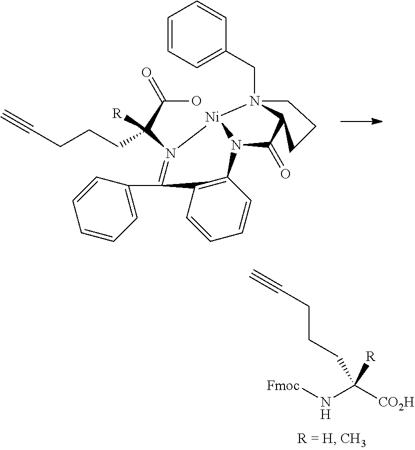

Synthetic Scheme 1 describes the preparation of several compounds of the invention. Ni(II) complexes of Schiff bases derived from the chiral auxiliary (S)-2-[N—(N′-benzylprolyl)amino]benzophenone (BPB) and amino acids such as glycine or alanine are prepared as described in Belokon et al. (1998), Tetrahedron Asymm. 9:4249-4252. The resulting complexes are subsequently reacted with alkylating reagents comprising an azido or alkynyl moiety to yield enantiomerically enriched compounds of the invention. If desired, the resulting compounds can be protected for use in peptide synthesis.

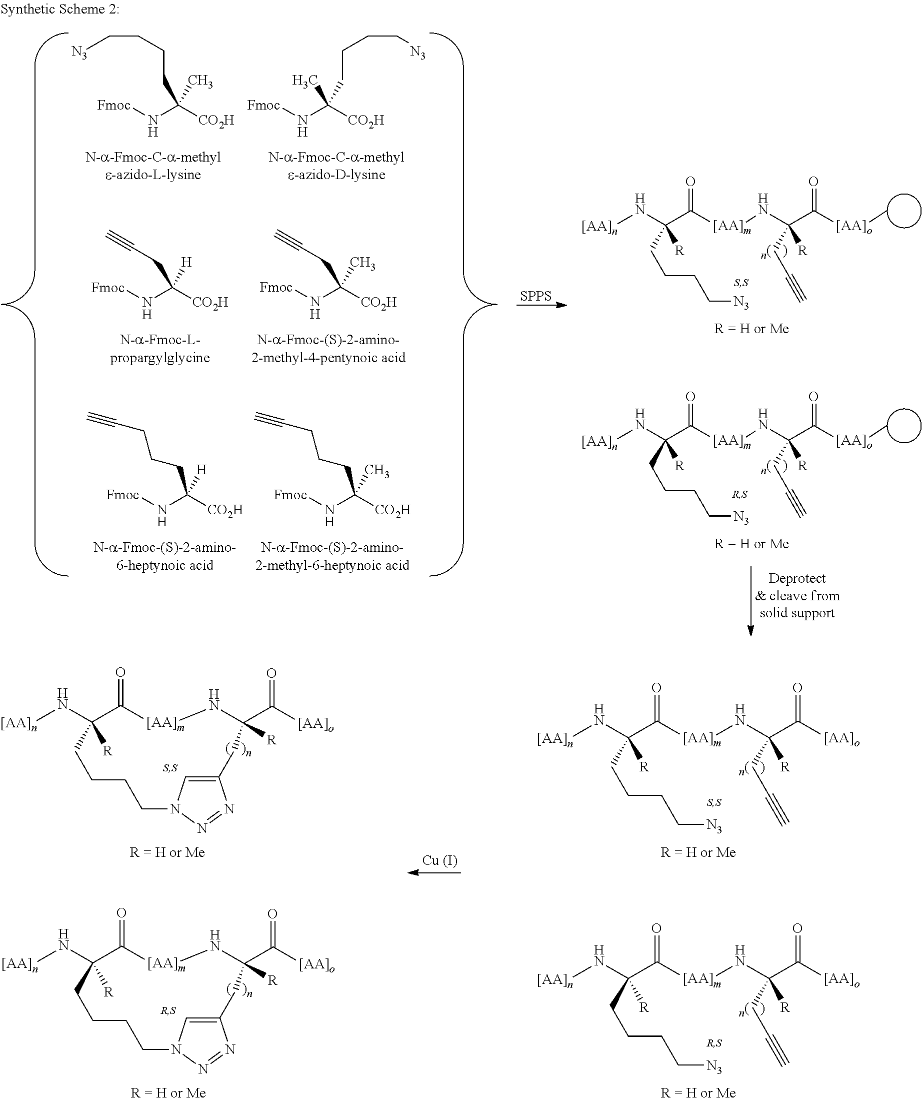

In the general method for the synthesis of peptidomimetic macrocycles shown in Synthetic Scheme 2, the peptidomimetic precursor contains an azide moiety and an alkyne moiety and is synthesized by solution-phase or solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-□-azido-L-lysine, and N-methyl-□-azido-D-lysine. The peptidomimetic precursor is then deprotected and cleaved from the solid-phase resin by standard conditions (e.g., strong acid such as 95% TFA). The peptidomimetic precursor is reacted as a crude mixture or is purified prior to reaction with a macrocyclization reagent such as a Cu(I) in organic or aqueous solutions (Rostovtsev et al. (2002), Angew. Chem. Int. Ed. 41:2596-2599; Tornoe et al. (2002), J. Org. Chem. 67:3057-3064; Deiters et al. (2003), J. Am. Chem. Soc. 125:11782-11783; Punna et al. (2005), Angew. Chem. Int. Ed. 44:2215-2220). In one embodiment, the triazole forming reaction is performed under conditions that favor α-helix formation. In one embodiment, the macrocyclization step is performed in a solvent chosen from the group consisting of H₂O, THF, CH₃CN, DMF, DIPEA, tBuOH or a mixture thereof. In another embodiment, the macrocyclization step is performed in DMF. In some embodiments, the macrocyclization step is performed in a buffered aqueous or partially aqueous solvent.

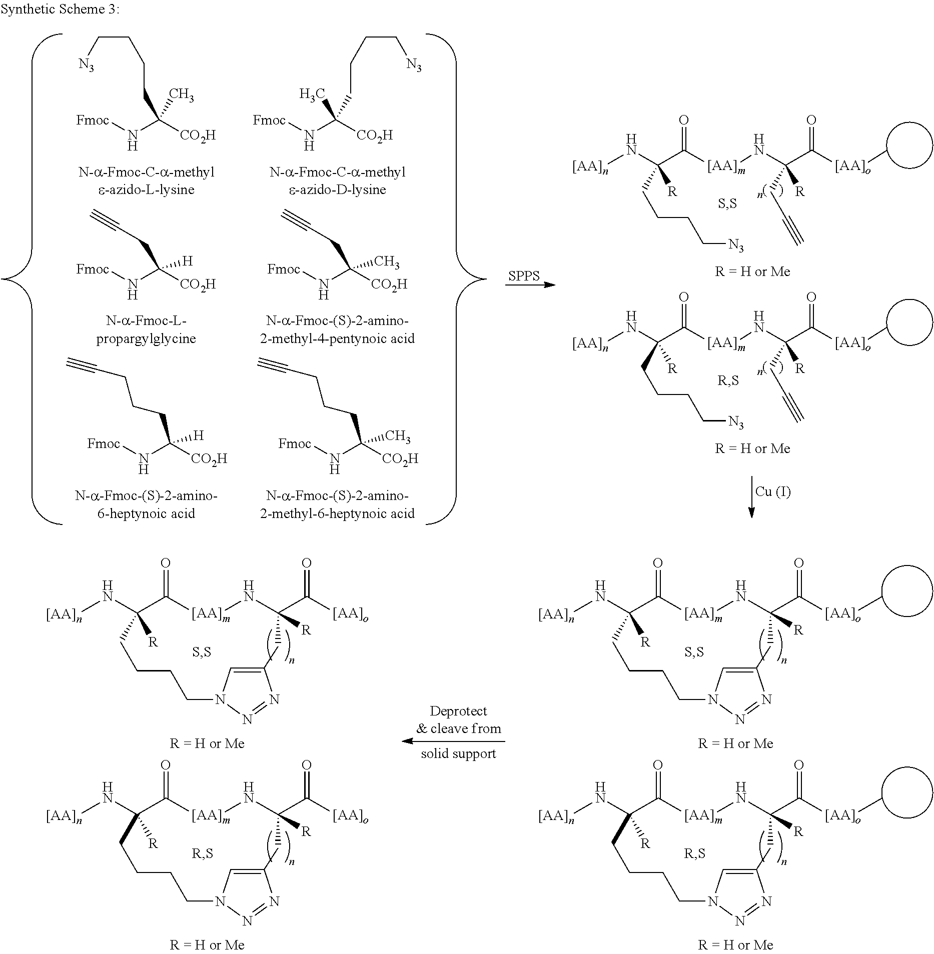

In the general method for the synthesis of peptidomimetic macrocycles shown in Synthetic Scheme 3, the peptidomimetic precursor contains an azide moiety and an alkyne moiety and is synthesized by solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-□-azido-L-lysine, and N-methyl-□-azido-D-lysine. The peptidomimetic precursor is reacted with a macrocyclization reagent such as a Cu(I) reagent on the resin as a crude mixture (Rostovtsev et al. (2002), Angew. Chem. Int. Ed. 41:2596-2599; Tornoe et al. (2002), J. Org. Chem. 67:3057-3064; Deiters et al. (2003), J. Am. Chem. Soc. 125:11782-11783; Punna et al. (2005), Angew. Chem. Int. Ed. 44:2215-2220). The resultant triazole-containing peptidomimetic macrocycle is then deprotected and cleaved from the solid-phase resin by standard conditions (e.g., strong acid such as 95% TFA). In some embodiments, the macrocyclization step is performed in a solvent chosen from the group consisting of CH₂Cl₂, ClCH₂CH₂Cl, DMF, THF, NMP, DIPEA, 2,6-lutidine, pyridine, DMSO, H₂O or a mixture thereof. In some embodiments, the macrocyclization step is performed in a buffered aqueous or partially aqueous solvent.

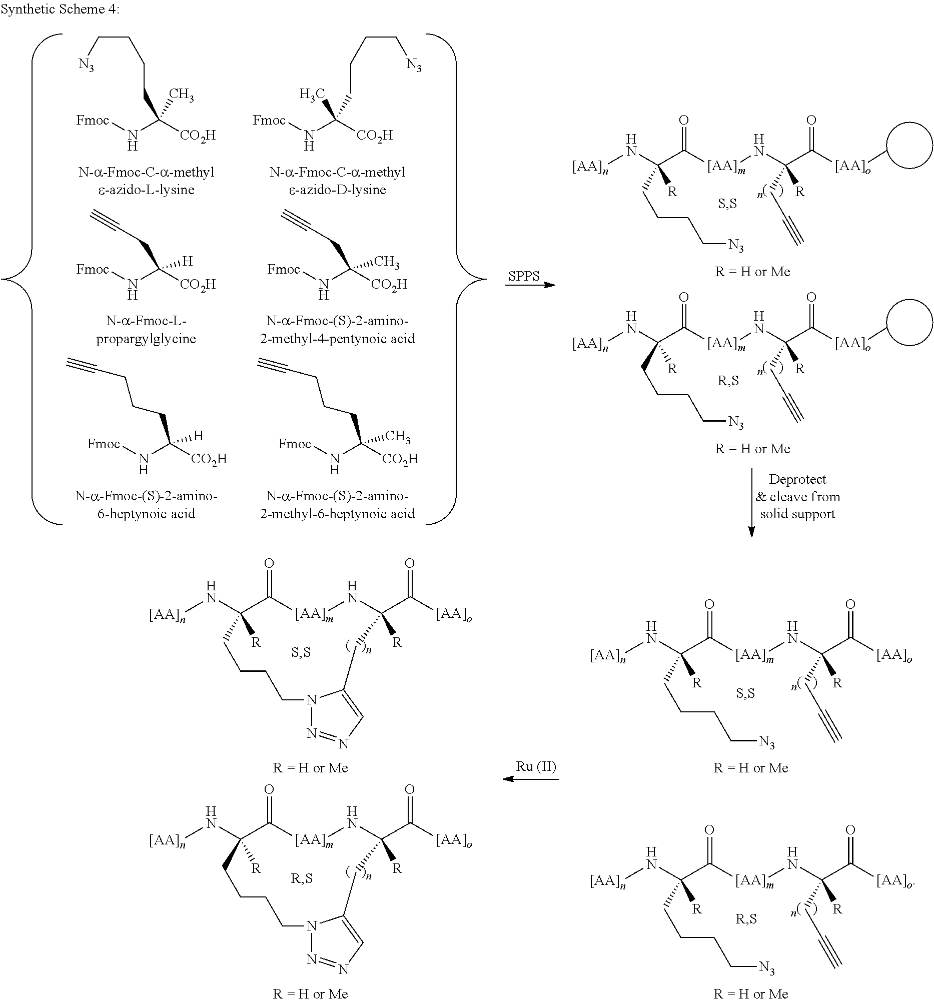

In the general method for the synthesis of peptidomimetic macrocycles shown in Synthetic Scheme 4, the peptidomimetic precursor contains an azide moiety and an alkyne moiety and is synthesized by solution-phase or solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-□-azido-L-lysine, and N-methyl-□-azido-D-lysine. The peptidomimetic precursor is then deprotected and cleaved from the solid-phase resin by standard conditions (e.g., strong acid such as 95% TFA). The peptidomimetic precursor is reacted as a crude mixture or is purified prior to reaction with a macrocyclization reagent such as a Ru(II) reagents, for example Cp*RuCl(PPh₃)₂ or [Cp*RuCl]₄ (Rasmussen et al. (2007), Org. Lett. 9:5337-5339; Zhang et al. (2005), J. Am. Chem. Soc. 127:15998-15999). In some embodiments, the macrocyclization step is performed in a solvent chosen from the group consisting of DMF, CH₃CN and THF.

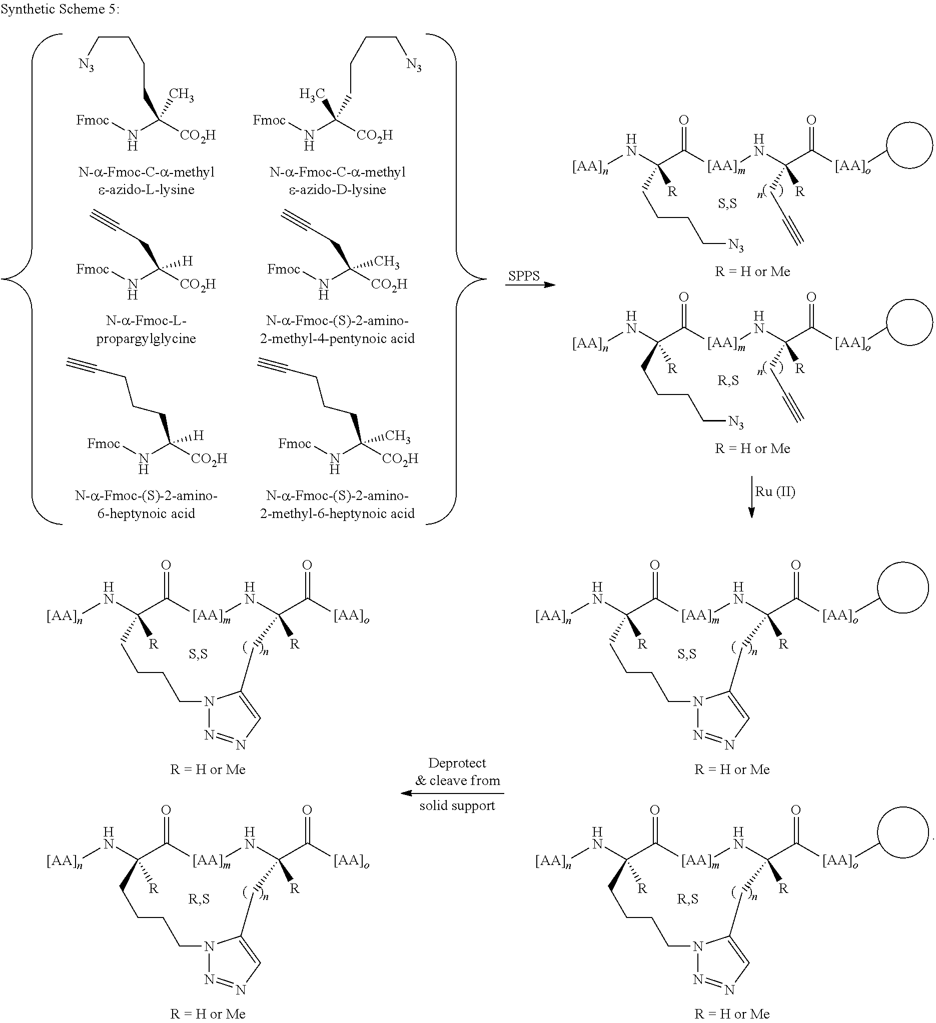

In the general method for the synthesis of peptidomimetic macrocycles shown in Synthetic Scheme 5, the peptidomimetic precursor contains an azide moiety and an alkyne moiety and is synthesized by solid-phase peptide synthesis (SPPS) using the commercially available amino acid N-α-Fmoc-L-propargylglycine and the N-α-Fmoc-protected forms of the amino acids (S)-2-amino-2-methyl-4-pentynoic acid, (S)-2-amino-6-heptynoic acid, (S)-2-amino-2-methyl-6-heptynoic acid, N-methyl-□-azido-L-lysine, and N-methyl-□-azido-D-lysine. The peptidomimetic precursor is reacted with a macrocyclization reagent such as a Ru(II) reagent on the resin as a crude mixture. For example, the reagent can be Cp*RuCl(PPh₃)₂ or [Cp*RuCl]₄ (Rasmussen et al. (2007), Org. Lett. 9:5337-5339; Zhang et al. (2005), J. Am. Chem. Soc. 127:15998-15999). In some embodiments, the macrocyclization step is performed in a solvent chosen from the group consisting of CH₂Cl₂, ClCH₂CH₂Cl, CH₃CN, DMF, and THF.

In some embodiments, a peptidomimetic macrocycle of Formula (I) comprises a halogen group substitution on a triazole moiety, for example an iodo substitution. Such peptidomimetic macrocycles may be prepared from a precursor having the partial structure and using the cross-linking methods taught herein. Crosslinkers of any length, as described herein, may be prepared comprising such substitutions. In one embodiment, the peptidomimetic macrocycle is prepared according to the scheme shown below. The reaction is performed, e.g., in the presence of CuI and an amine ligand such as TEA or TTTA. See, e.g., Hein et al. Angew. Chem., Int. Ed. 2009, 48, 8018-8021.

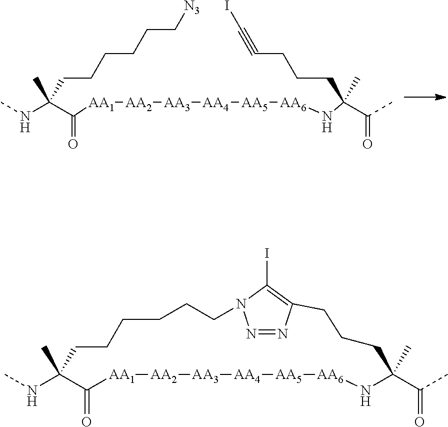

In other embodiments, an iodo-substituted triazole is generated according to the scheme shown below. For example, the second step in the reaction scheme below is performed using, e.g., CuI and N-bromosuccinimide (NBS) in the presence of THF (see, e.g., Zhang et al., J. Org. Chem. 2008, 73, 3630-3633). In other embodiments, the second step in the reaction scheme shown below is performed, e.g., using CuI and an iodinating agent such as ICl (see, e.g., Wu et al., Synthesis 2005, 1314-1318.)

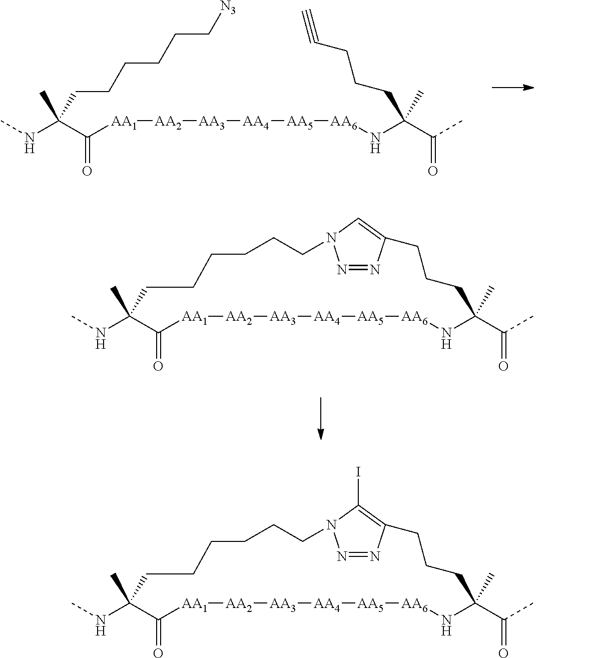

In some embodiments, an iodo-substituted triazole moiety is used in a cross-coupling reaction, such as a Suzuki or Sonogashira coupling, to afford a peptidomimetic macrocycle comprising a substituted crosslinker. Sonogashira couplings using an alkyne as shown below may be performed, e.g., in the presence of a palladium catalyst such as Pd(PPh₃)₂Cl₂, CuI, and in the presence of a base such as triethylamine. Suzuki couplings using an arylboronic or substituted alkenyl boronic acid (see below) may be performed, e.g., in the presence of a catalyst such as Pd(PPh₃)₄, and in the presence of a base such as K₂CO₃.

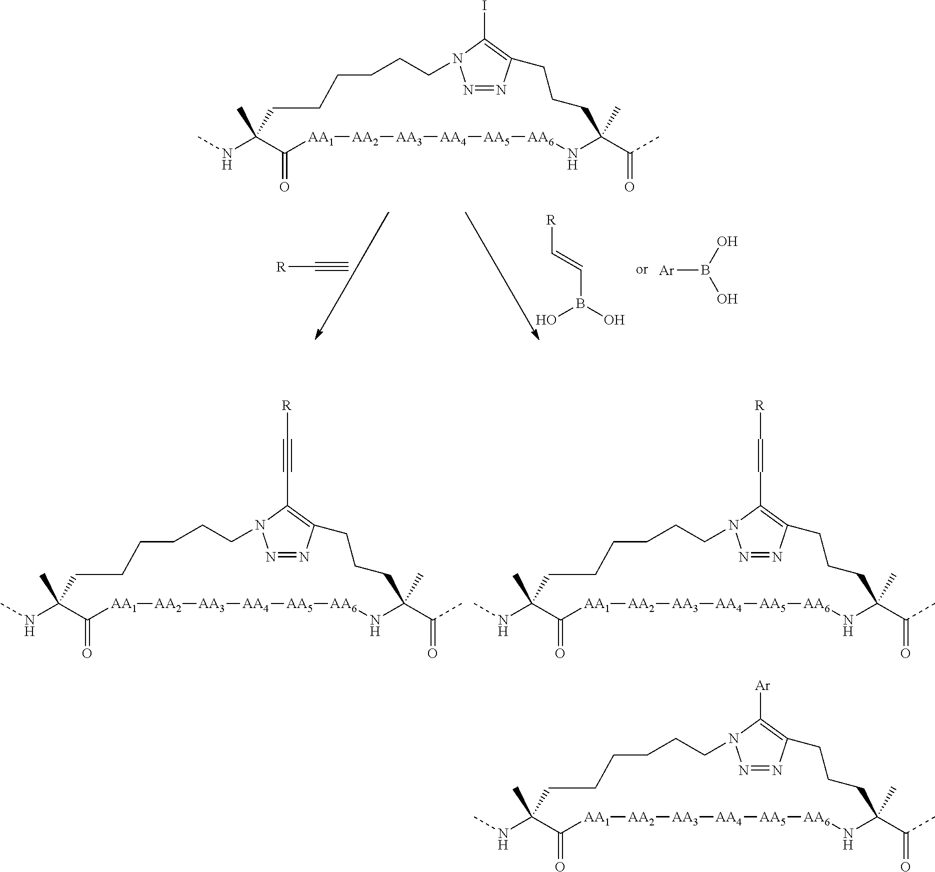

Any suitable triazole substituent groups which react with the iodo-substituted triazole can be used in Suzuki couplings described herein. Exemplary triazole substituents for use in Suzuki couplings are shown below:

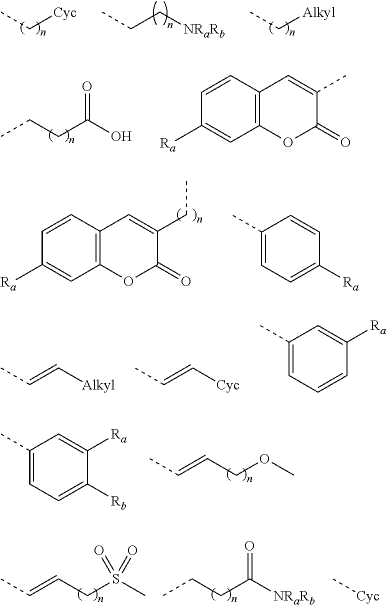

wherein “Cyc” is a suitable aryl, cycloalkyl, cycloalkenyl, heteroaryl, or heterocyclyl group, unsubstituted or optionally substituted with a Rₐ or R_{b} group as described below.

In some embodiments, the substituent is:

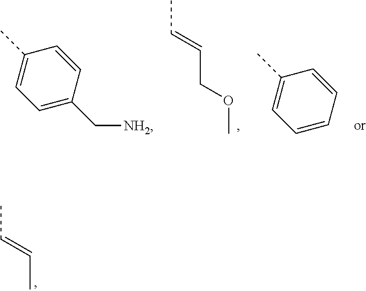

Any suitable substituent group which reacts with the iodo-substituted triazole can be used in Sonogashira couplings described herein. Example triazole substituents for use in Sonogashira couplings are shown below:

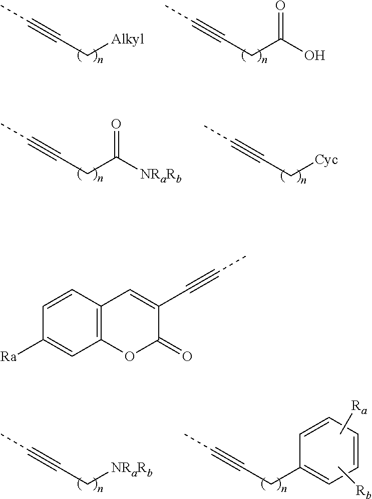

wherein “Cyc” is a suitable aryl, cycloalkyl, cycloalkenyl, heteroaryl, or heterocyclyl group, unsubstituted or optionally substituted with a Rₐ or R_{b} group as described below.

In some embodiments, the triazole substituent is:

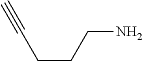

In some embodiments, the Cyc group shown above is substituted by at least one Rₐ or R_{b} substituent. In some embodiments, at least one of Rₐ and R_{b} is independently:

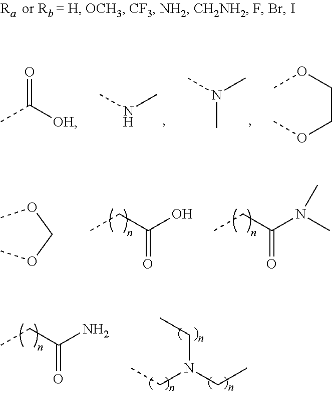

In other embodiments, the triazole substituent is

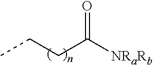

and at least one of Rₐ and R_{b} is alkyl (including —H, methyl, or ethyl), or:

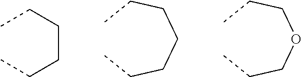

Also disclosed is use of non-naturally-occurring amino acids and amino acid analogs in the synthesis of the peptidomimetic macrocycles described herein. Any amino acid or amino acid analog amenable to the synthetic methods employed for the synthesis of stable triazole containing peptidomimetic macrocycles can be used in the present invention. For example, L-propargylglycine is contemplated as a useful amino acid in the present invention. However, other alkyne-containing amino acids that contain a different amino acid side chain are also useful in the invention. For example, L-propargylglycine contains one methylene unit between the α-carbon of the amino acid and the alkyne of the amino acid side chain. The invention also contemplates the use of amino acids with multiple methylene units between the α-carbon and the alkyne. Also, the azido-analogs of amino acids L-lysine, D-lysine, alpha-methyl-L-lysine, and alpha-methyl-D-lysine are contemplated as useful amino acids in the present invention. However, other terminal azide amino acids that contain a different amino acid side chain are also useful in the invention. For example, the azido-analog of L-lysine contains four methylene units between the α-carbon of the amino acid and the terminal azide of the amino acid side chain. The invention also contemplates the use of amino acids with fewer than or greater than four methylene units between the α-carbon and the terminal azide. Table 9 shows some amino acids useful in the preparation of peptidomimetic macrocycles disclosed herein.

TABLE 9

| | |
|---|---|
| 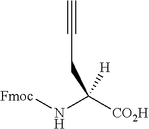 N-α-Fmoc-L- propargyl glycine | 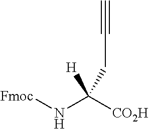 N-α-Fmoc-D- propargyl glycine |
| 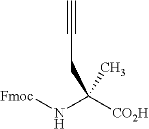 N-α-Fmoc-(S)-2-amino-2- methyl-4-pentynoic acid | 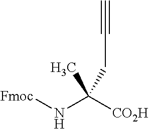 N-α-Fmoc-(R)-2-amino-2- methyl-4-pentynoic acid |
| 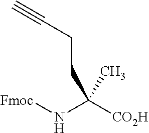 N-α-Fmoc-(S)-2-amino-2- methyl-5-hexynoic acid | 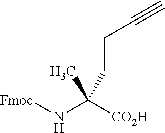 N-α-Fmoc-(R)-2-amino-2- methyl-5-hexynoic acid |
| 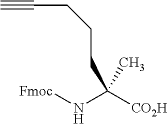 N-α-Fmoc-(S)-2-amino-2- methyl-6-heptynoic acid | 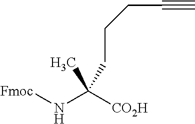 N-α-Fmoc-(R)-2-amino-2- methyl-6-heptynoic acid |
| 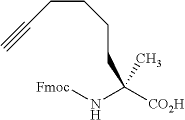 N-α-Fmoc-(S)-2-amino-2- methyl-7-octynoic acid | 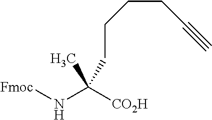 N-α-Fmoc-(R)-2-amino-2- methyl-7-octynoic acid |
| 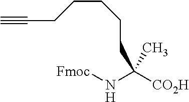 N-α-Fmoc-(S)-2-amino-2- methyl-8-nonynoic acid | 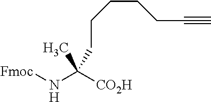 N-α-Fmoc-(R)-2-amino-2- methyl-8-nonynoic acid |
| 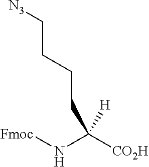 N-α-Fmoc-ε-azido- L-lysine | 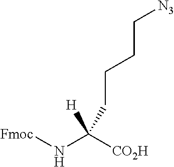 N-α-Fmoc-ε-azido- D-lysine |
| 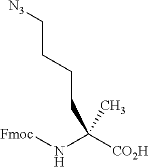 N-α-Fmoc-ε-azido- α-methyl-L-lysine | 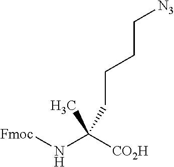 N-α-Fmoc-ε-azido- α-methyl-D-lysine |
| 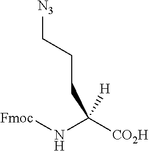 N-α-Fmoc-δ-azido- L-ornithine | 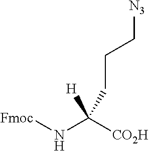 N-α-Fmoc-δ-azido- D-ornithine |
| 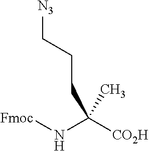 N-α-Fmoc-ε-azido- α-methyl-L- ornithine | 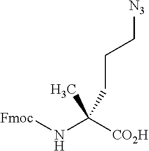 N-α-Fmoc-ε-azido- α-methyl-D- ornithine |

Table 9 shows exemplary amino acids useful in the preparation of peptidomimetic macrocycles disclosed herein.

In some embodiments the amino acids and amino acid analogs are of the D-configuration. In other embodiments they are of the L-configuration. In some embodiments, some of the amino acids and amino acid analogs contained in the peptidomimetic are of the D-configuration while some of the amino acids and amino acid analogs are of the L-configuration. In some embodiments the amino acid analogs are α,α-disubstituted, such as α-methyl-L-propargylglycine, α-methyl-D-propargylglycine, □-azido-alpha-methyl-L-lysine, and □-azido-alpha-methyl-D-lysine. In some embodiments the amino acid analogs are N-alkylated, e.g., N-methyl-L-propargylglycine, N-methyl-D-propargylglycine, N-methyl-□-azido-L-lysine, and N-methyl-□-azido-D-lysine.

In some embodiments, the —NH moiety of the amino acid is protected using a protecting group, including without limitation -Fmoc and -Boc. In other embodiments, the amino acid is not protected prior to synthesis of the peptidomimetic macrocycle.

Additional methods of forming peptidomimetic macrocycles which are envisioned as suitable to perform the present invention include those disclosed by Mustapa, M. Firouz Mohd et al., J. Org. Chem (2003), 68, pp. 8193-8198; Yang, Bin et al. Bioorg Med. Chem. Lett. (2004), 14, pp. 1403-1406; U.S. Pat. No. 5,364,851; U.S. Pat. No. 5,446,128; U.S. Pat. No. 5,824,483; U.S. Pat. No. 6,713,280; and U.S. Pat. No. 7,202,332. In such embodiments, amino acid precursors are used containing an additional substituent R— at the alpha position. Such amino acids are incorporated into the macrocycle precursor at the desired positions, which may be at the positions where the crosslinker is substituted or, alternatively, elsewhere in the sequence of the macrocycle precursor. Cyclization of the precursor is then performed according to the indicated method.

For example, a peptidomimetic macrocycle of Formula (II) is prepared as indicated:

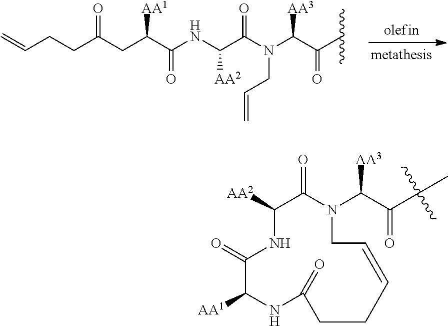

wherein each amino acid₁, amino acid₂, amino acid₃ is independently an amino acid side chain.

In other embodiments, a peptidomimetic macrocycle of Formula (II) is prepared as indicated:

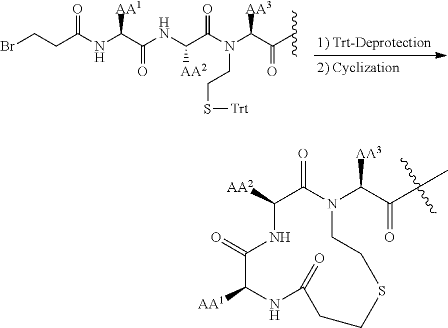

wherein each amino acid₁, amino acid₂, amino acid₃ is independently an amino acid side chain.

In some embodiments, a peptidomimetic macrocycle is obtained in more than one isomer, for example due to the configuration of a double bond within the structure of the crosslinker (E vs Z). Such isomers can or cannot be separable by conventional chromatographic methods. In some embodiments, one isomer has improved biological properties relative to the other isomer. In one embodiment, an E crosslinker olefin isomer of a peptidomimetic macrocycle has better solubility, better target affinity, better in vivo or in vitro efficacy, higher helicity, or improved cell permeability relative to its Z counterpart. In another embodiment, a Z crosslinker olefin isomer of a peptidomimetic macrocycle has better solubility, better target affinity, better in vivo or in vitro efficacy, higher helicity, or improved cell permeability relative to its E counterpart.

### Assays

The properties of the peptidomimetic macrocycles are assayed, e.g., by using the methods described below. In some embodiments, a peptidomimetic macrocycle has improved biological properties relative to a corresponding polypeptide lacking the substituents described herein.

### Assay to Determine α-Helicity

In solution, the secondary structure of polypeptides with α-helical domains reach a dynamic equilibrium between random coil structures and α-helical structures, often expressed as a “percent helicity”. Thus, e.g., α-helical domains are predominantly random coils in solution, with α-helical content usually under 25%. Peptidomimetic macrocycles with optimized linkers, on the other hand, possess, e.g., an α-helicity that is at least two-fold greater than that of a corresponding uncrosslinked polypeptide. In some embodiments, macrocycles will possess an alpha-helicity of greater than 50%. To assay the helicity of peptidomimetic macrocycles of the invention, the compounds are dissolved in an aqueous solution (e.g., 50 mM potassium phosphate solution at pH 7, or distilled H₂O, to concentrations of 25-50 M). Circular dichroism (CD) spectra are obtained on a spectropolarimeter (e.g., Jasco J-710) using standard measurement parameters (e.g., temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity (e.g., [Φ]222 obs) by the reported value for a model helical decapeptide (Yang et al. (1986), Methods Enzymol. 130:208)).

### Assay to Determine Melting Temperature™

A peptidomimetic macrocycle comprising a secondary structure such as an α-helix exhibits, e.g., a higher melting temperature than a corresponding uncrosslinked polypeptide. Typically, peptidomimetic macrocycles disclosed herein exhibit a melting temperature (T_{M}) of >60° C., representing a highly stable structure in aqueous solutions. To assay the effect of macrocycle formation on melting temperature, peptidomimetic macrocycles or unmodified peptides are dissolved in distilled H₂O (e.g., at a final concentration of 50 μM) and the T_{M} is determined by measuring the change in ellipticity over a temperature range (e.g., 4-95° C.) on a spectropolarimeter (e.g., Jasco J-710) using standard parameters (e.g., wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

### Protease Resistance Assay

The amide bond of the peptide backbone is susceptible to hydrolysis by proteases, thereby rendering peptidic compounds vulnerable to rapid degradation in vivo. Peptide helix formation, however, typically buries the amide backbone and therefore may shield it from proteolytic cleavage. The peptidomimetic macrocycles of the present invention may be subjected to in vitro trypsin proteolysis to assess for any change in degradation rate compared to a corresponding uncrosslinked polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 nm. Briefly, the peptidomimetic macrocycle and peptidomimetic precursor (5 mcg) are incubated with trypsin agarose (Pierce) (S/E ˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln [S] versus time (k=−1Xslope).

### Ex Vivo Stability Assay

Peptidomimetic macrocycles with optimized linkers possess, e.g., an ex vivo half-life that is at least two-fold greater than that of a corresponding uncrosslinked polypeptide, and possess an ex vivo half-life of 12 hours or more. For ex vivo serum stability studies, a variety of assays may be used. For example, a peptidomimetic macrocycle and a corresponding uncrosslinked polypeptide (2 mcg) are incubated with fresh mouse, rat and/or human serum (2 mL) at 37° C. for 0, 1, 2, 4, 8, and 24 hours. To determine the level of intact compound, the following procedure may be used: The samples are extracted by transferring 100 μl of sera to 2 ml centrifuge tubes followed by the addition of 10 μL of 50% formic acid and 500 μL acetonitrile and centrifugation at 14,000 RPM for 10 min at 4±2° C. The supernatants are then transferred to fresh 2 ml tubes and evaporated on Turbovap under N₂<10 psi, 37° C. The samples are reconstituted in 100 μL of 50:50 acetonitrile:water and submitted to LC-MS/MS analysis.

### In Vitro Binding Assays

To assess the binding and affinity of peptidomimetic macrocycles and peptidomimetic precursors to acceptor proteins, a fluorescence polarization assay (FPA) is used, for example. The FPA technique measures the molecular orientation and mobility using polarized light and fluorescent tracer. When excited with polarized light, fluorescent tracers (e.g., FITC) attached to molecules with high apparent molecular weights (e.g., FITC-labeled peptides bound to a large protein) emit higher levels of polarized fluorescence due to their slower rates of rotation as compared to fluorescent tracers attached to smaller molecules (e.g., FITC-labeled peptides that are free in solution).

For example, fluoresceinated peptidomimetic macrocycles (25 nM) are incubated with the acceptor protein (25-1000 nM) in binding buffer (140 mM NaCl, 50 mM Tris-HCl, pH 7.4) for 30 minutes at room temperature. Binding activity is measured, e.g., by fluorescence polarization on a luminescence spectrophotometer (e.g., Perkin-Elmer LS50B). K_{d} values may be determined by nonlinear regression analysis using, e.g., GraphPad Prism software (GraphPad Software, Inc., San Diego, Calif.). A peptidomimetic macrocycle shows, in some instances, similar or lower K_{d} than a corresponding uncrosslinked polypeptide.

### In Vitro Displacement Assays to Characterize Antagonists of Peptide-Protein Interactions

To assess the binding and affinity of compounds that antagonize the interaction between a peptide and an acceptor protein, a fluorescence polarization assay (FPA) utilizing a fluoresceinated peptidomimetic macrocycle derived from a peptidomimetic precursor sequence is used, for example. The FPA technique measures the molecular orientation and mobility using polarized light and fluorescent tracer. When excited with polarized light, fluorescent tracers (e.g., FITC) attached to molecules with high apparent molecular weights (e.g., FITC-labeled peptides bound to a large protein) emit higher levels of polarized fluorescence due to their slower rates of rotation as compared to fluorescent tracers attached to smaller molecules (e.g., FITC-labeled peptides that are free in solution). A compound that antagonizes the interaction between the fluoresceinated peptidomimetic macrocycle and an acceptor protein will be detected in a competitive binding FPA experiment.

For example, putative antagonist compounds (1 nM to 1 mM) and a fluoresceinated peptidomimetic macrocycle (25 nM) are incubated with the acceptor protein (50 nM) in binding buffer (140 mM NaCl, 50 mM Tris-HCL, pH 7.4) for 30 minutes at room temperature. Antagonist binding activity is measured, e.g., by fluorescence polarization on a luminescence spectrophotometer (e.g., Perkin-Elmer LS50B). K_{d} values may be determined by nonlinear regression analysis using, e.g., GraphPad Prism software (GraphPad Software, Inc., San Diego, Calif.).

Any class of molecule, such as small organic molecules, peptides, oligonucleotides or proteins can be examined as putative antagonists in this assay.

### Assay for Protein-Ligand Binding by Affinity Selection-Mass Spectrometry

To assess the binding and affinity of test compounds for proteins, an affinity-selection mass spectrometry assay is used, for example. Protein-ligand binding experiments are conducted according to the following representative procedure outlined for a system-wide control experiment using 1 μM peptidomimetic macrocycle plus 5 μM target protein. A 1 μL DMSO aliquot of a 40 μM stock solution of peptidomimetic macrocycle is dissolved in 19 μL of PBS (Phosphate-buffered saline: 50 mM, pH 7.5 Phosphate buffer containing 150 mM NaCl). The resulting solution is mixed by repeated pipetting and clarified by centrifugation at 10,000 g for 10 min. To a 4 μL aliquot of the resulting supernatant is added 4 μL of 10 μM target protein in PBS. Each 8.0 μL experimental sample thus contains 40 pmol (1.5 μg) of protein at 5.0 μM concentration in PBS and 1 μM peptidomimetic macrocycle and 2.5% DMSO. Duplicate samples thus prepared for each concentration point are incubated for 60 min at room temperature, and then chilled to 4° C. prior to size-exclusion chromatography-LC-MS analysis of 5.0 μL injections. Samples containing a target protein, protein-ligand complexes, and unbound compounds are injected onto an SEC column, where the complexes are separated from non-binding component by a rapid SEC step. The SEC column eluate is monitored using UV detectors to confirm that the early-eluting protein fraction, which elutes in the void volume of the SEC column, is well resolved from unbound components that are retained on the column. After the peak containing the protein and protein-ligand complexes elutes from the primary UV detector, it enters a sample loop where it is excised from the flow stream of the SEC stage and transferred directly to the LC-MS via a valving mechanism. The (M+3H)³⁺ ion of the peptidomimetic macrocycle is observed by ESI-MS at the expected m/z, confirming the detection of the protein-ligand complex.

### Assay for Protein-Ligand K_{d} Titration Experiments

To assess the binding and affinity of test compounds for proteins, a protein-ligand K_{d} titration experiment is performed. Protein-ligand K_{d} titrations experiments are conducted as follows: 2 μL DMSO aliquots of a serially diluted stock solution of titrant peptidomimetic macrocycle (5, 2.5, . . . , 0.098 mM) are prepared then dissolved in 38 μL of PBS. The resulting solutions are mixed by repeated pipetting and clarified by centrifugation at 10,000 g for 10 min. To 4.0 μL aliquots of the resulting supernatants is added 4.0 μL of 10 μM target protein in PBS. Each 8.0 μL experimental sample thus contains 40 pmol (1.5 μg) of protein at 5.0 μM concentration in PBS, varying concentrations (125, 62.5, . . . , 0.24 μM) of the titrant peptide, and 2.5% DMSO. Duplicate samples thus prepared for each concentration point are incubated at room temperature for 30 min, then chilled to 4° C. prior to SEC-LC-MS analysis of 2.0 μL injections. The (M+H)¹⁺, (M+2H)²⁺, (M+3H)³⁺, and/or (M+Na)¹⁺ ion is observed by ESI-MS; extracted ion chromatograms are quantified, then fit to equations to derive the binding affinity K_{d} as described in “A General Technique to Rank Protein-Ligand Binding Affinities and Determine Allosteric vs. Direct Binding Site Competition in Compound Mixtures.” Annis, D. A. et al., Am. Chem. Soc. (2004), 126, 15495-15503; also in D. A. Annis et al., in “Mass Spectrometry in Medicinal Chemistry,” edited by Wanner K, Hifner G: Wiley-VCH, (2007):121-184, Mannhold R, Kubinyi H, Folkers G (Series Editors): Methods and Principles in Medicinal Chemistry.

### Assay for Competitive Binding Experiments by Affinity Selection-Mass Spectrometry

To determine the ability of test compounds to bind competitively to proteins, an affinity selection mass spectrometry assay is performed, for example. A mixture of ligands at 40 M per component is prepared by combining 2 μL aliquots of 400 M stocks of each of the three compounds with 14 μL of DMSO. Then, 1 μL aliquots of this 40 μM per component mixture are combined with 1 μL DMSO aliquots of a serially diluted stock solution of titrant peptidomimetic macrocycle (10, 5, 2.5, . . . , 0.078 mM). These 2 μL samples are dissolved in 38 μL of PBS. The resulting solutions were mixed by repeated pipetting and clarified by centrifugation at 10,000 g for 10 min. To 4.0 μL aliquots of the resulting supernatants is added 4.0 μL of 10 μM target protein in PBS. Each 8.0 μL experimental sample thus contains 40 pmol (1.5 μg) of protein at 5.0 μM concentration in PBS plus 0.5 μM ligand, 2.5% DMSO, and varying concentrations (125, 62.5, . . . , 0.98 μM) of the titrant peptidomimetic macrocycle. Duplicate samples thus prepared for each concentration point are incubated at room temperature for 60 min, then chilled to 4° C. prior to SEC-LC-MS analysis of 2.0 μL injections. Additional details on these and other methods are provided in Annis et al., J. Am. Chem. Soc. (2004), 126, 15495-15503; also in Annis et al., in “Mass Spectrometry in Medicinal Chemistry,” edited by Wanner K, Höfner G: Wiley-VCH; (2007):121-184. Mannhold R, Kubinyi H, Folkers G (Series Editors): Methods and Principles in Medicinal Chemistry.

### Binding Assays in Intact Cells

It is possible to measure binding of peptides or peptidomimetic macrocycles to their natural acceptors in intact cells by immunoprecipitation experiments. For example, intact cells are incubated with fluoresceinated (FITC-labeled) compounds for 4 hrs in the absence of serum, followed by serum replacement and further incubation that ranges from 4-18 hrs. Cells are then pelleted and incubated in lysis buffer (50 mM Tris [pH 7.6], 150 mM NaCl, 1% CHAPS and protease inhibitor cocktail) for 10 minutes at 4° C. Extracts are centrifuged at 14,000 rpm for 15 minutes and supernatants collected and incubated with 10 μl goat anti-FITC antibody for 2 hrs, rotating at 4° C. followed by 2 hrs incubation at 4° C. with protein A/G Sepharose (50 μl of 50% bead slurry). After quick centrifugation, the pellets are washed in lysis buffer containing increasing salt concentration (e.g., 150, 300, 500 mM). The beads are then re-equilibrated at 150 mM NaCl before addition of SDS-containing sample buffer and boiling. After centrifugation, the supernatants are optionally electrophoresed using 4%-12% gradient Bis-Tris gels followed by transfer into Immobilon-P membranes. After blocking, blots are optionally incubated with an antibody that detects FITC and also with one or more antibodies that detect proteins that bind to the peptidomimetic macrocycle.

### Cellular Penetrability Assays

To measure the cell penetrability of peptidomimetic macrocycles and corresponding uncrosslinked macrocycle, intact cells are incubated with fluoresceinated peptidomimetic macrocycles or corresponding uncrosslinked macrocycle (10 βM) for 4 hrs in serum free media at 37° C., washed twice with media and incubated with trypsin (0.25%) for 10 min at 37° C. The cells are washed again and resuspended in PBS. Cellular fluorescence is analyzed, e.g., by using either a FACSCalibur flow cytometer or Cellomics' KineticScan® HCS Reader.

### In Vivo Stability Assay

To investigate the in vivo stability of the peptidomimetic macrocycles, the compounds are, for example, administered to mice and/or rats by IV, IP, PO or inhalation routes at concentrations ranging from 0.1-50 mg/kg and blood specimens withdrawn at 0′, 5′, 15′, 30′, 1 hr, 4 hrs, 8 hrs and 24 hours post-injection. Levels of intact compound in 25 L of fresh serum are then measured by LC-MS/MS as above.

### Clinical Trials

To determine the suitability of the peptidomimetic macrocycles provided herein for treatment of humans, clinical trials are performed. For example, patients diagnosed with a PTH-related disorder, for example hyperparathyroidism, hypercalcemia, or hypoparathyroidism and in need of treatment are selected and separated in treatment and one or more control groups, wherein the treatment group is administered a peptidomimetic macrocycle of the invention, while the control groups receive a placebo or a known PTH drug. The treatment safety and efficacy of the peptidomimetic macrocycles provided herein can thus be evaluated by performing comparisons of the patient groups with respect to factors such as survival and quality-of-life. In this example, the patient group treated with a peptidomimetic macrocycle show improved long-term survival compared to a patient control group treated with a placebo.

### Pharmaceutical Compositions and Routes of Administration

A pharmaceutical composition is provided comprising a peptidomimetic macrocycle provided herein and a pharmaceutically acceptable carrier.

The peptidomimetic macrocycles provided herein also include pharmaceutically acceptable derivatives or prodrugs thereof. A “pharmaceutically acceptable derivative” means any pharmaceutically acceptable salt, ester, salt of an ester, pro-drug or other derivative of a compound of this invention which, upon administration to a recipient, is capable of providing (directly or indirectly) a compound of this invention. Particularly favored pharmaceutically acceptable derivatives are those that increase the bioavailability of the compounds when administered to a mammal (e.g., by increasing absorption into the blood of an orally administered compound) or which increases delivery of the active compound to a biological compartment (e.g., the brain or lymphatic system) relative to the parent species. Some pharmaceutically acceptable derivatives include a chemical group which increases aqueous solubility or active transport across the gastrointestinal mucosa.

In some embodiments, the peptidomimetic macrocycles are modified by covalently or non-covalently joining appropriate functional groups to enhance selective biological properties. Such modifications include those which increase biological penetration into a given biological compartment (e.g., blood, lymphatic system, central nervous system), increase oral availability, increase solubility to allow administration by injection, alter metabolism, and alter rate of excretion.

Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts.

For preparing pharmaceutical compositions from the compounds of the present invention, pharmaceutically acceptable carriers include either solid or liquid carriers. Solid form preparations include powders, tablets, pills, capsules, cachets, suppositories, and dispersible granules. A solid carrier can be one or more substances, which also acts as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa.

In powders, the carrier is a finely divided solid, which is in a mixture with the finely divided active component. In tablets, the active component is mixed with the carrier having the necessary binding properties in suitable proportions and compacted in the shape and size desired.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents are added, such as the cross-linked polyvinyl pyrrolidone, agar, alginic acid, or a salt thereof, such as sodium alginate.

Liquid form preparations include solutions, suspensions, and emulsions, e.g., water or water/propylene glycol solutions. For parenteral injection, liquid preparations can be formulated in solution in aqueous polyethylene glycol solution.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

When the compositions of this invention comprise a combination of a peptidomimetic macrocycle and one or more additional therapeutic or prophylactic agents, both the compound and the additional agent should be present at dosage levels of between about 1-100%, and more preferably between about 5-95% of the dosage normally administered in a monotherapy regimen. In some embodiments, the additional agents are administered separately, as part of a multiple dose regimen, from the compounds of this invention. Alternatively, those agents are part of a single dosage form, mixed together with the compounds of this invention in a single composition.

In some embodiments, the compositions are present as unit dosage forms that can deliver, e.g., from about 0.0001-1,000 mg of the peptidomimetic macrocycles, salts thereof, prodrugs thereof, derivatives thereof, or any combination of these. Thus, the unit dosage forms can deliver, e.g., in some embodiments, from about 1-900 mg, from about 1-800 mg, from about 1-700 mg, from about 1-600 mg, from about 1-500 mg, from about 1-400 mg, from about 1-300 mg, from about 1-200 mg, from about 1-100 mg, from about 1-10 mg, from about 1-5 mg, from about 0.1-10 mg, from about 0.1-5 mg, from about 10-1,000 mg, from about 50-1,000 mg, from about 100-1,000 mg, from about 200-1,000 mg, from about 300-1,000 mg, from about 400-1,000 mg, from about 500-1,000 mg, from about 600-1,000 mg, from about 700-1,000 mg, from about 800-1,000 mg, from about 900-1,000 mg, from about 10-900 mg, from about 100-800 mg, from about 200-700 mg, or from about 300-600 mg of the peptidomimetic macrocycles, salts thereof, prodrugs thereof, derivatives thereof, or any combination of these.

In some embodiments, the compositions are present as unit dosage forms that can deliver, e.g., about 1 mg, about 2 mg, about 3 mg, about 4 mg, about 5 mg, about 6 mg, about 7 mg, about 8 mg, about 9 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 500 mg, about 600 mg, about 700 mg, about 800 mg, or about 800 mg of peptidomimetic macrocycles, salts thereof, prodrugs thereof, derivatives thereof, or any combination of these.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a composition as described herein is administered in a local rather than systemic manner, e.g., via injection of the compound directly into an organ. In specific embodiments, long acting formulations are administered by implantation (for example subcutaneously or intramuscularly) or by intramuscular injection. In other embodiments, the drug is delivered in a targeted drug delivery system, e.g., in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In another embodiment, compositions described herein are formulated for oral administration. Compositions described herein are formulated by combining a peptidomimetic macrocycle with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the peptidomimetic macrocycles described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: e.g., maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In certain embodiments, dosage forms, such as dragee cores and tablets, are provided with one or more suitable coating. In specific embodiments, concentrated sugar solutions are used for coating the dosage form. The sugar solutions optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the peptidomimetic macrocycles described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the peptidomimetic macrocycles described herein are formulated for buccal or sublingual administration. Formulations suitable for buccal or sublingual administration include, by way of example only, tablets, lozenges, or gels. In still other embodiments, the peptidomimetic macrocycles described herein are formulated for parenteral injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, pharmaceutical compositions are formulated in a form suitable for parenteral injection as a sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, the active ingredient is in powder form for constitution with a suitable vehicle, e.g., sterile pyrogen-free water, before use.

Pharmaceutical compositions herein can be administered, e.g., once or twice or three or four or five or six times per day, or once or twice or three or four or five or six times per week, and can be administered, e.g., for a day, a week, a month, 3 months, six months, a year, five years, or for example ten years. In some embodiments, a pharmaceutical formulation is administered no more frequently than once daily, no more frequently than every other day, no more frequently than twice weekly, no more frequently than three times weekly, no more frequently than four times weekly, no more frequently than five times weekly, or no more frequently than every other week. In some embodiments, a pharmaceutical formulation is administered no more than once weekly. In some embodiments, a pharmaceutical formulation is administered no more than twice weekly. In some embodiments, a pharmaceutical formulation is administered no more than three times weekly. In some embodiments, a pharmaceutical formulation is administered no more than four times weekly. In some embodiments, a pharmaceutical formulation is administered no more than five times weekly.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope and that methods and structures within the scope of these claims and their equivalents be covered thereby.

### Methods of Use

As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The parathyroid glands produce PTH that regulates the calcium level in the blood. PTH, when chronically produced in excess (hyperparathyroidism), takes calcium out of bone and brings it into the blood. When this hormone is given by daily injection that lasts only a few hours each day, it has the opposite effect on bone and builds bone.

Calcium plays an indispensable role in cell permeability, the formation of bones and teeth, blood coagulation, transmission of nerve impulse, and normal muscle contraction. The concentration of calcium ions in the blood is, along with calcitrol and calcitonin, regulated mainly by parathyroid hormone (PTH). Although calcium intake and excretion may vary, PTH serves through a feedback mechanism to maintain a steady concentration of calcium in cells and surrounding fluids. When serum calcium lowers, the parathyroid glands secrete PTH, affecting the release of stored calcium. When serum calcium increases, stored calcium release is retarded through lowered secretions of PTH.

A method is disclosed for treating a condition characterized by increased or decreased activity or production of PTH or PTHrP in a subject in need thereof, comprising administering to the subject an effective amount of a composition comprising a peptidomimetic macrocycle as disclosed herein. A method is disclosed for treating a condition characterized by increased or decreased activity or production of PTH or PTHrP in a subject in need thereof, comprising administering to the subject an effective amount of a composition comprising a peptidomimetic macrocycle as disclosed herein.

In some embodiments, the condition is hypoparathyroidism. In some embodiments, the condition is hyperparathyroidism or hypercalcemia. In some embodiments, the condition is primary hyperparathyroidism. In some embodiments, the subject suffers from a parathyroid adenoma, parathyroid hyperplasia, or a parathyroid carcinoma. In some embodiments, the parathyroid carcinoma is inoperable parathyroid tumor. In some embodiments, the inoperable parathyroid tumor is metaphyseal chondrodysplasia. In some embodiments, the subject suffers from a multiple endocrine neoplasia or familial hyperparathyroidism. In some embodiments, the condition is secondary hyperparathyroidism. In some embodiments, the subject suffers from a renal disorder or vitamin D deficiency. In some embodiments, the renal disorder is chronic kidney disease. In some embodiments, the chronic kidney disease is in stage 1, 2, 3 or 4. In some embodiments, the subject is undergoing dialysis. In some embodiments, the condition is tertiary hyperparathyroidism.

A method is disclosed for decreasing the activity of PTH or PTHrP in a subject in need thereof, comprising administering to the subject an effective amount of a composition comprising a peptidomimetic macrocycle as disclosed herein. Also disclosed is a method for increasing the activity of PTH or PTHrP in a subject in need thereof, comprising administering to the subject an effective amount of a composition comprising a peptidomimetic macrocycle as disclosed herein. A method is disclosed for treating a condition of skin or hair, comprising administering to the subject an effective amount of a composition comprising a peptidomimetic macrocycle as disclosed herein. A method is disclosed for treating a condition of skin or hair, comprising administering to the subject an effective amount of a composition comprising a peptidomimetic macrocycle as disclosed herein. In some embodiments, the disorder is insufficient hair growth. In some embodiments, the disorder is psoriasis.

A method is disclosed for treating a condition characterized by a decrease in bone mass or insufficient bone mass in a subject, comprising administering to the subject an effective amount of a composition comprising a peptidomimetic macrocycle as disclosed herein. A method is disclosed for treating a condition characterized by an increase in bone mass or insufficient bone mass in a subject, comprising administering to the subject an effective amount of a composition comprising a peptidomimetic macrocycle as disclosed herein. In some embodiments, the condition is osteoporosis. In some embodiments, the condition is osteopenia.

In some embodiments, a peptidomimetic macrocycle is administered parenterally. In some embodiments, a peptidomimetic macrocycle is administered subcutaneously. In some embodiments, a peptidomimetic macrocycle is administered intravenously. In some embodiments, administering is no more frequently than once daily, no more frequently than every other day, no more frequently than three times weekly, no more frequently than twice weekly, no more frequently than weekly, or no more frequently than every other week. In some embodiments, administering is no more frequently than three times weekly. In some embodiments, administering is no more frequently than weekly, for example once weekly.

In one aspect, peptidomimetic macrocycles are provided that are useful in competitive binding assays to identify agents which bind to the natural ligand(s) of the proteins or peptides upon which the peptidomimetic macrocycles are modeled. For example, in the PTH system, labeled peptidomimetic macrocycles based on PTH and/or PTHrP can be used in a binding assay along with small molecules that competitively bind to the PTH receptor. Competitive binding studies allow for rapid in vitro evaluation and determination of drug candidates specific for the PTH system. Such binding studies can be performed with the peptidomimetic macrocycles disclosed herein and their binding partners.

The invention further provides for the generation of antibodies against the peptidomimetic macrocycles. In some embodiments, these antibodies specifically bind both the peptidomimetic macrocycle and the precursor peptides, such as PTH, to which the peptidomimetic macrocycles are related. Such antibodies, e.g., disrupt the native protein-protein interactions, e.g., between PTH and the PTH receptor. The PTH receptor or PTHrP receptor may be a PTH/PTHrP type I or type II receptor.

In other aspects, the disclosure provides for both prophylactic and therapeutic methods of treating a subject at risk of (or susceptible to) a disorder or having a disorder associated with aberrant (e.g., insufficient or excessive) expression or activity of the molecules including PTH-family proteins, such as PTH and PTHrP.

In another embodiment, a disorder is caused, at least in part, by an abnormal level of PTH, (e.g., over or under expression), or by the presence of PTH exhibiting abnormal activity. As such, the reduction in the level and/or activity of PTH or the enhancement of the level and/or activity of PTH, by peptidomimetic macrocycles derived from PTH, is used, e.g., to ameliorate or reduce the adverse symptoms of the disorder.

In another aspect, the present invention provides methods for treating or preventing a disease including hyperparathyroidism and hypoparathyroidism by interfering with the interaction or binding between binding partners, e.g., between PTH and PTH receptor. These methods comprise administering an effective amount of a compound to a warm blooded animal, including a human. Hyperparathyroidism can be triggered by parathyroid adenoma, hereditary factors, parathyroid carcinoma, or renal osteodystrophy.

In some embodiments, a peptidomimetic macrocycle is used to treat, prevent, and/or diagnose parathyroidisms. Examples of parathyroidisms include, but are not limited to, hyperparathyroidism, primary hyperparathyroidism, primary hyperparathyroidism associated with multiple endocrine neoplasia (MEN), secondary hyperparathyroidism, tertiary hyperparathyroidism, hypoparathyroidism, familial hyperparathyroidism, pseudohypoparathyroidism, pseudopseudohypoparathyroidism, parathyroid disease, diseases of the parathyroid gland, kidney stones, renal failure, vitamin D deficiency, and parathyroiditis. Primary hyperparathyroidism is a hormonal problem that occurs when one or more of the parathyroid glands produce too much PTH. The blood calcium becomes higher than normal, bones may lose calcium and kidney stones may form. Hyperparathyroidism can lead to loss of appetite, nausea, vomiting, constipation, confusion or impaired thinking and memory, and increased thirst and urination. Primary hyperparathyroidism associated with multiple endocrine neoplasia (MEN), is a condition in which primary hyperparathyroidism is associated with tumors in other endocrine organs such as the pituitary and pancreas. MEN is a familial condition which involves genetic and hormonal abnormalities. Secondary hyperparathyroidism is a condition in which the parathyroid hormone is elevated in response to kidney failure or to inadequate calcium or vitamin D (e.g., caused by vitamin D deficiency, intestinal or stomach surgery, or intestinal disease). In the absence of kidney failure, secondary hyperparathyroidism is often caused by vitamin D deficiency or stomach or intestinal disorders. Hypoparathyroidism is a condition in which the parathyroid glands have been removed surgically or do not function for other reasons. This causes low blood calcium. In some embodiments, the peptidomimetic macrocycles provided herein is used to treat, prevent, and/or diagnose a patient being treated with dialysis. In some embodiments, the peptidomimetic macrocycles provided herein is used to treat, prevent, and/or diagnose a patient not being treated with dialysis. In some embodiments, a patient being treated with dialysis administered a pharmaceutical formulation provided herein no more than three times weekly, four times weekly, or five times weekly.

In some embodiments, a peptidomimetic macrocycle provided herein is used to treat, prevent, and/or diagnose parathyroid tumors. Examples of parathyroid tumors include, but are not limited to, parathyroid carcinoma, parathyroid adenoma, parathyroid hyperplasia, multiple endocrine neoplasia types I and II, and lymphomas and metastases.

In some embodiments, a peptidomimetic macrocycle provided herein is used to treat, prevent, and/or diagnose disorders of the parathyroid hormone receptor. Examples of parathyroid carcinomas include, but are not limited to, Jansen metaphyseal chondrodysplasia, Jansen disease, Jansen metaphyseal dysostosis, Murk Jansen type metaphyseal chondrodysplasia, or Blomstrand's chondroplasia. See, e.g., Jansen SE. “Metaphyseal Chondrodysplasia” in: “NORD Guide to Rare Disorders,” Philadelphia, Pa.: Lippincott Williams & Wilkins; 2003:559.

In some embodiments, a peptidomimetic macrocycle provided herein is used to treat, prevent, and/or diagnose skeletal disorders. Examples of skeletal disorders include, but are not limited to, osteoporosis, osteopenia, osteopetrosis, osteomalacia, osteitis fibrosa cystic, osteitis fibrosa, osteodystrophia fibrosa, Von Recklinghausen's Disease of Bone, Paget's disease of bone, renal osteodystrophy, fibrous dysplasia bone, McCune-Albright syndrome, osteogenesis imperfect, hypophosphatasia, disorders of phosphate metabolism, disorders of abnormally high bone density/osteosclerosis, extraskeletal calcification/ossification, adynamic bone disease, gangrene, bone pain, bone fractures, muscle weakness, diffuse calcification in the skin, soft tissues, and arteries (calciphylaxis), ischemic necrosis of the skin, gangrene, cardiac arrhythmias, pulmonary failure, and rickets.

In some embodiments, a peptidomimetic macrocycle provided herein is used to treat a disorder of the skin or hair. In some embodiments, a peptidomimetic macrocycle is used to treat psoriasis, enhance epidermal growth of aged skin, enhance wound healing, or stimulate hair growth in an animal, for example in a human subject. See, e.g., Holick et al. Proc. Natl. Acad. Sci. 91:8014-8016.

In some embodiments, a peptidomimetic macrocycle provided herein is used to treat, prevent, and/or diagnose syndromes associated with malignancy. Examples of syndromes associated with malignancy include, but are not limited to, digestive system disorders, such as diarrhea, vomiturition and nausea; proteometabolism abnormality, such as hypoalbuminemia; saccharometabolism abnormality, such as reduction of glucose tolerance and reduction of insulin secretion; lipid metabolism abnormality, such as hyperlipidemia and reduction of serum lipoprotein lipase activity; anorexia; hematological abnormality, such as hyperlipidemia and reduction of serum lipoprotein lipase activity; electrolyte abnormality, such as hyponatremia, hypokalemia, hypocalciuric hypercalcemia, and hypercalcemia; immunodeficiency, such as an infectious disease; pain; secondary hyperparathyroidism; and primary hyperparathyroidism. Hypercalcemia (high blood calcium) is a disorder that most commonly results from primary hyperparathyroidism. High blood calcium levels can contribute to other problems that can be treated, prevented, and/or diagnosed with the peptidomimetic macrocycles provided herein including, but not limited to, heart disease, high blood pressure, and difficulty with concentration. Hypocalcemia (low blood calcium) is a disorder with inadequate calcium in the blood. A variety of conditions such as vitamin D deficiency, intestinal disease, and hypoparathyroidism can cause low blood calcium.

In some embodiments, a peptidomimetic macrocycle provided herein is used to treat, prevent, and/or diagnose central nervous system diseases. Examples of central nervous system diseases include, but are not limited to, dyssomnia; neuropathy, such as schizophrenia, manic-depressive psychosis, neurosis and psychophysiological disorder; nervous symptom, such as vomitation, nausea, mouth dryness, anorexia and vertigo; brain metabolism abnormality, cerebral circulation abnormality, autonomic imbalance, and endocrine system abnormality with which central nervous system is associated.

In some embodiments, a peptidomimetic macrocycle provided herein is used to treat, prevent, and/or diagnose a disease caused by PTH or PTHrP-cytokine cascade, which comprises, as an active ingredient, an agonist or antagonist binding to a PTH receptor or PTHrP receptor, or a substance binding to a ligand of the receptor to promote or inhibit binding between the ligand and the receptor. Examples of cytokines may include IL-1, IL-2, IL-3, IL-4, IL-5, IL-6, IL-7, IL-8, IL-9, IL-10, IL-11, IL-12, IL-13, IL-15, G-CSF, GM-CSF, M-CSF, EPO, LIF, TPO, EGF, TGF-α, TGF-β, FGF, IGF, HGF, VEGF, NGF, activin, inhibin, a BMP family, TNF and IFN, etc. Examples of diseases caused by PTH or PTHrP-cytokine cascade may include septicemia, cachexia, inflammation, hemopathy such as hematopoietic system abnormality and leukemia, calcium metabolism abnormality, and autoimmune disease such as rheumatism.

Another embodiment of this aspect relates to a method of treating or preventing in a subject in need thereof a disorder mediated by interaction of PTH and/or PTHrP with a PTH receptor. This method involves administering a peptide of the present invention to the subject under conditions effective to treat or prevent the disorder.

In some embodiments, a method for treating a condition characterized by increased activity or production of PTH or PTHrP in a subject in need thereof, comprises administering to the subject an effective amount of a composition comprising a peptidomimetic macrocycle described herein.

In some embodiments, the condition is hyperparathyroidism. In some embodiments, the condition is primary hyperparathyroidism. In some embodiments, the subject suffers from a parathyroid adenoma, parathyroid hyperplasia, or a parathyroid carcinoma. In some embodiments, the subject suffers from a multiple endocrine neoplasia or familial hyperparathyroidism. In some embodiments, the condition is secondary hyperparathyroidism.

In some embodiments, the subject suffers from a renal disorder or vitamin D deficiency. In some embodiments, the renal disorder is chronic kidney disease. Kidney disease is a chronic, progressive disease and specific symptoms are associated with its progression. Many symptoms are associated with what is known as the Glomerular Filtration Rate (GFR). According to the Foundation for IgA Nephropathy, the GFR is the rate at which the kidneys filter waste and relates to a patient's kidney function. “Stage 1” includes signs of mild kidney disease but normal or better GFR (greater than 90% kidney function). “Stage 2” includes signs of mild kidney disease with reduced GFR (about 60% to about 89% kidney function). “Stage 3” includes signs of moderate chronic renal insufficiency with reduced GFR (about 40% to about 59% kidney function). “Stage 4” includes signs of severe chronic renal insufficiency with reduced GFR (about 15%-29% kidney function). “Stage 5” includes signs of end stage renal failure with a GFR indicating less than 15% kidney function. In some embodiments, a subject can be selected for treatment with the peptidomimetic macrocycles based on a diagnosis by a nephrologist.

In some embodiments, a subject can be selected for treatment with a peptidomimetic macrocycle provided herein based on the expression levels of suitable biomarkers for the disease. For example, a subject can be selected for treatment with a peptidomimetic macrocycle based on the expression levels of one or more of the following biomarkers: proliferating cell nuclear antigen (PCNA), blood urea nitrogen, creatinine, phosphorus, ionized calcium, PTH, PTHrP, osteocalcin, tartrate-resistant acid phosphatase, cAMP, and vitamin D3. In some embodiments, a subject can be selected for treatment with the peptidomimetic macrocycles based on bone mineral density (BMD), bone calcium, bone architecture, or serum total calcium.

In some embodiments, the subject is undergoing dialysis.

In some embodiments, the condition is tertiary hyperparathyroidism.

In some embodiments, a method for decreasing the activity or production of PTH or PTHrP in a subject in need thereof, comprises administering to the subject an effective amount of a composition comprising a peptidomimetic macrocycle described herein.

In some embodiments, a method is disclosed for treating a condition characterized by a decrease in bone mass in a subject, comprising administering to the subject an effective amount of any composition comprising a peptidomimetic macrocycle described herein.

In some embodiments, the peptidomimetic macrocycle is administered parenterally. In some embodiments, the peptidomimetic macrocycle is administered subcutaneously. In some embodiments, the peptidomimetic macrocycle is administered intravenously.

In some embodiments, peptidomimetic macrocycles are administered in combination with one or more agents. In some embodiments, the agent is a calcimimetic. In one embodiment, the agent is AMG-073 HCl (cinacalcet HCl). In another embodiment, the agent is 3-(2-chlorophenyl)-N-((1R)-1-(3-methoxyphenyl)ethyl)-1-propanamine (R-568). In still another embodiment, the agent is AMG 416. In still another embodiment, the agent is ONO-5163 (formerly KAI-4169).

In another aspect, the present invention provides methods for treating or preventing a disease including cancer cachexia. Neutralization of PTHrP or PTH might hold promise for ameliorating cancer cachexia and improve patient survival (See, e.g., Kier et al., Nature. 513 (7516):100-4). In some embodiments, a subject has cachexia and a cancer. In some embodiments, a subject has a wasting disorder of adipose tissue. In some embodiments, a subject has a wasting disorder of skeletal muscle tissues. In some embodiments, a subject exhibits weight loss. In some embodiments, a subject exhibits frailty. In some embodiments, a subject has a higher resting energy expenditure level than in healthy individuals. In some embodiments, a subject has greater thermogenesis in brown fat than in healthy individual. In some embodiments, a subject has browning of adipose tissue.

### EXAMPLES

### Example 1

### Peptidomimetic Macrocycles of the Invention

Peptidomimetic macrocycles were synthesized, purified and analyzed as previously described and as described below (Schafmeister et al., J. Am. Chem. Soc. 122:5891-5892 (2000); Schafmeister & Verdine, J. Am. Chem. Soc. 122:5891 (2005); Walensky et al., Science 305:1466-1470 (2004); and U.S. Pat. No. 7,192,713). Peptidomimetic macrocycles were designed by replacing two or more naturally occurring amino acids with the corresponding synthetic amino acids. Substitutions were made at i and i+4, and i and i+7 positions. Peptide synthesis was performed manually or on an automated peptide synthesizer (Applied Biosystems, model 433A), using solid phase conditions, rink amide AM resin (Novabiochem), and Fmoc main-chain protecting group chemistry. For the coupling of natural Fmoc-protected amino acids (Novabiochem), 10 equivalents of amino acid and a 1:1:2 molar ratio of coupling reagents HBTU/HOBt (Novabiochem)/DIEA were employed. Non-natural amino acids (4 equiv) were coupled with a 1:1:2 molar ratio of HATU (Applied Biosystems)/HOBt/DIEA. The N-termini of the synthetic peptides were acetylated, while the C-termini were amidated.

Purification of cross-linked compounds was achieved by high performance liquid chromatography (HPLC) (Varian ProStar) on a reverse phase C18 column (Varian) to yield the pure compounds. Chemical composition of the pure products was confirmed by LC/MS mass spectrometry (Micromass LCT interfaced with Agilent 1100 HPLC system) and amino acid analysis (Applied Biosystems, model 420A) (Table 10).

TABLE 10

| | Calc'd | | | | Calc'd | | |
|---|---|---|---|---|---|---|---|
| | Exact | Calc'd | Obsv'd | | Exact | Calc'd | Obsv'd |
| SP# | Mass | [M + 3]/3 | [M + 3]/3 | SP# | Mass | [M + 3]/3 | [M + 3]/3 |
| 132 | 4120.32 | 1374.45 | 1374.95 | 150 | 3894.22 | 1299.08 | 1299.83 |
| 142 | 4123.3 | 1375.44 | 1375.87 | 151 | 3813.16 | 1272.06 | 1272.54 |
| 143 | 4123.3 | 1375.44 | 1375.87 | 152 | 3722.15 | 1241.72 | 1242.2 |
| 57 | 3477.00 | 1160.00 | 1160.61 | 153 | 3766.14 | 1256.39 | 1256.91 |
| 112 | 3479.98 | 1160.99 | 1161.54 | 154 | 3738.11 | 1247.04 | 1247.47 |
| 113 | 3479.98 | 1160.99 | 1161.54 | 155 | 3894.22 | 1299.08 | 1299.83 |
| 114 | 3813.13 | 1272.04 | 1272.82 | 156 | 3795.14 | 1266.05 | 1266.9 |
| 2 | 3442.98 | 1148.66 | 1149.24 | 157 | 3921.25 | 1308.09 | 1308.62 |
| 3 | 3434.94 | 1145.98 | 1146.55 | 158 | 3828.17 | 1277.06 | 1277.63 |
| 4 | 3457.92 | 1153.64 | 1154.23 | 159 | 3476.99 | 1160 | 1160.52 |
| 5 | 3441.96 | 1148.32 | 1148.86 | 160 | 3507.01 | 1170.01 | 1170.51 |
| 6 | 3400.93 | 1134.64 | 1135.17 | 161 | 3549.02 | 1184.01 | 1184.57 |
| 7 | 3458.92 | 1153.97 | 1154.51 | 162 | 3523.01 | 1175.34 | 1175.97 |
| 8 | 3427.95 | 1143.65 | 1144.15 | 163 | 3553.03 | 1185.35 | 1186.33 |
| 9 | 3429.92 | 1144.31 | 1144.89 | 164 | 3517.02 | 1173.35 | 1174.03 |
| 10 | 3442.96 | 1148.65 | 1149.24 | 165 | 3440.99 | 1148 | 1148.59 |
| 11 | 3498.98 | 1167.33 | 1167.83 | 166 | 3507.01 | 1170.01 | 1170.6 |
| 12 | 3442.96 | 1148.65 | 1149.14 | 167 | 3494.98 | 1166 | 1166.63 |
| 13 | 3434.94 | 1145.98 | 1146.55 | 168 | 3519.01 | 1174.01 | 1174.95 |
| 14 | 3469.96 | 1157.65 | 1158.39 | 169 | 3519.01 | 1174.01 | 1174.67 |
| 15 | 3514.02 | 1172.34 | 1172.92 | 170 | 3449.01 | 1150.68 | 1151.27 |
| 16 | 3458.92 | 1153.97 | 1154.51 | 171 | 3565.02 | 1189.35 | 1189.94 |
| 17 | 3433.98 | 1145.66 | 1146.18 | 172 | 3523.01 | 1175.34 | 1176.06 |
| 18 | 3419.93 | 1140.98 | 1141.65 | 173 | 3507.01 | 1170.01 | 1170.97 |
| 19 | 3514.98 | 1172.66 | 1173.29 | 174 | 3519.01 | 1174.01 | 1174.58 |
| 20 | 3458.92 | 1153.97 | 1154.88 | 175 | 3547.04 | 1183.35 | 1184.02 |
| 21 | 4086.30 | 1363.10 | 1363.11 | 176 | 3519.01 | 1174.01 | 1174.67 |
| 22 | 4078.26 | 1360.42 | 1361.16 | 177 | 3541 | 1181.34 | 1182.35 |
| 23 | 4086.28 | 1363.09 | 1363.75 | 178 | 3507.01 | 1170.01 | 1170.51 |
| 24 | 4128.29 | 1377.10 | 1378.00 | 179 | 3556.99 | 1186.67 | 1187.35 |
| 25 | 4101.28 | 1368.09 | 1368.47 | 180 | 3530.99 | 1178 | 1178.65 |
| 26 | 4128.29 | 1377.10 | 1377.63 | 181 | 3555.01 | 1186.01 | 1186.7 |
| 27 | 4170.33 | 1391.11 | 1391.69 | 182 | 3440.99 | 1148 | 1148.59 |
| 28 | 3484.99 | 1162.66 | 1163.20 | 183 | 3517.02 | 1173.35 | 1173.84 |
| 29 | 3433.97 | 1145.66 | 1146.27 | 184 | 3456.99 | 1153.34 | 1154.05 |
| 31 | 3514.98 | 1172.66 | 1173.29 | 185 | 3499 | 1167.34 | 1168.01 |
| 33 | 3470.03 | 1157.68 | 1158.39 | 186 | 3440.99 | 1148 | 1148.77 |
| 34 | 3514.98 | 1172.66 | 1173.29 | 187 | 3517.02 | 1173.35 | 1174.3 |
| 35 | 3469.99 | 1157.66 | 1158.12 | 188 | 3456.99 | 1153.34 | 1153.86 |
| 36 | 3457.95 | 1153.65 | 1154.32 | 189 | 3499 | 1167.34 | 1168.01 |
| 38 | 3527.02 | 1176.67 | 1177.17 | 190 | 3519.01 | 1174.01 | 1174.58 |
| 40 | 3485.01 | 1162.67 | 1163.20 | 191 | 3519.01 | 1174.01 | 1174.77 |
| 42 | 3491.01 | 1164.67 | 1165.24 | 192 | 3546.02 | 1183.01 | 1183.83 |
| 44 | 3541.07 | 1181.36 | 1181.89 | 193 | 3557.03 | 1186.68 | 1187.16 |
| 45 | 3500.96 | 1167.99 | 1168.48 | 194 | 3557.03 | 1186.68 | 1187.35 |
| 46 | 3526.06 | 1176.35 | 1176.80 | 195 | 3421.95 | 1141.66 | 1142.39 |
| 47 | 3476.03 | 1159.68 | 1160.24 | 196 | 3437.94 | 1146.99 | 1147.66 |
| 48 | 3500.96 | 1167.99 | 1168.57 | 197 | 3479.95 | 1160.99 | 1161.82 |
| 50 | 3476.99 | 1160.00 | 1160.24 | 198 | 3421.95 | 1141.66 | 1142.39 |
| 51 | 4114.30 | 1372.43 | 1372.91 | 199 | 3449.99 | 1151 | 1151.73 |
| 52 | 4200.34 | 1401.11 | 1401.77 | 200 | 3508 | 1170.34 | 1170.88 |
| 53 | 4128.32 | 1377.11 | 1377.72 | 201 | 3468 | 1157.01 | 1157.56 |
| 54 | 4169.38 | 1390.79 | 1391.32 | 202 | 3449.95 | 1150.99 | 1151.64 |
| 55 | 4134.32 | 1379.11 | 1379.57 | 203 | 3449.99 | 1151 | 1151.73 |
| 56 | 4170.33 | 1391.11 | 1391.69 | 204 | 3508 | 1170.34 | 1170.97 |
| LP1 | 3918.19 | 1307.07 | 1307.79 | 205 | 3493.98 | 1165.67 | 1166.35 |
| LP2 | 4130.34 | 1377.79 | 1387.18 | 206 | 3197.87 | 1066.96 | 1067.65 |
| LP3 | 4222.33 | 1408.45 | 1408.8 | 207 | 2884.7 | 962.57 | 963.21 |
| LP4 | 4191.3 | 1398.11 | 1398.53 | 208 | 2384.42 | 795.81 | 796.24 |
| LP5 | 4161.32 | 1388.11 | 1388.73 | 209 | 3880.2 | 1294.41 | nd |
| LP6 | 4191.3 | 1398.11 | 1398.91 | 210 | 3904.2 | 1302.41 | 1303.35 |
| LP7 | 4161.32 | 1388.11 | 1388.64 | 211 | 3812.16 | 1271.73 | nd |
| LP8 | 4196.28 | 1399.77 | 1400.2 | 212 | 3549.04 | 1184.02 | 1184.57 |
| LP9 | 4228.32 | 1410.45 | 1410.93 | 213 | 3366.93 | 1123.32 | 1123.89 |
| LP10 | 4167.3 | 1390.11 | 1390.58 | 214 | 3590.09 | 1197.7 | 1198.26 |
| LP11 | 4188.29 | 1397.1 | 1397.61 | 215 | 3531.01 | 1178.01 | 1178.56 |
| LP12 | 4188.29 | 1397.1 | 1397.61 | 216 | 3521.03 | 1174.68 | 1175.32 |
| LP13 | 4060.19 | 1354.4 | 1354.87 | 217 | 3748.16 | 1250.39 | 1251.36 |
| LP14 | 4086.24 | 1363.09 | 1363.57 | 218 | 3591.05 | 1198.02 | 1198.54 |
| LP15 | 3934.15 | 1312.39 | 1312.88 | 219 | 3408.94 | 1137.32 | 1138.04 |
| LP16 | 3933.2 | 1312.07 | 1312.6 | 220 | 3632.1 | 1211.71 | 1212.32 |
| LP17 | 3952.17 | 1318.4 | 1318.8 | 221 | 3573.02 | 1192.01 | 1192.8 |
| LP18 | 3891.15 | 1298.06 | 1298.54 | 222 | 3563.04 | 1188.69 | 1189.38 |
| LP19 | 3949.17 | 1317.4 | 1317.96 | 223 | 3531.01 | 1178.01 | 1179.02 |
| LP20 | 3910.16 | 1304.39 | 1305.01 | 224 | 3587.05 | 1196.69 | 1197.43 |
| LP21 | 3900.15 | 1301.06 | 1301.5 | 225 | 3464.02 | 1155.68 | 1156.36 |
| LP22 | 3907.15 | 1303.39 | 1303.9 | 226 | 3516.04 | 1173.02 | 1173.75 |
| LP23 | 3908.09 | 1303.7 | 1304.27 | 227 | 3516.04 | 1173.02 | 1173.75 |
| LP24 | 3926.12 | 1309.71 | 1310.29 | 228 | 3565.02 | 1189.35 | 1190.03 |
| LP25 | 3892.17 | 1298.4 | 1298.91 | 229 | 3565.02 | 1189.35 | 1190.12 |
| LP26 | 3893.12 | 1298.71 | 1299.19 | 230 | 3560.99 | 1188 | 1189.01 |
| LP27 | 3911.14 | 1304.72 | 1305.29 | 231 | 3570.02 | 1191.01 | 1191.69 |
| LP28 | 3907.15 | 1303.39 | 1303.9 | 232 | 3531.04 | 1178.02 | 1178.74 |
| LP29 | 3908.09 | 1303.7 | 1304.18 | 233 | 3555.04 | 1186.02 | 1186.7 |
| LP30 | 3850.09 | 1284.37 | 1284.75 | 234 | 3548.01 | 1183.68 | nd |
| LP31 | 3926.12 | 1309.71 | 1310.29 | 235 | 3548.01 | 1183.68 | nd |
| LP32 | 3807.05 | 1270.02 | 1270.51 | 236 | 3555.06 | 1186.03 | nd |
| 133 | 4134.34 | 1379.12 | 1379.57 | 237 | 3555.06 | 1186.03 | nd |
| 134 | 4085.31 | 1362.78 | 1363.2 | 238 | 3613.02 | 1205.35 | nd |
| 135 | 4093.32 | 1365.45 | 1365.88 | 239 | 3669.06 | 1224.03 | nd |
| 136 | 4106.31 | 1369.78 | 1370.23 | 240 | 3614.08 | 1205.7 | 1206.4 |
| 137 | 4071.29 | 1358.1 | 1358.57 | 241 | 3698.12 | 1233.71 | 1234.62 |
| 58 | 3491.02 | 1164.67 | 1165.24 | 242 | 3667.15 | 1223.39 | 1224.35 |
| 59 | 3441.99 | 1148.33 | 1148.86 | 243 | 3647.12 | 1216.71 | 1217.41 |
| 60 | 3450.01 | 1151.00 | 1151.55 | 244 | 3656.09 | 1219.7 | 1220.56 |
| 61 | 3462.99 | 1155.33 | 1155.90 | 245 | 3712.13 | 1238.38 | 1239.06 |
| 62 | 3427.97 | 1143.66 | 1144.24 | 246 | 3681.16 | 1228.06 | 1228.79 |
| 140 | 4474.58 | 1492.53 | 1492.98 | 247 | 3689.13 | 1230.72 | 1231.47 |
| 141 | 4418.53 | 1473.85 | 1474.2 | 248 | 3421.05 | 1141.36 | 1142.02 |
| 63 | 3853.24 | 1285.41 | 1285.96 | 249 | 3508 | 1170.34 | 1171.07 |
| 64 | 3797.20 | 1266.73 | 1267.27 | 250 | 3508 | 1170.34 | 1170.97 |
| 65 | 3836.21 | 1279.74 | 1280.22 | 251 | 3508 | 1170.34 | 1171.07 |
| 66 | 3835.22 | 1279.41 | 1279.85 | 252 | 3536 | 1179.67 | 1180.41 |
| 67 | 3779.18 | 1260.73 | 1261.60 | 253 | 3508 | 1170.34 | 1171.07 |
| LP33 | 3468.9 | 1157.31 | 1157.93 | 254 | 3525.04 | 1176.02 | 1176.8 |
| LP36 | 3563.98 | 1189 | 1189.47 | 255 | 3533.02 | 1178.68 | 1179.39 |
| LP39 | 3491.95 | 1164.99 | 1165.52 | 256 | 3718.12 | 1240.38 | 1241.46 |
| LP40 | 3463.94 | 1155.65 | 1156.17 | 257 | 3703.11 | 1235.38 | 1236.1 |
| LP42 | 3445.92 | 1149.65 | 1150.16 | 258 | 3675.1 | 1226.04 | 1226.75 |
| LP43 | 3445.92 | 1149.65 | 1150.07 | 259 | 3675.1 | 1226.04 | 1226.75 |
| LP41 | 3495.94 | 1166.32 | 1166.9 | 260 | 3675.1 | 1226.04 | 1227.03 |
| LP44 | 3391.9 | 1131.64 | 1132.03 | 261 | 3675.1 | 1226.04 | 1227.12 |
| LP50 | 3467.91 | 1156.98 | 1157.28 | 262 | 3703.11 | 1235.38 | 1236.37 |
| LP45 | 3462.92 | 1155.31 | 1155.9 | 263 | 3675.1 | 1226.04 | 1227.03 |
| LP46 | 3419.91 | 1140.98 | 1141.46 | 264 | 3706.16 | 1236.39 | 1237.3 |
| LP47 | 3469.89 | 1157.64 | 1158.21 | 265 | 3700.12 | 1234.38 | 1235.08 |
| LP48 | 3443.88 | 1148.97 | 1149.51 | 266 | 3490.01 | 1164.34 | nd |
| LP49 | 3453.89 | 1152.3 | 1152.94 | 267 | 3464 | 1155.67 | nd |
| LP52 | 3475.99 | 1159.67 | 1160.24 | 268 | 3609.99 | 1204.34 | 1205.11 |
| LP67 | 3510.96 | 1171.33 | 1171.9 | 269 | 4171.18 | 1391.4 | nd |
| LP68 | 3465.96 | 1156.33 | 1156.73 | 270 | 4226.28 | 1409.77 | nd |
| LP69 | 3460.98 | 1154.67 | 1155.25 | 271 | 4299.28 | 1434.1 | nd |
| LP51 | 3432.97 | 1145.33 | 1145.9 | 272 | 4354.38 | 1452.47 | nd |
| LP53 | 3448.93 | 1150.65 | 1151.18 | 273 | 3537.01 | 1180.01 | 1180.87 |
| LP54 | 3436.9 | 1146.64 | 1147.2 | 274 | 3537.01 | 1180.01 | 1180.78 |
| LP55 | 3422.88 | 1141.97 | 1142.48 | 275 | 3521.05 | 1174.69 | nd |
| LP56 | 3483.94 | 1162.32 | 1162.93 | 276 | 3467.01 | 1156.68 | 1157.47 |
| LP57 | 3453.96 | 1152.33 | 1152.75 | 277 | 3493.99 | 1165.67 | 1166.72 |
| LP58 | 3444.93 | 1149.32 | 1149.79 | 278 | 3467.98 | 1157 | 1157.75 |
| LP59 | 3460.92 | 1154.65 | 1155.25 | 279 | 3579.06 | 1194.03 | 1194.84 |
| LP60 | 3410.94 | 1137.99 | 1138.5 | 280 | 3525.01 | 1176.01 | 1176.8 |
| LP61 | 3449.92 | 1150.98 | 1151.27 | 281 | 3522 | 1175.01 | 1175.78 |
| LP62 | 3435.9 | 1146.31 | 1146.83 | 282 | 3467.95 | 1156.99 | 1157.84 |
| LP63 | 3450.91 | 1151.31 | 1151.64 | 283 | 3537.01 | 1180.01 | 1180.78 |
| LP64 | 3443.95 | 1148.99 | 1149.6 | 284 | 3537.01 | 1180.01 | 1181.15 |
| LP65 | 3484.93 | 1162.65 | 1163.2 | 285 | 3513.04 | 1172.02 | nd |
| LP34 | 3448.93 | 1150.65 | 1151.09 | 286 | 3458.99 | 1154 | nd |
| LP35 | 3491.95 | 1164.99 | 1165.42 | 287 | 3513.04 | 1172.02 | nd |
| LP37 | 3448.93 | 1150.65 | 1151.18 | 288 | 3458.99 | 1154 | 1154.79 |
| LP38 | 3483.94 | 1162.32 | 1162.83 | 289 | 3537.01 | 1180.01 | 1181.15 |
| LP70 | 3569 | 1190.67 | 1190.95 | 290 | 3537.01 | 1180.01 | nd |
| 1 | 3434.92 | 1145.98 | 1146.49 | 291 | 3579.06 | 1194.03 | nd |
| 30 | 3433.97 | 1145.66 | 1146.00 | 292 | 3525.01 | 1176.01 | 1177.08 |
| 32 | 3514.98 | 1172.66 | 1173.38 | 293 | 3537.01 | 1180.01 | 1181.15 |
| 37 | 3457.95 | 1153.65 | 1154.32 | 294 | 3513.04 | 1172.02 | 1173.1 |
| LP71 | 3841.07 | 1281.36 | 1281.89 | 295 | 3562.06 | 1188.36 | nd |
| LP72 | 3862.13 | 1288.38 | 1288.82 | 296 | 3520.01 | 1174.34 | nd |
| LP73 | 3977.17 | 1326.72 | 1327.4 | 297 | 3605.07 | 1202.7 | 1203.63 |
| LP74 | 3823.12 | 1275.37 | 1275.78 | 298 | 3647.12 | 1216.71 | 1217.78 |
| 77 | 3813.13 | 1272.04 | 1272.58 | 299 | 3539.03 | 1180.68 | 1181.61 |
| 103 | 3640.98 | 1214.66 | 1215.25 | 300 | 3689.13 | 1230.72 | 1231.75 |
| 74 | 3563.02 | 1188.67 | 1189.06 | 301 | 3612.09 | 1205.04 | 1205.85 |
| 75 | 3678.06 | 1227.02 | 1227.68 | 302 | 3507.03 | 1170.02 | 1170.88 |
| 105 | 3832.20 | 1278.40 | 1278.70 | 303 | 3541.01 | 1181.34 | 1182.26 |
| 109 | 3789.18 | 1264.06 | 1264.21 | 304 | 3507.03 | 1170.02 | 1171.16 |
| 110 | 3790.18 | 1264.39 | 1264.66 | 305 | 3541.01 | 1181.34 | 1182.54 |
| 39 | 3527.02 | 1176.67 | 1177.17 | 306 | 3564.08 | 1189.03 | 1190.21 |
| 41 | 3485.01 | 1162.67 | 1163.20 | 307 | 3598.07 | 1200.36 | 1201.5 |
| 43 | 3491.01 | 1164.67 | 1165.24 | 308 | 3516.03 | 1173.02 | 1174.03 |
| 49 | 3500.96 | 1167.99 | 1168.48 | 309 | 3550.01 | 1184.34 | 1185.5 |
| 70 | 3459.96 | 1154.32 | 1155.25 | 310 | 3516.03 | 1173.02 | 1173.93 |
| 71 | 3586.06 | 1196.35 | 1196.97 | 311 | 3550.01 | 1184.34 | nd |
| 73 | 3529.03 | 1177.34 | 1178.00 | 312 | 3459.01 | 1154.01 | 1154.88 |
| 93 | 3879.24 | 1294.08 | 1294.84 | 313 | 3492.99 | 1165.34 | 1166.26 |
| 69 | 3694.11 | 1232.37 | 1232.89 | 314 | 3450.01 | 1151.01 | 1151.83 |
| 76 | 3710.11 | 1237.70 | 1238.38 | 315 | 3483.99 | 1162.34 | 1163.48 |
| 94 | 3813.16 | 1272.05 | 1272.58 | 316 | 3392.98 | 1132 | 1132.86 |
| 95 | 3847.12 | 1283.37 | 1284.01 | 317 | 3572.09 | 1191.7 | 1192.62 |
| 96 | 3779.18 | 1260.73 | 1261.24 | 318 | 3603.06 | 1202.03 | 1203.17 |
| 97 | 3765.16 | 1256.05 | 1256.92 | 319 | 3739.13 | 1247.38 | 1248.49 |
| 98 | 3894.22 | 1299.07 | 1299.58 | 320 | 3708.16 | 1237.06 | nd |
| 99 | 3813.16 | 1272.05 | 1272.67 | 321 | 3682.11 | 1228.38 | 1229.25 |
| 101 | 3722.15 | 1241.72 | 1242.25 | 322 | 3651.14 | 1218.05 | 1219.17 |
| 102 | 3766.14 | 1256.38 | 1256.74 | 323 | 3682.11 | 1228.38 | 1229.34 |
| 100 | 3738.11 | 1247.04 | 1247.65 | 324 | 3651.14 | 1218.05 | 1218.8 |
| 88 | 3764.20 | 1255.73 | 1256.36 | 325 | 3625.09 | 1209.37 | 1210.29 |
| 89 | 3813.18 | 1272.06 | 1272.64 | 326 | 3594.12 | 1199.05 | 1200.21 |
| 90 | 3894.22 | 1299.07 | 1299.55 | 327 | 3629.11 | 1210.71 | 1211.68 |
| 91 | 3795.14 | 1266.05 | 1266.99 | 328 | 3660.08 | 1221.03 | 1222.13 |
| 92 | 3921.25 | 1308.08 | 1308.71 | 329 | 3673.11 | 1225.38 | 1226.48 |
| 78 | 3828.17 | 1277.06 | 1277.63 | 330 | 3701.14 | 1234.72 | 1235.54 |
| 79 | 3487.07 | 1163.36 | 1163.85 | 331 | 3732.11 | 1245.04 | 1245.9 |
| 83 | 3837.13 | 1280.04 | 1280.40 | 332 | 3715.16 | 1239.39 | 1240.26 |
| 81 | 3855.21 | 1286.07 | 1286.51 | 333 | 3683.13 | 1228.72 | 1229.81 |
| 80 | 3837.13 | 1280.04 | 1280.59 | 334 | 3686.13 | 1229.72 | 1230.45 |
| 85 | 3831.23 | 1278.08 | 1278.56 | 335 | 3717.1 | 1240.04 | 1241.09 |
| 84 | 3831.23 | 1278.08 | 1278.65 | 336 | 3616.09 | 1206.37 | 1207.51 |
| 68 | 3779.18 | 1260.73 | 1261.17 | 337 | 3629.11 | 1210.71 | nd |
| 122 | 3466.98 | 1156.67 | nd | 338 | 3660.08 | 1221.03 | 1222.13 |
| 123 | 3442.05 | 1148.36 | nd | 339 | 3616.09 | 1206.37 | 1207.51 |
| 125 | 3465.96 | 1156.33 | 1156.91 | 340 | 3559.07 | 1187.36 | 1188.18 |
| 125 | 3427.01 | 1143.34 | 1143.87 | 341 | 3426.97 | 1143.33 | 1144.42 |
| 126 | 3441.03 | 1148.02 | 1148.59 | 342 | 3274.94 | 1092.65 | 1093.55 |
| 127 | 3451.95 | 1151.66 | 1152.2 | 343 | 3688.11 | 1230.38 | 1231.19 |
| 128 | 3482.92 | 1161.98 | 1162.56 | 344 | 3754.13 | 1252.38 | 1253.58 |
| 129 | 3457.98 | 1153.67 | 1154.32 | 345 | 3631.09 | 1211.37 | 1212.32 |
| 130 | 3493.96 | 1165.66 | 1166.26 | 346 | 3697.11 | 1233.38 | 1234.25 |
| 131 | 3893.18 | 1298.73 | 1299.37 | 347 | 3631.09 | 1211.37 | 1212.23 |
| 87 | 3795.14 | 1266.05 | 1266.62 | 348 | 3697.11 | 1233.38 | 1234.43 |
| 116 | 3880.2 | 1294.41 | 1294.93 | 349 | 3574.07 | 1192.36 | 1193.54 |
| 117 | 3831.18 | 1278.07 | 1279.02 | 350 | 3640.09 | 1214.37 | 1215.47 |
| 118 | 3831.18 | 1278.07 | 1278.56 | 351 | 3731.14 | 1244.72 | 1245.81 |
| 119 | 3894.19 | 1299.07 | 1299.65 | 352 | 3716.13 | 1239.72 | 1240.54 |
| 120 | 3812.16 | 1271.73 | 1272.45 | 353 | 3688.1 | 1230.37 | 1231.47 |
| 121 | 3926.23 | 1309.75 | 1310.29 | 354 | 3673.09 | 1225.37 | 1226.2 |
| LP75 | 3471.89 | 1158.3 | 1158.86 | 355 | 3745.12 | 1249.38 | 1250.25 |
| LP76 | 3540.96 | 1181.33 | 1219.91 | 356 | 3681.09 | 1228.04 | 1229.16 |
| 144 | 3694.11 | 1232.38 | 1232.95 | 357 | 3854.17 | 1285.73 | nd |
| 145 | 3710.11 | 1237.71 | 1238.22 | 358 | 3716.13 | 1239.72 | 1240.63 |
| 146 | 3813.16 | 1272.06 | 1272.64 | 359 | 3607.03 | 1203.35 | 1204.55 |
| 147 | 3847.12 | 1283.38 | 1284.2 | 360 | 3723.18 | 1242.07 | 1242.94 |
| 148 | 3779.18 | 1260.73 | 1261.07 | 361 | 3573.05 | 1192.02 | 1192.9 |
| 149 | 3765.16 | 1256.06 | 1256.54 | 362 | 3754.14 | 1252.39 | 1253.4 |
| 424 | 3744.15 | 1249.06 | 1249.59 | 363 | 3558.05 | 1187.02 | 1188.18 |
| 412 | 3702.1 | 1235.04 | 1235.64 | 367 | 3473.01 | 1158.68 | 1159.41 |
| 413 | 3735.07 | 1246.03 | 1246.98 | 368 | 3429.99 | 1144.34 | 1145.07 |
| 414 | 3726.1 | 1243.04 | 1243.56 | 369 | 3372.97 | 1125.33 | 1126.11 |
| 415 | 3754.11 | 1252.38 | 1253.29 | 370 | 3372.97 | 1125.33 | 1126.11 |
| 416 | 3674.1 | 1225.71 | 1226.37 | 371 | 3372.97 | 1125.33 | 1126.11 |
| 417 | 3683.06 | 1228.69 | 1229.25 | 372 | 3387.98 | 1130.33 | 1131.1 |
| 418 | 3669.04 | 1224.02 | 1225.01 | 373 | 3551.03 | 1184.68 | 1185.4 |
| 419 | 3683.06 | 1228.69 | 1229.25 | 374 | 3508.01 | 1170.34 | 1171.07 |
| 420 | 3745.14 | 1249.39 | 1249.86 | 375 | 3450.99 | 1151.34 | 1152.01 |
| 421 | 3745.14 | 1249.39 | 1250.2 | 376 | 3450.99 | 1151.34 | 1152.1 |
| 422 | 3745.14 | 1249.39 | 1249.74 | 377 | 3450.99 | 1151.34 | 1152.1 |
| 423 | 3803.13 | 1268.72 | 1269.45 | 378 | 3466 | 1156.34 | 1157.1 |
| 434 | 3738.17 | 1247.06 | 1247.52 | 394 | 3607.1 | 1203.37 | 1203.86 |
| 435 | 3716.14 | 1239.72 | 1240.21 | 407 | 3549.08 | 1184.03 | 1184.68 |
| 441 | 3730.16 | 1244.39 | 1244.83 | 408 | 3578.1 | 1193.71 | 1194.67 |
| 436 | 3732.1 | 1245.04 | 1245.48 | LP95 | 3622.04 | 1208.35 | 1208.9 |
| LP94 | 3739.09 | 1247.37 | 1247.89 | | | | |
| 429 | 3747.13 | 1250.05 | 1250.76 | | | | |

### Example 2

### Potency Assay in SaOS-2 Cells (cAMP)-PTH1 Receptor

Human PTH1 Receptor:

SaOS-2 cells (ATCC, Manassas MD) were maintained in culture in McCoy's 5a medium supplemented with non-essential amino acids (Lifetechnologies, Carlsbad Calif.) and 15% fetal bovine serum (FBS) at 5% CO₂. For potency assays, cells were recovered from culture plates by trypsinization followed by neutralization with complete medium. The cells were pelleted and resuspended in assay buffer (HBSS, 10 mM Hepes, pH 7.3, 0.1% BSA, 0.3% DMSO, and 0.5 mM IBMX) at 1×10⁶ cells/mL. Cells were added to 384 well plates (10K cells per well) and test compounds diluted in assay buffer were added and mixed. Following a 10 minute incubation (room temperature), human PTH[1-34] (Bachem, Torrance Calif.) was added at a final concentration of 2 nM (approximately EC₉₀) to stimulate the PTH1 receptor. After 30 minutes, cAMP concentrations were determined for each well using an HTRF based kit according to the manufacturer's instructions (CisBio, Bedford Mass.). Concentrations of cAMP vs log concentration of test compound were plotted and a four-parameter curve fit was used to calculate an IC₅₀ (GraphPad, La Jolla Calif.) for each compound. The following legend is used in Table 11 shown below: IC₅₀: <40 nM (“++++”), 41-700 nM (“+++”), 701-1500 nM (“++”), >1500 nM (“+”); Kᵢ: <2 nM (“+++”), 2-50 nM (“++”), >50 nM (“+”).

TABLE 11

| | | | | | | PTH1 | | | | PTH1 | |
|---|---|---|---|---|---|---|---|---|---|---|---|
| | | PTH1 | | | | IC₅₀ 10 | | | | IC₅₀ 10 | |
| | SaOS | IC₅₀ 10 | | | SaOS | nM | | | SaOS | nM | |
| | hPTH1 | nM PTH | SaOS | SP# | hPTH1 | PTH | SaOS | SP# | hPTH1 | PTH | SaOS |
| SP# or | IC₅₀ | (PTH | Kᵢ | or | IC₅₀ | (PTH | Kᵢ | or | IC₅₀ | (PTH | Kᵢ |
| compd | (nM) | SaOS2) | (nM) | compd | (nM) | SaOS2) | (nM) | compd | (nM) | SaOS2) | (nM) |
| BIM- | +++ | +++ | ++ | 95 | ++++ | ++++ | +++ | 243 | ++++ | ++++ | +++ |
| 44002 | | | | | | | | | | | |
| Ac- | +++ | +++ | ++ | 96 | ++++ | ++++ | +++ | 244 | ++++ | ++++ | +++ |
| BIM- | | | | | | | | | | | |
| 44002 | | | | | | | | | | | |
| 600 | | +++ | ++ | 97 | ++++ | ++++ | +++ | 245 | ++++ | ++++ | +++ |
| 601 | | ++++ | +++ | 98 | ++++ | +++ | ++ | 246 | ++++ | ++++ | +++ |
| 602 | +++ | | | 99 | ++++ | ++++ | ++ | 247 | ++++ | ++++ | +++ |
| 603 | +++ | | | 101 | ++++ | ++++ | +++ | 248 | ++++ | +++ | ++ |
| 604 | +++ | | | 102 | ++++ | ++++ | +++ | 249 | ++++ | | ++ |
| 57 | ++++ | | ++ | 100 | ++++ | ++++ | +++ | 250 | +++ | | + |
| 112 | +++ | +++ | ++ | 88 | ++++ | | ++ | 251 | ++++ | | ++ |
| 113 | +++ | +++ | ++ | 89 | ++++ | | ++ | 252 | ++++ | | +++ |
| 114 | ++++ | ++++ | +++ | 90 | ++++ | | ++ | 253 | ++++ | | +++ |
| 2 | +++ | | + | 91 | ++++ | | ++ | 254 | ++++ | | ++ |
| 3 | +++ | | + | 92 | ++++ | +++ | ++ | 255 | ++++ | | ++ |
| 4 | + | | + | 78 | ++++ | ++++ | +++ | 257 | ++++ | | +++ |
| 5 | +++ | | + | 79 | ++++ | ++++ | +++ | 258 | ++++ | | +++ |
| 6 | ++ | | + | 83 | ++++ | +++ | ++ | 259 | ++++ | | +++ |
| 7 | +++ | | + | 81 | ++++ | | ++ | 260 | ++++ | | ++ |
| 8 | +++ | | + | 80 | ++++ | ++++ | +++ | 261 | ++++ | | +++ |
| 9 | +++ | | + | 85 | ++++ | | ++ | 262 | ++++ | | +++ |
| 10 | ++++ | | ++ | 84 | ++++ | | ++ | 263 | ++++ | | +++ |
| 11 | +++ | | + | 68 | ++++ | | +++ | 264 | ++++ | | +++ |
| 12 | +++ | | + | 125 | ++++ | | ++ | 265 | ++++ | | +++ |
| 13 | ++++ | | ++ | 126 | +++ | | ++ | 607 | | | +++ |
| 14 | ++++ | | ++ | 127 | +++ | | ++ | 268 | +++ | + | + |
| 15 | ++++ | | ++ | 128 | +++ | | + | 273 | ++++ | ++++ | +++ |
| 16 | ++++ | | ++ | 130 | +++ | | + | 274 | ++++ | ++++ | +++ |
| 17 | ++++ | | ++ | 86 | ++++ | +++ | ++ | 276 | ++++ | ++++ | +++ |
| 18 | ++++ | | ++ | 87 | +++ | | + | 277 | ++++ | ++++ | +++ |
| 19 | ++++ | | ++ | 116 | ++++ | | ++ | 278 | ++++ | ++++ | +++ |
| 21 | ++++ | | +++ | 117 | ++++ | | +++ | 279 | ++++ | ++++ | +++ |
| 22 | +++ | | | 118 | ++++ | | ++ | 280 | ++++ | ++++ | +++ |
| 23 | +++ | | | 119 | ++++ | | ++ | 281 | ++++ | ++++ | +++ |
| 24 | + | | | 121 | +++ | | + | 282 | ++++ | ++++ | +++ |
| 28 | ++ | | + | LP75 | ++++ | | ++ | 283 | ++++ | ++++ | +++ |
| 29 | +++ | | + | LP76 | ++++ | | +++ | 284 | ++++ | ++++ | +++ |
| 31 | + | | + | 144 | +++ | +++ | + | 286 | ++++ | ++++ | +++ |
| 33 | +++ | | + | 145 | ++++ | ++++ | +++ | 288 | ++++ | ++++ | +++ |
| 40 | ++++ | | ++ | 146 | ++++ | ++++ | +++ | 289 | ++++ | ++++ | +++ |
| 42 | ++++ | | ++ | 147 | ++++ | ++++ | ++ | 292 | ++++ | ++++ | +++ |
| 50 | +++ | | ++ | 148 | ++++ | ++++ | +++ | 293 | ++++ | +++ | ++ |
| LP1 | ++ | | | 149 | ++++ | ++++ | +++ | 294 | ++++ | +++ | ++ |
| LP3 | | ++++ | | 150 | ++++ | ++++ | ++ | 297 | ++++ | +++ | ++ |
| LP4 | ++++ | | ++ | 151 | ++++ | ++++ | ++ | 298 | ++++ | +++ | +++ |
| LP5 | +++ | | | 152 | ++++ | ++++ | +++ | 299 | ++++ | ++++ | +++ |
| LP6 | ++++ | | ++ | 153 | ++++ | ++++ | +++ | 300 | ++++ | | +++ |
| LP7 | +++ | | | 154 | ++++ | ++++ | ++ | 301 | ++++ | | +++ |
| LP8 | +++ | | | 155 | ++++ | | ++ | 302 | ++++ | | +++ |
| LP9 | ++++ | | ++ | 156 | +++ | | ++ | 303 | ++++ | | ++ |
| LP10 | +++ | | ++ | 157 | ++++ | | ++ | 304 | ++++ | | +++ |
| LP11 | ++++ | | | 158 | ++++ | | +++ | 305 | ++++ | | ++ |
| LP18 | +++ | | + | 159 | ++++ | | ++ | 306 | ++++ | | +++ |
| LP19 | +++ | | + | 160 | ++++ | ++++ | +++ | 307 | ++++ | | +++ |
| LP20 | +++ | | ++ | 161 | ++++ | ++++ | +++ | 308 | ++++ | | +++ |
| LP31 | ++++ | | ++ | 162 | ++++ | ++++ | +++ | 309 | ++++ | | ++ |
| LP32 | +++ | | + | 163 | ++++ | +++ | ++ | 310 | ++++ | | +++ |
| 58 | +++ | | ++ | 164 | ++++ | ++++ | +++ | 311 | ++++ | | +++ |
| 59 | +++ | | + | 165 | ++++ | ++++ | +++ | 312 | ++++ | | ++ |
| 60 | +++ | | + | 166 | ++++ | +++ | ++ | 313 | ++++ | | +++ |
| 61 | ++++ | +++ | ++ | 167 | ++++ | ++++ | +++ | 314 | ++++ | | ++ |
| 62 | +++ | | ++ | 168 | ++++ | +++ | ++ | 315 | ++++ | | ++ |
| 63 | ++ | | + | 169 | ++++ | ++++ | +++ | 316 | ++++ | | ++ |
| 64 | +++ | | + | 170 | ++++ | ++++ | +++ | 317 | ++++ | | +++ |
| 82 | ++++ | | | 171 | ++++ | ++++ | +++ | 318 | ++++ | | +++ |
| 65 | +++ | | + | 172 | ++++ | ++++ | +++ | 321 | ++++ | | +++ |
| 66 | +++ | | + | 173 | ++++ | ++++ | +++ | 322 | ++++ | | +++ |
| 67 | ++++ | ++++ | | 174 | ++++ | ++++ | +++ | 323 | ++++ | | ++ |
| LP33 | ++++ | | ++ | 175 | ++++ | ++++ | ++ | 324 | ++++ | | ++ |
| LP36 | ++++ | | +++ | 176 | ++++ | ++++ | +++ | 325 | ++++ | | +++ |
| LP39 | ++++ | | +++ | 177 | ++++ | | ++ | 326 | ++++ | | ++ |
| LP40 | ++++ | | ++ | 178 | ++++ | ++++ | +++ | 327 | ++++ | | +++ |
| LP42 | ++++ | | ++ | 179 | ++++ | ++++ | ++ | 328 | ++++ | | +++ |
| LP43 | +++ | | + | 180 | ++++ | | ++ | 329 | ++++ | | +++ |
| LP41 | ++++ | ++++ | ++ | 181 | ++++ | | ++ | 330 | ++++ | | +++ |
| LP44 | +++ | | + | 182 | ++++ | ++++ | +++ | 331 | ++++ | | +++ |
| LP50 | ++ | | | 183 | ++++ | | ++ | 332 | ++++ | | +++ |
| LP45 | +++ | | + | 184 | ++++ | ++++ | +++ | 333 | ++++ | | +++ |
| LP46 | ++ | ++++ | | 185 | ++++ | ++++ | +++ | 334 | ++++ | | +++ |
| LP47 | ++ | ++++ | | 186 | ++++ | ++++ | +++ | 335 | ++++ | | +++ |
| LP48 | +++ | ++++ | | 187 | ++++ | ++++ | +++ | 336 | ++++ | | +++ |
| LP49 | ++ | ++++ | | 188 | ++++ | ++++ | +++ | 338 | ++++ | | +++ |
| LP52 | +++ | | + | 189 | ++++ | +++ | +++ | 339 | ++++ | | +++ |
| LP67 | +++ | | + | 190 | ++++ | | ++ | 340 | ++++ | | +++ |
| LP68 | ++++ | +++ | ++ | 191 | ++++ | ++++ | +++ | 341 | ++++ | | ++ |
| LP69 | +++ | +++ | ++ | 192 | ++++ | | +++ | 342 | ++++ | | +++ |
| LP51 | +++ | | + | 193 | ++++ | | +++ | 343 | ++++ | | +++ |
| LP53 | +++ | | + | 194 | ++++ | ++++ | +++ | 344 | ++++ | | +++ |
| LP54 | +++ | | + | 195 | ++++ | +++ | ++ | 345 | ++++ | | +++ |
| LP55 | +++ | ++++ | + | 196 | ++++ | | ++ | 346 | ++++ | | ++ |
| LP56 | +++ | | + | 197 | +++ | | + | 347 | ++++ | | +++ |
| LP57 | +++ | | + | 198 | ++++ | +++ | ++ | 348 | ++++ | | +++ |
| LP58 | +++ | | + | 199 | ++++ | ++++ | +++ | 349 | ++++ | | +++ |
| LP59 | ++++ | +++ | ++ | 200 | ++++ | ++++ | +++ | 350 | ++++ | | +++ |
| LP60 | +++ | | + | 201 | ++++ | ++++ | +++ | 351 | ++++ | | +++ |
| LP61 | +++ | | + | 202 | ++++ | ++++ | +++ | 353 | ++++ | | +++ |
| LP62 | +++ | ++++ | | 203 | ++++ | ++++ | +++ | 354 | ++++ | | +++ |
| LP63 | +++ | ++++ | | 204 | ++++ | ++++ | +++ | 355 | ++++ | | +++ |
| LP64 | +++ | | + | 205 | ++++ | | +++ | 356 | ++++ | | +++ |
| LP65 | +++ | | + | 206 | ++++ | +++ | ++ | 358 | | | +++ |
| LP34 | +++ | | + | 210 | ++++ | +++ | ++ | 359 | ++++ | | +++ |
| LP35 | +++ | | + | 606 | | +++ | ++ | 360 | ++++ | | +++ |
| LP37 | ++++ | | ++ | 211 | ++++ | | ++ | 361 | ++++ | | +++ |
| LP38 | ++++ | +++ | ++ | 212 | ++++ | | +++ | 362 | ++++ | | +++ |
| LP70 | ++++ | ++++ | ++ | 213 | ++++ | | ++ | 363 | | | +++ |
| 605 | +++ | ++++ | ++ | 214 | ++++ | | +++ | 367 | | | +++ |
| 1 | +++ | | + | 215 | ++++ | | +++ | 368 | | | ++ |
| 30 | +++ | | + | 216 | ++++ | | +++ | 369 | | | ++ |
| 32 | + | | + | 217 | ++++ | ++++ | +++ | 370 | | | +++ |
| LP71 | ++++ | ++++ | +++ | 218 | ++++ | | +++ | 371 | | | ++ |
| LP72 | ++++ | ++++ | +++ | 219 | ++++ | | ++ | 372 | | | +++ |
| LP74 | ++++ | ++++ | +++ | 220 | ++++ | | +++ | 373 | | | +++ |
| 77 | ++++ | ++++ | +++ | 221 | ++++ | | +++ | 374 | | | ++ |
| 103 | +++ | + | + | 222 | ++++ | | +++ | 375 | | | ++ |
| 74 | ++++ | ++++ | +++ | 223 | ++++ | +++ | ++ | 376 | | | ++ |
| 75 | ++++ | +++ | +++ | 224 | ++++ | + | ++ | 377 | | | ++ |
| 105 | | ++++ | ++ | 225 | ++++ | +++ | ++ | 378 | | | +++ |
| 109 | +++ | +++ | + | 226 | ++++ | ++++ | +++ | 69 | +++ | +++ | ++ |
| 110 | ++++ | ++++ | ++ | 227 | ++++ | +++ | ++ | 76 | ++++ | ++++ | ++ |
| 41 | ++ | | + | 228 | ++++ | ++++ | +++ | 94 | ++++ | ++++ | +++ |
| 43 | ++++ | | ++ | 229 | ++++ | +++ | ++ | 240 | ++++ | ++++ | +++ |
| 70 | ++++ | +++ | ++ | 230 | ++++ | ++++ | +++ | 241 | ++++ | ++++ | +++ |
| 71 | ++++ | ++++ | +++ | 231 | ++++ | ++++ | +++ | 242 | ++++ | ++++ | +++ |
| 73 | ++++ | ++++ | ++ | 232 | ++++ | ++++ | +++ | | | | |
| 93 | +++ | | + | 233 | ++++ | ++++ | +++ | | | | |

### Rat PTH1 Receptor:

Assays for rat PTH1 receptor were performed as for human except UMP-106 cells were used. UMP-106 were cultured in DMEM supplemented with 10% FBS (Lifetechnologies). The following legend is used for Table 12 below: IC₅₀: <50 nM (“++++”), 51-250 nM (“+++”), 251-2000 nM (“++”), >2001 nM (“+”); Kᵢ: <2 nM (“+++”), 2-20 nM (“++”), >20 nM (“+”).

TABLE 12

| | Rat | Rat | | Rat | Rat | | Rat | Rat |
|---|---|---|---|---|---|---|---|---|
| | PTH1 | PTH1 | | PTH1 | PTH1 | | PTH1 | PTH1 |
| | IC₅₀ | Kᵢ | | IC₅₀ | Kᵢ | | IC₅₀ | Kᵢ |
| SP# | (nM) | (nM) | SP# | (nM) | (nM) | SP# | (nM) | (nM) |
| BIM-44002 | ++ | + | 184 | ++++ | ++ | 252 | ++++ | ++ |
| Ac-BIM- | ++ | + | 185 | ++++ | +++ | 253 | ++++ | +++ |
| 44002 | | | | | | | | |
| 608 | ++ | + | 186 | ++++ | +++ | 254 | ++++ | ++ |
| 609 | +++ | + | 187 | ++++ | +++ | 255 | +++ | + |
| 602 | + | + | 188 | ++++ | ++ | 257 | ++++ | +++ |
| 603 | ++ | + | 189 | ++++ | ++ | 258 | ++++ | +++ |
| 604 | +++ | + | 199 | ++++ | +++ | 259 | ++++ | ++ |
| 57 | + | + | 200 | ++++ | ++ | 262 | ++++ | ++ |
| 114 | ++++ | ++ | 201 | ++++ | + | 263 | ++++ | +++ |
| 58 | + | + | 202 | ++++ | ++ | 276 | ++++ | +++ |
| 59 | + | + | 203 | ++++ | +++ | 277 | ++++ | ++ |
| 60 | + | + | 204 | ++++ | ++ | 278 | ++++ | ++ |
| 61 | + | + | 205 | ++++ | ++ | 280 | ++++ | ++ |
| 62 | + | + | 212 | +++ | + | 281 | ++++ | ++ |
| 63 | + | + | 213 | ++++ | ++ | 282 | ++++ | ++ |
| 64 | + | + | 214 | ++++ | + | 283 | ++++ | +++ |
| 65 | + | + | 215 | ++++ | ++ | 288 | ++++ | +++ |
| 66 | +++ | + | 217 | ++++ | ++ | 292 | ++++ | +++ |
| 67 | ++++ | ++ | 218 | ++++ | + | 299 | ++++ | +++ |
| 70 | +++ | + | 220 | ++++ | ++ | 306 | ++++ | +++ |
| 71 | ++++ | ++ | 221 | ++++ | ++ | 307 | ++++ | +++ |
| 73 | ++++ | + | 226 | ++++ | ++ | 330 | ++++ | ++ |
| 76 | +++ | + | 228 | ++++ | +++ | 343 | ++++ | +++ |
| 79 | ++++ | ++ | 230 | ++++ | ++ | 345 | ++++ | ++ |
| 80 | ++++ | + | 232 | ++++ | +++ | 347 | ++++ | +++ |
| 145 | ++++ | ++ | 240 | ++++ | + | 351 | ++++ | +++ |
| 160 | ++++ | ++ | 241 | ++++ | ++ | 353 | ++++ | +++ |
| 161 | ++++ | ++ | 242 | ++++ | ++ | 354 | ++++ | +++ |
| 162 | ++++ | ++ | 243 | ++++ | ++ | 359 | ++++ | +++ |
| 172 | ++++ | ++ | 244 | ++++ | ++ | 360 | ++++ | ++ |
| 173 | ++++ | ++ | 245 | ++++ | ++ | 361 | ++++ | +++ |
| 182 | ++++ | ++ | 246 | ++++ | ++ | 363 | ++++ | ++ |
| 183 | ++++ | +++ | 247 | ++++ | +++ | 378 | ++++ | ++ |

### Example 3

### [¹²⁵I]-PTH Binding Assay with Parathyroid Hormone Receptors

### Reagents

[¹²⁵I]PTH (1-34): 2200 Ci/mmol, Cat. No.: NEX397010UC, Lot. No.: KF11130; PerkinElmer;
PTH (1-34); Cat. No.: RP01001, Lot. No.: P11611212 GenScript
TIP39; Cat. No.: RP20322, Lot. No.: P11621212 GenScript
Recombinant human PTHR1 cell line; Cat. No.: M00315 GenScript
Recombinant human PTHR2 cell line; Cat. No.: M00270 GenScript
BSA: Cat. No # A7901 Sigma
Binding buffer: 20 mM HEPES, 100 mM NaCl, 3 mM MgCl₂, 1 mM EDTA, 0.3% BSA, pH 7.4, stored at 4° C.

### Materials

UniFilter-96 GF/C filter plates; Cat. No.#6005177PerkinElmer
Microplate thermo shaker; MB100-4P AoSheng
TopCountRNXT™ Microplate scintillation and luminescence countersPerkinElmer
96 Well clear flat bottom polystyrene TC-treated microplates, #3599Corning
Centrifuge: Model No. Avanti-J-26XP, Rotor: JA-25.50 Beckman

### Assay:

Cell membranes are prepared using GenScript in-house developed stable cell line expressing PTHR and are applied to the binding assay. Membranes are prepared by adding [¹²⁵]PTH (1-34) and cold ligand solution into the 96-well plate and incubated for 90 minutes at 25° C. with a shaking speed of 330 RPM. Each well of the Uni-filter 96 GF/C microplate is pre-wetted with 100 μL binding buffer at 4° C. for 30 min. The reaction system is manually transferred into the filter plates and filtered with Millipore vacuum manifold (8-15 mmHg). The wells are manually washed with 2 ml/well (100 μL×20) cold wash buffer and dried in hood at RT for 60 minutes. The bottoms of the plates are sealed with Bottom Seal™ (opaque) (Perkin Elmer). 50 μl MicroScint 20™ (Perkin Elmer) is added to each well. The plates are sealed with TopSeal A (Perkin Elmer) and counted on TopCount NXT for 1 min/well. Data is recorded by TopCount NXT and stored on the GenScript computer network for off-line analysis. Data acquisition is performed by Microsoft Excel (version 2003) program. Competition binding is calculated by

“Competition %=100*(Total binding-Sample CPM)/(Total binding−NSB).

TABLE 13

| | | | Cold | | |
|---|---|---|---|---|---|
| Reaction | Reaction Vol. | MemPrep | ligand | [¹²⁵I]PTH | Buffer |
| system | (μL) | (μL) | (μL) | (μL) | (μL) |
| Total | 100 | 25 | 0 | 50 | 25 |
| binding | | | | | |
| NSB | 100 | 25 | 25 | 50 | 0 |

The following legend is used for Table 14 below: <0.05 M (“++++”), 0.05-0.09 M (“+++”), 0.1-1 M (“++”), >1 M (“+”).

TABLE 14

| SP# | K_{d} (μM) | SP# | K_{d} (μM) | SP# | K_{d} (μM) |
|---|---|---|---|---|---|
| BIM-44002 | ++ | 73 | ++ | 204 | ++++ |
| 601 | ++ | 711 | ++ | 211 | ++ |
| 609 | ++++ | 76 | ++ | 212 | ++++ |
| 114 | ++++ | 79 | ++++ | 213 | ++++ |
| 61 | + | 80 | ++ | 214 | +++ |
| 63 | ++++ | 86 | ++ | 215 | ++ |
| 67 | ++++ | 120 | ++ | 216 | ++++ |
| LP39 | ++++ | 145 | ++++ | 221 | ++ |
| 710 | ++++ | 160 | +++ | 222 | ++++ |
| 1 | ++ | 161 | ++++ | 226 | ++++ |
| LP72 | ++++ | 182 | ++++ | 228 | ++++ |
| 77 | +++ | 183 | +++ | 230 | +++ |
| 105 | +++ | 186 | ++++ | 232 | ++++ |
| 70 | + | 188 | +++ | 243 | ++++ |
| 71 | ++ | 200 | +++ | 247 | ++++ |
| 71 | ++++ | 201 | +++ | | |

### Example 4

### Potency Assay in CHO Cells (cAMP)-PTH1 Receptor

CHO cells transfected with and stably expressing human PTH1 receptor and Gₐₗₚₕₐ₁₅were obtained from GenScript (Piscataway N.J.) and cultured according to manufacturer's instructions. Antagonist assays were performed as for SaOS-2 cell assays. The following legend is used for Table 15: <250 nM (“++++”), 251-750 nM (“+++”), 751-3700 nM (“++”), >3701 nM (“+”).

TABLE 15

| SP# | CHO/PTH1 | SP# | CHO/PTH1 | SP# | CHO/PTH1 |
|---|---|---|---|---|---|
| 28 | + | 42 | ++++ | 30 | ++ |
| 31 | + | 44 | + | 32 | + |
| 33 | ++ | 45 | + | 39 | + |
| 34 | + | 46 | + | 41 | ++++ |
| 35 | + | 47 | ++ | 43 | ++ |
| 38 | + | 48 | ++ | 49 | + |
| 40 | ++++ | 50 | ++ | | |

### Example 5

### Tag-Lite PTH1R/PTHR2 Binding Assay

The Tag-lite® ligand binding assay is based on the competition between the Tag-lite fluorescent ligand and compounds. The assay is carried out on cells which are expressing the receptor of interest. The interaction between the labelled receptor and the fluorescent ligand is quantified by the FRET signal.

Reagents used in the assay include Tag-lite buffer (5×); PTH receptors red agonist; Nle8,18-Tyr34 PTH (3-34) amide to determine nonspecific signal, and Tag-lite ready-to-use cells (transformed & labelled) PTHR1. To determine the K_{d}, a standard protocol for 20 μL final volume uses the following reagents (Table 16):

TABLE 16

| | Total signal | Non specific signal |
|---|---|---|
| Cell preparation | 10 μL | 10 μL |
| Tag-lite buffer 1X (TLB 1X) | 5 μL | — |
| Unlabeled ligand | — | 5 μL |
| Fluorescent ligand (dose response | 5 μL | 5 μL |
| from F1 to F11) | | |

The reaction is incubated at RT for 1 hr and read on an HTRF compatible reader. 1× Tag lite buffer (TLB) is prepared and the fluorescent ligand was prepared in the TLB. To prepare the fluorescent ligand preparation in TLB 1×, the concentration of fluorescent ligand PTH receptors red agonist indicated on the vial label (=13.21 μM) are used. A fluorescent ligand dilution is prepared by centrifuging the vial then diluting the fluorescent ligand PTH receptors red agonist with TLB 1× in order to obtain the high concentration F1=4800 nM for the top of the K_{d} curve (e.g., take 58.2 μL of fluorescent ligand stock solution and add it to 101.8 μL of TLB 1×). The F1 solution is used to prepare the K_{d} curve using 0.5 serial dilutions in TLB 1× as follows: 100 μL of TLB 1× in each vial is dispensed from F2 to F11. 100 μL of F1 is added to 100 μL of TLB 1×, mixed gently and the 0.5 serial dilution is repeated to make F2, F3, F4, F5, F6, F7, F8, F9, F10, F11 as indicated in Table 17 below.

TABLE 17

Fluorescent ligand concentration (nM)

| | Initial concentrations | Final concentrations |
|---|---|---|
| | (working solutions) | (in the well) |
| F1 | 4800 | 1200 |
| F2 | 2400 | 600 |
| F3 | 1200 | 300 |
| F4 | 600 | 150 |
| F5 | 300 | 75 |
| F6 | 150 | 37.5 |
| F7 | 75 | 18.75 |
| F8 | 37.5 | 9.4 |
| F9 | 18.8 | 4.7 |
| F10 | 9.4 | 2.3 |
| F11 | 4.7 | 1.2 |

To check the specificity of the binding between the fluorescent ligand and labelled receptor, a negative control needs to be run. In this negative control, the binding of the fluorescent ligand onto the receptor is avoided by the addition of a large excess of non-fluorescent ligand. For each concentration of fluorescent-ligand, the nonspecific binding signal is determined using a large excess of unlabeled ligand.

Nle-8,18-Tyr34 PTH (3-34) amide was used as unlabeled ligand.

Prepare a working solution of unlabeled ligand in TLB 1× at 120 μM.

Cell Preparation with TLB 1×

Prepare a conical vial (A) containing 5 mL of cold TLB 1×. Labeled frozen cells are thawed at 37° C. (water bath, manual shaking) until all the ice is thawed (1-2 min) and transferred quickly by pipetting into a vial containing a working solution of unlabeled ligand in TLB 1× at 120 μM. The vial is then centrifuged 5 min at 1200 G at 4° C. The supernatant is gently removed by aspiration. The pellet is resuspended in 1 ml of TLB 1×, and mixed gently by aspiration. 1.2 mL of TLB 1× is added, and mixed gently by aspiration.

### Compound Kᵢ Determination

For the competition dose-response of compounds, the optimal fluorescent ligand concentration is the one that allows 50% (Kᵢ) to 80% of receptor binding. A standard protocol for 20 μL final volume is performed using the reagents indicated in Table 18 below:

TABLE 18

| | Ligand binding |
|---|---|
| Cell preparation | 10 μL |
| Compounds (dose response) | 5 μL |
| Fluorescent ligand at optimal concentration (1X K_{d}) | 5 μL |

The reaction is incubated at RT for 1 hr and read on an HTRF compatible reader. A K_{d} determination protocol as above is used to prepare fluorescent ligand and cells.

### Example 6

### Agonism/Antagonism Activity on PTHR1/PTHR2 (Ca Flux Assays)

PTH2R is a member of the G-protein coupled receptor family. This protein is a receptor for parathyroid hormone (PTH). This receptor is more selective in ligand recognition and has a more specific tissue distribution compared to parathyroid hormone receptor 1 (PTH1R). It is activated only by PTH and not by parathyroid hormone-like hormone (PTHLH) and is particularly abundant in brain and pancreas.

Inhibitory activities of compounds were measured on PTH receptor type 2, and was also used to test agonist activity on PTH receptor type 1 using a calcium flux assay method. The receptor was stimulated with TIP-39 at EC₈₀ concentration (4.1 nM). The IC₅₀ value of PTHrP as control antagonist was 16 μM.

Control articles were prepared as shown below. The stock solutions were diluted in HBSS buffer (with 20 mM HEPES buffer, pH 7.4) to make 5× final concentration solutions. The final concentration of DMSO was 1%. Information about control articles is shown in Table 19:

TABLE 19

| | | | M. Wt | Stock solution | Purity | Storage |
|---|---|---|---|---|---|---|
| Control Article | Vendor | Cat No. | (g/mol) | (DMSO) | (%) | condition |
| TIP-39 | Genscript | 88803108 | 4504.2 | 10 mM | >95 | −20 degree |
| PTHrP-7-34 | Genscript | 209537 | 3364.9 | 10 mM | >99 | −20 degree |

Other reagents used are shown below in Table 20:

TABLE 20

| | | Catalog | Accession Number |
|---|---|---|---|
| Name | Vendor | Number | for receptor |
| CHO-k1/PTH2R/Gα15 | Genscript | N/A | NM_005048 |
| FLIPR ® Calcium 4 | Molecular | R8141 | N/A |
| assay kit | devices | | |
| Probenecid | Sigma | P8761 | N/A |

CHO-k1 cells expressing PTH receptor type 2 were seeded in a 384-well plate at density of 20,000 cells per well in 20 μL of growth medium, 18 hours prior to the day of experiment and maintained at 37° C./5% CO2. CHO-k1/PTH2R/Gα15 cells were regularly subcultured in order to maintain optimal cell health and are cultured in DMEM/F12 1:1 supplemented with 10% fetal bovine serum, 100 μg/mL Hygromycin B and 200 μg/mL zeocin. For the antagonist assay, 20 μL of dye-loading solution and 10 μL of compound solution or control antagonist was added into the well. The plate was then placed into a 37° C. incubator for 60 minutes, followed by 15 minutes at room temperature. At last, 12.5 μL of control agonist was added into respective wells of the assay plate.

Test compounds were prepared and stored at −20° C. The test compounds were diluted in DMSO to make 10 mM stock solutions. The stock solutions were diluted in HBSS buffer (with 20 mM HEPES buffer, pH 7.4) to make 500 μM solutions. Compounds were tested in duplication. The final concentration of DMSO was 1%.

The following EC₅₀ and IC₅₀ values were obtained for reference compounds (Table 21).

TABLE 21

| Reference compound | GenScript values(μM) | Reference values(μM) |
|---|---|---|
| TIP-39(agonist) | 0.00255 | 0.002 |
| PTHrP-7-34(antagonist) | 16 | 1 |

Antagonism activities of 2 compounds on PTH2R are shown in Table 22 (“+” represents IC50>10 μM; “++” represents IC50<10 μM):

TABLE 22

| SP# | IC₅₀ (μM) |
|---|---|
| BIM-44002 | + |
| 67 | ++ |

### Example 7

### Effect of Peptidomimetic Macrocycles on Serum Calcium

The effects of Cinacalcet, BIM-44002, and SP67 on serum calcium in male rats was tested. Cinacalcet was administered orally at two dose levels (10 and 30 mg/kg) and SP67 were dosed IV at two dose levels (1 and 3 mg/kg). BIM-44002 were dosed IV at 2.85 mg/kg. In addition, there was a vehicle control group dosed IV.

Information for the test articles is summarized below.
1. Identity: Cinacalcet HCl
Lot Number: Lot 1
Supplied As: S olid powder
Storage Conditions: −20° C.
Molecular weight: 393.87 (HCl), 357.4 free base
Salt correction factor: No correction applied
2. Identity: BIM-44002
Lot Number: Lot 3
Supplied As: Solid powder
Storage Conditions: −20° C.
Exact mass: 3586.94
Peptide content correction factor: No correction applied
3. Identity: SP-67 (acetate salt)
Lot Number: Lot 3
Supplied As: Solid powder
Storage Conditions: −20° C.
Exact mass: 3779.18
Peptide content correction factor: No correction applied
   The test system for this study was 24 male Sprague Dawley rats, surgically prepared by the supplier. A limited number of additional animals were available as possible replacements for animals not dosed properly. Animals placed on study were assigned an identification number and designated as such with a permanent marker, while unused spare animals were returned to stock after successful dose administration of animals in the study design. Animal information is summarized below.
Species: Rattus norvegicus; Strain: Sprague Dawley
Total No. of Animals: 24 males total, all will be jugular vein cannulated (JVC) for serial blood collections, and 16 will also be femoral vein cannulated (FVC) for IV dosing
Body Weight Range: Approximately 250-275 g at treatment
Acclimation Period: A minimum of 2 days
Fasting: Fasted overnight (approximately 16 h) before dosing and until 4 h post-dose
The above are the intended body weight specifications of the animals used in the experiment. Actual body weights were documented.

### Dose Groups

Animals were assigned to one of six dose groups and each animal was administered a single IV or PO dose of one test article or control vehicle as described in Table 23.

TABLE 23

| | | | Dose | Dose Conc. | Dose Volume | Dose Level |
|---|---|---|---|---|---|---|
| Group | N | Test Article | Route | (mg/mL)* | (mL/kg) | (mg/kg)* |
| 1 (JVC/PVC) | 4 | Control (vehicle) | IV | NA | 20 | NA |
| 2 (JVC) | 4 | Cinacalcet | PO | 15 | 2 | 30 |
| 3 (JVC) | 4 | Cinacalcet | PO | 5 | 2 | 10 |
| 4 (JVC/PVC) | 4 | BIM-44002 | IV | 0.285 | 10 | 2.85 |
| 5 (JVC/PVC) | 4 | SP-67 | IV | 0.15 | 20 | 3 |
| 6 (JVC/PVC) | 4 | SP-67 | IV | 0.15 | 6.67 | 1 |

*Dose concentrations and dose levels were not corrected for peptide content or salt concentration

### Dosing Formulations

Dosing formulations were prepared within 24 h of dosing. The dosing formulations were prepared to contain the test article concentrations indicated in the table above. Sponsor pre-weighed or weighed test article were mixed with the appropriate dosing vehicle and sonicated, if necessary, to produce solutions for IV dosing and solutions or suspensions for PO dosing. The dosing formulations were as follows.
Group 1: Control vehicle containing 2% Solutol HS15, 4.5% (w/v) mannitol, and 25 mM (0.38% w/v) histidine in water, pH 6.0 (2% Solutol vehicle)
Group 2: 15 mg/mL Cinacalcet HCl in 20% Captisol in water, pH 7.0
Group 3: 5 mg/mL Cinacalcet HCl in 20% Captisol in water, pH 7.0
Group 4: 0.285 mg/mL BIM-44002 in saline
Groups 5 and 6: 0.15 mg/mL SP67 in 2% Solutol vehicle
   The dose volumes whereas listed in the above table.

### Route of Administration and Dosing Procedure

Each animal was administered a single IV slow push or PO gavage dose. The IV dose was administered via the FVC. Doses are as summarized above.

### Sample Collections

Blood samples were collected from each animal and processed to serum.

### Sample Times for Groups 1-6:

pre-dose, 1, 2, 4, 8, 12, 24, 48 h post-dose, and optionally 80 and 144 h post-dose (Only if the serum calcium concentrations at 48 h are greater than pre-dose concentrations). Animals were not euthanized until the 48 h serum calcium concentration data indicate similarity to pre-dose concentrations, or at 144 h.

### Blood Volume:

For the 1 h sample only, the volume was 0.5 mL. All other sample volumes were 0.3 mL. If applicable, the 144 h sample was a terminal sample of as much volume as possible.

### Anticoagulant:

None (serum separator tubes); an additional K₂EDTA tube for each 1 h sample

### Blood Collection:

Non-terminal blood samples were collected from the JVC. There was no blood replacement, but there was a flush of the cannula with heparinized saline. If the cannula failed, retroorbital sinus or tail bleeding was used within QPS IACUC guidelines. If applicable, the 144 h sample was a terminal sample taken by cardiac puncture.

### Blood Processing:

The 1 h sample only was split with 0.3 mL placed into a serum separator tube and processed to serum, and 0.2 mL processed to plasma. Blood collection tubes for plasma were placed on ice until processing. The 1 h plasma samples were stored at −70° C. until needed for possible concentration analysis. Blood for serum were allowed to clot at room temperature and then centrifuged to collect serum. Serum was transferred to labeled cryovials and immediately frozen on dry ice. All serum specimens were stored at −70° C. until delivery to Antech for serum calcium determination.

### Sample Analysis and Data Analysis

### Concentrations

Plasma concentrations at 1 h were determined only if needed after the serum calcium results are available. If performed, samples were analyzed for test article concentrations at using an LC/MS/MS method, according to the criteria listed below.
Quantitation by LC/MS/MS with internal standard
Anticipated assay range is 3-3000 nM
Calibration curve before and after sample analysis (N=2) prepared in blank matrix will consist of the following standards
Double blank
Single Blank (Internal Standard only)
Minimum of 6 standard concentration
Acceptance criteria: Five (5) standard concentrations minimum within the curve, must contain at least one (1) standard at both bottom and top of the range back calculated to ±20% of their nominal concentrations. It is acceptable to remove the upper or lower standards to bring the curve into ±20% nominal.

### Serum Calcium

Serum specimens were analyzed for serum calcium by a standard Beckman colorimetric assay performed by Antech Diagnostics, Lake Success, N.Y.

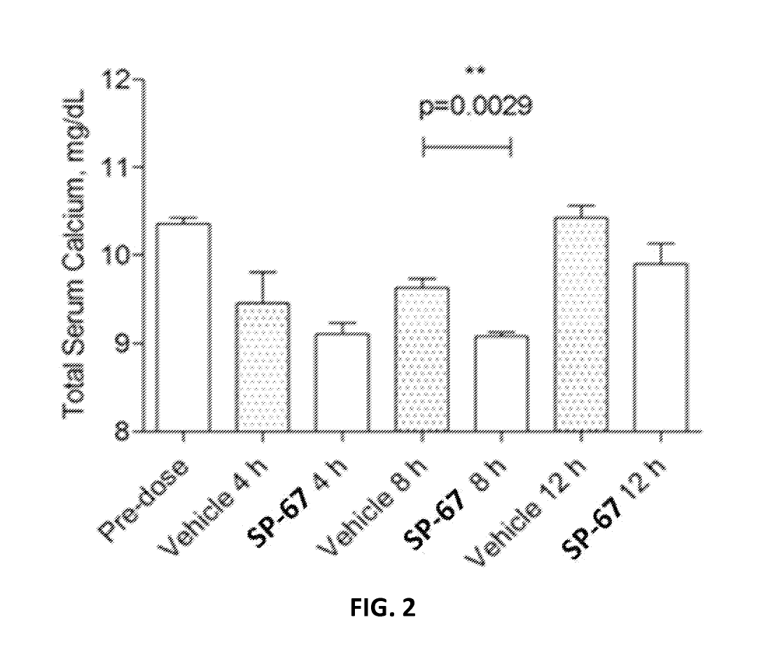

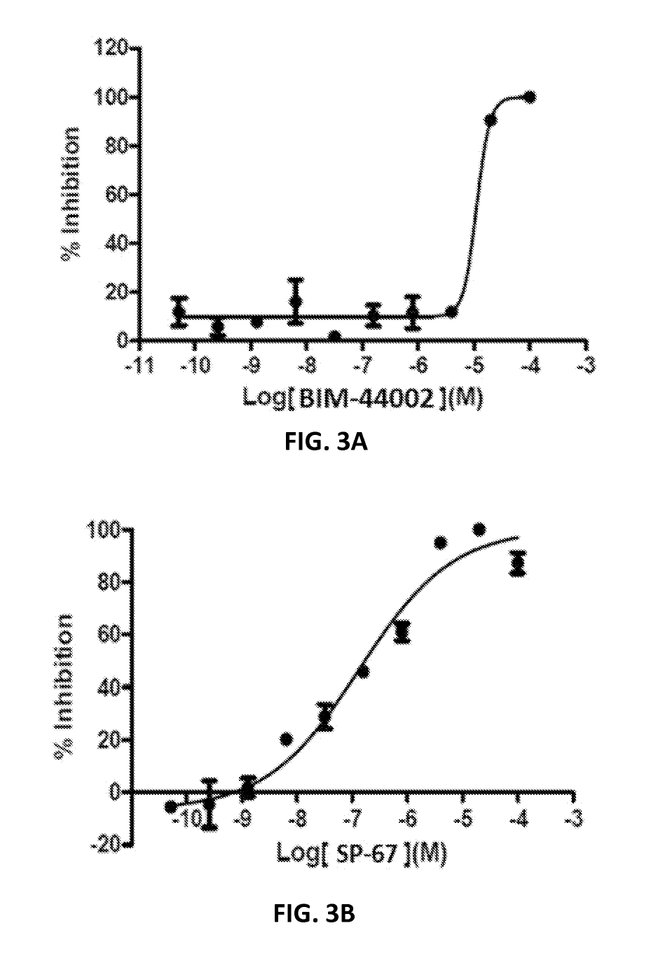

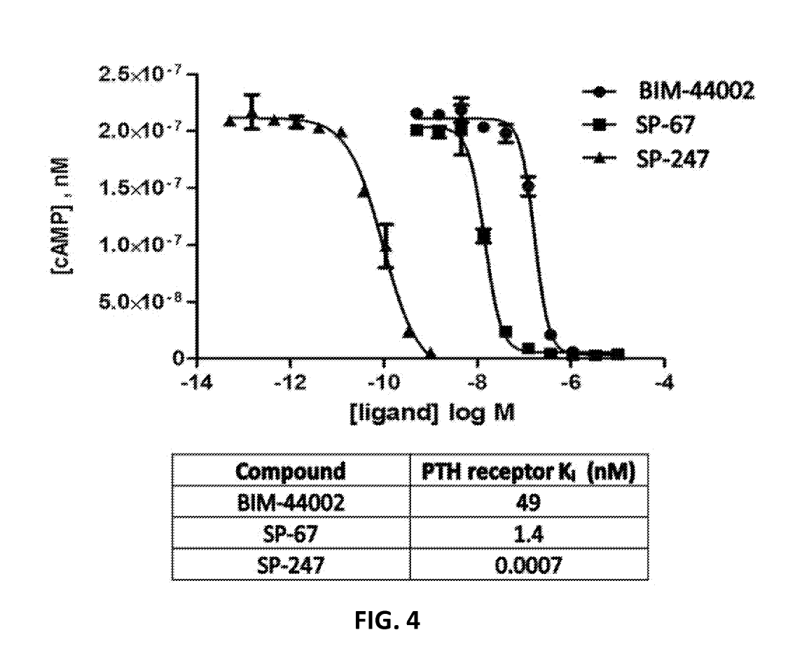

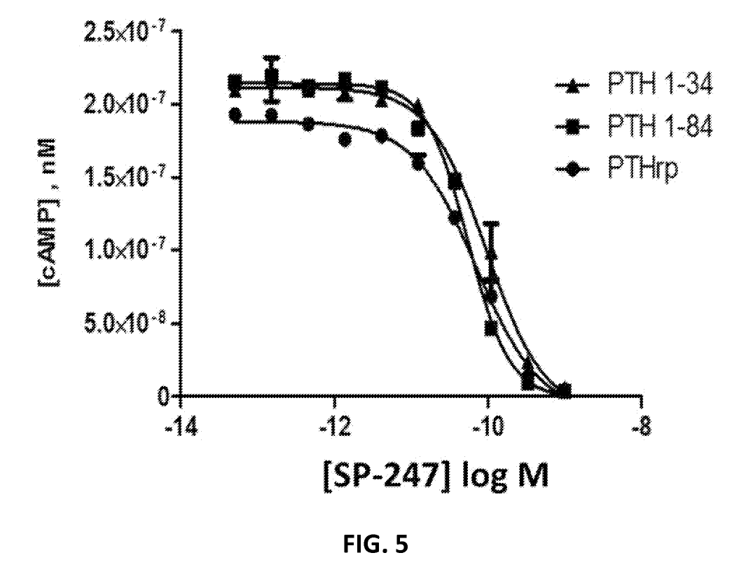

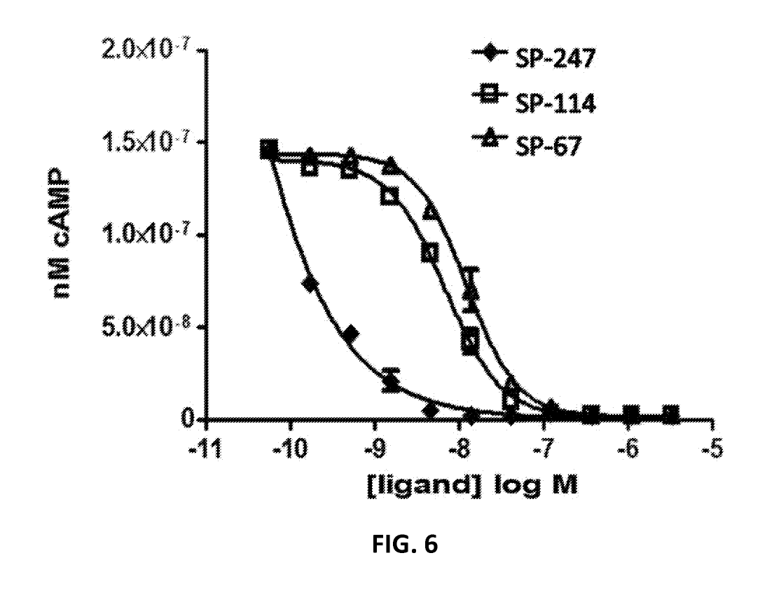

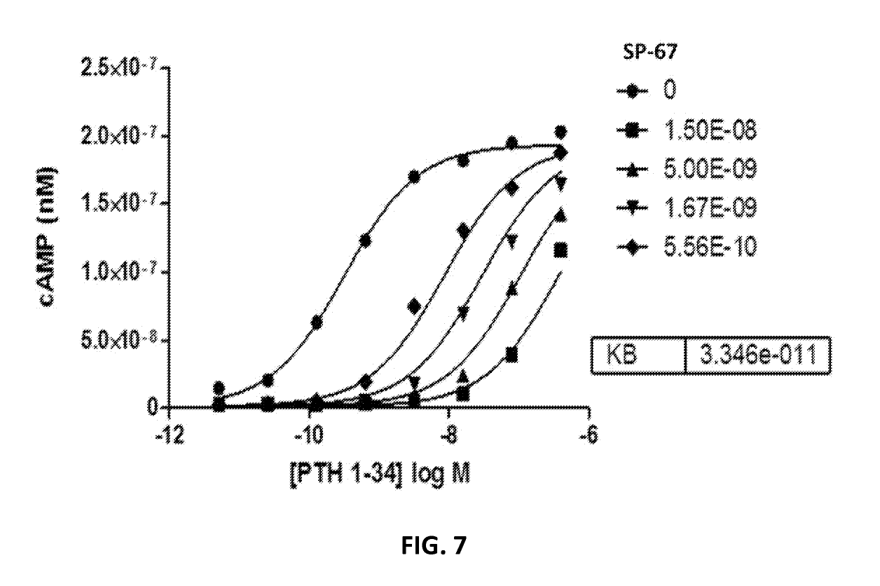

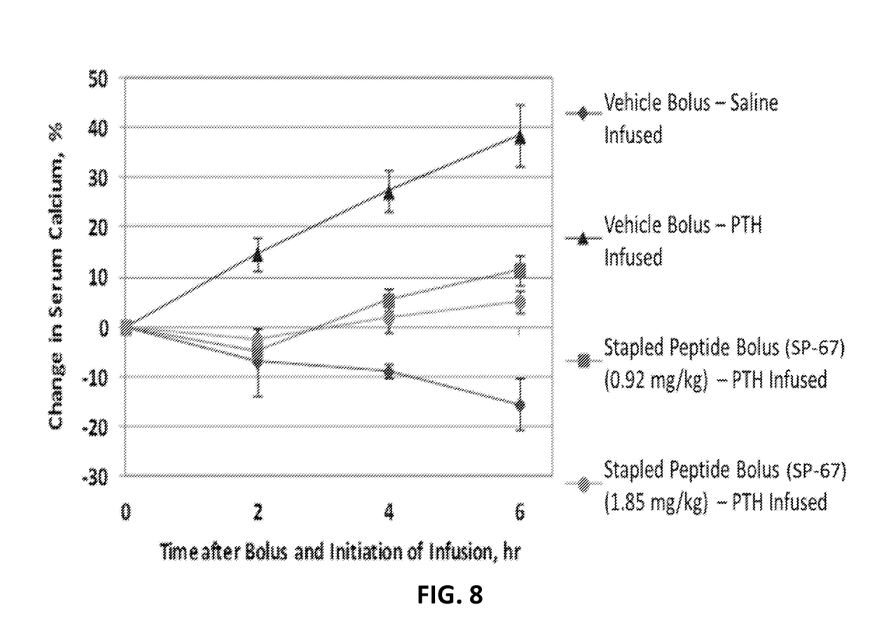

Serum calcium values are shown in FIG. 2.

### Example 8

### Determination of K_{B} by Schild Analysis in SaOS-2 Cells

A competitive inhibitor of PTHR1 competes for agonist (PTH[1-34]) binding to a receptor, and shifts the agonist dose-response curve to the right without changing the maximum response. By fitting all the curves globally, the affinity of the competitive inhibitor for the receptor can be determined. SaOS-2 cells were prepared in assay buffer and dispensed into plates as for IC₅₀ determination. A PTH[1-34] dose response was determined in the presence of increasing concentrations of antagonist (0, 1, 3, 10, 30 nM). The dose-response curves were fit with 3-parameter non-linear equations to determine EC₅₀ at each antagonist concentration and K_{B}was determined using GraphPad Prism (Gaddum/Schild EC₅₀ Shift Equation). The following legend is used in Table 24: <1 nM (“++++”), 1-5 nM (“+++”), 6-20 nM (“++”), >20 nM (“+”).

TABLE 24

| SP# | K_{B} (nM) | SP# | K_{B} (nM) | SP# | K_{B} (nM) |
|---|---|---|---|---|---|
| LP74 | +++ | 200 | ++++ | 232 | ++++ |
| 239 | ++++ | 201 | +++ | 245 | ++ |
| 161 | ++++ | 204 | +++ | 246 | +++ |
| 162 | ++++ | 218 | ++++ | 247 | ++++ |
| 164 | + | 220 | ++++ | 257 | ++++ |
| 173 | ++++ | 221 | ++++ | 259 | ++++ |
| 182 | ++++ | 226 | +++ | 261 | ++++ |
| 183 | +++ | 228 | +++ | 262 | ++++ |
| 188 | ++++ | | | | |

### Example 9

### Potency Assay in SaOS-2 Using PTH (1-84) and PTHrP (1-34)

Assays were performed as for PTH[1-34] activity assays except human PTH[1-84] or PTHrP[1-34] purchased from Bachem (Cat no. H-1370 or H-6630) was used as ligand. For example, the peptidomimetic macrocycles SP247, SP226, SP228, SP232, SP245, and SP246, were found to have an IC₅₀ of <0.5 nM or <10 nM.

### Example 10

### PTH Antagonist in a TPTx Rat Model of PTH (1-34) Induced Hypercalcemia

Experiments were conducted assess the effect of a PTH antagonist SP#63 in a thyroparathyroidectomized (TPTx) rat model of PTH (1-34) induced hypercalcemia. Thyroid hormone was given as supportive therapy prior to start of PTH infusion.

SP#63 or the vehicle for SP#63 was administered as an intravenous (IV) bolus in PTH infused thyroparathyroidectomized (TPTx) Sprague-Dawley rats. SP#63 was administered at 0.925 mg/kg and 1.850 mg/kg, 1 to 3 min before the initiation of the IV infusion. PTH (administered at 1.25 μg/kg/h) or the vehicle for PTH were infused via a femoral catheter over a period of 6 hours at a rate of 1 mL/kg/h. Total and ionized calcium were measured from blood samples collected at 0, 2, 4 and 6 hours during the IV infusion. A summary of the experimental design and the in-life procedures and analytical endpoints are summarized in Tables 25 and 26.

TABLE 25
Experimental Design

| | | | | | | | Dose | |
|---|---|---|---|---|---|---|---|---|
| | | Route/ | Dose | | Route/ | Dose | Vol. | # |
| Grp | Treat 1 | Dose Vol. | mg/kg | Treat 2 | Frequency* | mg/kg | mL/kg | Animals |
| 1 | Vehicle | IV, 1 mL/kg/h | — | Vehicle for | IV bolus, QD | — | 0.66 | 5 |
| | for PTH | (6 h) | | SP# 63 | (before infuse start) | | | |
| | infusion | | | | | | | |
| 2 | PTH | IV, 1 mL/kg/h | 1.25 | Vehicle for | IV bolus, QD | — | 0.66 | 5 |
| | infusion | (6 h) | μg/kg/h | SP# 63 | (before infuse start) | | | |
| 3 | PTH | IV, 1 mL/kg/h | 1.25 | SP# 63 | IV bolus, QD | 0.925 | 0.66 | 5 |
| | infusion | (6 h) | μg/kg/h | | (before infuse start) | | | |
| 4 | PTH | IV, 1 mL/kg/h | 1.25 | SP# 63 | IV bolus, QD | 1.850 | 0.66 | 5 |
| | infusion | (6 h) | μg/kg/h | | (before infuse start) | | | |

*IV = intravenous; QD = once a day (before infusion start)

TABLE 26
In-life procedures and analytical endpoints
In-life procedures

| | |
|---|---|
| Body weight (BW) | Once during the acclimation period, on Day −2 (BW randomization) prior to |
| | surgery and prior to treatment initiation. |
| Baseline calcium | All animals-48 h before initiation of treatment in Group 1. |
| levels (Day −2 in | Animals were fasted overnight. |
| the morning) | Blood samples (0.7 ml) were collected in the morning from the jugular vein, |
| | for total serum calcium (in serum separator tubes). |
| Femoral | All animals-48 h before initiation of treatment. Animals were continuously |
| catheterization | infused with 0.9% Sodium Chloride Injection, USP, at a rate of 0.4 mL/h |
| (Day −2 in the | until initiation of treatment. |
| afternoon) | |
| Dosing | On the day of treatment animals received an intravenous (IV) bolus |
| | (0.66 mL/kg) of the test item or vehicle for the test item, followed by a 6 h |
| | infusion of saline (Group 1) or PTH (Groups 2, 3 and 4) at a 1 mL/kg/h rate. |
| Terminal | At the end of the infusion period all animals were euthanized by |
| procedures | exsanguination from the abdominal aorta (performed under isoflurane |
| | anaesthesia) and discarded without examination. |

Analytical endpoints

| | |
|---|---|
| Blood collection | Blood was collected at 0, 2, 4 and 6 hours post start of infusion, for total |
| | serum calcium (0.7 mL in serum separator tubes) and ionized calcium |
| | (0.3 mL whole blood collected in heparinized syringes). |

Calcium levels in the vehicle treated animals were not significantly different over the course of the IV infusion compared to the 0 h time point (small decrease observed over time probably caused by the prolonged fasting period) while the PTH infusion in hypocalcemic TPTx rats caused an increase in blood calcium levels that reached physiological values at 4 and 6 h after IV infusion was initiated (4.99 mg/dL at 6 h compared to 3.36 mg/dL at 0 h for the ionized calcium, and 9.92 mg/dL at 6 h compared to 7.22 mg/dL at 0 h for the total calcium), thereby confirming that the model was suitable for assessing test article effects on the PTH induced hypercalcemia in TPTx rats.

A strong inhibition of the PTH induced hypercalcemia was observed at both doses of SP#63 consistent with the effects of a PTH antagonist. In the 0.925 mg/kg SP#63 treated animals, the ionized calcium levels increased to 3.95 mg/dL at 6 h (compared to 3.01 mg/dL in vehicle treated animals and 4.99 mg/dL in the PTH treated rats), equivalent to a 53% inhibition of the PTH response. Similarly, the total calcium levels increased to 7.48 mg/dL in the same group (compared to 5.73 mg/dL in the vehicle treated group and 9.92 mg/dL in the PTH treated group), equivalent to a 58% inhibition of the PTH response. The PTH inhibition in the 1.850 mg/kg SP#63 treated animals was similar to the inhibition observed in the SP#63 animals treated at a lower dose. Ionized calcium increased to 3.80 mg/dL, equivalent to a 60% inhibition of the PTH response, while the total calcium increased to 7.45 mg/dL, a 59% inhibition of the PTH response.

Shortly after the IV bolus administration of SP#63, ataxia/lethargy and generalized edema was observed in most of the treated animals, especially in the animals treated at the highest. Ataxia/lethargy resolved soon (within 30 minutes) after the IV bolus administration while the edema started to diminish towards the end of the 6 h infusion period.

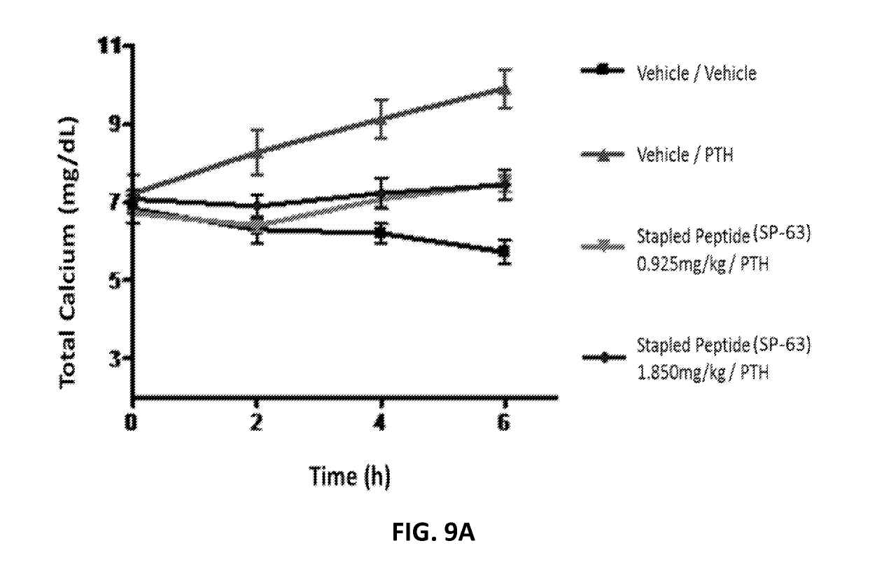

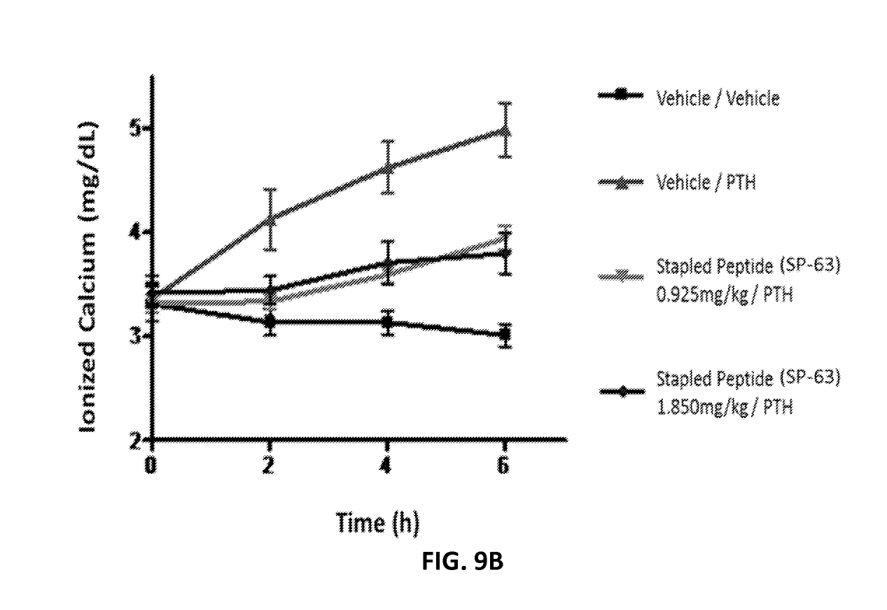

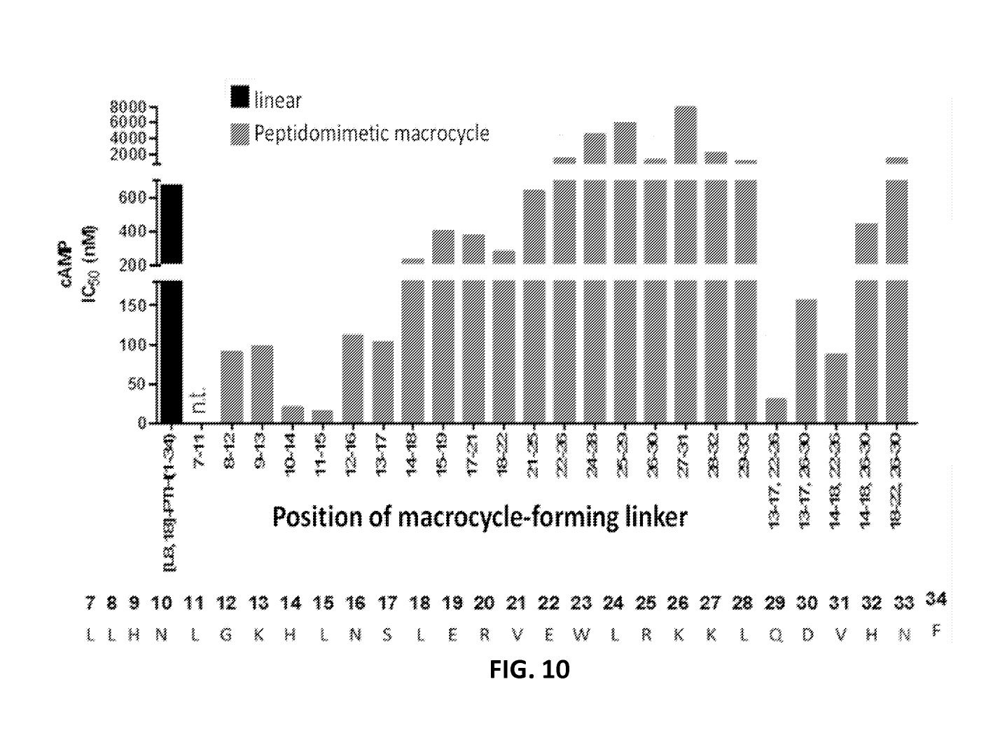

The ionized and total calcium values are presented in Table 27 and FIGS. 9A and 9B.

TABLE 27
Ionized and total blood calcium results

| | Group 1 Vehicle/Vehicle | | | | | | Group 2 Vehicle/PTH | | | | | |
|---|---|---|---|---|---|---|---|---|---|---|---|---|
| Time | Ionized calcium | | | | | | Ionized calcium | | | | | |
| point | 1001¹ | 1002 | 1003 | 1004 | 1005 | Avg. | 2001 | 2002 | 2003 | 2004 | 2005 | Avg. |
| 0 | | 3.57 | 2.96 | 3.62 | 3.1 | 3.31 | 4.01 | clotted | 3.16 | 3.21 | 3.07 | 3.36 |
| 2 h | | 3.43 | 2.86 | 3.26 | 3 | 3.14 | 4.85 | 4.68 | 3.75 | 4.08 | 3.27 | 4.13 |
| 4 h | | 3.3 | 2.93 | 3.36 | 2.94 | 3.13 | 5.29 | 5.02 | 4.34 | 4.58 | 3.88 | 4.62 |
| 6 h | | 3.25 | 2.86 | 3.13 | 2.81 | 3.01 | 5.71 | 5.23 | 4.79 | 5.06 | 4.15 | 4.99 |
| Time | Total calcium | | | | | | Total calcium | | | | | |
| point | 1001 | 1002 | 1003 | 1004 | 1005 | Avg. | 2001 | 2002 | 2003 | 2004 | 2005 | Avg. |
| 0 | | 7.2 | 6.1 | 6.4 | 7.6 | 6.83 | 8.4 | 8.3 | 6.3 | 6.7 | 6.4 | 7.22 |
| 2 h | | 6.9 | 5.7 | 6.9 | 5.7 | 6.30 | 9.8 | 9.3 | 7.5 | 8.2 | 6.6 | 8.28 |
| 4 h | | 6.6 | 5.6 | 6 | 6.6 | 6.20 | 10.5 | 9.8 | 8.7 | 9.1 | 7.6 | 9.14 |
| 6 h | | 6.4 | 5.2 | 6 | 5.3 | 5.73 | 11.4 | 10.1 | 9.7 | 10.1 | 8.3 | 9.92 |
| | Baseline calcium levels (Day −2) | | | | | | Baseline calcium levels (Day −2) | | | | | |
| | 6.6 | 7.8 | 8.7 | 8.7 | 7.6 | 8.20 | 6.2 | 7.6 | 8.4 | 9.1 | 7.8 | 8.23 |

| | Group 3 SP#63 (0.925 mpk)/PTH | | | | | | Group 4 SP#63 (1.850 mpk)/PTH | | | | | |
|---|---|---|---|---|---|---|---|---|---|---|---|---|
| Time | Ionized calcium | | | | | | Ionized calcium | | | | | |
| point | 3001 | 3002 | 3003 | 3004 | 3005 | Avg. | 4001 | 4002² | 4003 | 4004 | 4005 | Avg. |
| 0 | 3.39 | 3.4 | 3.51 | 3.06 | 3.21 | 3.31 | 3.28 | 4.26 | 3.7 | 3.45 | 3.23 | 3.42 |
| 2 h | 3.42 | 3.27 | 3.59 | 3.13 | 3.27 | 3.34 | 3.14 | 4.18 | 3.7 | 3.62 | 3.3 | 3.44 |
| 4 h | clotted | 3.61 | 3.78 | 3.36 | 3.62 | 3.59 | clotted | clotted | 3.97 | 3.84 | 3.31 | 3.71 |
| 6 h | 4.32 | 3.84 | 4 | 3.61 | 3.96 | 3.95 | 3.52 | clotted | 4.3 | 3.92 | 3.45 | 3.80 |
| Time | Total calcium | | | | | | Total calcium | | | | | |
| point | 3001 | 3002 | 3003 | 3004 | 3005 | Avg. | 4001 | 4002² | 4003 | 4004 | 4005 | Avg. |
| 0 | 6.9 | 6.8 | 7.1 | 6.3 | 6.5 | 6.72 | 6.7 | 9 | 7.7 | 7.1 | 6.8 | 7.08 |
| 2 h | 6.6 | 6 | 6.9 | 6.1 | 6.4 | 6.40 | 6.2 | 8.7 | 7.5 | 7.3 | 6.6 | 6.90 |
| 4 h | 7.6 | 6.8 | 7.3 | 6.5 | 7.2 | 7.08 | 6.6 | 8.7 | 8.1 | 7.7 | 6.5 | 7.23 |
| 6 h | 8.2 | 7.2 | 7.4 | 6.9 | 7.7 | 7.48 | 6.9 | 8.6 | 8.4 | 7.7 | 6.8 | 7.45 |
| | Baseline calcium levels (Day −2) | | | | | | Baseline calcium levels (Day −2) | | | | | |
| | 6.4 | 8.4 | 9.1 | 7 | 8.7 | 8.30 | 7.2 | 6.1 | 8.7 | 8.7 | 8.2 | 7.93 |

¹Animal excluded (catheter malfunction)
²Animal excluded (high baseline values, clotted samples)

Parathyroid hormone (PTH) infusion in thyroparathyroidectomized (TPTx) rats resulted in a significant increase in total and ionized blood calcium levels at 4 and 6 hours after the start of infusion compared to the vehicle treated animals, confirming that the model was suitable for assessing the effects of PTH antagonists. Treatment with SP#63 at 0.925 and 1.850 mg/kg resulted in a strong inhibition of PTH induced hypercalcemia (ranging between 53% and 60%) at both tested doses. Total and ionized blood calcium levels for SP#63-treated groups were significantly lower than for animals treated with PTH(1-34).

### Example 11

### In Vitro Pharmacology: Cellular and Nuclear Receptor Functional Assays

Control Response Assay:

Cells were prepared in assay buffer and dispensed into plates as in Example 8. Cells were treated with a fixed concentration of the indicated ligands in the absence of antagonist. After 10-30 minutes, cAMP concentrations were determined for each of the ligand stimulations using an HTRF based kit (control response).

Agonist and Antagonist Assay:

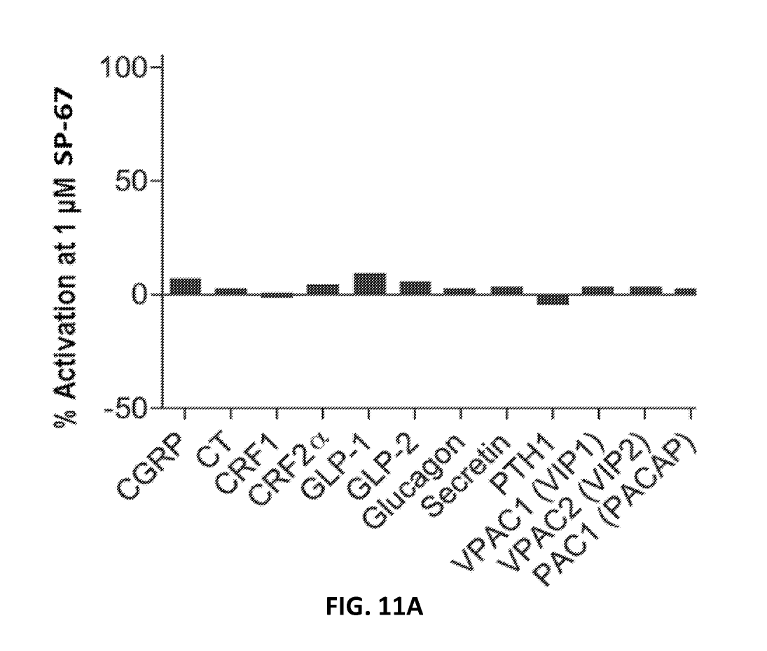

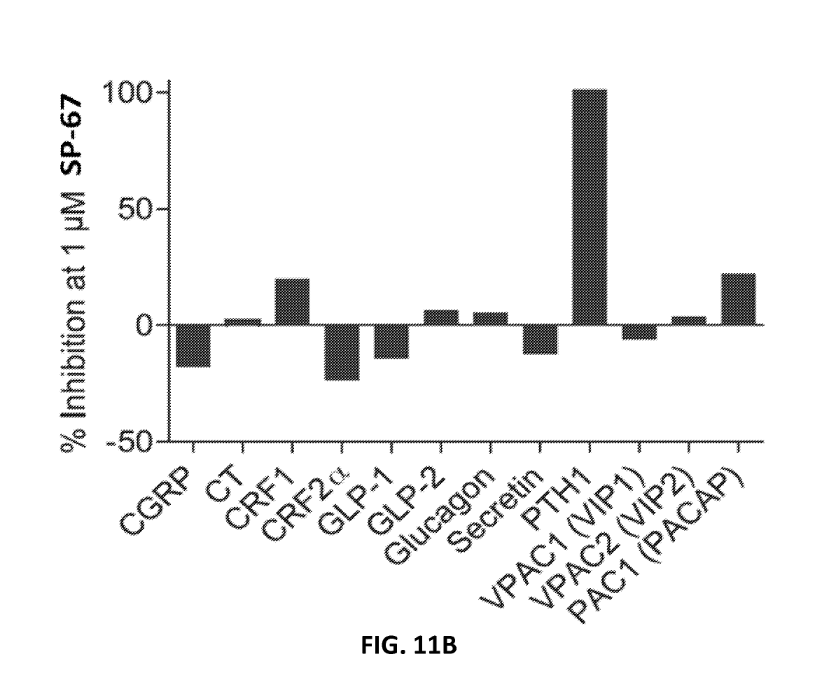

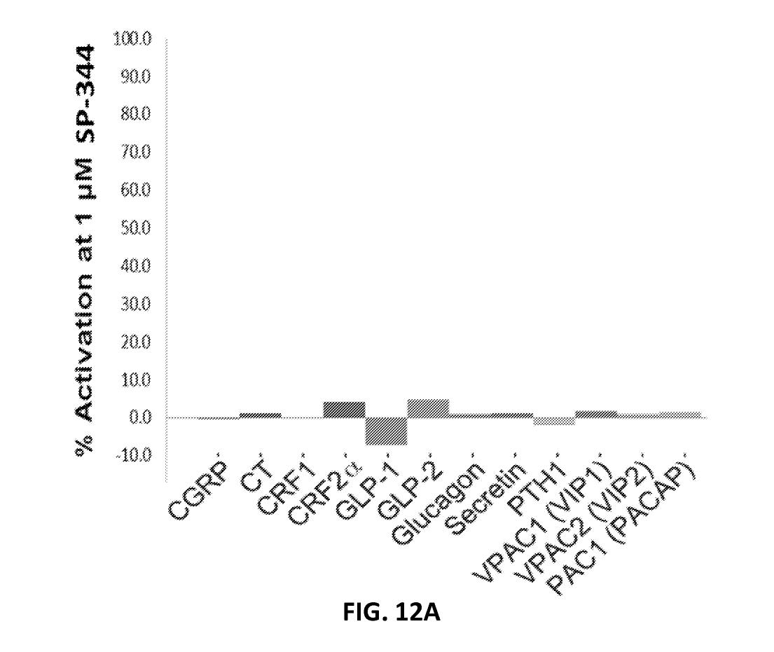

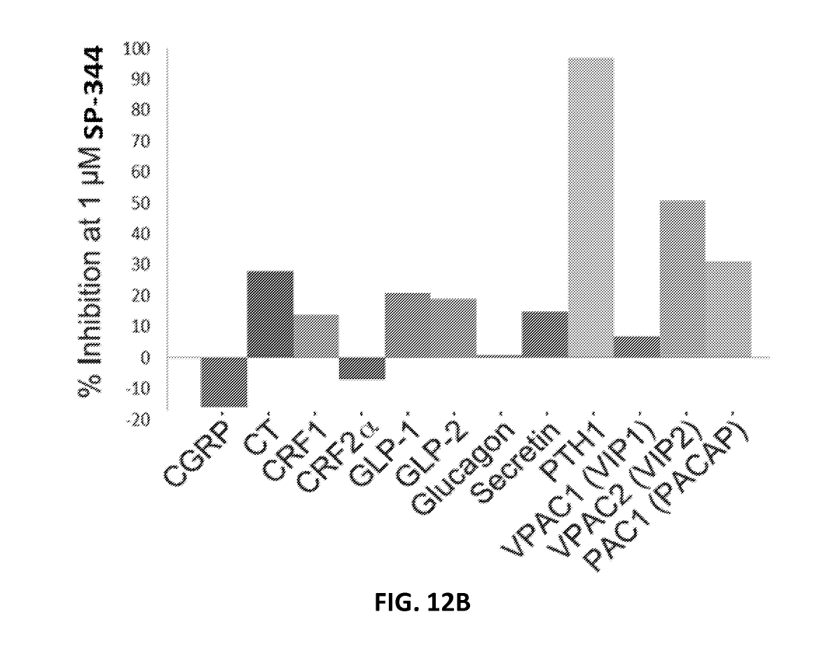

Cells were prepared in assay buffer and dispensed into plates as above. SP-67 or SP-344 were diluted in assay buffer and added to the cells at a final concentration of 1 μM Following a 10 minute incubation (room temperature), the indicated ligand was added to the cells at a fixed concentration to stimulate cAMP production. After 10-30 minutes at room temperature or 37° C., cAMP concentrations were determined for each well using an HTRF based kit (test response). The test response results in FIGS. 11A and 12A are expressed as a percent of control agonist response ((test response/control response)*100). The results in FIGS. 11B and 12B are expressed as a percent inhibition of the respective control response (100−(test response/control response)*100). Experimental conditions can be seen in Table 28:

TABLE 28

| Assay | Source | Stimulation | Incubation |
|---|---|---|---|
| CGRP (agonist) | Human recombinant (CHO | None (10 nM | 30 min RT |
| | cells) | hCRGPα for control) | |
| CGRP (antagonist) | Human recombinant (CHO | (0.3 nM hCRGPα) | 30 min RT |
| | cells) | | |
| CT (Calcitonin) (agonist) | T47 cells (endogenous) | None (1 μM human | 10 min RT |
| | | calcitonin for | |
| | | control) | |
| CT (Calcitonin) (antagonist) | T47 cells (endogenous) | (30 nM human | 10 min RT |
| | | calcitonin) | |
| CRF₁ (agonist) | Human recombinant (CHO | None (1 μM ovine | 30 min 37° C. |
| | cells) | CRF for control) | |
| CRF₁ (antagonist) | Human recombinant (CHO | 30 nM ovine CRF | 30 min 37° C. |
| | cells) | | |
| CRF_{2α} (agonist) | Human recombinant | None (1 μM human | 30 min RT |
| | (HEK293 cells) | CRF for control) | |
| CRF_{2α} (antagonist) | Human recombinant | 100 nM ovine CRF | 30 min RT |
| | (HEK293 cells) | | |
| GLP-1 (agonist) | βTC5 cells (endogenous) | None (10 nM GLP- | 10 min RT |
| | | 1(7-37) for control) | |
| GLP-1 (antagonist) | βTC5 cells (endogenous) | 0.3 nM GLP-1(7-37) | 10 min RT |
| GLP-2 (agonist) | Human recombinant (CHO | None (10 nM GLP- | 30 min RT |
| | cells) | 2(1-34) for control) | |
| GLP-2 (antagonist) | Human recombinant (CHO | 0.1 nM GLP-2(1-34) | 30 min RT |
| | cells) | | |
| Glucagon (agonist) | Human recombinant (CHO | None (100 nM | 10 min 37° C. |
| | cells) | glucagon for control) | |
| Glucagon (antagonist) | Human recombinant (CHO | 1 nM glucagon | 10 min 37° C. |
| | cells) | | |
| secretin (agonist) | Human recombinant (CHO | None (300 nM | 30 min RT |
| | cells) | human secretin for | |
| | | control) | |
| secretin (antagonist) | Human recombinant (CHO | 0.3 nM human | 30 min RT |
| | cells) | secretin | |
| PTH1 (agonist) | SaOS2 cells (endogenous) | None (1 μM | 10 min RT |
| | | PTHrP(1-34) for | |
| | | control) | |
| PTH1 (antagonist) | SaOS2 cells (endogenous) | 3 nM PTHrP(1-34) | 10 min RT |
| Pac₁ (PACAP) (agonist) | Human recombinant (CHO | None (1 nM | 30 min RT |
| | cells) | PACAP₁₋₃₈ for | |
| | | control) | |
| Pac₁ (PACAP) (antagonist) | Human recombinant (CHO | 0.1 nM PACAP₁₋₃₈ | 30 min RT |
| | cells) | | |
| VPAC₁ (VIP₁) (agonist) | HT-29 cells (endogenous) | None (100 nM VIP | 10 min 37° C. |
| | | for control) | |
| VPAC₁ (VIP₁) | HT-29 cells (endogenous) | 3 nM VIP | 10 min 37° C. |
| (antagonist) | | | |
| VPAC₂ VIP₂) (agonist) | Human recombinant (CHO | None (100 nM VIP | 30 min RT |
| | cells) | for control) | |
| VPAC₂(VIP₂) | Human recombinant (CHO | 3 nM VIP | 30 min RT |
| (antagonist) | cells) | | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A peptidomimetic macrocycle comprising at least one macrocycle-forming linker and an amino acid sequence with at least about 60%, 65%, 70%, 75%, 80%, 85%, 90%, 95%, 96%, 97%, 98%, 99% or 100% sequence identity to a sequence of Table 1a, 1b, 2a, or 2b, wherein the peptidomimetic macrocycle comprises at least two non-natural amino acids connected by a first macrocycle-forming linker of the at least one macrocycle-forming linker.

2. The peptidomimetic macrocycle of claim 1, wherein the first macrocycle-forming linker connects amino acids 7 and 11, 7 and 14, 8 and 12, 9 and 13, 10 and 14, 11 and 15, 12 and 16, 13 and 17, 14 and 18, 14 and 21, 15 and 19, 15 and 22, 17 and 24, 18 and 22, 18 and 25, 22 and 26, 22 and 29, 24 and 28, 25 and 32, 26 and 30, 26 and 33, or 27 and 31.

3. The peptidomimetic macrocycle of claim 2, wherein the first macrocycle-forming linker connects amino acids 7 and 11, 8 and 12, 9 and 13, 10 and 14, 13 and 17, 14 and 18, or 18 and 22.

4. The peptidomimetic macrocycle of claim 2 or 3, wherein the first macrocycle-forming linker connects amino acids 9 and 13.

5. The peptidomimetic macrocycle of claim 2 or 3, wherein the first macrocycle-forming linker connects amino acids 10 and 14 or 11 and 15.

6. A peptidomimetic macrocycle comprising at least one macrocycle-forming linker and an amino acid sequence with at least about 60%, 65%, 70%, 75%, 80%, 85%, 90%, 95%, 96%, 97%, 98%, 99% or 100% sequence identity to a sequence of Table 1a, wherein the peptidomimetic macrocycle comprises at least two non-natural amino acids connected by a first macrocycle-forming linker of the at least one macrocycle-forming linker, wherein the first macrocycle-forming linker connects amino acids 10 and 14 or 11 and 15.

7. The peptidomimetic macrocycle of claim 2 or 3, wherein the first macrocycle-forming linker connects amino acids 13 and 17.

8. The peptidomimetic macrocycle of claim 2 or 3, wherein the first macrocycle-forming linker connects amino acids 14 and 18.

9. The peptidomimetic macrocycle of claim 2 or 3, wherein the first macrocycle-forming linker connects amino acids 18 and 22.

10. The peptidomimetic macrocycle of claim 2, wherein the first macrocycle-forming linker connects amino acids 24 and 28 or 27 and 31.

11. A peptidomimetic macrocycle comprising at least one macrocycle-forming linker and an amino acid sequence with at least about 60%, 65%, 70%, 75%, 80%, 85%, 90%, 95%, 96%, 97%, 98%, 99% or 100% sequence identity to a sequence of Table 1a, wherein the peptidomimetic macrocycle comprises at least two non-natural amino acids connected by a first macrocycle-forming linker of the at least one macrocycle-forming linker, wherein the first macrocycle-forming linker connects amino acids 24 and 28 or 27 and 31.

12. The peptidomimetic macrocycle of any one of claims 2-11, wherein the at least one macrocycle-forming linker comprises a second macrocycle-forming linker.

13. The peptidomimetic macrocycle of claim 12, wherein the second macrocycle-forming linker connects amino acids 18 and 22, 22 and 26, 24 and 28, or 26 and 30.

14. The peptidomimetic macrocycle of claim 12 or 13, wherein the second macrocycle-forming linker connects amino acids 22 and 26.

15. The peptidomimetic macrocycle of claim 12 or 13, wherein the second macrocycle-forming linker connects amino acids 24 and 28.

16. The peptidomimetic macrocycle of claim 12 or 13, wherein the second macrocycle-forming linker connects amino acids 26 and 30.

17. The peptidomimetic macrocycle of claim 12 or 13, wherein the second macrocycle-forming linker connects amino acids 18 and 22 or 24 and 28.

18. The peptidomimetic macrocycle of claim 12 or 13, wherein a first macrocycle-forming linker connects amino acids 8 and 12, and the second macrocycle-forming linker connects amino acids 22 and 26.

19. The peptidomimetic macrocycle of claim 12 or 13, wherein the first macrocycle-forming linker connects amino acids 13 and 17, and the second macrocycle-forming linker connects amino acids 22 and 26.

20. The peptidomimetic macrocycle of claim 12 or 13, wherein the first macrocycle-forming linker connects amino acids 13 and 17, and the second macrocycle-forming linker connects amino acids 24 and 28.

21. The peptidomimetic macrocycle of claim 12 or 13, wherein the first macrocycle-forming linker connects amino acids 14 and 18, and the second macrocycle-forming linker connects amino acids 22 and 26.

22. The peptidomimetic macrocycle of claim 12 or 13, wherein a first macrocycle-forming linker connects amino acids 7 and 11, and the second macrocycle-forming linker connects amino acids 22 and 26.

23. The peptidomimetic macrocycle of any one of claims 12-22, wherein the at least one macrocycle-forming linker comprises a third macrocycle-forming linker.

24. The peptidomimetic macrocycle of claim 23, wherein the third macrocycle-forming linker connects amino acids 27-31.

25. The peptidomimetic macrocycle of any one of claims 1-24, wherein the peptidomimetic macrocycle has an amino acid sequence with at least about 60%, 65%, 70%, 75%, 80%, 85%, 90%, 95%, 96%, 97%, 98%, 99% or 100% sequence identity to a sequence of Table 7.

26. The peptidomimetic macrocycle of claim 25, wherein the peptidomimetic macrocycle has an amino acid sequence with at least about 75% sequence identity to a sequence of Table 7.

27. The peptidomimetic macrocycle of claim 25, wherein the peptidomimetic macrocycle has an amino acid sequence with at least about 90% sequence identity to a sequence of Table 7.

28. The peptidomimetic macrocycle of claim 25, wherein the peptidomimetic macrocycle has an amino acid sequence with at least about 95% sequence identity to a sequence of Table 7.

29. The peptidomimetic macrocycle of claim 25, wherein the peptidomimetic macrocycle has 100% sequence identity to a sequence of Table 7.

30. The peptidomimetic macrocycle of any one of claims 1-24, wherein the peptidomimetic macrocycle an amino acid sequence with at least about 60%, 65%, 70%, 75%, 80%, 85%, 90%, 95%, 96%, 97%, 98%, 99% or 100% sequence identity to a sequence of Table 3b.

31. The peptidomimetic macrocycle of claim 30, wherein the peptidomimetic macrocycle has an amino acid sequence with at least about 75% sequence identity to a sequence of Table 3b.

32. The peptidomimetic macrocycle of claim 30, wherein the peptidomimetic macrocycle has an amino acid sequence with at least about 90% sequence identity to a sequence of Table 3b.

33. The peptidomimetic macrocycle of claim 30, wherein the peptidomimetic macrocycle has an amino acid sequence with at least about 95% sequence identity to a sequence of Table 3b.

34. The peptidomimetic macrocycle of claim 30, wherein the peptidomimetic macrocycle has an amino acid sequence with 100% sequence identity to a sequence of Table 3b.

35. The peptidomimetic macrocycle of any one of claims 1-24, wherein the peptidomimetic macrocycle has an amino acid sequence with at least about 60%, 65%, 70%, 75%, 80%, 85%, 90%, 95%, 96%, 97%, 98%, 99% or 100% sequence identity to a sequence of Table 6.

36. The peptidomimetic macrocycle of claim 35, wherein the peptidomimetic macrocycle has an amino acid sequence with at least about 75% sequence identity to a sequence of Table 6.

37. The peptidomimetic macrocycle of claim 35, wherein the peptidomimetic macrocycle has an amino acid sequence with at least about 90% sequence identity to a sequence of Table 6.

38. The peptidomimetic macrocycle of claim 35, wherein the peptidomimetic macrocycle has an amino acid sequence with at least about 95% sequence identity to a sequence of Table 6.

39. The peptidomimetic macrocycle of claim 35, wherein the peptidomimetic macrocycle has an amino acid sequence with 100% sequence identity to a sequence of Table 6.

40. The peptidomimetic macrocycle of any one of claims 1-24, wherein the peptidomimetic macrocycle has an amino acid sequence with at least about 60%, 65%, 70%, 75%, 80%, 85%, 90%, 95%, 96%, 97%, 98%, 99% or 100% sequence identity to a sequence of Table 8.

41. The peptidomimetic macrocycle of claim 40, wherein the peptidomimetic macrocycle has an amino acid sequence with at least about 75% sequence identity to a sequence of Table 8.

42. The peptidomimetic macrocycle of claim 40, wherein the peptidomimetic macrocycle has an amino acid sequence with at least about 90% sequence identity to a sequence of Table 8.

43. The peptidomimetic macrocycle of claim 40, wherein the peptidomimetic macrocycle has an amino acid sequence with at least about 95% sequence identity to a sequence of Table 8.

44. The peptidomimetic macrocycle of claim 40, wherein the peptidomimetic macrocycle has an amino acid sequence with 100% sequence identity to a sequence of Table 8.

45. The peptidomimetic macrocycle of claim 40, wherein the peptidomimetic macrocycle is a peptidomimetic macrocycle of Table 8.

46. The peptidomimetic macrocycle of any one of claims 1-45, having Formula (I): 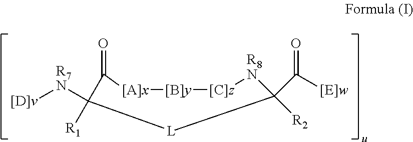
wherein:
each A, C, D, and E is independently an amino acid;
each B is independently an amino acid, 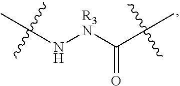
[—NH-L₃-CO—], [—NH-L₃-SO₂—], or [—NH-L3-];
each R₁ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or
at least one of R₁ and R₂ forms a macrocycle-forming linker L′ connected to the alpha position of one of said D or E amino acids;
each R₃ is independently —H, alkyl, alkenyl, alkynyl, arylalkyl, heteroalkyl, cycloalkyl, heterocycloalkyl, cycloalkylalkyl, aryl, or heteroaryl, optionally substituted with R₅;
each L and L′ is independently a macrocycle-forming linker of the formula -L₁-L₂-, 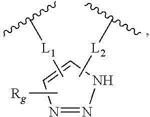
or -L₁-S-L2-S-L₃-;
each L₁, L₂ and L₃ is independently alkylene, alkenylene, alkynylene, heteroalkylene, cycloalkylene, heterocycloalkylene, arylene, heteroarylene, or [—R₄—K—R₄—]ₙ, each being optionally substituted with R₅;
when L is not 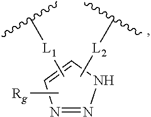
or -L₁-S-L₂-S-L₃-, L₁ and L₂ are alkylene, alkenylene, alkynylene, heteroalkylene, cycloalkylene, heterocycloalkylene, arylene, or heteroarylene;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂ or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each R₉ is independently alkyl, alkenyl, alkynyl, aryl, cycloalkyl, cycloalkenyl, heteroaryl, or heterocyclyl group, unsubstituted or optionally substituted with Rₐ and/or R_{b};
Rₐ and R_{b} are independently alkyl, OCH₃, CF₃, NH₂, CH₂NH₂, F, Br, I, 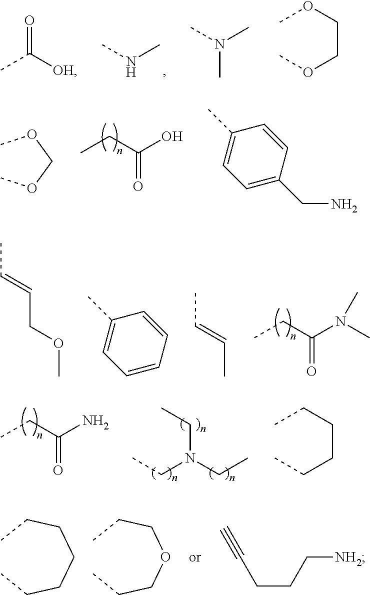
each v and w is independently an integer from 0-1000, for example 0-500, 0-200, 0-100, 0-50, 0-30, 0-20, or 0-10;
u is an integer from 1-10, for example 1-5, 1-3 or 1-2;
each x, y and z is independently an integer from 0-10, for example the sum of x+y+z is 2, 3, 6 or 10;
each n is independently an integer from 1-5; and
wherein A, B, C, D, and E, taken together with the crosslinked amino acids connected by the macrocycle-forming linker -L₁-L₂-, form an amino acid sequence of the peptidomimetic macrocycle with at least about 60%, 65%, 70%, 75%, 80%, 85%, 90%, 95%, 96%, 97%, 98%, 99% or 100% sequence identity to a sequence of Table 1a, 1b, 2a, or 2b.

47. The peptidomimetic macrocycle of claim 46, wherein an amino acid sequence of the peptidomimetic macrocycle has at least about 60%, 65%, 70%, 75%, 80%, 85%, 90%, 95%, 96%, 97%, 98%, 99% or 100% sequence identity to a sequence of Table 1a or 3a.

48. The peptidomimetic macrocycle of claim 46, wherein an amino acid sequence of the peptidomimetic macrocycle has at least about 60%, 65%, 70%, 75%, 80%, 85%, 90%, 95%, 96%, 97%, 98%, 99% or 100% sequence identity to a sequence of Table 6 or Table 7.

49. The peptidomimetic macrocycle of any one of claims 46-48, wherein u is 1.

50. The peptidomimetic macrocycle of any one of claims 46-49, wherein the sum of x+y+z is 2, 3 or 6.

51. The peptidomimetic macrocycle of any one of claims 46-50, wherein the sum of x+y+z is 3 or 6.

52. The peptidomimetic macrocycle of any one of claims 46-51, wherein each of v and w is independently an integer from 0-200.

53. The peptidomimetic macrocycle of any one of claims 46-52, wherein each of v and w is independently an integer from 0-10, 0-15, 0-20, 0-25, or 0-30.

54. The peptidomimetic macrocycle of any one of claims 46-53, wherein L₁ and L₂ are independently alkylene, alkenylene or alkynylene.

55. The peptidomimetic macrocycle of any one of claims 46-54, wherein L₁ and L₂ are independently C₃-C₁₀ alkylene or alkenylene.

56. The peptidomimetic macrocycle of any one of claims 46-55, wherein L₁ and L₂ are independently C₃-C₆ alkylene or alkenylene.

57. The peptidomimetic macrocycle of any one of claims 46-56, wherein L is

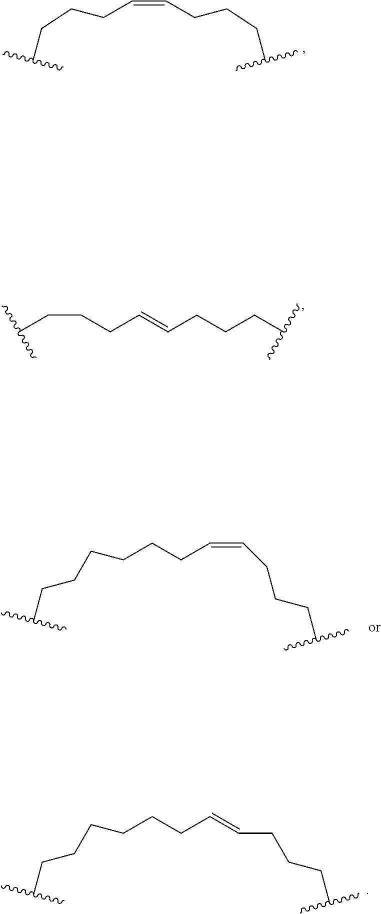 [SMILES: *CCC/C=C\CCC* ; *CCC/C=C/CCC* ; *CCC/C=C\CCCCCC* ; *CCC/C=C/CCCCCC*]

58. The peptidomimetic macrocycle of anyone of claims 46-56, wherein L is

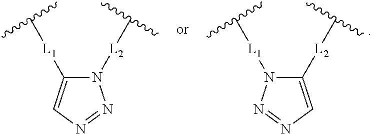

59. The peptidomimetic macrocycle of any one of claims 46-56 and 58, wherein L is

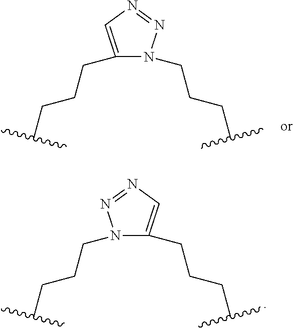 [SMILES: *CCCc1cnnn1CCC* ; *CCCc1cnnn1CCC*]

60. The peptidomimetic macrocycle of any one of claims 46-59, wherein R₁ and R₂ are H.

61. The peptidomimetic macrocycle of any one of claims 46-59, wherein R₁ and R₂ are independently alkyl.

62. The peptidomimetic macrocycle of any one of claims 46-59 and 61, wherein R₁ and R₂ are methyl.

63. The peptidomimetic macrocycle of any one of claims 46-62, having the Formula (Ia): 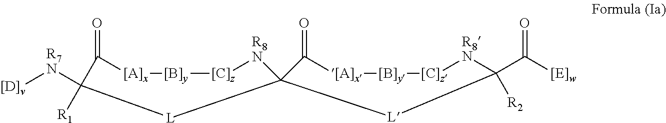
wherein:
R₈′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a E residue; and
x′, y′ and z′ are independently integers from 0-10.

64. The peptidomimetic macrocycle of any one of claims 46-48 and 50-62, wherein u is 2.

65. The peptidomimetic macrocycle of any one of claims 46-48 and 50-62, having the Formula (Ib): 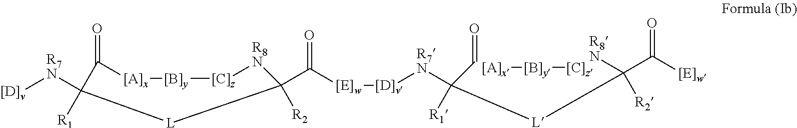
wherein:
R₇′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈′ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
v′ and w′ are independently integers from 0-100; and
x′, y′ and z′ are independently integers from 0-10, for example x′+y′+z′ is 2, 3, 6 or 10.

66. The peptidomimetic macrocycle of claim 64 or 65, wherein the sum of x+y+z is 2, 3 or 6, for example 3 or 6.

67. The peptidomimetic macrocycle of any one of claims 64-66, wherein the sum of x′+y′+z′ is 2, 3 or 6, for example 3 or 6.

68. The peptidomimetic macrocycle of any one of claims 64-67, wherein each of v and w is independently an integer from 1-10, 1-15, 1-20, or 1-25.

69. The peptidomimetic macrocycle of any one of claims 46-48 and 50-62, wherein u is 3.

70. The peptidomimetic macrocycle of claim 69, having the Formula (Ic): 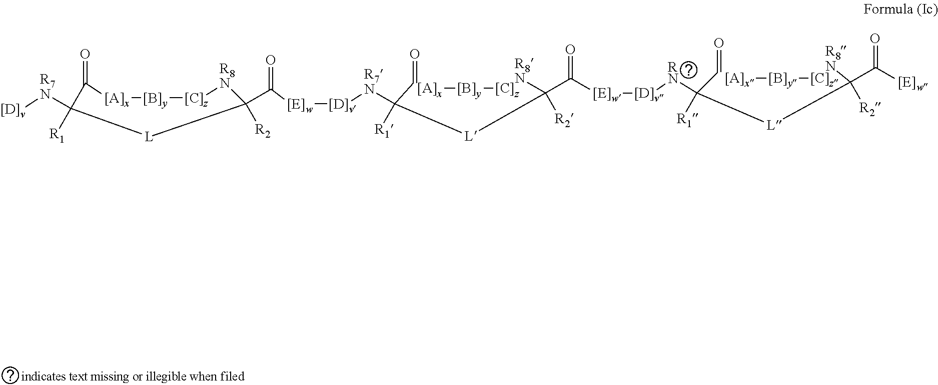
wherein:
R₇″ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈″ is —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
v″ and w″ are independently integers from 0-100; and
x″, y″ and z″ are independently integers from 0-10, for example x″+y″+z″ is 2, 3, 6 or 10.

71. The peptidomimetic macrocycle of any one of claims 2-45, having the Formula (IIIa) or Formula (IIIb): 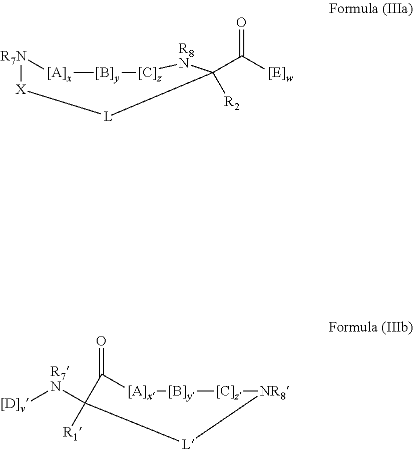
wherein:
each A, C, D and E is independently an amino acid;
each B is independently an amino acid, 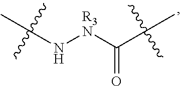
[—NH-L₃-CO—], [—NH-L₃-SO₂—], or [—NH-L3-];
each R₁′ and R₂ is independently —H, alkyl, alkenyl, alkynyl, arylalkyl, cycloalkyl, cycloalkylalkyl, heteroalkyl, or heterocycloalkyl, unsubstituted or substituted with halo-; or
R₂ forms a macrocycle-forming linker L′ connected to the alpha position of one of said E amino acids;
each R₃ is independently —H, alkyl, alkenyl, alkynyl, arylalkyl, heteroalkyl, cycloalkyl, heterocycloalkyl, cycloalkylalkyl, aryl, or heteroaryl, optionally substituted with R₅;
L and L′ are independently a macrocycle-forming linker of the formula -L₁-L₂-, 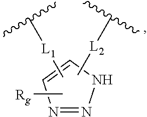
or -L₁-S-L2-S-L3-;
L₁, L₂ and L₃ are independently alkylene, alkenylene, alkynylene, heteroalkylene, cycloalkylene, heterocycloalkylene, arylene, heteroarylene, or [—R₄—K—R₄—]ₙ, each being optionally substituted with R₅;
each R₄ is independently alkylene, alkenylene, alkynylene, heteroalkylene, cycloalkylene, heterocycloalkylene, arylene, or heteroarylene;
each K is independently O, S, SO, SO₂, CO, CO₂ or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
R₇ or R₇′ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with a D residue;
R₈ or R₈′ is independently —H, alkyl, alkenyl, alkynyl, arylalkyl, cycloalkyl, heteroalkyl, cycloalkylalkyl, heterocycloalkyl, aryl, or heteroaryl, optionally substituted with R₅, or part of a cyclic structure with an E residue;
each R₉ is independently alkyl, alkenyl, alkynyl, aryl, cycloalkyl, cycloalkenyl, heteroaryl, or heterocyclyl group, unsubstituted or optionally substituted with Rₐ and/or R_{b};
each Rₐ and R_{b} is independently alkyl, OCH₃, CF₃, NH₂, CH₂NH₂, F, Br, I, 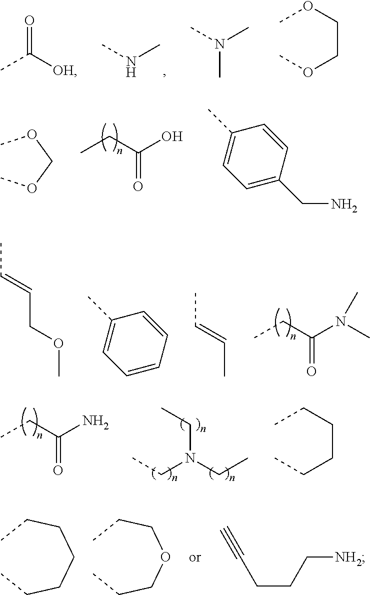
v and w′ are independently integers from 0-1000, for example 0-500, 0-200, 0-100, 0-50, 0-30, 0-20, or 0-10;
x, y, z, x′, y′ and z′ are independently integers from 0-10, for example the sum of x+y+z is 2, 3, 6 or 9, or the sum of x′+y′+z′ is 2, 3, 6, or 9;
n is an integer from 1-5;
X is C═O, CHR_{c}, or C═S;
R_{c} is alkyl, alkenyl, alkynyl, arylalkyl, cycloalkyl, cycloalkylalkyl, heteroalkyl, or heterocycloalkyl; and
A, B, C, and E, taken together with the crosslinked amino acids connected by the macrocycle-forming linker -L₁-L₂-, form an amino acid sequence of the peptidomimetic macrocycle with at least about 60%, 65%, 70%, 75%, 80%, 85%, 90%, 95%, 96%, 97%, 98%, 99%, or 100% sequence identity to a sequence of Table 1a, 1b, 2a, or 2b.

72. The peptidomimetic macrocycle of claim 71, wherein the amino acid sequence of the peptidomimetic macrocycle has at least about 60%, 65%, 70%, 75%, 80%, 85%, 90%, 95%, 96%, 97%, 98%, 99%, or 100% sequence identity to a sequence of Table 1a or 3a.

73. The peptidomimetic macrocycle of claim 71 or 72, having the Formula: 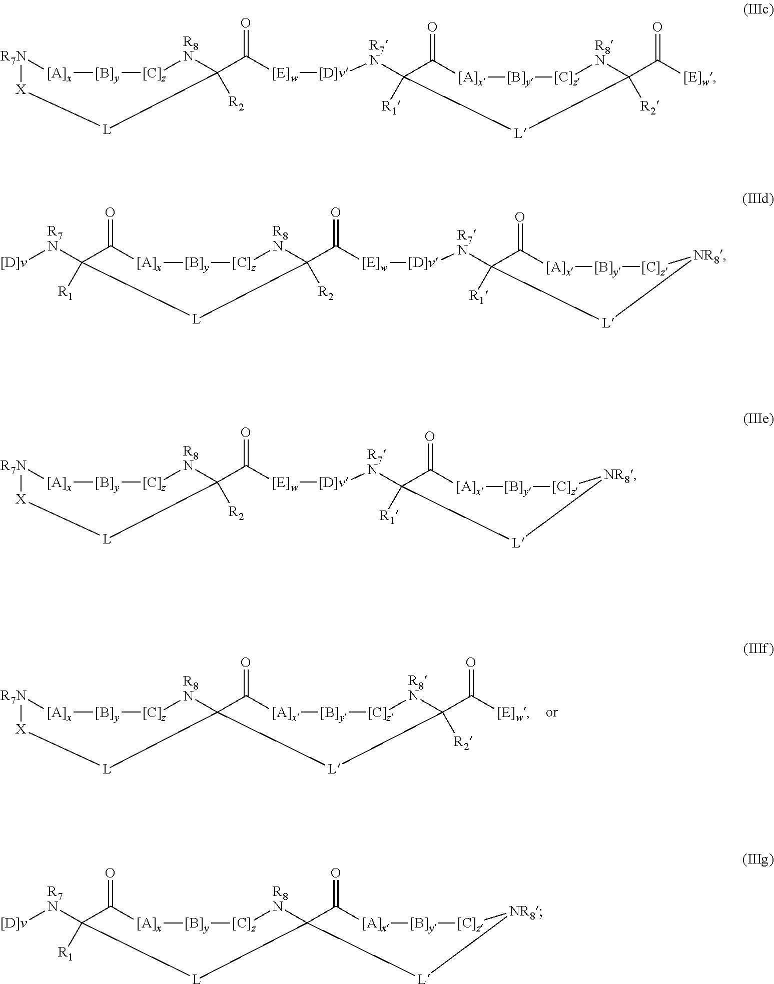
wherein
R₁′ and R₂′ are independently —H, alkyl, alkenyl, alkynyl, arylalkyl, cycloalkyl, cycloalkylalkyl, heteroalkyl, or heterocycloalkyl, unsubstituted or substituted with halo-; and
v, w, v′ and w′ are independently integers from 0-100.

74. The peptidomimetic macrocycle of any one of claims 71-73, wherein L₁ and L₂ are independently alkylene, alkenylene or alkynylene.

75. A peptidomimetic macrocycle comprising an amino acid sequence of formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
wherein:
X₀ is —H or an N-terminal capping group;
X₃₇ is —OH, or a C-terminal capping group;
X₁-X₃₆ are absent or are amino acids, such that at least X₇-X₂₂ are not absent;
at least three, four, five, six, or seven amino acids from the group consisting of X₂₀, X₂₃, X₂₄, X₂₅, X₂₇, X₂₈, X₃₁, X₃₂, and X₃₄ are selected as follows: X₂₀ is Arg, X₂₃ is Trp or Phe, X₂₄ is Leu, X₂₅ is Arg, X₂₇ is Lys or Leu, X₂₈ is Leu or Ile, X₃₁ is Val or Ile, X₃₂ is His, and X₃₄ is Phe;
and wherein the peptidomimetic macrocycle comprises at least one pair of crosslinked amino acids selected from the group consisting of amino acids X₁-X₃₆.

76. A peptidomimetic macrocycle comprising an amino acid sequence of formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
wherein:
X₀ is —H or an N-terminal capping group;
X₃₇ is —OH, or a C-terminal capping group;
X₁-X₃₆ are absent or are amino acids, such that at least X₇-X₂₂ are not absent;
at least three, four, five, six, or seven amino acids from the group consisting of X₂₀, X₂₃, X₂₄, X₂₅, X₂₇, X₂₈, X₃₁, X₃₂, and X₃₄ are selected as follows: X₂₀ is Arg or Cit or an analog thereof, X₂₃ is Trp or Phe or Ala or 1Nal or 2Nal, X₂₄ is Leu or Cpg or Cba or Ala or an analog thereof or a crosslinked amino acid, X₂₅ is Arg or His or Aib or Phe or Ser or Glu or Ala or Tyr or Trp or an analog thereof or a crosslinked amino acid, X₂₇ is Lys or Leu or Cit or Nle or hF or Tyr or His or Phe or Gln or an analog thereof or a crosslinked amino acid, X₂₈ is Leu or Ile or Cpg or Cba or Cha or an analog thereof or a crosslinked amino acid, X₃₁ is Val or Ile or Cpg or Cba or Nle or Thr or an analog thereof or a crosslinked amino acid, X₃₂ is His or Tyr or Phe or Ala or 2Pal or an analog thereof or a crosslinked amino acid, and X₃₄ is Phe or Tyr or Ala;
and wherein the peptidomimetic macrocycle comprises at least one pair of crosslinked amino acids selected from the group consisting of amino acids X₁-X₃₆.

77. A peptidomimetic macrocycle having the Formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
wherein:
X₀ is —H or an N-terminal capping group;
X₃₇ is —OH or a C-terminal capping group;
X₁-X₃₆ are absent or are amino acids, such that at least X₇-X₂₂ are not absent;
A is the amino acid sequence X₀-X₁-X₂-X₃-X₄-X₅-X₆-X₇-X₈-X₉-X₁₀-X₁₁-X₁₂-X₁₃-X₁₄ and comprises at least three amino acids selected from PTH (7-14);
B is the amino acid sequence X₁₅-X₁₆-X₁₇-X₁₈-X₁₉-X₂₀-X₂₁ and comprises at least three amino acids selected from PTHrP (15-21); and
C is the amino acid sequence X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇ and comprises at least six amino acids selected from PTH (22-34);
and wherein the peptidomimetic macrocycle comprises at least one pair of crosslinked amino acids selected from the group consisting of amino acids X₁-X₃₆.

78. A peptidomimetic macrocycle having the Formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
wherein:
X₀ is —H or an N-terminal capping group;
X₃₇ is —OH or a C-terminal capping group;
X₁-X₃₆ are absent or are amino acids, such that at least X₇-X₂₂ are not absent;
A is the amino acid sequence X₀-X₁-X₂-X₃-X₄-X₅-X₆-X₇-X₈-X₉-X₁₀-X₁₁-X₁₂-X₁₃-X₁₄ and comprises at least two amino acids selected from PTHrP (7-14);
B is the amino acid sequence X₁₅-X₁₆-X₁₇-X₁₈-X₁₉-X₂₀-X₂₁ and comprises at least three amino acids selected from PTHrP (15-21); and
C is the amino acid sequence X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇ and comprises at least three amino acids selected from PTH (22-34); and wherein the peptidomimetic macrocycle comprises at least one pair of crosslinked amino acids selected from the group consisting of amino acids X₁-X₃₆.

79. A peptidomimetic macrocycle having the Formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
wherein:
X₀ is —H or an N-terminal capping group;
X₃₇ is —OH or a C-terminal capping group;
X₁-X₃₆ are absent or are amino acids, such that at least X₇-X₂₂ are not absent;
A is the amino acid sequence X₀-X₁-X₂-X₃-X₄-X₅-X₆-X₇-X₈-X₉-X₁₀-X₁₁-X₁₂-X₁₃-X₁₄ and comprises at least three amino acids selected from PTHrP (7-14) or at least three amino acids selected from PTHrP (7-14); wherein X₁₀ is not Asn or Asp; X₁₁ is not Asn or Asp, X₁₂ is not Gly, or any combination thereof;
B is the amino acid sequence X₁₅-X₁₆-X₁₇-X₁₈-X₁₉-X₂₀-X₂₁ and comprises at least three amino acids selected from PTHrP (15-21); and
C is the amino acid sequence X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇ and comprises at least three amino acids selected from PTHrP (22-36) or at least three amino acids selected from PTH (22-34); and
wherein the peptidomimetic macrocycle comprises at least one pair of crosslinked amino acids selected from the group consisting of amino acids X₁-X₃₆.

80. A peptidomimetic macrocycle having the Formula:
X₀-X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇
wherein:
X₀ is —H or an N-terminal capping group;
X₃₇ is —OH or a C-terminal capping group;
X₁-X₃₆ are absent or are amino acids, such that at least X₇-X₂₂ are not absent;
A is the amino acid sequence X₀-X₁-X₂-X₃-X₄-X₅-X₆-X₇-X₈-X₉-X₁₀-X₁₁-X₁₂-X₁₃-X₁₄ and comprises at least two contiguous amino acids selected from PTHrP (7-14);
B is the amino acid sequence X₁₅-X₁₆-X₁₇-X₁₈-X₁₉-X₂₀-X₂₁ and comprises at least three contiguous amino acids selected from PTHrP (15-21); and
C is the amino acid sequence X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-X₃₇ and comprises at least two contiguous amino acids selected from PTHrP (22-36) or at least two contiguous amino acids selected from PTH (22-34); and
wherein the peptidomimetic macrocycle comprises at least one pair of crosslinked amino acids selected from the group consisting of amino acids X₁-X₃₆.

81. The peptidomimetic macrocycle of any one of claims 75-80, wherein
X₀ is —H or an N-terminal capping group, for example acetyl, 1NaAc, 2NaAc, PhAc, a fatty acid, a urea, a sulfonamide, or a polyalkylene oxide linked to the N-terminus of residue X₁;
X₁ is Ser, Ala, Deg, Har, a dialkylated amino acid, Aib, Ac5c, Ac3c, Ac6c, desamino-Ser, desamino-Ac5c, desamino-Aib, Val, an analog thereof, or absent;
X₂ is an aromatic amino acid, Val, Trp, Arg, D-Trp, D-Arg, F4COOH, Bip, F4NH₂, 1Nal, 2Nal, 2Pal, 3Pal, 4Pal, Bpa, Deg, Ile, an analog thereof, or absent;
X₃ is Ser, Deg, Aib, Ac3c, Ac5c, Ac6c, Glu, Lys, Phe, Aib, Gly, Ala, an analog thereof, or absent;
X₄ is Glu, Gln, Phe, His, an analog thereof, or absent;
X₅ is Ile, His, Lys, Glu, Phe, an analog thereof, or absent;
X₆ is Gln, Lys, Glu, Phe, Ala, an analog thereof, or absent;
X₇ is an aromatic amino acid, a hydrophobic amino acid, Leu, Lys, Glu, Ala, Phe, Met,
F4Cl, 1Nal, 2Nal, 2Pal, 3Pal, 4Pal, Phe, Nle, an analog thereof, or a crosslinked amino acid;
X₈ is a hydrophobic amino acid, Met, Leu, Nle, an analog thereof, or a crosslinked amino acid;
X₉ is an aromatic amino acid, His, Aib, or an analog thereof;
X₁₀ is Asn, Asp, Gln, Ala, Ser, Val, His, Trp, Aib, an analog thereof, or a crosslinked amino acid;
X₁₁ is a hydrophobic amino acid, a positively charged amino acid, an aromatic amino acid, Leu, Lys, Har, Arg, Ala, Val, Ile, Met, Phe, Trp, D-Trp, Nle, Cit, hK, hL, an analog thereof, or a crosslinked amino acid;
X₁₂ is a D-amino acid, a hydrophobic amino acid, a hydrophilic amino acid, an aromatic amino acid, a positively charged amino acid, a negatively charged amino acid, an uncharged amino acid, Gly, D-Trp, Ala, Aib, Arg, His, Trp, an analog thereof, or a crosslinked amino acid;
X₁₃ is a positively charged amino acid, Lys, Ser, Ala, Aib, Leu, Glu, Gln, Arg, His, Phe, Trp, Pro, Cit, Kfam, Ktam, an analog thereof, or a crosslinked amino acid;
X₁₄ is an aromatic amino acid, His, Ser, Trp, Ala, Leu, Lys, Arg, Phe, Trp, Aib, an analog thereof, or a crosslinked amino acid;
X₁₅ is a hydrophobic amino acid, Leu, Ile, Tyr, Aib, an analog thereof, or a crosslinked amino acid;
X₁₆ is Asn, Gln, Lys, Ala, Glu, an analog thereof, or a crosslinked amino acid;
X₁₇ is Ser, Asp, β-Ala, β-hPhe, Aib, an analog thereof, or a crosslinked amino acid;
X₁₈ is a hydrophobic amino acid, Met, Nle, Leu, β-hIle, hSer(OMe), β-hPhe, Aib, an analog thereof, or a crosslinked amino acid;
X₁₉ is a positively charged amino acid, Glu, Arg, Ser, Aib, Cit, Glu, Ala, an analog thereof, or a crosslinked amino acid;
X₂₀ is a positively charged amino acid, Cit, Arg, Ala, an analog thereof, or a crosslinked amino acid;
X₂₁ is a positively charged amino acid, Cit, Val, Arg, Lys, Gln, Cit, Ala, an analog thereof, or a crosslinked amino acid;
X₂₂ is an aromatic amino acid, Glu, Phe, Ser, Aib, an analog thereof, or a crosslinked amino acid;
X₂₃ is an aromatic amino acid, a hydrophobic amino acid, Trp, Phe, Ala, 9-Aal, 1Nal, 2Nal, an analog thereof, absent, or a crosslinked amino acid;
X₂₄ is an aromatic amino acid, a hydrophobic amino acid, Leu, Ala, Cba, Cpg, Aib, an analog thereof, absent, or a crosslinked amino acid;
X₂₅ is a positively charged amino acid, Cit, Arg, His, Leu, Trp, Tyr, Phe, Ala, Ser, Glu, Aib, an analog thereof, absent, or a crosslinked amino acid;
X₂₆ is a positively charged amino acid, Lys, His, Ala, Phe, Ser, Glu, AmO, AmK, Cit, and Aib an analog thereof, absent, or a crosslinked amino acid;
X₂₇ is a positively charged amino acid, Cit, Lys, Leu, Arg, Nle, Tyr, His, Phe, hF, Leu, Gln, an analog thereof, absent, or a crosslinked amino acid;
X₂₈ is an aromatic amino acid, a hydrophobic amino acid, Leu, Ile, Cba, Cha, Cpg, Aib, an analog thereof, absent, or a crosslinked amino acid;
X₂₉ is Gln, Ala, Glu, Ser, Aib, an analog thereof, absent, or a crosslinked amino acid;
X₃₀ is Asp, Glu, Leu, Arg, hPhe, Asn, His, Ser, Ala, Phe, an analog thereof, absent, or a crosslinked amino acid;
X₃₁ is an aromatic amino acid, a hydrophobic amino acid, Val, Ile, Nle, Thr, Ser, Cba, Cpg, an analog thereof, absent, or a crosslinked amino acid;
X₃₂ is an aromatic amino acid, His, Trp, Arg, Phe, Tyr, Ile, Ala, 2Pal, 3Pal, 4Pal, an analog thereof, absent, or a crosslinked amino acid;
X₃₃ is Asn, Thr, Glu, Asp, Lys, Phe, an analog thereof, absent, or a crosslinked amino acid;
X₃₄ is an aromatic amino acid, a hydrophobic amino acid, Phe, Ala, Tyr, Arg, 2Nal, hF, Glu, Lys, Ser, an analog thereof, absent, or a crosslinked amino acid;
X₃₅ is Glu, Gly, an analog thereof, absent, or a crosslinked amino acid;
X₃₆ is an aromatic amino acid, Tyr, Pra, an analog thereof, absent, or a crosslinked amino acid; and
X₃₇ is —OH, or a C-terminal capping group, for example a primary, secondary, or tertiary amino group, an alkyloxy or an aryloxy group.

82. The peptidomimetic macrocycle of any one of claims 75-81, wherein:
X₀ is —H or an N-terminal capping group, for example acetyl, 1NaAc, 2NaAc, PhAc, a fatty acid, a urea, a sulfonamide, or a polyalkylene oxide linked to the N-terminus of residue X₁;
X₁ is Ser, Ala, Deg, Har, a dialkylated amino acid, Aib, Ac5c, Ac3c, Ac6c, desamino-Ser, desamino-Ac5c, desamino-Aib, Val, an analog thereof, or absent;
X₂ is an aromatic amino acid, Val, Trp, Arg, D-Trp, D-Arg, F4COOH, Bip, F4NH₂, 1Nal, 2Nal, 2Pal, 3Pal, 4Pal, Bpa, Deg, Ile, an analog thereof, or absent;
X₃ is Ser, Deg, Aib, Ac3c, Ac5c, Ac6c, Glu, Lys, Phe, Aib, Gly, Ala, an analog thereof, or absent;
X₄ is Glu, Gln, Phe, His, an analog thereof, or absent;
X₅ is Ile, His, Lys, Glu, Phe, an analog thereof, or absent;
X₆ is Gln, Lys, Glu, Phe, Ala, an analog thereof, or absent;
X₇ is an aromatic amino acid, a hydrophobic amino acid, Leu, Lys, Glu, Ala, Phe, F4Cl, 1Nal, 2Nal, 2Pal, 3Pal, 4Pal, Phe, or an analog thereof;
X₈ is a hydrophobic amino acid, Met, Leu, Nle, or an analog thereof;
X₉ is an aromatic amino acid, His, or an analog thereof;
X₁₀ is Asn, Asp, Gln, Ala, Ser, Val, His, Trp, an analog thereof, or a crosslinked amino acid;
X₁₁ is a hydrophobic amino acid, a positively charged amino acid, an aromatic amino acid, Leu, Lys, Har, Arg, Ala, Val, Ile, Met, Phe, Trp, D-Trp or an analog thereof;
X₁₂ is a D-amino acid, a hydrophobic amino acid, a hydrophilic amino acid, an aromatic amino acid, a positively charged amino acid, a negatively charged amino acid, an uncharged amino acid, Gly, D-Trp, Ala, Aib, Arg, His, Trp or an analog thereof;
X₁₃ is a positively charged amino acid, Lys, Ser, Ala, Aib, Leu, Glu, Gln, Arg, His, Phe, Trp, Pro or an analog thereof;
X₁₄ is an aromatic amino acid, His, Ser, Trp, Ala, Leu, Lys, Arg, Phe, Trp, an analog thereof, or a crosslinked amino acid;
X₁₅ is a hydrophobic amino acid, Leu, Ile, Tyr, an analog thereof, or a crosslinked amino acid;
X₁₆ is Asn, Gln, Lys, an analog thereof, or a crosslinked amino acid;
X₁₇ is Ser, Asp, β-Ala, β-hPhe, an analog thereof, or a crosslinked amino acid;
X₁₈ is a hydrophobic amino acid, Met, Nle, Leu, β-hIle, hSer(OMe), β-hPhe, an analog thereof, or a crosslinked amino acid;
X₁₉ is a positively charged amino acid, Cit, Glu, Arg, Ser, an analog thereof, or a crosslinked amino acid;
X₂₀ is a positively charged amino acid, Cit, Arg, an analog thereof, or a crosslinked amino acid;
X₂₁ is a positively charged amino acid, Cit, Val, Arg, Lys, Gln, an analog thereof, or a crosslinked amino acid;
X₂₂ is an aromatic amino acid, Glu, Phe, an analog thereof, or a crosslinked amino acid;
X₂₃ is an aromatic amino acid, a hydrophobic amino acid, Trp, Phe, 9-Aal, 1Nal, 2Nal, an analog thereof, absent, or a crosslinked amino acid;
X₂₄ is an aromatic amino acid, a hydrophobic amino acid, Leu, an analog thereof, absent, or a crosslinked amino acid;
X₂₅ is a positively charged amino acid, Cit, Arg, His, Leu, Trp, Tyr, Phe, an analog thereof, absent, or a crosslinked amino acid;
X₂₆ is a positively charged amino acid, Lys, His, an analog thereof, absent, or a crosslinked amino acid;
X₂₇ is a positively charged amino acid, Cit, Lys, Leu, Arg, Nle, Tyr, His, Phe, hF, Leu, Gln, an analog thereof, absent, or a crosslinked amino acid;
X₂₈ is an aromatic amino acid, a hydrophobic amino acid, Leu, Ile, an analog thereof, absent, or a crosslinked amino acid;
X₂₉ is Gln, Ala, Glu, an analog thereof, absent, or a crosslinked amino acid;
X₃₀ is Asp, Glu, Leu, Arg, hPhe, Asn, His, Ser, an analog thereof, absent, or a crosslinked amino acid;
X₃₁ is an aromatic amino acid, a hydrophobic amino acid, Val, Ile, Nle, Thr, Ser, an analog thereof, absent, or a crosslinked amino acid;
X₃₂ is an aromatic amino acid, His, Trp, Arg, Phe, Tyr, Ile, 2Pal, 3Pal, 4Pal, an analog thereof, absent, or a crosslinked amino acid;
X₃₃ is Asn, Thr, Glu, Asp, Lys, an analog thereof, absent, or a crosslinked amino acid;
X₃₄ is an aromatic amino acid, a hydrophobic amino acid, Phe, Ala, Tyr, Arg, 2Nal, hF, Glu, Lys, an analog thereof, absent, or a crosslinked amino acid;
X₃₅ is Glu, an analog thereof, absent, or a crosslinked amino acid;
X₃₆ is an aromatic amino acid, Tyr, an analog thereof, absent, or a crosslinked amino acid; and
X₃₇ is —OH, or a C-terminal capping group, for example a primary, secondary, or tertiary amino group, an alkyloxy or an aryloxy group.

83. The peptidomimetic macrocycle of any one of claims 75-82, wherein the peptidomimetic macrocycle comprises at least one macrocycle-forming linker, wherein a macrocycle-forming linker of the at least one macrocycle-forming linker connects the at least one pair of crosslinked amino acids.

84. The peptidomimetic macrocycle of claim 83, wherein the at least one pair of crosslinked amino acids is selected from the group consisting of amino acids X₇-X₃₄.

85. The peptidomimetic macrocycle of claim 83 or 84, wherein the at least one macrocycle-forming linker connects amino acids X₉ and X₁₃.

86. The peptidomimetic macrocycle of any one of claims 83-85, wherein the at least one macrocycle-forming linker connects amino acids X₁₀ and X₁₄.

87. The peptidomimetic macrocycle of any one of claims 83-86, wherein the at least one macrocycle-forming linker connects amino acids X₁₁ and X₁₅.

88. The peptidomimetic macrocycle of any one of claims 83-87, wherein the at least one macrocycle-forming linker connects amino acids X₁₂ and X₁₆.

89. The peptidomimetic macrocycle of any one of claims 83-88, wherein the at least one macrocycle-forming linker connects amino acids X₁₃ and X₁₇.

90. The peptidomimetic macrocycle of any one of claims 83-89, wherein the at least one macrocycle-forming linker connects amino acids X₁₄ and X₁₈.

91. The peptidomimetic macrocycle of any one of claims 83-90, wherein the at least one macrocycle-forming linker connects amino acids X₁₈ and X₂₂.

92. The peptidomimetic macrocycle of any one of claims 83-91, wherein the at least one macrocycle-forming linker connects amino acids X₂₂ and X₂₆.

93. The peptidomimetic macrocycle of any one of claims 83-92, wherein the at least one macrocycle-forming linker connects amino acids X₂₄ and X₂₈

94. The peptidomimetic macrocycle of any one of claims 83-93, wherein the at least one macrocycle-forming linker connects amino acids X₂₆ and X₃₀.

95. The peptidomimetic macrocycle of any one of claims 83-94, wherein the at least one macrocycle-forming linker connects amino acids X₂₇ and X₃₁.

96. The peptidomimetic macrocycle of any one of claims 75-95, wherein the at least one macrocycle-forming linker comprises a first macrocycle-forming linker that connects a first pair of the at least one pair of crosslinked amino acids, and a second macrocycle-forming linker that connects a second pair of the at least one pair of crosslinked amino acids.

97. The peptidomimetic macrocycle of claim 96, wherein X₁₄ and X₁₈ are crosslinked amino acids, and X₂₆ and X₃₀ are crosslinked amino acids.

98. The peptidomimetic macrocycle of claim 96 or 97, wherein X₁₄ and X₁₈ are crosslinked amino acids, and X₂₂ and X₂₆ are crosslinked amino acids.

99. The peptidomimetic macrocycle of any one of claims 96-98, wherein X₁₄ and X₁₈ are crosslinked amino acids, and X₂₄ and X₂₈ are crosslinked amino acids.

100. The peptidomimetic macrocycle of any one of claims 96-99, wherein X₁₄ and X₁₈ are crosslinked amino acids, and X₂₇ and X₃₁ are crosslinked amino acids.

101. The peptidomimetic macrocycle of any one of claims 96-100, wherein X₁₃ and X₁₇ are crosslinked amino acids, and X₂₆ and X₃₀ are crosslinked amino acids.

102. The peptidomimetic macrocycle of any one of claims 75-101, wherein X₁-X₆ are absent.

103. The peptidomimetic macrocycle of any one of claims 75-102, wherein X₃₅-X₃₆ are absent.

104. The peptidomimetic macrocycle of any one of claims 75-103, wherein each of X₇, X₈, and X₉ is independently a crosslinked amino acid or any amino acid that is a same amino acid at a corresponding position of PTHrP.

105. The peptidomimetic macrocycle of any one of claims 75-104, wherein each of X₇, X₉, X₁₃, X₂₀, X₂₄, and X₃₂ is independently a crosslinked amino acid or any amino acid that is a same amino acid at a corresponding position of PTH and PTHrP

106. The peptidomimetic macrocycle of any one of claims 75-105, wherein X₁₀ is crosslinked or any amino acid except Asn or Asp.

107. The peptidomimetic macrocycle of any one of claims 75-106, wherein X₁₀ is Gln, Aib, Ala, or Glu.

108. The peptidomimetic macrocycle of any one of claims 75-107, wherein each of X₁₀, X₁₁, X₁₂, X₁₃, and X₁₄ is independently a crosslinked amino acid or any amino acid that is not a same amino acid at a corresponding position of PTH or PTHrP.

109. The peptidomimetic macrocycle of any one of claims 75-108, wherein X₁₁ is crosslinked or any amino acid except Leu or Lys.

110. The peptidomimetic macrocycle of any one of claims 75-108, wherein X₁₁ is Leu.

111. The peptidomimetic macrocycle of any one of claims 75-109, wherein X₁₁ is Arg or hArg.

112. The peptidomimetic macrocycle of any one of claims 75-109, wherein X₁₁ is Har.

113. The peptidomimetic macrocycle of any one of claims 75-112, wherein X₁₂ is crosslinked or any amino acid except Gly.

114. The peptidomimetic macrocycle of any one of claims 75-113, wherein X₁₂ is Ala or Aib.

115. The peptidomimetic macrocycle of any one of claims 75-114, wherein X₁₃ is crosslinked or any amino acid except Gly.

116. The peptidomimetic macrocycle of any one of claims 75-115, wherein X₁₃ is Lys or crosslinked.

117. The peptidomimetic macrocycle of any one of claims 75-116, wherein X₁₄ is crosslinked or any amino acid except His or Ser.

118. The peptidomimetic macrocycle of any one of claims 75-117, wherein X₁₄ is a hydrophobic amino acid.

119. The peptidomimetic macrocycle of claim 118, wherein the hydrophobic amino acid is a large hydrophobic amino acid.

120. The peptidomimetic macrocycle of claim 118 or 119, wherein X₁₄ is Trp or Phe.

121. The peptidomimetic macrocycle of any one of claims 118-120, wherein X₁₄ is Phe.

122. The peptidomimetic macrocycle of any one of claims 118-120, wherein X₁₄ is Tyr.

123. The peptidomimetic macrocycle of any one of claims 75-117, wherein X₁₄ is crosslinked.

124. The peptidomimetic macrocycle of any one of claims 75-123, wherein each of X₁₅-X₃₆ is independently a crosslinked amino acid or any amino acid that is a same amino acid at a corresponding position of PTHrP.

125. The peptidomimetic macrocycle of any one of claims 75-124, wherein each of X₁₃-X₃₆ is independently a crosslinked amino acid or any amino acid that is a same amino acid at a corresponding position of PTHrP.

126. The peptidomimetic macrocycle of any one of claims 75-125, wherein each of X₁₅, X₁₆, X₁₇, X₁₈, and X₁₉ is independently a crosslinked amino acid or any amino acid that is a same amino acid at a corresponding position of PTHrP.

127. The peptidomimetic macrocycle of any one of claims 75-126, wherein X₁₈ is a crosslinked amino acid.

128. The peptidomimetic macrocycle of any one of claims 75-127, wherein X₁₉ is a positively charged amino acid, Cit, Arg. or an analog thereof.

129. The peptidomimetic macrocycle of any one of claims 75-128, wherein X₁₉ is Arg.

130. The peptidomimetic macrocycle of claim 75-129, wherein X₂₀ is a positively charged amino acid, Cit, Arg, or an analog thereof.

131. The peptidomimetic macrocycle of any one of claims 75-130, wherein X₂₀ is Arg.

132. The peptidomimetic macrocycle of any one of claims 75-131, wherein X₂₁ is a positively charged amino acid, Cit, Arg, Lys, or an analog thereof.

133. The peptidomimetic macrocycle of any one of claims 75-132, wherein X₂₁ is Arg.

134. The peptidomimetic macrocycle of any one of claims 75-133, wherein at least two of X₁₉, X₂₀, and X₂₁ comprise a same amino acid at a corresponding position of from PTHrP.

135. The peptidomimetic macrocycle of claim of any one of claims 75-134, wherein X₁₉-X₂₀-X₂₁ is Arg-Arg-Arg.

136. The peptidomimetic macrocycle of any one of claims 75-135, wherein an amino acid of the at least one pair of crosslinked amino acids is X₂₂.

137. The peptidomimetic macrocycle of any one of claims 75-136, wherein X₂₃ is Trp.

138. The peptidomimetic macrocycle of any one of claims 75-136, wherein X₂₃ is Phe.

139. The peptidomimetic macrocycle of any one of claims 75-138, wherein X₂₄ is Leu.

140. The peptidomimetic macrocycle of any one of claims 75-139, wherein X₂₅ is Arg.

141. The peptidomimetic macrocycle of any one of claims 75-140, wherein X₂₆ is any amino acid except Lys or His.

142. The peptidomimetic macrocycle of any one of claims 75-141, wherein X₂₆ is Aib.

143. The peptidomimetic macrocycle of any one of claims 75-141, wherein X₂₆ is Glu.

144. The peptidomimetic macrocycle of any one of claims 75-143, wherein X₂₇ is Lys.

145. The peptidomimetic macrocycle of any one of claims 75-143, wherein X₂₇ is Leu.

146. The peptidomimetic macrocycle of any one of claims 75-145, wherein X₂₈ is Leu.

147. The peptidomimetic macrocycle of any one of claims 75-145, wherein X₂₈ is Ile.

148. The peptidomimetic macrocycle of any one of claims 75-147, wherein X₂₉ is Aib.

149. The peptidomimetic macrocycle of any one of claims 75-148, wherein X₃₁ is Val.

150. The peptidomimetic macrocycle of any one of claims 75-148, wherein X₃₁ is Ile.

151. The peptidomimetic macrocycle of any one of claims 75-150, wherein X₃₂ is His.

152. The peptidomimetic macrocycle of any one of claims 75-151, wherein X₃₃ is Glu.

153. The peptidomimetic macrocycle of any one of claims 75-151, wherein X₃₃ is Asn.

154. The peptidomimetic macrocycle of any one of claims 75-151, wherein X₃₃ is Aib or Cit.

155. The peptidomimetic macrocycle of any one of claims 75-154, wherein X₃₄ is Phe.

156. The peptidomimetic macrocycle of any one of claims 75-155, wherein X₂₀ is Arg, X₂₃ is Trp, X₂₄ is Leu, X₂₅ is Arg, X₂₇ is Lys, X₂₈ is Leu, X₃₁ is Val, and X₃₄ is Phe.

157. The peptidomimetic macrocycle of any one of claims 75-155, wherein X₂₀ is Arg, X₂₃ is Phe, X₂₄ is Leu, X₂₇ is Leu, X₂₈ is Ile, X₃₁ is Ile, and X₃₂ is His.

158. The peptidomimetic macrocycle of any one of claims 75-157, wherein the macrocycle comprises a contiguous amino acid sequence comprising at least 3 contiguous amino acids that are crosslinked amino acids or same amino acids as those at corresponding positions of PTH.

159. The peptidomimetic macrocycle of any one of claims 75-158, wherein the macrocycle comprises a contiguous amino acid sequence comprising at least 3 contiguous amino acids that are crosslinked amino acids or same amino acids as those at corresponding positions of PTHrP.

160. The peptidomimetic macrocycle of any one of claims 75-159, wherein the macrocycle comprises a contiguous amino acid sequence comprising at most 13 amino acids that are crosslinked amino acids or same amino acids as those at corresponding positions of PTH.

161. The peptidomimetic macrocycle of claim 160, wherein the macrocycle comprises a substitution within the contiguous amino acid sequence comprising at most 13 amino acids that are crosslinked amino acids or same amino acids as those at corresponding positions of PTH.

162. The peptidomimetic macrocycle of claim 161, wherein the substitution is at X₂₆.

163. The peptidomimetic macrocycle of claim 161 or 162, wherein the substitution is at X₂₉.

164. The peptidomimetic macrocycle of any one of claims 161-163, wherein the substitution is at X₃₃.

165. The peptidomimetic macrocycle of any one of claims 75-164, wherein the macrocycle comprises at most 10 amino acids that are crosslinked or substitutions, wherein the substitutions are not same amino acids as those at corresponding positions of PTHrP or PTH.

166. The peptidomimetic macrocycle of any one of claims 75-165, wherein the macrocycle comprises 2 or 4 crosslinked amino acids and at least 3 amino acids that are not same amino acids as those at corresponding positions of PTHrP or PTH.

167. The peptidomimetic macrocycle of any one of claims 75-166, wherein the macrocycle comprises 3, 4, 5, 6, 7, 8, 9 or 10 amino acids that are crosslinked or substitutions, wherein the substitutions are not same amino acids as those at corresponding positions of PTHrP or PTH.

168. A peptidomimetic macrocycle selected from Table 3.

169. A peptidomimetic macrocycle selected from Table 7.

170. A peptidomimetic macrocycle selected from Table 6.

171. A peptidomimetic macrocycle selected from Table 8.

172. The peptidomimetic macrocycle of any one of claims 1-171, comprising a helix.

173. The peptidomimetic macrocycle of any one of claims 1-172, comprising an α-helix.

174. The peptidomimetic macrocycle of any one of claims 1-173, comprising an α,α-disubstituted amino acid.

175. The peptidomimetic macrocycle of claim 174, wherein each amino acid connected by the at least one macrocycle-forming linker is an α,α-disubstituted amino acid.

176. The peptidomimetic macrocycle of any one of claims 1-175, wherein the at least one macrocycle-forming linker is a straight chain alkenyl.

177. The peptidomimetic macrocycle of any one of claims 1-176, wherein the at least one macrocycle-forming linker is a straight chain alkenyl with 6 to 14 carbon atoms.

178. The peptidomimetic macrocycle of any one of claims 1-177, wherein the at least one macrocycle-forming linker is a straight chain alkenyl with 8 to 12 carbon atoms, for example 8, 9, 10, 11 or 12 carbon atoms.

179. The peptidomimetic macrocycle of any one of claims 1-178, wherein the at least one macrocycle-forming linker is a C₈ alkenyl with a double bond between C₄ and C₅ of the C₈ alkenyl.

180. The peptidomimetic macrocycle of any one of claims 1-178, wherein the at least one macrocycle-forming linker is a C₁₂ alkenyl with a double bond between C₄ and C₅ or C₅ and C₆ of the C₁₂ alkenyl.

181. The peptidomimetic macrocycle of any one of claims 1-180, wherein the at least one macrocycle-forming linker comprises at least 2 macrocycle-forming linkers.

182. The peptidomimetic macrocycle of any one of claims 1-181, wherein the at least one macrocycle-forming linker comprises a first and a second macrocycle-forming linker, wherein the first macrocycle-forming linker connects a first and a second amino acid, wherein the second macrocycle-forming linker connects a third and a fourth amino acid, wherein the first amino acid is upstream of the second amino acid, the second amino acid is upstream of the third amino acid, and the third amino acid is upstream of the fourth amino acid.

183. The peptidomimetic macrocycle of claim 182, wherein 1, 2, 3, 4, 5, 6, or 7, amino acids are between the second and third amino acids.

184. The peptidomimetic macrocycle of claim 182 or 183, wherein 4 or 5 amino acids are between the second and third amino acids.

185. The peptidomimetic macrocycle of any one of claims 1-184, wherein the peptidomimetic macrocycle contains 16-36 amino acids, for example 16, 17, 18, 19, 20, 21, 22, 23, 24, 25, 26, 27, 28, 29, 30, 31, 32, 33, 34, 35 or 36 amino acids.

186. The peptidomimetic macrocycle of any one of claims 1-185, wherein the peptidomimetic macrocycle contains 24-36 amino acids, for example 24, 25, 26, 27, 28, 29, 30, 31, 32, 33, 34, 35 or 36 amino acids.

187. A peptidomimetic macrocycle, wherein the peptidomimetic macrocycle is

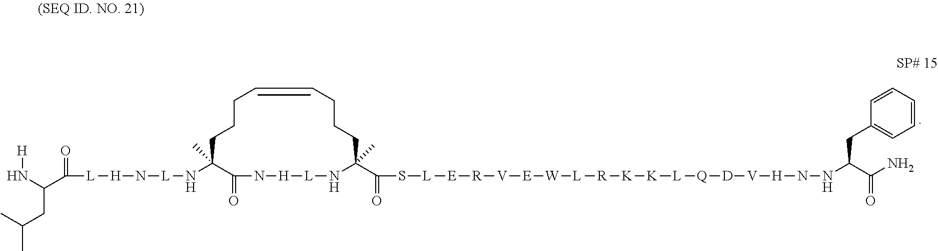

188. A peptidomimetic macrocycle, wherein the peptidomimetic macrocycle is

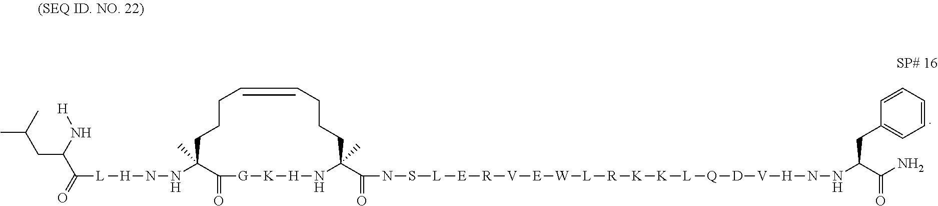

189. A peptidomimetic macrocycle, wherein the peptidomimetic macrocycle is

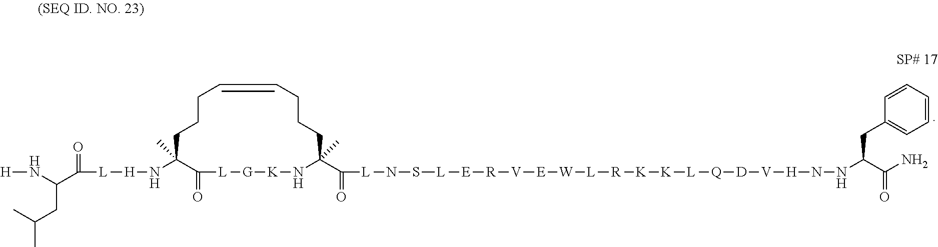

190. A peptidomimetic macrocycle, wherein the peptidomimetic macrocycle is

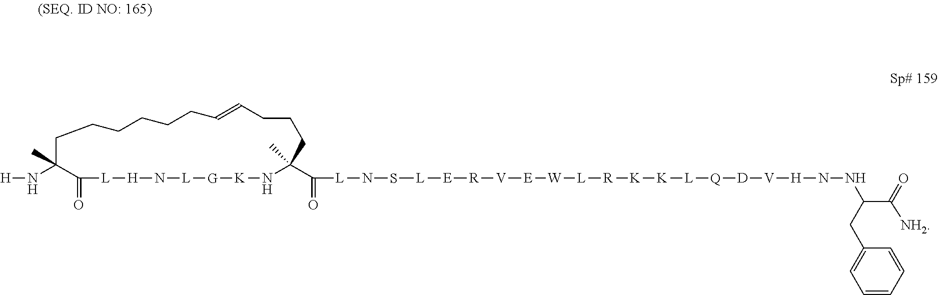

191. A peptidomimetic macrocycle, wherein the peptidomimetic macrocycle is

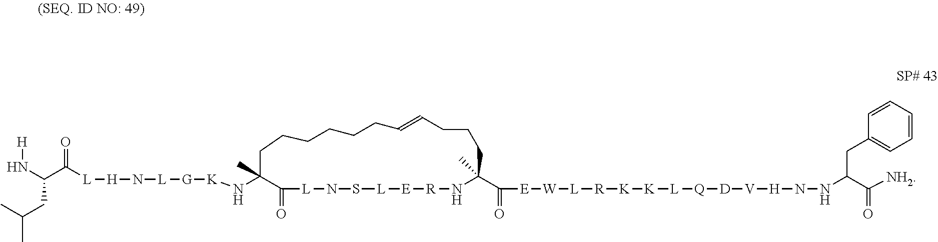

192. A peptidomimetic macrocycle, wherein the peptidomimetic macrocycle is

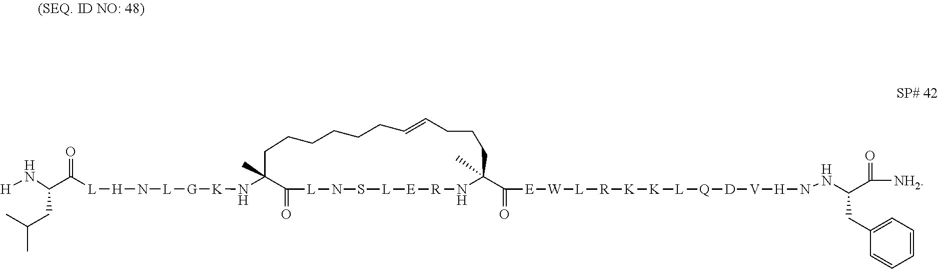

193. A peptidomimetic macrocycle, wherein the peptidomimetic macrocycle is

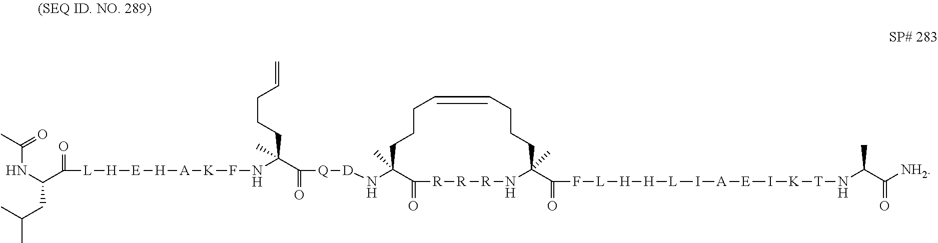

194. A peptidomimetic macrocycle, wherein the peptidomimetic macrocycle is

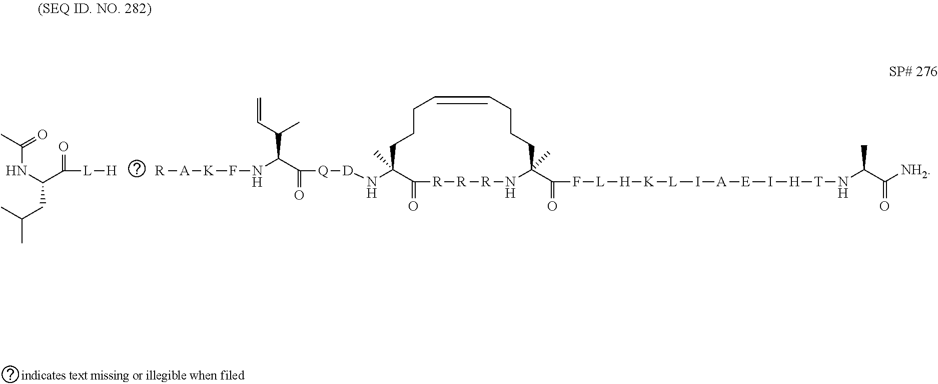

195. A peptidomimetic macrocycle, wherein the peptidomimetic macrocycle is

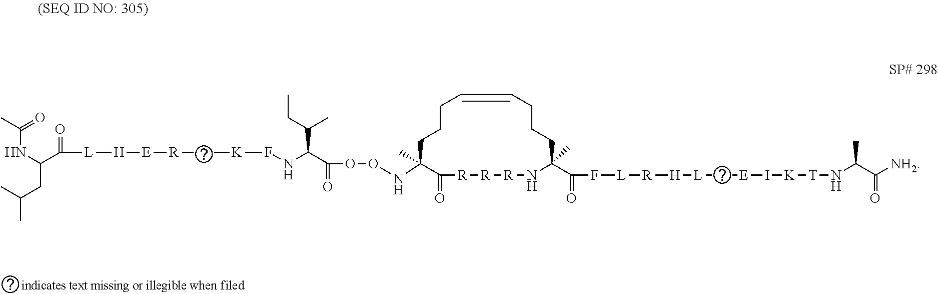

196. A peptidomimetic macrocycle, wherein the peptidomimetic macrocycle is

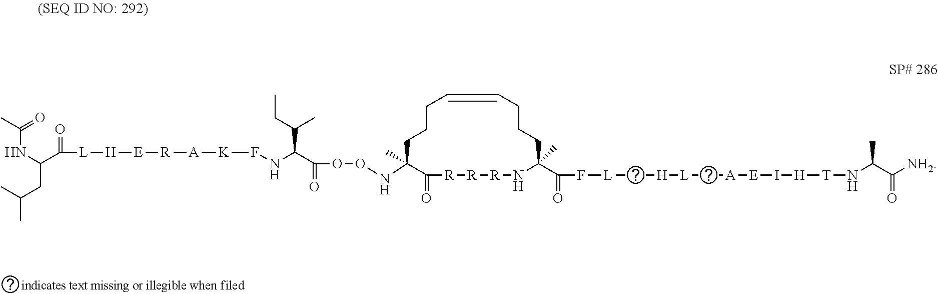

197. A peptidomimetic macrocycle, wherein the peptidomimetic macrocycle is

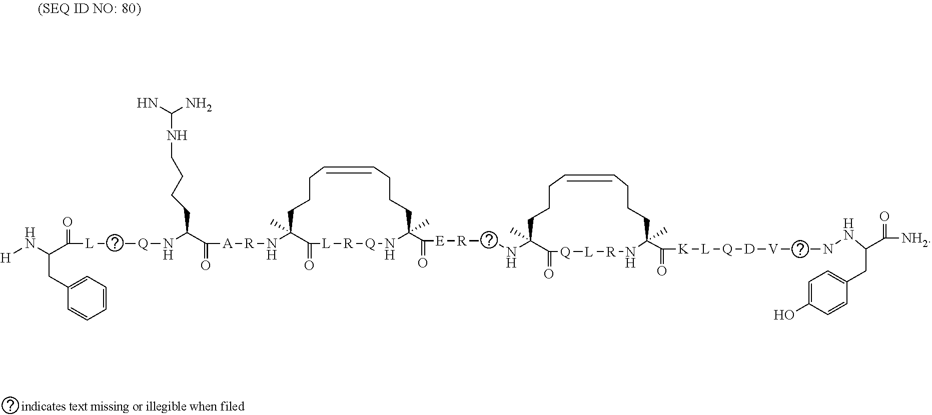

198. A peptidomimetic macrocycle, wherein the peptidomimetic macrocycle is

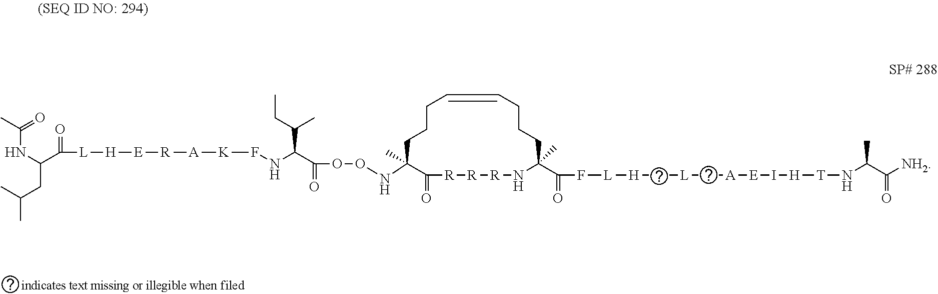

199. A peptidomimetic macrocycle, wherein the peptidomimetic macrocycle is

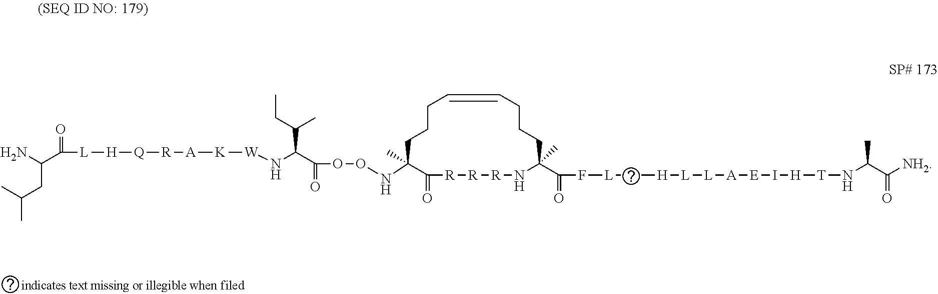

200. A peptidomimetic macrocycle, wherein the peptidomimetic macrocycle is

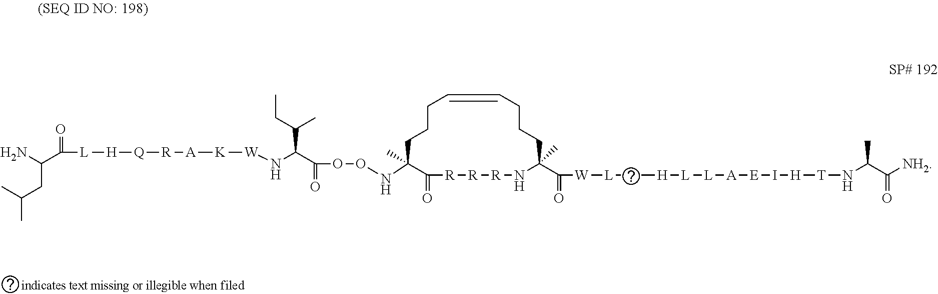

201. A peptidomimetic macrocycle, wherein the peptidomimetic macrocycle is

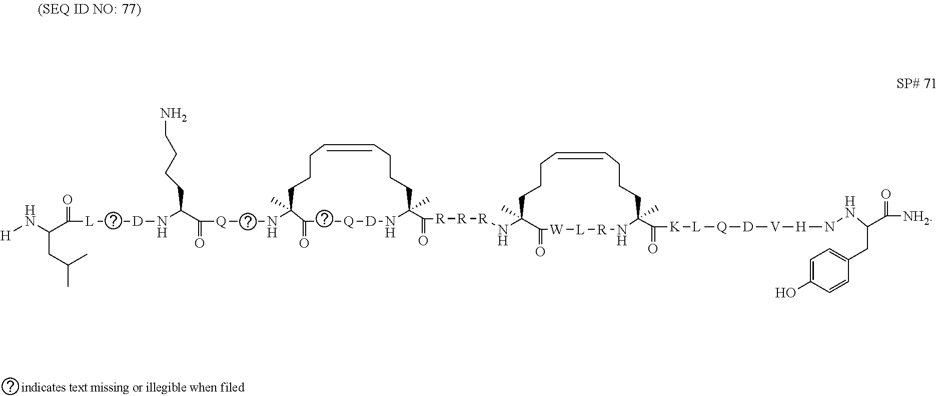

202. A peptidomimetic macrocycle, wherein the peptidomimetic macrocycle is

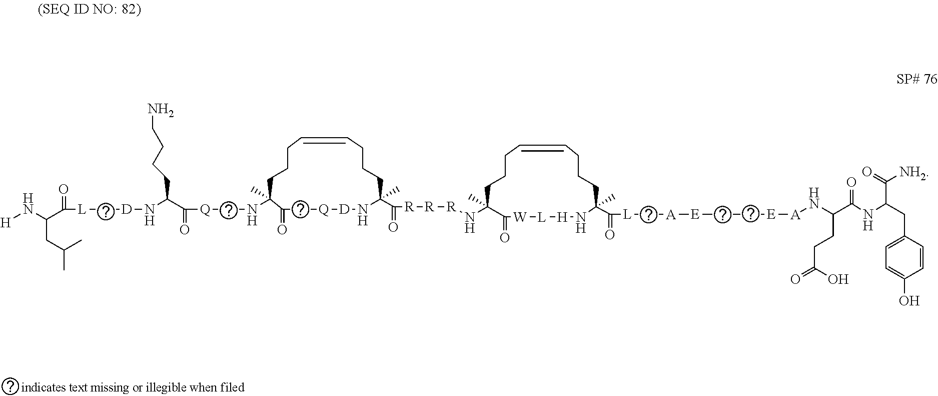

203. A peptidomimetic macrocycle, wherein the peptidomimetic macrocycle is

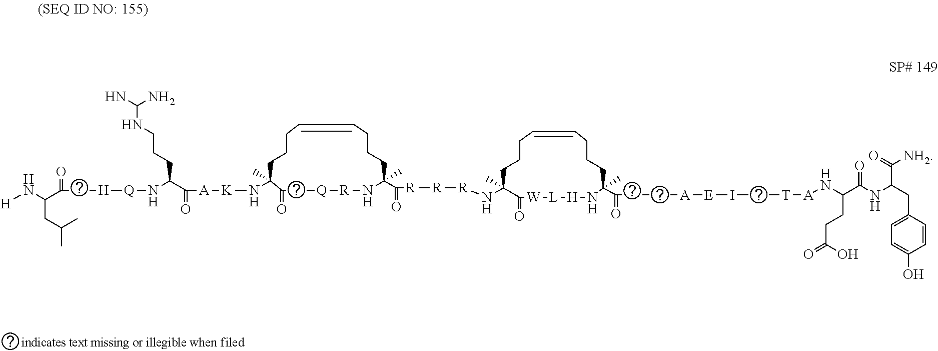

204. A peptidomimetic macrocycle, wherein the peptidomimetic macrocycle is

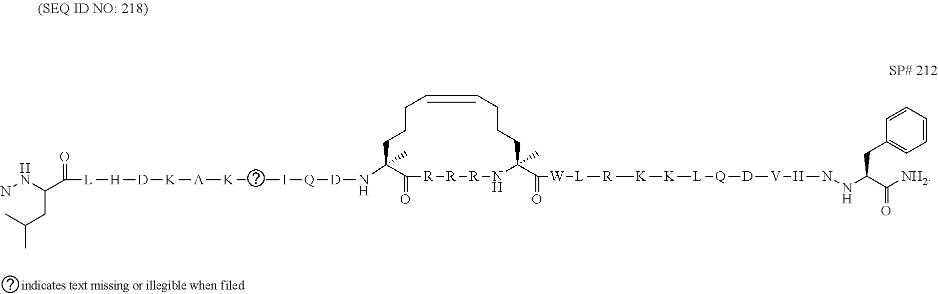

205. A peptidomimetic macrocycle, wherein the peptidomimetic macrocycle is

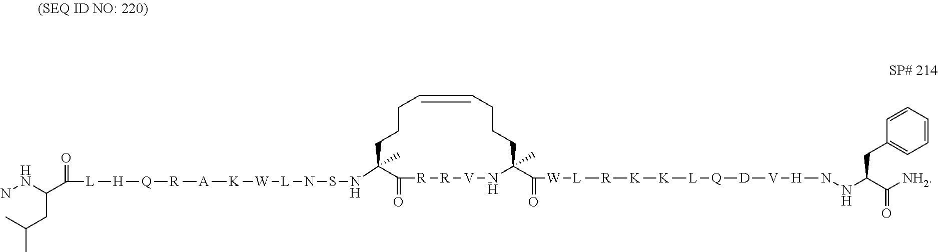

206. A peptidomimetic macrocycle, wherein the peptidomimetic macrocycle is

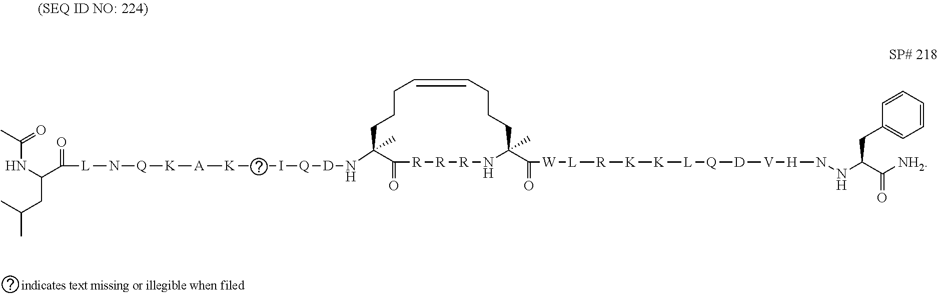

207. A peptidomimetic macrocycle, wherein the peptidomimetic macrocycle is

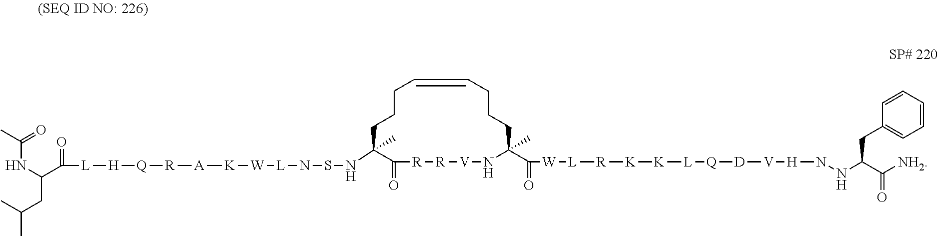

208. A peptidomimetic macrocycle, wherein the peptidomimetic macrocycle is

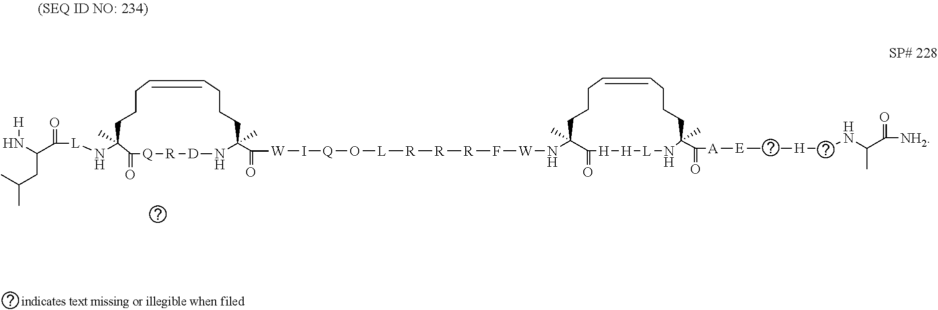

209. A peptidomimetic macrocycle, wherein the peptidomimetic macrocycle is

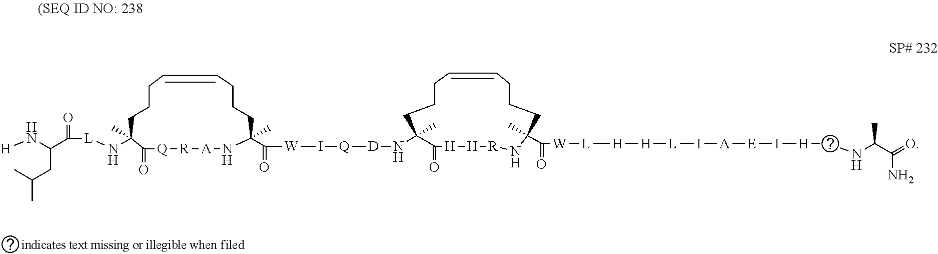

210. A peptidomimetic macrocycle, wherein the peptidomimetic macrocycle is

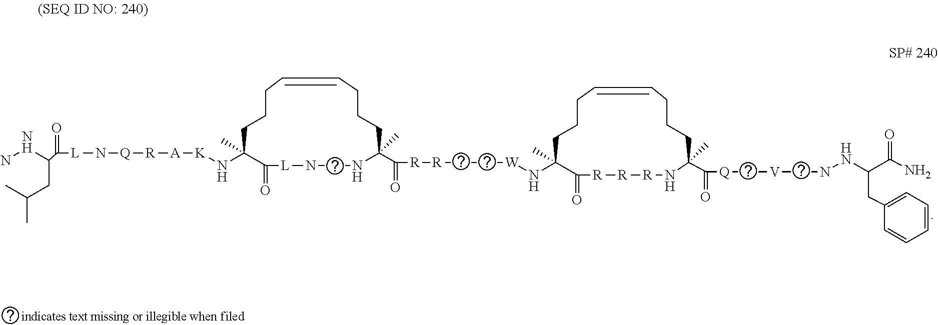

211. A peptidomimetic macrocycle, wherein the peptidomimetic macrocycle is

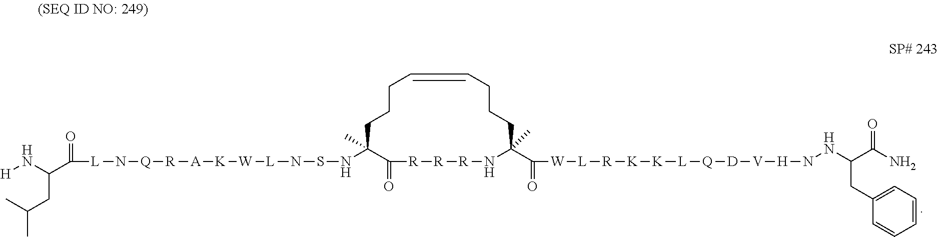

212. A peptidomimetic macrocycle, wherein the peptidomimetic macrocycle is

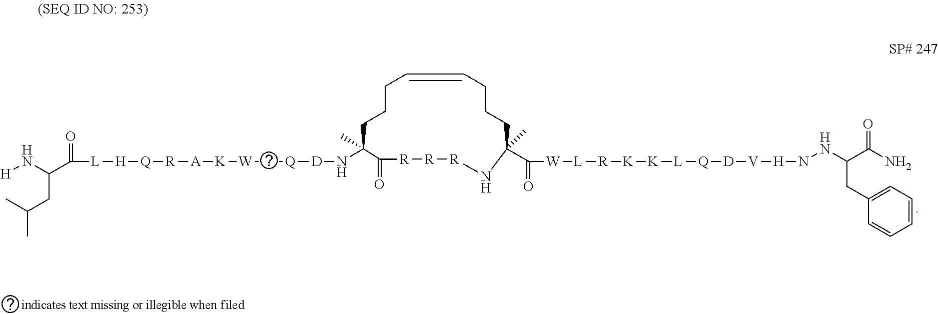

213. A peptidomimetic macrocycle, wherein the peptidomimetic macrocycle is

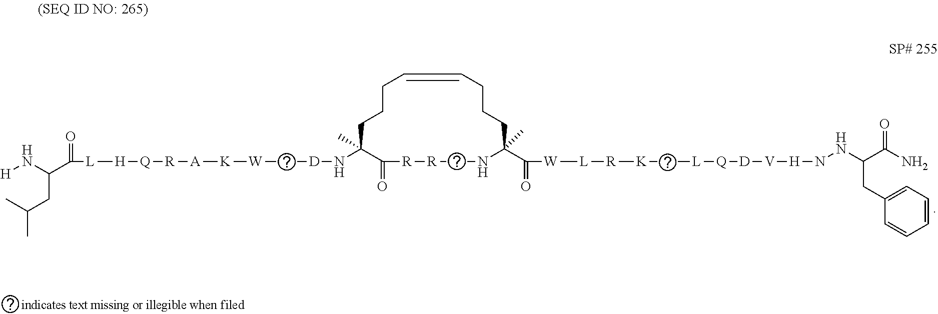

214. A peptidomimetic macrocycle, wherein the peptidomimetic macrocycle is

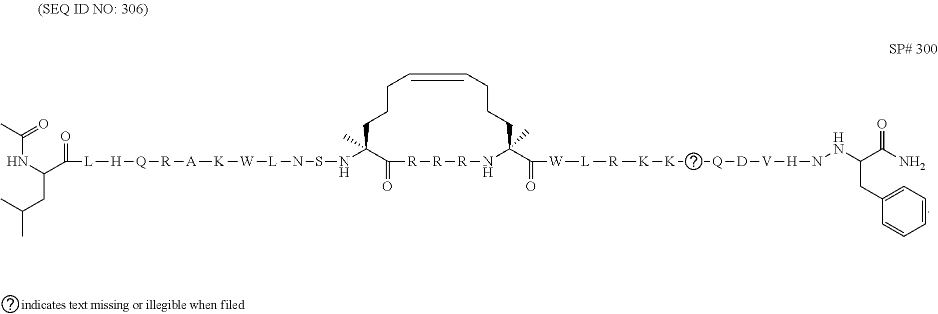

215. A peptidomimetic macrocycle, wherein the peptidomimetic macrocycle is

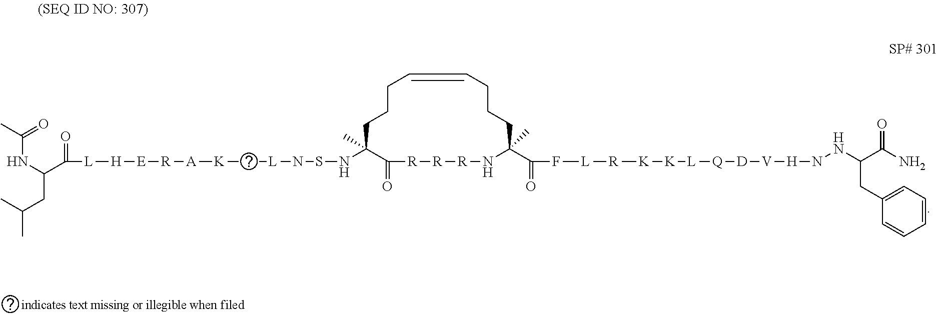

216. A peptidomimetic macrocycle, wherein the peptidomimetic macrocycle is

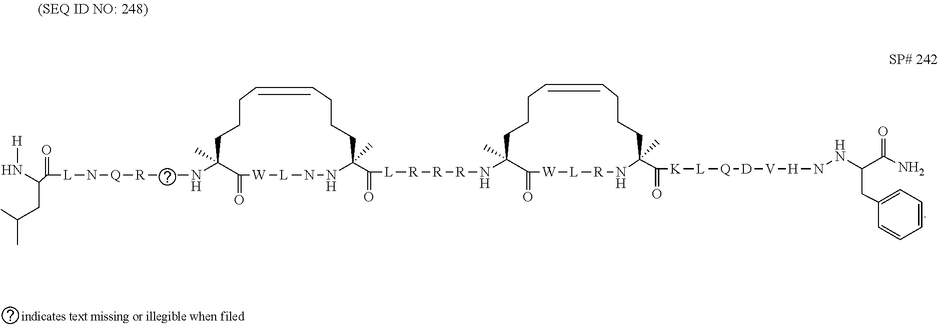

217. A peptidomimetic macrocycle, wherein the peptidomimetic macrocycle is

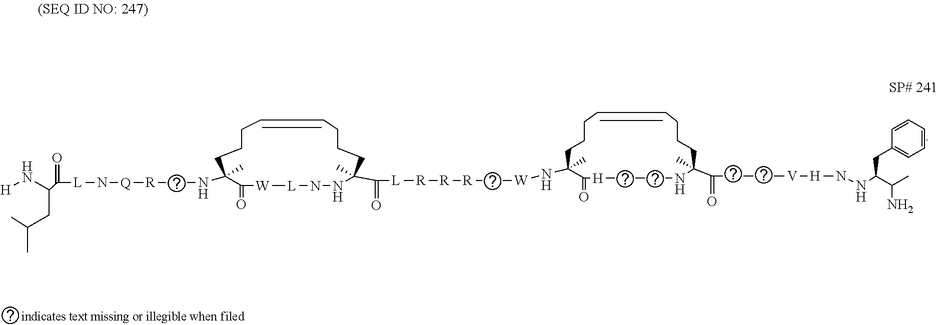

218. A peptidomimetic macrocycle, wherein the peptidomimetic macrocycle is

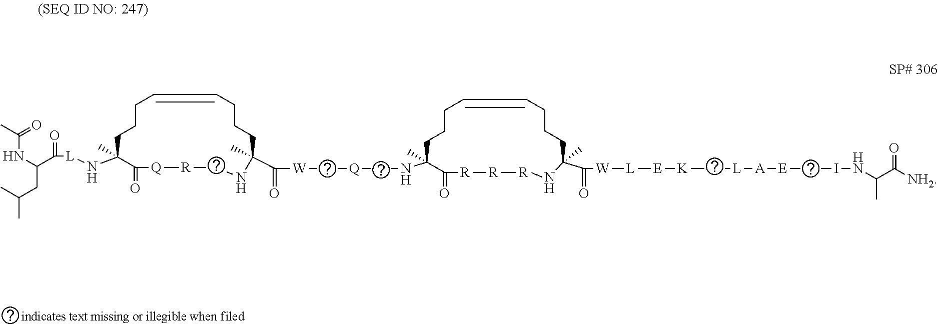

219. A peptidomimetic macrocycle, wherein the peptidomimetic macrocycle is

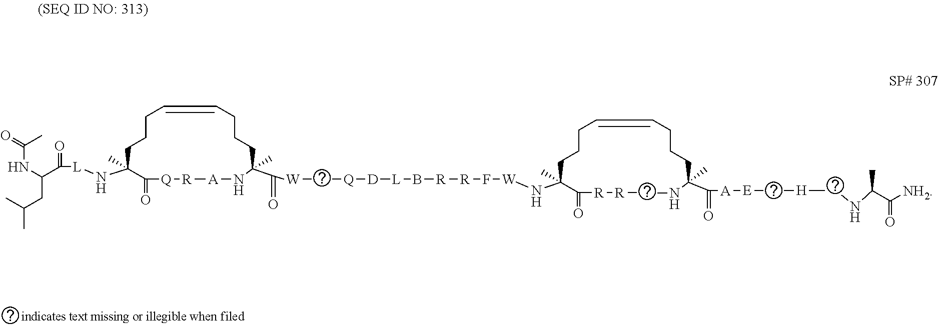

220. A peptidomimetic macrocycle, wherein the peptidomimetic macrocycle is

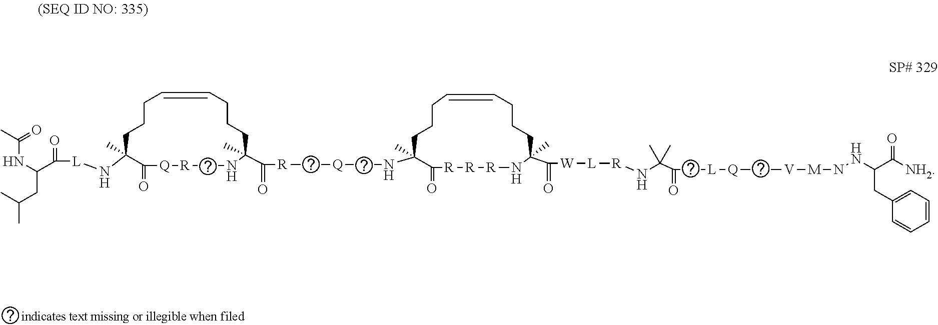

221. A peptidomimetic macrocycle, wherein the peptidomimetic macrocycle is

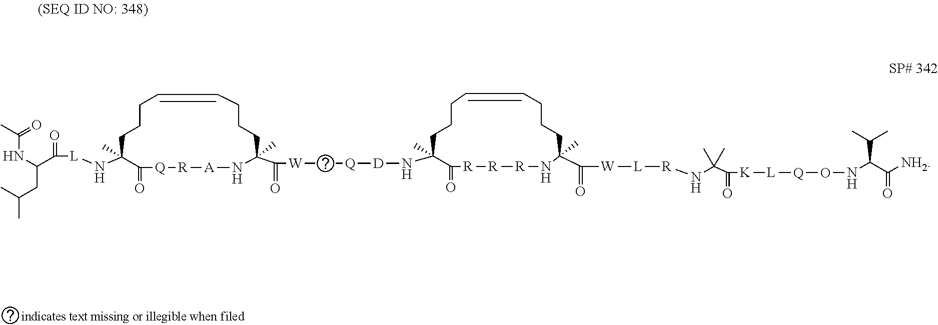

222. A peptidomimetic macrocycle, wherein the peptidomimetic macrocycle is

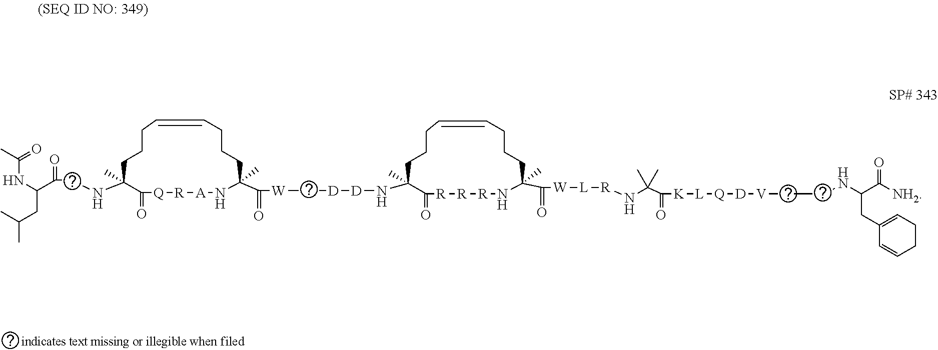

223. A peptidomimetic macrocycle, wherein the peptidomimetic macrocycle is

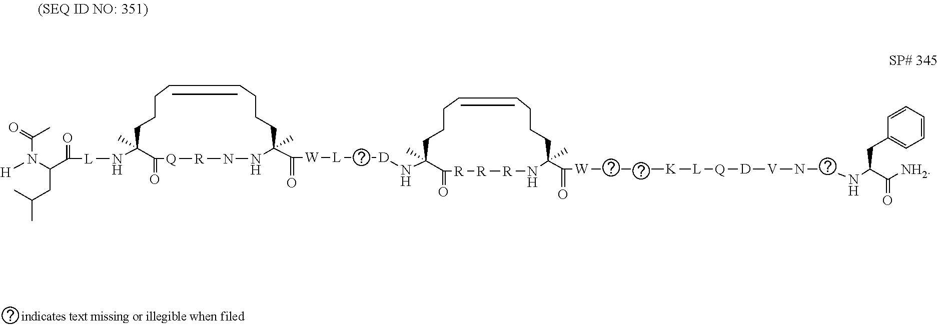

224. A peptidomimetic macrocycle, wherein the peptidomimetic macrocycle s

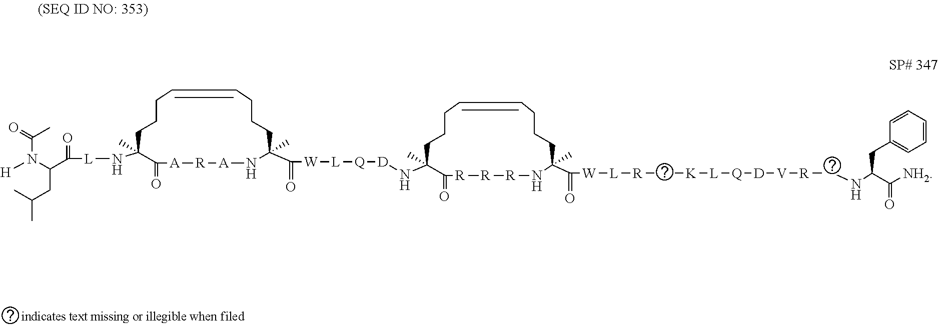

225. A peptidomimetic macrocycle, wherein the peptidomimetic macrocycle is

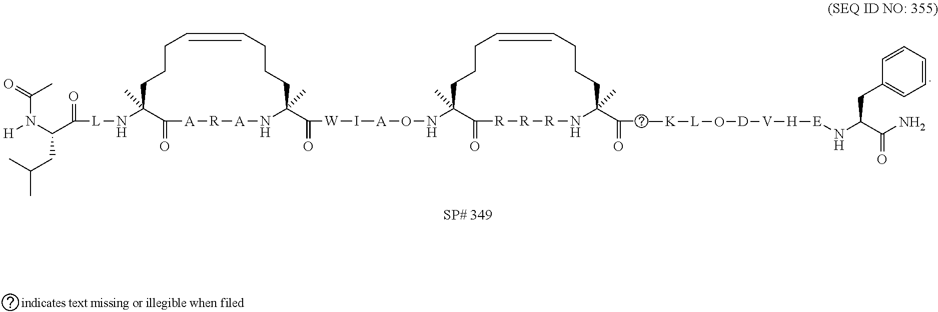

226. A peptidomimetic macrocycle, wherein the peptidomimetic macrocycle is

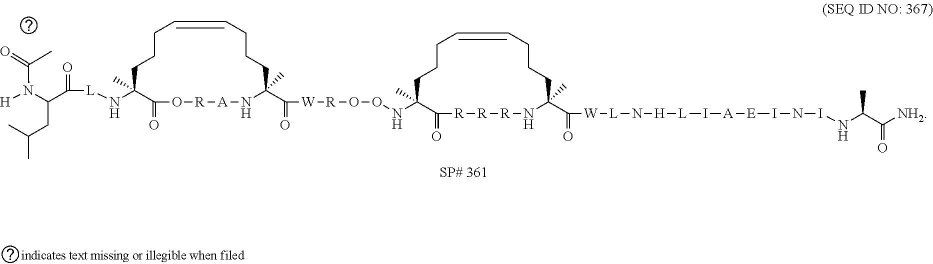

227. A peptidomimetic macrocycle, wherein the peptidomimetic macrocycle is

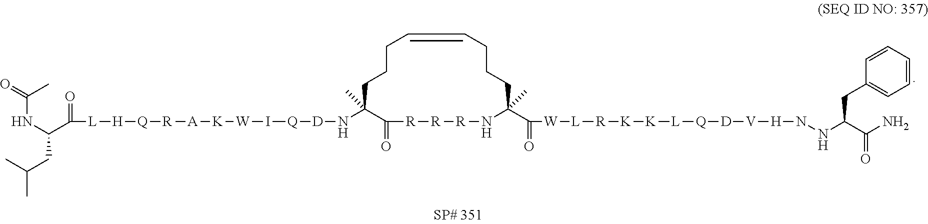

228. A peptidomimetic macrocycle, wherein the peptidomimetic macrocycle is

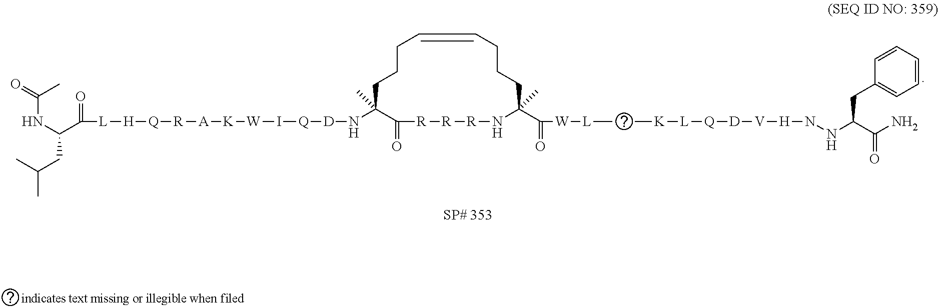

229. A peptidomimetic macrocycle, wherein the peptidomimetic macrocycle is

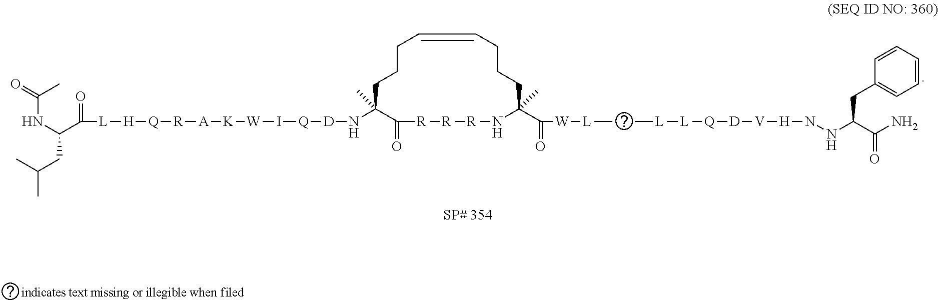

230. A peptidomimetic macrocycle, wherein the peptidomimetic macrocycle is

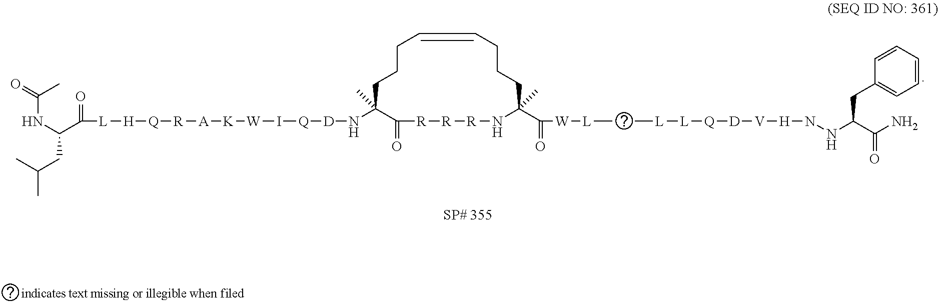

231. A peptidomimetic macrocycle, wherein the peptidomimetic macrocycle is

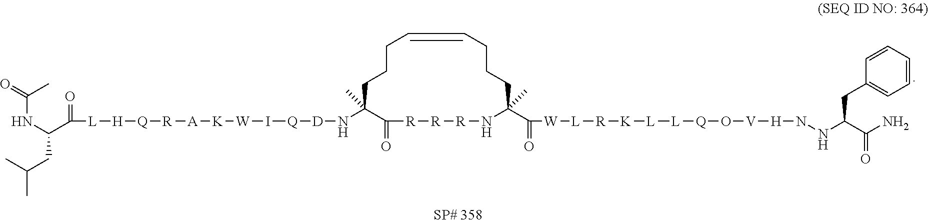

232. A peptidomimetic macrocycle, wherein the peptidomimetic macrocycle is

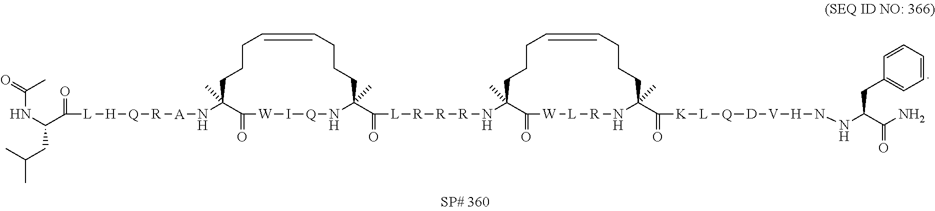

233. A peptidomimetic macrocycle, wherein the peptidomimetic macrocycle is

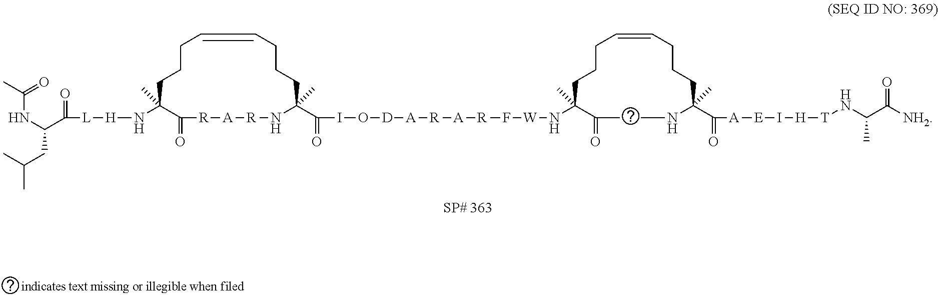

234. A peptidomimetic macrocycle, wherein the peptidomimetic macrocycle is

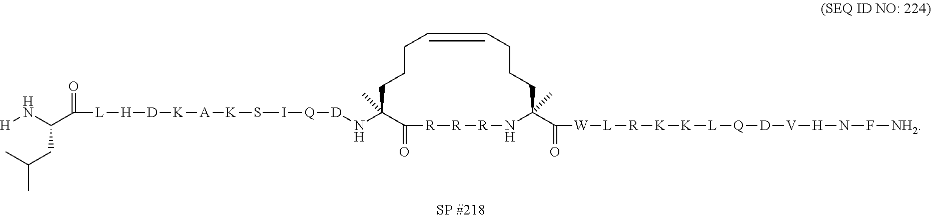

235. A peptidomimetic macrocycle, wherein the peptidomimetic macrocycle is

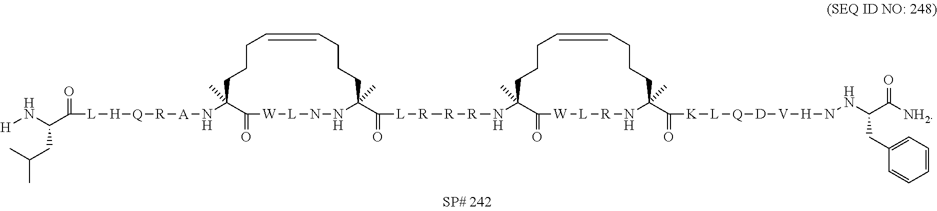

236. A peptidomimetic macrocycle, wherein the peptidomimetic macrocycle is

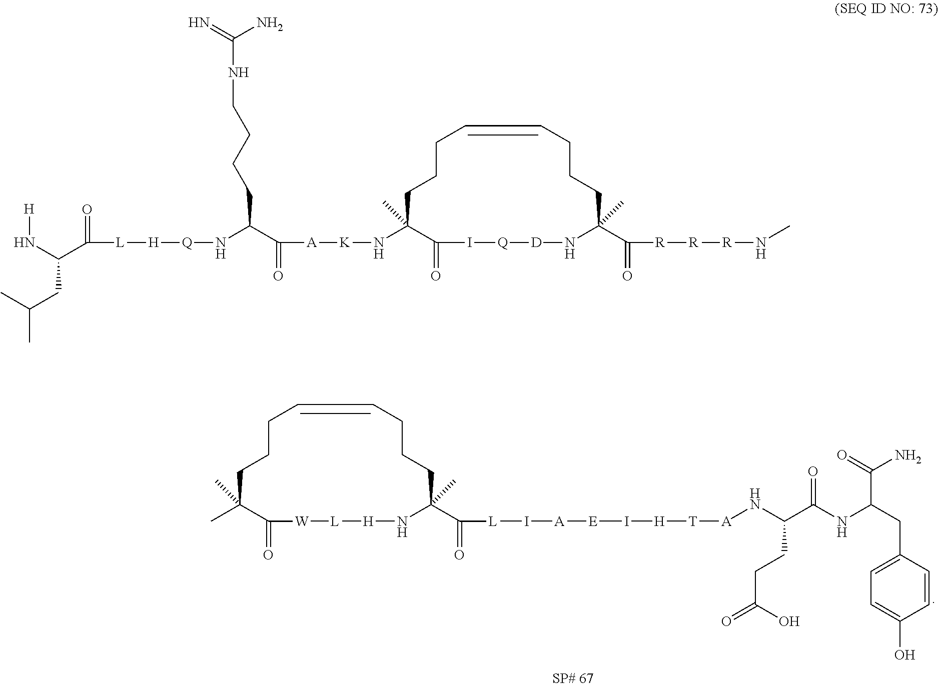

237. A peptidomimetic macrocycle, wherein the peptidomimetic macrocycle is

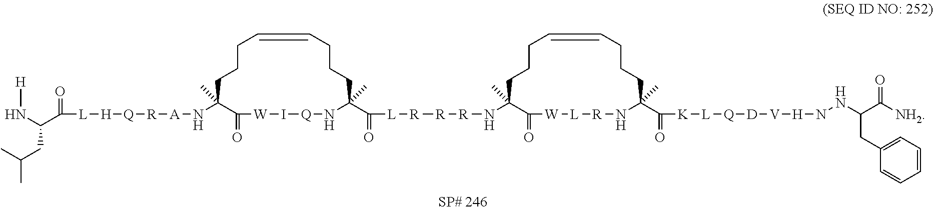

238. A pharmaceutical composition comprising a peptidomimetic macrocycle of any one of claims 1-237, and a pharmaceutically acceptable excipient.

239. A peptidomimetic macrocycle or pharmaceutical composition of any one of claims 1-238, for use in the treatment of a disease.

240. A peptidomimetic macrocycle or pharmaceutical composition of any one of claims 1-238, for use in the manufacture of a medicament for treatment of a disease.

241. Use of a peptidomimetic macrocycle or pharmaceutical composition of any one of claims 1-238, for the manufacture of a medicament for treatment of a disease.

242. Use of a peptidomimetic macrocycle or pharmaceutical composition of any one of claims 1-238, for the treatment of a subject with a disease.

243. A method of preparing a composition comprising a peptidomimetic macrocycle of Formula (IV): 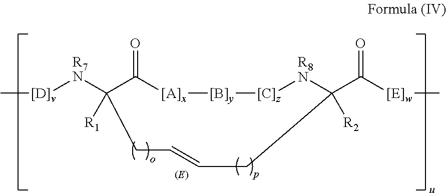
comprising an amino acid sequence that has about 60%, 65%, 70%, 75%, 80%, 85%, 90%, 95%, 96%, 97%, 98%, 99% or 100% sequence identity to a sequence of Table 1a, 1b, 2a, or 2b, wherein the peptidomimetic macrocycle comprises at least two non-natural amino acids connected by a macrocycle-forming linker, the method comprising treating a compound of Formula (V) 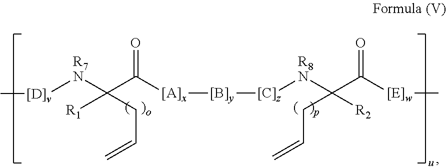
with a catalyst to result in the compound of Formula (IV)
wherein in the compound(s) of Formulae (IV) and (V)
each A, C, D, and E is independently an amino acid;
each B is independently an amino acid, 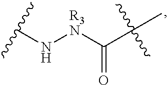
[—NH-L₃-CO—], [—NH-L₃-SO₂—], or [—NH-L₃-];
each R₁ and R₂ are independently —H, alkyl, alkenyl, alkynyl, arylalkyl, cycloalkyl, cycloalkylalkyl, heteroalkyl, or heterocycloalkyl, unsubstituted or substituted with halogen;
or at least one of R₁ and R₂ forms a macrocycle-forming linker L′ connected to the alpha position of one of the D or E amino acids;
each R₃ is independently —H, alkyl, alkenyl, alkynyl, arylalkyl, heteroalkyl, cycloalkyl, heterocycloalkyl, cycloalkylalkyl, cycloaryl, or heterocycloaryl, optionally substituted with R₅; each L′ is independently a macrocycle-forming linker of the formula -L₁-L₂-;
each L₁, L₂ and L₃ are independently alkylene, alkenylene, alkynylene, heteroalkylene, cycloalkylene, heterocycloalkylene, cycloarylene, heterocycloarylene, or [—R₄—K—R₄′-]ₙ,
each being optionally substituted with R₅;
each R₄ and R_{4′} is independently alkylene, alkenylene, alkynylene, heteroalkylene, cycloalkylene, heterocycloalkylene, arylene, or heteroarylene;
each K is independently O, S, SO, SO₂, CO, CO₂ or CONR₃;
each R₅ is independently halogen, alkyl, —OR₆, —N(R₆)₂, —SR₆, —SOR₆, —SO₂R₆, —CO₂R₆, a fluorescent moiety, a radioisotope or a therapeutic agent;
each R₆ is independently —H, alkyl, alkenyl, alkynyl, arylalkyl, cycloalkylalkyl, heterocycloalkyl, a fluorescent moiety, a radioisotope or a therapeutic agent;
each R₇ is independently —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with a D residue;
each R₈ is independently —H, alkyl, alkenyl, alkynyl, arylalkyl, cycloalkyl, heteroalkyl, cycloalkylalkyl, heterocycloalkyl, cycloaryl, or heterocycloaryl, optionally substituted with R₅, or part of a cyclic structure with an E residue;
each v and w is independently an integer from 1-1000;
u is an integer from 1-10;
each x, y and z is independently an integer from 0-10;
each n is independently an integer from 1-5;
each o is independently an integer from 1-15;
each p is independently an integer from 1-15;
“(E)” indicates a trans double bond; and
one or more of the amino acids A, C and/or B when B is an amino acid, present in the compounds of Formulae (IV) and (V), has a side chain bearing a protecting group.

244. The method of claim 243, wherein the protecting group is a nitrogen atom protecting group.

245. The method of claim 243 or 244, wherein the protecting group is a Boc group.

246. The method of any one of claims 243-245, wherein the side chain of the amino acid bearing the protecting group comprises a protected indole.

247. The method of claim 246, wherein the amino acid bearing the protecting group on its side chain is tryptophan (W) that is protected by the protecting group on its indole nitrogen.

248. The method of claim 247, wherein the amino acid bearing the protecting group on its side chain is tryptophan (W) that is protected on its indole nitrogen by a Boc group.

249. The method of any one of claims 243-248, wherein after the step of contacting the compound of Formula (V) with catalyst the compound of Formula (IV) is obtained in equal or higher amounts than a corresponding compound which is a Z isomer.

250. The method of claim 249, wherein after the step of contacting the compound of Formula (V) with catalyst the compound of Formula (IV) is obtained in a 2, 3, 4, 5, 6, 7, 8, 9, or 10-fold higher amount than the corresponding compound which is a Z isomer.

251. The method of any one of claims 243-250, wherein the catalyst is a ruthenium catalyst.

252. The method of any one of claims 243-251, further comprising the step of treating the compounds of Formula (IV) with a reducing agent or an oxidizing agent.

253. The method of any one of claims 243-252, wherein the compound of Formula (V) is attached to a solid support.

254. The method of any one of claims 243-252, wherein the compound of Formula (V) is not attached to a solid support.

255. The method of any one of claims 243-254, further comprising removing the protecting group(s) from the compounds of Formula (IV).

256. The method of any one of claims 243-255, wherein the ring closing metathesis is conducted at a temperature ranging from about 20° C. to about 80° C.

257. A method for treating a condition characterized by increased or decreased activity or production of PTH or PTHrP in a subject in need thereof, comprising administering to the subject an effective amount of a peptidomimetic macrocycle or pharmaceutical composition of any one of claims 1-238.

258. A method for treating a condition characterized by increased or decreased activity or production of PTH or PTHrP in a subject in need thereof, comprising administering to the subject an effective amount of a composition comprising a peptidomimetic macrocycle of any one of claims 6, 11, 46, 75-80, 168-171, and 187-237.

259. The method of claim 257 or 258, wherein the condition is hypoparathyroidism.

260. The method of claim 257 or 258, wherein the condition is hyperparathyroidism or hypercalcemia.

261. The method of claim 260, wherein the condition is primary hyperparathyroidism.

262. The method of claim 261, wherein the subject suffers from a parathyroid adenoma, parathyroid hyperplasia, or a parathyroid carcinoma.

263. The method of claim 262, wherein the parathyroid carcinoma is inoperable parathyroid tumor.

264. The method of claim 263, wherein the inoperable parathyroid tumor is metaphyseal chondrodysplasia.

265. The method of claim 261, wherein the subject suffers from a multiple endocrine neoplasia or familial hyperparathyroidism.

266. The method of claim 260, wherein the condition is secondary hyperparathyroidism.

267. The method of claim 266, wherein the subject suffers from a renal disorder or vitamin D deficiency.

268. The method of claim 267, wherein the renal disorder is chronic kidney disease.

269. The method of claim 268, wherein the chronic kidney disease is in stage 1, 2, 3 or 4.

270. The method of any one of claims 267-269, wherein the subject is undergoing dialysis.

271. The method of claim 260, wherein the condition is tertiary hyperparathyroidism.

272. A method for decreasing the activity of PTH or PTHrP in a subject in need thereof, comprising administering to the subject an effective amount of a peptidomimetic macrocycle or pharmaceutical composition of any one of claims 2-238.

273. A method for decreasing the activity of PTH or PTHrP in a subject in need thereof, comprising administering to the subject an effective amount of a peptidomimetic macrocycle or pharmaceutical composition of any one of claims 6, 11, 46, 75-80, 168-171, and 187-237.

274. A method for treating a condition characterized by a decrease in adipose tissue or insufficient adipose tissue or a decrease in skeletal muscle tissue or insufficient skeletal muscle tissue comprising administering to the subject an effective amount of a peptidomimetic macrocycle or pharmaceutical composition of any one of claims 2-238.

275. A method for treating a condition characterized by a decrease in adipose tissue or insufficient adipose tissue or a decrease in skeletal muscle tissue or insufficient skeletal muscle tissue comprising administering to the subject an effective amount of a peptidomimetic macrocycle or pharmaceutical composition of any one of claims 6, 11, 46, 75-80, 168-171, and 187-237.

276. The method of any one of claims 272-275, wherein the condition is cachexia.

277. The method of any one of claims 272-275, wherein the condition is cancer cachexia.

278. The method of any one of claims 272-275, wherein the condition is an increased resting energy expenditure level.

279. The method of any one of claims 272-275, wherein the condition is an increased thermogenesis by brown fat.

280. A method for treating a condition of skin or hair, comprising administering to the subject an effective amount of a peptidomimetic macrocycle or pharmaceutical composition of any one of claims 2-238.

281. A method for treating a condition of skin or hair, comprising administering to the subject an effective amount of a composition comprising a peptidomimetic macrocycle of any one of claims 6, 11, 46, 75-80, 168-171, and 187-237.

282. The method of claim 280 or 281, wherein the condition is insufficient hair growth.

283. The method of claim 280 or 281, wherein the condition is psoriasis.

284. A method for treating a condition characterized by a decrease in bone mass or insufficient bone mass in a subject, comprising administering to the subject an effective amount of a composition comprising a peptidomimetic macrocycle of any one of claims 2-238.

285. A method for treating a condition characterized by a decrease in bone mass or insufficient bone mass in a subject, comprising administering to the subject an effective amount of a composition comprising a peptidomimetic macrocycle of any one of claims 6, 11, 46, 75-80, 168-171, and 187-237.

286. The method of claim 284 or 285, wherein the condition is osteoporosis.

287. The method of claim 284 or 285, wherein the condition is osteopenia.

288. The method of any one of claims 257-287, wherein the peptidomimetic macrocycle is administered parenterally.

289. The method of any one of claims 257-287, wherein the peptidomimetic macrocycle is administered subcutaneously.

290. The method of any one of claims 257-287, wherein the peptidomimetic macrocycle is administered intravenously.

291. The method of any one of claims 288-290, wherein the administering is no more frequently than once daily, no more frequently than every other day, no more frequently than three times weekly, no more frequently than twice weekly, no more frequently than weekly, or no more frequently than every other week.

292. The method of any one of claims 288-290, wherein the administering is no more frequently than three times weekly.

293. The method of any one of claims 288-290, wherein the administering is no more frequently than weekly, for example once weekly.
